(19) United States
(12) Patent Application Publication
(10) Pub. No.: US 2022/0169654 A1
(43) Pub. Date: Jun. 2, 2022

(54) New Organic Compounds

(71) Applicant: Servier Pharmaceuticals LLC, Boston, MA (US)

(72) Inventors: Zenon D. KONTEATIS, Chatham, NJ (US); Mingzong LI, Medford, MA (US); Samuel K. REZNIK, Cambridge, MA (US); Zhihua SUI, Cambridge, MA (US)

(73) Assignee: Servier Pharmaceuticals LLC, Boston, MA (US)

(21) Appl. No.: 17/534,510

(22) Filed: Nov. 24, 2021

Related U.S. Application Data
(60) Provisional application No. 63/118,252, filed on Nov. 25, 2020.

(51) Int. Cl. C07D 487/04; A61P 35/00

(52) U.S. Cl. CPC C07D 487/04; A61P 35/00

(57) ABSTRACT

The present application provides MAT2A inhibitor compounds that are useful as therapeutic agents. In some embodiments, the compounds are of Formula I, or pharmaceutically acceptable salts thereof:

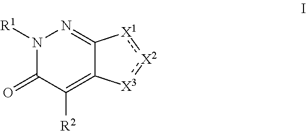

wherein R¹, R², and X¹-X³ are defined herein.

### CROSS-REFERENCE TO RELATED APPLICATIONS

This application claims priority to U.S. Provisional Patent Application No. 63/118,252, filed Nov. 25, 2020, the disclosure of which is incorporated by reference herein.

### TECHNICAL FIELD

The present application relates to organic compounds useful for therapy and/or prophylaxis in a mammal, and in particular to inhibitors of MAT2A enzyme which are useful for treating certain cancers.

### BACKGROUND

Methionine adenosyltransferase (MAT), also known as S-adenosylmethionine synthetase, is a cellular enzyme that catalyzes the synthesis of S-adenosyl methionine (SAM) from methionine and ATP. MAT is considered the rate-limiting step of the methionine cycle. SAM is the propylamino donor in polyamine biosynthesis and the principal methyl donor for DNA methylation and is involved in gene transcription and cellular proliferation as well as the production of secondary metabolites. Two genes, MAT1A and MAT2A, encode two distinct catalytic MAT isoforms. Because MAT isoforms differ in catalytic kinetics and regulatory properties, MAT1A-expressing cells have considerably higher SAM levels than do MAT2A-expressing cells.

It has been reported that cancer cell lines that are MTAP deficient are particularly sensitive to inhibition of MAT2A. MTAP (methylthioadenosine phosphorylase) is an enzyme widely expressed in normal tissues that catalyzes the conversion of methylthioadenosine (MTA) into adenine and 5-methylthioribose-1-phosphate. Many human and murine malignant cells lack MTAP activity. MTAP deficiency is not only found in tissue culture cells but the deficiency is also present in primary leukemias, gliomas, melanomas, pancreatic cancers, non-small cell lung cancers, bladder cancers, astrocytomas, osteosarcomas, head and neck cancers, myxoid chondrosarcomas, ovarian cancers, endometrial cancers, breast cancers, soft tissue sarcomas, non-Hodgkin lymphomas, and mesotheliomas.

New compounds for treating cancers are needed.

### SUMMARY

In some embodiments, the application provides compounds of Formula I, or pharmaceutically acceptable salts thereof, wherein R¹, R², X¹, X², and X³ are defined herein.

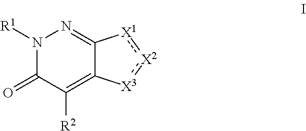

In other embodiments, the application provides compounds of Formula II, or pharmaceutically acceptable salts thereof, wherein R¹, R², X¹, and X² are defined herein.

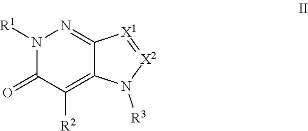

In further embodiments, the application provides compounds of Formula III, or pharmaceutically acceptable salts thereof, wherein R¹-R⁵ are defined herein.

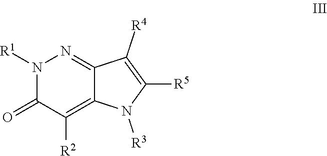

In still other embodiments, the application provides compounds of Formula IV, or pharmaceutically acceptable salts thereof, wherein R¹-R³ and X² are defined herein.

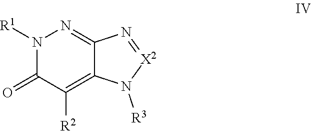

In yet further embodiments, the application provides compounds of Formula V, or pharmaceutically acceptable salts thereof, wherein R¹-R³ and R⁵ are defined herein.

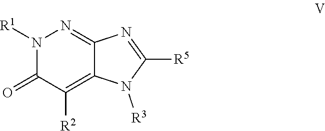

In other embodiments, the application provides compounds of Formula VI, or pharmaceutically acceptable salts thereof, wherein R¹-R⁴ are defined herein.

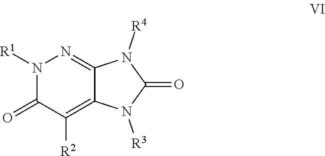

In further embodiments, the application provides compounds of Formula VII, or pharmaceutically acceptable salts thereof, wherein R¹-R³ are defined herein.

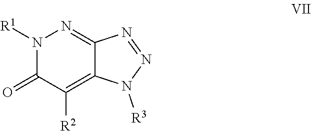

In still other embodiments, the application provides compounds of Formula VIII, or pharmaceutically acceptable salts thereof, wherein R¹-R⁴ are defined herein.

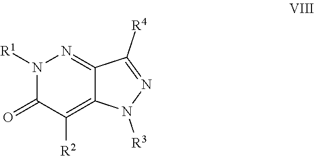

In yet further embodiments, the application provides compounds of Formula IX, or pharmaceutically acceptable salts thereof, wherein R¹, R², R³, R⁴, R^{4′}, R⁵, and R^{5′} are defined herein.

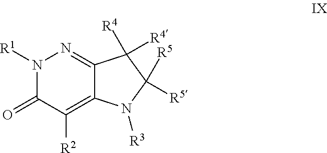

In other embodiments, the application provides compounds of Formula X, or pharmaceutically acceptable salts thereof, wherein R¹-R⁵ are defined herein.

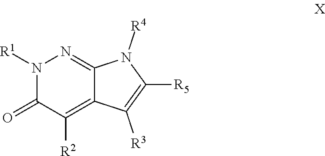

In further embodiments, the application provides compounds of Formula XIa, XIb, or XIc, or pharmaceutically acceptable salts thereof, wherein R², R³, R^{A}, X¹, X², and R⁷ are defined herein.

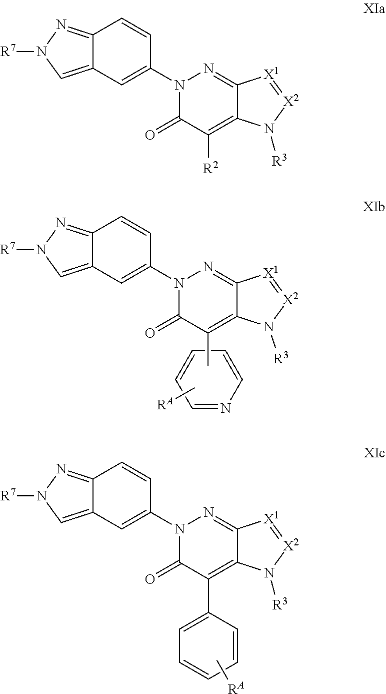

In yet other embodiments, the application provides compounds of Formula XIIa, XIIb, or XIIc, or pharmaceutically acceptable salts thereof, wherein R², R³, R⁷, R^{A}, X¹, and X² are defined herein.

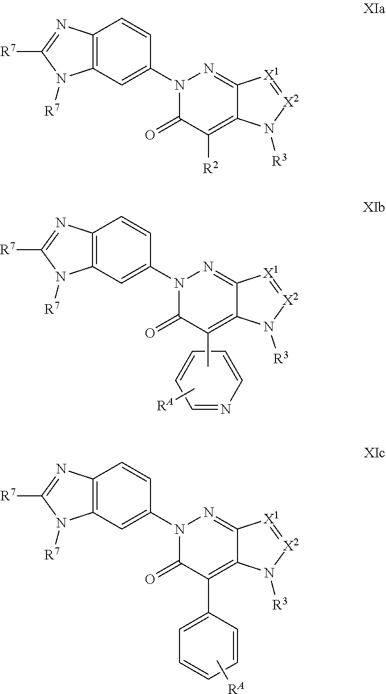

In still further embodiments, the application provides compounds of Formula XIIIa, XIIIb, or XIIIc, or pharmaceutically acceptable salts thereof, wherein R², R^{A}, X¹, and X² are defined herein.

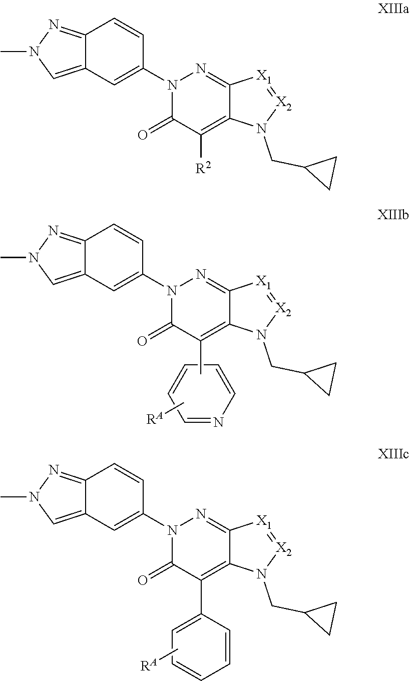

In other embodiments, the application provides methods for inhibiting the synthesis of S-adenosyl methionine (SAM) in a subject in need thereof, comprising administering to the subject a SAM-synthesis inhibiting amount of a compound described herein, or a pharmaceutically acceptable salt thereof.

In further embodiments, the application provides methods for treating a disease or condition that responds to the inhibition of MAT2A in a subject in need thereof, comprising administering to the subject an effective amount of a compound disclosed herein, or a pharmaceutically acceptable salt thereof. In some aspects, the disease or condition is cancer. In some aspects, the cancer is an MTAP deleted cancer. In other aspects the cancer is MTAP wild type (WT).

In other embodiments, the application provides methods for treating a cancer in a subject suffering therefrom, wherein the cancer is characterized by a reduction or absence of methylthioadenosine phosphorylase (MTAP) gene expression, the absence of the MTAP gene, or reduced function of MTAP protein, the method comprising administering to the subject a therapeutically effective amount of a compound disclosed herein, or a pharmaceutically acceptable salt thereof.

In yet other embodiments, the application provides pharmaceutical compositions comprising a compound disclosed herein, or a pharmaceutically acceptable salt thereof, and a pharmaceutically acceptable carrier.

In still further embodiments, the application provides a compound disclosed herein, or a pharmaceutically acceptable salt thereof, for inhibiting the synthesis of S-adenosyl methionine (SAM) in a subject. In some aspects, a reduction in the concentration of SAM results in a therapeutic benefit in a patient. In some aspects, the application provides compounds disclosed herein, or pharmaceutically acceptable salts thereof, for treating a disease or condition in a subject suffering therefrom, wherein the disease or condition is responsive to a reduction in the concentration of SAM. In some embodiments, the compounds disclosed herein, or pharmaceutically acceptable salts thereof, reduce the plasma concentration of SAM in a subject.

In other embodiments, the application provides compounds disclosed herein, or pharmaceutically acceptable salts thereof, for treating a disease or condition in a subject suffering therefrom, wherein the disease or condition is responsive to the inhibition of MAT2A.

In further embodiments, the application provides compounds disclosed herein, or pharmaceutically acceptable salts thereof, for treating a cancer in a subject suffering therefrom, wherein the cancer is characterized by a reduction or absence of methylthioadenosine phosphorylase (MTAP) expression, the absence of the MTAP gene, or reduced function of MTAP protein.

### DETAILED DESCRIPTION OF ILLUSTRATIVE EMBODIMENTS

### Definitions

The singular forms “a”, “an” and “the” include the plural reference, and reference to a particular numerical value includes at least that value, unless the context clearly indicates otherwise.

When a value is expressed as an approximation by use of “about” it will be understood that the particular value forms another embodiment. In general, “about” indicates approximations that can vary depending on the desired properties sought to be obtained by the disclosed subject matter and is to be interpreted in the specific context in which it is used, based on its function. The person skilled in the art will be able to interpret this as a routine matter. In some cases, the number of significant figures used for a particular value may be one non-limiting method of determining the extent of “about.” In other cases, gradations used in a series of values may be used to determine the intended range for “about” for each value. Where present, all ranges are inclusive and combinable. That is, references to values stated in ranges include every value within that range.

When a list is presented, unless stated otherwise, it is to be understood that each individual element of that list and combinations of that list is to be interpreted as a separate embodiment. For example, “A, B, or C” is to be interpreted as including the embodiments, “A,” “B,” “C,” “A or B,” “A or C,” “B or C,” or “A, B, or C.”

“Alkyl” comprises a branched or unbranched, saturated aliphatic hydrocarbon group. In some embodiments an alkyl group comprises 1 to 12 carbon atoms unless otherwise specified. In other embodiments, an alkyl group comprises 1 to 6 carbon atoms, such as a C₁₋₆-alkyl. Alkyl may be used as part of another term, for example “alkylamino”. Examples of particular alkyl groups are methyl, ethyl, n-propyl, isopropyl, n-butyl, iso-butyl, sec-butyl, tert-butyl, n-pentyl, 2-methylbutyl, 2,2-dimethylpropyl, n-hexyl, 2-methylpentyl, 2,2-dimethylbutyl, n-heptyl, 3-heptyl, 2-methylhexyl, and the like. Unless specified, substituted alkyl groups may contain one, two, three or four substituents which may be the same or different, and are chosen from alkenyl, alkynyl, amino, aryl, carbocyclyl, heterocyclyl, heteroaryl, D, alkoxy, halo, —S(O)₀₋₂NHC₁₋₆alkyl, —S(O)₀₋₂(C₁₋₆alkyl), —S(O)₀₋₂(C₁₋₆alkyl)_{2′} —S(O)₀₋₂(C₆₋₁₄aryl), —S(O)₀₋₂(heteroaryl), —S(O)₀₋₂(heterocyclyl), —C(O)(C₁₋₆alkyl), —C(O)(C₃₋₁₄carbocyclyl), —C(O)(C₆₋₁₄aryl), —C(O)(heteroaryl), —C(O)(heterocyclyl), —C(O)NHC₁₋₆alkyl, —C(O)NH(C₁₋₆alkyl)₂ or other groups specified.

Similarly, C₀₋₆ alkyl may be used when a moiety is directly bound to another group and refers to the absence of an alkyl group, i.e., C₀, and/or the presence of a C₁₋₆-alkyl group as defined above. For example, a C₀alkyl-NH₂ group is a —NH₂ group.

“Alkenyl” comprises a branched or unbranched, unsaturated (fully or partially) hydrocarbon group. In some embodiments, the alkenyl group comprises 2 to 12 carbon atoms unless otherwise specified. In other embodiments, an alkenyl group comprises 2 to 6 carbon atoms, such as a C₂₋₆-alkenyl. The alkenyl group comprises at least one double bond. In some embodiments, the alkenyl group comprises one double bond. In other embodiments, the alkenyl group comprises two double bonds. In further embodiments, the alkenyl group comprises three double bonds. Examples of particular alkenyl groups are ethenyl, propenyl, butenyl, pentenyl, hexenyl, heptenyl, and the like. In some aspects, the alkenyl group is an ethenyl, propenyl, butenyl, pentenyl, or hexenyl. Unless specified, substituted alkenyl groups may contain one, two, three or four substituents which may be the same or different, and are chosen from alkyl, alkynyl, amino, aryl, carbocyclyl, heterocyclyl, heteroaryl, D, alkoxy, halo, —S(O)₀₋₂NHC₁₋₆alkyl, —S(O)₀₋₂(C₁₋₆alkyl), —S(O)₀₋₂(C₁₋₆alkyl)_{2′} —S(O)₀₋₂(C₆₋₁₄aryl), —S(O)₀₋₂(heteroaryl), —S(O)₀₋₂(heterocyclyl), —C(O)(C₁₋₆alkyl), —C(O)(C₃₋₁₄carbocyclyl), —C(O)(C₆₋₁₄aryl), —C(O)(heteroaryl), —C(O)(heterocyclyl), —C(O)NHC₁₋₆alkyl, —C(O)NH(C₁₋₆alkyl)₂ or other groups specified.

“Alkynyl” comprises a branched or unbranched, unsaturated (fully or partially) hydrocarbon group. In some embodiments, the alkynyl group comprises 2 to 12 carbon atoms unless otherwise specified. In other embodiments, an alkynyl group comprises 2 to 6 carbon atoms, such as a C₂₋₆-alkynyl. The alkenyl group comprises at least one triple bond. In some embodiments, the alkenyl group comprises one triple bond. In other embodiments, the alkenyl group comprises two triple bonds. In further embodiments, the alkynyl group comprises three double bonds. Examples of particular alkynyl groups are ethynyl, propynyl, butynyl, pentynyl, hexynyl, heptynyl, and the like. In some aspects, the alkenyl group is an ethynyl, propynyl, butynyl, pentynyl, or hexynyl. Unless specified, substituted alkynyl groups may contain one, two, three or four substituents which may be the same or different, and are chosen from alkyl, alkenyl, amino, aryl, carbocyclyl, heterocyclyl, heteroaryl, D, alkoxy, halo, —S(O)₀₋₂NHC₁₋₆alkyl, —S(O)₀₋₂(C₁₋₆alkyl), —S(O)₀₋₂(C₁₋₆alkyl)_{2′} —S(O)₀₋₂(C₆₋₁₄aryl), —S(O)₀₋₂(heteroaryl), —S(O)₀₋₂(heterocyclyl), —C(O)(C₁₋₆alkyl), —C(O)(C₃₋₁₄carbocyclyl), —C(O)(C₆₋₁₄aryl), —C(O)(heteroaryl), —C(O)(heterocyclyl), —C(O)NHC₁₋₆alkyl, —C(O)NH(C₁₋₆alkyl)₂ or other groups specified.

“Amino” means primary (i.e. —NH₂), secondary (i.e. —NRH) and tertiary (i.e. —NRR) amines in which R is H, alkyl, a carbocycle, a heterocycle, carbocycle-substituted alkyl or heterocycle-substituted alkyl. Particular secondary and tertiary amines are alkylamine, dialkylamine, arylamine, diarylamine, aralkylamine and diaralkylamine wherein the alkyl is as herein defined and optionally substituted. Particular secondary and tertiary amines are methylamine, ethylamine, propylamine, isopropylamine, phenylamine, benzylamine dimethylamine, diethylamine, dipropylamine and diisopropylamine.

“Aryl” when used alone or as part of another term means a carbocyclic aromatic group. The aryl group may optionally be fused to another aryl group to form a multicyclic aryl moiety. In some embodiments, an aryl comprises 5 to 14 carbon atoms, such as a C₅₋₁₄-aryl. In some embodiments, the aryl is a C₅aryl, C₆aryl, C₇aryl, C₈aryl, C₉aryl, C₁₀aryl, C₁₁aryl, C₁₂aryl, C₁₃aryl, or C₁₄aryl. In other embodiments, the aryl is C₅₋₈aryl, such as C₅₋₆aryl, C₅₋₇aryl, C₆₋₇aryl, C₆₋₈aryl, or C₇₋₈aryl. Particular aryl groups are phenyl, naphthyl, biphenyl, phenanthrenyl, naphthacenyl, and the like (see e.g. Lang's Handbook of Chemistry (Dean, J. A., ed) 13^{th} ed. Table 7-2 [1985]). In some aspects, the aryl is phenyl. The aryl may be substituted with one, two, three, four or five, for example 1-2, 1-3 or 1-4 substituents chosen, unless otherwise specified, from alkyl, alkenyl, alkynyl, amino, aryl, carbocyclyl, heterocyclyl, heteroaryl, —C₀₋₆alkyl-C₆₋₁₄aryl, —C₁₋₆alkylOH, D, deuterated C₁₋₆alkyl, C₂₋₆haloalkenyl, alkoxy, deuterated C₁₋₆alkoxy, halo, C₁₋₆haloalkyl, C₁₋₆haloalkoxy, —C₀₋₆alkyl-C₃₋₁₂carbocyclyl, —C₀₋₆alkyl-C₆₋₁₄aryl, —C₀₋₆alkyl-O—C₆₋₁₄aryl, —C₀₋₆alkyl-(3- to 14-membered heteroaryl), —C₀₋₆alkyl-(3- to 14-membered heterocyclyl), —C₀₋₆alkyl-O-(3- to 14-membered heterocyclyl), —S(O)₀₋₂NHC₁₋₆alkyl, —S(O)₀₋₂(C₁₋₆alkyl), —S(O)₀₋₂(C₁₋₆alkyl)_{2′} —S(O)₀₋₂(C₆₋₁₄aryl), —S(O)₀₋₂(heteroaryl), —S(O)₀₋₂(heterocyclyl), —C(O)(C₁₋₆alkyl), —C(O)(C₃₋₁₄carbocyclyl), —C(O)(C₆₋₁₄aryl), —C(O)(heteroaryl), —C(O)(heterocyclyl), —C(O)NHC₁₋₆alkyl, —C(O)NH(C₁₋₆alkyl)₂, —C₀₋₆alkyl-NH₂, —C₀₋₆alkyl-NH(alkyl), or —C₀₋₆alkyl-N(alkyl)₂ or other groups specified.

“Carbocyclyl” alone and when used as a moiety in a complex group refers to a mono-, bi-, or tricyclic carbon ring that is saturated or partially unsaturated. In some embodiments, a carbocyclyl has 3 to 14 carbon atoms, such as a C₃₋₁₄carbocyclyl. In some embodiments, the carbocyclyl is C₃carbocyclyl, C₄carbocyclyl, C₅carbocyclyl, C₆carbocyclyl, C₇carbocyclyl, C₈carbocyclyl, C₉carbocyclyl, C₁₀carbocyclyl, C₁₁carbocyclyl, C₁₂carbocyclyl, C₁₃carbocyclyl, or C₁₄carbocyclyl. In other embodiments, the carbocyclyl is C₃₋₈carbocyclyl, C₃₋₇carbocyclyl, C₃₋₆carbocyclyl, C₃₋₅carbocyclyl, C₃₋₄carbocyclyl, C₄₋₈carbocyclyl, C₄₋₇carbocyclyl, C₄₋₆carbocyclyl, C₄₋₅carbocyclyl, C₅₋₈carbocyclyl, C₅₋₇carbocyclyl, C₅₋₆carbocyclyl, C₆₋₈carbocyclyl, C₆₋₇carbocyclyl, or C₆₋₈carbocyclyl. Particular saturated carbocyclyl groups are cyclopropyl, cyclobutyl, cyclopentyl or cyclohexyl groups. A particular saturated carbocyclyl is cyclopropyl. Another particular saturated carbocyclyl is cyclohexyl. Particular partially unsaturated carbocyclic groups are cyclobutenyl, cyclopentenyl, cyclohexenyl, or cycloheptenyl. Unless specified, substituted carbocyclyl groups may contain one, two, three or four substituents which may be the same or different, and are chosen from alkyl, alkenyl, alkynyl, amino, aryl, carbocyclyl, heterocyclyl, heteroaryl, —C₀₋₆alkyl-C₆₋₁₄aryl, —C₁₋₆alkylOH, D, deuterated C₁₋₆alkyl, C₂₋₆haloalkenyl, alkoxy, deuterated C₁₋₆alkoxy, halo, C₁₋₆haloalkyl, C₁₋₆haloalkoxy, —C₀₋₆alkyl-C₃₋₁₂carbocyclyl, —C₀₋₆alkyl-C₆₋₁₄aryl, —C₀₋₆alkyl-O—C₆₋₁₄aryl, —C₀₋₆alkyl-(3- to 14-membered heteroaryl), —C₀₋₆alkyl-(3- to 14-membered heterocyclyl), —C₀₋₆alkyl-O-(3- to 14-membered heterocyclyl), —S(O)₀₋₂NHC₁₋₆alkyl, —S(O)₀₋₂(C₁₋₆alkyl), —S(O)₀₋₂(C₁₋₆alkyl)_{2′} —S(O)₀₋₂(C₆₋₁₄aryl), —S(O)₀₋₂(heteroaryl), —S(O)₀₋₂(heterocyclyl), —C(O)(C₁₋₆alkyl), —C(O)(C₃₋₁₄carbocyclyl), —C(O)(C₆₋₁₄aryl), —C(O)(heteroaryl), —C(O)(heterocyclyl), —C(O)NHC₁₋₆alkyl, —C(O)NH(C₁₋₆alkyl)₂, —C₀₋₆alkyl-NH₂, —C₀₋₆alkyl-NH(alkyl), or —C₀₋₆alkyl-N(alkyl)₂ or other groups specified.

“Heterocyclyl” alone and when used as a moiety in a complex group refer to any mono-, bi-, or tricyclic group that is saturated. In some embodiments, a heterocyclyl comprises 3-14 ring atoms. In some embodiments, a heterocyclyl comprises 3 ring atoms, 4 ring atoms, 5 ring atoms, 6 ring atoms, 7 ring atoms, 8 ring atoms, 9 ring atoms, 10 ring atoms, 11 ring atoms, 12 ring atoms, 13 ring atoms, or 14 ring atoms. A heterocyclyl comprises ring atoms that are carbon and at least one heteroatom. The ring atom may be nitrogen, sulfur or oxygen. In some embodiments, the heterocyclyl comprises one nitrogen, one sulfur, one oxygen, two nitrogen atoms, three nitrogen atoms, four nitrogen atoms, two oxygen atoms, three oxygen atoms, two sulfur atoms, three sulfur atoms, or combinations thereof. In other embodiments, the heterocyclyl comprises 1-4 heteroatoms. In further embodiments, the heterocyclyl comprises 1-3 heteroatoms, 1-2 heteroatoms, 2-4 heteroatoms, 2-3 heteroatoms, or 3-4 heteroatoms. In other embodiments, the heterocyclyl comprises 1-4 heteroatoms that are, independently, N, O, or S. When the heterocyclyl comprises nitrogen or sulfur atoms, these heteroatoms may optionally be oxidized (e.g. SO, SO₂, or N—O), and any nitrogen heteroatom may optionally be quaternized. Particular non-aromatic heterocycles are morpholinyl, pyrrolidinyl, pyrrolidinyl, oxiranyl, oxetanyl, tetrahydrofuranyl, tetrahydropyranyl, thiiranyl, thietanyl, tetrahydrothiophenyl, aziridinyl, azetidinyl, piperazinyl and piperidinyl. Unless specified, substituted heterocyclyl groups may contain one, two, three or four substituents which may be the same or different, and are chosen from alkyl, alkenyl, alkynyl, amino, aryl, carbocyclyl, heterocyclyl, heteroaryl, —C₀₋₆alkyl-C₆₋₁₄aryl, —C₁₋₆alkylOH, D, deuterated C₁₋₆alkyl, C₂₋₆haloalkenyl, alkoxy, deuterated C₁₋₆alkoxy, halo, C₁₋₆haloalkyl, C₁₋₆haloalkoxy, —C₀₋₆alkyl-C₃₋₁₂carbocyclyl, —C₀₋₆alkyl-C₆₋₁₄aryl, —C₀₋₆alkyl-O—C₆₋₁₄aryl, —C₀₋₆alkyl-(3- to 14-membered heteroaryl), —C₀₋₆alkyl-(3- to 14-membered heterocyclyl), —C₀₋₆alkyl-O-(3- to 14-membered heterocyclyl), —S(O)₀₋₂NHC₁₋₆alkyl, —S(O)₀₋₂(C₁₋₆alkyl), —S(O)₀₋₂(C₁₋₆alkyl)_{2′} —S(O)₀₋₂(C₆₋₁₄aryl), —S(O)₀₋₂(heteroaryl), —S(O)₀₋₂(heterocyclyl), —C(O)(C₁₋₆alkyl), —C(O)(C₃₋₁₄carbocyclyl), —C(O)(C₆₋₁₄aryl), —C(O)(heteroaryl), —C(O)(heterocyclyl), —C(O)NHC₁₋₆alkyl, —C(O)NH(C₁₋₆alkyl)₂, —C₀₋₆alkyl-NH₂, —C₀₋₆alkyl-NH(alkyl), or —C₀₋₆alkyl-N(alkyl)₂ or other groups specified.

“Heteroaryl” alone and when used as a moiety in a complex group refers to any mono-, bi-, or tricyclic aromatic ring system that is unsaturated or partially unsaturated. In some embodiments, a heterocyclyl comprises 3-14 ring atoms. In some embodiments, a heteroaryl comprises 3 ring atoms, 4 ring atoms, 5 ring atoms, 6 ring atoms, 7 ring atoms, 8 ring atoms, 9 ring atoms, 10 ring atoms, 11 ring atoms, 12 ring atoms, 13 ring atoms, or 14 ring atoms. A heteroaryl comprises ring atoms that are carbon and at least one heteroatom. The ring atom may be nitrogen, sulfur or oxygen. In some embodiments, the heteroaryl comprises one nitrogen, one sulfur, one oxygen, two nitrogen atoms, three nitrogen atoms, four nitrogen atoms, two oxygen atoms, three oxygen atoms, two sulfur atoms, three sulfur atoms, or combinations thereof. In other embodiments, the heteroaryl comprises 1-4 heteroatoms. In further embodiments, the heteroaryl comprises 1-3 heteroatoms, 1-2 heteroatoms, 2-4 heteroatoms, 2-3 heteroatoms, or 3-4 heteroatoms. In other embodiments, the heteroaryl comprises 1-4 heteroatoms that are, independently, N, O, or S. When the heteroaryl comprises nitrogen or sulfur atoms, these heteroatoms may optionally be oxidized (e.g. SO, SO₂, or N—O), and any nitrogen heteroatom may optionally be quaternized. Included in the definition are any bicyclic groups where any of the above heteroaryl rings are fused to a benzene ring. In some embodiments, the heteroaryl is 2H-pyranyl, thienyl, furyl, 2,3-dihydrofuranyl, imidazolyl, pyrazolyl, thiazolyl, isothiazolyl, oxazolyl, isoxazolyl, triazolyl, thiadiazolyl, oxadiazolyl, tetrazolyl, thiatriazolyl, oxatriazolyl, pyridyl, pyrimidyl, pyrazinyl, pyridazinyl, thiazinyl, oxazinyl, triazinyl, thiadiazinyl, oxadiazinyl, dithiazinyl, dioxazinyl, oxathiazinyl, tetrazinyl, thiatriazinyl, oxatriazinyl, dithiadiazinyl, imidazolinyl, dihydropyrimidyl, tetrahydropyrimidyl, tetrazolo[1,5-b]pyridazinyl and purinyl, as well as benzo-fused derivatives, for example benzoxazolyl, benzofuryl, benzothiazolyl, benzothiadiazolyl, benzotriazolyl, benzoimidazolyl and indolyl. In other embodiments, the heteroaryl is 1,3-thiazol-2-yl, 4-(carboxymethyl)-5-methyl-1,3-thiazol-2-yl, 4-(carboxymethyl)-5-methyl-1,3-thiazol-2-yl sodium salt, 1,2,4-thiadiazol-5-yl, 3-methyl-1,2,4-thiadiazol-5-yl, 1,3,4-triazol-5-yl, 2-methyl-1,3,4-triazol-5-yl, 2-hydroxy-1,3,4-triazol-5-yl, 2-carboxy-4-methyl-1,3,4-triazol-5-yl sodium salt, 2-carboxy-4-methyl-1,3,4-triazol-5-yl, 1,3-oxazol-2-yl, 1,3,4-oxadiazol-5-yl, 2-methyl-1,3,4-oxadiazol-5-yl, 2-(hydroxymethyl)-1,3,4-oxadiazol-5-yl, 1,2,4-oxadiazol-5-yl, 1,3,4-thiadiazol-5-yl, 2-thiol-1,3,4-thiadiazol-5-yl, 2-(methylthio)-1,3,4-thiadiazol-5-yl, 2-amino-1,3,4-thiadiazol-5-yl, 1H-tetrazol-5-yl, 1-methyl-1H-tetrazol-5-yl, 1-(1-(dimethylamino)ethyl)-1H-tetrazol-5-yl, 1-(carboxymethyl)-1H-tetrazol-5-yl, 1-(methylsulfonic acid)-1H-tetrazol-5-yl, 2-methyl-1H-tetrazol-5-yl, 1,2,3-triazol-5-yl, 1-methyl-1,2,3-triazol-5-yl, 2-methyl-1,2,3-triazol-5-yl, 4-methyl-1,2,3-triazol-5-yl, pyrid-2-yl N-oxide, 6-methoxy-2-(N-oxide)-pyridaz-3-yl, 6-hydroxypyridaz-3-yl, 1-methylpyrid-2-yl, 1-methylpyrid-4-yl, 2-hydroxypyrimid-4-yl, 1,4,5,6-tetrahydro-5,6-dioxo-4-methyl-as-triazin-3-yl, 1,4,5,6-tetrahydro-4-(formylmethyl)-5,6-dioxo-as-triazin-3-yl, 2,5-dihydro-5-oxo-6-hydroxy-astriazin-3-yl, 2,5-dihydro-5-oxo-6-hydroxy-2-methyl-as-triazin-3-yl, 2,5-dihydro-5-oxo-6-methoxy-2-methyl-as-triazin-3-yl, 2,5-dihydro-5-oxo-as-triazin-3-yl, 2,5-dihydro-5-oxo-2-methyl-as-triazin-3-yl, 2,5-dihydro-5-oxo-2,6-dimethyl-as-triazin-3-yl, tetrazolo[1,5-b]pyridazin-6-yl, or 8-aminotetrazolo[1,5-b]-pyridazin-6-yl. In further embodiments, the heteroaryl is 4-(carboxymethyl)-5-methyl-1,3-thiazol-2-yl, 1,3,4-triazol-5-yl, 2-methyl-1,3,4-triazol-5-yl, 1H-tetrazol-5-yl, 1-methyl-1H-tetrazol-5-yl, 1-(1-(dimethylamino)eth-2-yl)-1H-tetrazol-5-yl, 1-(carboxymethyl)-1H-tetrazol-5-yl, 1-(methylsulfonic acid)-1H-tetrazol-5-yl, 1,2,3-triazol-5-yl, 1,4,5,6-tetrahydro-5,6-dioxo-4-methyl-as-triazin-3-yl, 1,4,5,6-tetrahydro-4-(2-formylmethyl)-5,6-dioxo-as-triazin-3-yl, 2,5-dihydro-5-oxo-6-hydroxy-2-methyl-as-triazin-3-yl, tetrazolo[1,5-b]pyridazin-6-yl, and 8-aminotetrazolo[1,5-b]pyridazin-6-yl. Unless specified, substituted heteroaryl groups may contain one, two, three or four substituents which may be the same or different, and are chosen from alkyl, alkenyl, alkynyl, amino, aryl, carbocyclyl, heterocyclyl, heteroaryl, —C₀₋₆alkyl-C₆₋₁₄aryl, —C₁₋₆alkylOH, D, deuterated C₁₋₆alkyl, C₂₋₆haloalkenyl, alkoxy, deuterated C₁₋₆alkoxy, halo, C₁₋₆haloalkyl, C₁₋₆haloalkoxy, —C₀₋₆alkyl-C₃₋₁₂carbocyclyl, —C₀₋₆alkyl-C₆₋₁₄aryl, —C₀₋₆alkyl-O—C₆₋₁₄aryl, —C₀₋₆alkyl-(3- to 14-membered heteroaryl), —C₀₋₆alkyl-(3- to 14-membered heterocyclyl), —C₀₋₆alkyl-O-(3- to 14-membered heterocyclyl), —S(O)₀₋₂NHC₁₋₆alkyl, —S(O)₀₋₂(C₁₋₆alkyl), —S(O)₀₋₂(C₁₋₆alkyl)_{2′} —S(O)₀₋₂(C₆₋₁₄aryl), —S(O)₀₋₂(heteroaryl), —S(O)₀₋₂(heterocyclyl), —C(O)(C₁₋₆alkyl), —C(O)(C₃₋₁₄carbocyclyl), —C(O)(C₆₋₁₄aryl), —C(O)(heteroaryl), —C(O)(heterocyclyl), —C(O)NHC₁₋₆alkyl, —C(O)NH(C₁₋₆alkyl)₂, —C₀₋₆alkyl-NH₂, —C₀₋₆alkyl-NH(alkyl), or —C₀₋₆alkyl-N(alkyl)₂ or other groups specified.

The term “—C₀₋₆alkyl-C₆₋₁₄aryl” refers to a group where the point of attachment is through the alkyl group (if present) and the alkyl and aryl groups are defined herein.

The term “—C₁₋₆alkylOH” refers to a group where the point of attachment is through the alkyl group and the alkyl is defined herein. The OH group may be bound to any carbon atom of the alkyl chain.

The abbreviation “D” as used herein refers to deuterium, i.e., an isotope of H or ²H. In some embodiments, each deuterium has an isotopic enrichment factor that is above that of which is naturally occurring. In other embodiments, the isotopic enrichment for each deuterium atom is at least 6000 (90% deuterium incorporation), at least 6333 (95% deuterium incorporation), or at least 6600 (99% deuterium incorporation, or greater.

“Deuterated C₁₋₆alkyl” is an alkyl group as defined herein wherein one or more hydrogen atom is replaced with a deuterium as defined herein. In some embodiments, the deuterated C₁₋₆alkyl comprises one deuterium. In other embodiments, the deuterated C₁₋₆alkyl comprises two deuterium. In further embodiments, the deuterated C₁₋₆alkyl comprises three deuterium. Specific examples of deuterated C₁₋₆alkyl include, without limitation, —CH₂D, —CHD₂, —CD₃, —CH₂CH₂D, —CH₂CHD₂, —CH₂CD₃, —CHDCH₃, OCD₂CH₃, among others.

The term “C₂₋₆haloalkenyl” refers to a group where the point of attachment is through the alkenyl group, the alkenyl is defined herein, and one or more carbon atom is substituted with one or more halo. Examples of C₂₋₆haloalkenyl include C₁₋₆fluoroalkenyl, C₂₋₆chloroalkenyl, C₂₋₆bromoalkenyl, or C₂₋₆iodooalkenyl. In some embodiments, the C₂₋₆haloalkenyl comprises one halo. In other embodiments, the C₂₋₆haloalkenyl comprises two halo. In further embodiments, the C₂₋₆haloalkenyl comprises three halo. Specific examples of C₂₋₆haloalkenyl include, without limitation, —CH═CHF, —CH═CF₂, —CF₃, —CH═CHCH₂F, —CH═CHCHF₂, —CH═CHCF₃, —CH═CFCH₃, —CH₂C═CHF, —CH₂C═CF₂, among others.

“Alkoxy” is an alkyl group comprising an oxygen atom in the backbone of the molecule. In some embodiments an alkoxy group comprises 1 to 12 carbon atoms unless otherwise specified. In other embodiments, an alkoxy group comprises 1 to 6 carbon atoms, such as a C₁₋₆-alkoxy. Examples of particular alkoxy groups are methoxy, ethoxy, n-propoxy, isopropoxy, n-butoxy, iso-butoxy, sec-butoxy, tert-butoxy, n-pentoxy, 2-methylbutoxy, 2,2-dimethylpropoxy, n-hexoxy, 2-methylpentoxy, 2,2-dimethylbutoxy, n-heptoxy, 3-heptoxy, 2-methylhexoxy, and the like. Unless specified, substituted alkyl groups may contain one or two, three or four substituents which may be the same or different, and are chosen, unless otherwise specified, from halogen (F, Cl, Br, I), hydroxy, protected hydroxy, cyano, nitro, alkoxy (for example C₁₋₆ alkoxy), benzyloxy, carboxy, protected carboxy, carboxymethyl, protected carboxymethyl, hydroxymethyl, protected hydroxymethyl, aminomethyl, protected aminomethyl, trifluoromethyl, alkylsulfonylamino, alkylsulfonylaminoalkyl, arylsulfonylamino, arylsulonylaminoalkyl, heterocyclylsulfonylamino, heterocyclylsulfonylaminoalkyl, heterocyclyl, aryl, or other groups specified.

“Deuterated C₁₋₆alkoxy” is an alkoxy group as defined herein wherein one or more hydrogen atom is replaced with a deuterium as defined herein. In some embodiments, the deuterated C₁₋₆alkoxy comprises one deuterium. In other embodiments, the deuterated C₁₋₆alkoxy comprises two deuterium. In further embodiments, the deuterated C₁₋₆alkoxy comprises three deuterium. Specific examples of deuterated C₁₋₆alkoxy include, without limitation, —OCH₂D, —OCHD₂, —OCD₃, —OCH₂CH₂D, —OCH₂CHD₂, —OCH₂CD₃, —OCHDCH₃, —OCD₂CH₃, among others.

“Halo” as used herein refers to a halogen substituent. In some embodiments, the halo is F, Cl, Br, or I. In other embodiments, the halo is F. In further embodiments, the halo is Cl. In further embodiments, the halo is Br. In still other embodiments, the halo is I.

“C₁₋₆haloalkyl” refers to a group where the point of attachment is through the alkyl group, the alkyl is defined herein, and one or more carbon atom is substituted with one or more halo. Examples of C₁₋₆haloalkyl include C₁₋₆fluoroalkyl, C₁₋₆chloroalkyl, C₁₋₆bromoalkyl, or C₁₋₆iodooalkyl. In some embodiments, the C₁₋₆haloalkyl comprises one halo. In other embodiments, the C₁₋₆haloalkyl comprises two halo. In further embodiments, the C₁₋₆haloalkyl comprises three halo. Specific examples of C₁₋₆haloalkyl include, without limitation, —CH₂F, —CHF₂, —CF₃, —CH₂CH₂F, —CH₂CHF₂, —CH₂CF₃, —CHFCH₃, —CF₂CH₃, among others.

“C₁₋₆haloalkoxy” refers to a group where the point of attachment is through the alkoxy group, the alkoxy is defined herein, and one or more carbon atom is substituted with one or more halo. Examples of C₁₋₆haloalkoxy include C₁₋₆fluoroalkoxy, C₁₋₆chloroalkoxy, C₁₋₆bromoalkoxy, or C₁₋₆iodooalkoxy. In some embodiments, the C₁₋₆haloalkoxy comprises one halo. In other embodiments, the C₁₋₆haloalkoxy comprises two halo. In further embodiments, the C₁₋₆haloalkoxy comprises three halo. Specific examples of C₁₋₆haloalkoxy include, without limitation, —OCH₂F, —OCHF₂, —OCF₃, —OCH₂CH₂F, —OCH₂CHF₂, —OCH₂CF₃, —OCHFCH₃, —OCF₂CH₃, among others.

“—C₀₋₆alkyl-C₃₋₁₂carbocyclyl” refers to a group where the point of attachment is through the alkyl group (if present) and the alkyl and carbocyclyl groups are defined herein. In some embodiments, —C₀₋₆alkyl-C₃₋₁₂carbocyclyl refers to a —C₃₋₁₂carbocyclyl group lacking carbon atoms. In other embodiments, —C₀₋₆alkyl-C₃₋₁₂carbocyclyl refers to —C₁₋₆alkyl-C₃₋₁₂carbocyclyl.

“—C₀₋₆alkyl-C₆₋₁₄aryl” refers to a group where the point of attachment is through the alkyl group (if present) and the alkyl and aryl groups are defined herein. In some embodiments, —C₀₋₆alkyl-C₆₋₁₄aryl refers to a C₆₋₁₄aryl group lacking carbon atoms. In other embodiments-C₀₋₆alkyl-C₆₋₁₄aryl refers to —C₁₋₆alkyl-C₆₋₁₄aryl.

“—C₀₋₆alkyl-O—C₆₋₁₄aryl” refers to a group where the point of attachment is through the alkyl group (if present) and the alkyl and aryl groups are defined herein. In some embodiments, —C₀₋₆alkyl-O—C₆₋₁₄aryl refers to a —O—C₆₋₁₄aryl group lacking carbon atoms. In other embodiments, —C₀₋₆alkyl-O—C₆₋₁₄aryl refers to —C₁₋₆alkyl-O—C₆₋₁₄aryl.

“—C₀₋₆alkyl-(3- to 14-membered heteroaryl)” refers to a group where the point of attachment is through the alkyl group (if present) and the alkyl and heteroaryl groups are defined herein. In some embodiments, —C₀₋₆alkyl-(3- to 14-membered heteroaryl) refers to a 3- to 14-membered heteroaryl. In other embodiments, —C₁₋₆alkyl-(3- to 14-membered heteroaryl).

“—C₀₋₆alkyl-(3- to 14-membered heterocyclyl)” refers to a group where the point of attachment is through the alkyl group (if present) and the alkyl and heterocyclyl groups are defined herein. In some embodiments, —C₀₋₆alkyl-(3- to 14-membered heterocyclyl) refers to a 3- to 14-membered heterocyclyl. In other embodiments, —C₁₋₆alkyl-(3- to 14-membered heterocyclyl).

“—C₀₋₆alkyl-O-(3- to 14-membered heterocyclyl)” refers to a group where the point of attachment is through the alkyl group (if present) and the alkyl and heterocyclyl groups are defined herein. In some embodiments, —C₀₋₆alkyl-O-(3- to 14-membered heterocyclyl) refers to —O-(3- to 14-membered heterocyclyl). In other embodiments, —C₁₋₆alkyl-O-(3- to 14-membered heterocyclyl).

“—S(O)₀₋₂NHC₁₋₆alkyl” refers to a group where the point of attachment is through the sulfur atom and the alkyl group is defined herein. In some embodiments, the —S(O)₀₋₂NHC₁₋₆alkyl is —S—NHC₁₋₆alkyl. In other embodiments, the —S(O)₀₋₂NHC₁₋₆alkyl is a —S(O)NHC₁₋₆alkyl. In further embodiments, the —S(O)₀₋₂NHC₁₋₆alkyl is —S(O)₂NHC₁₋₆alkyl.

“—S(O)₀₋₂(C₁₋₆alkyl)” refers to a group where the point of attachment is through the sulfur atom and the alkyl group is defined herein. In some embodiments, the —S(O)₀₋₂(C₁₋₆alkyl) is —S—(C₁₋₆alkyl). In other embodiments, the —S(O)₀₋₂(C₁₋₆alkyl) is a —S(O)(C₁₋₆alkyl). In further embodiments, the —S(O)₀₋₂(C₁₋₆alkyl) is —S(O)₂(C₁₋₆alkyl).

“—S(O)₀₋₂(C₁₋₆alkyl)₂” refers to a group where the point of attachment is through the sulfur atom and the alkyl group is defined herein. In some embodiments, the —S(O)₀₋₂(C₁₋₆alkyl)₂ is —S—(C₁₋₆alkyl)₂. In other embodiments, the —S(O)₀₋₂(C₁₋₆alkyl)₂ is a —S(O)(C₁₋₆alkyl)₂. In further embodiments, the —S(O)₀₋₂(C₁₋₆alkyl)₂ is —S(O)₂(C₁₋₆alkyl)₂.

“—S(O)₀₋₂(C₆₋₁₄aryl)” refers to a group where the point of attachment is through the sulfur atom and the aryl group is defined herein. In some embodiments, the —S(O)₀₋₂(aryl) is —S-(aryl). In other embodiments, the —S(O)₀₋₂(aryl) is a —S(O)(aryl). In further embodiments, the —S(O)₀₋₂(aryl) is —S(O)₂(aryl).

“—S(O)₀₋₂(heteroaryl)” refers to a group where the point of attachment is through the sulfur atom and the heteroaryl group is defined herein. In some embodiments, the —S(O)₀₋₂(heteroaryl) is —S-(heteroaryl). In other embodiments, the —S(O)₀₋₂(heteroaryl) is a —S(O)(heteroaryl). In further embodiments, the —S(O)₀₋₂(heteroaryl) is —S(O)₂(heteroaryl).

“—S(O)₀₋₂(heterocyclyl)” refers to a group where the point of attachment is through the sulfur atom and the heterocyclyl group is defined herein. In some embodiments, the —S(O)₀₋₂(heterocyclyl) is —S-(heterocyclyl). In other embodiments, the —S(O)₀₋₂(heterocyclyl) is a —S(O)(heterocyclyl). In further embodiments, the —S(O)₀₋₂(heterocyclyl) is a —S(O)₂(heterocyclyl).

“—C(O)(C₁₋₆alkyl)” refers to a group where the point of attachment is through the carbon atom and the alkyl group is defined herein.

“—C(O)(C₃₋₁₄carbocyclyl)” refers to a group where the point of attachment is through the carbon atom and the carbocyclyl group is defined herein.

“—C(O)(C₆₋₁₄aryl)” refers to a group where the point of attachment is through the carbon atom of the carbonyl and the aryl group is defined herein.

“C(O)(heteroaryl)” refers to a group where the point of attachment is through the carbon atom of the carbonyl and the heteroaryl group is defined herein.

“—C(O)(heterocyclyl)” refers to a group where the point of attachment is through the carbon atom of the carbonyl and the heterocyclyl group is defined herein.

“—C(O)NHC₁₋₆alkyl” refers to a group where the point of attachment is through the carbon atom and the alkyl group is defined herein.

“—C(O)NH(C₁₋₆alkyl)₂” refers to a group where the point of attachment is through the carbon atom and the alkyl group is defined herein. The alkyl groups may be the same or differ.

“—C₀₋₆alkyl-NH₂” refers to a group where the point of attachment is through the alkyl group (if present) and the alkyl group is defined herein. In some embodiments, —C₀₋₆alkyl-NH₂ refers to a —NH₂ group. In other embodiments, —C₀₋₆alkyl-NH₂ refers to —C₁₋₆alkyl-NH₂.

“—C₀₋₆alkyl-NH(alkyl)” refers to a group where the point of attachment is through the alkyl group (if present) and the alkyl group is defined herein. In some embodiments, —C₀₋₆alkyl-NH(alkyl) refers to a —NH(alkyl) group. In other embodiments, —C₀₋₆alkyl-NH(alkyl) refers to —C₁₋₆alkyl-NH(alkyl).

“—C₀₋₆alkyl-N(alkyl)₂” refers to a group where the point of attachment is through the alkyl group (if present) and the alkyl group is defined herein. In some embodiments, —C₀₋₆alkyl-N(alkyl)₂ refers to a —N(alkyl)₂ group. In other embodiments, —C₀₋₆alkyl-N(alkyl)₂ refers to —C₁₋₆alkyl-N(alkyl)₂. The alkyl groups may be the same or differ.

“Optionally substituted” unless otherwise specified means that a group may be unsubstituted or substituted by one or more (e.g. 1, 2, 3 or 4) of the substituents listed for that group in which said substituents may be the same or different. In some embodiments, an optionally substituted group has 1 substituent. In other embodiments, an optionally substituted group has 2 substituents. In further embodiments, an optionally substituted group has 3 or more substituents.

“Pharmaceutically acceptable” as used herein refers to approved or approvable by a regulatory agency of the Federal or a state government or the corresponding agency in countries other than the United States, or that is listed in the U.S. Pharmacopoeia or other generally recognized pharmacopoeia for use in animals, such as humans.

The terms “patient” or “subject” refer to mammalian animals and are interchangeable. In some embodiments, the patient or subject is a human. In some embodiments the patient or subject is an adult. In other embodiments the patient or subject is a child. In further embodiments, the patient or subject is a veterinary or farm animal, a domestic animal or pet, or animal normally used for clinical research.

“Treating” any disease or disorder refers, in some embodiments, ameliorating at least one physical parameter, which may not be discernible by the subject. In other embodiments, “treating” refers to modulating the disease or disorder. In further embodiments “treating” refers to physically modulating the disease or disorder, i.e., stabilizing a discernible symptom. In yet other embodiments, “treating” refers to physiologically modulating the disease or disorder, i.e., stabilizing a physical parameter. In still further embodiments, “treating” refers to physically and physiologically modulating the disease or disorder. In other embodiments, “treating” refers to delaying the onset of the disease or disorder.

“Preventing” any disease or disorder refers, in some embodiments, to ameliorating the disease or disorder (i.e., arresting or reducing the development of the disease or at least one of the clinical symptoms thereof).

“Compounds of the application” or variations thereof are meant to embrace compounds of the Formulae (I)-(XIII) as described herein. Also included in “compositions of the application” are pharmaceutically acceptable salts, as applicable. Similarly, reference to intermediates, whether or not claimed, is meant to embrace their salts, and solvates, where the context so permits.

### Compounds

The present application provides compounds of Formula I, or pharmaceutically acceptable salts thereof.

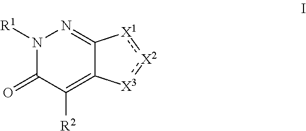

R¹ is C₆₋₁₄-aryl or 3- to 14-membered heteroaryl, wherein each is optionally substituted, as permitted, by one or more of R^{A}.

In some aspects, R¹ is optionally substituted C₆₋₁₄-aryl. In some embodiments, R¹ is optionally substituted C₆₋₁₀aryl. In other embodiments, R¹ is optionally substituted C₆₋₈aryl. In further embodiments, R¹ is optionally substituted phenyl or optionally substituted naphthyl. In yet other embodiments, R¹ is optionally substituted phenyl. In still further embodiments, R¹ is:

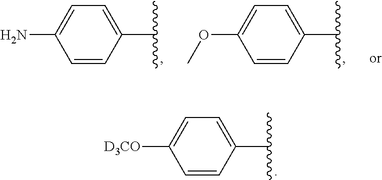

In other aspects, R¹ is optionally substituted 3- to 14-membered heteroaryl. The optionally substituted 3- to 14-membered heteroaryl comprises O, S, and/or N-atoms. In some embodiments, optionally substituted 3- to 14-membered heteroaryl comprises at least one N. In other embodiments, optionally substituted 3- to 14-membered heteroaryl comprises one N and one S. In further embodiments, the optionally substituted 3- to 14-membered heteroaryl comprises two N. In yet other embodiments, the optionally substituted 3- to 14-membered heteroaryl comprises one N and one O. In some embodiments, R¹ is optionally substituted 5- to 12-membered heteroaryl. In other embodiments, R¹ is optionally substituted 6- to 10-membered heteroaryl. In further embodiments, R¹ is optionally substituted 6- to 8-membered heteroaryl. In yet other embodiments, the optionally substituted 3- to 14-membered heteroaryl is thienyl, furyl, imidazolyl, pyrazolyl, thiazolyl, isothiazolyl, oxazolyl, isoxazolyl, triazolyl, thiadiazolyl, oxadiazolyl, tetrazolyl, thiatriazolyl, oxatriazolyl, pyridyl, pyrimidyl, pyrazinyl, pyridazinyl, thiazinyl, oxazinyl, triazinyl, thiadiazinyl, oxadiazinyl, dithiazinyl, dioxazinyl, oxathiazinyl, tetrazinyl, thiatriazinyl, oxatriazinyl, dithiadiazinyl, imidazolinyl, dihydropyrimidyl, tetrahydropyrimidyl, tetrahydropyridinyl, tetrazolopyridazinyl, purinyl, benzoxazolyl benzoxazinyl, benzofuryl, benzothiazolyl, benzothiadiazolyl, benzotriazolyl, benzoimidazolyl, indolyl, or quinolinyl. In still further embodiments, the optionally substituted 3- to 14-membered heteroaryl is pyridyl, benzothiazolyl, quinolinyl, pyrazolyl, benzoxazinyl such as benzo[1,4]oxazinyl, tetrahydropyridinyl such as 1,2,3,6-tetrahydropyridin-yl, or benzimidazolyl such as benzo[d]imidazolyl. In other embodiments, R¹ is:

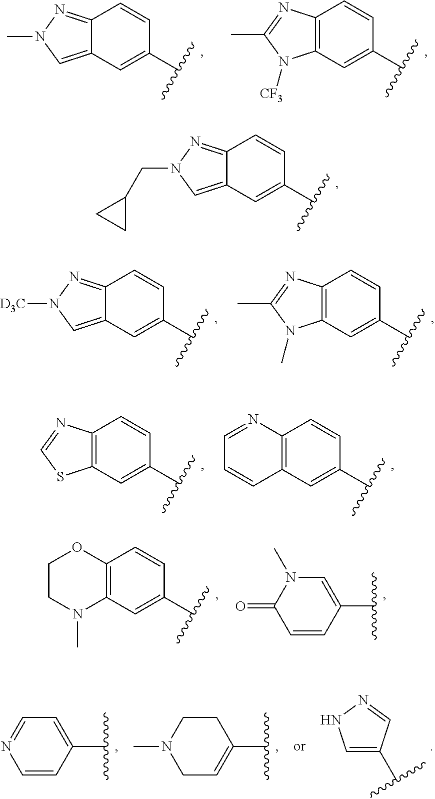

R¹ is optionally substituted at any position on the molecule that results in a stable chemical structure. In some embodiments, the R¹ substituent is bound to a carbon atom of the aryl or heteroaryl group. In other embodiments, the R¹ substituent is bound to a heteroatom of the heteroaryl. In yet other embodiments, R¹ is substituted with R^{A} as defined herein. In other embodiments, R¹ is substituted with one or more of C₁₋₆alkyl, such as methyl, ethyl, propyl, butyl, pentyl, or hexyl, or such as methyl; deuterated C₁₋₆alkyl, such as CH₂D, CHD₂, or CD₃, or such as CD₃; C₁₋₆haloalkyl, such as C₁₋₆fluoroalkyl, or such as CF₃, CHF₂, CH₂F, or such as CF₃; NH₂; C₁₋₆alkoxy, such as methoxy, ethoxy, propoxy, butoxy, pentoxy, or hexoxy, or such as methoxy; —C₀₋₆alkyl-C₃₋₈carbocylyl, such as —C₀₋₆alkyl-C₃₋₈cycloalkyl or —CH₂—C₃₋₈cycloalkyl or C₀₋₆alkyl-cyclopropyl, or such as CH₂-cyclopropyl, CH₂-cyclobutyl, CH₂-cyclopentyl, CH₂-cyclohexyl, or such as CH₂-cyclopropyl, or such as CH₂CH₂-cyclopropyl. In further embodiments, R¹ is substituted with C₁₋₆alkyl. In yet other embodiments, R¹ is substituted with methyl, ethyl, propyl, butyl, pentyl, or hexyl. In still further embodiments, R¹ is substituted with methyl. In other embodiments, R¹ is substituted with deuterated C₁₋₆alkyl. In further embodiments, R¹ is substituted with CH₂D, CHD₂, or CD₃. In still other embodiments, R¹ is substituted with CD₃. In yet further embodiments, R¹ is substituted with C₁₋₆haloalkyl. In other embodiments, R¹ is substituted with C₁₋₆fluoroalkyl. In further embodiments, R¹ is substituted with CF₃, CHF₂, CH₂F. In yet other embodiments, R¹ is substituted with CF₃. In still further embodiments, R¹ is substituted with NH₂. In other embodiments, R¹ is substituted with C₁₋₆alkoxy. In further embodiments, R¹ is substituted with methoxy, ethoxy, propoxy, butoxy, pentoxy, or hexoxy. In still other embodiments, R¹ is substituted with methoxy. In yet further embodiments, R¹ is substituted with —C₀₋₆alkyl-C₃₋₈carbocylyl. In other embodiments, R¹ is substituted with —C₀₋₆alkyl-C₃₋₈cycloalkyl. In further embodiments, R¹ is substituted with —CH₂—C₃₋₈cycloalkyl. In yet other embodiments, R¹ is substituted with C₀₋₆alkyl-cyclopropyl. In still further embodiments, R¹ is substituted with CH₂-cyclopropyl, CH₂-cyclobutyl, CH₂-cyclopentyl, CH₂-cyclohexyl. In other embodiments, R¹ is substituted with CH₂-cyclopropyl. In further embodiments, R¹ is substituted with CH₂CH₂-cyclopropyl.

R² is —C₀₋₆alkyl-C₆₋₁₄aryl or —C₀₋₆alkyl-(3- to 14-membered heteroaryl), wherein each, as permitted, is optionally substituted by one or more of R^{A}. In some aspects, R² is —C₀₋₆alkyl-optionally substituted C₆₋₁₄-aryl. In some embodiments, R² is —C₀₋₆alkyl-optionally substituted C₆₋₁₄-aryl. In other embodiments, R² is optionally substituted C₆₋₁₀aryl. In further embodiments, R² is optionally substituted C₈₋₁₀aryl. In yet other embodiments, R² is optionally substituted phenyl or naphthyl. In still further embodiments, R² is optionally substituted phenyl. In other embodiments, R² is optionally substituted naphthyl. In further embodiments, R² is:

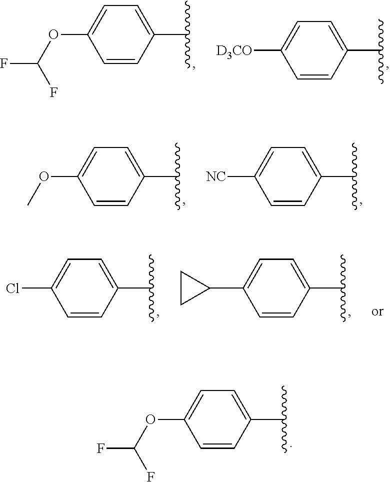

In other aspects, R² is —C₀₋₆alkyl-(optionally substituted 3- to 14-membered heteroaryl). In some embodiments, R² is optionally substituted 3- to 14-membered heteroaryl. In other embodiments, R² is optionally substituted 5- to 12-membered heteroaryl. In further embodiments, R² is optionally substituted 6- to 10-membered heteroaryl. The optionally substituted heteroaryl comprises at least one N, S, or O atom. In some embodiments, the optionally substituted heteroaryl comprises at least one N. In other embodiments, the optionally substituted heteroaryl comprises one N and one S. In further embodiments, the optionally substituted heteroaryl comprises two N. In yet other embodiments, the optionally substituted heteroaryl comprises one N and one O. In still further embodiments, the —C₀₋₆alkyl-optionally substituted heteroaryl is thienyl, furyl, imidazolyl, pyrazolyl, thiazolyl, isothiazolyl, oxazolyl, isoxazolyl, triazolyl, thiadiazolyl, oxadiazolyl, tetrazolyl, thiatriazolyl, oxatriazolyl, pyridyl, pyrimidyl, pyrazinyl, pyridazinyl, thiazinyl, oxazinyl, triazinyl, thiadiazinyl, oxadiazinyl, dithiazinyl, dioxazinyl, oxathiazinyl, tetrazinyl, thiatriazinyl, oxatriazinyl, dithiadiazinyl, imidazolinyl, dihydropyrimidyl, tetrahydropyrimidyl, tetrahydropyridinyl, tetrazolopyridazinyl, purinyl, benzoxazolyl benzoxazinyl, benzofuryl, benzothiazolyl, benzothiadiazolyl, benzotriazolyl, benzoimidazolyl, indolyl, or quinolinyl. In other embodiments, the —C₀₋₆alkyl-optionally substituted heteroaryl is pyridyl, benzothiazolyl, quinolinyl, pyrazolyl, benzoxazinyl, tetrahydropyridinyl, or benzimidazolyl. In further embodiments, the —C₀₋₆alkyl-optionally substituted heteroaryl is benzo[1,4]oxazinyl. In yet other embodiments, the —C₀₋₆alkyl-optionally substituted heteroaryl is 1,2,3,6-tetrahydropyridin-yl. In still further embodiments, the —C₀₋₆alkyl-optionally substituted heteroaryl is benzo[d]imidazolyl. In yet other embodiments, the —C₀₋₆alkyl-optionally substituted heteroaryl pyridyl. In still further embodiments, R² is:

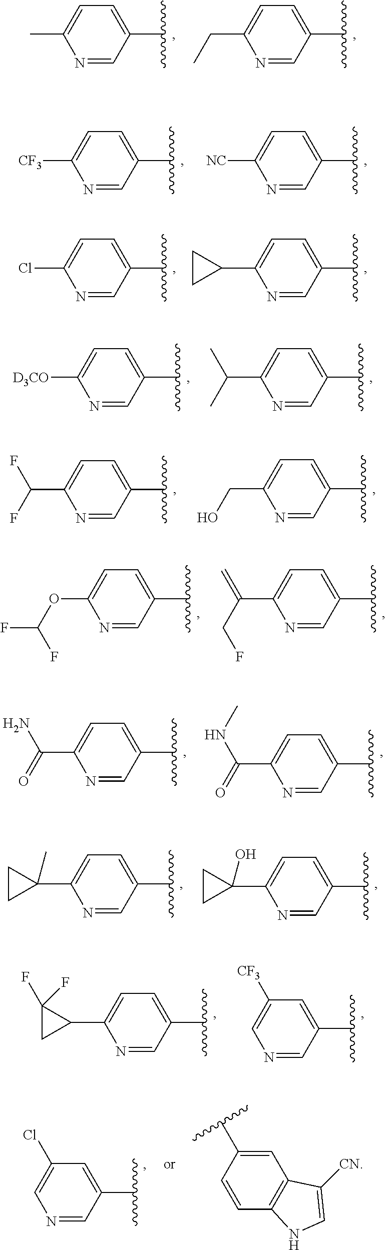

R² is optionally substituted at any position on the molecule that results in a stable chemical structure. In some embodiments, the R² substituent is bound to a carbon atom of the alkyl, aryl or heteroaryl group. In other embodiments, the R² substituent is bound to a heteroatom of the heteroaryl. In yet other embodiments, R² is substituted with R^{A} as defined herein. In still further embodiments, R² is substituted with one or more of halo, such as F, Cl, Br, or I, or such as F or Cl, or such as C₁; CN; C₁₋₆alkyl, such as methyl, ethyl, propyl, butyl, pentyl, or hexyl, or such as methyl; deuterated C₁₋₆alkyl, such as CH₂D, CHD₂, or CD₃, or such as CD₃; C₁₋₆haloalkyl, such as C₁₋₆fluoroalkyl, or such as CF₃, CHF₂, or CH₂F, or such as CF₃; C₁₋₆alkylOH, such as CH₂OH, CH₂CH₂OH, propyl-OH, butyl-OH, pentyl-OH, or hexyl-OH, or such as CH₂OH; C₁₋₆alkoxy such as methoxy, ethoxy, propoxy, butoxy, pentoxy, or hexoxy, or such as methoxy; C₁₋₆haloalkoxy, such as C₁₋₆fluoroalkoxy, or such as OCF₃, OCHF₂, or OCH₂F, or such as OCF₃; deuterated C₁₋₆alkoxy, such as OCH₂D, OCHD₂, or OCD₃, or such as OCD₃; C₂₋₆haloalkenyl, such as C₂₋₆fluoroalkenyl, or such as fluoroethenyl, fluoropropenyl, fluorobutenyl, fluoropentenyl, or fluorohexenyl, or such as fluoropropenyl; —C₀₋₆alkyl-C₃₋₁₀carbocyclyl, such as C₃₋₁₀carbocyclyl, such as C₃₋₁₀cycloalkyl or C₃₋₈cycloalkyl or C₃₋₆cycloalkyl, such as cyclopropyl, cyclobutyl, cyclopentyl, cyclohexyl, or such as cyclopropyl; C(O)NH₂; or C(O)NHC₁₋₆alkyl, such as C(O)NHmethyl, C(O)NHethyl, C(O)NHpropyl, C(O)NHbutyl, C(O)NHpentyl, or C(O)NHhexyl, or such as C(O)NHmethyl. In other embodiments, R² is substituted with halo such as F, Cl, Br, or I, or such as F or Cl, or such as C₁. In other embodiments, R² is substituted with CN. In further embodiments, R² is substituted with C₁₋₆alkyl such as methyl, ethyl, propyl, butyl, pentyl, or hexyl, or such as methyl. In still other embodiments, R² is substituted with deuterated C₁₋₆alkyl such as CH₂D, CHD₂, or CD₃, or such as CD₃. In further embodiments, R² is substituted with C₁₋₆haloalkyl such as C₁₋₆fluoroalkyl, such as CF₃, CHF₂, or CH₂F, or such as CF₃. In further embodiments, R² is substituted with C₁₋₆alkylOH such as CH₂OH, CH₂CH₂OH, propyl-OH, butyl-OH, pentyl-OH, or hexyl-OH, or such as CH₂OH. In other embodiments, R² is substituted with C₁₋₆alkoxy such as methoxy, ethoxy, propoxy, butoxy, pentoxy, or hexoxy, or such as methoxy. In still further embodiments, R² is substituted with C₁₋₆haloalkoxy such as C₁₋₆fluoroalkoxy, or such as OCF₃, OCHF₂, OCH₂F, or such as OCF₃. In yet further embodiments, R² is substituted with deuterated C₁₋₆alkoxy such as OCH₂D, OCHD₂, or OCD₃, or such as such as OCD₃. In still other embodiments, R² is substituted with C₂₋₆haloalkenyl, such as C₂₋₆fluoroalkenyl, or such as fluoroethenyl, fluoropropenyl, fluorobutenyl, fluoropentenyl, or fluorohexenyl, or such as fluoropropenyl. In yet other embodiments, R² is substituted with —C₀₋₆alkyl-C₃₋₁₀carbocyclyl such as C₃₋₁₀carbocyclyl, or such as C₃₋₁₀cycloalkyl, or such as C₃₋₈cycloalkyl, or such as C₃₋₆cycloalkyl, or such as cyclopropyl, cyclobutyl, cyclopentyl, or cyclohexyl, or such as cyclopropyl. In yet further embodiments, R² is substituted with C(O)NH₂. In other embodiments, R² is substituted with C(O)NHC₁₋₆alkyl such as C(O)NHmethyl, C(O)NHethyl, C(O)NHpropyl, C(O)NHbutyl, C(O)NHpentyl, or C(O)NHhexyl, or such as C(O)NHmethyl.

In further aspects, the R² substituent may itself be substituted. In some embodiments, the R² substituent is cyclopropyl and the cyclopropyl group is substituted. In other embodiments, the R² substituent is substituted with one or more of CN; halo such as F, Cl, Br, or I, or such as F or Cl, or such as Cl; OH; or C₁₋₆alkyl such as methyl, ethyl, propyl, butyl, pentyl, or hexyl, or such as methyl. In further embodiments, the R² substituted is substituted with CN. In yet other embodiments, the R² cyclopropyl substituent is substituted with CN. In still further embodiments, the R² substituent is substituted with halo. In other embodiments, the R² substituent is substituted with F, Cl, Br, or I, such as F or Cl, or such as Cl. In still further embodiments, the R² substituent is substituted with OH. In other embodiments, the R² substituent is substituted with C₁₋₆alkyl such as methyl, ethyl, propyl, butyl, pentyl, or hexyl, or such as methyl.

R³ is H, D, CN, halo, OH, C₁₋₆alkyl, C₁₋₆alkylOH, deuterated C₁₋₆alkyl, C₂₋₆alkenyl, C₂₋₆haloalkenyl, C₂₋₆alkynyl, C₁₋₆alkoxy, deuterated C₁₋₆alkoxy, C₁₋₆haloalkyl, —C₀₋₆alkyl-C₃₋₁₂carbocyclyl, —C₀₋₆alkyl-C₆₋₁₄aryl, —C₀₋₆alkyl-O—C₆₋₁₄aryl, —C₀₋₆alkyl-(3- to 14-membered heterocyclyl), —C₀₋₆alkyl-O-(3- to 14-membered heterocyclyl), —C₀₋₆alkyl-(3- to 14-membered heteroaryl), —C₀₋₆alkyl-NR^{A}(CH₂)₀₋₆C(O)R^{A}, NR^{B}R^{C}, C(O)NR^{B}R^{C}, NR^{B}C(NR^{C})NR^{B}R^{C}, NR^{B}C(NR^{C})(═NR^{B}), —S(O)₀₋₂(C₁₋₆alkyl), —S(O)₀₋₂NH₂, —S(O)₀₋₂NHC₁₋₆alkyl, —S(O)₀₋₂(C₁₋₆alkyl)₂, —S(O)₀₋₂(C₆₋₁₄aryl), —S(O)₀₋₂(heteroaryl), —S(O)₀₋₂(heterocyclyl), —C(O)(C₁₋₆alkyl), —C(O)(C₃₋₁₄carbocyclyl), —C(O)NR^{A}R^{B}, —C(O)(C₆₋₁₄aryl), —C(O)(heteroaryl), —C(O)(heterocyclyl), C₁₋₆haloalkoxy, or SR^{B} wherein each of the forgoing, as permitted, is optionally substituted by one or more of R^{A}. In some embodiments, R³ is H. In other embodiments, R³ is C₁₋₆alkyl such as methyl, ethyl, propyl, butyl, pentyl, or hexyl, or such as methyl, or such as ethyl, or such as propyl, or such as n-propyl or i-propyl, or such as butyl, or such as n-butyl, i-butyl, or t-butyl, or such as i-butyl. In further embodiments, R³ is C₁₋₆haloalkyl, such as C₁₋₆fluoroalkyl, or such as CF₃, CHF₂, CH₂F, CH₂CF₃, CH₂CF₂H, CH₂CH₂F, or CH₂CH₂CF₃, or such as CF₃, or such as CH₂CF₃, or such as CH₂CH₂CF₃. In other embodiments, R³ is —C₀₋₆alkyl-C₃₋₈carbocyclyl. In yet other embodiments, R³ is —C₁₋₆alkyl-C₃₋₈cycloalkyl, such as —CH₂—C₃₋₈cycloalkyl, or such as CH₂-cyclopropyl, CH₂-cyclobutyl, CH₂-cyclopentyl, or CH₂-cyclohexyl, or such as CH₂-cyclopropyl, or such as CH₂-cyclobutyl. In further embodiments, R³ is —C₁₋₆alkyl-cyclopropyl. In yet further embodiments, R³ is —C₀₋₆alkyl-C₃₋₈carbocyclyl. In other embodiments, R³ is —C₀₋₆alkyl-C₃₋₈carbocyclyl and the —C₀₋₆alkyl-C₃₋₈carbocyclyl is substituted with one or more of halo, such as F, Cl, Br, or I, or such as F, or such as C₁, or such as Br; or such as I. In further embodiments, R³ is —C₀₋₆alkyl-C₃₋₈carbocyclyl and is substituted with halo, such as F, Cl, Br, or I, or such as F, or such as C₁, or such as Br, or such as I. In further embodiments, R³ is:

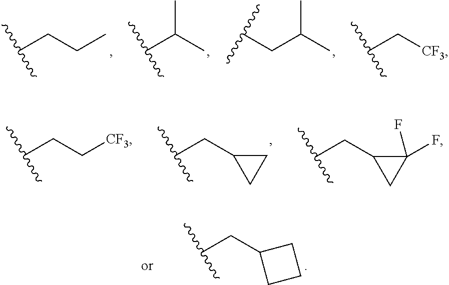

R⁴, R^{4′}, R⁵, and R^{5′} are, independently, H, D, CN, OH, C₁₋₆alkyl, halo, deuterated C₁₋₆alkyl, C₁₋₆alkoxy, deuterated C₁₋₆alkoxy, C₁₋₆alkyl-OH, C₁₋₆haloalkyl, C₁₋₆haloalkoxy, C₂₋₆alkenyl, C₂₋₆haloalkenyl, C₂₋₆alkynyl, —S(O)₀₋₂NH₂, —S(O)₀₋₂NHC₁₋₆alkyl, —S(O)₀₋₂(C₁₋₆alkyl)₂, —S(O)₀₋₂C₁₋₆alkyl, —S(O)₀₋₂(C₆₋₁₄aryl), —S(O)₀₋₂(heteroaryl), —S(O)₀₋₂(heterocyclyl), —C₀₋₆alkyl-(C₃₋₁₄carbocylyl), —C₀₋₆alkyl-C₆₋₁₄aryl, —C₀₋₆alkyl-O—C₆₋₁₄aryl, C₀₋₆alkyl-(3- to 14-membered heterocyclyl), —C₀₋₆alkyl-O-(3- to 14-membered heterocyclyl), C₀₋₆alkyl-(3- to 14-membered heteroaryl), —C₁₋₆alkyl-NR^{A}(CH₂)₀₋₆C(O)R^{A}, NR^{B}R^{C}, C(O)NR^{B}R^{C}, NR^{B}C(NR^{C})NR^{B}R^{C}, NR^{B}C(NR^{C})(═NR^{B}), —C(O)NR^{A}R^{B}, —C(O)(C₁₋₆alkyl), —C(O)(C₃₋₁₄carbocyclyl), —C(O)(heteroaryl), —C(O)(heterocyclyl), —C(O)(C₆₋₁₄aryl), or SR^{B} wherein each of the forgoing, as permitted, is optionally substituted by one or more R^{A}.

In some embodiments, R⁴ is H. In other embodiments, R⁴ is CN. In further embodiments, R⁴ is C₁₋₆alkyl, such as methyl, ethyl, propyl, butyl, pentyl, or hexyl, or such as methyl, or such as ethyl, or such as propyl, or such as n-propyl or i-propyl. In yet other embodiments, R⁴ is halo, such as F, Cl, Br, or I, such as F, or such as Cl, or such as Br, or such as I. In yet other embodiments, R⁴ is C₁₋₆alkylOH such as CH₂OH, CH₂CH₂OH, propyl-OH, butyl-OH, pentyl-OH, or hexyl-OH, or such as CH₂OH, or such as CH(OH)CH₃. In further embodiments, R⁴ is C₁₋₆haloalkyl such as C₁₋₆fluoroalkyl, or such as CF₃, CHF₂, or CH₂F, or such as CF₃. In still other embodiments, R⁴ is C₂₋₆alkynyl such as ethynyl, propynyl, butynyl, pentynyl, or hexynyl, or such as ethynyl. In yet further embodiments, R⁴ is C(O)OH. In other embodiments, R⁴ is C(O)C₁₋₆alkyl such as C(O)methyl, C(O)ethyl, C(O)propyl, C(O)butyl, C(O)pentyl, or C(O)hexyl, or such as C(O)methyl. In further embodiments, R⁴ is C(O)NH₂. In yet other embodiments, R⁴ is C(O)NHC₁₋₆alkyl such as C(O)NHmethyl, C(O)NHethyl, C(O)NHpropyl, C(O)NHbutyl, C(O)NHpentyl, or C(O)NHhexyl, such as C(O)NHmethyl. In still further embodiments, R⁴ is C(O)N(C₁₋₆alkyl)₂ such as C(O)N(methyl)₂, C(O)N(ethyl)₂, C(O)N(propyl)₂, C(O)N(butyl)₂, C(O)N(pentyl)₂, or C(O)N(hexyl)₂, or such as C(O)N(methyl)₂. In other embodiments, R⁴ is S(O)₀₋₂NH₂ such as —S—NH₂, S(O)NH₂, or S(O)₂NH₂, or such as S(O)₂NH₂. In further embodiments, R⁴ is S(O)₀₋₂NHC₁₋₆alkyl such as S(O)₂NHC₁₋₆alkyl, or such as S(O)₂NHmethyl, S(O)₂NHethyl, S(O)₂NHpropyl, S(O)₂NHbutyl, S(O)₂NHpentyl, or S(O)₂NHhexyl, or such as S(O)₂NHmethyl. In yet other embodiments, R⁴ is S(O)₀₋₂N(C₁₋₆alkyl)₂ such as S(O)₂N(C₁₋₆alkyl)₂, or such as S(O)₂N(methyl)₂, S(O)₂N(ethyl)₂, S(O)₂N(propyl)₂, S(O)₂N(butyl)₂, S(O)₂N(pentyl)₂, or S(O)₂N(hexyl)₂, or such as S(O)₂N(methyl)₂. In still further embodiments, R⁴ is S(O)₀₋₂C₁₋₆alkyl such as S(O)₂C₁₋₆alkyl such as S(O)₂methyl, S(O)₂ethyl, S(O)₂propyl, S(O)₂butyl, S(O)₂pentyl, or S(O)₂hexyl, or such as S(O)₂methyl. In other embodiments, R⁴ is C₂₋₆alkynyl such as ethynyl, propynyl, butynyl, pentynyl, or hexynyl.

R⁴ is optionally substituted at any position on the molecule that results in a stable chemical structure. In some embodiments, R⁴ is substituted by Si(alkyl)₃ such as Si(CH₃)₃. In further embodiments, R⁴ is C₂₋₆alkynyl and is substituted by Si(alkyl)₃. In other embodiments, R⁴ is substituted by C₁₋₆haloalkyl such as C₁₋₆fluoroalkyl, or such as CF₃, CHF₂, or CH₂F, or such as CF₃, or such as CHF₂. In further embodiments, R⁴ is substituted by CN. In yet other embodiments, R⁴ is substituted with NHC₁₋₆alkyl such as NHmethyl, NHethyl, or NHpropyl, or such as NHCH₃. In still further embodiments, R⁴ is C₁₋₆alkyl and is substituted with CN. In other embodiments, R⁴ is C₁₋₆alkyl and is substituted with NHC₁₋₆alkyl such as NHmethyl, NHethyl, or NHpropyl, or such as NHCH₃.

In some embodiments, R⁵ is H. In other embodiments, R⁵ is halo such as F, Cl, Br, or I, or such as F or Cl, or such as C₁, or such as F. In further embodiments, R⁵ is C₁₋₆alkyl such as methyl, ethyl, propyl, butyl, pentyl, or hexyl, or such as methyl, or such as ethyl, or such as propyl, e.g., propyl or i-propyl.

In certain embodiments, R⁴ and R^{4′} or R⁵ and R^{5′} are combined to form oxo. In some embodiments, R⁴ and R^{4′} is combined to form oxo. In further embodiments, R⁵ and R^{5′} are combined to form oxo. In other embodiments, R⁴ and R^{4′} and R⁵ and R^{5′} are combined to form oxo.

R⁶ is H or C₁₋₆alkyl. In some embodiments, R⁶ is H. In other embodiments, R⁶ is C₁₋₆alkyl such as methyl, ethyl, propyl, butyl, pentyl, or hexyl, or such as methyl, or such as ethyl, or such as propyl, e.g., n-propyl or i-propyl.

R^{A} is D, CN, NH₂, halo, C₁₋₆alkyl, deuterated C₁₋₆alkyl, C₁₋₆haloalkyl, C₁₋₆alkoxy, C₁₋₆haloalkoxy, deuterated C₁₋₆alkoxy, OH, —C₁₋₆alkylOH, C₂₋₆alkenyl, C₂₋₆haloalkenyl, C₂₋₆alkynyl, —S(O)₀₋₂(C₁₋₆alkyl), —S(O)₀₋₂(C₆₋₁₄aryl), —S(O)₀₋₂NH₂, —S(O)₀₋₂NHC₁₋₆alkyl, —S(O)₀₋₂(C₁₋₆alkyl)₂, —S(O)₀₋₂(heteroaryl), —S(O)₀₋₂(heterocyclyl), —C(O)(C₁₋₆alkyl), —C(O)NH₂, —C(O)NHC₁₋₆alkyl, —C(O)NH(C₁₋₆alkyl)₂, —C(O)(C₃₋₁₄carbocyclyl), —C(O)(heteroaryl), —C(O)(heterocyclyl), —C(O)(C₆₋₁₄aryl), —C₀₋₆alkyl-C₃₋₁₄carbocyclyl, —C₀₋₆alkyl-C₆₋₁₄aryl, —C₀₋₆alkyl-O—C₆₋₁₄aryl, —C₀₋₆alkyl-(3- to 14-membered heterocyclyl), —C₀₋₆alkyl-(3- to 14-membered heteroaryl), or —C₀₋₆alkyl-O-(3- to 14-membered heterocyclyl). In some embodiments, R^{A} is D. In other embodiments, R^{A} is CN. In further embodiments, R^{A} is NH₂. In yet other embodiments, R^{A} is halo. In still further embodiments, R^{A} is C₁₋₆alkyl such as methyl, ethyl, propyl, butyl, pentyl, or hexyl, or such as methyl, or such as ethyl, or such as propyl, or such as butyl. In other embodiments, R^{A} is deuterated C₁₋₆alkyl such as CH₂D, CHD₂, or CD₃, or such as CD₃. In further embodiments, R^{A} is C₁₋₆haloalkyl such as C₁₋₆fluoroalkyl, or such as CF₃, CHF₂, CH₂F, CH₂CF₃, CH₂CF₂H, CH₂CH₂F, or CH₂CH₂CF₃, or such as CF₃, or such as CH₂CF₃, or such as CH₂CH₂CF₃. In yet other embodiments, R^{A} is C₁₋₆alkoxy such as methoxy, ethoxy, propoxy, butoxy, pentoxy, or hexoxy, or such as methoxy. In still further embodiments, R^{A} is C₁₋₆haloalkoxy such as C₁₋₆fluoroalkoxy, or such as OCF₃, OCHF₂, OCH₂F, or such as OCF₃. In other embodiments, R^{A} is deuterated C₁₋₆alkoxy such as OCH₂D, OCHD₂, or OCD₃, or such as OCD₃. In further embodiments, R^{A} is OH. In still other embodiments, R^{A} is —C₁₋₆alkylOH such as CH₂OH, CH₂CH₂OH, propyl-OH, butyl-OH, pentyl-OH, or hexyl-OH, or such as CH₂OH. In yet further embodiments, R^{A} is C₂₋₆alkenyl such as ethenyl, propenyl, butenyl, pentenyl, or hexenyl, or such as propenyl. In other embodiments, R^{A} is C₂₋₆haloalkenyl such as C₂₋₆fluoroalkenyl, or such as fluoroethenyl, fluoropropenyl, fluorobutenyl, fluoropentenyl, or fluorohexenyl, or such as fluoropropenyl. In further embodiments, R^{A} is C₂₋₆alkynyl such as ethynyl, propynyl, butynyl, pentynyl, or hexynyl, or such as propynyl. In yet other embodiments, R^{A} is —S(O)₀₋₂(C₁₋₆alkyl) such as S(O)₂(C₁₋₆alkyl), or such as S(O)₂(methyl), S(O)₂(ethyl), S(O)₂(propyl), S(O)₂(butyl), S(O)₂(pentyl), or S(O)₂(hexyl), or such as S(O)₂(methyl). In still further embodiments, R^{A} is —S(O)₀₋₂(C₆₋₁₄aryl) such as —S—(C₆₋₁₄aryl), —S(O)(C₆₋₁₄aryl), or such as —S(O)₂(C₆₋₁₄aryl), or such as —S-(phenyl), —S(O)(phenyl), or such as —S(O)₂(phenyl). In other embodiments, R^{A} is —S(O)₀₋₂NH₂ or such as —S—NH₂, S(O)NH₂, or S(O)₂NH₂, or such as S(O)₂NH₂. In further embodiments, R^{A} is —S(O)₀₋₂NHC₁₋₆alkyl such as S(O)₂NHC₁₋₆alkyl, or such as S(O)₂NHmethyl, S(O)₂NHethyl, S(O)₂NHpropyl, S(O)₂NHbutyl, S(O)₂NHpentyl, or S(O)₂NHhexyl, or such as S(O)₂NHmethyl. In still other embodiments, R^{A} is —S(O)₀₋₂(C₁₋₆alkyl)₂ or such as S(O)₂(C₁₋₆alkyl)₂, or such as S(O)₂(methyl)₂, S(O)₂(ethyl)₂, S(O)₂(propyl)₂, S(O)₂(butyl)₂, S(O)N(pentyl)₂, or S(O)₂(hexyl)₂, or such as S(O)₂(methyl)₂. In yet further embodiments, R^{A} is —S(O)₀₋₂(heteroaryl) such as —S-(heteroaryl), R^{A} is —S(O)(heteroaryl), or —S(O)₂(heteroaryl). In other embodiments, R^{A} is —S(O)₀₋₂(heterocyclyl) such as —S-(heterocyclyl), R^{A} is —S(O)(heterocyclyl), or —S(O)₂(heterocyclyl). In further embodiments, R^{A} is —C(O)(C₁₋₆alkyl) such as C(O)methyl, C(O)ethyl, C(O)propyl, C(O)butyl, C(O)pentyl, or C(O)hexyl, or such as C(O)methyl. In yet other embodiments, R^{A} is —C(O)NH₂. In still other embodiments, R^{A} is —C(O)NHC₁₋₆alkyl such as C(O)NHmethyl, C(O)NHethyl, C(O)NHpropyl, C(O)NHbutyl, C(O)NHpentyl, or C(O)NHhexyl, or such as C(O)NHmethyl. In yet further embodiments, R^{A} is —C(O)N(C₁₋₆alkyl)₂ such as C(O)N(methyl)₂, C(O)N(ethyl)₂, C(O)N(propyl)₂, C(O)N(butyl)₂, C(O)N(pentyl)₂, or C(O)N(hexyl)₂, or such as C(O)N(methyl)₂. In other embodiments, R^{A} is —C(O)(C₃₋₁₄carbocyclyl) such as —C(O)(cyclopropyl), C(O)(cyclobutyl), C(O)(cyclopentyl), C(O)(cyclohexyl), C(O)(cyclopropenyl), C(O)(cyclobutenyl), C(O)(cyclopentenyl), or C(O)(cyclohexenyl), or such as C(O)(cyclopropyl). In further embodiments, R^{A} is —C(O)(heteroaryl). In yet other embodiments, R^{A} is —C(O)(heterocyclyl). In still further embodiments, R^{A} is —C(O)(C₆₋₁₄aryl) such as —C(O)(phenyl). In other embodiments, R^{A} is —C₀₋₆alkyl-C₃₋₁₄carbocyclyl such as —C₃₋₁₄carbocyclyl or —CH₂—C₃₋₁₄carbocyclyl, or such as cyclopropyl, cyclobutyl, cyclopentyl, cyclohexyl, cyclopropenyl, cyclobutenyl, cyclopentenyl, or cyclohexenyl, or such as —CH₂-cyclopropyl, cyclobutyl, cyclopentyl, cyclohexyl, cyclopropenyl, cyclobutenyl, cyclopentenyl, or cyclohexenyl. In further embodiments, R^{A} is —C₀₋₆alkyl-C₆₋₁₄aryl such as —C₆₋₁₄aryl or CH₂—C₆₋₁₄aryl, or such as phenyl, or such as —CH₂-phenyl. In still other embodiments, R^{A} is —C₀₋₆alkyl-O—C₆₋₁₄aryl such as —O—C₆₋₁₄aryl or CH₂—O—C₆₋₁₄aryl, or such as —O-phenyl, or such as —CH₂—O-phenyl. In yet further embodiments, R^{A} is —C₀₋₆alkyl-(3- to 14-membered heterocyclyl) such as −3- to 14-membered heterocyclyl or CH₂-(3- to 14-membered heterocyclyl). In other embodiments, R^{A} is —C₀₋₆alkyl-(3- to 14-membered heteroaryl) such as -heteroaryl or CH₂-(heteroaryl). In further embodiments, R^{A} is —C₀₋₆alkyl-O-(3- to 14-membered heterocyclyl) such as —O-3- to 14-membered heterocyclyl or CH₂—O-(3- to 14-membered heterocyclyl).

R^{A}, as permitted, is optionally substituted by D, CN, halo, C₁₋₆alkyl, deuterated C₁₋₆alkyl, C₁₋₆haloalkyl, —C₀₋₆alkyl-C₃₋₁₄carbocyclyl, C₁₋₆alkoxy, C₁₋₆haloalkoxy, deuterated C₁₋₆alkoxy, OH, —C₁₋₆alkylOH, C₂₋₆alkenyl, C₂₋₆haloalkenyl, C₂₋₆alkynyl, —S(O)₀₋₂(C₁₋₆alkyl), —S(O)₀₋₂(C₆₋₁₄aryl), —S(O)₀₋₂(heteroaryl), —S(O)₀₋₂(heterocyclyl), —S(O)₀₋₂NH₂, —S(O)₀₋₂NHC₁₋₆alkyl, —S(O)₀₋₂(C₁₋₆alkyl)₂, —C(O)(C₁₋₆alkyl), —C(O)NH₂, —C(O)NHC₁₋₆alkyl, —C(O)NH(C₁₋₆alkyl)₂, —C(O)(C₃₋₁₄carbocyclyl), —C(O)(C₆₋₁₄aryl), —C(O)(heteroaryl), —C(O)(heterocyclyl), —C₀₋₆alkyl-O—C₆₋₁₄aryl, —C₀₋₆alkyl-C₆₋₁₄aryl, —C₀₋₆alkyl-(3- to 14-membered heterocyclyl), —C₀₋₆alkyl-(3- to 14-membered heteroaryl), —C₀₋₆alkyl-O-(3- to 14-membered heterocyclyl), —C₀₋₆alkyl-NH₂, —C₀₋₆alkyl-NH(alkyl), or —C₀₋₆alkyl-N(alkyl)₂. In some embodiments, R^{A} is substituted by D. In other embodiments, R^{A} is substituted by CN. In yet other embodiments, R^{A} is substituted by halo. In still further embodiments, R^{A} is substituted by C₁₋₆alkyl such as methyl, ethyl, propyl, butyl, pentyl, or hexyl, or such as methyl, or such as ethyl, or such as propyl, or such as butyl. In other embodiments, R^{A} is substituted by deuterated C₁₋₆alkyl such as CH₂D, CHD₂, or CD₃, or such as CD₃. In further embodiments, R^{A} is substituted by C₁₋₆haloalkyl such as C₁₋₆fluoroalkyl, or such as CF₃, CHF₂, CH₂F, CH₂CF₃, CH₂CF₂H, CH₂CH₂F, or CH₂CH₂CF₃, or such as CF₃, or such as CH₂CF₃, or such as CH₂CH₂CF₃. In yet other embodiments, R^{A} is substituted by C₁₋₆alkoxy such as methoxy, ethoxy, propoxy, butoxy, pentoxy, or hexoxy, or such as methoxy. In still further embodiments, R^{A} is substituted by C₁₋₆haloalkoxy such as C₁₋₆fluoroalkoxy, such as OCF₃, OCHF₂, OCH₂F, or such as OCF₃. In other embodiments, R^{A} is substituted by deuterated C₁₋₆alkoxy such as OCH₂D, OCHD₂, or OCD₃, such as OCD₃. In further embodiments, R^{A} is substituted by OH. In still other embodiments, R^{A} is substituted by —C₁₋₆alkylOH such as CH₂OH, CH₂CH₂OH, propyl-OH, butyl-OH, pentyl-OH, or hexyl-OH, or such as CH₂OH. In yet further embodiments, R^{A} is substituted by C₂₋₆alkenyl such as ethenyl, propenyl, butenyl, pentenyl, or hexenyl, or such as propenyl. In other embodiments, R^{A} is substituted by C₂₋₆haloalkenyl such as C₂₋₆fluoroalkenyl, or such as fluoroethenyl, fluoropropenyl, fluorobutenyl, fluoropentenyl, or fluorohexenyl, or such as fluoropropenyl. In further embodiments, R^{A} is substituted by C₂₋₆alkynyl such as ethynyl, propynyl, butynyl, pentynyl, or hexynyl, or such as propynyl. In yet other embodiments, R^{A} is substituted by —S(O)₀₋₂(C₁₋₆alkyl) such as S(O)₂(C₁₋₆alkyl), or such as S(O)₂(methyl), S(O)₂(ethyl), S(O)₂(propyl), S(O)₂(butyl), S(O)₂(pentyl), or S(O)₂(hexyl), or such as S(O)₂(methyl). In still further embodiments, R^{A} is substituted by —S(O)₀₋₂(C₆₋₁₄aryl) such as —S—(C₆₋₁₄aryl), —S(O)(C₆₋₁₄aryl), or —S(O)₂(C₆₋₁₄aryl), or such as —S-(phenyl), —S(O)(phenyl), or —S(O)₂(phenyl). In other embodiments, R^{A} is substituted by —S(O)₀₋₂NH₂ such as —S—NH₂, S(O)NH₂, or S(O)₂NH₂, or such as S(O)₂NH₂. In further embodiments, R^{A} is substituted by —S(O)₀₋₂NHC₁₋₆alkyl such as S(O)₂NHC₁₋₆alkyl, or such as S(O)₂NHmethyl, S(O)₂NHethyl, S(O)₂NHpropyl, S(O)₂NHbutyl, S(O)₂NHpentyl, or S(O)₂NHhexyl, or such as S(O)₂NHmethyl. In still other embodiments, R^{A} is substituted by —S(O)₀₋₂(C₁₋₆alkyl)₂ such as S(O)₂(C₁₋₆alkyl)₂, or such as S(O)₂(methyl)₂, S(O)₂(ethyl)₂, S(O)₂(propyl)₂, S(O)₂(butyl)₂, S(O)N(pentyl)₂, or S(O)₂(hexyl)₂, or such as S(O)₂(methyl)₂. In yet further embodiments, R^{A} is substituted by —S(O)₀₋₂(heteroaryl) such as —S-(heteroaryl), substituted by —S(O)(heteroaryl), or —S(O)₂(heteroaryl). In other embodiments, R^{A} is substituted by —S(O)₀₋₂(heterocyclyl) such as —S-(heterocyclyl), —S(O)(heterocyclyl), or —S(O)₂(heterocyclyl). In further embodiments, R^{A} is substituted by —C(O)(C₁₋₆alkyl) such as C(O)methyl, C(O)ethyl, C(O)propyl, C(O)butyl, C(O)pentyl, or C(O)hexyl, or such as C(O)methyl. In yet other embodiments, R^{A} is substituted by —C(O)NH₂. In still other embodiments, R^{A} is substituted by —C(O)NHC₁₋₆alkyl such as C(O)NHmethyl, C(O)NHethyl, C(O)NHpropyl, C(O)NHbutyl, C(O)NHpentyl, or C(O)NHhexyl, or such as C(O)NHmethyl. In yet further embodiments, R^{A} is substituted by —C(O)N(C₁₋₆alkyl)₂ such as C(O)N(methyl)₂, C(O)N(ethyl)₂, C(O)N(propyl)₂, C(O)N(butyl)₂, C(O)N(pentyl)₂, or C(O)N(hexyl)₂, or such as C(O)N(methyl)₂. In other embodiments, R^{A} is substituted by —C(O)(C₃₋₁₄carbocyclyl) such as —C(O)(cyclopropyl), C(O)(cyclobutyl), C(O)(cyclopentyl), C(O)(cyclohexyl), C(O)(cyclopropenyl), C(O)(cyclobutenyl), C(O)(cyclopentenyl), or C(O)(cyclohexenyl), or such as C(O)(cyclopropyl). In further embodiments, R^{A} is substituted by —C(O)(heteroaryl). In yet other embodiments, R^{A} is substituted by —C(O)(heterocyclyl). In still further embodiments, R^{A} is substituted by —C(O)(C₆₋₁₄aryl) such as —C(O)(phenyl). In other embodiments, R^{A} is substituted by —C₀₋₆alkyl-C₃₋₁₄carbocyclyl such as —C₃₋₁₄carbocyclyl or —CH₂—C₃₋₁₄carbocyclyl, or such as cyclopropyl, cyclobutyl, cyclopentyl, cyclohexyl, cyclopropenyl, cyclobutenyl, cyclopentenyl, or cyclohexenyl, or such as —CH₂-cyclopropyl, cyclobutyl, cyclopentyl, cyclohexyl, cyclopropenyl, cyclobutenyl, cyclopentenyl, or cyclohexenyl. In further embodiments, R^{A} is substituted by —C₀₋₆alkyl-C₆₋₁₄aryl such as —C₆₋₁₄aryl or CH₂—C₆₋₁₄aryl, or such as phenyl, or such as —CH₂-phenyl. In still other embodiments, R^{A} is substituted by —C₀₋₆alkyl-O—C₆₋₁₄aryl such as —O—C₆₋₁₄aryl or CH₂—O—C₆₋₁₄aryl, or such as —O-phenyl, or such as —CH₂—O-phenyl. In yet further embodiments, R^{A} is substituted by —C₀₋₆alkyl-(3- to 14-membered heterocyclyl) such as −3- to 14-membered heterocyclyl or CH₂-(3- to 14-membered heterocyclyl). In other embodiments, R^{A} is substituted by —C₀₋₆alkyl-(3- to 14-membered heteroaryl) such as -heteroaryl or CH₂-(heteroaryl). In further embodiments, R^{A} is substituted by —C₀₋₆alkyl-O-(3- to 14-membered heterocyclyl) such as —O-3- to 14-membered heterocyclyl or CH₂—O-(3- to 14-membered heterocyclyl). In still further embodiments, R^{A} is substituted by —C₀₋₆alkyl-NH(alkyl) such as —NH(alkyl) or —CH₂—NH(alkyl), or such as —NH(methyl), NH(ethyl), NH(propyl), NH(butyl), NH(pentyl), or NH(hexyl), or such as CH₂—NH(methyl), —CH₂—NH(ethyl), —CH₂—NH(propyl), —CH₂—NH(butyl), —CH₂—NH(pentyl), or —CH₂—NH(hexyl). In other embodiments, R^{A} is substituted by —C₀₋₆alkyl-N(alkyl)₂ such as —N(alkyl)₂ or —CH₂—N(alkyl)₂, or such as —N(methyl)₂, N(ethyl)₂, N(propyl)₂, N(butyl)₂, N(pentyl)₂, or N(hexyl)₂, or such as —CH₂—N(methyl)₂, —CH₂—N(ethyl)₂, —CH₂—N(propyl)₂, —CH₂—N(butyl)₂, —CH₂—N(pentyl)₂, or —CH₂—N(hexyl)₂.

R^{B} and R^{C} are, independently, H, D, CN, NH₂, halo, OH, C₁₋₆alkyl, deuterated C₁₋₆alkyl, C₁₋₆haloalkyl, —C₀₋₆alkyl-C₃₋₁₄carbocyclyl, C₁₋₆alkoxy, C₁₋₆haloalkoxy, deuterated C₁₋₆alkoxy, —C₁₋₆alkylOH, C₂₋₆alkenyl, C₂₋₆haloalkenyl, C₂₋₆alkynyl, —S(O)₀₋₂(C₁₋₆alkyl), —S(O)₀₋₂NH₂, —S(O)₀₋₂NHC₁₋₆alkyl, —S(O)₀₋₂(C₁₋₆alkyl)₂, —S(O)₀₋₂(C₆₋₁₄aryl), —S(O)₀₋₂(heteroaryl), —S(O)₀₋₂(heterocyclyl), —C(O)(C₁₋₆alkyl), —C(O)NH₂, —C(O)NHC₁₋₆alkyl, —C(O)NH(C₁₋₆alkyl)₂, —C(O)(C₃₋₁₄carbocyclyl), —C(O)(heteroaryl), —C(O)(heterocyclyl), —C(O)(C₆₋₁₄aryl), —C₀₋₆alkyl-C₆₋₁₄aryl, —C₀₋₆alkyl-O—C₆₋₁₄aryl, —C₀₋₆alkyl-(3- to 14-membered heterocyclyl), —C₀₋₆alkyl-(3- to 14-membered heteroaryl), —C₀₋₆alkyl-O-(3- to 14-membered heterocyclyl), —C₀₋₆alkyl-NH₂, —C₀₋₆alkyl-NH(alkyl), or —C₀₋₆alkyl-N(alkyl)₂. In some embodiments, R^{B} and/or R^{C} is H. In other embodiments, R^{B} and/or R^{C} is D. In other embodiments, R^{B} and/or R^{C} is CN. In further embodiments, R^{B} and/or R^{C} is NH₂. In yet other embodiments, R^{B} and/or R^{C} is halo. In still further embodiments, R^{B} and/or R^{C} is C₁₋₆alkyl such as methyl, ethyl, propyl, butyl, pentyl, or hexyl, or such as methyl, or such as ethyl, or such as propyl, or such as butyl. In other embodiments, R^{B} and/or R^{C} is deuterated C₁₋₆alkyl such as deuterated C₁₋₆alkyl, or such as CH₂D, CHD₂, or CD₃, or such as CD₃. In further embodiments, R^{B} and/or R^{C} is C₁₋₆haloalkyl such as C₁₋₆fluoroalkyl, or such as CF₃, CHF₂, CH₂F, CH₂CF₃, CH₂CF₂H, CH₂CH₂F, or CH₂CH₂CF₃, or such as CF₃, or such as CH₂CF₃, or such as CH₂CH₂CF₃. In yet other embodiments, R^{B} and/or R^{C} is C₁₋₆alkoxy such as methoxy, ethoxy, propoxy, butoxy, pentoxy, or hexoxy, or such as methoxy. In still further embodiments, R^{B} and/or R^{C} is C₁₋₆haloalkoxy such as C₁₋₆fluoroalkoxy, or such as OCF₃, OCHF₂, OCH₂F, or such as OCF₃. In other embodiments, R^{B} and/or R^{C} is deuterated C₁₋₆alkoxy such as OCH₂D, OCHD₂, or OCD₃, or such as OCD₃. In further embodiments, R^{B} and/or R^{C} is OH. In still other embodiments, R^{B} and/or R^{C} is —C₁₋₆alkylOH such as CH₂OH, CH₂CH₂OH, propyl-OH, butyl-OH, pentyl-OH, or hexyl-OH, or such as CH₂OH. In yet further embodiments, R^{B} and/or R^{C} is C₂₋₆alkenyl such as ethenyl, propenyl, butenyl, pentenyl, or hexenyl, or such as propenyl. In other embodiments, R^{B} and/or R^{C} is C₂₋₆haloalkenyl such as C₂₋₆fluoroalkenyl, or such as fluoroethenyl, fluoropropenyl, fluorobutenyl, fluoropentenyl, or fluorohexenyl, or such as fluoropropenyl. In further embodiments, R^{B} and/or R^{C} is C₂₋₆alkynyl such as ethynyl, propynyl, butynyl, pentynyl, or hexynyl, or such as propynyl. In yet other embodiments, R^{B} and/or R^{C} is —S(O)₀₋₂(C₁₋₆alkyl) such as S(O)₂(C₁₋₆alkyl), or such as S(O)₂(methyl), S(O)₂(ethyl), S(O)₂(propyl), S(O)₂(butyl), S(O)₂(pentyl), or S(O)₂(hexyl), or such as S(O)₂(methyl). In still further embodiments, R^{B} and/or R^{C} is —S(O)₀₋₂(C₆₋₁₄aryl) such as —S—(C₆₋₁₄aryl), —S(O)(C₆₋₁₄aryl), or —S(O)₂(C₆₋₁₄aryl), or such as —S-(phenyl), or such as —S(O)(phenyl), or such as —S(O)₂(phenyl). In other embodiments, R^{B} and/or R^{C} is —S(O)₀₋₂NH₂ such as —S—NH₂, S(O)NH₂, or S(O)₂NH₂, or such as S(O)₂NH₂. In further embodiments, R^{B} and/or R^{C} is —S(O)₀₋₂NHC₁₋₆alkyl such as S(O)₂NHC₁₋₆alkyl, or such as S(O)₂NHmethyl, S(O)₂NHethyl, S(O)₂NHpropyl, S(O)₂NHbutyl, S(O)₂NHpentyl, or S(O)₂NHhexyl, or such as S(O)₂NHmethyl. In still other embodiments, R^{B} and/or R^{C} is —S(O)₀₋₂(C₁₋₆alkyl)₂ such as S(O)₂(C₁₋₆alkyl)₂, or such as S(O)₂(methyl)₂, S(O)₂(ethyl)₂, S(O)₂(propyl)₂, S(O)₂(butyl)₂, S(O)₂(pentyl)₂, or S(O)₂(hexyl)₂, or such as S(O)₂(methyl)₂. In yet further embodiments, R^{B} and/or R^{C} is —S(O)₀₋₂(heteroaryl) such as —S-(heteroaryl), —S(O)(heteroaryl), or —S(O)₂(heteroaryl). In other embodiments, R^{B} and/or R^{C} is —S(O)₀₋₂(heterocyclyl) such as —S-(heterocyclyl), —S(O)(heterocyclyl), or —S(O)₂(heterocyclyl). In further embodiments, R^{B} and/or R^{C} is —C(O)(C₁₋₆alkyl) such as C(O)methyl, C(O)ethyl, C(O)propyl, C(O)butyl, C(O)pentyl, or C(O)hexyl, or such as C(O)methyl. In yet other embodiments, R^{B} and/or R^{C} is —C(O)NH₂. In still other embodiments, R^{B} and/or R^{C} is —C(O)NHC₁₋₆alkyl such as C(O)NHmethyl, C(O)NHethyl, C(O)NHpropyl, C(O)NHbutyl, C(O)NHpentyl, or C(O)NHhexyl, or such as C(O)NHmethyl. In yet further embodiments, R^{B} and/or R^{C} is —C(O)N(C₁₋₆alkyl)₂ such as C(O)N(methyl)₂, C(O)N(ethyl)₂, C(O)N(propyl)₂, C(O)N(butyl)₂, C(O)N(pentyl)₂, or C(O)N(hexyl)₂, or such as C(O)N(methyl)₂. In other embodiments, R^{B} and/or R^{C} is —C(O)(C₃₋₁₄carbocyclyl) such as —C(O)(cyclopropyl), C(O)(cyclobutyl), C(O)(cyclopentyl), C(O)(cyclohexyl), C(O)(cyclopropenyl), C(O)(cyclobutenyl), C(O)(cyclopentenyl), or C(O)(cyclohexenyl), or such as C(O)(cyclopropyl). In further embodiments, R^{B} and/or R^{C} is —C(O)(heteroaryl). In yet other embodiments, R^{B} and/or R^{C} is —C(O)(heterocyclyl). In still further embodiments, R^{B} and/or R^{C} is —C(O)(C₆₋₁₄aryl) such as —C(O)(phenyl). In other embodiments, R^{B} and/or R^{C} is —C₀₋₆alkyl-C₃₋₁₄carbocyclyl such as —C₃₋₁₄carbocyclyl or —CH₂—C₃₋₁₄carbocyclyl, or such as cyclopropyl, cyclobutyl, cyclopentyl, cyclohexyl, cyclopropenyl, cyclobutenyl, cyclopentenyl, or cyclohexenyl, or such as —CH₂-cyclopropyl, —CH₂-cyclobutyl, —CH₂-cyclopentyl, —CH₂-cyclohexyl, —CH₂-cyclopropenyl, —CH₂-cyclobutenyl, —CH₂-cyclopentenyl, or —CH₂-cyclohexenyl. In further embodiments, R^{B} and/or R^{C} is —C₀₋₆alkyl-C₆₋₁₄aryl such as —C₆₋₁₄aryl or CH₂—C₆₋₁₄aryl, or such as phenyl, or such as —CH₂-phenyl. In still other embodiments, R^{B} and/or R^{C} is —C₀₋₆alkyl-O—C₆₋₁₄aryl such as —O—C₆₋₁₄aryl or CH₂—O—C₆₋₁₄aryl, or such as —O-phenyl, or such as —CH₂—O-phenyl. In yet further embodiments, R^{B} and/or R^{C} is —C₀₋₆alkyl-(3- to 14-membered heterocyclyl) such as −3- to 14-membered heterocyclyl or CH₂-(3- to 14-membered heterocyclyl). In other embodiments, R^{B} and/or R^{C} is —C₀₋₆alkyl-(3- to 14-membered heteroaryl) such as -heteroaryl or CH₂-(heteroaryl). In further embodiments, R^{B} and/or R^{C} is —C₀₋₆alkyl-O-(3- to 14-membered heterocyclyl) such as —O-3- to 14-membered heterocyclyl or CH₂—O-(3- to 14-membered heterocyclyl). In yet other embodiments, R^{B} and/or R^{C} —C₀₋₆alkyl-NH₂ such as —NH₂, CH₂NH₂, CH₂CH₂NH₂, or -pentyl-NH₂. In still further embodiments, R^{B} and/or R^{C} —C₀₋₆alkyl-NH(alkyl) such as —NH(alkyl) or —CH₂—NH(alkyl), or such as —NH(methyl), NH(ethyl), NH(propyl), NH(butyl), NH(pentyl), or NH(hexyl), or such as CH₂—NH(methyl), —CH₂—NH(ethyl), —CH₂—NH(propyl), —CH₂—NH(butyl), —CH₂—NH(pentyl), or —CH₂—NH(hexyl). In other embodiments, R^{B} and/or R^{C} —C₀₋₆alkyl-N(alkyl)₂ such as —N(alkyl)₂ or —CH₂—N(alkyl)₂, or such as —N(methyl)₂, N(ethyl)₂, N(propyl)₂, N(butyl)₂, N(pentyl)₂, or N(hexyl)₂, or such as —CH₂—N(methyl)₂, —CH₂—N(ethyl)₂, —CH₂—N(propyl)₂, —CH₂—N(butyl)₂, —CH₂—N(pentyl)₂, or —CH₂—N(hexyl)₂.

R^{A} and/or R^{B} is optionally substituted, as permitted, by D, CN, halo, OH, C₁₋₆alkyl, deuterated C₁₋₆alkyl, C₁₋₆haloalkyl, —C₀₋₆alkyl-C₃₋₁₄carbocyclyl, C₁₋₆alkoxy, C₁₋₆haloalkoxy, deuterated C₁₋₆alkoxy, —C₁₋₆alkylOH, C₂₋₆alkenyl, C₂₋₆haloalkenyl, C₂₋₆alkynyl, —S(O)₀₋₂(C₁₋₆alkyl), —S(O)₀₋₂(C₆₋₁₄aryl), —S(O)₀₋₂(heteroaryl), —S(O)₀₋₂(heterocyclyl), —S(O)₀₋₂NH₂, —S(O)₀₋₂NHC₁₋₆alkyl, —S(O)₀₋₂(C₁₋₆alkyl)₂, —C(O)(C₁₋₆alkyl), —C(O)NH₂, —C(O)NHC₁₋₆alkyl, —C(O)NH(C₁₋₆alkyl)₂, —C(O)(C₃₋₁₄carbocyclyl), —C(O)(C₆₋₁₄aryl), —C(O)(heteroaryl), —C(O)(heterocyclyl), —C₀₋₆alkyl-O—C₆₋₁₄aryl, —C₀₋₆alkyl-C₆₋₁₄aryl, —C₀₋₆alkyl-(3- to 14-membered heterocyclyl), —C₀₋₆alkyl-3- to 14-membered heteroaryl, or —C₀₋₆alkyl-O-(3- to 14-membered heterocyclyl), —C₀₋₆alkyl-NH₂, —C₀₋₆alkyl-NH(alkyl), or —C₀₋₆alkyl-N(alkyl)₂. In some embodiments, R^{B} and/or R^{C} is substituted by D. In other embodiments, R^{B} and/or R^{C} is substituted by CN. In yet other embodiments, R^{B} and/or R^{C} is substituted by halo. In still further embodiments, R^{B} and/or R^{C} is substituted by C₁₋₆alkyl such as methyl, ethyl, propyl, butyl, pentyl, or hexyl, or such as methyl, or such as ethyl, or such as propyl, or such as butyl. In other embodiments, R^{B} and/or R^{C} is substituted by deuterated C₁₋₆alkyl such as CH₂D, CHD₂, or CD₃, or such as CD₃. In further embodiments, R^{B} and/or R^{C} is substituted by C₁₋₆haloalkyl such as C₁₋₆fluoroalkyl, or such as CF₃, CHF₂, CH₂F, CH₂CF₃, CH₂CF₂H, CH₂CH₂F, or CH₂CH₂CF₃, or such as CF₃, or such as CH₂CF₃, or such as CH₂CH₂CF₃. In yet other embodiments, R^{B} and/or R^{C} is substituted by C₁₋₆alkoxy such as methoxy, ethoxy, propoxy, butoxy, pentoxy, or hexoxy, or such as methoxy. In still further embodiments, R^{B} and/or R^{C} is substituted by C₁₋₆haloalkoxy such as C₁₋₆fluoroalkoxy, or such as OCF₃, OCHF₂, OCH₂F, or such as OCF₃. In other embodiments, R^{B} and/or R^{C} is substituted by deuterated C₁₋₆alkoxy such as OCH₂D, OCHD₂, or OCD₃, or such as OCD₃. In further embodiments, R^{B} and/or R^{C} is substituted by OH. In still other embodiments, R^{B} and/or R^{C} is substituted by —C₁₋₆alkylOH such as CH₂OH, CH₂CH₂OH, propyl-OH, butyl-OH, pentyl-OH, or hexyl-OH, or such as CH₂OH. In yet further embodiments, R^{B} and/or R^{C} is substituted by C₂₋₆alkenyl such as ethenyl, propenyl, butenyl, pentenyl, or hexenyl, or such as propenyl. In other embodiments, R^{B} and/or R^{C} is substituted by C₂₋₆haloalkenyl such as C₂₋₆fluoroalkenyl, or such as fluoroethenyl, fluoropropenyl, fluorobutenyl, fluoropentenyl, or fluorohexenyl, or such as fluoropropenyl. In further embodiments, R^{B} and/or R^{C} is substituted by C₂₋₆alkynyl such as ethynyl, propynyl, butynyl, pentynyl, or hexynyl, or such as propynyl. In yet other embodiments, R^{B} and/or R^{C} is substituted by —S(O)₀₋₂(C₁₋₆alkyl) such as S(O)₂(C₁₋₆alkyl), or such as S(O)₂(methyl), S(O)₂(ethyl), S(O)₂(propyl), S(O)₂(butyl), S(O)₂(pentyl), or S(O)₂(hexyl), or such as S(O)₂(methyl). In still further embodiments, R^{B} and/or R^{C} is substituted by —S(O)₀₋₂(C₆₋₁₄aryl) such as —S—(C₆₋₁₄aryl), —S(O)(C₆₋₁₄aryl), or —S(O)₂(C₆₋₁₄aryl), or such as —S-(phenyl), —S(O)(phenyl), or —S(O)₂(phenyl). In other embodiments, R^{B} and/or R^{C} is substituted by —S(O)₀₋₂NH₂, or such as —S—NH₂, S(O)NH₂, or S(O)₂NH₂, or such as S(O)₂NH₂. In further embodiments, R^{B} and/or R^{C} is substituted by —S(O)₀₋₂NHC₁₋₆alkyl such as S(O)₂NHC₁₋₆alkyl, or such as S(O)₂NHmethyl, S(O)₂NHethyl, S(O)₂NHpropyl, S(O)₂NHbutyl, S(O)₂NHpentyl, or S(O)₂NHhexyl, or such as S(O)₂NHmethyl. In still other embodiments, R^{B} and/or R^{C} is substituted by —S(O)₀₋₂(C₁₋₆alkyl)₂ such as S(O)₂(C₁₋₆alkyl)₂, or such as S(O)₂(methyl)₂, S(O)₂(ethyl)₂, S(O)₂(propyl)₂, S(O)₂(butyl)₂, S(O)N(pentyl)₂, or S(O)₂(hexyl)₂, or such as S(O)₂(methyl)₂. In yet further embodiments, R^{B} and/or R^{C} is substituted by —S(O)₀₋₂(heteroaryl) such as —S-(heteroaryl), or such as —S(O)(heteroaryl) or —S(O)₂(heteroaryl). In other embodiments, R^{B} and/or R^{C} is substituted by —S(O)₀₋₂(heterocyclyl) such as —S-(heterocyclyl), —S(O)(heterocyclyl), or —S(O)₂(heterocyclyl). In further embodiments, R^{B} and/or R^{C} is substituted by —C(O)(C₁₋₆alkyl) such as C(O)methyl, C(O)ethyl, C(O)propyl, C(O)butyl, C(O)pentyl, or C(O)hexyl, or such as C(O)methyl. In yet other embodiments, R^{B} and/or R^{C} is substituted by —C(O)NH₂. In still other embodiments, R^{B} and/or R^{C} is substituted by —C(O)NHC₁₋₆alkyl such as C(O)NHmethyl, C(O)NHethyl, C(O)NHpropyl, C(O)NHbutyl, C(O)NHpentyl, or C(O)NHhexyl, or such as C(O)NHmethyl.

In yet further embodiments, R^{B} and/or R^{C} is substituted by —C(O)N(C₁₋₆alkyl)₂ such as C(O)N(methyl)₂, C(O)N(ethyl)₂, C(O)N(propyl)₂, C(O)N(butyl)₂, C(O)N(pentyl)₂, or C(O)N(hexyl)₂, or such as C(O)N(methyl)₂. In other embodiments, R^{B} and/or R^{C} is substituted by —C(O)(C₃₋₁₄carbocyclyl) such as —C(O)(cyclopropyl), C(O)(cyclobutyl), C(O)(cyclopentyl), C(O)(cyclohexyl), C(O)(cyclopropenyl), C(O)(cyclobutenyl), C(O)(cyclopentenyl), or C(O)(cyclohexenyl), or such as C(O)(cyclopropyl). In further embodiments, R^{B} and/or R^{C} is substituted by —C(O)(heteroaryl). In yet other embodiments, R^{B} and/or R^{C} is substituted by —C(O)(heterocyclyl). In still further embodiments, R^{B} and/or R^{C} is substituted by —C(O)(C₆₋₁₄aryl) such as —C(O)(phenyl). In other embodiments, R^{B} and/or R^{C} is substituted by —C₀₋₆alkyl-C₃₋₁₄carbocyclyl such as —C₃₋₁₄carbocyclyl or —CH₂—C₃₋₁₄carbocyclyl, or such as cyclopropyl, cyclobutyl, cyclopentyl, cyclohexyl, cyclopropenyl, cyclobutenyl, cyclopentenyl, or cyclohexenyl, or such as —CH₂-cyclopropyl, cyclobutyl, cyclopentyl, cyclohexyl, cyclopropenyl, cyclobutenyl, cyclopentenyl, or cyclohexenyl. In further embodiments, R^{B} and/or R^{C} is substituted by —C₀₋₆alkyl-C₆₋₁₄aryl such as —C₆₋₁₄aryl or CH₂—C₆₋₁₄aryl, or such as phenyl, or such as —CH₂-phenyl. In still other embodiments, R^{B} and/or R^{C} is substituted by —C₀₋₆alkyl-O—C₆₋₁₄aryl such as —O—C₆₋₁₄aryl or CH₂—O—C₆₋₁₄aryl, or such as —O-phenyl, or such as —CH₂—O-phenyl. In yet further embodiments, R^{B} and/or R^{C} is substituted by —C₀₋₆alkyl-(3- to 14-membered heterocyclyl) such as −3- to 14-membered heterocyclyl or CH₂-(3- to 14-membered heterocyclyl). In other embodiments, R^{B} and/or R^{C} is substituted by —C₀₋₆alkyl-(3- to 14-membered heteroaryl) such as -heteroaryl or CH₂-(heteroaryl). In further embodiments, R^{B} and/or R^{C} is substituted by —C₀₋₆alkyl-O-(3- to 14-membered heterocyclyl) such as —O-3- to 14-membered heterocyclyl or CH₂—O-(3- to 14-membered heterocyclyl). In still further embodiments, R^{B} and/or R^{C} is substituted by —C₀₋₆alkyl-NH(alkyl) such as —NH(alkyl) or —CH₂—NH(alkyl), or such as —NH(methyl), NH(ethyl), NH(propyl), NH(butyl), NH(pentyl), or NH(hexyl), or such as CH₂—NH(methyl), —CH₂—NH(ethyl), —CH₂—NH(propyl), —CH₂—NH(butyl), —CH₂—NH(pentyl), or —CH₂—NH(hexyl). In other embodiments, R^{B} and/or R^{C} is substituted by —C₀₋₆alkyl-N(alkyl)₂ such as —N(alkyl)₂ or —CH₂—N(alkyl)₂, such as —N(methyl)₂, N(ethyl)₂, N(propyl)₂, N(butyl)₂, N(pentyl)₂, or N(hexyl)₂, or such as —CH₂—N(methyl)₂, —CH₂—N(ethyl)₂, —CH₂—N(propyl)₂, —CH₂—N(butyl)₂, —CH₂—N(pentyl)₂, or —CH₂—N(hexyl)₂.

X¹ is N, NR⁶, CR⁴, or CR⁴R^{4′}. In some embodiments, X¹ is N. In other embodiments, X¹ is NR⁶. In further embodiments, X¹ is CR⁴. In yet other embodiments, X¹ is CR⁴R^{4′}. R⁶ is H or C₁₋₆alkyl. In some embodiments, R⁶ is H. In other embodiments, R⁶ is C₁₋₆alkyl, such as methyl, ethyl, propyl, butyl, pentyl, or hexyl, or such as methyl.

X² is N, CR⁵, or CR⁵R⁵. In some embodiments, X² is N. In other embodiments, X² is CR⁵. In further embodiments, X² is CR⁵R^{5′}. R⁵ and R^{5′} are, independently, defined above. Alternatively, R⁵ and R^{5′} are combined to form oxo (═O).

X³ is N, NR³, or CR³. In some embodiments, X³ is N. In other embodiments, X³ is NR³ or CR³. In further embodiments, X³ is NR³. In yet other embodiments, X³ is CR³.

X¹ [symbol] X² and X² [symbol] X³ are, independently, a double or single bond, provided that (i) both X¹ [symbol] X² and X² [symbol] X³ are not double bonds and (i) both X¹ [symbol] X² and X² [symbol] X³ are single bonds when X² is C(O). In some embodiments, X¹ [symbol] X² is a single bond. In other embodiments, X¹ [symbol] X² is a double bond. In further embodiments, X² [symbol] X³ is a single bond. In yet other embodiments, X² [symbol] X³ is a double bond. In still further embodiments, X¹ [symbol] X² and X² [symbol] X³ are single bonds. In other embodiments, X¹ [symbol] X² is a single bond and X² [symbol] X³ is a double bond. In further embodiments, X¹ [symbol] X² is a double bond and X² [symbol] X³ is a double bond.

In some aspects, the compound of the application is of Formula II:

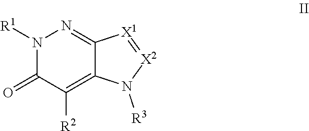

or a pharmaceutically acceptable salt thereof.

In other aspects, the compound of the application is of Formula III:

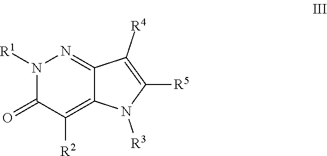

or a pharmaceutically acceptable salt thereof.

In further aspects, the compound of the application is of Formula IV:

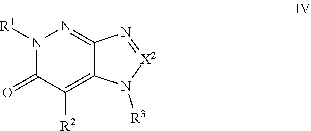

or a pharmaceutically acceptable salt thereof.

In yet other aspects, the compound of the application is of Formula V:

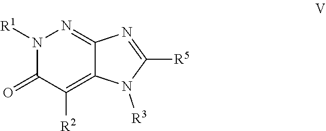

or a pharmaceutically acceptable salt thereof.

In still further aspects, the compound of the application is Formula VI:

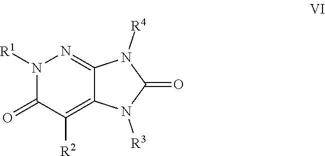

or a pharmaceutically acceptable salt thereof.

In other aspects, the compound of the application is of Formula VII:

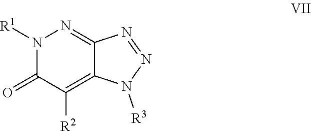

or a pharmaceutically acceptable salt thereof.

In further aspects, the compound of the application is of Formula VIII:

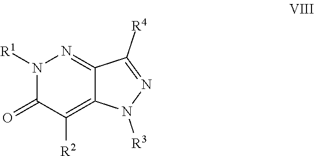

or a pharmaceutically acceptable salt thereof.

In still other aspects, the compound of the application is of Formula IX:

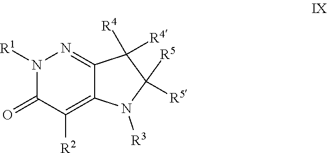

or a pharmaceutically acceptable salt thereof.

In yet further aspects, the compound of the application is of Formula X:

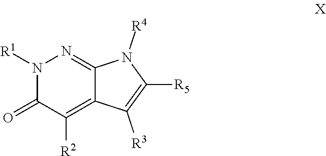

or a pharmaceutically acceptable salt thereof.

In other aspects, the compound of the application is of Formula XIa, XIb, or XIc:

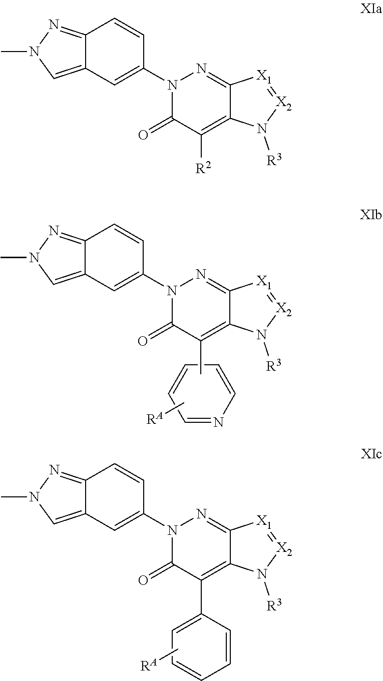

or a pharmaceutically acceptable salt thereof. In some embodiments, the compound of the application is of Formula Xia or a pharmaceutically acceptable salt thereof. In other embodiments, the compound of the application is of Formula XIb or a pharmaceutically acceptable salt thereof. In further embodiments, the compound of the application is of Formula XIc or a pharmaceutically acceptable salt thereof. In these compounds, R⁷ is H, C₁₋₆alkyl, deuterated C₁₋₆alkyl, —C₁₋₆alkyl-C₃₋₈carbocyclyl, C₁₋₆alkoxy, or deuterated C₁₋₆alkoxy. In some embodiments, R⁷ is H. In other embodiments, R⁷ is C₁₋₆alkyl, such as methyl, ethyl, propyl, butyl, pentyl, or hexyl, or such as methyl. In further embodiments, R⁷ is deuterated C₁₋₆alkyl, such as CH₂D, CHD₂, CD₃, CH₂CH₂D, CH₂CHD₂, or CH₂CD₃, or such as CD₃. In yet other embodiments, R⁷ is —C₁₋₆alkyl-C₃₋₈carbocyclyl, such as —CH₂—C₃₋₈carbocyclyl, or such as —C₁₋₆alkyl-cyclopropyl, or such as —CH₂-cyclopropyl. In yet further embodiments, R⁷ is C₁₋₆alkoxy, such as methoxy, ethoxy, propoxy, butoxy, pentoxy, or hexoxy. In other embodiments, R⁷ is deuterated C₁₋₆alkoxy such as OCH₂D, OCHD₂, OCD₃, OCH₂CH₂D, OCH₂CHD₂, or OCH₂CD₃.

In further aspects, the compound of the application is of Formula XIa, XIb, or XIc:

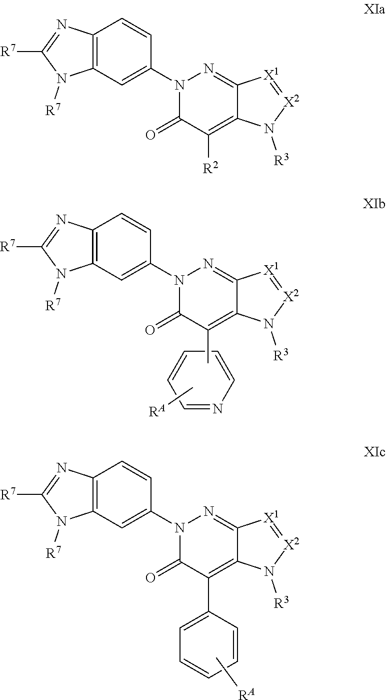

or a pharmaceutically acceptable salt thereof. In some embodiments, the compound of the application is of Formula XIIa or a pharmaceutically acceptable salt thereof. In other embodiments, the compound of the application is of Formula XIIb or a pharmaceutically acceptable salt thereof. In further embodiments, the compound of the application is of Formula XIIc or a pharmaceutically acceptable salt thereof. In these compounds, R⁷ is H, C₁₋₆alkyl, deuterated C₁₋₆alkyl, —C₁₋₆alkyl-C₃₋₈carbocyclyl, C₁₋₆alkoxy, or deuterated C₁₋₆alkoxy. In some embodiments, R⁷ is H. In other embodiments, R⁷ is C₁₋₆alkyl, such as methyl, ethyl, propyl, butyl, pentyl, or hexyl, or such as methyl. In further embodiments, R⁷ is deuterated C₁₋₆alkyl, such as CH₂D, CHD₂, CD₃, CH₂CH₂D, CH₂CHD₂, or CH₂CD₃, or such as CD₃. In yet other embodiments, R⁷ is —C₁₋₆alkyl-C₃₋₈carbocyclyl, such as —CH₂—C₃₋₈carbocyclyl, or such as —C₁₋₆alkyl-cyclopropyl, or such as —CH₂-cyclopropyl. In yet further embodiments, R⁷ is C₁₋₆alkoxy, such as methoxy, ethoxy, propoxy, butoxy, pentoxy, or hexoxy. In other embodiments, R⁷ is deuterated C₁₋₆alkoxy such as OCH₂D, OCHD₂, OCD₃, OCH₂CH₂D, OCH₂CHD₂, or OCH₂CD₃.

In yet other aspects, the compound of the application is of Formula XIIIa, XIIIb, or XIIIc:

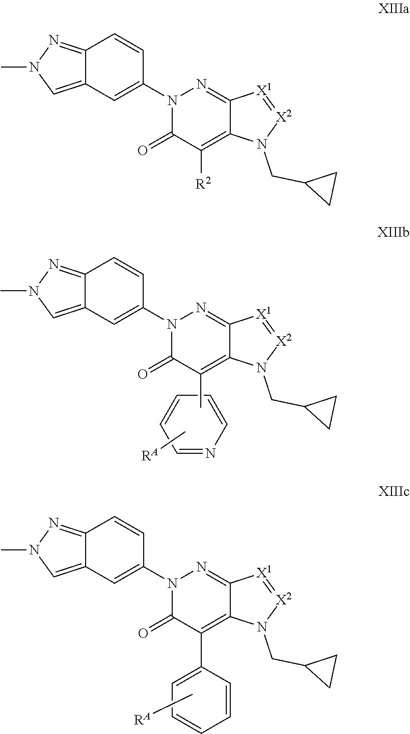

or a pharmaceutically acceptable salt thereof. In some embodiments, the compound of the application is of Formula XIIIa or a pharmaceutically acceptable salt thereof. In other embodiments, the compound of the application is of Formula XIIIb or a pharmaceutically acceptable salt thereof. In further embodiments, the compound of the application is of Formula XIIIc or a pharmaceutically acceptable salt thereof.

In still further aspects, the compound of the application is:
Example 101: 5-(cyclopropylmethyl)-4-(4-(difluoromethoxy)phenyl)-2-(2-methyl-2H-indazol-5-yl)-2,5-dihydro-3H-pyrrolo[3,2-c]pyridazin-3-one;
Example 102: 5-(cyclopropylmethyl)-4-(4-(difluoromethoxy)phenyl)-2-(2-methyl-2H-indazol-5-yl)-3-oxo-3,5-dihydro-2H-pyrrolo[3,2-c]pyridazine-7-carbonitrile;
Example 103: 5-(cyclopropylmethyl)-2-(2-methyl-2H-indazol-5-yl)-4-(6-methylpyridin-3-yl)-2,5-dihydro-3H-pyrrolo[3,2-c]pyridazin-3-one;
Example 104: 5-(cyclopropylmethyl)-2-(2-methyl-2H-indazol-5-yl)-4-(6-methylpyridin-3-yl)-3-oxo-3,5-dihydro-2H-pyrrolo[3,2-c]pyridazine-7-carbonitrile;
Example 105: 4-(4-(difluoromethoxy)phenyl)-2-(2-methyl-2H-indazol-5-yl)-5-(3,3,3-trifluoropropyl)-2,5-dihydro-3H-pyrrolo[3,2-c]pyridazin-3-one;
Example 106: 5-(cyclopropylmethyl)-4-(6-cyclopropylpyridin-3-yl)-2-(2-methyl-2H-indazol-5-yl)-2,5-dihydro-3H-pyrrolo[3,2-c]pyridazin-3-one;
Example 107: 5-(cyclopropylmethyl)-4-(6-cyclopropylpyridin-3-yl)-2-(2-methyl-2H-indazol-5-yl)-3-oxo-3,5-dihydro-2H-pyrrolo[3,2-c]pyridazine-7-carbonitrile;
Example 108: 2-(2-methyl-2H-indazol-5-yl)-4-(6-methylpyridin-3-yl)-3-oxo-5-(3,3,3-trifluoropropyl)-3,5-dihydro-2H-pyrrolo[3,2-c]pyridazine-7-carbonitrile;
Example 109: 4-(6-cyclopropylpyridin-3-yl)-2-(2-methyl-2H-indazol-5-yl)-3-oxo-5-(3,3,3-trifluoropropyl)-3,5-dihydro-2H-pyrrolo[3,2-c]pyridazine-7-carbonitrile;
Example 110: 5-(cyclopropylmethyl)-4-(6-methoxypyridin-3-yl)-2-(2-methyl-2H-indazol-5-yl)-2,5-dihydro-3H-pyrrolo[3,2-c]pyridazin-3-one;
Example 111: 5-(cyclopropylmethyl)-4-(6-methoxypyridin-3-yl)-2-(2-methyl-2H-indazol-5-yl)-3-oxo-3,5-dihydro-2H-pyrrolo[3,2-c]pyridazine-7-carbonitrile;
Example 112: 4-(4-chlorophenyl)-5-(cyclopropylmethyl)-2-(2-methyl-2H-indazol-5-yl)-2,5-dihydro-3H-pyrrolo[3,2-c]pyridazin-3-one;
Example 113: 5-(cyclopropylmethyl)-4-(6-(difluoromethoxy)pyridin-3-yl)-2-(2-methyl-2H-indazol-5-yl)-2,5-dihydro-3H-pyrrolo[3,2-c]pyridazin-3-one;
Example 114: 5-(cyclopropylmethyl)-4-(6-(difluoromethoxy)pyridin-3-yl)-2-(2-methyl-2H-indazol-5-yl)-3-oxo-3,5-dihydro-2H-pyrrolo[3,2-c]pyridazine-7-carbonitrile;
Example 115: 4-(4-(difluoromethoxy)phenyl)-5-isobutyl-2-(2-methyl-2H-indazol-5-yl)-2,5-dihydro-3H-pyrrolo[3,2-c]pyridazin-3-one;
Example 116: 4-(4-(difluoromethoxy)phenyl)-5-isobutyl-2-(2-methyl-2H-indazol-5-yl)-3-oxo-3,5-dihydro-2H-pyrrolo[3,2-c]pyridazine-7-carbonitrile;
Example 117: 5-(cyclopropylmethyl)-4-(6-cyclopropylpyridin-3-yl)-7-iodo-2-(2-methyl-2H-indazol-5-yl)-2,5-dihydro-3H-pyrrolo[3,2-c]pyridazin-3-one;
Example 118: 5-(cyclopropylmethyl)-2-(2-methyl-2H-indazol-5-yl)-4-(6-(trifluoromethyl)pyridin-3-yl)-2,5-dihydro-3H-pyrrolo[3,2-c]pyridazin-3-one;
Example 119: 5-(cyclopropylmethyl)-2-(2-methyl-2H-indazol-5-yl)-3-oxo-4-(6-(trifluoromethyl)pyridin-3-yl)-3,5-dihydro-2H-pyrrolo[3,2-c]pyridazine-7-carbonitrile;
Example 120: 4-(6-(1-cyanocyclopropyl)pyridin-3-yl)-5-(cyclopropylmethyl)-2-(2-methyl-2H-indazol-5-yl)-3-oxo-3,5-dihydro-2H-pyrrolo[3,2-c]pyridazine-7-carbonitrile;
Example 121: 4-(6-cyclopropylpyridin-3-yl)-5-isobutyl-2-(2-methyl-2H-indazol-5-yl)-3-oxo-3,5-dihydro-2H-pyrrolo[3,2-c]pyridazine-7-carbonitrile;
Example 122: 5-(cyclopropylmethyl)-4-(6-cyclopropylpyridin-3-yl)-2-(4-(methoxy-d3)phenyl)-2,5-dihydro-3H-pyrrolo[3,2-c]pyridazin-3-one;
Example 123: 5-(cyclopropylmethyl)-4-(6-(difluoromethyl)pyridin-3-yl)-2-(2-methyl-2H-indazol-5-yl)-2,5-dihydro-3H-pyrrolo[3,2-c]pyridazin-3-one;
Example 124: 5-(cyclopropylmethyl)-4-(6-cyclopropylpyridin-3-yl)-2-(4-(methoxy-d3)phenyl)-3-oxo-3,5-dihydro-2H-pyrrolo[3,2-c]pyridazine-7-carbonitrile;
Example 125: 5-(cyclopropylmethyl)-2-(2-methyl-2H-indazol-5-yl)-4-(5-(trifluoromethyl)pyridin-3-yl)-2,5-dihydro-3H-pyrrolo[3,2-c]pyridazin-3-one;
Example 126: 4-(5-chloropyridin-3-yl)-5-(cyclopropylmethyl)-2-(2-methyl-2H-indazol-5-yl)-2,5-dihydro-3H-pyrrolo[3,2-c]pyridazin-3-one;
Example 127: 4-(6-cyclopropylpyridin-3-yl)-2-(2-methyl-2H-indazol-5-yl)-3-oxo-5-propyl-3,5-dihydro-2H-pyrrolo[3,2-c]pyridazine-7-carbonitrile;
Example 128: 5-(cyclopropylmethyl)-4-(6-isopropylpyridin-3-yl)-2-(2-methyl-2H-indazol-5-yl)-3-oxo-3,5-dihydro-2H-pyrrolo[3,2-c]pyridazine-7-carbonitrile;
Example 129: 5-(cyclopropylmethyl)-4-(6-isopropylpyridin-3-yl)-2-(2-methyl-2H-indazol-5-yl)-2,5-dihydro-3H-pyrrolo[3,2-c]pyridazin-3-one;
Example 130: 4-(3-cyano-1H-indol-5-yl)-5-(cyclopropylmethyl)-2-(2-methyl-2H-indazol-5-yl)-3-oxo-3,5-dihydro-2H-pyrrolo[3,2-c]pyridazine-7-carbonitrile;
Example 131: 5-(cyclopropylmethyl)-2-(4-(methoxy-d3)phenyl)-4-(6-methylpyridin-3-yl)-3-oxo-3,5-dihydro-2H-pyrrolo[3,2-c]pyridazine-7-carbonitrile;
Example 132: 5-(cyclopropylmethyl)-4-(6-(difluoromethyl)pyridin-3-yl)-2-(4-(methoxy-d3)phenyl)-2,5-dihydro-3H-pyrrolo[3,2-c]pyridazin-3-one;
Example 133: 4-(6-cyclopropylpyridin-3-yl)-2-(2-methyl-2H-indazol-5-yl)-3-oxo-5-(2,2,2-trifluoroethyl)-3,5-dihydro-2H-pyrrolo[3,2-c]pyridazine-7-carbonitrile;
Example 134: 5-(cyclopropylmethyl)-2-(4-(methoxy-d3)phenyl)-4-(6-methylpyridin-3-yl)-2,5-dihydro-3H-pyrrolo[3,2-c]pyridazin-3-one;
Example 135: 5-(cyclopropylmethyl)-4-(6-ethylpyridin-3-yl)-2-(2-methyl-2H-indazol-5-yl)-2,5-dihydro-3H-pyrrolo[3,2-c]pyridazin-3-one;
Example 136: 5-(cyclopropylmethyl)-4-(6-ethylpyridin-3-yl)-2-(2-methyl-2H-indazol-5-yl)-3-oxo-3,5-dihydro-2H-pyrrolo[3,2-c]pyridazine-7-carbonitrile;
Example 137: 5-(cyclopropylmethyl)-4-(6-(difluoromethyl)pyridin-3-yl)-2-(4-(methoxy-d3)phenyl)-3-oxo-3,5-dihydro-2H-pyrrolo[3,2-c]pyridazine-7-carbonitrile;
Example 138: 5-(cyclopropylmethyl)-4-(4-cyclopropylphenyl)-2-(2-methyl-2H-indazol-5-yl)-3-oxo-3,5-dihydro-2H-pyrrolo[3,2-c]pyridazine-7-carbonitrile;
Example 139: 4-(4-chlorophenyl)-5-(cyclopropylmethyl)-2-(2-methyl-2H-indazol-5-yl)-3-oxo-3,5-dihydro-2H-pyrrolo[3,2-c]pyridazine-7-carbonitrile;
Example 140: 5-(cyclopropylmethyl)-4-(6-(difluoromethyl)pyridin-3-yl)-2-(1,2-dimethyl-1H-benzo[d]imidazol-6-yl)-2,5-dihydro-3H-pyrrolo[3,2-c]pyridazin-3-one;
Example 141: 5-(cyclopropylmethyl)-2-(1,2-dimethyl-1H-benzo[d]imidazol-6-yl)-4-(6-methylpyridin-3-yl)-2,5-dihydro-3H-pyrrolo[3,2-c]pyridazin-3-one;
Example 142: 5-(cyclopropylmethyl)-2-(1,2-dimethyl-1H-benzo[d]imidazol-6-yl)-4-(6-ethylpyridin-3-yl)-2,5-dihydro-3H-pyrrolo[3,2-c]pyridazin-3-one;
Example 143: 5-(cyclopropylmethyl)-4-(6-(difluoromethyl)pyridin-3-yl)-2-(2-methyl-2H-indazol-5-yl)-3-oxo-3,5-dihydro-2H-pyrrolo[3,2-c]pyridazine-7-carbonitrile;
Example 144: 5-(5-(cyclopropylmethyl)-2-(2-methyl-2H-indazol-5-yl)-3-oxo-3,5-dihydro-2H-pyrrolo[3,2-c]pyridazin-4-yl)-N-methylpicolinamide;
Example 145: 2-(2-methyl-2H-indazol-5-yl)-4-(6-methylpyridin-3-yl)-3-oxo-5-propyl-3,5-dihydro-2H-pyrrolo[3,2-c]pyridazine-7-carbonitrile;
Example 146: 4-(6-cyclopropylpyridin-3-yl)-5-isopropyl-2-(2-methyl-2H-indazol-5-yl)-3-oxo-3,5-dihydro-2H-pyrrolo[3,2-c]pyridazine-7-carbonitrile;
Example 147: 5-(cyclopropylmethyl)-4-(6-(difluoromethyl)pyridin-3-yl)-2-(1,2-dimethyl-1H-benzo[d]imidazol-6-yl)-3-oxo-3,5-dihydro-2H-pyrrolo[3,2-c]pyridazine-7-carbonitrile;
Example 148: 5-(cyclopropylmethyl)-4-(6-cyclopropylpyridin-3-yl)-2-(1,2-dimethyl-1H-benzo[d]imidazol-6-yl)-3-oxo-3,5-dihydro-2H-pyrrolo[3,2-c]pyridazine-7-carbonitrile;
Example 149: 4-(4-cyanophenyl)-5-(cyclopropylmethyl)-2-(2-methyl-2H-indazol-5-yl)-3-oxo-3,5-dihydro-2H-pyrrolo[3,2-c]pyridazine-7-carbonitrile;
Example 150: 5-(cyclopropylmethyl)-4-(4-cyclopropylphenyl)-2-(2-methyl-2H-indazol-5-yl)-2,5-dihydro-3H-pyrrolo[3,2-c]pyridazin-3-one;
Example 151: 5-(cyclopropylmethyl)-2-(1,2-dimethyl-1H-benzo[d]imidazol-6-yl)-4-(6-methylpyridin-3-yl)-3-oxo-3,5-dihydro-2H-pyrrolo[3,2-c]pyridazine-7-carbonitrile;
Example 152: 5-(cyclopropylmethyl)-2-(1,2-dimethyl-1H-benzo[d]imidazol-6-yl)-4-(6-ethylpyridin-3-yl)-3-oxo-3,5-dihydro-2H-pyrrolo[3,2-c]pyridazine-7-carbonitrile;
Example 153: 5-(5-(cyclopropylmethyl)-2-(2-methyl-2H-indazol-5-yl)-3-oxo-3,5-dihydro-2H-pyrrolo[3,2-c]pyridazin-4-yl)picolinonitrile;
Example 154: 4-(5-(cyclopropylmethyl)-2-(2-methyl-2H-indazol-5-yl)-3-oxo-3,5-dihydro-2H-pyrrolo[3,2-c]pyridazin-4-yl)benzonitrile;
Example 155: 5-(5-(cyclopropylmethyl)-2-(2-methyl-2H-indazol-5-yl)-3-oxo-3,5-dihydro-2H-pyrrolo[3,2-c]pyridazin-4-yl)picolinamide;
Example 156: 5-(cyclopropylmethyl)-4-(6-cyclopropylpyridin-3-yl)-2-(1,2-dimethyl-1H-benzo[d]imidazol-6-yl)-2,5-dihydro-3H-pyrrolo[3,2-c]pyridazin-3-one;
Example 157: 5-(cyclopropylmethyl)-4-(6-(hydroxymethyl)pyridin-3-yl)-2-(2-methyl-2H-indazol-5-yl)-2,5-dihydro-3H-pyrrolo[3,2-c]pyridazin-3-one;
Example 158: 5-(cyclopropylmethyl)-4-(6-(hydroxymethyl)pyridin-3-yl)-2-(2-methyl-2H-indazol-5-yl)-3-oxo-3,5-dihydro-2H-pyrrolo[3,2-c]pyridazine-7-carbonitrile;
Example 159: 5-(cyclobutylmethyl)-4-(6-cyclopropylpyridin-3-yl)-2-(2-methyl-2H-indazol-5-yl)-3-oxo-3,5-dihydro-2H-pyrrolo[3,2-c]pyridazine-7-carbonitrile;
Example 160: 5-(cyclopropylmethyl)-2-(2-(cyclopropylmethyl)-2H-indazol-5-yl)-4-(6-cyclopropylpyridin-3-yl)-3-oxo-3,5-dihydro-2H-pyrrolo[3,2-c]pyridazine-7-carbonitrile;
Example 161: 5-(cyclopropylmethyl)-4-(4-(methoxy-d3)phenyl)-2-(2-methyl-2H-indazol-5-yl)-3-oxo-3,5-dihydro-2H-pyrrolo[3,2-c]pyridazine-7-carbonitrile;
Example 162: 5-(cyclopropylmethyl)-4-(6-cyclopropylpyridin-3-yl)-N-methyl-2-(2-methyl-2H-indazol-5-yl)-3-oxo-2H,3H,5H-pyrrolopyridazine-7-carboxamide;
Example 163: 5-(cyclopropylmethyl)-4-(6-cyclopropylpyridin-3-yl)-2-(2-methyl-2H-indazol-5-yl)-3-oxo-3,5-dihydro-2H-pyrrolo[3,2-c]pyridazine-7-carboxylic acid;
Example 164: 5-(cyclopropylmethyl)-4-(6-cyclopropylpyridin-3-yl)-N,N-dimethyl-2-(2-methyl-2H-indazol-5-yl)-3-oxo-3,5-dihydro-2H-pyrrolo[3,2-c]pyridazine-7-carboxamide;
Example 165: 5-(cyclopropylmethyl)-4-(6-cyclopropylpyridin-3-yl)-2-(2-methyl-2H-indazol-5-yl)-3-oxo-3,5-dihydro-2H-pyrrolo[3,2-c]pyridazine-7-carboxamide;
Example 166: 5-(cyclopropylmethyl)-4-(4-cyclopropylphenyl)-2-(2-methyl-2H-indazol-5-yl)-3-oxo-3,5-dihydro-2H-pyrrolo[3,2-c]pyridazine-7-carboxamide;
Example 167: 5-(cyclopropylmethyl)-4-(4-cyclopropylphenyl)-N-methyl-2-(2-methyl-2H-indazol-5-yl)-3-oxo-3,5-dihydro-2H-pyrrolo[3,2-c]pyridazine-7-carboxamide;
Example 168: 5-(cyclopropylmethyl)-4-(4-(difluoromethoxy)phenyl)-2-(2-methyl-2H-indazol-5-yl)-3-oxo-3,5-dihydro-2H-pyrrolo[3,2-c]pyridazine-7-carboxamide;
Example 169: 5-(cyclopropylmethyl)-4-(4-(difluoromethoxy)phenyl)-N-methyl-2-(2-methyl-2H-indazol-5-yl)-3-oxo-3,5-dihydro-2H-pyrrolo[3,2-c]pyridazine-7-carboxamide;
Example 170: 4-(4-chlorophenyl)-5-(cyclopropylmethyl)-2-(2-methyl-2H-indazol-5-yl)-3-oxo-3,5-dihydro-2H-pyrrolo[3,2-c]pyridazine-7-carboxamide;
Example 171: 4-(4-chlorophenyl)-5-(cyclopropylmethyl)-N-methyl-2-(2-methyl-2H-indazol-5-yl)-3-oxo-3,5-dihydro-2H-pyrrolo[3,2-c]pyridazine-7-carboxamide;
Example 172: 5-(cyclopropylmethyl)-4-(6-cyclopropylpyridin-3-yl)-N-methyl-2-(2-(methyl-d3)-2H-indazol-5-yl)-3-oxo-3,5-dihydro-2H-pyrrolo[3,2-c]pyridazine-7-carboxamide;
Example 173: 5-(cyclopropylmethyl)-4-(4-(methoxy-d3)phenyl)-2-(2-methyl-2H-indazol-5-yl)-3-oxo-3,5-dihydro-2H-pyrrolo[3,2-c]pyridazine-7-carboxamide;
Example 174: 5-(cyclopropylmethyl)-4-(4-(methoxy-d3)phenyl)-N-methyl-2-(2-methyl-2H-indazol-5-yl)-3-oxo-3,5-dihydro-2H-pyrrolo[3,2-c]pyridazine-7-carboxamide;
Example 175: 5-(cyclopropylmethyl)-2-(2-methyl-2H-indazol-5-yl)-4-(6-methylpyridin-3-yl)-3-oxo-3,5-dihydro-2H-pyrrolo[3,2-c]pyridazine-7-carboxamide;
Example 176: 5-(cyclopropylmethyl)-4-(4-methoxyphenyl)-N-methyl-2-(2-methyl-2H-indazol-5-yl)-3-oxo-3,5-dihydro-2H-pyrrolo[3,2-c]pyridazine-7-carboxamide;
Example 177: 5-(cyclopropylmethyl)-N-methyl-2-(2-methyl-2H-indazol-5-yl)-4-(6-methylpyridin-3-yl)-3-oxo-3,5-dihydro-2H-pyrrolo[3,2-c]pyridazine-7-carboxamide;
Example 178: 5-(cyclopropylmethyl)-4-(6-cyclopropylpyridin-3-yl)-N,N-dimethyl-2-(2-methyl-2H-indazol-5-yl)-3-oxo-3,5-dihydro-2H-pyrrolo[3,2-c]pyridazine-7-sulfonamide;
Example 179: 5-(cyclopropylmethyl)-4-(4-cyclopropylphenyl)-N-methyl-2-(2-methyl-2H-indazol-5-yl)-3-oxo-3,5-dihydro-2H-pyrrolo[3,2-c]pyridazine-7-sulfonamide;
Example 180: 5-(cyclopropylmethyl)-4-(4-cyclopropylphenyl)-2-(2-methyl-2H-indazol-5-yl)-3-oxo-3,5-dihydro-2H-pyrrolo[3,2-c]pyridazine-7-sulfonamide;
Example 181: 5-(cyclopropylmethyl)-4-(6-cyclopropylpyridin-3-yl)-N-methyl-2-(2-methyl-2H-indazol-5-yl)-3-oxo-3,5-dihydro-2H-pyrrolo[3,2-c]pyridazine-7-sulfonamide;
Example 182: 5-(cyclopropylmethyl)-N-methyl-2-(2-methyl-2H-indazol-5-yl)-4-(6-methylpyridin-3-yl)-3-oxo-3,5-dihydro-2H-pyrrolo[3,2-c]pyridazine-7-sulfonamide;
Example 183: 5-(cyclopropylmethyl)-4-(4-(difluoromethoxy)phenyl)-N-methyl-2-(2-methyl-2H-indazol-5-yl)-3-oxo-3,5-dihydro-2H-pyrrolo[3,2-c]pyridazine-7-sulfonamide;
Example 184: 5-(cyclopropylmethyl)-4-(4-(difluoromethoxy)phenyl)-2-(2-methyl-2H-indazol-5-yl)-3-oxo-3,5-dihydro-2H-pyrrolo[3,2-c]pyridazine-7-sulfonamide;
Example 185: 5-(cyclopropylmethyl)-4-(6-cyclopropylpyridin-3-yl)-2-(2-methyl-2H-indazol-5-yl)-3-oxo-3,5-dihydro-2H-pyrrolo[3,2-c]pyridazine-7-sulfonamide;
Example 223: 5-(cyclopropylmethyl)-4-(4-(difluoromethoxy)phenyl)-2-(1-methyl-6-oxo-1,6-dihydropyridin-3-yl)-2,5-dihydro-3H-pyrrolo[3,2-c]pyridazin-3-one;
Example 224: 2-(benzo[d]thiazol-6-yl)-5-(cyclopropylmethyl)-4-(4-(difluoromethoxy)phenyl)-2,5-dihydro-3H-pyrrolo[3,2-c]pyridazin-3-one;
Example 225: 5-(cyclopropylmethyl)-4-(4-(difluoromethoxy)phenyl)-2-(quinolin-6-yl)-2,5-dihydro-3H-pyrrolo[3,2-c]pyridazin-3-one;
Example 226: 5-(cyclopropylmethyl)-4-(4-(difluoromethoxy)phenyl)-2-(pyridin-4-yl)-2,5-dihydro-3H-pyrrolo[3,2-c]pyridazin-3-one;
Example 227: 5-(cyclopropylmethyl)-4-(4-(difluoromethoxy)phenyl)-2-(4-methyl-3,4-dihydro-2H-benzo[b][1,4]oxazin-6-yl)-2,5-dihydro-3H-pyrrolo[3,2-c]pyridazin-3-one;
Example 228: 2-(4-aminophenyl)-5-(cyclopropylmethyl)-4-(4-(difluoromethoxy)phenyl)-2,5-dihydro-3H-pyrrolo[3,2-c]pyridazin-3-one;
Example 229: 5-(cyclopropylmethyl)-4-(4-(difluoromethoxy)phenyl)-2-(4-methoxyphenyl)-2,5-dihydro-3H-pyrrolo[3,2-c]pyridazin-3-one;
Example 230: 5-(cyclopropylmethyl)-4-(4-(difluoromethoxy)phenyl)-2-(4-methoxycyclohex-1-en-1-yl)-2,5-dihydro-3H-pyrrolo[3,2-c]pyridazin-3-one;
Example 231: 5-(cyclopropylmethyl)-4-(4-(difluoromethoxy)phenyl)-2-(1H-pyrazol-4-yl)-2,5-dihydro-3H-pyrrolo[3,2-c]pyridazin-3-one;
Example 232: 5-(cyclopropylmethyl)-4-(6-cyclopropylpyridin-3-yl)-2-(1-methyl-1,2,3,6-tetrahydropyridin-4-yl)-2,5-dihydro-3H-pyrrolo[3,2-c]pyridazin-3-one;
Example 233: 7-chloro-5-(cyclopropylmethyl)-4-(6-cyclopropylpyridin-3-yl)-2-(2-methyl-2H-indazol-5-yl)-2,5-dihydro-3H-pyrrolo[3,2-c]pyridazin-3-one;
Example 234: 5-(cyclopropylmethyl)-4-(6-cyclopropylpyridin-3-yl)-7-methyl-2-(2-methyl-2H-indazol-5-yl)-2,5-dihydro-3H-pyrrolo[3,2-c]pyridazin-3-one;
Example 235: 5-(cyclopropylmethyl)-4-(6-cyclopropylpyridin-3-yl)-7-fluoro-2-(2-methyl-2H-indazol-5-yl)-2,5-dihydro-3H-pyrrolo[3,2-c]pyridazin-3-one;
Example 236: 7-acetyl-5-(cyclopropylmethyl)-4-(6-cyclopropylpyridin-3-yl)-2-(2-methyl-2H-indazol-5-yl)-2,5-dihydro-3H-pyrrolo[3,2-c]pyridazin-3-one;
Example 237: 5-(cyclopropylmethyl)-7-fluoro-2-(2-methyl-2H-indazol-5-yl)-4-(6-methylpyridin-3-yl)-2,5-dihydro-3H-pyrrolo[3,2-c]pyridazin-3-one;
Example 238: 7-bromo-5-(cyclopropylmethyl)-4-(6-cyclopropylpyridin-3-yl)-2-(2-methyl-2H-indazol-5-yl)-2,5-dihydro-3H-pyrrolo[3,2-c]pyridazin-3-one;
Example 239: 5-(cyclopropylmethyl)-4-(6-cyclopropylpyridin-3-yl)-7-(hydroxymethyl)-2-(2-methyl-2H-indazol-5-yl)-2,5-dihydro-3H-pyrrolo[3,2-c]pyridazin-3-one;
Example 240: 5-(cyclopropylmethyl)-4-(6-cyclopropylpyridin-3-yl)-7-(difluoromethyl)-2-(2-methyl-2H-indazol-5-yl)-2,5-dihydro-3H-pyrrolo[3,2-c]pyridazin-3-one;
Example 241: 5-(cyclopropylmethyl)-4-(6-cyclopropylpyridin-3-yl)-2-(2-methyl-3-(trifluoromethyl)-2H-indazol-5-yl)-2,5-dihydro-3H-pyrrolo[3,2-c]pyridazin-3-one:
Example 242: 5-(cyclopropylmethyl)-4-(6-cyclopropylpyridin-3-yl)-2-(2-methyl-3-(trifluoromethyl)-2H-indazol-5-yl)-7-(trifluoromethyl)-2,5-dihydro-3H-pyrrolo[3,2-c]pyridazin-3-one;
Example 243: 5-(cyclopropylmethyl)-4-(6-cyclopropylpyridin-3-yl)-2-(2-methyl-2H-indazol-5-yl)-7-((trimethylsilyl)ethynyl)-2,5-dihydro-3H-pyrrolo[3,2-c]pyridazin-3-one;
Example 244: 5-(cyclopropylmethyl)-4-(6-cyclopropylpyridin-3-yl)-7-ethynyl-2-(2-methyl-2H-indazol-5-yl)-2,5-dihydro-3H-pyrrolo[3,2-c]pyridazin-3-one;
Example 245: 5-(cyclopropylmethyl)-4-(6-cyclopropylpyridin-3-yl)-2-(2-methyl-2H-indazol-5-yl)-7-(3,3,3-trifluoroprop-1-yn-1-yl)-2,5-dihydro-3H-pyrrolo[3,2-c]pyridazin-3-one;
Example 246: 5-(cyclopropylmethyl)-4-(6-cyclopropylpyridin-3-yl)-2-(2-methyl-2H-indazol-5-yl)-7-(trifluoromethyl)-2,5-dihydro-3H-pyrrolo[3,2-c]pyridazin-3-one;
Example 247: 5-(cyclopropylmethyl)-4-(6-cyclopropylpyridin-3-yl)-7-ethyl-2-(2-methyl-2H-indazol-5-yl)-2,5-dihydro-3H-pyrrolo[3,2-c]pyridazin-3-one;
Example 248: 5-(cyclopropylmethyl)-4-(6-cyclopropylpyridin-3-yl)-7-isopropyl-2-(2-methyl-2H-indazol-5-yl)-2,5-dihydro-3H-pyrrolo[3,2-c]pyridazin-3-one;
Example 249: (R)-5-(cyclopropylmethyl)-4-(6-cyclopropylpyridin-3-yl)-7-(1-hydroxyethyl)-2-(2-methyl-2H-indazol-5-yl)-2,5-dihydro-3H-pyrrolo[3,2-c]pyridazin-3-one;
Example 250: (S)-5-(cyclopropylmethyl)-4-(6-cyclopropylpyridin-3-yl)-7-(1-hydroxyethyl)-2-(2-methyl-2H-indazol-5-yl)-2,5-dihydro-3H-pyrrolo[3,2-c]pyridazin-3-one;
Example 251: 6-chloro-5-(cyclopropylmethyl)-4-(6-cyclopropylpyridin-3-yl)-2-(2-methyl-2H-indazol-5-yl)-3-oxo-3,5-dihydro-2H-pyrrolo[3,2-c]pyridazine-7-carbonitrile;
Example 252: (S)-5-(cyclopropylmethyl)-4-(6-(2,2-difluorocyclopropyl)pyridin-3-yl)-2-(2-methyl-2H-indazol-5-yl)-3-oxo-3,5-dihydro-2H-pyrrolo[3,2-c]pyridazine-7-carbonitrile;
Example 253: (R)-5-(cyclopropylmethyl)-4-(6-(2,2-difluorocyclopropyl)pyridin-3-yl)-2-(2-methyl-2H-indazol-5-yl)-3-oxo-3,5-dihydro-2H-pyrrolo[3,2-c]pyridazine-7-carbonitrile;
Example 254: 5-(cyclopropylmethyl)-2-(2-methyl-2H-indazol-5-yl)-4-[6-(1-methylcyclopropyl)pyridin-3-yl]-3-oxo-2H,3H,5H-pyrrolo[3,2-c]pyridazine-7-carbonitrile;
Example 255: 5-(cyclopropylmethyl)-7-(hydroxymethyl)-2-(2-methyl-2H-indazol-5-yl)-4-(6-methylpyridin-3-yl)-2,5-dihydro-3H-pyrrolo[3,2-c]pyridazin-3-one;
Example 256: 5-(cyclopropylmethyl)-4-(6-(1-hydroxycyclopropyl)pyridin-3-yl)-2-(2-methyl-2H-indazol-5-yl)-3-oxo-3,5-dihydro-2H-pyrrolo[3,2-c]pyridazine-7-carbonitrile;
Example 257: 5-(cyclopropylmethyl)-4-(6-(3-fluoroprop-1-en-2-yl)pyridin-3-yl)-2-(2-methyl-2H-indazol-5-yl)-3-oxo-3,5-dihydro-2H-pyrrolo[3,2-c]pyridazine-7-carbonitrile;
Example 258: 5-(cyclopropylmethyl)-2-(2-methyl-2H-indazol-5-yl)-4-(6-methylpyridin-3-yl)-7-(trifluoromethyl)-2,5-dihydro-3H-pyrrolo[3,2-c]pyridazin-3-one;
Example 259: 2-(5-(cyclopropylmethyl)-4-(6-cyclopropylpyridin-3-yl)-2-(2-methyl-2H-indazol-5-yl)-3-oxo-3,5-dihydro-2H-pyrrolo[3,2-c]pyridazin-7-yl)acetonitrile;
Example 261: 5-(cyclopropylmethyl)-4-(6-cyclopropylpyridin-3-yl)-2-(2-methyl-2H-indazol-5-yl)-7-(methylsulfonyl)-2,5-dihydro-3H-pyrrolo[3,2-c]pyridazin-3-one;
Example 262: 5-(cyclopropylmethyl)-4-(4-cyclopropylphenyl)-2-(2-methyl-2H-indazol-5-yl)-7-(methylsulfonyl)-2,5-dihydro-3H-pyrrolo[3,2-c]pyridazin-3-one;
Example 263: 5-(cyclopropylmethyl)-4-(6-cyclopropylpyridin-3-yl)-6-methyl-2-(2-methyl-2H-indazol-5-yl)-2,5-dihydro-3H-pyrrolo[3,2-c]pyridazin-3-one;
Example 264: 5-(cyclopropylmethyl)-4-(4-(difluoromethoxy)phenyl)-6-methyl-2-(2-methyl-2H-indazol-5-yl)-3-oxo-3,5-dihydro-2H-pyrrolo[3,2-c]pyridazine-7-carbonitrile;
Example 265: 5-(cyclopropylmethyl)-4-(6-cyclopropylpyridin-3-yl)-6-methyl-2-(2-methyl-2H-indazol-5-yl)-3-oxo-2H,3H,5H-pyrrolopyridazine-7-carbonitrile;
Example 266: 5-(cyclopropylmethyl)-6-methyl-2-(2-methyl-2H-indazol-5-yl)-4-(6-methylpyridin-3-yl)-3-oxo-2H,3H,5H-pyrrolopyridazine-7-carbonitrile;
Example 267: 5-(cyclopropylmethyl)-4-(6-cyclopropylpyridin-3-yl)-2-(2-methyl-2H-indazol-5-yl)-7-((methylamino)methyl)-2,5-dihydro-3H-pyrrolo[3,2-c]pyridazin-3-one;
Example 268: (S)-4-(6-cyclopropylpyridin-3-yl)-5-((2,2-difluorocyclopropyl)methyl)-2-(2-methyl-2H-indazol-5-yl)-3-oxo-3,5-dihydro-2H-pyrrolo[3,2-c]pyridazine-7-carbonitrile;
Example 269: (R)-4-(6-cyclopropylpyridin-3-yl)-5-((2,2-difluorocyclopropyl)methyl)-2-(2-methyl-2H-indazol-5-yl)-3-oxo-3,5-dihydro-2H-pyrrolo[3,2-c]pyridazine-7-carbonitrile; or
Example 270: 5-(cyclopropylmethyl)-4-(6-cyclopropylpyridin-3-yl)-2-(2-(methyl-d3)-2H-indazol-5-yl)-3-oxo-3,5-dihydro-2H-pyrrolo[3,2-c]pyridazine-7-carbonitrile;
   or a pharmaceutically acceptable salt thereof.

In other aspects, the compound of the application is:
Example 186: 5-(cyclopropylmethyl)-4-[4-(difluoromethoxy)phenyl]-2-(2-methyl-2H-indazol-5-yl)-2H,3H,5H-imidazo[4,5-c]pyridazin-3-one;
Example 187: 5-(cyclopropylmethyl)-2-(2-methyl-2H-indazol-5-yl)-4-(6-methylpyridin-3-yl)-2,5-dihydro-3H-imidazo[4,5-c]pyridazin-3-one;
Example 188: 4-(4-chlorophenyl)-5-(cyclopropylmethyl)-2-(2-methyl-2H-indazol-5-yl)-2,5-dihydro-3H-imidazo[4,5-c]pyridazin-3-one;
Example 189: 5-(cyclopropylmethyl)-4-(6-(difluoromethoxy)pyridin-3-yl)-2-(2-methyl-2H-indazol-5-yl)-2,5-dihydro-3H-imidazo[4,5-c]pyridazin-3-one;
Example 190: 5-(cyclopropylmethyl)-4-(6-cyclopropylpyridin-3-yl)-2-(2-methyl-2H-indazol-5-yl)-2,5-dihydro-3H-imidazo[4,5-c]pyridazin-3-one;
Example 191: 5-(cyclopropylmethyl)-4-(4-(difluoromethoxy)phenyl)-6-methyl-2-(2-methyl-2H-indazol-5-yl)-2,5-dihydro-3H-imidazo[4,5-c]pyridazin-3-one;
Example 192: 5-(cyclopropylmethyl)-4-(4-cyclopropylphenyl)-2-(2-methyl-2H-indazol-5-yl)-2,5-dihydro-3H-imidazo[4,5-c]pyridazin-3-one;
Example 193: 5-(cyclopropylmethyl)-4-(4-methoxyphenyl)-2-(2-methyl-2H-indazol-5-yl)-2,5-dihydro-3H-imidazo[4,5-c]pyridazin-3-one;
Example 194: 5-(cyclopropylmethyl)-4-(4-(methoxy-d3)phenyl)-2-(2-methyl-2H-indazol-5-yl)-2,5-dihydro-3H-imidazo[4,5-c]pyridazin-3-one; or
Example 195: 5-(cyclopropylmethyl)-2-(1,2-dimethyl-1H-benzo[d]imidazol-6-yl)-4-(4-methoxyphenyl)-2,5-dihydro-3H-imidazo[4,5-c]pyridazin-3-one;
   or a pharmaceutically acceptable salt thereof.

In further aspects, the compound of the application is:
Example 196: 5-(cyclopropylmethyl)-4-[4-(difluoromethoxy)phenyl]-7-methyl-2-(2-methyl-2H-indazol-5-yl)-2H,3H,5H,6H,7H-imidazolidino[4,5-c]pyridazine-3,6-dione;
Example 197: 5-(cyclopropylmethyl)-4-(4-(difluoromethoxy)phenyl)-2-(2-methyl-2H-indazol-5-yl)-2,7-dihydro-3H-imidazo[4,5-c]pyridazine-3,6(5H)-dione;
Example 198: 5-(cyclopropylmethyl)-4-(6-cyclopropylpyridin-3-yl)-7-methyl-2-(2-methyl-2H-indazol-5-yl)-2,7-dihydro-3H-imidazo[4,5-c]pyridazine-3,6(5H)-dione;
Example 199: 4-(4-chlorophenyl)-5-(cyclopropylmethyl)-7-methyl-2-(2-methyl-2H-indazol-5-yl)-2,7-dihydro-3H-imidazo[4,5-c]pyridazine-3,6(5H)-dione;
Example 200: 5-(cyclopropylmethyl)-4-(4-cyclopropylphenyl)-7-methyl-2-(2-methyl-2H-indazol-5-yl)-2,7-dihydro-3H-imidazo[4,5-c]pyridazine-3,6(5H)-dione;
Example 201: 5-(cyclopropylmethyl)-4-(4-(methoxy-d3)phenyl)-7-methyl-2-(2-methyl-2H-indazol-5-yl)-2,7-dihydro-3H-imidazo[4,5-c]pyridazine-3,6(5H)-dione;
Example 202: 5-(cyclopropylmethyl)-7-methyl-2-(2-methyl-2H-indazol-5-yl)-4-(6-methylpyridin-3-yl)-2,7-dihydro-3H-imidazo[4,5-c]pyridazine-3,6(5H)-dione;
Example 203: 5-(cyclopropylmethyl)-4-(6-cyclopropylpyridin-3-yl)-2-(2-methyl-2H-indazol-5-yl)-2,7-dihydro-3H-imidazo[4,5-c]pyridazine-3,6(5H)-dione;
Example 204: 5-(cyclopropylmethyl)-4-(4-cyclopropylphenyl)-2-(2-methyl-2H-indazol-5-yl)-2,7-dihydro-3H-imidazo[4,5-c]pyridazine-3,6(5H)-dione;
Example 205: 4-(4-chlorophenyl)-5-(cyclopropylmethyl)-2-(2-methyl-2H-indazol-5-yl)-2,7-dihydro-3H-imidazo[4,5-c]pyridazine-3,6(5H)-dione;
Example 206: 5-(cyclopropylmethyl)-4-(4-(methoxy-d3)phenyl)-2-(2-methyl-2H-indazol-5-yl)-2,7-dihydro-3H-imidazo[4,5-c]pyridazine-3,6(5H)-dione; or
Example 207: 5-(cyclopropylmethyl)-2-(2-methyl-2H-indazol-5-yl)-4-(6-methylpyridin-3-yl)-2,7-dihydro-3H-imidazo[4,5-c]pyridazine-3,6(5H)-dione; or a pharmaceutically acceptable salt thereof.

In yet other aspects, the compound of the application is:
Example 208: 1-(cyclopropylmethyl)-7-(4-(difluoromethoxy)phenyl)-5-(2-methyl-2H-indazol-5-yl)-1,5-dihydro-6H-[1,2,3]triazolo[4,5-c]pyridazin-6-one;
Example 209: 1-(cyclopropylmethyl)-7-(4-cyclopropylphenyl)-5-(2-methyl-2H-indazol-5-yl)-1,5-dihydro-6H-[1,2,3]triazolo[4,5-c]pyridazin-6-one; or
Example 210 1-(cyclopropylmethyl)-7-(6-cyclopropylpyridin-3-yl)-5-(2-methyl-2H-indazol-5-yl)-1,5-dihydro-6H-[1,2,3]triazolo[4,5-c]pyridazin-6-one;
   or a pharmaceutically acceptable salt thereof.

In still further aspects, the compound of the application is:
Example 211: 1-(cyclopropylmethyl)-7-(6-cyclopropylpyridin-3-yl)-3-methyl-5-(2-methyl-2H-indazol-5-yl)-1H,5H,6H-pyrazolo[4,3-c]pyridazin-6-one;
Example 212: 1-(cyclopropylmethyl)-7-(4-(difluoromethoxy)phenyl)-3-methyl-5-(2-methyl-2H-indazol-5-yl)-1,5-dihydro-6H-pyrazolo[4,3-c]pyridazin-6-one;
Example 213: 1-(cyclopropylmethyl)-7-(4-cyclopropylphenyl)-3-methyl-5-(2-methyl-2H-indazol-5-yl)-1, 5-dihydro-6H-pyrazolo[4,3-c]pyridazin-6-one;
Example 214: 1-(cyclopropylmethyl)-7-(4-methoxyphenyl)-3-methyl-5-(2-methyl-2H-indazol-5-yl)-1, 5-dihydro-6H-pyrazolo[4,3-c]pyridazin-6-one;
Example 215: 7-(4-chlorophenyl)-1-(cyclopropylmethyl)-3-methyl-5-(2-methyl-2H-indazol-5-yl)-1,5-dihydro-6H-pyrazolo[4,3-c]pyridazin-6-one;
Example 216: 1-(cyclopropylmethyl)-7-(4-(methoxy-d3)phenyl)-3-methyl-5-(2-methyl-2H-indazol-5-yl)-1, 5-dihydro-6H-pyrazolo[4,3-c]pyridazin-6-one;
Example 217: 1-(cyclopropylmethyl)-3-methyl-5-(2-methyl-2H-indazol-5-yl)-7-(6-methylpyridin-3-yl)-1, 5-dihydro-6H-pyrazolo[4,3-c]pyridazin-6-one;
Example 218: 1-(cyclopropylmethyl)-7-(4-(difluoromethoxy)phenyl)-5-(2-methyl-2H-indazol-5-yl)-1, 5-dihydro-6H-pyrazolo[4,3-c]pyridazin-6-one;
Example 219: 1-(cyclopropylmethyl)-7-(6-cyclopropylpyridin-3-yl)-5-(2-methyl-2H-indazol-5-yl)-1, 5-dihydro-6H-pyrazolo[4,3-c]pyridazin-6-one;
Example 220: 1-(cyclopropylmethyl)-7-(4-(methoxy-d3)phenyl)-5-(2-methyl-2H-indazol-5-yl)-1, 5-dihydro-6H-pyrazolo[4,3-c]pyridazin-6-one;
Example 221: 7-(4-chlorophenyl)-1-(cyclopropylmethyl)-5-(2-methyl-2H-indazol-5-yl)-1,5-dihydro-6H-pyrazolo[4,3-c]pyridazin-6-one; or
Example 222: 1-(cyclopropylmethyl)-5-(2-methyl-2H-indazol-5-yl)-7-(6-methylpyridin-3-yl)-1, 5-dihydro-6H-pyrazolo[4,3-c]pyridazin-6-one;
   or a pharmaceutically acceptable salt thereof.

In other aspects, the compound of the application is:
Example 260: 5-(cyclopropylmethyl)-4-(6-cyclopropylpyridin-3-yl)-7,7-dimethyl-2-(2-methyl-2H-indazol-5-yl)-2,5,6,7-tetrahydro-3H-pyrrolo[3,2-c]pyridazin-3-one;
   or a pharmaceutically acceptable salt thereof.

In further aspects, the compound of the application is:
Example 271: 5-(cyclopropylmethyl)-4-(4-(difluoromethoxy)phenyl)-2-(2-methyl-2H-indazol-5-yl)-2,7-dihydro-3H-pyrrolo[2,3-c]pyridazin-3-one; or Example 272: 5-(cyclopropylmethyl)-4-(4-(difluoromethoxy)phenyl)-7-methyl-2-(2-methyl-2H-indazol-5-yl)-2,7-dihydro-3H-pyrrolo[2,3-c]pyridazin-3-one;
   or a pharmaceutically acceptable salt thereof.

In still other aspects, the compound of the application is:

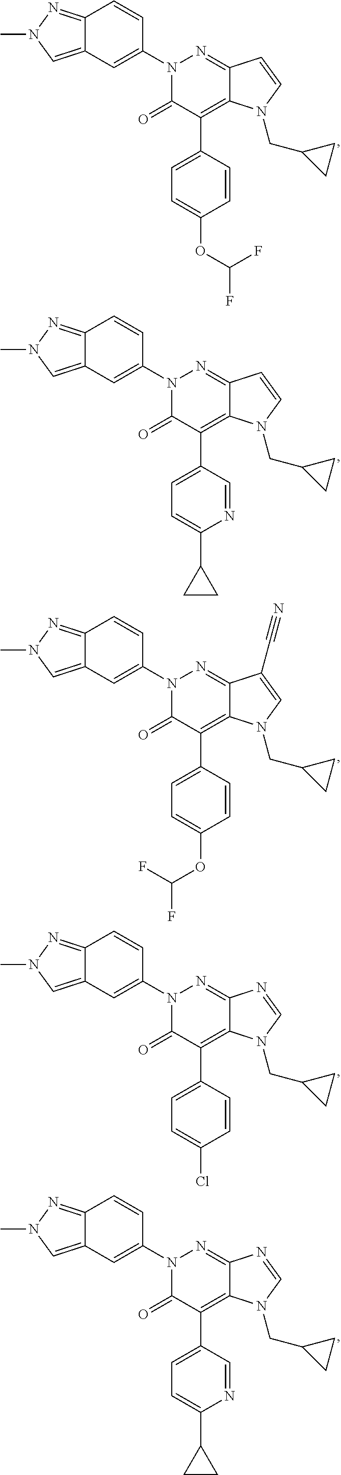

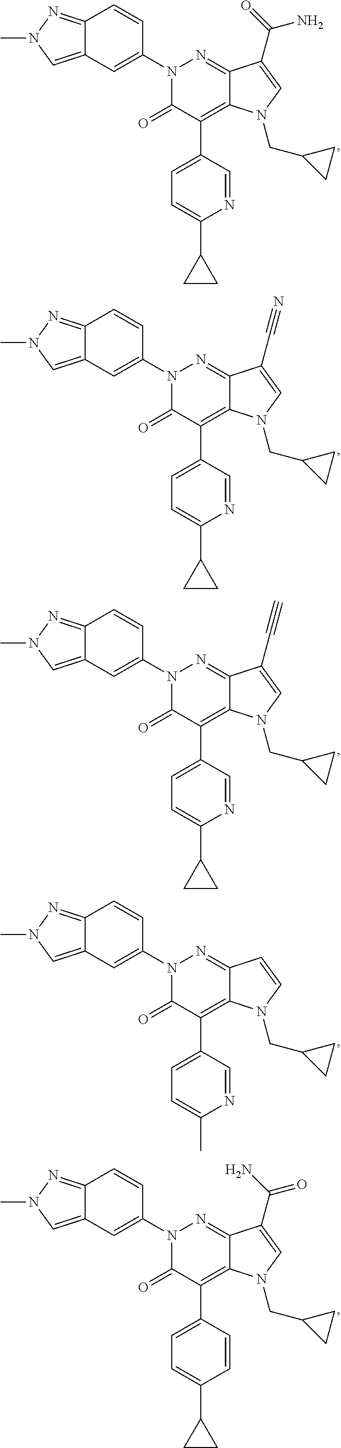 [SMILES: Cn1cc2cc(-n3nc4c(C(N)=O)cn(CC5CC5)c4c(-c4ccc(C5CC5)nc4)c3=O)ccc2n1 ; Cn1cc2cc(-n3nc4c(C#N)cn(CC5CC5)c4c(-c4ccc(C5CC5)nc4)c3=O)ccc2n1 ; C#Cc1cn(CC2CC2)c2c(-c3ccc(C4CC4)nc3)c(=O)n(-c3ccc4nn(C)cc4c3)nc12 ; Cc1ccc(-c2c(=O)n(-c3ccc4nn(C)cc4c3)nc3ccn(CC4CC4)c23)cn1 ; Cn1cc2cc(-n3nc4c(C(N)=O)cn(CC5CC5)c4c(-c4ccc(C5CC5)cc4)c3=O)ccc2n1]

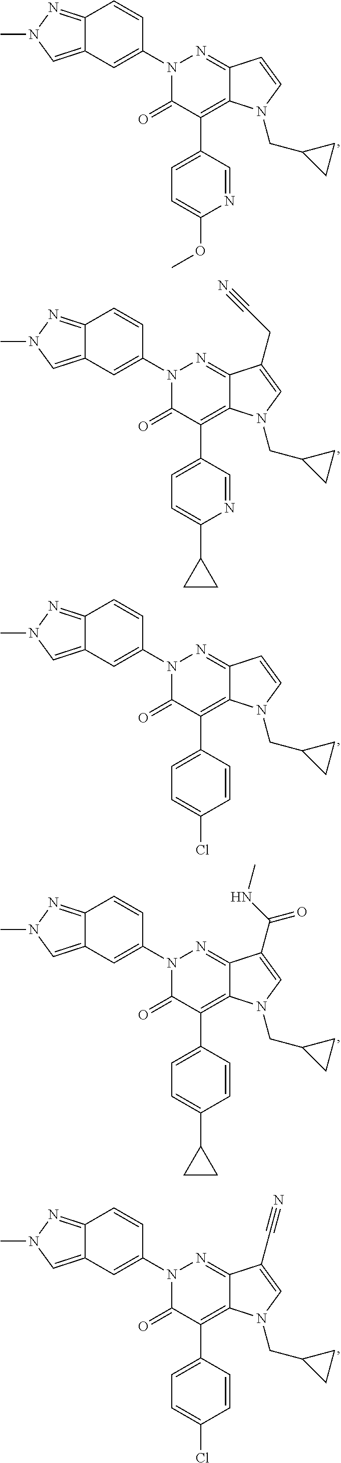

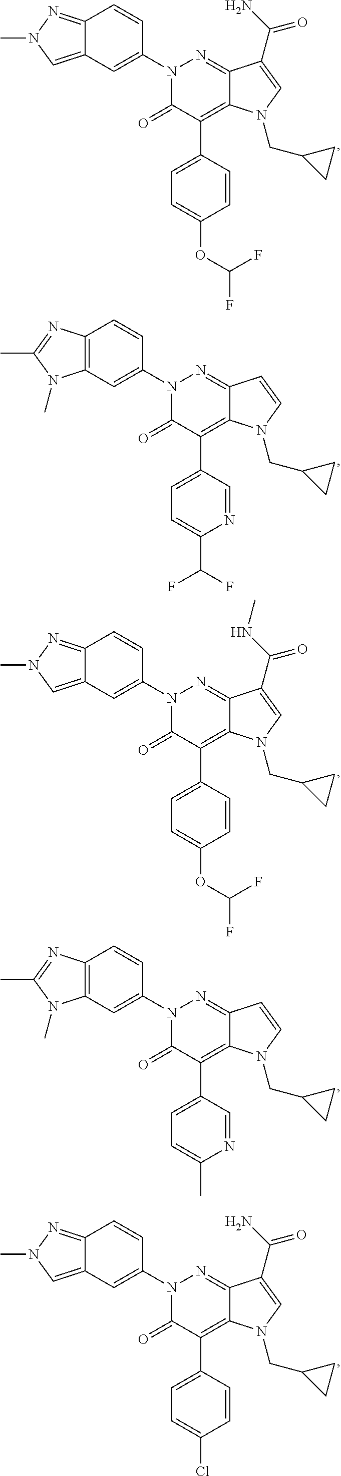

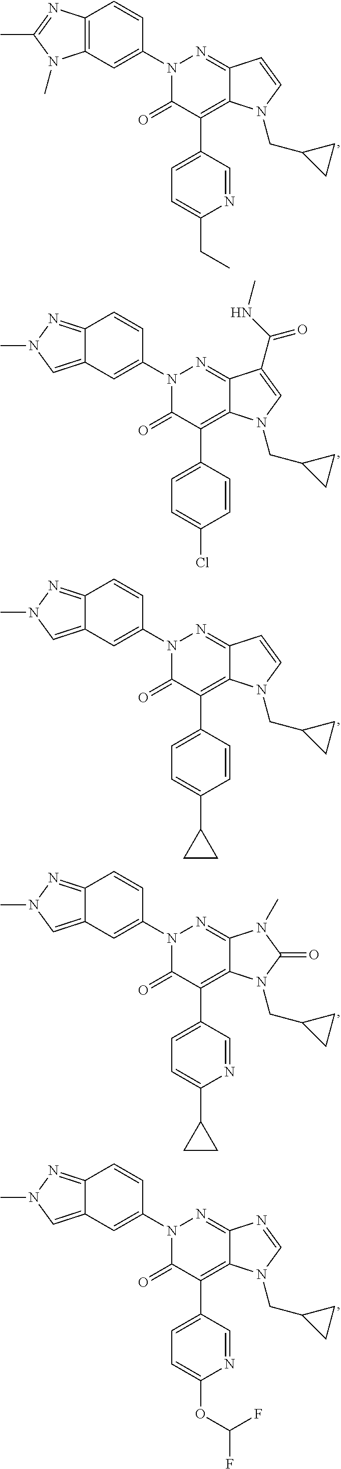

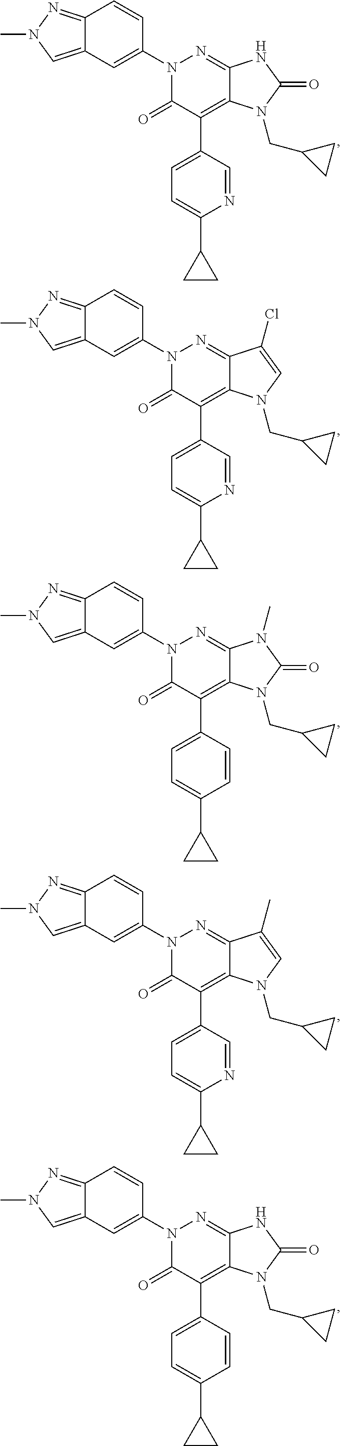 [SMILES: Cn1cc2cc(-n3nc4[nH]c(=O)n(CC5CC5)c4c(-c4ccc(C5CC5)nc4)c3=O)ccc2n1 ; Cn1cc2cc(-n3nc4c(Cl)cn(CC5CC5)c4c(-c4ccc(C5CC5)nc4)c3=O)ccc2n1 ; Cn1cc2cc(-n3nc4c(c(-c5ccc(C6CC6)cc5)c3=O)n(CC3CC3)c(=O)n4C)ccc2n1 ; Cc1cn(CC2CC2)c2c(-c3ccc(C4CC4)nc3)c(=O)n(-c3ccc4nn(C)cc4c3)nc12 ; Cn1cc2cc(-n3nc4[nH]c(=O)n(CC5CC5)c4c(-c4ccc(C5CC5)cc4)c3=O)ccc2n1]

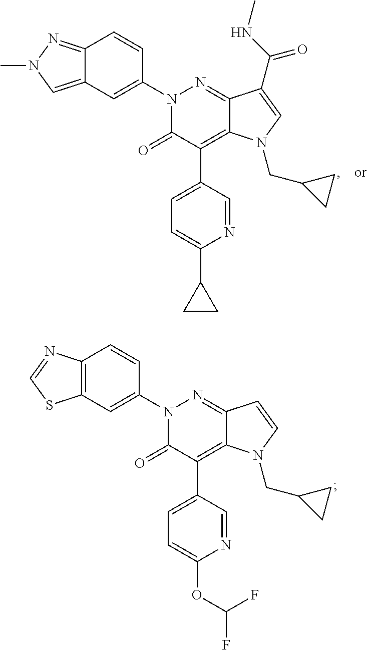 [SMILES: CNC(=O)c1cn(CC2CC2)c2c(-c3ccc(C4CC4)nc3)c(=O)n(-c3ccc4nn(C)cc4c3)nc12 ; O=c1c(-c2ccc(OC(F)F)nc2)c2c(ccn2CC2CC2)nn1-c1ccc2ncsc2c1]

or a pharmaceutically acceptable salt thereof.

“Pharmaceutically acceptable salts” include both acid and base addition salts. In certain embodiments, the compounds of the present application are in the form of a pharmaceutically acceptable salt. “Pharmaceutically acceptable acid addition salt” refers to those salts which retain the biological effectiveness and properties of the free bases and which are not biologically or otherwise undesirable, formed with inorganic acids such as hydrochloric acid, hydrobromic acid, sulfuric acid, nitric acid, carbonic acid, phosphoric acid and the like, and organic acids may be selected from aliphatic, cycloaliphatic, aromatic, araliphatic, heterocyclic, carboxylic, and sulfonic classes of organic acids such as formic acid, acetic acid, propionic acid, glycolic acid, gluconic acid, lactic acid, pyruvic acid, oxalic acid, malic acid, maleic acid, malonic acid, succinic acid, fumaric acid, tartaric acid, citric acid, aspartic acid, ascorbic acid, glutamic acid, anthranilic acid, benzoic acid, cinnamic acid, mandelic acid, embonic acid, phenylacetic acid, methanesulfonic acid, ethanesulfonic acid, p-toluenesulfonic acid, salicylic acid and the like. “Pharmaceutically acceptable base addition salts” include those derived from inorganic bases such as sodium, potassium, lithium, ammonium, calcium, magnesium, iron, zinc, copper, manganese, aluminum salts and the like. In some embodiments, base addition salts are the ammonium, potassium, sodium, calcium and magnesium salts. Salts derived from pharmaceutically acceptable organic nontoxic bases includes salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-diethylaminoethanol, trimethamine, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins and the like. Particularly organic non-toxic bases are isopropylamine, diethylamine, ethanolamine, trimethamine, dicyclohexylamine, choline, and caffeine.

In some embodiments, the compound is a salt. In other embodiments, the compound is a pharmaceutically acceptable salt. In further embodiments, the compound is an acetate. In yet other embodiments, the compound is a benzoate salt. In still further embodiments, the compound is a besylate salt. In other embodiments, the compound is a bitartrate salt. In further embodiments, the compound is a bromide salt. In still other embodiments, the compound is a carbonate salt. In yet further embodiments, the compound is a chloride salt. In other embodiments, the compound of the present application is a citrate salt. In further embodiments, the compound is an edetate salt. In yet other embodiments, the compound is an edisylate salt. In still further embodiments, the compound is an estolate salt. In other embodiments, the compound is a fumarate salt. In further embodiments, the compound is a gluceptate salt. In yet other embodiments, the compound is a gluconate salt. In still further embodiments, the compound is a hydrobromide salt. In still other embodiments, the compound is a hydrochloride salt. In yet further embodiments, the compound is an iodide salt. In other embodiments, the compound is a lactate salt. In other embodiments, the compound is a lactobionate salt. In further embodiments, the compound is a malate salt. In yet other embodiments, the compound is a maleate salt. In still further embodiments, the compound is a mandelate salt. In other embodiments, the compound is a mesylate salt. In further embodiments, the compound is a methyl bromide salt. In still other embodiments, the compound is a methyl sulfate salt. In yet further embodiments, the compound is a napsylate salt. In other embodiments, the compound is a nitrate salt. In further embodiments, the compound is a pamoate salt. In yet other embodiments, the compound is a phosphate salt. In still further embodiments, the compound is a diphosphate salt. In other embodiments, the compound is a salicylate salt. In further embodiments, the compound is a disalicylate salt. In still other embodiments, the compound is a stearate salt. In yet further embodiments, the compound is a succinate salt. In other embodiments, the compound is a sulfate salt. In further embodiments, the compound is a tartrate salt. In yet other embodiments, the compound is a tosylate salt. In still further embodiments, the compound is a triethiodide salt. In other embodiments, the compound is a valerate salt. In further embodiments, the compound is an aluminum salt. In still other embodiments, the compound is a benzathine salt. In yet further embodiments, the compound is a calcium salt. In other embodiments, the compound is a ethylenediamine salt. In further embodiments, the compound is a lysine salt. In yet other embodiments, the compound is a magnesium salt. In still further embodiments, the compound is a meglumine salt. In other embodiments, the compound is a potassium salt. In further embodiments, the compound is a procaine salt. In still other embodiments, the compound is a sodium salt. In further embodiments, the compound is a tromethamine salt. In still other embodiments, the compound is a zinc salt.

Compounds of the present application may exist in different tautomeric forms. In some embodiments, the compounds are in the form as drawn or named. In other embodiments, the compounds are in a tautomeric form other than as drawn or named.

Compounds of the present application may exist as one or a mixture of salts and solvate forms. For example, the compound may be substantially pure in one particular salt or solvate form or else may be mixtures of two or more salt or solvate forms. In some embodiments, the compounds are in solvate form. In other embodiments, the compounds exist as hydrates.

The present application also encompasses prodrugs of the compounds described above. Suitable prodrugs where applicable include known amino-protecting and carboxy-protecting groups which are released, for example hydrolyzed, to yield the parent compound under physiologic conditions. A particular class of prodrugs are compounds in which a nitrogen atom in an amino, amidino, aminoalkyleneamino, iminoalkyleneamino or guanidino group is substituted with a hydroxy (OH) group, an alkylcarbonyl (—C(O)—R′″) group, an alkoxycarbonyl (—C(O)—OR′″), an acyloxyalkyl-alkoxycarbonyl (—C(O)—O—R′″—O—C(O)—R′″) group where R′″ is a alkyl, aryl, carbocyclyl, heteroaryl, heterocyclyl, or a group having the formula —C(O)—O—CP₁P₂-haloalkyl, where P₁ and P₂ are the same or different and are H, lower alkyl, lower alkoxy, cyano, halo, or aryl. In some embodiments, the nitrogen atom is one of the nitrogen atoms of the amidino group of the compounds of the present application. Prodrug compounds are prepared reacting the compounds of the present application with an activated acyl compound to bond a nitrogen atom in the compound of the present application to the carbonyl of the activated acyl compound. Suitable activated carbonyl compounds contain a good leaving group bonded to the carbonyl carbon and include acyl halides, acyl amines, acyl pyridinium salts, acyl alkoxides, e.g., acyl phenoxides such as p-nitrophenoxy acyl, dinitrophenoxy acyl, fluorophenoxy acyl, and difluorophenoxy acyl. The reactions are carried out in inert solvents at reduced temperatures such as −78 to about 50° C. The reactions are usually also carried out in the presence of an inorganic base such as potassium carbonate or sodium bicarbonate, or an organic base such as an amine, including pyridine, triethylamine, etc. One manner of preparing prodrugs is described in International Patent Publication No. WO-98/46576, the content of which is incorporated herein by reference in its entirety.

Compounds of the present application may exist in different resonance forms and that all such resonance forms are within the scope of the present application herein.

### Preparation Methods

Compounds of the present application, or pharmaceutically acceptable salts thereof, are prepared using standard organic synthetic techniques from commercially available starting materials and reagents. It will be appreciated that synthetic procedures employed in the preparation of compounds of the present application, or pharmaceutically acceptable salts thereof, will depend on the particular substituents present in a compound and that various protection and deprotection steps that are standard in organic synthesis may be required but may not be illustrated in the following general schemes.

In some embodiments, the compounds of the present application, or pharmaceutically acceptable salts thereof, may be prepared by the general procedure I.

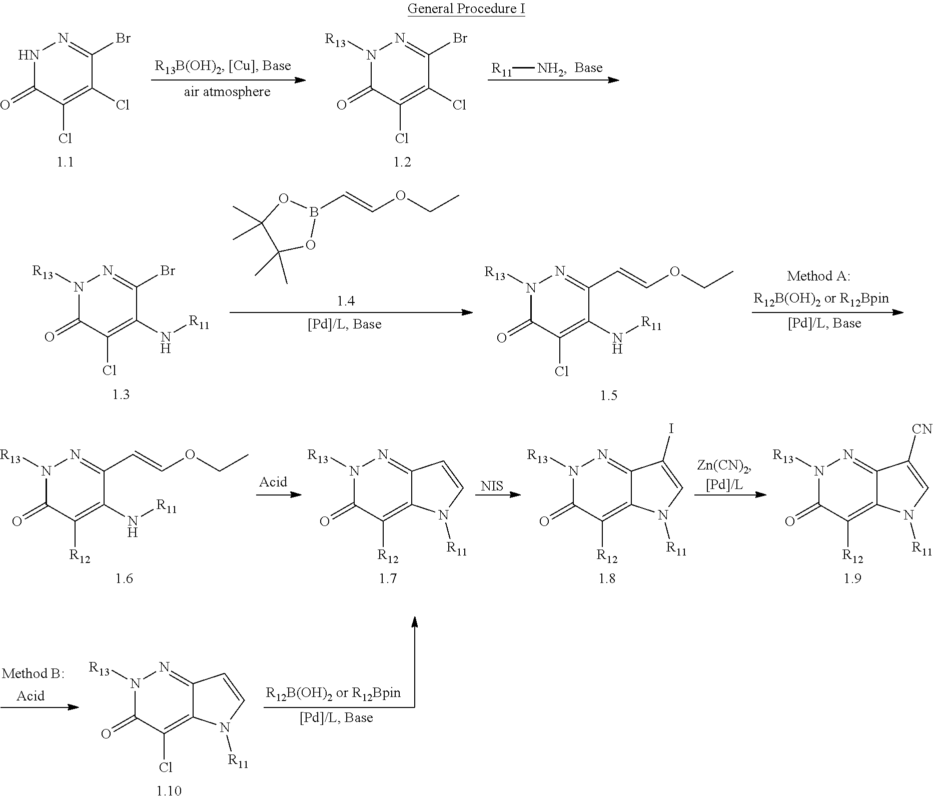

Compounds of structure 1.7 and 1.9 were obtained through the scheme depicted as General Procedure I. Beginning with pyridazinone 1.1, the desired R₁₃ group was installed using a Chan-Lam coupling to generate compound 1.2. A nucleophilic substitution reaction was then used to install the desired Rn group and generate compound 1.3. Reagent 1.4 was used in a Suzuki-coupling reaction to generate compound 1.5, which was then subjected to another Suzuki-coupling to install the desired R₁₂ group as compound 1.6. Compound 1.6 was then subjected to acid to close the five-member ring and generate compound 1.7 (Method A). Alternatively, compound 1.5 was subjected to acid to close the five-member ring and generate compound 1.10 followed by a Suzuki-coupling to install the desired R₁₂ group and generate compound 1.7 (Method B). Final compounds of structure 1.7 could be further reacted with an electrophilic iodide source to generate compound 1.8, which was then reacted in a Negishi-coupling reaction to afford final compounds of structure 1.9.

In some embodiments, the compounds of the present application, or pharmaceutically acceptable salts thereof, may be prepared by the general procedure II.

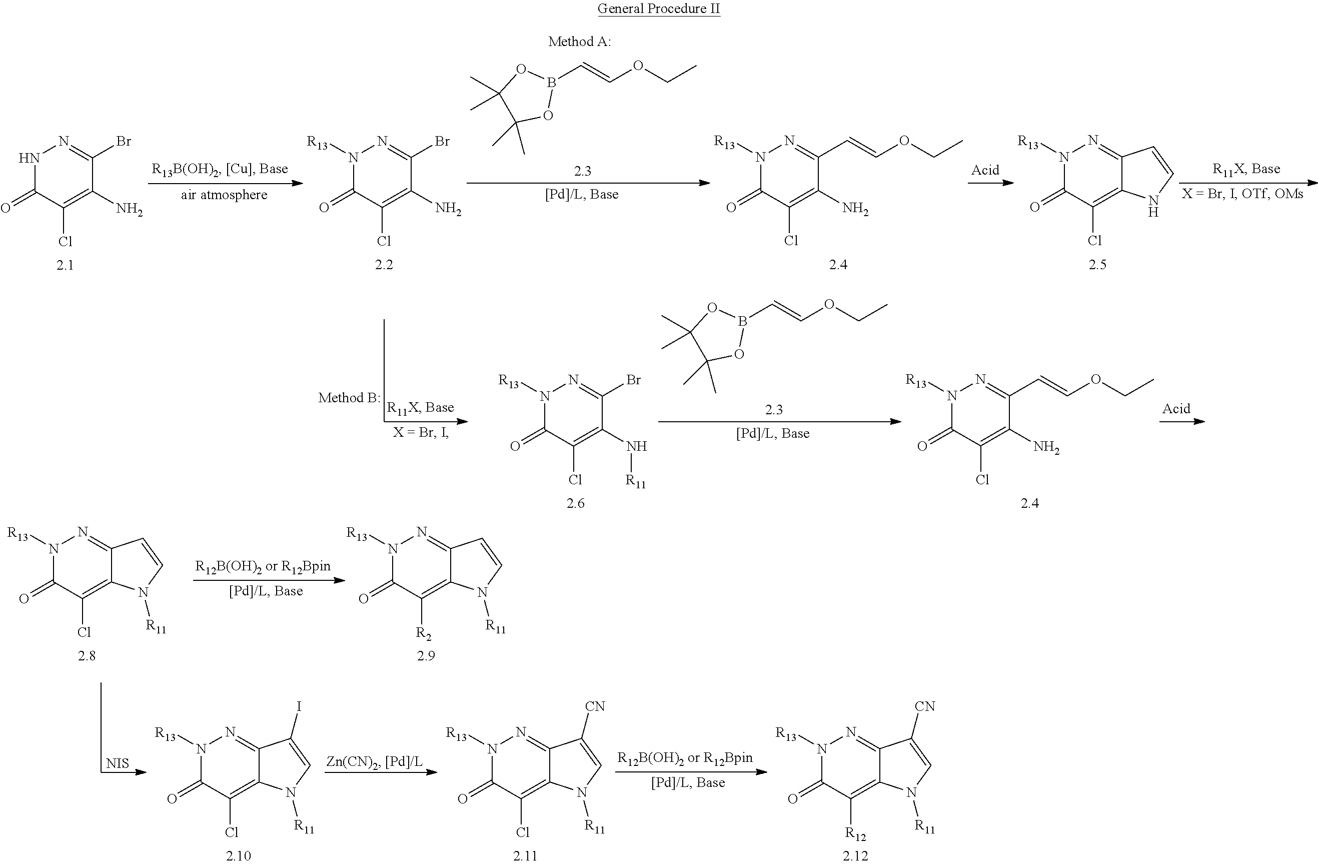

Compounds of structure 2.9 and 2.12 were obtained through the scheme depicted as General Procedure II. Beginning with pyridazinone 2.1, the desired R₁₃ group was installed using a Chan-Lam coupling to generate compound 2.2. Ethyl vinyl ether was then introduced through a suzuki coupling to generate intermediate 2.4 which was cyclized to bicyclic compound 2.5 after exposure to acid. The desired R₁₁ group was introduced through N-alkylation of the pyrrole ring system to generate compound 2.8 (Method A). Alternatively, the desired R₁₁ group was introduced through the N-alkylation of compound 2.2 to generate compound 2.6. Ethyl vinyl ether was then introduced by suzuki coupling to generate compound 2.7, which was then cyclized to compound 2.8 through an acid-mediated ring closure (Method B). The desired R₁₂ group was introduced through a suzuki coupling to afford final compounds of structure 2.9. Alternatively, compound 2.8 was iodinated to generate compound 2.10 and then reacted with Zn(CN)₂ to generate compound 2.11. The desired R₁₂ group was introduced through a suzuki coupling to afford final compounds of structure 2.12.

In some embodiments, the compounds of the present application, or pharmaceutically acceptable salts thereof, may be prepared by the general procedure III.

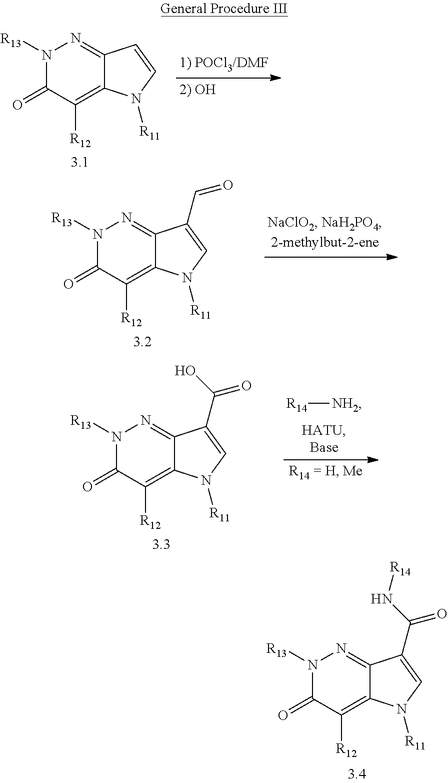

Compounds of structure 3.4 were obtained through the scheme depicted as General Procedure III. Intermediate 3.1 was subjected to the Vilsmeier-Haack reaction to generate aldehyde 3.2. Compound 3.2 was then subjected to a Pinnick oxidation to generate carboxylic acid 3.3. The desired R₁₄ group was introduced through an amide coupling reaction to afford final compounds of structure 3.4.

In some embodiments, the compounds of the present application, or pharmaceutically acceptable salts thereof, may be prepared by the general procedure IV.

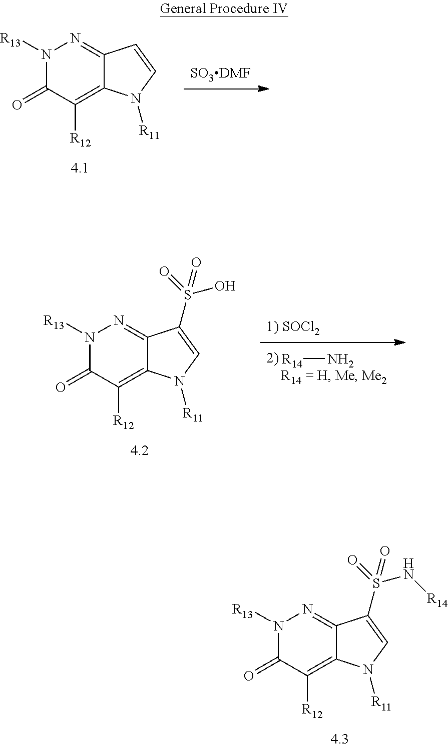

Compounds of structure 4.3 were obtained through the scheme depicted as General Procedure IV. Substituted intermediate 4.1 was subjected to SO₃ to generate sulfonic acid 4.2. Compound 4.2 was treated with SOCl₂ and then subjected to an amine substituted with the desired R₁₄ group to afford final compounds of structure 4.3.

In some embodiments, the compounds of the present application, or pharmaceutically acceptable salts thereof, may be prepared by the general procedure V.

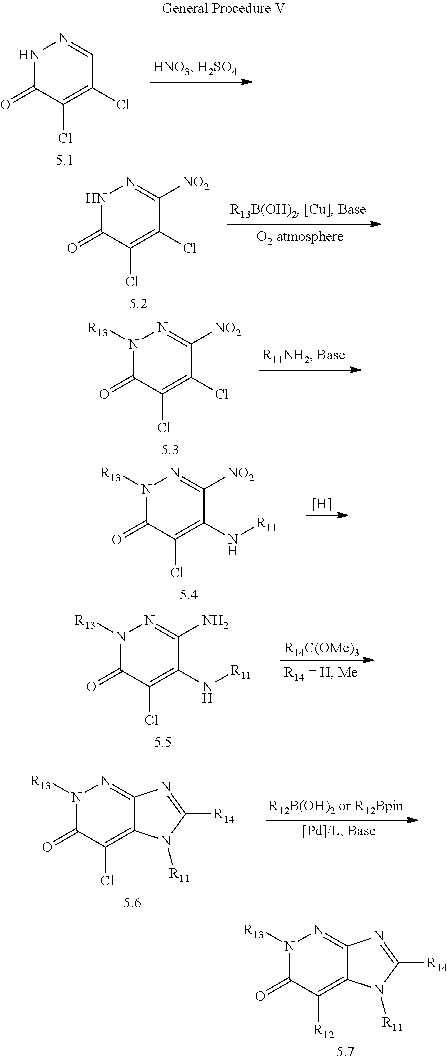

Compounds of structure 5.7 were obtained through the scheme depicted as General Procedure V. Starting with pyridazinone 5.1 the heterocycle was nitrated to afford pyridazinone 5.2. The desired R₁₃ group was installed using a Chan-Lam coupling to generate compound 5.3. The desired Rn group was introduced through a nucleophilic aromatic substitution to generate amino-heterocycle 5.4. The nitro group was selectively reduced to generate compound 5.5. The bicyclic core was formed through a condensation reaction with either trimethyl-orthoformate or trimethyl-orthoacetate, depending on the desired R₁₄ group, to generate compound 5.6. The desired R₁₂ group was introduced through a suzuki coupling to afford final compounds of structure 5.7.

In some embodiments, the compounds of the present application, or pharmaceutically acceptable salts thereof, may be prepared by the general procedure VI.

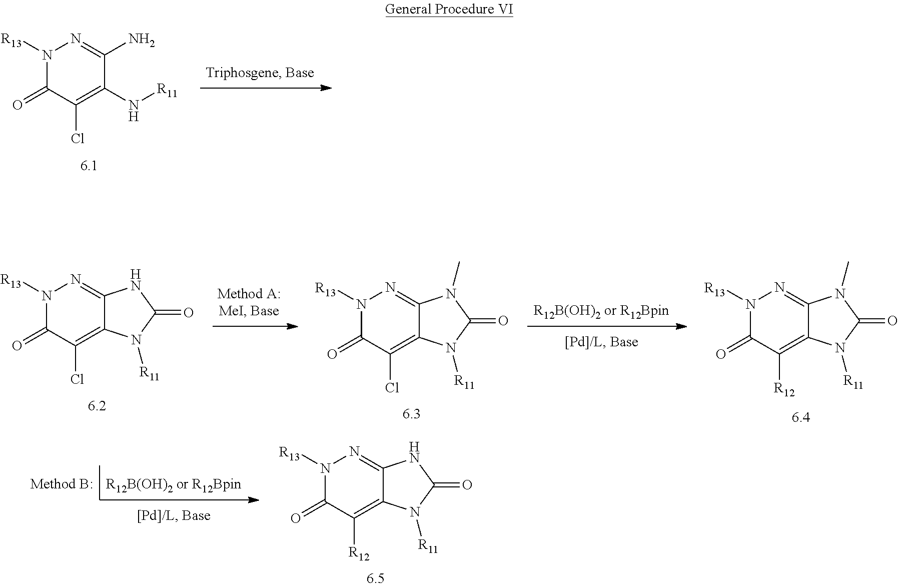

Compounds of structure 6.4 and 6.5 were obtained through the scheme depicted as General Procedure VI. Beginning with intermediate 6.1, the bicyclic ring system was closed through a reaction with triphosgene to generate compound 6.2. The 2-imidazolidinone was methylated to generate compound 6.3, and the desired R₁₂ group was installed using a Suzuki coupling to afford final compounds of structure 6.4 (Method A). Alternatively, the desired R₁₂ group could be installed using a Suzuki coupling on compound 6.2 to afford final compounds of structure 6.5 (Method B).

In some embodiments, the compounds of the present application, or pharmaceutically acceptable salts thereof, may be prepared by the general procedure VII.

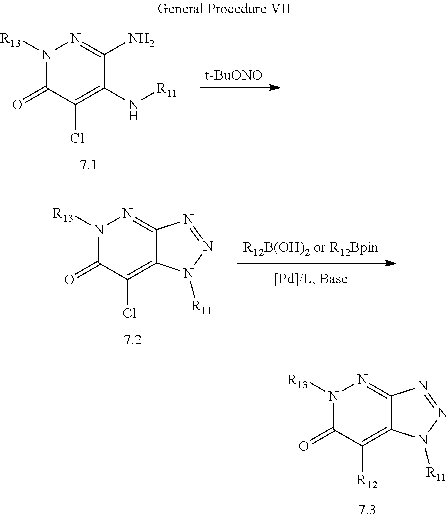

Compounds of structure 7.3 were obtained through the scheme depicted as General Procedure VII. Intermediate 7.1 was exposed to tert-butyl nitrite to generate bicyclic triazole 7.2. The desired R₁₂ group was then introduced through a suzuki coupling to afford final compounds of structure 7.3.

In some embodiments, the compounds of the present application, or pharmaceutically acceptable salts thereof, may be prepared by the general procedure VIII.

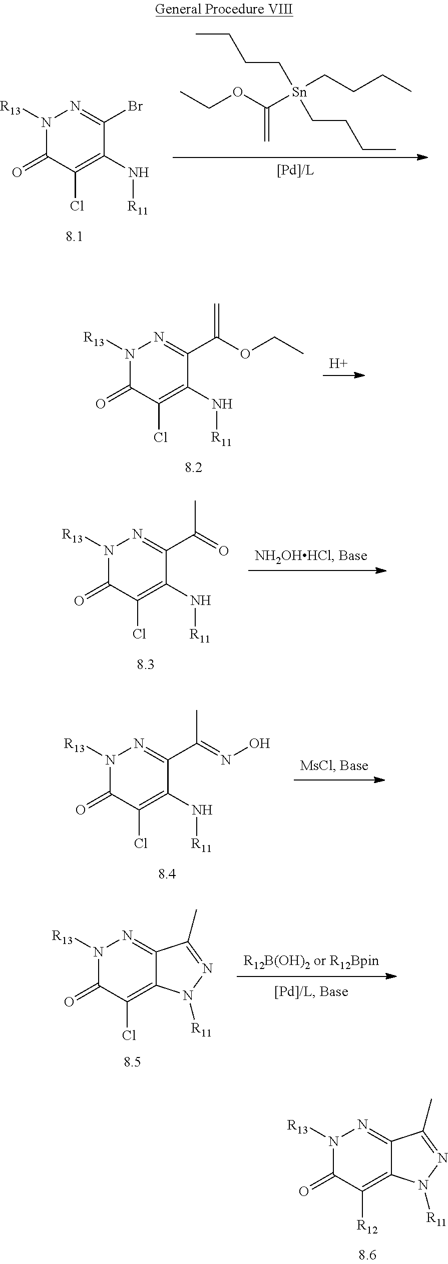

Compounds of structure 8.6 were obtained through the scheme depicted as General Procedure VIII. Beginning with intermediate 8.1, vinyl-ether 8.2 was obtained through a Stille cross-coupling with tributyl(1-ethoxyvinyl)tin. Compound 8.2 was subjected to acidic conditions to reveal ketone 8.3, which was then subjected to hydroxylamine and base to generate oxime 8.4. Compound 8.4 was reacted with MsCl and base to cyclize the pyrazole ring system and generate compound 8.5. The desired R₁₂ group was installed using a Suzuki coupling to afford final compounds of structure 8.6.

In some embodiments, the compounds of the present application, or pharmaceutically acceptable salts thereof, may be prepared by the general procedure IX.

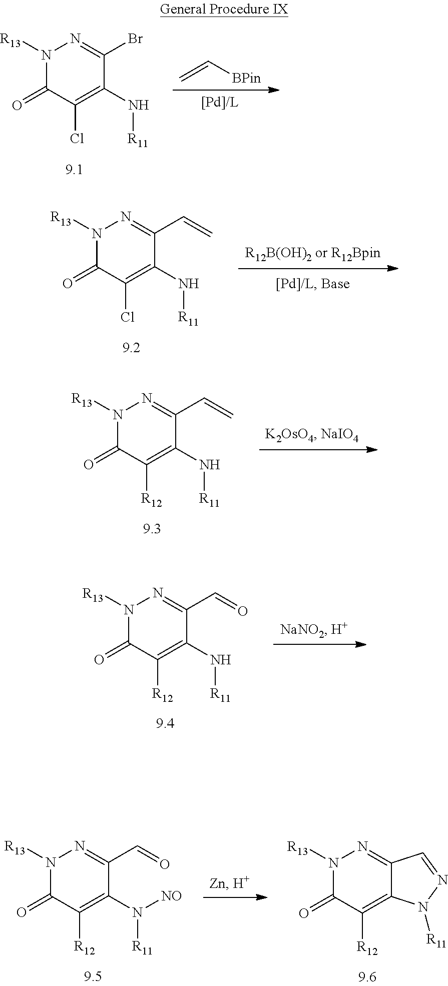

Compounds of structure 9.6 were obtained through the scheme depicted as General Procedure IX. Beginning with intermediate 9.1, compound 9.2 was obtained through a Suzuki cross-coupling with vinylboronic acid pinacol ester. The desired R₁₂ group was installed using a Suzuki cross-coupling to generate compound 9.3. Compound 9.3 was oxidatively cleaved using osmium tetroxide and sodium periodate to generate aldehyde 9.4. Heterocycle 9.4 was further subjected to sodium nitrite to generate nitroso compound 9.5, which was then subjected to reductive cyclization conditions to afford final compounds of structure 9.6.

In some embodiments, the compounds of the present application, or pharmaceutically acceptable salts thereof, may be prepared by the general procedure X.

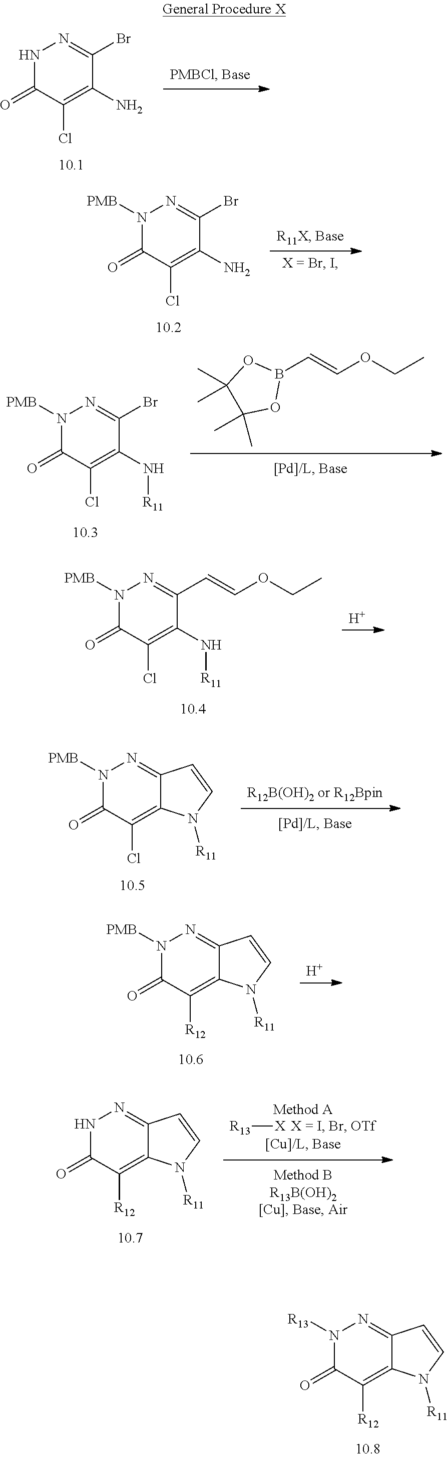

Compounds of structure 10.8 were obtained through the scheme depicted as General Procedure X. Pyridazinone 10.1 was PMB-protected through an N-alkylation reaction to generate compound 10.2. The desired Ru group was installed through an N-alkylation reaction to generate amino-heterocycle 10.3. Ethyl vinyl ether was then introduced through a Suzuki cross-coupling to generate intermediate 10.4, which was cyclized to bicyclic compound 10.5 upon exposure to acid. The desired R₁₂ group was introduced through a Suzuki cross-coupling reaction to generate compound 10.6, and the PMB protecting group was removed under acidic conditions to generate compound 10.7. The desired R₁₃ group was installed either through an Ullmann coupling (Method A) or a Chan-Lam coupling (Method B) to afford final compounds of structure 10.8.

### Methods of Use

The compounds described herein, or pharmaceutically acceptable salts thereof, may be used for inhibiting the synthesis of S-adenosyl methionine (SAM) in a subject in need thereof, the use comprising administering to the subject a SAM-synthesis inhibiting amount of a compound described herein, or a pharmaceutically acceptable salt thereof. In some aspects, the application provides the compounds disclosed herein, or pharmaceutically acceptable salts thereof, for treating a disease or condition in a subject suffering therefrom, wherein the disease or condition is responsive to a reduction in the concentration of SAM. In other embodiments, the compounds disclosed herein, or pharmaceutically acceptable salts thereof, treat a disease or condition in a subject by reducing the plasma concentration of SAM in a subject as compared to a control. In some embodiments the control is the plasma level of SAM in the subject prior to treatment with a compound of the application, or a pharmaceutically acceptable salt thereof.

In some aspects, a reduction in the plasma concentration of SAM results in a therapeutic benefit in a patient. The therapeutic benefit includes, without limitation, ameliorating a disease or disorder defined herein (i.e., arresting or reducing the development of the disease/disorder or at least one of the clinical symptoms thereof). In some embodiments, the therapeutic benefit includes modulating the disease or disorder. In further embodiments, the therapeutic benefit includes physically modulating the disease or disorder, i.e., stabilizing a discernible symptom. In yet other embodiments, the therapeutic benefit includes physiologically modulating the disease or disorder, i.e., stabilizing a physical parameter. In still further embodiments, the therapeutic benefit includes physically and physiologically modulating the disease or disorder. In other embodiments, the therapeutic benefit includes delaying the onset of the disease or disorder.

The term “SAM-synthesis inhibiting amount” as used herein refers to an amount of the compound of the application, or pharmaceutically acceptable salts thereof, that results in inhibition of SAM synthesis, which is reflected in the plasma concentration of SAM, as compared to a control. In some embodiments the control is the plasma level of SAM in the subject prior to treatment with a compound of the application, or a pharmaceutically acceptable salt thereof. In some embodiments, the SAM-synthesis inhibiting amount results in a reduction in the plasma concentration of SAM by at least 1%, as compared to a control. In some embodiments, the plasma concentration of SAM is reduced by at least about 5%, at least about 10%, at least about 20%, at least about 25%, at least about 30%, at least about 40%, at least about 50%, at least about 60%, at least about 70%, at least about 80%, at least about 90%, at least about 95%, at least about 99%, or about 100% as compared to a control. In other embodiments, inhibition of SAM synthesis is about 1% to about 100%, such as about 10% to about 100%, about 1% to about 90%, about 1% to about 80%, about 20% to about 100%, about 20% to about 90%, about 20% to about 80%, about 30% to about 100%, about 30% to about 90%, about 30% to about 80%, about 40% to about 100%, about 40% to about 90%, about 40% to about 80%, about 50% to about 100%, about 50% to about 90%, about 50% to about 80%, about 60% to about 100%, about 60% to about 90%, about 60% to about 80%, about 70% to about 100%, about 70% to about 90%, about 70% to about 80%, about 80% to about 100%, about 80% to about 90%, about 90% to about 100%, or about 95% to about 100% as compared to a control.

“Respond to the inhibition of MAT2A” or variations thereof as used herein refers to a disease or disorder that is ameliorated (i.e., arrested or a reduction in the development of the disease/disorder or at least one of the clinical symptoms thereof) due to inhibition of MAT2A using any of the compounds disclosed herein, or pharmaceutically acceptable salts thereof.

The compounds herein, or pharmaceutically acceptable salts thereof, are also useful in treating diseases or conditions that respond to the inhibition of MAT2A. In certain embodiments, the compounds herein, or pharmaceutically acceptable salts thereof, treat diseases or conditions in a subject suffering therefrom, wherein the disease or condition is responsive to the inhibition of MAT2A. In some embodiments, overexpression of MAT2A mediates certain cancers. Thus, the compounds of the application, or pharmaceutically acceptable salts thereof, may be used to treat such cancers. In certain embodiments, the cancer is neuroblastoma, rectum carcinoma, colon carcinoma, familiary adenomatous polyposis carcinoma and hereditary non-polyposis colorectal cancer, esophageal carcinoma, labial carcinoma, larynx carcinoma, hypopharynx carcinoma, tongue carcinoma, salivary gland carcinoma, gastric carcinoma, adenocarcinoma, medullary thyroidea carcinoma, papillary thyroidea carcinoma, renal carcinoma, kidney parenchym carcinoma, ovarian carcinoma, cervix carcinoma, uterine corpus carcinoma, endometrium carcinoma, chorion carcinoma, pancreatic carcinoma, prostate carcinoma, testis carcinoma, breast carcinoma, urinary carcinoma, melanoma, brain tumors such as glioblastoma, astrocytoma, meningioma, medulloblastoma and peripheral neuroectodermal tumors, Hodgkin lymphoma, non-Hodgkin lymphoma, Burkitt lymphoma, acute lymphatic leukemia (ALL), chronic lymphatic leukemia (CLL), acute myeloid leukemia (AML), chronic myeloid leukemia (CML), adult T-cell leukemia, lymphoma, hepatocellular carcinoma, gall bladder carcinoma, bronchial carcinoma, small cell lung carcinoma, non-small cell lung carcinoma, multiple myeloma, basalioma, teratoma, retinoblastoma, choroidea melanoma, seminoma, rhabdomyo sarcoma, craniopharyngioma, osteosarcoma, chondrosarcoma, myosarcoma, liposarcoma, fibrosarcoma, Ewing sarcoma, or plasmocytoma.

In certain embodiments, the application provides methods for treating a cancer in a subject suffering therefrom, wherein the cancer is characterized by a reduction or absence of methylthioadenosine phosphorylase (MTAP) gene expression, the absence of the MTAP gene, or reduced function of MTAP protein, the method comprising administering to the subject a therapeutically effective amount of a compound disclosed herein, or a pharmaceutically acceptable salt thereof. In further embodiments, the application provides compounds disclosed herein, or a pharmaceutically acceptable salt thereof, for treating a disease or condition in a subject suffering therefrom, wherein the disease or condition responds to the inhibition of MAT2A. In other embodiments, the application provides compounds disclosed herein, or pharmaceutically acceptable salts thereof, for treating a cancer in a subject suffering therefrom, wherein the cancer is characterized by a reduction or absence of methylthioadenosine phosphorylase (MTAP) expression, the absence of the MTAP gene, or reduced function of MTAP protein.

The term “suffering therefrom” or variations thereof as used herein refers to a subject that has, or is believed to have, a particular disease or condition. In some embodiments, the patient has been diagnosed with cancer. In other embodiments, the patient is suffering from or has been diagnosed with a disease or condition that is responsive to MAT2A inhibition. In still other embodiments, the patient is suffering from or has been diagnosed with a disease or condition that is responsive to a reduction in the plasma concentration of SAM.

In other embodiments, the application provides methods for treating an MTAP null cancer. In some embodiments, the cancer is characterized by a reduction or absence of MTAP expression, absence of the MTAP gene, or reduced function of MTAP protein as compared to cancers where the MTAP gene is present and fully functioning.

The terms “MTAP null,” “MTAP deleted,” “MTAP deficient,” and “absence of the MTAP gene” are used interchangeably and refer to cells in which the MTAP gene is absent, i.e., deleted or lost, deactivated, or has a reduced or impaired function. In some embodiments, a cancer is MTAP null.

In further embodiments, the application provides methods for treating an MTAP wild type (WT) cancer.

In some aspects, the present application provides methods for treating cancer in a subject wherein said cancer is characterized by reduction or absence of MTAP expression, absence of the MTAP gene, or reduced function of MTAP protein, and the cancer is further characterized by the presence of mutant KRAS and/or mutant p53. In some embodiments, the cancer is MTAP null and mutant KRAS. In other embodiments, the cancer is MTAP null and mutant p53. In further embodiments, the cancer is MTAP null and KRAS mutant. In yet other embodiments, the cancer is MTAP null, KRAS mutant and p53 mutant.

The term “mutant KRAS” or “KRAS mutation” refers to KRAS protein incorporating an activating mutation that alters its normal function and the gene encoding such a protein. For example, a mutant KRAS protein may incorporate a single amino acid substitution at position 12 or 13, such at position 12, or such as position 13, or such as positions 12 and 13. In certain embodiments, the KRAS mutant incorporates a G12X or G13X substitution, wherein X represents any amino acid change at the indicated position. In other embodiments, the KRAS mutant incorporates a G12X substitution. In further embodiments, the KRAS mutant incorporates a G13X substitution. In yet other embodiments, the substitution is G12V, G12R, G12C or G13D, such as G12V, or such as G12R, or such as G12C, or such as G13D.

By “mutant p53” or “p53 mutation” is meant p53 protein (or gene encoding said protein) incorporating a mutation that inhibits or eliminates its tumor suppressor function. In some embodiments, a p53 mutation is Y126_splice, K132Q, M133K, R174fs, R175H, R196*, C238S, C242Y, G245S, R248W, R248Q, I255T, D259V, S261_splice, R267P, R273C, R282W, A159V or R280K. In other embodiments, the mutant p53 cancer is non-small cell lung cancer (NSLCC), pancreatic cancer, head and neck cancer, gastric cancer, breast cancer, colon cancer or ovarian cancer.

In other embodiments, the compounds described herein, or pharmaceutically acceptable salts thereof, may be used to identify other compounds that are inhibitors of MAT2A, for example, in a competition assay for binding to MAT2A or for the inhibition of SAM production. Binding to MAT2A or the inhibition of SAM production by a test compound having a detectable label can be measured with and without the presence of an unlabeled compound of the present application, or a pharmaceutically acceptable salt thereof.

“Inhibitor” as used herein refers to a compound which prevents or reduces the amount of synthesis of S-adenosylmethionine (SAM) from methionine and ATP by MAT2A. In some embodiments, an inhibitor binds to MAT2A.

The compounds, or pharmaceutically acceptable salts thereof, may be administered prior to, concomitantly with, or following administration of radiation therapy or cytostatic or antineoplastic chemotherapy. In some embodiments, the compounds, or pharmaceutically acceptable salts thereof, are administered prior to, concomitantly with, or following administration of cytostatic or antineoplastic chemotherapy. Suitable cytostatic chemotherapy compounds include, but are not limited to (i) antimetabolites, such as cytarabine, fludarabine, 5-fluoro-2′-deoxyuiridine, gemcitabine, hydroxyurea or methotrexate; (ii) DNA-fragmenting agents, such as bleomycin, (iii) DNA-crosslinking agents, such as chlorambucil, cisplatin, cyclophosphamide or nitrogen mustard; (iv) intercalating agents such as adriamycin (doxorubicin) or mitoxantrone; (v) protein synthesis inhibitors, such as L-asparaginase, cycloheximide, puromycin or diphtheria toxin; (vi) topoisomerase I poisons, such as camptothecin or topotecan; (vii) topoisomerase II poisons, such as etoposide (VP-16) or teniposide; (viii) microtubule-directed agents, such as colcemid, colchicine, paclitaxel, vinblastine or vincristine; (ix) kinase inhibitors such as flavopiridol, staurosporine, STI571 (CPG 57148B) or UCN-01 (7-hydroxystaurosporine); (x) miscellaneous investigational agents such as thioplatin, PS-341, phenylbutyrate, ET-18—OCH₃, or farnesyl transferase inhibitors (L-739749, L-744832); polyphenols such as quercetin, resveratrol, piceatannol, epigallocatechin gallate, theaflavins, flavanols, procyanidins, betulinic acid and derivatives thereof; (xi) hormones such as glucocorticoids or fenretinide; (xii) hormone antagonists, such as tamoxifen, finasteride or LHRH antagonists. In some embodiments, the cytostatic compound is cisplatin, doxorubicin, taxol, taxotere or mitomycin C. In other embodiments, the cytostatic compound is doxorubicin.

The compounds described herein, or pharmaceutically acceptable salts thereof, may be used alone as an immuno-oncology therapy or in combination with an immuno-oncology therapy. In some embodiments, the compounds disclosed herein, or pharmaceutically acceptable salts thereof, are administered prior to, concomitantly with, or following administration of an immune checkpoint inhibitor. In certain embodiments, the checkpoint inhibitor is a PD-1 inhibitor, such as a PD-L1 inhibitor. In some embodiments, the checkpoint inhibitor is ipilimumab. In other embodiments, the checkpoint inhibitor is pembrolizumab, nivolumab, or atezolizumab.

The compounds disclosed herein, or pharmaceutically acceptable salts thereof, can be also used in combination with radiation therapy. The phrase radiation therapy” refers to electromagnetic or particulate radiation. Radiation therapy uses high-dose radiation delivered to a target area, resulting in the death of reproducing cells in both tumor and normal tissues. The radiation dosage regimen is generally defined in terms of radiation absorbed dose (rad), time and fractionation, and is determined by the oncologist. The amount of radiation depends on various consideration. In some embodiments, the amount of radiation depends on the location of the tumor in relation to other critical structures or organs of the body. In other embodiments, the amount of radiation depends on the extent to which the tumor has spread.

Examples of radiotherapeutic agents are provided in, but not limited to, radiation therapies known in the art (Hellman, Principles of Radiation Therapy, Cancer, in Principles I and Practice of Oncology, 24875 (Devita et al., 4th ed., vol 1, 1993, which is incorporated by reference herein). In some embodiments, radiation therapy includes three-dimensional conformal external beam radiation, intensity modulated radiation therapy (IMRT), stereotactic radiosurgery, or brachytherapy (interstitial radiation therapy), the latter placing the source of radiation directly into the tumor as implanted “seeds”. In other embodiments, the radiation therapy is three-dimensional. In other embodiments, the radiation therapy is conformal external beam radiation. In further embodiments, the radiation therapy is IMRT. In still other embodiments, the radiation therapy is stereotactic radiosurgery. In yet further embodiments, the radiation therapy is brachytherapy. In other embodiments, the radiation therapy is ionizing radiation with beta-emitting radionuclides. Depending on the tumor to be treated all kinds of emitters are conceivable within the scope of the present application. In some embodiments, gamma rays are used and deliver dosage at lower levels over much greater distances. In other embodiments, alpha particles are utilized and deliver very high LET dosage. Furthermore, the present application encompasses types of non-ionizing radiation like e.g. ultraviolet (UV) radiation, high energy visible light, microwave radiation (hyperthermia therapy), infrared (IR) radiation and lasers. In certain embodiments, UV radiation is applied.

### Pharmaceutical Compositions

The present application also includes pharmaceutical compositions or medicaments containing the compounds of the present application, or pharmaceutically acceptable salts thereof, and a pharmaceutically acceptable carrier, as well as methods of using the compounds of the present application, or pharmaceutically acceptable salts thereof, to prepare such compositions and medicaments. Typically, the compounds described herein, or pharmaceutically acceptable salts thereof, used in the methods of the present application are formulated by mixing at the appropriate pH, and at the desired degree of purity, with physiologically acceptable carriers, i.e., carriers that are non-toxic to recipients at the dosages and concentrations employed into a galenical administration form. The pH of the formulation depends mainly on the particular use and the concentration of compound, or a pharmaceutically acceptable salt thereof, and it can range from about 3 to about 8. In some embodiments, the formulation sterile. The compound, or a pharmaceutically acceptable salt thereof, ordinarily will be stored as a solid composition, although lyophilized formulations or aqueous solutions are acceptable.

In certain embodiments, pharmaceutical compositions of the present application may comprise a compound described herein, or a pharmaceutically acceptable salt thereof, and one or more polymer(s) as part of a solid dispersion (e.g., an amorphous solid dispersion). In other embodiments, the solid dispersion further comprises one or more surfactants. In further embodiments, the pharmaceutical composition comprising a compound of the present application, or a pharmaceutically acceptable salt thereof, is a solid spray-dried dispersion. Pharmaceutical compositions comprising solid dispersions of a compound of the present application, or a pharmaceutically acceptable salt thereof, in a matrix may provide improved chemical and physical properties and can be prepared by forming a homogeneous solution or melt of the compound of the present application, or a pharmaceutically acceptable salt thereof, and matrix material followed by solidifying the mixture by cooling, or removal of the solvent. Such solid dispersions may show enhanced bioavailability when administered orally relative to oral compositions comprising the undispersed compound, or a pharmaceutically acceptable salt thereof.

The term “dispersion” as used herein refers to a disperse system in which one substance, the dispersed phase, is distributed, in discrete units, throughout a second substance (the continuous phase or vehicle). The size of the dispersed phase can vary considerably (e.g., colloidal particles of nanometer dimension, to multiple microns in size). In general, the dispersed phases can be solids, liquids, or gases. In the case of a solid dispersion, the dispersed and continuous phases are both solids. In pharmaceutical applications, a solid dispersion can include a crystalline therapeutically active compound (dispersed phase) in an amorphous polymer(s) (continuous phase), or alternatively, an amorphous therapeutically active compound (dispersed phase) in an amorphous polymer (continuous phase). An amorphous solid dispersion generally refers to a solid dispersion of two or more components, such as a compound of the present application, or a pharmaceutically acceptable salt thereof, and polymer (or plurality of polymers), but possibly containing other components such as surfactants or other pharmaceutical excipients, where the compound of the present application, or a pharmaceutically acceptable salt thereof, is in the amorphous phase. In some embodiments, an amorphous solid dispersion includes the polymer(s) (and optionally a surfactant) constituting the dispersed phase, and the compound of the present application, or a pharmaceutically acceptable salt thereof, constitutes the continuous phase. In other embodiments, an amorphous solid dispersion includes the polymer(s) (and optionally a surfactant) constituting the continuous phase, and the compound of the present application, or a pharmaceutically acceptable salt thereof, constitutes the dispersed phase.

An exemplary solid dispersion is a co-precipitate or a co-melt of a compound of the present application, or a pharmaceutically acceptable salt thereof, with one or more polymer(s). A “co-precipitate” is produced after dissolving a compound of the present application, or a pharmaceutically acceptable salt thereof, and one or more polymers in a solvent or solvent mixture followed by the removal of the solvent or solvent mixture. In some embodiments, the one or more polymers is suspended in the solvent or solvent mixture. The solvent or solvent mixture includes organic solvents and supercritical fluids. The solvent or solvent mixture can also contain a non-volatile solvent. A “co-melt” is produced after heating a compound of the present application, or a pharmaceutically acceptable salt thereof, and one or more polymers to melt, optionally in the presence of a solvent or solvent mixture, followed by mixing, removal of at least a portion of the solvent if applicable, and cooling to room temperature at a selected rate. In some embodiments, solid dispersions are prepared by adding a solution of a therapeutically active compound and solid polymers followed by mixing and removal of the solvent or solvent mixture. To remove the solvent or solvent mixture, vacuum drying, spray drying, tray drying, lyophilization, and other drying procedures may be applied. Applying any of these methods using appropriate processing parameters, according to this application, would provide the particular therapeutically active compound in an amorphous state in the final solid dispersion product.

The composition of the present application will be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

The “effective amount” of the compound to be administered will be governed by such considerations, and is the minimum amount necessary to inhibit MAT2A activity and/or the minimum amount necessary to reduce the plasma concentration of SAM. Such amount may be below the amount that is toxic to normal cells, or the mammal as a whole. Generally, the initial pharmaceutically effective amount of the compound of the present application, or a pharmaceutically acceptable salt thereof, administered per dose will be in the range of about 0.01 to about 2000 mg/kg. In some embodiments, the effective amount is about 0.01 to about 200 mg/kg, about 0.1 to about 150 mg/kg, about 0.1 to about 100 mg/kg, about 0.1 to about 75 mg/kg, about 0.1 to about 50 mg/kg, about 0.1 to about 25 mg/kg, about 0.1 to 20 mg/kg, about 0.1 to about 15 mg/kg, about 0.1 to about 10 mg/kg, about 0.1 to about 5 mg/kg, about 0.1 to about 1 mg/kg, about 0.3 to about 150 mg/kg, about 0.3 to about 100 mg/kg, about 0.3 to about 75 mg/kg, about 0.3 to about 50 mg/kg, about 0.3 to about 25 mg/kg, about 0.3 to 20 mg/kg, about 0.3 to about 15 mg/kg, about 0.3 to about 10 mg/kg, about 0.3 to about 5 mg/kg, about 0.3 to about 1 mg/kg of patient body weight per day. In other embodiments, the effective amount is about 0.01 mg/kg, about 0.1 mg/kg, about 0.3 mg/kg, about 0.5 mg/kg, about 1 mg/kg, about 5 mg/kg, about 10 mg/kg, about 25 mg/kg, about 50 mg/kg, about 75 mg/kg, about 100 mg/kg, about 200 mg/kg, about 300 mg/kg, about 400 mg/kg, about 500 mg/kg, about 600 mg/kg, about 700 mg/kg, about 800 mg/kg, about 900 mg/kg, about 1000 mg/kg, about 1100, about 1200 mg/kg, about 1300 mg/kg, about 1400 mg/kg, about 1500 mg/kg, about 1600, about 1700, about 1800, about 1900 mg/kg, or about 2000 mg/kg. In further embodiments, the effective amount is about 0.01 to about 2000 mg. In some embodiments, the effective amount is about 0.01 to about 1900 mg, about 0.01 to about 1800 mg, about 0.01 to about 1700 mg, about 0.01 to about 1600 mg, about 0.01 to about 1500 mg, about 0.01 to about 1400 mg, 0.01 to about 1200 mg, about 0.01 to about 1100 mg, about 0.01 to about 1000 mg, about 0.01 to about 900 mg, 0.01 to about 800 mg, about 0.01 to about 700 mg, about 0.01 to about 600 mg, about 0.01 to about 500 mg, about 0.01 to about 400 mg, 0.01 to about 300 mg, about 0.01 to about 200 mg, about 0.1 to about 150 mg, about 0.1 to about 100 mg, about 0.1 to about 75 mg, about 0.1 to about 50 mg, about 0.1 to about 25 mg, about 0.1 to 20 mg, about 0.1 to about 15 mg, about 0.1 to about 10 mg, about 0.1 to about 5 mg, about 0.1 to about 1 mg, about 0.3 to about 150 mg, about 0.3 to about 100 mg, about 0.3 to about 75 mg, about 0.3 to about 50 mg, about 0.3 to about 25 mg, about 0.3 to 20 mg, about 0.3 to about 15 mg, about 0.3 to about 10 mg, about 0.3 to about 5 mg, about 0.3 to about 1 mg. In other embodiments, the effective amount is about 0.01 mg, about 0.1 mg, about 0.3 mg, about 0.5 mg, about 1 mg, about 5 mg, about 10 mg, about 15 mg, about 25 mg, about 50 mg, about 75 mg, about 100 mg, about 200 mg, about 250 mg, about 300 mg, about 400 mg, about 500 mg, about 600 mg, about 700 mg, about 750 mg, about 800 mg, about 900 mg, about 1000 mg, about 1100, about 1200 mg, about 1250 mg, about 1300 mg, about 1400 mg, about 1500 mg, about 1600, about 1700, about 1750 mg, about 1800, about 1900 mg, or about 2000 mg.

The compound of the present application, or a pharmaceutically acceptable salt thereof, may be administered by any suitable means, including oral, topical, transdermal, parenteral, subcutaneous, intraperitoneal, intrapulmonary, intranasal, or intralesional administration, e.g., for local treatment. Parenteral infusions include intramuscular, intravenous, intraarterial, intraperitoneal, or subcutaneous administration. In some embodiments, administration is oral. In other embodiments, administration is topical. In further embodiments, administration is transdermal. In yet other embodiments, administration is parenteral. In still further embodiments, administration is subcutaneous. In other embodiments, administration is intraperitoneal. In further embodiments, administration is intrapulmonary. In still other embodiments, administration is intranasal. In yet further embodiments, administration is intralesional. In other embodiments, administration is intramuscular. In further embodiments, administration is intravenous. In still other embodiments, administration is intraarterial. In yet further embodiments, administration is intraperitoneal.

### Aspects

Aspect 1: A compound of Formula I, or a pharmaceutically acceptable salt thereof:

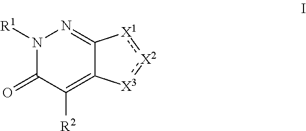

wherein:
X¹ is N, NR⁶, CR⁴, or CR⁴R^{4′};
X² is N, CR⁵, or CR⁵R⁵;
X³ is N, NR³, or CR³;
X¹ [symbol] X² and X² [symbol] X³ are, independently, a double or single bond, provided that (i) both X¹ [symbol] X² and X² [symbol] X³ are not double bonds and (i) both X¹ [symbol] X² and X² [symbol] X³ are single bonds when X² is C(O);
R¹ is C₆₋₁₄aryl or 3- to 14-membered heteroaryl, wherein each is optionally substituted, as permitted, by one or more of R^{A};
R² is —C₀₋₆alkyl-C₆₋₁₄aryl or —C₀₋₆alkyl-(3- to 14-membered heteroaryl), wherein each, as permitted, is optionally substituted by one or more of R^{A};
R³ is H, D, CN, halo, OH, C₁₋₆alkyl, C₁₋₆alkylOH, deuterated C₁₋₆alkyl, C₂₋₆alkenyl, C₂₋₆haloalkenyl, C₂₋₆alkynyl, C₁₋₆alkoxy, deuterated C₁₋₆alkoxy, C₁₋₆haloalkoxy, C₁₋₆haloalkyl, —C₀₋₆alkyl-C₃₋₁₂carbocyclyl, —C₀₋₆alkyl-C₀₋₆alkyl-O—C_{6 14}aryl, —C₀₋₆alkyl-(3- to 14-membered heterocyclyl), —C₀₋₆alkyl-O-(3- to 14-membered heterocyclyl), —C₀₋₆alkyl-(3- to 14-membered heteroaryl), —C₀₋₆alkyl-NR^{A}(CH₂)₀₋₆C(O)R^{A} NR^{B}R^{C}, C(O)NR^{B}R^{C}, NR^{B}C(NR^{C})NR^{B}R^{C}, NR^{B}C(NR^{C})(NR^{B}), —S(O)₀₋₂(C₁₋₆alkyl), —S(O)₀₋₂(C₁₋₆alkyl)₂, —S(O)₀₋₂NH₂, —S(O)₀₋₂NHC₁₋₆alkyl, —S(O)₀₋₂(C₆₋₁₄aryl), —S(O)₀₋₂(heteroaryl), —S(O)₀₋₂(heterocyclyl), —C(O)(C₁₋₆alkyl), —C(O)(C₃₋₁₄carbocyclyl), —C(O)NR^{A}R^{B}, —C(O)(C₆₋₁₄aryl), —C(O)(heteroaryl), —C(O)(heterocyclyl), or SR^{B} wherein each of the forgoing, as permitted, is optionally substituted by one or more of R^{A};
R⁴, R^{4′}, R⁵, and R^{5′} are, independently, H, D, CN, OH, C₁₋₆alkyl, halo, deuterated C₁₋₆alkyl, C₁₋₆alkoxy, deuterated C₁₋₆alkoxy, C₁₋₆alkylOH, C₁₋₆haloalkyl, C₁₋₆haloalkoxy, C₂₋₆alkenyl, C₂₋₆haloalkenyl, C₂₋₆alkynyl, —S(O)₀₋₂NH₂, —S(O)₀₋₂NHC₁₋₆alkyl, —S(O)₀₋₂(C₁₋₆alkyl)₂, —S(O)₀₋₂C₁₋₆alkyl, —S(O)₀₋₂(C₆₋₁₄aryl), —S(O)₀₋₂(heteroaryl), —S(O)₀₋₂(heterocyclyl), —C₀₋₆alkyl-(C₃₋₁₄carbocyclyl), —C₀₋₆alkyl-C₆₋₁₄aryl, —C₀₋₆alkyl-O—C₆₋₁₄aryl, C₀₋₆alkyl-(3- to 14-membered heterocyclyl), —C₀₋₆alkyl-O-(3- to 14-membered heterocyclyl), C₀₋₆alkyl-(3- to 14-membered heteroaryl), —C₁₋₆alkyl-NR^{A}(CH₂)₀₋₆C(O)R^{A}, NR^{B}R^{C}, C(O)NR^{B}R^{C}, NR^{B}C(NR^{C})NR^{B}R^{C}, NR^{B}C(NR^{C})(═NR^{B}), —C(O)NR^{A}R^{B}, —C(O)(C₁₋₆alkyl), —C(O)(C₃₋₁₄carbocyclyl), —C(O)(heteroaryl), —C(O)(heterocyclyl), —C(O)(C₆₋₁₄aryl), or SR^{B} wherein each of the forgoing, as permitted, is optionally substituted by one or more R^{A};
or R⁴ and R^{4′} or R⁵ and R^{5′} are combined to form oxo; and
R⁶ is H or C₁₋₆alkyl;
R^{A} is D, CN, NH₂, halo, C₁₋₆alkyl, deuterated C₁₋₆alkyl, C₁₋₆haloalkyl, C₁₋₆alkoxy, C₁₋₆haloalkoxy, deuterated C₁₋₆alkoxy, OH, —C₁₋₆alkylOH, C₂₋₆alkenyl, C₂₋₆haloalkenyl, C₂₋₆alkynyl, —S(O)₀₋₂(C₁₋₆alkyl), —S(O)₀₋₂(C₆₋₁₄aryl), —S(O)₀₋₂NH₂, —S(O)₀₋₂NHC₁₋₆alkyl, —S(O)₀₋₂(C₁₋₆alkyl)₂, —S(O)₀₋₂(heteroaryl), —S(O)₀₋₂(heterocyclyl), —C(O)(C₁₋₆alkyl), —C(O)NH₂, —C(O)NHC₁₋₆alkyl, —C(O)NH(C₁₋₆alkyl)₂, —C(O)(C₃₋₁₄carbocyclyl), —C(O)(heteroaryl), —C(O)(heterocyclyl), —C(O)(C₆₋₁₄aryl), —C₀₋₆alkyl-C₃₋₁₄carbocyclyl, —C₀₋₆alkyl-C₆₋₁₄aryl, —C₀₋₆alkyl-O—C₆₋₁₄aryl, —C₀₋₆alkyl-(3- to 14-membered heterocyclyl), —C₀₋₆alkyl-(3- to 14-membered heteroaryl), or —C₀₋₆alkyl-O-(3- to 14-membered heterocyclyl),
   wherein R^{A}, as permitted, is optionally substituted by D, CN, halo, C₁₋₆alkyl, deuterated C₁₋₆alkyl, C₁₋₆haloalkyl, —C₀₋₆alkyl-C₃₋₁₄carbocyclyl, C₁₋₆alkoxy, C₁₋₆haloalkoxy, deuterated C₁₋₆alkoxy, OH, —C₁₋₆alkylOH, C₂₋₆alkenyl, C₂₋₆haloalkenyl, C₂₋₆alkynyl, —S(O)₀₋₂(C₁₋₆alkyl), —S(O)₀₋₂(C₆₋₁₄aryl), —S(O)₀₋₂(heteroaryl), —S(O)₀₋₂(heterocyclyl), —S(O)₀₋₂NH₂, —S(O)₀₋₂NHC₁₋₆alkyl, —S(O)₀₋₂(C₁₋₆alkyl)₂, —C(O)(C₁₋₆alkyl), —C(O)NH₂, —C(O)NHC₁₋₆alkyl, —C(O)NH(C₁₋₆alkyl)₂, —C(O)(C₃₋₁₄carbocyclyl), —C(O)(C₆₋₁₄aryl), —C(O)(heteroaryl), —C(O)(heterocyclyl), —C₀₋₆alkyl-O—C₆₋₁₄aryl, —C₀₋₆alkyl-C₆₋₁₄aryl, —C₀₋₆alkyl-(3- to 14-membered heterocyclyl), —C₀₋₆alkyl-(3- to 14-membered heteroaryl), —C₀₋₆alkyl-O-(3- to 14-membered heterocyclyl), —C₀₋₆alkyl-NH₂, —C₀₋₆alkyl-NH(alkyl), or —C₀₋₆alkyl-N(alkyl)₂;
R^{B} and R^{C} are, independently, H, D, CN, NH₂, halo, OH, C₁₋₆alkyl, deuterated C₁₋₆alkyl, C₁₋₆haloalkyl, —C₀₋₆alkyl-C₃₋₁₄carbocyclyl, C₁₋₆alkoxy, C₁₋₆haloalkoxy, deuterated C₁₋₆alkoxy, —C₁₋₆alkylOH, C₂₋₆alkenyl, C₂₋₆haloalkenyl, C₂₋₆alkynyl, —S(O)₀₋₂(C₁₋₆alkyl), —S(O)₀₋₂NH₂, —S(O)₀₋₂NHC₁₋₆alkyl, —S(O)₀₋₂(C₁₋₆alkyl)₂, —S(O)₀₋₂(C₆₋₁₄aryl), —S(O)₀₋₂(heteroaryl), —S(O)₀₋₂(heterocyclyl), —C(O)(C₁₋₆alkyl), —C(O)NH₂, —C(O)NHC₁₋₆alkyl, —C(O)NH(C₁₋₆alkyl)₂, —C(O)(C₃₋₁₄carbocyclyl), —C(O)(heteroaryl), —C(O)(heterocyclyl), —C(O)(C₆₋₁₄aryl), —C₀₋₆alkyl-C₆₋₁₄aryl, —C₀₋₆alkyl-O—C₆₋₁₄aryl, —C₀₋₆alkyl-(3- to 14-membered heterocyclyl), —C₀₋₆alkyl-(3- to 14-membered heteroaryl), —C₀₋₆alkyl-O-(3- to 14-membered heterocyclyl), —C₀₋₆alkyl-NH₂, —C₀₋₆alkyl-NH(alkyl), or —C₀₋₆alkyl-N(alkyl)₂;
   wherein each of the forgoing, as permitted, is optionally substituted by D, CN, halo, OH, C₁₋₆alkyl, deuterated C₁₋₆alkyl, C₁₋₆haloalkyl, —C₀₋₆alkyl-C₃₋₁₄carbocyclyl, C₁₋₆alkoxy, C₁₋₆haloalkoxy, deuterated C₁₋₆alkoxy, —C₁₋₆alkylOH, C₂₋₆alkenyl, C₂₋₆haloalkenyl, C₂₋₆alkynyl, —S(O)₀₋₂(C₁₋₆alkyl), —S(O)₀₋₂(C₆₋₁₄aryl), —S(O)₀₋₂(heteroaryl), —S(O)₀₋₂(heterocyclyl), —S(O)₀₋₂NH₂, —S(O)₀₋₂NHC₁₋₆alkyl, —S(O)₀₋₂(C₁₋₆alkyl)₂, —C(O)(C₁₋₆alkyl), —C(O)NH₂, —C(O)NHC₁₋₆alkyl, —C(O)NH(C₁₋₆alkyl)₂, —C(O)(C₃₋₁₄carbocyclyl), —C(O)(C₆₋₁₄aryl), —C(O)(heteroaryl), —C(O)(heterocyclyl), —C₀₋₆alkyl-O—C₆₋₁₄aryl, —C₀₋₆alkyl-C₆₋₁₄aryl, —C₀₋₆alkyl-(3- to 14-membered heterocyclyl), —C₀₋₆alkyl-3- to 14-membered heteroaryl, or —C₀₋₆alkyl-O-(3- to 14-membered heterocyclyl), —C₀₋₆alkyl-NH₂, —C₀₋₆alkyl-NH(alkyl), or —C₀₋₆alkyl-N(alkyl)₂;
wherein each heteroaryl or heterocyclyl, independently, comprises 1 to 4 heteroatoms that are, independently, N, O, or S.

Aspect 2: The compound of Aspect 1, wherein R¹ is optionally substituted C₆₋₁₄-aryl, for example optionally substituted C₆₋₁₀aryl, or for example optionally substituted C₆₋₈aryl.

Aspect 3: The compound of Aspect 1 or 2, wherein R¹ is optionally substituted phenyl or optionally substituted naphthyl, for example optionally substituted phenyl.

Aspect 4: The compound of any one of the preceding Aspects, wherein R¹ is:

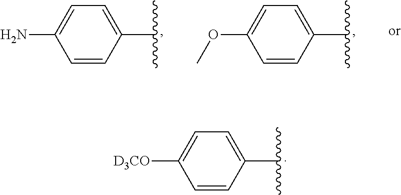

Aspect 5: The compound of Aspect 1, wherein R¹ is optionally substituted 3- to 14-membered heteroaryl, for example optionally substituted 5- to 12-membered heteroaryl, or for example optionally substituted 6- to 10-membered heteroaryl.

Aspect 6: The compound of Aspect 5, wherein the optionally substituted 3- to 14-membered heteroaryl comprises at least one N, or one N and one S, or two N, or one N and one O.

Aspect 7: The compound of Aspect 5, wherein the optionally substituted 3- to 14-membered heteroaryl is thienyl, furyl, imidazolyl, pyrazolyl, thiazolyl, isothiazolyl, oxazolyl, isoxazolyl, triazolyl, thiadiazolyl, oxadiazolyl, tetrazolyl, thiatriazolyl, oxatriazolyl, pyridyl, pyrimidyl, pyrazinyl, pyridazinyl, thiazinyl, oxazinyl, triazinyl, thiadiazinyl, oxadiazinyl, dithiazinyl, dioxazinyl, oxathiazinyl, tetrazinyl, thiatriazinyl, oxatriazinyl, dithiadiazinyl, imidazolinyl, dihydropyrimidyl, tetrahydropyrimidyl, tetrahydropyridinyl, tetrazolopyridazinyl, purinyl, benzoxazolyl benzoxazinyl, benzofuryl, benzothiazolyl, benzothiadiazolyl, benzotriazolyl, benzoimidazolyl, indolyl, or quinolinyl.

Aspect 8: The compound of Aspect 5 or 6, wherein the optionally substituted 3- to 14-membered heteroaryl is pyridyl, benzothiazolyl, quinolinyl, pyrazolyl, benzoxazinyl such as benzo[1,4]oxazinyl, tetrahydropyridinyl such as 1,2,3,6-tetrahydropyridin-yl, or benzimidazolyl such as benzo[d]imidazolyl.

Aspect 9: The compound of any one of Aspects 1 or 5 to 8, wherein R¹ is:

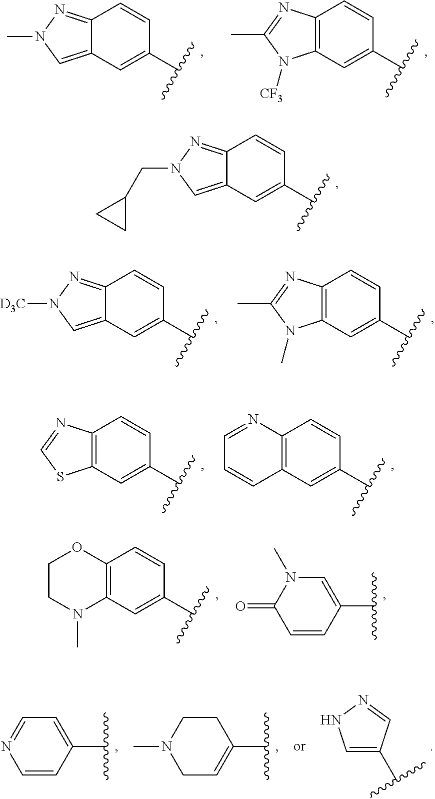

Aspect 10: The compound of any one of the preceding Aspects, wherein R¹ is substituted with one or more of:
C₁₋₆alkyl, such as methyl, ethyl, propyl, butyl, pentyl, or hexyl, for example methyl,
deuterated C₁₋₆alkyl, such as CH₂D, CHD₂, or CD₃, for example CD₃;
C₁₋₆haloalkyl, such as C₁₋₆fluoroalkyl, for example CF₃, CHF₂, CH₂F, or for example CF₃;
NH₂;
C₁₋₆alkoxy, such as methoxy, ethoxy, propoxy, butoxy, pentoxy, or hexoxy, for example methoxy;
—C₀₋₆alkyl-C₃₋₈carbocylyl, such as —C₀₋₆alkyl-C₃₋₈cycloalkyl or —CH₂—C₃₋₈cycloalkyl or C₀₋₆alkyl-cyclopropyl, for example, CH₂-cyclopropyl, CH₂-cyclobutyl, CH₂-cyclopentyl, CH₂-cyclohexyl, or for example CH₂-cyclopropyl, or for example CH₂CH₂-cyclopropyl.

Aspect 11: The compound of any one of the preceding Aspects, wherein R² is —C₀₋₆alkyl-optionally substituted C₆₋₁₄-aryl, for example —C₀₋₆alkyl-optionally substituted C₆₋₁₄-aryl, or for example optionally substituted C₆₋₁₀aryl, or for example optionally substituted C₈₋₁₀aryl.

Aspect 12: The compound of any one of the preceding Aspects, wherein R² is optionally substituted phenyl or naphthyl, for example optionally substituted phenyl.

Aspect 13: The compound of any one of the preceding Aspects, wherein R² is:

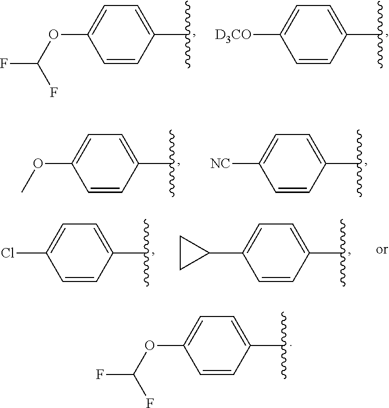

Aspect 15: The compound of any one of Aspects 1 to 10, wherein R² is —C₀₋₆alkyl-(optionally substituted 3- to 14-membered heteroaryl), for example optionally substituted 3- to 14-membered heteroaryl, or for example optionally substituted 5- to 12-membered heteroaryl, or for example optionally substituted 6- to 10-membered heteroaryl.

Aspect 15: The compound of Aspect 14, wherein the —C₀₋₆alkyl-optionally substituted heteroaryl comprises at least one N, or one N and one S, or two N, or one N and one O.

Aspect 16: The compound of Aspect 14, wherein the —C₀₋₆alkyl-optionally substituted heteroaryl is thienyl, furyl, imidazolyl, pyrazolyl, thiazolyl, isothiazolyl, oxazolyl, isoxazolyl, triazolyl, thiadiazolyl, oxadiazolyl, tetrazolyl, thiatriazolyl, oxatriazolyl, pyridyl, pyrimidyl, pyrazinyl, pyridazinyl, thiazinyl, oxazinyl, triazinyl, thiadiazinyl, oxadiazinyl, dithiazinyl, dioxazinyl, oxathiazinyl, tetrazinyl, thiatriazinyl, oxatriazinyl, dithiadiazinyl, imidazolinyl, dihydropyrimidyl, tetrahydropyrimidyl, tetrahydropyridinyl, tetrazolopyridazinyl, purinyl, benzoxazolyl benzoxazinyl, benzofuryl, benzothiazolyl, benzothiadiazolyl, benzotriazolyl, benzoimidazolyl, indolyl, quinolinyl.

Aspect 17: The compound of Aspect 14 or 15, wherein the —C₀₋₆alkyl-optionally substituted heteroaryl is pyridyl, benzothiazolyl, quinolinyl, pyrazolyl, benzoxazinyl such as benzo[1,4]oxazinyl, tetrahydropyridinyl such as 1,2,3,6-tetrahydropyridin-yl, or benzimidazolyl such as benzo[d]imidazolyl, for example pyridyl.

Aspect 18: The compound of any one of Aspects 1 to 10 or 14 to 17, wherein R² is:

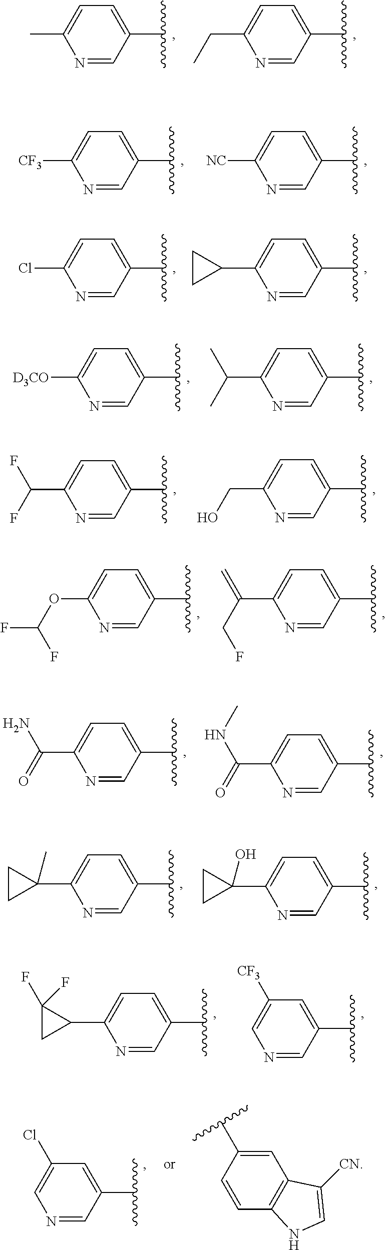

Aspect 19: The compound of any one of the preceding Aspects wherein R² is substituted with one or more of:
halo, such as F, Cl, Br, or I, for example F or Cl, or for example Cl;
CN;
C₁₋₆alkyl, such as methyl, ethyl, propyl, butyl, pentyl, or hexyl, or for example methyl;
deuterated C₁₋₆alkyl, such as CH₂D, CHD₂, or CD₃, for example CD₃;
C₁₋₆haloalkyl, such as C₁₋₆fluoroalkyl, for example CF₃, CHF₂, CH₂F, or for example CF₃;
C₁₋₆alkylOH, such as CH₂OH, CH₂CH₂OH, propyl-OH, butyl-OH, pentyl-OH, or hexyl-OH, for example CH₂OH;
C₁₋₆alkoxy such as methoxy, ethoxy, propoxy, butoxy, pentoxy, or hexoxy, for example methoxy;
C₁₋₆haloalkoxy, such as C₁₋₆fluoroalkoxy, for example OCF₃, OCHF₂, OCH₂F, or for example OCF₃;
deuterated C₁₋₆alkoxy, such as OCH₂D, OCHD₂, or OCD₃, for example OCD₃;
C₂₋₆haloalkenyl, such as C₂₋₆fluoroalkenyl, or for example fluoroethenyl, fluoropropenyl, fluorobutenyl, fluoropentenyl, or fluorohexenyl, or for example fluoropropenyl;
—C₀₋₆alkyl-C₃₋₁₀carbocyclyl, for example C₃₋₁₀carbocyclyl, such as C₃₋₁₀cycloalkyl or C₃₋₈cycloalkyl or C₃₋₆cycloalkyl, such as cyclopropyl, cyclobutyl, cyclopentyl, cyclohexyl, or for example cyclopropyl;
C(O)NH₂; or
C(O)NHC₁₋₆alkyl, such as C(O)NHmethyl, C(O)NHethyl, C(O)NHpropyl, C(O)NHbutyl, C(O)NHpentyl, or C(O)NHhexyl, for example C(O)NHmethyl.

Aspect 20: The compound of Aspect 19, wherein R² is substituted with cyclopropyl.

Aspect 21: The compound of Aspect 19 or 20, wherein the R² substituent is substituted with one or more of:
CN;
halo such as F, Cl, Br, or I, for example F or Cl, or for example Cl;
OH; or
C₁₋₆alkyl such as methyl, ethyl, propyl, butyl, pentyl, or hexyl, or for example methyl.

Aspect 22: The compound of any one of the preceding Aspects wherein X¹ [symbol] [symbol] X² and X² [symbol] X³ are single bonds.

Aspect 23: The compound of any one of Aspects 1 to 21, wherein X¹ [symbol] X² is a single bond and X¹ [symbol] X³ is a double bond.

Aspect 24: The compound of any one of Aspects 1 to 21, wherein X¹ [symbol] X² is a double bond and X² [symbol] X³ is a double bond.

Aspect 25: The compound of any one of Aspects 1 to 21 or 23, wherein X¹ is N.

Aspect 26: The compound of any one of Aspects 1 to 23, wherein X¹ is NR⁶.

Aspect 27: The compound of any one of the preceding Aspects, wherein R⁶ is H.

Aspect 28: The compound of any one of Aspects 1 to 26, wherein R⁶ is C₁₋₆alkyl, such as methyl, ethyl, propyl, butyl, pentyl, or hexyl, or for example methyl.

Aspect 29: The compound of any one of Aspects 1 to 21 or 24, wherein X¹ is CR⁴.

Aspect 30: The compound of any one of the preceding Aspects, wherein R⁴ is:
H;
CN;
C₁₋₆alkyl, such as methyl, ethyl, propyl, butyl, pentyl, or hexyl, or for example methyl, or for example ethyl, or for example propyl such as n-propyl or i-propyl;
halo, such as F, Cl, Br, or I, for example F, or for example Cl, or for example Br; or for example I;
C₁₋₆alkylOH, such as CH₂OH, CH₂CH₂OH, propyl-OH, butyl-OH, pentyl-OH, or hexyl-OH, or for example CH₂OH, or for example CH(OH)CH₃;
C₁₋₆haloalkyl, such as C₁₋₆fluoroalkyl, for example CF₃, CHF₂, CH₂F, or for example CF₃, or for example CHF₂;
C₂₋₆alkynyl, such as ethynyl, propynyl, butynyl, pentynyl, or hexynyl, or for example ethynyl;
C(O)OH;
C(O)C₁₋₆alkyl, such as C(O)methyl, C(O)ethyl, C(O)propyl, C(O)butyl, C(O)pentyl, or C(O)hexyl, or for example C(O)methyl;
C(O)NH₂;
C(O)NHC₁₋₆alkyl, such as C(O)NHmethyl, C(O)NHethyl, C(O)NHpropyl, C(O)NHbutyl, C(O)NHpentyl, or C(O)NHhexyl, or for example C(O)NHmethyl;
C(O)N(C₁₋₆alkyl)₂, such as C(O)N(methyl)₂, C(O)N(ethyl)₂, C(O)N(propyl)₂, C(O)N(butyl)₂, C(O)N(pentyl)₂, or C(O)N(hexyl)₂, or for example C(O)N(methyl)₂;
S(O)₀₋₂NH₂ for example S(O)₂NH₂;
S(O)₀₋₂NHC₁₋₆alkyl, for example S(O)₂NHC₁₋₆alkyl such as S(O)₂NHmethyl, S(O)₂NHethyl, S(O)₂NHpropyl, S(O)₂NHbutyl, S(O)₂NHpentyl, or S(O)₂NHhexyl, or for example S(O)₂NHmethyl;
S(O)₀₋₂N(C₁₋₆alkyl)₂, for example S(O)₂N(C₁₋₆alkyl)₂ such as S(O)₂N(methyl)₂, S(O)₂N(ethyl)₂, S(O)₂N(propyl)₂, S(O)₂N(butyl)₂, S(O)₂N(pentyl)₂, or S(O)₂N(hexyl)₂, or for example S(O)₂N(methyl)₂; or
S(O)₀₋₂C₁₋₆alkyl, for example S(O)₂C₁₋₆alkyl such as S(O)₂methyl, S(O)₂ethyl, S(O)₂propyl, S(O)₂butyl, S(O)₂pentyl, or S(O)₂hexyl, or for example S(O)₂methyl;

Aspect 31: The compound of Aspect 30, wherein R⁴ is C₂₋₆alkynyl.

Aspect 31: The compound of Aspect 31, wherein R⁴ is substituted by Si(alkyl)₃ such as Si(CH₃)₃ or C₁₋₆haloalkyl, such as C₁₋₆fluoroalkyl, for example CF₃, CHF₂, CH₂F, or for example CF₃, or for example CHF₂.

Aspect 33: The compound of Aspect 30, wherein R⁴ is C₁₋₆alkyl.

Aspect 34: The compound of Aspect 33, wherein R⁴ is substituted by CN or the C₁₋₆alkyl is substituted with NHC₁₋₆alkyl, for example NHmethyl, NHethyl, or NHpropyl, or for example NHCH₃.

Aspect 35: The compound of any one of Aspects 1 to 23, wherein X¹ is CR⁴R^{4′}.

Aspect 36: The compound of any one of Aspects 1 to 21, 23, or 24, wherein X² is N.

Aspect 37: The compound of any one of Aspects 1 to 21, 23, or 24, wherein X² is CR⁵.

Aspect 38: The compound of any one of the preceding Aspects, wherein R⁵ is:
H;
halo, such as F, Cl, Br, or I, for example F or Cl, or for example Cl; or
C₁₋₆alkyl, such as methyl, ethyl, propyl, butyl, pentyl, or hexyl, or for example methyl, or for example ethyl, or for example propyl such as n-propyl or i-propyl;

Aspect 39: The compound of any one of Aspects 1 to 23, wherein X² is CR⁵R^{5′}.

Aspect 40: The compound of Aspect 39, wherein R⁵ and R^{5′} are combined to form oxo.

Aspect 41: The compound of any one of Aspects 1 to 22, 24, or 25, wherein X³ is N.

Aspect 42: The compound of any one of Aspects 1 to 22, 24, or 25, wherein X³ is NR³ or CR³, for example NR³, or for example CR³.

Aspect 43: The compound of any one of Aspects 1 to 40 or 42, wherein R³ is:
H;
C₁₋₆alkyl, such as methyl, ethyl, propyl, butyl, pentyl, or hexyl, or for example methyl, or for example ethyl, or for example propyl such as n-propyl or i-propyl, or for example butyl such as n-butyl, i-butyl, or t-butyl, or for example i-butyl;
C₁₋₆haloalkyl, such as C₁₋₆fluoroalkyl, for example CF₃, CHF₂, CH₂F, CH₂CF₃, CH₂CF₂H, CH₂CH₂F, or CH₂CH₂CF₃, or for example CF₃, or for example CH₂CF₃, or for example CH₂CH₂CF₃; or
—C₀₋₆alkyl-C₃₋₈carbocyclyl, such as —C₁₋₆alkyl-C₃₋₈cycloalkyl or —CH₂—C₃₋₈cycloalkyl or —C₁₋₆alkyl-cyclopropyl, for example, CH₂-cyclopropyl, CH₂-cyclobutyl, CH₂-cyclopentyl, CH₂-cyclohexyl, or for example CH₂-cyclopropyl, or for example CH₂-cyclobutyl.

Aspect 44: The compound of Aspect 43, wherein R³ is —C₀₋₆alkyl-C₃₋₈carbocyclyl and the —C₀₋₆alkyl-C₃₋₈carbocyclyl is substituted with one or more halo, such as F, Cl, Br, or I, for example F, or for example Cl, or for example Br; or for example I.

Aspect 45: The compound of any one of Aspects 1 to 40 or 42 to 44, wherein R³ is:

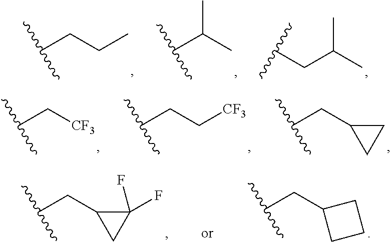

Aspect 46: The compound of Aspect 1, having the structure of Formula II:

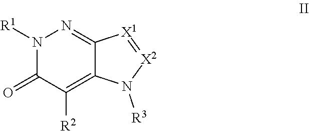

or a pharmaceutically acceptable salt thereof.

Aspect 47: The compound of Aspect 1, having the structure of Formula III:

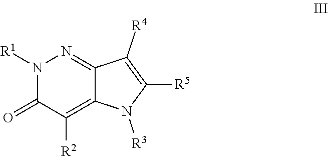

or a pharmaceutically acceptable salt thereof.

Aspect 48: The compound of Aspect 1, having the structure of Formula IV:

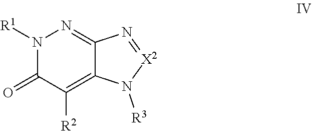

or a pharmaceutically acceptable salt thereof.

Aspect 49: The compound of Aspect 1, having the structure of Formula V:

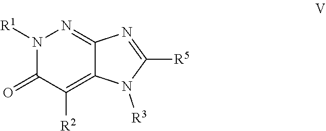

or a pharmaceutically acceptable salt thereof.

Aspect 50: The compound of Aspect 1, having the structure of Formula VI:

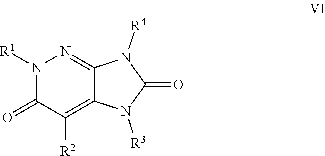

or a pharmaceutically acceptable salt thereof.

Aspect 51: The compound of Aspect 1, having the structure of Formula VII:

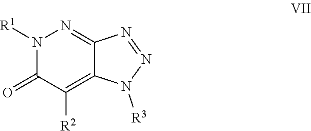

or a pharmaceutically acceptable salt thereof.

Aspect 52: The compound of Aspect 1, having the structure of Formula VIII:

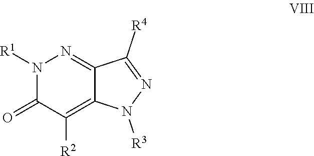

or a pharmaceutically acceptable salt thereof.

Aspect 53: The compound of Aspect 1, having the structure of Formula IX:

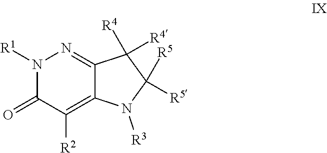

or a pharmaceutically acceptable salt thereof.

Aspect 54: The compound of Aspect 1, having the structure of Formula X:

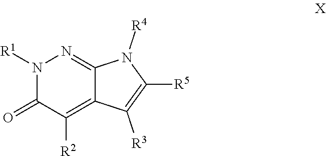

or a pharmaceutically acceptable salt thereof.

Aspect 55: The compound of Aspect 1, having the structure of Formula XIa, XIb, XIc:

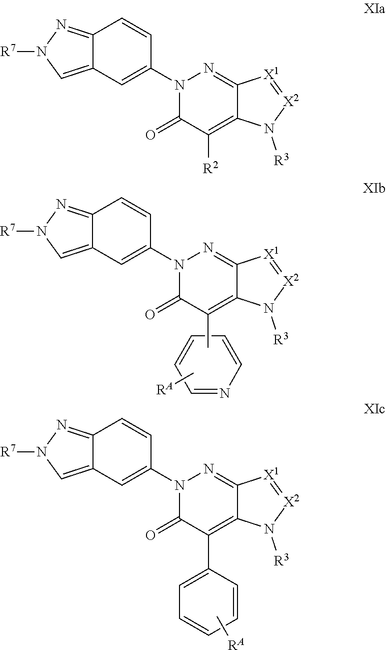

wherein R⁷ is H, C₁₋₆alkyl, deuterated C₁₋₆alkyl, —C₁₋₆alkyl-C₃₋₈carbocyclyl, C₁₋₆alkoxy, or deuterated C₁₋₆alkoxy;
or a pharmaceutically acceptable salt thereof.

Aspect 56: The compound of Aspect 1, having the structure of Formula XIIa, XIIb, or XIIc:

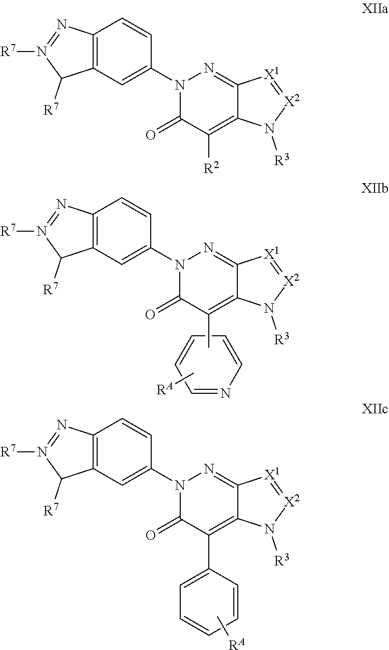

wherein each R⁷ is, independently, H, C₁₋₆alkyl, deuterated C₁₋₆alkyl, —C₁₋₆alkyl-C₃₋₈carbocyclyl, C₁₋₆alkoxy, or deuterated C₁₋₆alkoxy;
or a pharmaceutically acceptable salt thereof.

Aspect 57: The compound of Aspect 1, having the structure of Formula XIIIa, XIIIb. or XIIIc:

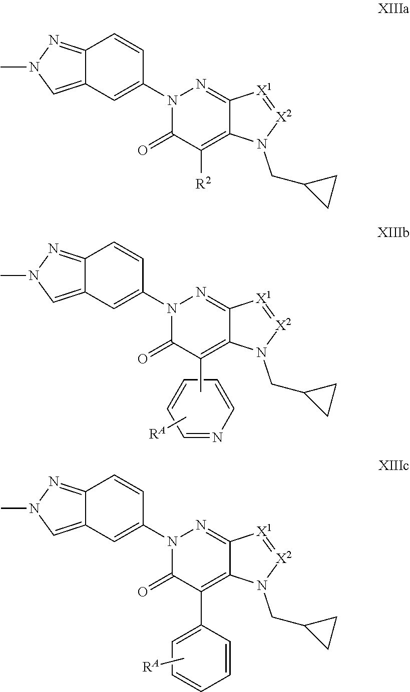

or a pharmaceutically acceptable salt thereof.

Aspect 58: The compound of Aspect 1, or a pharmaceutically acceptable salt thereof, that is:
Example 101: 5-(cyclopropylmethyl)-4-(4-(difluoromethoxy)phenyl)-2-(2-methyl-2H-indazol-5-yl)-2,5-dihydro-3H-pyrrolo[3,2-c]pyridazin-3-one;
Example 102: 5-(cyclopropylmethyl)-4-(4-(difluoromethoxy)phenyl)-2-(2-methyl-2H-indazol-5-yl)-3-oxo-3,5-dihydro-2H-pyrrolo[3,2-c]pyridazine-7-carbonitrile;
Example 103: 5-(cyclopropylmethyl)-2-(2-methyl-2H-indazol-5-yl)-4-(6-methylpyridin-3-yl)-2,5-dihydro-3H-pyrrolo[3,2-c]pyridazin-3-one;
Example 104: 5-(cyclopropylmethyl)-2-(2-methyl-2H-indazol-5-yl)-4-(6-methylpyridin-3-yl)-3-oxo-3,5-dihydro-2H-pyrrolo[3,2-c]pyridazine-7-carbonitrile;
Example 105: 4-(4-(difluoromethoxy)phenyl)-2-(2-methyl-2H-indazol-5-yl)-5-(3,3,3-trifluoropropyl)-2,5-dihydro-3H-pyrrolo[3,2-c]pyridazin-3-one;
Example 106: 5-(cyclopropylmethyl)-4-(6-cyclopropylpyridin-3-yl)-2-(2-methyl-2H-indazol-5-yl)-2,5-dihydro-3H-pyrrolo[3,2-c]pyridazin-3-one;
Example 107: 5-(cyclopropylmethyl)-4-(6-cyclopropylpyridin-3-yl)-2-(2-methyl-2H-indazol-5-yl)-3-oxo-3,5-dihydro-2H-pyrrolo[3,2-c]pyridazine-7-carbonitrile;
Example 108: 2-(2-methyl-2H-indazol-5-yl)-4-(6-methylpyridin-3-yl)-3-oxo-5-(3,3,3-trifluoropropyl)-3,5-dihydro-2H-pyrrolo[3,2-c]pyridazine-7-carbonitrile;
Example 109: 4-(6-cyclopropylpyridin-3-yl)-2-(2-methyl-2H-indazol-5-yl)-3-oxo-5-(3,3,3-trifluoropropyl)-3,5-dihydro-2H-pyrrolo[3,2-c]pyridazine-7-carbonitrile;
Example 110: 5-(cyclopropylmethyl)-4-(6-methoxypyridin-3-yl)-2-(2-methyl-2H-indazol-5-yl)-2,5-dihydro-3H-pyrrolo[3,2-c]pyridazin-3-one;
Example 111: 5-(cyclopropylmethyl)-4-(6-methoxypyridin-3-yl)-2-(2-methyl-2H-indazol-5-yl)-3-oxo-3,5-dihydro-2H-pyrrolo[3,2-c]pyridazine-7-carbonitrile;
Example 112: 4-(4-chlorophenyl)-5-(cyclopropylmethyl)-2-(2-methyl-2H-indazol-5-yl)-2,5-dihydro-3H-pyrrolo[3,2-c]pyridazin-3-one;
Example 113: 5-(cyclopropylmethyl)-4-(6-(difluoromethoxy)pyridin-3-yl)-2-(2-methyl-2H-indazol-5-yl)-2,5-dihydro-3H-pyrrolo[3,2-c]pyridazin-3-one;
Example 114: 5-(cyclopropylmethyl)-4-(6-(difluoromethoxy)pyridin-3-yl)-2-(2-methyl-2H-indazol-5-yl)-3-oxo-3,5-dihydro-2H-pyrrolo[3,2-c]pyridazine-7-carbonitrile;
Example 115: 4-(4-(difluoromethoxy)phenyl)-5-isobutyl-2-(2-methyl-2H-indazol-5-yl)-2,5-dihydro-3H-pyrrolo[3,2-c]pyridazin-3-one;
Example 116: 4-(4-(difluoromethoxy)phenyl)-5-isobutyl-2-(2-methyl-2H-indazol-5-yl)-3-oxo-3,5-dihydro-2H-pyrrolo[3,2-c]pyridazine-7-carbonitrile;
Example 117: 5-(cyclopropylmethyl)-4-(6-cyclopropylpyridin-3-yl)-7-iodo-2-(2-methyl-2H-indazol-5-yl)-2,5-dihydro-3H-pyrrolo[3,2-c]pyridazin-3-one;
Example 118: 5-(cyclopropylmethyl)-2-(2-methyl-2H-indazol-5-yl)-4-(6-(trifluoromethyl)pyridin-3-yl)-2,5-dihydro-3H-pyrrolo[3,2-c]pyridazin-3-one;
Example 119: 5-(cyclopropylmethyl)-2-(2-methyl-2H-indazol-5-yl)-3-oxo-4-(6-(trifluoromethyl)pyridin-3-yl)-3,5-dihydro-2H-pyrrolo[3,2-c]pyridazine-7-carbonitrile;
Example 120: 4-(6-(1-cyanocyclopropyl)pyridin-3-yl)-5-(cyclopropylmethyl)-2-(2-methyl-2H-indazol-5-yl)-3-oxo-3,5-dihydro-2H-pyrrolo[3,2-c]pyridazine-7-carbonitrile;
Example 121: 4-(6-cyclopropylpyridin-3-yl)-5-isobutyl-2-(2-methyl-2H-indazol-5-yl)-3-oxo-3,5-dihydro-2H-pyrrolo[3,2-c]pyridazine-7-carbonitrile;
Example 122: 5-(cyclopropylmethyl)-4-(6-cyclopropylpyridin-3-yl)-2-(4-(methoxy-d3)phenyl)-2,5-dihydro-3H-pyrrolo[3,2-c]pyridazin-3-one;
Example 123: 5-(cyclopropylmethyl)-4-(6-(difluoromethyl)pyridin-3-yl)-2-(2-methyl-2H-indazol-5-yl)-2,5-dihydro-3H-pyrrolo[3,2-c]pyridazin-3-one;
Example 124: 5-(cyclopropylmethyl)-4-(6-cyclopropylpyridin-3-yl)-2-(4-(methoxy-d3)phenyl)-3-oxo-3,5-dihydro-2H-pyrrolo[3,2-c]pyridazine-7-carbonitrile;
Example 125: 5-(cyclopropylmethyl)-2-(2-methyl-2H-indazol-5-yl)-4-(5-(trifluoromethyl)pyridin-3-yl)-2,5-dihydro-3H-pyrrolo[3,2-c]pyridazin-3-one;
Example 126: 4-(5-chloropyridin-3-yl)-5-(cyclopropylmethyl)-2-(2-methyl-2H-indazol-5-yl)-2,5-dihydro-3H-pyrrolo[3,2-c]pyridazin-3-one;
Example 127: 4-(6-cyclopropylpyridin-3-yl)-2-(2-methyl-2H-indazol-5-yl)-3-oxo-5-propyl-3,5-dihydro-2H-pyrrolo[3,2-c]pyridazine-7-carbonitrile;
Example 128: 5-(cyclopropylmethyl)-4-(6-isopropylpyridin-3-yl)-2-(2-methyl-2H-indazol-5-yl)-3-oxo-3,5-dihydro-2H-pyrrolo[3,2-c]pyridazine-7-carbonitrile;
Example 129: 5-(cyclopropylmethyl)-4-(6-isopropylpyridin-3-yl)-2-(2-methyl-2H-indazol-5-yl)-2,5-dihydro-3H-pyrrolo[3,2-c]pyridazin-3-one;
Example 130: 4-(3-cyano-1H-indol-5-yl)-5-(cyclopropylmethyl)-2-(2-methyl-2H-indazol-5-yl)-3-oxo-3,5-dihydro-2H-pyrrolo[3,2-c]pyridazine-7-carbonitrile;
Example 131: 5-(cyclopropylmethyl)-2-(4-(methoxy-d3)phenyl)-4-(6-methylpyridin-3-yl)-3-oxo-3,5-dihydro-2H-pyrrolo[3,2-c]pyridazine-7-carbonitrile;
Example 132: 5-(cyclopropylmethyl)-4-(6-(difluoromethyl)pyridin-3-yl)-2-(4-(methoxy-d3)phenyl)-2,5-dihydro-3H-pyrrolo[3,2-c]pyridazin-3-one;
Example 133: 4-(6-cyclopropylpyridin-3-yl)-2-(2-methyl-2H-indazol-5-yl)-3-oxo-5-(2,2,2-trifluoroethyl)-3,5-dihydro-2H-pyrrolo[3,2-c]pyridazine-7-carbonitrile;
Example 134: 5-(cyclopropylmethyl)-2-(4-(methoxy-d3)phenyl)-4-(6-methylpyridin-3-yl)-2,5-dihydro-3H-pyrrolo[3,2-c]pyridazin-3-one;
Example 135: 5-(cyclopropylmethyl)-4-(6-ethylpyridin-3-yl)-2-(2-methyl-2H-indazol-5-yl)-2,5-dihydro-3H-pyrrolo[3,2-c]pyridazin-3-one;
Example 136: 5-(cyclopropylmethyl)-4-(6-ethylpyridin-3-yl)-2-(2-methyl-2H-indazol-5-yl)-3-oxo-3,5-dihydro-2H-pyrrolo[3,2-c]pyridazine-7-carbonitrile;
Example 137: 5-(cyclopropylmethyl)-4-(6-(difluoromethyl)pyridin-3-yl)-2-(4-(methoxy-d3)phenyl)-3-oxo-3,5-dihydro-2H-pyrrolo[3,2-c]pyridazine-7-carbonitrile;
Example 138: 5-(cyclopropylmethyl)-4-(4-cyclopropylphenyl)-2-(2-methyl-2H-indazol-5-yl)-3-oxo-3,5-dihydro-2H-pyrrolo[3,2-c]pyridazine-7-carbonitrile;
Example 139: 4-(4-chlorophenyl)-5-(cyclopropylmethyl)-2-(2-methyl-2H-indazol-5-yl)-3-oxo-3,5-dihydro-2H-pyrrolo[3,2-c]pyridazine-7-carbonitrile;
Example 140: 5-(cyclopropylmethyl)-4-(6-(difluoromethyl)pyridin-3-yl)-2-(1,2-dimethyl-1H-benzo[d]imidazol-6-yl)-2,5-dihydro-3H-pyrrolo[3,2-c]pyridazin-3-one;
Example 141: 5-(cyclopropylmethyl)-2-(1,2-dimethyl-1H-benzo[d]imidazol-6-yl)-4-(6-methylpyridin-3-yl)-2,5-dihydro-3H-pyrrolo[3,2-c]pyridazin-3-one;
Example 142: 5-(cyclopropylmethyl)-2-(1,2-dimethyl-1H-benzo[d]imidazol-6-yl)-4-(6-ethylpyridin-3-yl)-2,5-dihydro-3H-pyrrolo[3,2-c]pyridazin-3-one;
Example 143: 5-(cyclopropylmethyl)-4-(6-(difluoromethyl)pyridin-3-yl)-2-(2-methyl-2H-indazol-5-yl)-3-oxo-3,5-dihydro-2H-pyrrolo[3,2-c]pyridazine-7-carbonitrile;
Example 144: 5-(5-(cyclopropylmethyl)-2-(2-methyl-2H-indazol-5-yl)-3-oxo-3,5-dihydro-2H-pyrrolo[3,2-c]pyridazin-4-yl)-N-methylpicolinamide;
Example 145: 2-(2-methyl-2H-indazol-5-yl)-4-(6-methylpyridin-3-yl)-3-oxo-5-propyl-3,5-dihydro-2H-pyrrolo[3,2-c]pyridazine-7-carbonitrile;
Example 146: 4-(6-cyclopropylpyridin-3-yl)-5-isopropyl-2-(2-methyl-2H-indazol-5-yl)-3-oxo-3,5-dihydro-2H-pyrrolo[3,2-c]pyridazine-7-carbonitrile;
Example 147: 5-(cyclopropylmethyl)-4-(6-(difluoromethyl)pyridin-3-yl)-2-(1,2-dimethyl-1H-benzo[d]imidazol-6-yl)-3-oxo-3,5-dihydro-2H-pyrrolo[3,2-c]pyridazine-7-carbonitrile;
Example 148: 5-(cyclopropylmethyl)-4-(6-cyclopropylpyridin-3-yl)-2-(1,2-dimethyl-1H-benzo[d]imidazol-6-yl)-3-oxo-3,5-dihydro-2H-pyrrolo[3,2-c]pyridazine-7-carbonitrile;
Example 149: 4-(4-cyanophenyl)-5-(cyclopropylmethyl)-2-(2-methyl-2H-indazol-5-yl)-3-oxo-3,5-dihydro-2H-pyrrolo[3,2-c]pyridazine-7-carbonitrile;
Example 150: 5-(cyclopropylmethyl)-4-(4-cyclopropylphenyl)-2-(2-methyl-2H-indazol-5-yl)-2,5-dihydro-3H-pyrrolo[3,2-c]pyridazin-3-one;
Example 151: 5-(cyclopropylmethyl)-2-(1,2-dimethyl-1H-benzo[d]imidazol-6-yl)-4-(6-methylpyridin-3-yl)-3-oxo-3,5-dihydro-2H-pyrrolo[3,2-c]pyridazine-7-carbonitrile;
Example 152: 5-(cyclopropylmethyl)-2-(1,2-dimethyl-1H-benzo[d]imidazol-6-yl)-4-(6-ethylpyridin-3-yl)-3-oxo-3,5-dihydro-2H-pyrrolo[3,2-c]pyridazine-7-carbonitrile;
Example 153: 5-(5-(cyclopropylmethyl)-2-(2-methyl-2H-indazol-5-yl)-3-oxo-3,5-dihydro-2H-pyrrolo[3,2-c]pyridazin-4-yl)picolinonitrile;
Example 154: 4-(5-(cyclopropylmethyl)-2-(2-methyl-2H-indazol-5-yl)-3-oxo-3,5-dihydro-2H-pyrrolo[3,2-c]pyridazin-4-yl)benzonitrile;
Example 155: 5-(5-(cyclopropylmethyl)-2-(2-methyl-2H-indazol-5-yl)-3-oxo-3,5-dihydro-2H-pyrrolo[3,2-c]pyridazin-4-yl)picolinamide;
Example 156: 5-(cyclopropylmethyl)-4-(6-cyclopropylpyridin-3-yl)-2-(1,2-dimethyl-1H-benzo[d]imidazol-6-yl)-2,5-dihydro-3H-pyrrolo[3,2-c]pyridazin-3-one;
Example 157: 5-(cyclopropylmethyl)-4-(6-(hydroxymethyl)pyridin-3-yl)-2-(2-methyl-2H-indazol-5-yl)-2,5-dihydro-3H-pyrrolo[3,2-c]pyridazin-3-one;
Example 158: 5-(cyclopropylmethyl)-4-(6-(hydroxymethyl)pyridin-3-yl)-2-(2-methyl-2H-indazol-5-yl)-3-oxo-3,5-dihydro-2H-pyrrolo[3,2-c]pyridazine-7-carbonitrile;
Example 159: 5-(cyclobutylmethyl)-4-(6-cyclopropylpyridin-3-yl)-2-(2-methyl-2H-indazol-5-yl)-3-oxo-3,5-dihydro-2H-pyrrolo[3,2-c]pyridazine-7-carbonitrile;
Example 160: 5-(cyclopropylmethyl)-2-(2-(cyclopropylmethyl)-2H-indazol-5-yl)-4-(6-cyclopropylpyridin-3-yl)-3-oxo-3,5-dihydro-2H-pyrrolo[3,2-c]pyridazine-7-carbonitrile;
Example 161: 5-(cyclopropylmethyl)-4-(4-(methoxy-d3)phenyl)-2-(2-methyl-2H-indazol-5-yl)-3-oxo-3,5-dihydro-2H-pyrrolo[3,2-c]pyridazine-7-carbonitrile;
Example 162: 5-(cyclopropylmethyl)-4-(6-cyclopropylpyridin-3-yl)-N-methyl-2-(2-methyl-2H-indazol-5-yl)-3-oxo-2H,3H,5H-pyrrolopyridazine-7-carboxamide;
Example 163: 5-(cyclopropylmethyl)-4-(6-cyclopropylpyridin-3-yl)-2-(2-methyl-2H-indazol-5-yl)-3-oxo-3,5-dihydro-2H-pyrrolo[3,2-c]pyridazine-7-carboxylic acid;
Example 164: 5-(cyclopropylmethyl)-4-(6-cyclopropylpyridin-3-yl)-N,N-dimethyl-2-(2-methyl-2H-indazol-5-yl)-3-oxo-3,5-dihydro-2H-pyrrolo[3,2-c]pyridazine-7-carboxamide;
Example 165: 5-(cyclopropylmethyl)-4-(6-cyclopropylpyridin-3-yl)-2-(2-methyl-2H-indazol-5-yl)-3-oxo-3,5-dihydro-2H-pyrrolo[3,2-c]pyridazine-7-carboxamide;
Example 166: 5-(cyclopropylmethyl)-4-(4-cyclopropylphenyl)-2-(2-methyl-2H-indazol-5-yl)-3-oxo-3,5-dihydro-2H-pyrrolo[3,2-c]pyridazine-7-carboxamide;
Example 167: 5-(cyclopropylmethyl)-4-(4-cyclopropylphenyl)-N-methyl-2-(2-methyl-2H-indazol-5-yl)-3-oxo-3,5-dihydro-2H-pyrrolo[3,2-c]pyridazine-7-carboxamide;
Example 168: 5-(cyclopropylmethyl)-4-(4-(difluoromethoxy)phenyl)-2-(2-methyl-2H-indazol-5-yl)-3-oxo-3,5-dihydro-2H-pyrrolo[3,2-c]pyridazine-7-carboxamide;
Example 169: 5-(cyclopropylmethyl)-4-(4-(difluoromethoxy)phenyl)-N-methyl-2-(2-methyl-2H-indazol-5-yl)-3-oxo-3,5-dihydro-2H-pyrrolo[3,2-c]pyridazine-7-carboxamide;
Example 170: 4-(4-chlorophenyl)-5-(cyclopropylmethyl)-2-(2-methyl-2H-indazol-5-yl)-3-oxo-3,5-dihydro-2H-pyrrolo[3,2-c]pyridazine-7-carboxamide;
Example 171: 4-(4-chlorophenyl)-5-(cyclopropylmethyl)-N-methyl-2-(2-methyl-2H-indazol-5-yl)-3-oxo-3,5-dihydro-2H-pyrrolo[3,2-c]pyridazine-7-carboxamide;
Example 172: 5-(cyclopropylmethyl)-4-(6-cyclopropylpyridin-3-yl)-N-methyl-2-(2-(methyl-d3)-2H-indazol-5-yl)-3-oxo-3,5-dihydro-2H-pyrrolo[3,2-c]pyridazine-7-carboxamide;
Example 173: 5-(cyclopropylmethyl)-4-(4-(methoxy-d3)phenyl)-2-(2-methyl-2H-indazol-5-yl)-3-oxo-3,5-dihydro-2H-pyrrolo[3,2-c]pyridazine-7-carboxamide;
Example 174: 5-(cyclopropylmethyl)-4-(4-(methoxy-d3)phenyl)-N-methyl-2-(2-methyl-2H-indazol-5-yl)-3-oxo-3,5-dihydro-2H-pyrrolo[3,2-c]pyridazine-7-carboxamide;
Example 175: 5-(cyclopropylmethyl)-2-(2-methyl-2H-indazol-5-yl)-4-(6-methylpyridin-3-yl)-3-oxo-3,5-dihydro-2H-pyrrolo[3,2-c]pyridazine-7-carboxamide;
Example 176: 5-(cyclopropylmethyl)-4-(4-methoxyphenyl)-N-methyl-2-(2-methyl-2H-indazol-5-yl)-3-oxo-3,5-dihydro-2H-pyrrolo[3,2-c]pyridazine-7-carboxamide;
Example 177: 5-(cyclopropylmethyl)-N-methyl-2-(2-methyl-2H-indazol-5-yl)-4-(6-methylpyridin-3-yl)-3-oxo-3,5-dihydro-2H-pyrrolo[3,2-c]pyridazine-7-carboxamide;
Example 178: 5-(cyclopropylmethyl)-4-(6-cyclopropylpyridin-3-yl)-N,N-dimethyl-2-(2-methyl-2H-indazol-5-yl)-3-oxo-3,5-dihydro-2H-pyrrolo[3,2-c]pyridazine-7-sulfonamide;
Example 179: 5-(cyclopropylmethyl)-4-(4-cyclopropylphenyl)-N-methyl-2-(2-methyl-2H-indazol-5-yl)-3-oxo-3,5-dihydro-2H-pyrrolo[3,2-c]pyridazine-7-sulfonamide;
Example 180: 5-(cyclopropylmethyl)-4-(4-cyclopropylphenyl)-2-(2-methyl-2H-indazol-5-yl)-3-oxo-3,5-dihydro-2H-pyrrolo[3,2-c]pyridazine-7-sulfonamide;
Example 181: 5-(cyclopropylmethyl)-4-(6-cyclopropylpyridin-3-yl)-N-methyl-2-(2-methyl-2H-indazol-5-yl)-3-oxo-3,5-dihydro-2H-pyrrolo[3,2-c]pyridazine-7-sulfonamide;
Example 182: 5-(cyclopropylmethyl)-N-methyl-2-(2-methyl-2H-indazol-5-yl)-4-(6-methylpyridin-3-yl)-3-oxo-3,5-dihydro-2H-pyrrolo[3,2-c]pyridazine-7-sulfonamide;
Example 183: 5-(cyclopropylmethyl)-4-(4-(difluoromethoxy)phenyl)-N-methyl-2-(2-methyl-2H-indazol-5-yl)-3-oxo-3,5-dihydro-2H-pyrrolo[3,2-c]pyridazine-7-sulfonamide;
Example 184: 5-(cyclopropylmethyl)-4-(4-(difluoromethoxy)phenyl)-2-(2-methyl-2H-indazol-5-yl)-3-oxo-3,5-dihydro-2H-pyrrolo[3,2-c]pyridazine-7-sulfonamide;
Example 185: 5-(cyclopropylmethyl)-4-(6-cyclopropylpyridin-3-yl)-2-(2-methyl-2H-indazol-5-yl)-3-oxo-3,5-dihydro-2H-pyrrolo[3,2-c]pyridazine-7-sulfonamide;
Example 223: 5-(cyclopropylmethyl)-4-(4-(difluoromethoxy)phenyl)-2-(1-methyl-6-oxo-1,6-dihydropyridin-3-yl)-2,5-dihydro-3H-pyrrolo[3,2-c]pyridazin-3-one;
Example 224: 2-(benzo[d]thiazol-6-yl)-5-(cyclopropylmethyl)-4-(4-(difluoromethoxy)phenyl)-2,5-dihydro-3H-pyrrolo[3,2-c]pyridazin-3-one;
Example 225: 5-(cyclopropylmethyl)-4-(4-(difluoromethoxy)phenyl)-2-(quinolin-6-yl)-2,5-dihydro-3H-pyrrolo[3,2-c]pyridazin-3-one;
Example 226: 5-(cyclopropylmethyl)-4-(4-(difluoromethoxy)phenyl)-2-(pyridin-4-yl)-2,5-dihydro-3H-pyrrolo[3,2-c]pyridazin-3-one;
Example 227: 5-(cyclopropylmethyl)-4-(4-(difluoromethoxy)phenyl)-2-(4-methyl-3,4-dihydro-2H-benzo[b][1,4]oxazin-6-yl)-2,5-dihydro-3H-pyrrolo[3,2-c]pyridazin-3-one;
Example 228: 2-(4-aminophenyl)-5-(cyclopropylmethyl)-4-(4-(difluoromethoxy)phenyl)-2,5-dihydro-3H-pyrrolo[3,2-c]pyridazin-3-one;
Example 229: 5-(cyclopropylmethyl)-4-(4-(difluoromethoxy)phenyl)-2-(4-methoxyphenyl)-2,5-dihydro-3H-pyrrolo[3,2-c]pyridazin-3-one;
Example 230: 5-(cyclopropylmethyl)-4-(4-(difluoromethoxy)phenyl)-2-(4-methoxycyclohex-1-en-1-yl)-2,5-dihydro-3H-pyrrolo[3,2-c]pyridazin-3-one;
Example 231: 5-(cyclopropylmethyl)-4-(4-(difluoromethoxy)phenyl)-2-(1H-pyrazol-4-yl)-2,5-dihydro-3H-pyrrolo[3,2-c]pyridazin-3-one;
Example 232: 5-(cyclopropylmethyl)-4-(6-cyclopropylpyridin-3-yl)-2-(1-methyl-1,2,3,6-tetrahydropyridin-4-yl)-2,5-dihydro-3H-pyrrolo[3,2-c]pyridazin-3-one;
Example 233: 7-chloro-5-(cyclopropylmethyl)-4-(6-cyclopropylpyridin-3-yl)-2-(2-methyl-2H-indazol-5-yl)-2,5-dihydro-3H-pyrrolo[3,2-c]pyridazin-3-one;
Example 234: 5-(cyclopropylmethyl)-4-(6-cyclopropylpyridin-3-yl)-7-methyl-2-(2-methyl-2H-indazol-5-yl)-2,5-dihydro-3H-pyrrolo[3,2-c]pyridazin-3-one;
Example 235: 5-(cyclopropylmethyl)-4-(6-cyclopropylpyridin-3-yl)-7-fluoro-2-(2-methyl-2H-indazol-5-yl)-2,5-dihydro-3H-pyrrolo[3,2-c]pyridazin-3-one;
Example 236: 7-acetyl-5-(cyclopropylmethyl)-4-(6-cyclopropylpyridin-3-yl)-2-(2-methyl-2H-indazol-5-yl)-2,5-dihydro-3H-pyrrolo[3,2-c]pyridazin-3-one;
Example 237: 5-(cyclopropylmethyl)-7-fluoro-2-(2-methyl-2H-indazol-5-yl)-4-(6-methylpyridin-3-yl)-2,5-dihydro-3H-pyrrolo[3,2-c]pyridazin-3-one;
Example 238: 7-bromo-5-(cyclopropylmethyl)-4-(6-cyclopropylpyridin-3-yl)-2-(2-methyl-2H-indazol-5-yl)-2,5-dihydro-3H-pyrrolo[3,2-c]pyridazin-3-one;
Example 239: 5-(cyclopropylmethyl)-4-(6-cyclopropylpyridin-3-yl)-7-(hydroxymethyl)-2-(2-methyl-2H-indazol-5-yl)-2,5-dihydro-3H-pyrrolo[3,2-c]pyridazin-3-one;
Example 240: 5-(cyclopropylmethyl)-4-(6-cyclopropylpyridin-3-yl)-7-(difluoromethyl)-2-(2-methyl-2H-indazol-5-yl)-2,5-dihydro-3H-pyrrolo[3,2-c]pyridazin-3-one;
Example 241: 5-(cyclopropylmethyl)-4-(6-cyclopropylpyridin-3-yl)-2-(2-methyl-3-(trifluoromethyl)-2H-indazol-5-yl)-2,5-dihydro-3H-pyrrolo[3,2-c]pyridazin-3-one:
Example 242: 5-(cyclopropylmethyl)-4-(6-cyclopropylpyridin-3-yl)-2-(2-methyl-3-(trifluoromethyl)-2H-indazol-5-yl)-7-(trifluoromethyl)-2,5-dihydro-3H-pyrrolo[3,2-c]pyridazin-3-one;
Example 243: 5-(cyclopropylmethyl)-4-(6-cyclopropylpyridin-3-yl)-2-(2-methyl-2H-indazol-5-yl)-7-((trimethylsilyl)ethynyl)-2,5-dihydro-3H-pyrrolo[3,2-c]pyridazin-3-one;
Example 244: 5-(cyclopropylmethyl)-4-(6-cyclopropylpyridin-3-yl)-7-ethynyl-2-(2-methyl-2H-indazol-5-yl)-2,5-dihydro-3H-pyrrolo[3,2-c]pyridazin-3-one;
Example 245: 5-(cyclopropylmethyl)-4-(6-cyclopropylpyridin-3-yl)-2-(2-methyl-2H-indazol-5-yl)-7-(3,3,3-trifluoroprop-1-yn-1-yl)-2,5-dihydro-3H-pyrrolo[3,2-c]pyridazin-3-one;
Example 246: 5-(cyclopropylmethyl)-4-(6-cyclopropylpyridin-3-yl)-2-(2-methyl-2H-indazol-5-yl)-7-(trifluoromethyl)-2,5-dihydro-3H-pyrrolo[3,2-c]pyridazin-3-one;
Example 247: 5-(cyclopropylmethyl)-4-(6-cyclopropylpyridin-3-yl)-7-ethyl-2-(2-methyl-2H-indazol-5-yl)-2,5-dihydro-3H-pyrrolo[3,2-c]pyridazin-3-one;
Example 248: 5-(cyclopropylmethyl)-4-(6-cyclopropylpyridin-3-yl)-7-isopropyl-2-(2-methyl-2H-indazol-5-yl)-2,5-dihydro-3H-pyrrolo[3,2-c]pyridazin-3-one;
Example 249: (R)-5-(cyclopropylmethyl)-4-(6-cyclopropylpyridin-3-yl)-7-(1-hydroxyethyl)-2-(2-methyl-2H-indazol-5-yl)-2,5-dihydro-3H-pyrrolo[3,2-c]pyridazin-3-one;
Example 250: (S)-5-(cyclopropylmethyl)-4-(6-cyclopropylpyridin-3-yl)-7-(1-hydroxyethyl)-2-(2-methyl-2H-indazol-5-yl)-2,5-dihydro-3H-pyrrolo[3,2-c]pyridazin-3-one;
Example 251: 6-chloro-5-(cyclopropylmethyl)-4-(6-cyclopropylpyridin-3-yl)-2-(2-methyl-2H-indazol-5-yl)-3-oxo-3,5-dihydro-2H-pyrrolo[3,2-c]pyridazine-7-carbonitrile;
Example 252: (S)-5-(cyclopropylmethyl)-4-(6-(2,2-difluorocyclopropyl)pyridin-3-yl)-2-(2-methyl-2H-indazol-5-yl)-3-oxo-3,5-dihydro-2H-pyrrolo[3,2-c]pyridazine-7-carbonitrile;
Example 253: (R)-5-(cyclopropylmethyl)-4-(6-(2,2-difluorocyclopropyl)pyridin-3-yl)-2-(2-methyl-2H-indazol-5-yl)-3-oxo-3,5-dihydro-2H-pyrrolo[3,2-c]pyridazine-7-carbonitrile;
Example 254: 5-(cyclopropylmethyl)-2-(2-methyl-2H-indazol-5-yl)-4-[6-(1-methylcyclopropyl)pyridin-3-yl]-3-oxo-2H,3H,5H-pyrrolo[3,2-c]pyridazine-7-carbonitrile;
Example 255: 5-(cyclopropylmethyl)-7-(hydroxymethyl)-2-(2-methyl-2H-indazol-5-yl)-4-(6-methylpyridin-3-yl)-2,5-dihydro-3H-pyrrolo[3,2-c]pyridazin-3-one;
Example 256: 5-(cyclopropylmethyl)-4-(6-(1-hydroxycyclopropyl)pyridin-3-yl)-2-(2-methyl-2H-indazol-5-yl)-3-oxo-3,5-dihydro-2H-pyrrolo[3,2-c]pyridazine-7-carbonitrile;
Example 257: 5-(cyclopropylmethyl)-4-(6-(3-fluoroprop-1-en-2-yl)pyridin-3-yl)-2-(2-methyl-2H-indazol-5-yl)-3-oxo-3,5-dihydro-2H-pyrrolo[3,2-c]pyridazine-7-carbonitrile;
Example 258: 5-(cyclopropylmethyl)-2-(2-methyl-2H-indazol-5-yl)-4-(6-methylpyridin-3-yl)-7-(trifluoromethyl)-2,5-dihydro-3H-pyrrolo[3,2-c]pyridazin-3-one;
Example 259: 2-(5-(cyclopropylmethyl)-4-(6-cyclopropylpyridin-3-yl)-2-(2-methyl-2H-indazol-5-yl)-3-oxo-3,5-dihydro-2H-pyrrolo[3,2-c]pyridazin-7-yl)acetonitrile;
Example 261: 5-(cyclopropylmethyl)-4-(6-cyclopropylpyridin-3-yl)-2-(2-methyl-2H-indazol-5-yl)-7-(methylsulfonyl)-2,5-dihydro-3H-pyrrolo[3,2-c]pyridazin-3-one;
Example 262: 5-(cyclopropylmethyl)-4-(4-cyclopropylphenyl)-2-(2-methyl-2H-indazol-5-yl)-7-(methylsulfonyl)-2,5-dihydro-3H-pyrrolo[3,2-c]pyridazin-3-one;
Example 263: 5-(cyclopropylmethyl)-4-(6-cyclopropylpyridin-3-yl)-6-methyl-2-(2-methyl-2H-indazol-5-yl)-2,5-dihydro-3H-pyrrolo[3,2-c]pyridazin-3-one;
Example 264: 5-(cyclopropylmethyl)-4-(4-(difluoromethoxy)phenyl)-6-methyl-2-(2-methyl-2H-indazol-5-yl)-3-oxo-3,5-dihydro-2H-pyrrolo[3,2-c]pyridazine-7-carbonitrile;
Example 265: 5-(cyclopropylmethyl)-4-(6-cyclopropylpyridin-3-yl)-6-methyl-2-(2-methyl-2H-indazol-5-yl)-3-oxo-2H,3H,5H-pyrrolopyridazine-7-carbonitrile;
Example 266: 5-(cyclopropylmethyl)-6-methyl-2-(2-methyl-2H-indazol-5-yl)-4-(6-methylpyridin-3-yl)-3-oxo-2H,3H,5H-pyrrolopyridazine-7-carbonitrile;
Example 267: 5-(cyclopropylmethyl)-4-(6-cyclopropylpyridin-3-yl)-2-(2-methyl-2H-indazol-5-yl)-7-((methylamino)methyl)-2,5-dihydro-3H-pyrrolo[3,2-c]pyridazin-3-one;
Example 268: (S)-4-(6-cyclopropylpyridin-3-yl)-5-((2,2-difluorocyclopropyl)methyl)-2-(2-methyl-2H-indazol-5-yl)-3-oxo-3,5-dihydro-2H-pyrrolo[3,2-c]pyridazine-7-carbonitrile;
Example 269: (R)-4-(6-cyclopropylpyridin-3-yl)-5-((2,2-difluorocyclopropyl)methyl)-2-(2-methyl-2H-indazol-5-yl)-3-oxo-3,5-dihydro-2H-pyrrolo[3,2-c]pyridazine-7-carbonitrile; or
Example 270: 5-(cyclopropylmethyl)-4-(6-cyclopropylpyridin-3-yl)-2-(2-(methyl-d3)-2H-indazol-5-yl)-3-oxo-3,5-dihydro-2H-pyrrolo[3,2-c]pyridazine-7-carbonitrile.
Aspect 59: The compound of Aspect 1, or a pharmaceutically acceptable salt thereof, that is:
Example 186: 5-(cyclopropylmethyl)-4-[4-(difluoromethoxy)phenyl]-2-(2-methyl-2H-indazol-5-yl)-2H,3H,5H-imidazo[4,5-c]pyridazin-3-one;
Example 187: 5-(cyclopropylmethyl)-2-(2-methyl-2H-indazol-5-yl)-4-(6-methylpyridin-3-yl)-2,5-dihydro-3H-imidazo[4,5-c]pyridazin-3-one;
Example 188: 4-(4-chlorophenyl)-5-(cyclopropylmethyl)-2-(2-methyl-2H-indazol-5-yl)-2,5-dihydro-3H-imidazo[4,5-c]pyridazin-3-one;
Example 189: 5-(cyclopropylmethyl)-4-(6-(difluoromethoxy)pyridin-3-yl)-2-(2-methyl-2H-indazol-5-yl)-2,5-dihydro-3H-imidazo[4,5-c]pyridazin-3-one;
Example 190: 5-(cyclopropylmethyl)-4-(6-cyclopropylpyridin-3-yl)-2-(2-methyl-2H-indazol-5-yl)-2,5-dihydro-3H-imidazo[4,5-c]pyridazin-3-one;
Example 191: 5-(cyclopropylmethyl)-4-(4-(difluoromethoxy)phenyl)-6-methyl-2-(2-methyl-2H-indazol-5-yl)-2,5-dihydro-3H-imidazo[4,5-c]pyridazin-3-one;
Example 192: 5-(cyclopropylmethyl)-4-(4-cyclopropylphenyl)-2-(2-methyl-2H-indazol-5-yl)-2,5-dihydro-3H-imidazo[4,5-c]pyridazin-3-one;
Example 193: 5-(cyclopropylmethyl)-4-(4-methoxyphenyl)-2-(2-methyl-2H-indazol-5-yl)-2,5-dihydro-3H-imidazo[4,5-c]pyridazin-3-one;
Example 194: 5-(cyclopropylmethyl)-4-(4-(methoxy-d3)phenyl)-2-(2-methyl-2H-indazol-5-yl)-2,5-dihydro-3H-imidazo[4,5-c]pyridazin-3-one; or
Example 195: 5-(cyclopropylmethyl)-2-(1,2-dimethyl-1H-benzo[d]imidazol-6-yl)-4-(4-methoxyphenyl)-2,5-dihydro-3H-imidazo[4,5-c]pyridazin-3-one.

Aspect 60: The compound of Aspect 1, or a pharmaceutically acceptable salt thereof, that is:
Example 196: 5-(cyclopropylmethyl)-4-[4-(difluoromethoxy)phenyl]-7-methyl-2-(2-methyl-2H-indazol-5-yl)-2H,3H,5H,6H,7H-imidazolidino[4,5-c]pyridazine-3,6-dione;
Example 197: 5-(cyclopropylmethyl)-4-(4-(difluoromethoxy)phenyl)-2-(2-methyl-2H-indazol-5-yl)-2,7-dihydro-3H-imidazo[4,5-c]pyridazine-3,6(5H)-dione;
Example 198: 5-(cyclopropylmethyl)-4-(6-cyclopropylpyridin-3-yl)-7-methyl-2-(2-methyl-2H-indazol-5-yl)-2,7-dihydro-3H-imidazo[4,5-c]pyridazine-3,6(5H)-dione;
Example 199: 4-(4-chlorophenyl)-5-(cyclopropylmethyl)-7-methyl-2-(2-methyl-2H-indazol-5-yl)-2,7-dihydro-3H-imidazo[4,5-c]pyridazine-3,6(5H)-dione;
Example 200: 5-(cyclopropylmethyl)-4-(4-cyclopropylphenyl)-7-methyl-2-(2-methyl-2H-indazol-5-yl)-2,7-dihydro-3H-imidazo[4,5-c]pyridazine-3,6(5H)-dione;
Example 201: 5-(cyclopropylmethyl)-4-(4-(methoxy-d3)phenyl)-7-methyl-2-(2-methyl-2H-indazol-5-yl)-2,7-dihydro-3H-imidazo[4,5-c]pyridazine-3,6(5H)-dione;
Example 202: 5-(cyclopropylmethyl)-7-methyl-2-(2-methyl-2H-indazol-5-yl)-4-(6-methylpyridin-3-yl)-2,7-dihydro-3H-imidazo[4,5-c]pyridazine-3,6(5H)-dione;
Example 203: 5-(cyclopropylmethyl)-4-(6-cyclopropylpyridin-3-yl)-2-(2-methyl-2H-indazol-5-yl)-2,7-dihydro-3H-imidazo[4,5-c]pyridazine-3,6(5H)-dione;
Example 204: 5-(cyclopropylmethyl)-4-(4-cyclopropylphenyl)-2-(2-methyl-2H-indazol-5-yl)-2,7-dihydro-3H-imidazo[4,5-c]pyridazine-3,6(5H)-dione;
Example 205: 4-(4-chlorophenyl)-5-(cyclopropylmethyl)-2-(2-methyl-2H-indazol-5-yl)-2,7-dihydro-3H-imidazo[4,5-c]pyridazine-3,6(5H)-dione;
Example 206: 5-(cyclopropylmethyl)-4-(4-(methoxy-d3)phenyl)-2-(2-methyl-2H-indazol-5-yl)-2,7-dihydro-3H-imidazo[4,5-c]pyridazine-3,6(5H)-dione; or
Example 207: 5-(cyclopropylmethyl)-2-(2-methyl-2H-indazol-5-yl)-4-(6-methylpyridin-3-yl)-2,7-dihydro-3H-imidazo[4,5-c]pyridazine-3,6(5H)-dione.

Aspect 61: The compound of Aspect 1, or a pharmaceutically acceptable salt thereof, that is:
Example 208: 1-(cyclopropylmethyl)-7-(4-(difluoromethoxy)phenyl)-5-(2-methyl-2H-indazol-5-yl)-1,5-dihydro-6H-[1,2,3]triazolo[4,5-c]pyridazin-6-one;
Example 209: 1-(cyclopropylmethyl)-7-(4-cyclopropylphenyl)-5-(2-methyl-2H-indazol-5-yl)-1,5-dihydro-6H-[1,2,3]triazolo[4,5-c]pyridazin-6-one; or
Example 210 1-(cyclopropylmethyl)-7-(6-cyclopropylpyridin-3-yl)-5-(2-methyl-2H-indazol-5-yl)-1,5-dihydro-6H-[1,2,3]triazolo[4,5-c]pyridazin-6-one.

Aspect 62: The compound of Aspect 1, or a pharmaceutically acceptable salt thereof, that is:
Example 211: 1-(cyclopropylmethyl)-7-(6-cyclopropylpyridin-3-yl)-3-methyl-5-(2-methyl-2H-indazol-5-yl)-1H,5H,6H-pyrazolo[4,3-c]pyridazin-6-one;
Example 212: 1-(cyclopropylmethyl)-7-(4-(difluoromethoxy)phenyl)-3-methyl-5-(2-methyl-2H-indazol-5-yl)-1,5-dihydro-6H-pyrazolo[4,3-c]pyridazin-6-one;
Example 213: 1-(cyclopropylmethyl)-7-(4-cyclopropylphenyl)-3-methyl-5-(2-methyl-2H-indazol-5-yl)-1,5-dihydro-6H-pyrazolo[4,3-c]pyridazin-6-one;
Example 214: 1-(cyclopropylmethyl)-7-(4-methoxyphenyl)-3-methyl-5-(2-methyl-2H-indazol-5-yl)-1,5-dihydro-6H-pyrazolo[4,3-c]pyridazin-6-one;
Example 215: 7-(4-chlorophenyl)-1-(cyclopropylmethyl)-3-methyl-5-(2-methyl-2H-indazol-5-yl)-1,5-dihydro-6H-pyrazolo[4,3-c]pyridazin-6-one;
Example 216: 1-(cyclopropylmethyl)-7-(4-(methoxy-d3)phenyl)-3-methyl-5-(2-methyl-2H-indazol-5-yl)-1,5-dihydro-6H-pyrazolo[4,3-c]pyridazin-6-one;
Example 217: 1-(cyclopropylmethyl)-3-methyl-5-(2-methyl-2H-indazol-5-yl)-7-(6-methylpyridin-3-yl)-1,5-dihydro-6H-pyrazolo[4,3-c]pyridazin-6-one;
Example 218: 1-(cyclopropylmethyl)-7-(4-(difluoromethoxy)phenyl)-5-(2-methyl-2H-indazol-5-yl)-1,5-dihydro-6H-pyrazolo[4,3-c]pyridazin-6-one;
Example 219: 1-(cyclopropylmethyl)-7-(6-cyclopropylpyridin-3-yl)-5-(2-methyl-2H-indazol-5-yl)-1,5-dihydro-6H-pyrazolo[4,3-c]pyridazin-6-one;
Example 220: 1-(cyclopropylmethyl)-7-(4-(methoxy-d3)phenyl)-5-(2-methyl-2H-indazol-5-yl)-1,5-dihydro-6H-pyrazolo[4,3-c]pyridazin-6-one;
Example 221: 7-(4-chlorophenyl)-1-(cyclopropylmethyl)-5-(2-methyl-2H-indazol-5-yl)-1,5-dihydro-6H-pyrazolo[4,3-c]pyridazin-6-one; or
Example 222: 1-(cyclopropylmethyl)-5-(2-methyl-2H-indazol-5-yl)-7-(6-methylpyridin-3-yl)-1,5-dihydro-6H-pyrazolo[4,3-c]pyridazin-6-one.

Aspect 63: The compound of Aspect 1, or a pharmaceutically acceptable salt thereof, that is:
Example 260: 5-(cyclopropylmethyl)-4-(6-cyclopropylpyridin-3-yl)-7,7-dimethyl-2-(2-methyl-2H-indazol-5-yl)-2,5,6,7-tetrahydro-3H-pyrrolo[3,2-c]pyridazin-3-one.

Aspect 64: The compound of Aspect 1, or a pharmaceutically acceptable salt thereof, that is:
Example 271: 5-(cyclopropylmethyl)-4-(4-(difluoromethoxy)phenyl)-2-(2-methyl-2H-indazol-5-yl)-2,7-dihydro-3H-pyrrolo[2,3-c]pyridazin-3-one; or
Example 272: 5-(cyclopropylmethyl)-4-(4-(difluoromethoxy)phenyl)-7-methyl-2-(2-methyl-2H-indazol-5-yl)-2,7-dihydro-3H-pyrrolo[2,3-c]pyridazin-3-one.

Aspect 1. Aspect 65: The compound of Aspect 1, or a pharmaceutically acceptable salt thereof, that is:

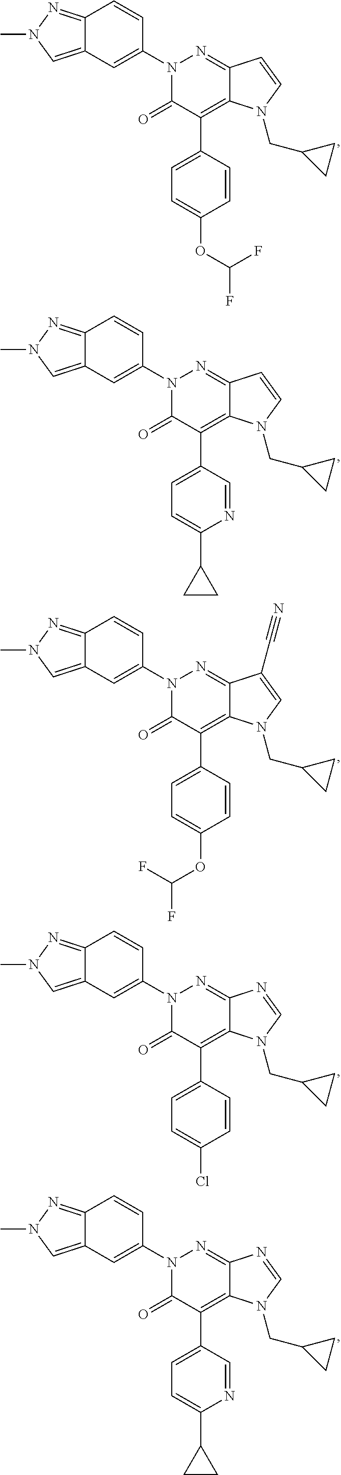

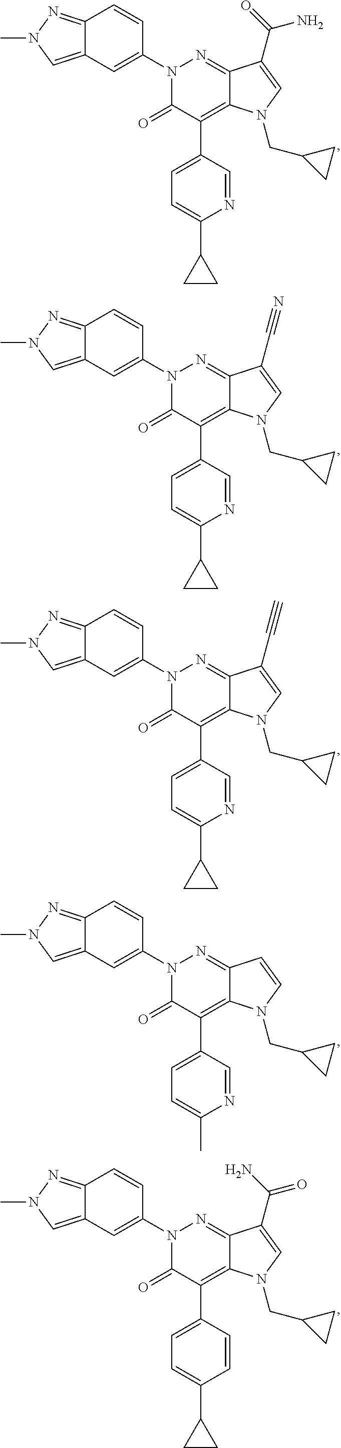 [SMILES: Cn1cc2cc(-n3nc4c(C(N)=O)cn(CC5CC5)c4c(-c4ccc(C5CC5)nc4)c3=O)ccc2n1 ; Cn1cc2cc(-n3nc4c(C#N)cn(CC5CC5)c4c(-c4ccc(C5CC5)nc4)c3=O)ccc2n1 ; C#Cc1cn(CC2CC2)c2c(-c3ccc(C4CC4)nc3)c(=O)n(-c3ccc4nn(C)cc4c3)nc12 ; Cc1ccc(-c2c(=O)n(-c3ccc4nn(C)cc4c3)nc3ccn(CC4CC4)c23)cn1 ; Cn1cc2cc(-n3nc4c(C(N)=O)cn(CC5CC5)c4c(-c4ccc(C5CC5)cc4)c3=O)ccc2n1]

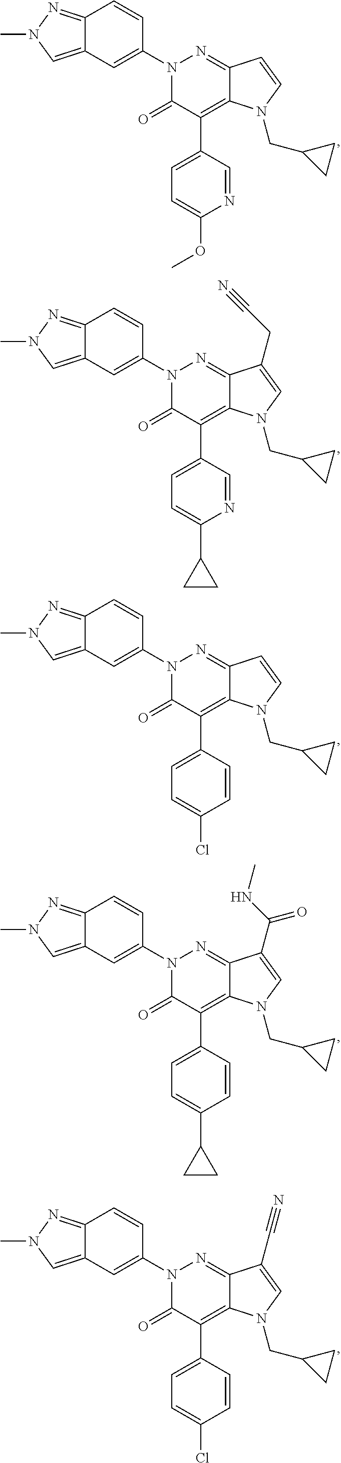

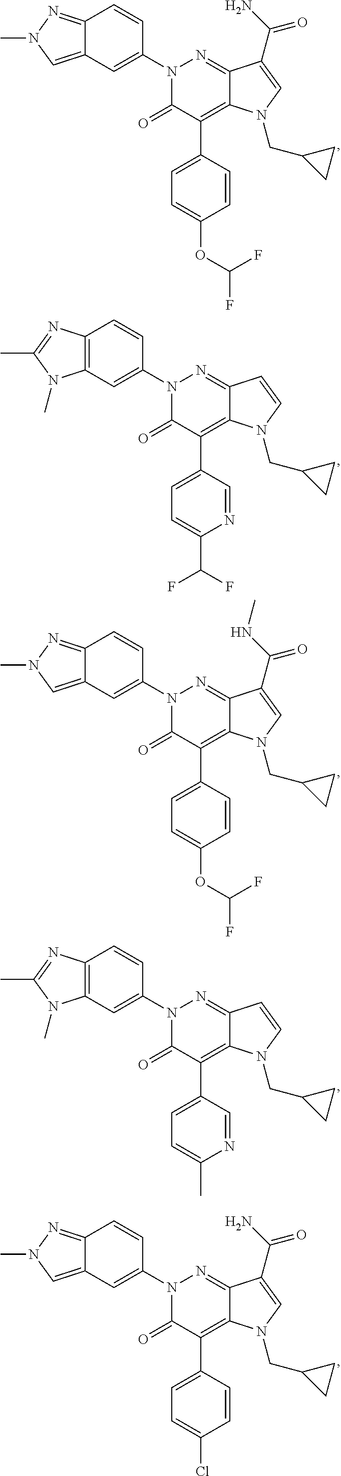

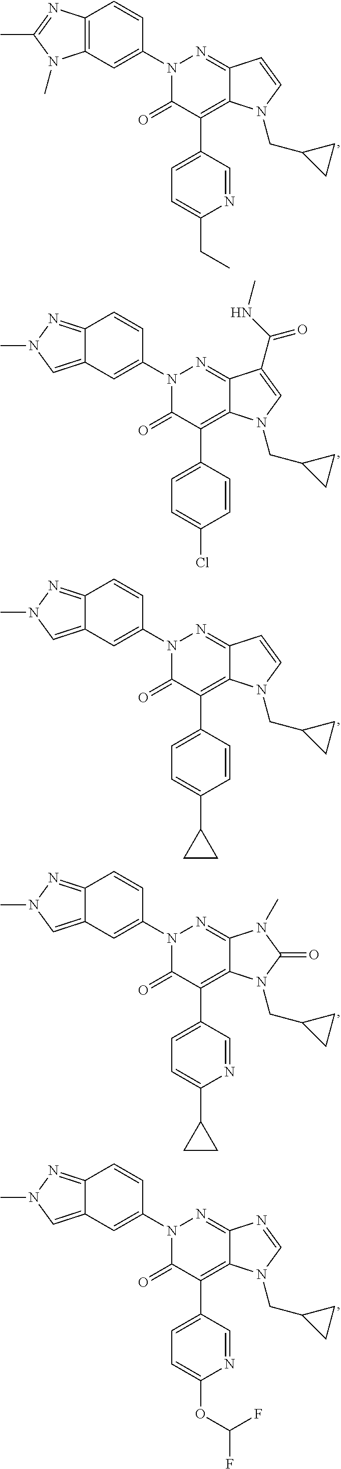

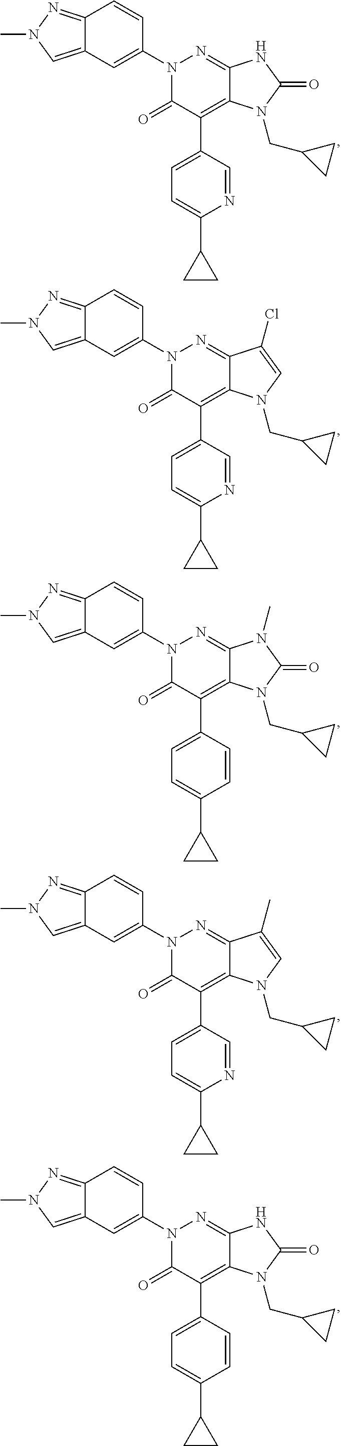 [SMILES: Cn1cc2cc(-n3nc4[nH]c(=O)n(CC5CC5)c4c(-c4ccc(C5CC5)nc4)c3=O)ccc2n1 ; Cn1cc2cc(-n3nc4c(Cl)cn(CC5CC5)c4c(-c4ccc(C5CC5)nc4)c3=O)ccc2n1 ; Cn1cc2cc(-n3nc4c(c(-c5ccc(C6CC6)cc5)c3=O)n(CC3CC3)c(=O)n4C)ccc2n1 ; Cc1cn(CC2CC2)c2c(-c3ccc(C4CC4)nc3)c(=O)n(-c3ccc4nn(C)cc4c3)nc12 ; Cn1cc2cc(-n3nc4[nH]c(=O)n(CC5CC5)c4c(-c4ccc(C5CC5)cc4)c3=O)ccc2n1]

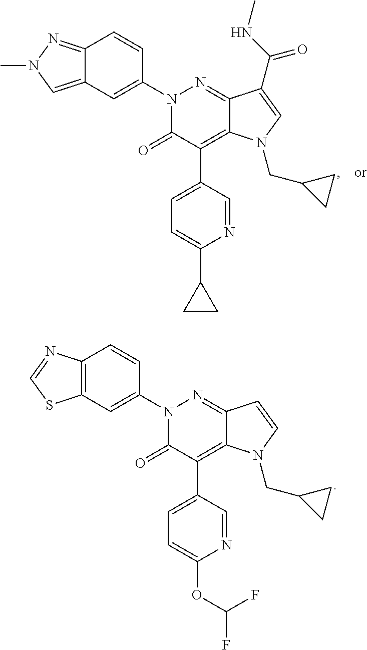 [SMILES: CNC(=O)c1cn(CC2CC2)c2c(-c3ccc(C4CC4)nc3)c(=O)n(-c3ccc4nn(C)cc4c3)nc12 ; O=c1c(-c2ccc(OC(F)F)nc2)c2c(ccn2CC2CC2)nn1-c1ccc2ncsc2c1]

Aspect 66: A method for inhibiting the synthesis of S-adenosyl methionine (SAM) in a subject in need thereof, comprising administering to the subject a SAM-synthesis inhibiting amount of a compound, or a pharmaceutically acceptable salt thereof, of any one of the preceding Aspects.

Aspect 67: A method for treating a disease or condition that responds to the inhibition of MAT2A in a subject in need thereof, comprising administering to the subject an effective amount of a compound, or a pharmaceutically acceptable salt thereof, of any one of Aspects 1 to 65.

Aspect 68: The method of Aspect 66 or 67, wherein said disease or condition is cancer.

Aspect 69: The method of Aspect 68, wherein the cancer is selected from the group consisting of neuroblastoma, rectum carcinoma, colon carcinoma, familiary adenomatous polyposis carcinoma and hereditary non-polyposis colorectal cancer, esophageal carcinoma, labial carcinoma, larynx carcinoma, hypopharynx carcinoma, tong carcinoma, salivary gland carcinoma, gastric carcinoma, adenocarcinoma, medullary thyroidea carcinoma, papillary thyroidea carcinoma, renal carcinoma, kidney parenchym carcinoma, ovarian carcinoma, cervix carcinoma, uterine corpus carcinoma, endometrium carcinoma, chorion carcinoma, pancreatic carcinoma, prostate carcinoma, testis carcinoma, breast carcinoma, urinary carcinoma, melanoma, brain tumors such as glioblastoma, astrocytoma, meningioma, medulloblastoma and peripheral neuroectodermal tumors, Hodgkin lymphoma, non-Hodgkin lymphoma, Burkitt lymphoma, acute lymphatic leukemia (ALL), chronic lymphatic leukemia (CLL), acute myeloid leukemia (AML), chronic myeloid leukemia (CML), adult T-cell leukemia, lymphoma, hepatocellular carcinoma, gall bladder carcinoma, bronchial carcinoma, small cell lung carcinoma, non-small cell lung carcinoma, multiple myeloma, basalioma, teratoma, retinoblastoma, choroidea melanoma, seminoma, rhabdomyo sarcoma, craniopharyngioma, osteosarcoma, chondrosarcoma, myosarcoma, liposarcoma, fibrosarcoma, Ewing sarcoma, and plasmocytoma.

Aspect 70: A method for treating a cancer in a subject suffering therefrom, wherein the cancer is characterized by a reduction or absence of methylthioadenosine phosphorylase (MTAP) gene expression, the absence of the MTAP gene, or reduced function of MTAP protein, the method comprising administering to the subject a therapeutically effective amount of a compound, or a pharmaceutically acceptable salt thereof, of any one of Aspects 1 to 65.

Aspect 71: The method of any one of Aspects 68 to 70, wherein the cancer is MTAP null.

Aspect 72: The method of any one of Aspects 68 to 70, wherein the cancer has a KRAS mutation.

Aspect 73: The method of Aspect 72, wherein the KRAS mutation is an amino acid substitution at residue 12 or 13.

Aspect 74: The method of Aspect 73, wherein said KRAS mutation is G12C, G12R, G12V or G13D.

Aspect 75: The method of any one of Aspects 68 to 70, wherein the cancer has a p53 mutation.

Aspect 76: The method of Aspect 75, wherein the p53 mutation is Y126_splice, K132Q, M133K, R174fs, R175H, R196*, C₂₃₈S, C₂₄₂Y, G245S, R248W, R248Q, I255T, D259V, S261_splice, R267P, R273C, R282W, A159V or R280K.

Aspect 77: A pharmaceutical composition comprising a compound, or a pharmaceutically acceptable salt thereof, of any one of Aspects 1 to 65, and a pharmaceutically acceptable carrier.

Aspect 78: A compound of any one of Aspects 1 to 65, or a pharmaceutically acceptable salt thereof, for inhibiting the synthesis of S-adenosyl methionine (SAM) in a subject.

Aspect 79: A compound of any one of Aspects 1 to 65, or a pharmaceutically acceptable salt thereof, for treating a disease or condition in a subject suffering therefrom, wherein the disease or condition responds to the inhibition of MAT2A.

Aspect 80: A compound of any one of Aspects 1 to 65, or a pharmaceutically acceptable salt thereof, for treating a cancer in a subject suffering therefrom, wherein the cancer is characterized by a reduction or absence of methylthioadenosine phosphorylase (MTAP) expression, the absence of the MTAP gene, or reduced function of MTAP protein.

Aspect 81: A compound of any one of Aspects 1 to 65, or a pharmaceutically acceptable salt thereof, for reducing the plasma concentration of SAM in a subject.

Aspect 82: A method for reducing the plasma concentration of SAM in a subject comprising administering to the subject a SAM-synthesis inhibiting amount of a compound, or a pharmaceutically acceptable salt thereof, of any one of the preceding Aspects.

The following Examples are provided to illustrate concepts described within this application. While each Example is provides specific individual embodiments of composition, methods of preparation and use, none should be considered to limit the more general embodiments described herein. Further, efforts are made in the examples to ensure accuracy with respect to numbers used (e.g. amounts, temperature, etc.) but some experimental error and deviation should be accounted for.

### EXAMPLES

The present application will be more fully understood by reference to the following examples. They should not, however, be construed as limiting the scope of the present application. Reagents and solvents were obtained from commercial sources and used as received.

Abbreviations and terms list:
anhy. anhydrous
aq. aqueous
Inh. inhibition
min minute(s)
mL milliliter
mmol millimole(s)
mol mole(s)
MS mass spectrometry
NMR nuclear magnetic resonance
TLC thin layer chromatography
HPLC high-performance liquid chromatography
RT(r.t) room temperature
Hz hertz
δ chemical shift
coupling constant
s singlet
d doublet
t triplet
q quartet
m multiplet
br broad
qd quartet of doublets
d quin doublet of quintets
dd doublet of doublets
dt doublet of triplets
CHCl₃ chloroform
DCM dichloromethane
DMF dimethyl formamide
Et₂O diethyl ether
EtOH ethyl alcohol
EtOAc ethyl acetate
EA ethyl acetate
MeOH methyl alcohol
MeCN acetonitrile
THF tetrahydrofuran
AcOH acetic acid
HCI hydrochloric acid
H₂SO₄ sulfuric acid
NH₄Cl ammonium chloride
KOH potassium hydroxide
NaOH sodium hydroxide
K₂CO₃ potassium carbonate
Na₂CO₃ sodium carbonate
TFA trifluoroacetic acid
Na₂SO₄ sodium sulfate
NaBH₄ sodium borohydride
NaHCO₃ sodium bicarbonate
LiHMDS lithium hexamethyldisilylamide
NaHMDS sodium hexamethyldisilylamide
LAH lithium aluminum hydride
NaBH₄ sodium borohydride
LDA lithium diisopropylamide
EtsN triethylamine
DMAP 4-(dimethylamino)pyridine
DIPEA N,N-diisopropylethylamine
NH₄OH ammonium hydroxide
EDCI 1-ethyl-3-(3-dimethylaminopropyl)carbodiimide
HOBt 1-hydroxybenzotriazole
HATU O-(7-azabenzotriazol-1-yl)-N,N,N′, N′, -tetra-methyluronium
Xphos 2-Dicyclohexylphosphin-2′, 4′, 6′-triisopropylbiphenyl
BINAP 2,2′-bis(diphenylphosphanyl)-1,1′-binaphthyl

### Examples 101 & Example 102: Synthesis of 5-(cyclopropylmethyl)-4-(4-(difluoromethoxy)phenyl)-2-(2-methyl-2H-indazol-5-yl)-2,5-dihydro-3H-pyrrolo[3,2-c]pyridazin-3-one and 5-(cyclopropylmethyl)-4-(4-(difluoromethoxy)phenyl)-2-(2-methyl-2H-indazol-5-yl)-3-oxo-3,5-dihydro-2H-pyrrolo[3,2-c]pyridazine-7-carbonitrile via General Procedure I (Method A)

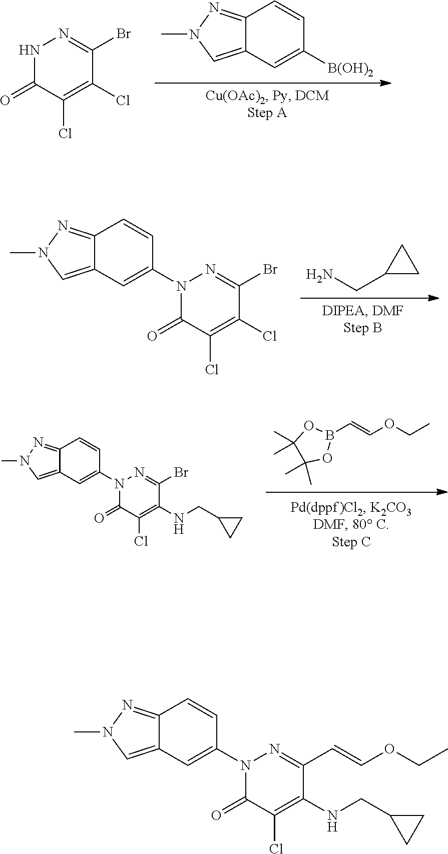

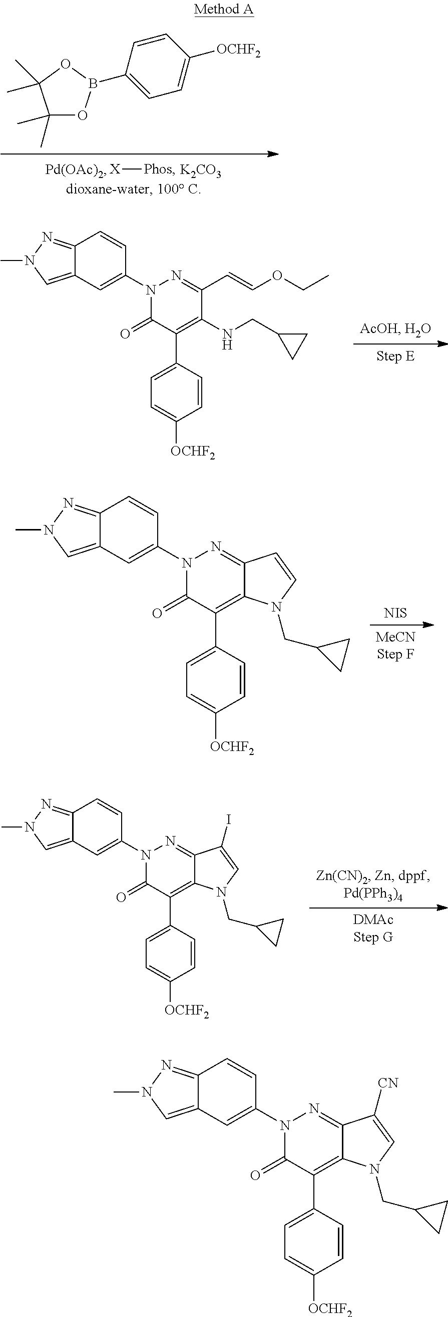

### Step A: 6-bromo-4,5-dichloro-2-(2-methyl-2H-indazol-5-yl)pyridazin-3(2H)-one

To a solution of 6-bromo-4,5-dichloropyridazin-3(2H)-one (14.0 g, 57.4 mmol, 1.0 eq.), (2-methyl-2H-indazol-5-yl)boronic acid (11.1 g, 63.14 mmol, 1.1 eq.) and pyridine (9.1 g, 114.8 mmol, 2.0 eq.) in DMF (250 mL) was added Cu(OAc)₂ (10.4 g, 57.4 mmol, 1.0 eq.), the reaction mixture was stirred at 50° C. under air atmosphere for an additional 8 hrs. After completion, the resulting mixture was poured into NH₄Cl (sat. aq.) (300 mL) at 0° C. A precipitation was formed, the mixture was filtered, the filter cake was washed with H₂O (50 mL) and then dried to give 6-bromo-4,5-dichloro-2-(2-methyl-2H-indazol-5-yl)pyridazin-3(2H)-one (16.2 g, 75%) as a pale brown solid. LC-MS (ESI):m/z 373 [M+H]⁺.

### Step B: 6-bromo-4-chloro-5-((cyclopropylmethyl)amino)-2-(2-methyl-2H-indazol-5-yl)pyridazin-3(2H)-one

To a solution of 6-bromo-4,5-dichloro-2-(2-methyl-2H-indazol-5-yl)pyridazin-3(2H)-one (8.1 g, 21.6 mmol, 1.0 eq.), DIPEA (8.4 g, 65.0 mmol, 3.0 eq.) in DMF (50 mL) was added cyclopropylmethanamine (1.8 g, 25.9 mmol, 1.2 eq.), the reaction mixture was stirred at 85° C. for 5 hrs. The resulting mixture was poured into ice water (100 mL), extracted with EtOAc (50 mL×3), the combined organic layers were washed with brine (50 mL), dried over anhydrous Na₂SO₄ and concentrated under reduced pressure. The resulting residue was purified by column chromatography on silica gel to give 6-bromo-4-chloro-5-((cyclopropylmethyl)amino)-2-(2-methyl-2H-indazol-5-yl)pyridazin-3(2H)-one (3.6 g, 41%) as a yellow solid. LC-MS (ESI):m/z 408 [M+H]⁺.

### Step C: (E)-4-chloro-5-((cyclopropylmethyl)amino)-6-(2-ethoxyvinyl)-2-(2-methyl-2H-indazol-5-yl)pyridazin-3(2H)-one

To a solution of 6-bromo-4-chloro-5-((cyclopropylmethyl)amino)-2-(2-methyl-2H-indazol-5-yl)pyridazin-3(2H)-one (3.6 g, 8.8 mmol, 1.0 eq.), 2-[(E)-2-ethoxyethenyl]-4,4,5,5-tetramethyl-1,3,2-dioxaborolane (2.1 g, 10.5 mmol, 1.2 eq.) in DMF (40 mL) was added K₂CO₃ (3.0 g, 22.0 mmol, 2.5 eq.) and Pd(dppf)Cl₂ (640 mg, 0.88 mmol, 0.1 eq.). The reaction mixture was stirred at 100° C. under N₂ atmosphere for 15 hrs. The resulting mixture diluted with water (40 mL), extracted with EtOAc (50 mL×3), the combined organic layers were washed with brine (40 mL), dried over anhydrous Na₂SO₄ and concentrated under reduced pressure. The resulting residue was purified by column chromatography on silica gel to give (E)-4-chloro-5-((cyclopropylmethyl)amino)-6-(2-ethoxyvinyl)-2-(2-methyl-2H-indazol-5-yl)pyridazin-3(2H)-one (2.6 g, 74%) as a brown oil.

LC-MS (ESI): m/z 400 [M+H]⁺.

### Step D: (E)-5-((cyclopropylmethyl)amino)-4-(4-(difluoromethoxy)phenyl)-6-(2-ethoxyvinyl)-2-(2-methyl-2H-indazol-5-yl)pyridazin-3(2H)-one

To a solution of 2-[4-(difluoromethoxy)phenyl]-4,4,5,5-tetramethyl-1,3,2-dioxaborolane (202 mg, 0.75 mmol, 1.5 eq.), (E)-4-chloro-5-((cyclopropylmethyl)amino)-6-(2-ethoxyvinyl)-2-(2-methyl-2H-indazol-5-yl)pyridazin-3(2H)-one (200 mg, 0.5 mmol, 1.0 eq.) in dioxane (4 mL) and H₂O (1 mL) was added K₂CO₃ (172 mg, 1.25 mmol, 2.5 eq.), X-Phos (47 mg, 0.01 mmol, 0.2 eq.) and Pd(OAc)₂ (12 mg, 0.05 mmol, 0.1 eq.). The reaction mixture was stirred at 100° C. under N₂ atmosphere for 3 hrs. The resulting mixture was filtered through a short pad of Celite®, the filtrate was concentrated under reduced pressure, and the residue was purified by column chromatography on silica gel to give (E)-5-((cyclopropylmethyl)amino)-4-(4-(difluoromethoxy)phenyl)-6-(2-ethoxyvinyl)-2-(2-methyl-2H-indazol-5-yl)pyridazin-3(2H)-one (170 mg, 67%) as a yellow solid. LC-MS (ESI): m/z 508 [M+H]⁺.

### Step E: 5-(cyclopropylmethyl)-4-(4-(difluoromethoxy)phenyl)-2-(2-methyl-2H-indazol-5-yl)-2,5-dihydro-3H-pyrrolo[3,2-c]pyridazin-3-one

A solution of (E)-5-((cyclopropylmethyl)amino)-4-(4-(difluoromethoxy)phenyl)-6-(2-ethoxyvinyl)-2-(2-methyl-2H-indazol-5-yl)pyridazin-3(2H)-one (170 mg, 0.33 mmol, 1.0 eq.) in AcOH (5 mL) was stirred at 80° C. for 4 hrs. The resulting mixture was poured into ice water (40 mL), the aqueous layer was extracted with EtOAc (20 mL×3), the combined organic layers were washed with brine (20 mL), dried over anhydrous Na₂SO₄ and concentrated under reduced pressure. The resulting residue was purified by column chromatography on silica gel to give 5-(cyclopropylmethyl)-4-(4-(difluoromethoxy)phenyl)-2-(2-methyl-2H-indazol-5-yl)-2,5-dihydro-3H-pyrrolo[3,2-c]pyridazin-3-one (Example 101). ¹H NMR (400 MHz, DMSO-d₆) δ (ppm): 8.42 (s, 1H), 7.88 (d, J=1.3 Hz, 1H), 7.69 (d, J=3.8 Hz, 1H), 7.63 (d, J=9.1 Hz, 1H), 7.54 (d, J=8.6 Hz, 2H), 7.37 (dd, J=9.1, 2.0 Hz, 1H), 7.34 (t, J_{HF}=74 Hz, 1H), 7.28 (d, J=8.6 Hz, 2H), 6.43 (d, J=3.8 Hz, 1H), 4.20 (s, 3H), 3.31 (d, J=7.2 Hz, 2H), 0.72-0.65 (m, 1H), 0.45-0.20 (m, 2H), 0.13-0.01 (m, 2H). LC-MS (ESI): m/z 462 [M+H]⁺.

### Step F: 5-(cyclopropylmethyl)-4-(4-(difluoromethoxy)phenyl)-7-iodo-2-(2-methyl-2H-indazol-5-yl)-2,5-dihydro-3H-pyrrolo[3,2-c]pyridazin-3-one

To a solution of 5-(cyclopropylmethyl)-4-(4-(difluoromethoxy)phenyl)-2-(2-methyl-2H-indazol-5-yl)-2,5-dihydro-3H-pyrrolo[3,2-c]pyridazin-3-one (80 mg, 0.17 mmol, 1.0 eq.) in ACN (5 mL) was added NIS (46 mg, 0.21 mmol, 1.2 eq.). The reaction mixture was stirred at 50° C. under N₂ atmosphere for 5 hrs. The resulting mixture was poured into ice water (10 mL), extracted with EtOAc (10 mL×3), the combined organic layers were washed with brine (20 mL), dried over anhydrous Na₂SO₄ and concentrated under reduced pressure. The resulting residue was purified by column chromatography on silica gel to give 5-(cyclopropylmethyl)-4-(4-(difluoromethoxy)phenyl)-7-iodo-2-(2-methyl-2H-indazol-5-yl)-2,5-dihydro-3H-pyrrolo[3,2-c]pyridazin-3-one (55 mg, 55%) as a brown solid. LC-MS (ESI): m/z 588 [M+H]⁺.

### Step G: 5-(cyclopropylmethyl)-4-(4-(difluoromethoxy)phenyl)-2-(2-methyl-2H-indazol-5-yl)-3-oxo-3,5-dihydro-2H-pyrrolo[3,2-c]pyridazine-7-carbonitrile

To a solution of 5-(cyclopropylmethyl)-4-(4-(difluoromethoxy)phenyl)-7-iodo-2-(2-methyl-2H-indazol-5-yl)-2,5-dihydro-3H-pyrrolo[3,2-c]pyridazin-3-one (50 mg, 0.085 mmol, 1.0 eq.), Zn(CN)₂ (38.14 mg, 0.341 mmol, 4.0 eq.), Zn (10 mg, 0.153 mmol, 1.8 eq.) and dppf (10 mg, 0.018 mmol, 0.2 eq.) in DMAc (3 mL) was added Pd(PPh₃)₄ (10 mg, 0.009 mmol, 0.1 eq.), the mixture was stirred at 130° C. under N₂ atmosphere for 1.5 hrs. The resulting mixture was diluted with water (10 mL), extracted with EtOAc (10 mL×3), the combined organic layers were washed with brine (20 mL), dried over anhydrous Na₂SO₄ and concentrated under reduced pressure. The resulting residue was purified by prep-TLC to give 5-(cyclopropylmethyl)-4-(4-(difluoromethoxy)phenyl)-2-(2-methyl-2H-indazol-5-yl)-3-oxo-3,5-dihydro-2H-pyrrolo[3,2-c]pyridazine-7-carbonitrile (Example 102). ¹H NMR (400 MHz, CDCl₃) δ (ppm): 8.10-7.86 (m, 3H), 7.75 (d, J=8.7 Hz, 1H), 7.57-7.42 (m, 4H), 7.20 (s, 1H), 6.58 (t, J_{HF}=74 Hz, 1H), 4.25 (s, 3H), 3.33 (d, J=6.3 Hz, 2H), 0.91-0.76 (m, 1H), 0.67-0.46 (m, 2H), 0.19-0.05 (m, 2H). LC-MS (ESI): m/z 487 [M+H]⁺.

### Examples 103 & Example 104: Synthesis of 5-(cyclopropylmethyl)-2-(2-methyl-2H-indazol-5-yl)-4-(6-methylpyridin-3-yl)-2,5-dihydro-3H-pyrrolo[3,2-c]pyridazin-3-one and 5-(cyclopropylmethyl)-2-(2-methyl-2H-indazol-5-yl)-4-(6-methylpyridin-3-yl)-3-oxo-3,5-dihydro-2H-pyrrolo[3,2-c]pyridazine-7-carbonitrile via General Procedure I (Method B)

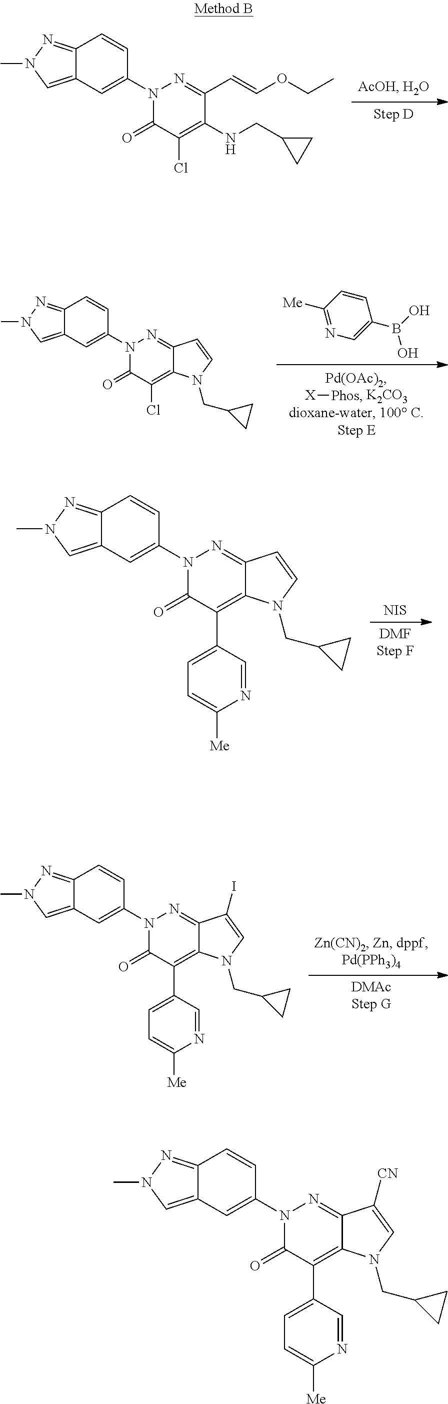

### Step D: 4-chloro-5-(cyclopropylmethyl)-2-(2-methyl-2H-indazol-5-yl)-2,5-dihydro-3H-pyrrolo[3,2-c]pyridazin-3-one

A solution of 4-chloro-5-[(cyclopropylmethyl)amino]-6-[(E)-2-ethoxyethenyl]-2-(2-methyl-2H-indazol-5-yl)-2,3-dihydropyridazin-3-one (2.6 g, 6.50 mmol, 1.0 eq.) in AcOH (30 mL) was stirred at 100° C. for 6 hrs. The resulting mixture was poured into ice water (40 mL), extracted with EtOAc (40 mL×3), the combined organic layers were washed with brine (20 mL), dried over anhydrous Na₂SO₄ and concentrated under reduced pressure. The resulting residue was purified by column chromatography on silica gel to give 4-chloro-5-(cyclopropylmethyl)-2-(2-methyl-2H-indazol-5-yl)-2,5-dihydro-3H-pyrrolo[3,2-c]pyridazin-3-one (1.6 g, 70%) as a yellow solid. LC-MS (ESI): m/z 354 [M+H]⁺.

### Step E: 5-(cyclopropylmethyl)-2-(2-methyl-2H-indazol-5-yl)-4-(6-methylpyridin-3-yl)-2,5-dihydro-3H-pyrrolo[3,2-c]pyridazin-3-one

To a solution of (6-methylpyridin-3-yl)boronic acid (86 mg, 0.633 mmol, 1.4 eq.), 4-chloro-5-(cyclopropylmethyl)-2-(2-methyl-2H-indazol-5-yl)-2,5-dihydro-3H-pyrrolo[3,2-c]pyridazin-3-one (160 mg, 0.452 mmol, 1.0 eq.) in dioxane (4 mL) and H₂O (1 mL) was added K₂CO₃ (156 mg, 1.131 mmol, 2.5 eq.), X-Phos (43 mg, 0.090 mmol, 0.2 eq.) and Pd(OAc)₂ (10 mg, 0.045 mmol, 0.1 eq.). The reaction mixture was stirred at 100° C. under N₂ atmosphere for 3 hrs. The resulting mixture was filtered through a short pad of Celite®, the filtrate was concentrated under reduced pressure, and the residue was purified by column chromatography on silica gel to give 5-(cyclopropylmethyl)-2-(2-methyl-2H-indazol-5-yl)-4-(6-methylpyridin-3-yl)-2,5-dihydro-3H-pyrrolo[3,2-c]pyridazin-3-one (Example 103). ¹H NMR (400 MHz, DMSO-d₆) δ (ppm): 8.53 (d, J=1.8 Hz, 1H), 8.43 (s, 1H), 7.89 (d, J=1.4 Hz, 1H), 7.82 (dd, J=7.9, 2.2 Hz, 1H), 7.71 (d, J=3.8 Hz, 1H), 7.64 (d, J=9.1 Hz, 1H), 7.41-7.33 (m, 2H), 6.45 (d, J=3.8 Hz, 1H), 4.20 (s, 3H), 3.34 (d, J=7.2 Hz, 2H), 2.55 (s, 3H), 0.71-0.62 (m, 1H), 0.37-0.24 (m, 2H), 0.1-0.05 (m, 2H). LC-MS (ESI): m/z 411 [M+H]⁺.

### Step F: 5-(cyclopropylmethyl)-7-iodo-2-(2-methyl-2H-indazol-5-yl)-4-(6-methylpyridin-3-yl)-2,5-dihydro-3H-pyrrolo[3,2-c]pyridazin-3-one

To a solution of 5-(cyclopropylmethyl)-2-(2-methyl-2H-indazol-5-yl)-4-(6-methylpyridin-3-yl)-2,5-dihydro-3H-pyrrolo[3,2-c]pyridazin-3-one (83 mg, 0.20 mmol, 1.0 eq.) in anhydrous DMF (3 mL) was added NIS (54 mg, 0.24 mmol, 1.2 eq.), the reaction mixture was stirred at 50° C. under N₂ atmosphere for 5 hrs. The resulting mixture was poured into ice water (10 mL), extracted with ethyl acetate (10 mL×3), the combined organic layers were washed with brine (10 mL), dried over anhydrous Na₂SO₄ and concentrated under reduced pressure. The resulting residue was purified by column chromatography on silica gel to give 5-(cyclopropylmethyl)-7-iodo-2-(2-methyl-2H-indazol-5-yl)-4-(6-methylpyridin-3-yl)-2,5-dihydro-3H-pyrrolo[3,2-c]pyridazin-3-one (58 mg, 53%) as a brown solid. LC-MS (ESI): m/z 537 [M+H]⁺.

### Step G: 5-(cyclopropylmethyl)-2-(2-methyl-2H-indazol-5-yl)-4-(6-methylpyridin-3-yl)-3-oxo-3,5-dihydro-2H-pyrrolo[3,2-c]pyridazine-7-carbonitrile

To a solution of 5-(cyclopropylmethyl)-7-iodo-2-(2-methyl-2H-indazol-5-yl)-4-(6-methylpyridin-3-yl)-2,5-dihydro-3H-pyrrolo[3,2-c]pyridazin-3-one (58 mg, 0.11 mmol, 1.0 eq.), Zn(CN)₂ (48 mg, 0.43 mmol, 4.0 eq.), Zn (10 mg, 0.15 mmol, 1.4 eq.), dppf (10 mg, 0.018 mmol, 0.2 eq.) in DMAc (3 mL) was added Pd(PPh₃)₄ (12 mg, 0.011 mmol, 0.1 eq.). The mixture was stirred at 130° C. under N₂ atmosphere for 1.5 hrs. The resulting mixture was diluted with water (10 mL), extracted with EtOAc (10 mL×3), the combined organic layers were washed with brine (20 mL), dried over anhydrous Na₂SO₄ and concentrated under reduced pressure. The resulting residue was purified by prep-TLC to give 5-(cyclopropylmethyl)-2-(2-methyl-2H-indazol-5-yl)-4-(6-methylpyridin-3-yl)-3-oxo-3,5-dihydro-2H-pyrrolo[3,2-c]pyridazine-7-carbonitrile (Example 104). ¹H NMR (400 MHz, DMSO-d₆) δ (ppm): 8.72 (s, 1H), 8.56 (d, J=1.6 Hz, 1H), 8.47 (s, 1H), 7.95 (d, J=1.2 Hz, 1H), 7.85 (dd, J=8.0, 2.4 Hz, 1H), 7.68 (d, J=9.2 Hz, 1H), 7.50-7.31 (m, 2H), 4.22 (s, 3H), 3.39 (d, J=7.2 Hz, 2H), 2.56 (s, 3H), 0.78-0.67 (m, 1H), 0.43-0.27 (m, 2H), 0.22-0.04 (m, 2H). LC-MS (ESI): m/z 436 [M+H]⁺.

The procedure set forth above for General Procedure I (Method A) was used to synthesize the following compounds by using appropriate starting materials:

| Example | Structure | Characterization |
|---|---|---|
| 105 | 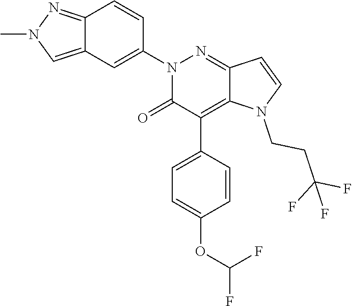 [SMILES: Cn1cc2cc(-n3nc4ccn(CCC(F)(F)F)c4c(-c4ccc(OC(F)F)cc4)c3=O)ccc2n1] | LC-MS: m/z 504 (M + H)⁺. ¹H NMR (400 MHz, DMSO- d₆) δ: 8.42 (s, 1H), 7.88 (d, J = 1.5 Hz, 1H), 7.69 (d, J = 3.8 Hz, 1H), 7.64 (d, J = 9.2 Hz, 1H), 7.58 (d, J = 8.6 Hz, 2H), 7.36 (dd, J = 9.2, 2.0 Hz, 1H), 7.31 (t, J_{HF} = 74 Hz, 1H), 7.28 (d, J = 8.4 Hz, 2H), 6.47 (d, J = 3.8 Hz, 1H), 4.20 (s, 3H), 3.94-3.64 (m, 2H), 2.41-2.18 (m, 2H). |
| | 4-(4-(difluoromethoxy)phenyl)-2-(2-methyl-2H-indazol-5- | |
| | yl)-5-(3,3,3-trifluoropropyl)-2,5-dihydro-3H-pyrrolo[3,2- | |
| | c]pyridazin-3-one | |
| 106 | 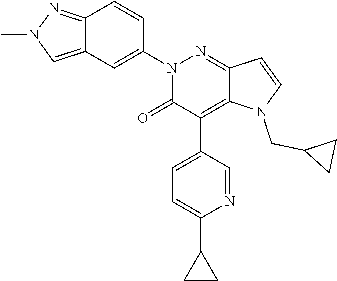 [SMILES: Cn1cc2cc(-n3nc4ccn(CC5CC5)c4c(-c4ccc(C5CC5)nc4)c3=O)ccc2n1] | LC-MS: m/z 437 (M + H)⁺. ¹H NMR (400 MHz, DMSO- d₆) δ: 8.39 (d, J = 1.9 Hz, 1H), 8.34 (s, 1H), 7.81 (d, J = 1.4 Hz, 1H), 7.69 (dd, J = 8.0, 2.2 Hz, 1H), 7.62 (d, J = 3.8 Hz, 1H), 7.55 (d, J = 9.2 Hz, 1H), 7.33 (d, J = 8.0 Hz, 1H), 7.29 (dd, J = 9.2, 2.0 Hz, 1H), 6.36 (d, J = 3.8 Hz, 1H), 4.12 (s, 3H), 3.23 (d, J = 6.7 Hz, 2H), 2.11-2.07 (m, 1H), 1.02-0.86 (m, 4H), 0.61-0.52 (m, 1H), 0.25-0.19 (m, 2H), −0.01-0.05 (m, 2H). |
| | 5-(cyclopropylmethyl)-4-(6-cyclopropylpyridin-3-yl)-2- | |
| | (2-methyl-2H-indazol-5-yl)-2,5-dihydro-3H-pyrrolo[3,2- | |
| | c]pyridazin-3-one | |
| 107 | 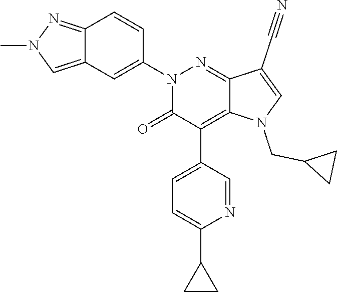 [SMILES: Cn1cc2cc(-n3nc4c(C#N)cn(CC5CC5)c4c(-c4ccc(C5CC5)nc4)c3=O)ccc2n1] | LC-MS: m/z 462 (M + H)⁺. ¹H NMR (400 MHz, DMSO- d₆) δ: 8.71 (s, 1H), 8.50 (d, J = 1.9 Hz, 1H), 8.46 (s, 1H), 7.94 (d, J = 1.4 Hz, 1H), 7.80 (dd, J = 8.0, 2.2 Hz, 1H), 7.68 (d, J = 9.2 Hz, 1H), 7.43 (d, J = 7.7 Hz, 1H), 7.38 (dd, J = 9.2, 2.0 Hz, 1H), 4.21 (s, 3H), 3.38 (d, J = 7.2 Hz, 2H), 2.26-2.12 (m, 1H), 1.04-0.94 (m, 4H), 0.74- 0.70 (m, 1H), 0.41-0.32 (m, 2H), 0.18-0.11 (m, 2H) |
| | 5-(cyclopropylmethyl)-4-(6-cyclopropylpyridin-3-yl)-2- | |
| | (2-methyl-2H-indazol-5-yl)-3-oxo-3,5-dihydro-2H- | |
| | pyrrolo[3,2-c]pyridazine-7-carbonitrile | |
| 108 | 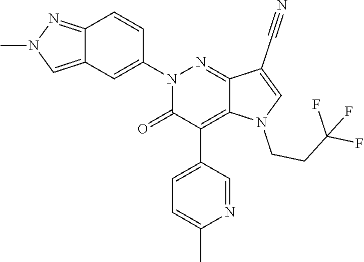 [SMILES: Cc1ccc(-c2c(=O)n(-c3ccc4nn(C)cc4c3)nc3c(C#N)cn(CCC(F)(F)F)c23)cn1] | LC-MS: m/z 478 (M + H)⁺. ¹H NMR (400 MHz, DMSO- d₆) δ: 8.68 (s, 1H), 8.58 (d, J = 2.0 Hz, 1H), 8.47 (s, 1H), 7.94 (d, J = 1.6 Hz, 1H), 7.88 (dd, J = 8.0, 2.4 Hz, 1H), 7.68 (d, J = 9.2 Hz, 1H), 7.44-7.34 (m, 2H), 4.21 (s, 3H), 3.87-3.84 (m, 2H), 2.53 (s, 3H), 2.41- 2.34 (m, 2H). |
| | 2-(2-methyl-2H-indazol-5-yl)-4-(6-methylpyridin-3-yl)-3-oxo- | |
| | 5-(3,3,3-trifluoropropyl)-3,5-dihydro-2H-pyrrolo[3,2- | |
| | c]pyridazine-7-carbonitrile | |
| 109 | 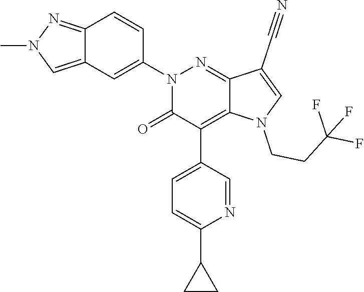 [SMILES: Cn1cc2cc(-n3nc4c(C#N)cn(CCC(F)(F)F)c4c(-c4ccc(C5CC5)nc4)c3=O)ccc2n1] | LC-MS: m/z 504 (M + H)⁺. ¹H NMR (400 MHz, DMSO- d₆) δ: 8.68 (s, 1H), 8.52 (d, J = 2.0 Hz, 1H), 8.47 (s, 1H), 7.94 (d, J = 1.6 Hz, 1H), 7.82 (dd, J = 8.0, 2.0 Hz, 1H), 7.68 (d, J = 9.2 Hz, 1H), 7.44 (d, J = 8.0 Hz, 1H), 7.37 (dd, J = 9.2, 2.0 Hz, 1H), 4.21 (s, 3H), 3.89- 3.75 (m, 2H), 2.44-2.30 (m, 2H), 2.25-2.15 (m, 1H), 0.94- 0.79 (m, 4H). |
| | 4-(6-cyclopropylpyridin-3-yl)-2-(2-methyl-2H-indazol-5-yl)-3- | |
| | oxo-5-(3,3,3-trifluoropropyl)-3,5-dihydro-2H-pyrrolo[3,2- | |
| | c]pyridazine-7-carbonitrile | |

The procedure set forth above for General Procedure I (Method B) was used to synthesize the following compounds by using appropriate starting materials:

| Example | Structure | Characterization |
|---|---|---|
| 110 | 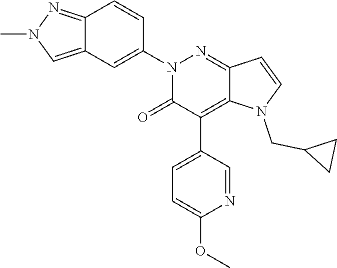 [SMILES: COc1ccc(-c2c(=O)n(-c3ccc4nn(C)cc4c3)nc3ccn(CC4CC4)c23)cn1] | LC-MS: m/z 427 (M + H)⁺. ¹H NMR (400 MHz, DMSO- d₆) δ: 8.43 (s, 1H), 8.26 (d, J = 2.4 Hz, 1H), 7.89 (dd, J = 1.9, 0.7 Hz, 1H), 7.85 (dd, J = 8.5, 2.4 Hz, 1H), 7.70 (d, J = 3.8 Hz, 1H), 7.64 (d, J = 9.1 Hz, 1H), 7.37 (dd, J = 9.1, 2.0 Hz, 1H), 6.94 (dd, J = 8.5, 0.6 Hz, 1H), 6.44 (d, J = 3.8 Hz, 1H), 4.20 (s, 3H), 3.91 (s, 3H), 3.38 (d, J = 6.7 Hz, 2H), 0.72- 0.66 (m, 1H), 0.40-0.26 (m, 2H), 0.12-0.08 (m, 2H). |
| | 5-(cyclopropylmethyl)-4-(6-methoxypyridin-3-yl)-2-(2- | |
| | methyl-2H-indazol-5-yl)-2,5-dihydro-3H-pyrrolo[3,2- | |
| | c]pyridazin-3-one | |
| 111 | 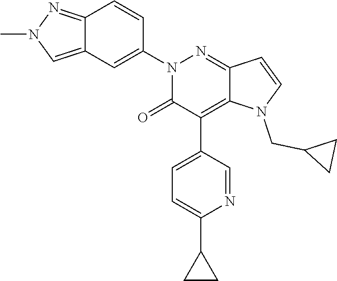 | LC-MS: m/z 452 (M + H)⁺. ¹H NMR (400 MHz, DMSO- d₆) δ: 8.71 (s, 1H), 8.46 (s, 1H), 8.30 (d, J = 2.3 Hz, 1H), 7.94 (d, J = 1.9 Hz, 1H), 7.88 (dd, J = 8.5, 2.4 Hz, 1H), 7.68 (d, J = 9.1 Hz, 1H), 7.38 (dd, J = 9.1, 2.0 Hz, 1H), 6.96 (d, J = 8.6 Hz, 1H), 4.21 (s, 3H), 3.92 (s, 3H), 3.43 (d, J = 7.2 Hz, 2H), 0.78-0.69 (m, 1H), 0.42-0.36 (m, 2H), 0.17-0.12 (m, 2H). |
| | 5-(cyclopropylmethyl)-4-(6-methoxypyridin-3-yl)-2-(2- | |
| | methyl-2H-indazol-5-yl)-3-oxo-3,5-dihydro-2H- | |
| | pyrrolo[3,2-c]pyridazine-7-carbonitrile | |
| 112 | 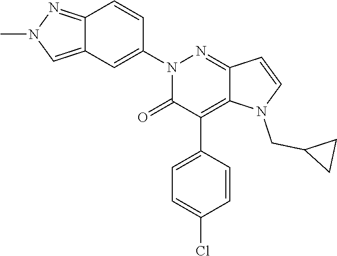 [SMILES: Cn1cc2cc(-n3nc4ccn(CC5CC5)c4c(-c4ccc(Cl)cc4)c3=O)ccc2n1] | LC-MS: m/z 430 (M + H)⁺. ¹H NMR (400 MHz, DMSO- d₆) δ: 8.42 (s, 1H), 7.88 (d, J = 1.3 Hz, 1H), 7.69 (d, J = 3.8 Hz, 1H), 7.63 (d, J = 9.1 Hz, 1H), 7.58-7.44 (m, 4H), 7.37 (dd, J = 9.1, 2.0 Hz, 1H), 6.44 (d, J = 3.8 Hz, 1H), 4.20 (s, 3H), 3.31 (d, J = 7.1 Hz, 2H), 0.70-0.64 (m, 1H), 0.34-0.24 (m, 2H), 0.96-0.05 (m, 2H) |
| | 4-(4-chlorophenyl)-5-(cyclopropylmethyl)-2-(2-methyl- | |
| | 2H-indazol-5-yl)-2,5-dihydro-3H-pyrrolo[3,2-c]pyridazin- | |
| | 3-one | |
| 113 | 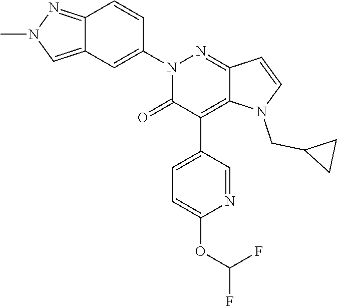 [SMILES: Cn1cc2cc(-n3nc4ccn(CC5CC5)c4c(-c4ccc(OC(F)F)nc4)c3=O)ccc2n1] | LC-MS: m/z 463 (M + H)⁺. ¹H NMR (400 MHz, DMSO- d₆) δ: 8.42 (d, J = 8.9 Hz, 1H), 8.40 (d, J = 1.9 Hz, 1H), 8.09 (dd, J = 8.4, 2.4 Hz, 1H), 7.91- 7.87 (m, 1H), 7.80 (t, J_{HF} = 74 Hz, 1H), 7.72 (d, J = 3.8 Hz, 1H), 7.64 (d, J = 9.1 Hz, 1H), 7.37 (dd, J = 9.1, 2.0 Hz, 1H), 7.22 (d, J = 8.5 Hz, 1H), 6.47 (d, J = 3.8 Hz, 1H), 4.20 (s, 3H), 3.32 (d, J = 7.1 Hz, 2H), 0.74-0.59 (m, 1H), 0.41- 0.23 (m, 2H), 0.13-0.09 (q, J = 4.9 Hz, 2H). |
| | 5-(cyclopropylmethyl)-4-(6-(difluoromethoxy)pyridin-3- | |
| | yl)-2-(2-methyl-2H-indazol-5-yl)-2,5-dihydro-3H- | |
| | pyrrolo[3,2-c]pyridazin-3-one | |
| 114 | 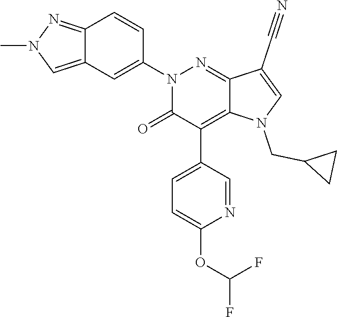 [SMILES: Cn1cc2cc(-n3nc4c(C#N)cn(CC5CC5)c4c(-c4ccc(OC(F)F)nc4)c3=O)ccc2n1] | LC-MS: m/z 488 (M + H)⁺. ¹H NMR (400 MHz, CD₃OD) δ: 8.48 (s, 1H), 8.33 (s, 2H), 8.16-7.33 (m, 5H), 7.13 (s, 1H), 4.25 (s, 3H), 3.41 (d, J = 7.2 Hz, 2H), 0.93-0.75 (m, 1H), 0.62-0.43 (m, 2H), 0.35- 0.06 (m, 2H). |
| | 5-(cyclopropylmethyl)-4-(6-(difluoromethoxy)pyridin-3- | |
| | yl)-2-(2-methyl-2H-indazol-5-yl)-3-oxo-3,5-dihydro-2H- | |
| | pyrrolo[3,2-c]pyridazine-7-carbonitrile | |
| 115 | 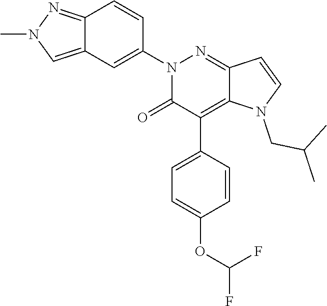 [SMILES: CC(C)Cn1ccc2nn(-c3ccc4nn(C)cc4c3)c(=O)c(-c3ccc(OC(F)F)cc3)c21] | LC-MS: m/z 464 (M + H)⁺. ¹H NMR (400 MHz, DMSO- d₆) δ: 8.42 (s, 1H), 7.89 (d, J = 1.4 Hz, 1H), 7.64-7.61 (m, 2H), 7.52 (d, J = 8.7 Hz, 2H), 7.37 (dd, J = 9.2, 2.0 Hz, 1H), 7.33 (t, J_{HF} = 74 Hz, 1H), 7.29 (d, J = 8.5 Hz, 2H), 6.41 (d, J = 3.7 Hz, 1H), 4.20 (s, 3H), 3.27 (s, 2H), 1.40-1.28 (m, 1H), 0.46 (d, J = 6.6 Hz, 6H). |
| | 4-(4-(difluoromethoxy)phenyl)-5-isobutyl-2-(2-methyl- | |
| | 2H-indazol-5-yl)-2,5-dihydro-3H-pyrrolo[3,2- | |
| | c]pyridazin-3-one | |
| 116 | 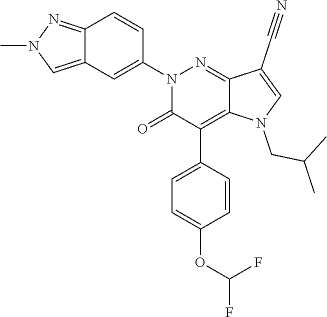 [SMILES: CC(C)Cn1cc(C#N)c2nn(-c3ccc4nn(C)cc4c3)c(=O)c(-c3ccc(OC(F)F)cc3)c21] | LC-MS: m/z 489 (M + H)⁺. ¹H NMR (400 MHz, DMSO- d₆) δ: 8.62 (s, 1H), 8.46 (s, 1H), 7.94 (s, 1H), 7.67 (d, J = 9.1 Hz, 1H), 7.56 (d, J = 8.6 Hz, 2H), 7.39 (dd, J = 9.2, 1.9 Hz, 1H), 7.33 (t, J_{HF} = 74 Hz, 1H), 7.32 (d, J = 8.6 Hz, 2H), 4.21 (s, 3H), 3.39 (d, J = 7.3 Hz, 2H), 1.39-1.27 (m, 1H), 0.47 (d, J = 6.6 Hz, 6H). |
| | 4-(4-(difluoromethoxy)phenyl)-5-isobutyl-2-(2-methyl- | |
| | 2H-indazol-5-yl)-3-oxo-3,5-dihydro-2H-pyrrolo[3,2- | |
| | c]pyridazine-7-carbonitrile | |
| 117 (From Step F) | 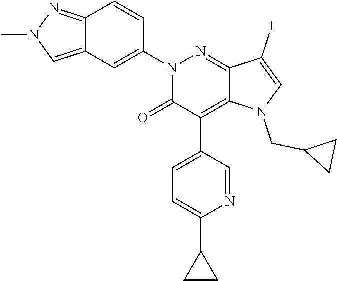 [SMILES: Cn1cc2cc(-n3nc4c(I)cn(CC5CC5)c4c(-c4ccc(C5CC5)nc4)c3=O)ccc2n1] | LC-MS: m/z 563 (M + H)⁺. ¹H NMR (400 MHz, DMSO- d₆) δ: 8.38 (s, 1H), 8.37 (d, J = 2.8 Hz, 1H),7.83 (s, 1H), 7.81 (d, J = 2.0 Hz, 1H), 7.68 (dd, J = 8.0, 2.0 Hz, 1H), 7.58 (d, J = 9.2 Hz, 1H), 7.33 (d, J = 8.0 Hz, 1H), 7.28 (dd, J = 9.2, 2.0 Hz, 1H), 4.13 (s, 3H), 3.21 (d, J = 7.2 Hz, 2H), 2.13-2.05 (m, 1H), 0.95-0.87 (m, 4H), 0.66-0.53 (m, 1H), 0.27-0.18 (m, 2H), 0.03-−0.05 (m, 2H). |
| | 5-(cyclopropylmethyl)-4-(6-cyclopropylpyridin-3-yl)-7- | |
| | iodo-2-(2-methyl-2H-indazol-5-yl)-2,5-dihydro-3H- | |
| | pyrrolo[3,2-c]pyridazin-3-one | |

### Examples 118 & Example 119: Synthesis of 5-(cyclopropylmethyl)-2-(2-methyl-2H-indazol-5-yl)-4-(6-(trifluoromethyl)pyridin-3-yl)-2,5-dihydro-3H-pyrrolo[3,2-c]pyridazin-3-one and 5-(cyclopropylmethyl)-2-(2-methyl-2H-indazol-5-yl)-3-oxo-4-(6-(trifluoromethyl)pyridin-3-yl)-3,5-dihydro-2H-pyrrolo[3,2-c]pyridazine-7-carbonitrile via General Procedure II (Method A)

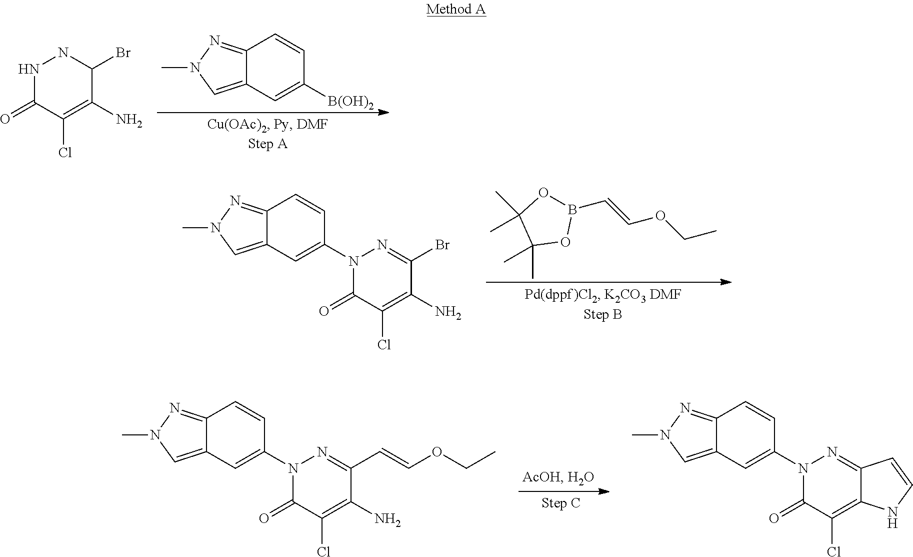

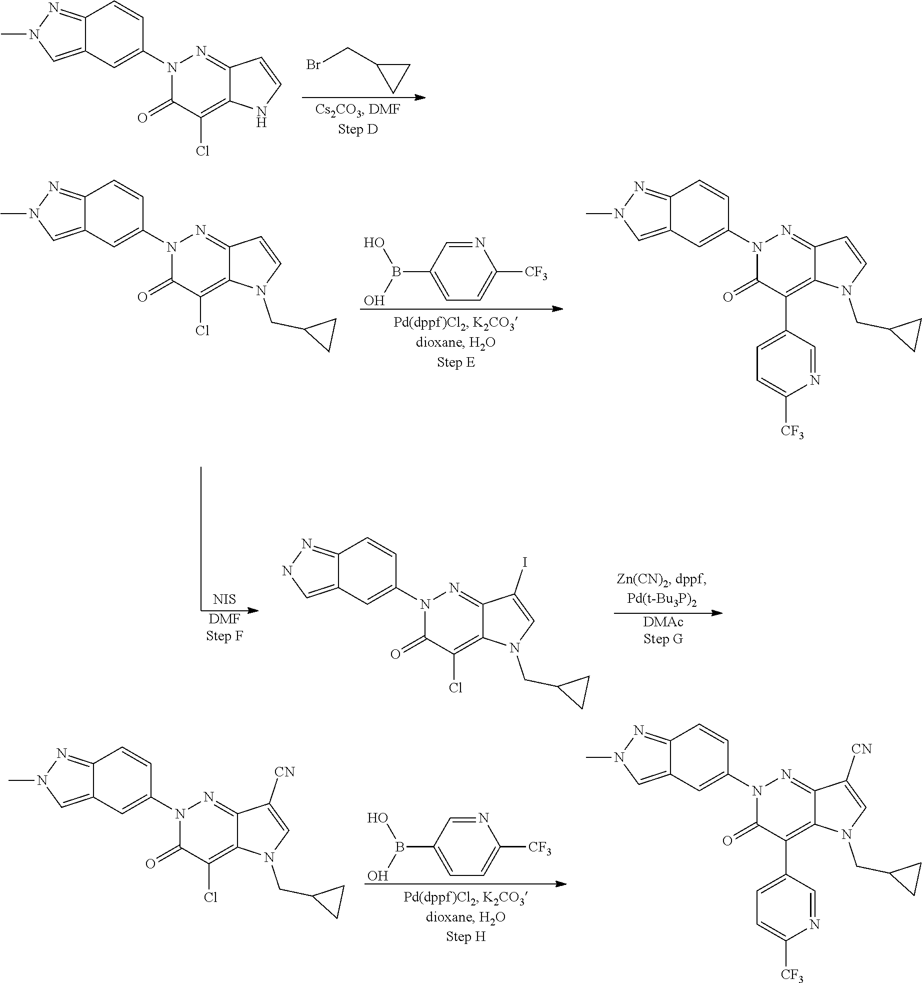

### Step A: 5-amino-6-bromo-4-chloro-2-(2-methyl-2H-indazol-5-yl)pyridazin-3(2H)-one

To a solution of 5-amino-6-bromo-4-chloropyridazin-3(2H)-one (4.5 g, 20.0 mmol, 1.0 eq.), (2-methyl-2H-indazol-5-yl)boronic acid (4.2 g, 24.0 mmol, 1.2 eq.), pyridine (3.2 g, 40.0 mmol, 2.0 eq.) in DMF (60 mL) was added Cu(OAc)₂ (4.4 g, 24.0 mmol, 1.0 eq.), the reaction mixture was stirred at 50° C. open to air for 8 hrs. The resulting mixture was poured into NH₄Cl (sat. aq.) (100 mL) at 0° C., a precipitation was formed, the mixture was filtered, the filter cake was washed with H₂O (50 mL), then dried to give 5-amino-6-bromo-4-chloro-2-(2-methyl-2H-indazol-5-yl)pyridazin-3(2H)-one (5.4 g, 75%) as a light brown solid. LC-MS (ESI): m/z 353 [M+H]⁺.

### Step B: (E)-5-amino-4-chloro-6-(2-ethoxyvinyl)-2-(2-methyl-2H-indazol-5-yl)pyridazin-3(2H)-one

To a solution of 5-amino-6-bromo-4-chloro-2-(2-methyl-2H-indazol-5-yl)pyridazin-3(2H)-one (710 mg, 2.00 mmol, 1.0 eq.) and (E)-2-(2-ethoxyvinyl)-4,4,5,5-tetramethyl-1,3,2-dioxaborolane (475 mg, 2.40 mmol, 1.2 eq.) in DMF (10 mL) was added K₂CO₃ (691 mg, 5.01 mmol, 2.5 eq.) and Pd(dppf)Cl₂ (146 mg, 0.20 mmol, 0.1 eq.), the reaction mixture was stirred at 100° C. under N₂ atmosphere for 5 hrs. The resulting mixture diluted with water (30 mL), extracted with EtOAc (20 mL×3), the combined organic layers were washed with brine (20 mL), dried over anhydrous Na₂SO₄ and concentrated under reduced pressure. The resulting residue was purified by column chromatography on silica gel to give (E)-5-amino-4-chloro-6-(2-ethoxyvinyl)-2-(2-methyl-2H-indazol-5-yl)pyridazin-3(2H)-one (560 mg, 81%) as a brown oil. LC-MS (ESI): m/z 346 [M+H]⁺.

### Step C: 4-chloro-2-(2-methyl-2H-indazol-5-yl)-2,5-dihydro-3H-pyrrolo[3,2-c]pyridazin-3-one

A solution of 5-amino-4-chloro-6-[(E)-2-ethoxyethenyl]-2-(2-methyl-2H-indazol-5-yl)-2,3-dihydropyridazin-3-one (560 mg, 1.62 mmol, 1.0 eq.) in AcOH (8 mL) was stirred at 100° C. for 6 hrs. The resulting mixture was poured into ice water (30 mL), a precipitation was formed, the mixture was filtered, the filter cake was washed with H₂O (20 mL) and then dried to give 4-chloro-2-(2-methyl-2H-indazol-5-yl)-2,5-dihydro-3H-pyrrolo[3,2-c]pyridazin-3-one (350 mg, 72%) as a yellow solid. LC-MS (ESI): m/z 300 [M+H]⁺.

### Step D: 4-chloro-5-(cyclopropylmethyl)-2-(2-methyl-2H-indazol-5-yl)-2,5-dihydro-3H-pyrrolo[3,2-c]pyridazin-3-one

To a solution of 4-chloro-2-(2-methyl-2H-indazol-5-yl)-2H,3H,5H-pyrrolo[3,2-c]pyridazin-3-one (350 mg, 0.93 mmol, 1.0 eq.) in DMF (4 mL) was added Cs₂CO₃ (760 mg, 2.33 mmol, 2.5 eq.) and (bromomethyl)cyclopropane (188 mg, 1.40 mmol, 1.5 eq.), the reaction mixture was stirred at 50° C. for 6 hrs. The resulting mixture was poured into ice water (15 mL), extracted with EtOAc (15 mL×3), the combined organic layers were washed with brine (10 mL), dried over anhydrous Na₂SO₄ and concentrated under reduced pressure. The resulting residue was purified by column chromatography on silica gel to give 4-chloro-5-(cyclopropylmethyl)-2-(2-methyl-2H-indazol-5-yl)-2H,3H,5H-pyrrolo[3,2-c]pyridazin-3-one (230 mg, 70%) as a yellow solid. LC-MS (ESI): m/z 354 [M+H]⁺.

### Step E: 5-(cyclopropylmethyl)-2-(2-methyl-2H-indazol-5-yl)-4-(6-(trifluoromethyl)pyridin-3-yl)-2,5-dihydro-3H-pyrrolo[3,2-c]pyridazin-3-one

To a solution of 4-chloro-5-(cyclopropylmethyl)-2-(2-methyl-2H-indazol-5-yl)-2,5-dihydro-3H-pyrrolo[3,2-c]pyridazin-3-one (40 mg, 113 μmol, 1.0 eq.), (6-(trifluoromethyl)pyridin-3-yl)boronic acid (28 mg, 147 μmol, 1.3 eq.) and Pd(dppf)Cl₂ (8 mg, 11.3 μmol, 0.1 eq.) in dioxane (1 mL) was added a solution of K₂CO₃ (31 mg, 226 μmol, 2.0 eq.) in H₂O (0.3 mL), the reaction mixture was stirred at 100° C. under N₂ atmosphere for 3 hrs. The resulting mixture was filtered through a short pad of Celite®, the filtrate was concentrated under reduced pressure, and the residue was purified by prep-TLC to give 5-(cyclopropylmethyl)-2-(2-methyl-2H-indazol-5-yl)-4-(6-(trifluoromethyl)pyridin-3-yl)-2,5-dihydro-3H-pyrrolo[3,2-c]pyridazin-3-one. (Example 118). ¹H NMR (400 MHz, CDCl₃) δ (ppm): 8.81 (s, 1H), 8.15 (s, 1H), 7.95 (s, 2H), 7.79 (d, J=8.6 Hz, 2H), 7.50 (d, J=9.3 Hz, 1H), 7.37 (d, J=3.7 Hz, 1H), 6.49 (d, J=3.7 Hz, 1H), 4.25 (s, 3H), 3.31 (d, J=6.8 Hz, 2H), 0.80-72 (m, 1H), 0.55-0.48 (m, 2H), 0.12-0.08 (m, 2H). LC-MS (ESI): m/z 465 [M+H]⁺.

### Step F: 4-chloro-5-(cyclopropylmethyl)-7-iodo-2-(2-methyl-2H-indazol-5-yl)-2,5-dihydro-3H-pyrrolo[3,2-c]pyridazin-3-one

To a solution of 4-chloro-5-(cyclopropylmethyl)-2-(2-methyl-2H-indazol-5-yl)-2H,3H,5H-pyrrolo[3,2-c]pyridazin-3-one (230 mg, 0.65 mmol, 1.0 eq.) in DMF (5 mL) was added NIS (175 mg, 0.78 mmol, 1.2 eq.), the reaction mixture was stirred at 85° C. under N₂ atmosphere for 2 hrs. The resulting mixture was poured into ice water (20 mL), extracted with EtOAc (15 mL×3), the combined organic layers were washed with brine (20 mL), dried over anhydrous Na₂SO₄ and concentrated under reduced pressure. The resulting residue was purified by column chromatography on silica gel to give 4-chloro-5-(cyclopropylmethyl)-7-iodo-2-(2-methyl-2H-indazol-5-yl)-2,5-dihydro-3H-pyrrolo[3,2-c]pyridazin-3-one (270 mg, 88%) as a brown solid. LC-MS (ESI): m/z 480 [M+H]⁺.

### Step G: 4-chloro-5-(cyclopropylmethyl)-2-(2-methyl-2H-indazol-5-yl)-3-oxo-3,5-dihydro-2H-pyrrolo[3,2-c]pyridazine-7-carbonitrile

To a solution of 4-chloro-5-(cyclopropylmethyl)-7-iodo-2-(2-methyl-2H-indazol-5-yl)-2,5-dihydro-3H-pyrrolo[3,2-c]pyridazin-3-one (270 mg, 0.56 mmol, 1.0 eq.), Zn(CN)₂ (250 mg, 2.25 mmol, 4.0 eq.) and dppf (31 mg, 0.056 mmol, 0.1 eq.) in DMAc (5 mL) was added Pd(tBu₃P)₂ (28 mg, 0.056 mmol, 0.1 eq.), and the reaction mixture was stirred at 80° C. under N₂ atmosphere for 2 hrs. The resulting mixture was diluted with water (15 mL), extracted with EtOAc (10 mL×3), the combined organic layers were washed with brine (15 mL), dried over anhydrous Na₂SO₄ and concentrated under reduced pressure. The resulting residue was purified by column chromatography on silica gel to give 4-chloro-5-(cyclopropylmethyl)-2-(2-methyl-2H-indazol-5-yl)-3-oxo-3,5-dihydro-2H-pyrrolo[3,2-c]pyridazine-7-carbonitrile (173 mg, 82%) as a yellow solid. LC-MS (ESI): m/z 379 [M+H]⁺.

### Step H: 5-(cyclopropylmethyl)-2-(2-methyl-2H-indazol-5-yl)-3-oxo-4-(6-(trifluoromethyl)pyridin-3-yl)-3,5-dihydro-2H-pyrrolo[3,2-c]pyridazine-7-carbonitrile

To a solution of 4-chloro-5-(cyclopropylmethyl)-2-(2-methyl-2H-indazol-5-yl)-3-oxo-3,5-dihydro-2H-pyrrolo[3,2-c]pyridazine-7-carbonitrile (50 mg, 132 μmol, 1.0 eq.), (6-(trifluoromethyl)pyridin-3-yl)boronic acid (33 mg, 172 μmol, 1.3 eq.) and Pd(dppf)Cl₂ (10 mg, 13.2 μmol, 0.1 eq.) in dioxane (1 mL) was added a solution of K₂CO₃ (36 mg, 264 μmol, 2.0 eq.) in H₂O (0.3 mL), the reaction mixture was stirred at 100° C. under N₂ atmosphere for 4 hrs. The resulting mixture was filtered through a short pad of Celite®, the filtrate was concentrated under reduced pressure, the residue was purified by prep-HPLC to give 5-(cyclopropylmethyl)-2-(2-methyl-2H-indazol-5-yl)-3-oxo-4-(6-(trifluoromethyl)pyridin-3-yl)-3,5-dihydro-2H-pyrrolo[3,2-c]pyridazine-7-carbonitrile (Example 119). ¹H NMR (400 MHz, DMSO-d₆) δ (ppm): 8.94 (d, J=1.6 Hz, 1H), 8.77 (s, 1H), 8.48 (s, 1H), 8.31 (dd, J=8.1, 1.7 Hz, 1H), 8.09 (d, J=8.0 Hz, 1H), 7.97-7.93 (m, 1H), 7.69 (d, J=9.2 Hz, 1H), 7.40 (dd, J=9.2, 2.0 Hz, 1H), 4.21 (s, 3H), 3.36 (d, J=6.8 Hz, 2H), 0.72-0.67 (m, 1H), 0.40-0.35 (m, 2H), 0.17-0.13 (m, 2H). LC-MS (ESI): m/z 490 [M+H]⁺.

### Example 106 & 120—Synthesis of 5-(cyclopropylmethyl)-4-(6-cyclopropylpyridin-3-yl)-2-(2-methyl-2H-indazol-5-yl)-2,5-dihydro-3H-pyrrolo[3,2-c]pyridazin-3-one and 4-(6-(1-cyanocyclopropyl)pyridin-3-yl)-5-(cyclopropylmethyl)-2-(2-methyl-2H-indazol-5-yl)-3-oxo-3,5-dihydro-2H-pyrrolo[3,2-c]pyridazine-7-carbonitrile via General Procedure II (Method B)

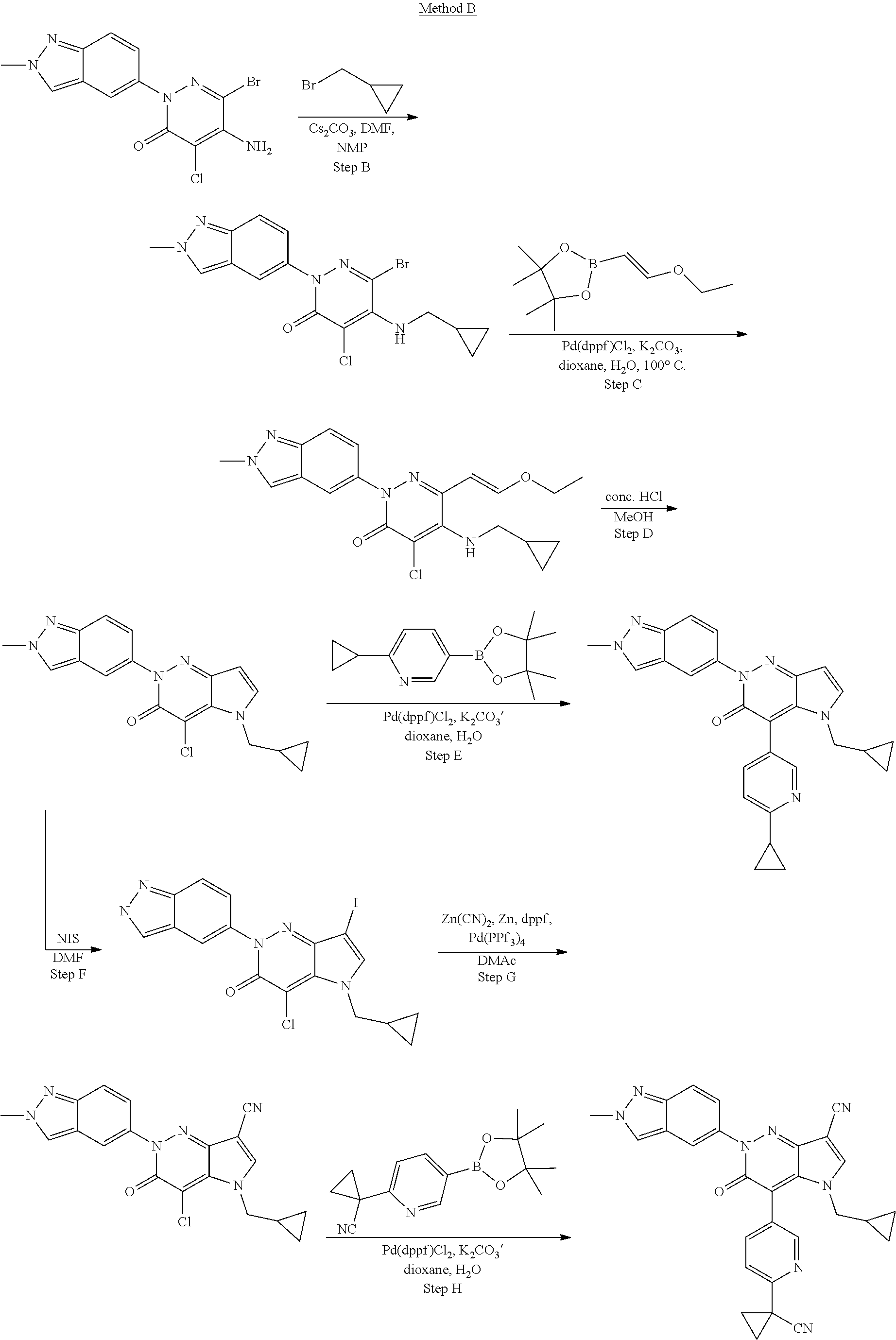

### Step B: 6-bromo-4-chloro-5-((cyclopropylmethyl)amino)-2-(2-methyl-2H-indazol-5-yl)pyridazin-3(2H)-one

To a solution of 5-amino-6-bromo-4-chloro-2-(2-methyl-2H-indazol-5-yl)-2,3-dihydropyridazin-3-one (5.0 g, 14.1 mmol, 1.0 eq.) in DMF (25 mL) and NMP (25 mL) was added (bromomethyl)cyclopropane (3.8 g, 28.2 mmol, 2.0 eq.) and Cs₂CO₃ (9.2 g, 28.2 mmol, 2.0 eq.), the reaction mixture was stirred at 15° C. under N₂ atmosphere overnight, The reaction mixture was poured into ice water (100 ml), a precipitation was formed, the mixture was filtered, the filter cake was washed with H₂O (50 mL) and then dried to give 6-bromo-4-chloro-5-((cyclopropylmethyl)amino)-2-(2-methyl-2H-indazol-5-yl)pyridazin-3(2H)-one (5.67 g, 98%) as a white solid. LC-MS (ESI): m/z 408, 410 [M+H]⁺.

### Step C: (E)-4-chloro-5-((cyclopropylmethyl)amino)-6-(2-ethoxyvinyl)-2-(2-methyl-2H-indazol-5-yl)pyridazin-3(2H)-one

To a solution of 6-bromo-4-chloro-5-((cyclopropylmethyl)amino)-2-(2-methyl-2H-indazol-5-yl)pyridazin-3(2H)-one (5.38 g, 13.1 mmol, 1.0 eq.) and (E)-2-(2-ethoxyvinyl)-4,4,5,5-tetramethyl-1,3,2-dioxaborolane (2.87 g, 14.5 mmol, 1.1 eq.) in dioxane (55 mL) was added a solution of K₂CO₃ (3.64 g, 26.3 mmol, 2.0 eq.) in H₂O (11 mL). The reaction mixture was degassed with N₂ three times before Pd(dppf)Cl₂ (0.48 g, 0.658 mmol, 0.05 eq.) was added. The reaction mixture was stirred at 100° C. under N₂ atmosphere for 3 hrs. The reaction mixture was concentrated under reduced pressure, and the residue was purified by flash column chromatography on silica gel to give (E)-4-chloro-5-((cyclopropylmethyl)amino)-6-(2-ethoxyvinyl)-2-(2-methyl-2H-indazol-5-yl)pyridazin-3(2H)-one (5.13 g, 98%) as a white solid. LC-MS (ESI): m/z 400 [M+H]⁺.

### Step D: 4-chloro-5-(cyclopropylmethyl)-2-(2-methyl-2H-indazol-5-yl)-2,5-dihydro-3H-pyrrolo[3,2-c]pyridazin-3-one

A solution of (E)-4-chloro-5-((cyclopropylmethyl)amino)-6-(2-ethoxyvinyl)-2-(2-methyl-2H-indazol-5-yl)pyridazin-3(2H)-one(5.13 g, 12.8 mmol, 1.0 eq.) in MeOH (50 mL) and conc. HCl (0.5 mL) was stirred at 80° C. for 4 hrs. The reaction mixture was concentrated under reduced pressure, the residue was diluted with H₂O (30 mL), then adjusted pH=7 with solid NaHCO₃, which caused a precipitate to form. The solid was collected and purified by flash column chromatography on silica gel to give 4-chloro-5-(cyclopropylmethyl)-2-(2-methyl-2H-indazol-5-yl)-2,5-dihydro-3H-pyrrolo[3,2-c]pyridazin-3-one (3.55 g, 78%) as a yellow solid. LC-MS (ESI):m/z 354 [M+H]⁺.

### Step E: 5-(cyclopropylmethyl)-4-(6-cyclopropylpyridin-3-yl)-2-(2-methyl-2H-indazol-5-yl)-2,5-dihydro-3H-pyrrolo[3,2-c]pyridazin-3-one

To a solution of 4-chloro-5-(cyclopropylmethyl)-2-(2-methyl-2H-indazol-5-yl)-2,5-dihydro-3H-pyrrolo[3,2-c]pyridazin-3-one (806 mg, 2.3 mmol, 1.0 eq.), (6-cyclopropylpyridin-3-yl)boronic acid (446 mg, 2.7 mmol, 1.2 eq.) and Pd(dppf)Cl₂ (83 mg, 0.1 mmol, 0.05 eq.) in dioxane (8 mL) was added a solution of K₂CO₃ (630 mg, 4.6 mmol, 2.0 eq.) in H₂O (2 mL). The reaction mixture was degassed with N₂ three times and stirred at 100° C. under N₂ atmosphere for 5 hrs. The reaction mixture was concentrated under reduced pressure, and the residue was purified by flash column chromatography on silica gel to give 5-(cyclopropylmethyl)-4-(6-cyclopropylpyridin-3-yl)-2-(2-methyl-2H-indazol-5-yl)-2,5-dihydro-3H-pyrrolo[3,2-c]pyridazin-3-one (Example 106, alternative route). ¹H NMR (400 MHz, DMSO-d₆) δ (ppm): 8.39 (d, J=1.9 Hz, 1H), 8.34 (s, 1H), 7.81 (d, J=1.4 Hz, 1H), 7.69 (dd, J=8.0, 2.2 Hz, 1H), 7.62 (d, J=3.8 Hz, 1H), 7.55 (d, J=9.2 Hz, 1H), 7.33 (d, J=8.0 Hz, 1H), 7.29 (dd, J=9.2, 2.0 Hz, 1H), 6.36 (d, J=3.8 Hz, 1H), 4.12 (s, 3H), 3.23 (d, J=6.7 Hz, 2H), 2.11-2.07 (m, 1H), 1.02-0.86 (m, 4H), 0.61-0.52 (m, 1H), 0.25-0.19 (m, 2H), −0.01-0.05 (m, 2H). LC-MS (ESI): m/z 437 [M+H]⁺.

### Step F: 4-chloro-5-(cyclopropylmethyl)-7-iodo-2-(2-methyl-2H-indazol-5-yl)-2,5-dihydro-3H-pyrrolo[3,2-c]pyridazin-3-one

To a solution of 4-chloro-5-(cyclopropylmethyl)-2-(2-methyl-2H-indazol-5-yl)-2H,3H,5H-pyrrolo[3,2-c]pyridazin-3-one (230 mg, 0.65 mmol, 1.0 eq.) in DMF (5 mL) was added NIS (175 mg, 0.78 mmol, 1.2 eq.), and the reaction mixture was stirred at 85° C. under N₂ atmosphere for 2 hrs. The resulting mixture was poured into ice water (20 mL), extracted with EtOAc (15 mL×3), the combined organic layers were washed with brine (20 mL), dried over anhydrous Na₂SO₄ and concentrated under reduced pressure. The resulting residue was purified by column chromatography on silica gel to give 4-chloro-5-(cyclopropylmethyl)-7-iodo-2-(2-methyl-2H-indazol-5-yl)-2,5-dihydro-3H-pyrrolo[3,2-c]pyridazin-3-one (270 mg, 88%) as a brown solid. LC-MS (ESI): m/z 480 [M+H]⁺.

### Step G: 4-chloro-5-(cyclopropylmethyl)-2-(2-methyl-2H-indazol-5-yl)-3-oxo-3,5-dihydro-2H-pyrrolo[3,2-c]pyridazine-7-carbonitrile

To a solution of 4-chloro-5-(cyclopropylmethyl)-7-iodo-2-(2-methyl-2H-indazol-5-yl)-2,5-dihydro-3H-pyrrolo[3,2-c]pyridazin-3-one (270 mg, 0.56 mmol, 1.0 eq.), Zn(CN)₂ (250 mg, 2.25 mmol, 4.0 eq.) and dppf (31 mg, 0.056 mmol, 0.1 eq.) in DMAc (5 mL) was added Pd(tBu₃P)₂ (28 mg, 0.056 mmol, 0.1 eq.), and the reaction mixture was stirred at 80° C. under N₂ atmosphere for 2 hrs. The resulting mixture was diluted with water (15 mL), extracted with EtOAc (10 mL×3), the combined organic layers were washed with brine (15 mL), dried over anhydrous Na₂SO₄ and concentrated under reduced pressure. The resulting residue was purified by column chromatography on silica gel to give 4-chloro-5-(cyclopropylmethyl)-2-(2-methyl-2H-indazol-5-yl)-3-oxo-3,5-dihydro-2H-pyrrolo[3,2-c]pyridazine-7-carbonitrile (173 mg, 82%) as a yellow solid. LC-MS (ESI): m/z 379 [M+H]⁺.

### Step H: 4-(6-(1-cyanocyclopropyl)pyridin-3-yl)-5-(cyclopropylmethyl)-2-(2-methyl-2H-indazol-5-yl)-3-oxo-3,5-dihydro-2H-pyrrolo[3,2-c]pyridazine-7-carbonitrile

To a solution of 4-chloro-5-(cyclopropylmethyl)-2-(2-methyl-2H-indazol-5-yl)-3-oxo-3,5-dihydro-2H-pyrrolo[3,2-c]pyridazine-7-carbonitrile (50 mg, 132 μmol, 1.0 eq.), 1-(5-(4,4,5,5-tetramethyl-1,3,2-dioxaborolan-2-yl)pyridin-2-yl)cyclopropane-1-carbonitrile (46 mg, 172 μmol, 1.3 eq.) and Pd(dppf)Cl₂ (10 mg, 13.2 μmol, 0.1 eq.) in dioxane (1 mL) was added a solution of K₂CO₃ (36 mg, 264 μmol, 2.0 eq.) in H₂O (0.3 mL), and the reaction mixture was stirred at 100° C. under N₂ atmosphere for 4 hrs. The resulting mixture was filtered through a short pad of Celite®, the filtrate was concentrated under reduced pressure, and the residue was purified by prep-HPLC to give 4-(6-(1-cyanocyclopropyl)pyridin-3-yl)-5-(cyclopropylmethyl)-2-(2-methyl-2H-indazol-5-yl)-3-oxo-3,5-dihydro-2H-pyrrolo[3,2-c]pyridazine-7-carbonitrile Example 120). ¹H NMR (400 MHz, DMSO-d₆) δ (ppm): 8.73 (s, 1H), 8.65 (s, 1H), 8.47 (s, 1H), 8.02 (d, J=7.6 Hz, 1H), 7.94 (s, 1H), 7.67 (d, J=9.2 Hz, 2H), 7.38 (d, J=9.2 Hz, 1H), 4.21 (s, 3H), 3.38-3.33 (m, 2H), 1.93-1.85 (m, 2H), 1.78-1.69 (m, 2H), 0.75-0.65 (m, 1H), 0.41-0.34 (m, 2H), 0.19-0.11 (m, 2H). LC-MS (ESI): m/z 487 [M+H]⁺.

The procedure set forth above for General Procedure II (Method A) was used to synthesize the following compounds by using appropriate starting materials:

| Example | Structure | Characterization |
|---|---|---|
| 121 | 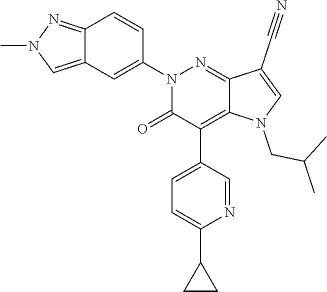 [SMILES: CC(C)Cn1cc(C#N)c2nn(-c3ccc4nn(C)cc4c3)c(=O)c(-c3ccc(C4CC4)nc3)c21] | LC-MS: m/z 464 (M + H)⁺. ¹H NMR (400 MHz, DMSO- d₆) δ: 8.63 (s, 1H), 8.49 (d, J = 2.1 Hz, 1H), 8.46 (s, 1H), 7.94 (d, J = 1.4 Hz, 1H), 7.79 (dd, J = 8.1, 2.1 Hz, 1H), 7.67 (d, J = 9.1 Hz, 1H), 7.44 (d, J = 8.1 Hz, 1H), 7.38 (dd, J = 9.1, 2.0 Hz, 1H), 4.21 (s, 3H), 3.40 (d, J = 7.7 Hz, 2H), 2.21-2.15 (m, 1H), 1.32-1.23 (m, 1H), 1.05- 0.91 (m, 4H), 0.46 (d, J = 6.4 Hz, 6H). |
| | 4-(6-cyclopropylpyridin-3-yl)-5-isobutyl-2-(2-methyl- | |
| | 2H-indazol-5-yl)-3-oxo-3,5-dihydro-2H-pyrrolo[3,2- | |
| | c]pyridazine-7-carbonitrile | |
| 122 | 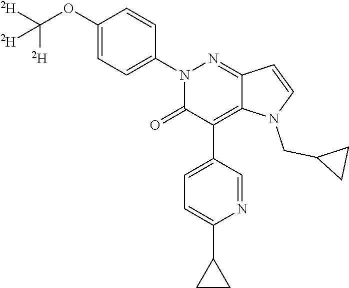 [SMILES: [2H]C([2H])([2H])Oc1ccc(-n2nc3ccn(CC4CC4)c3c(-c3ccc(C4CC4)nc3)c2=O)cc1] | LC-MS: m/z 416 (M + H)⁺. ¹H NMR (400 MHz, DMSO- d₆) δ: 8.45 (d, J = 1.8 Hz, 1H), 7.75 (dd, J = 8.0, 2.2 Hz, 1H), 7.69 (d, J = 3.8 Hz, 1H), 7.48 (d, J = 9.2 Hz, 2H), 7.41 (d, J = 7.8 Hz, 1H), 7.01 (d, J = 9.2 Hz, 2H), 6.43 (d, J = 3.8 Hz, 1H), 3.31 (d, J = 7.2 Hz, 2H), 2.23-2.12 (m, 1H), 1.05-0.90 (m, 4H), 0.68-0.57 (m, 1H), 0.32-0.24 (m, 2H), 0.09-0.04 (m, 2H). |
| | 5-(cyclopropylmethyl)-4-(6-cyclopropylpyridin-3-yl)-2- | |
| | (4-(methoxy-d3)phenyl)-2,5-dihydro-3H-pyrrolo[3,2- | |
| | c]pyridazin-3-one | |
| 123 | 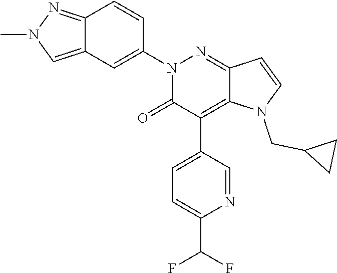 | LC-MS: m/z 416 (M + H)⁺. ¹H NMR (400 MHz, DMSO- d₆) δ: 8.82 (s, 1H), 8.44 (s, 1H), 8.28-8.12 (m, 1H), 7.91 (d, J = 1.4 Hz, 1H), 7.83 (d, J = 7.9 Hz, 1H), 7.75 (d, J = 3.8 Hz, 1H), 7.65 (d, J = 9.1 Hz, 1H), 7.38 (dd, J = 9.2, 2.0 Hz, 1H), 7.07 (t, J_{HF} = 54.9 Hz, 1H), 6.50 (d, J = 3.8 Hz, 1H), 4.20 (s, 3H), 3.32 (d, J = 7.2 Hz, 2H), 0.68-0.57 (m, 1H), 0.34-0.25 (m, 2H), 0.10-0.05 (m, 2H). |
| | 5-(cyclopropylmethyl)-4-(6-(difluoromethyl)pyridin-3-yl)- | |
| | 2-(2-methyl-2H-indazol-5-yl)-2,5-dihydro-3H-pyrrolo[3,2- | |
| | c]pyridazin-3-one | |
| 124 | 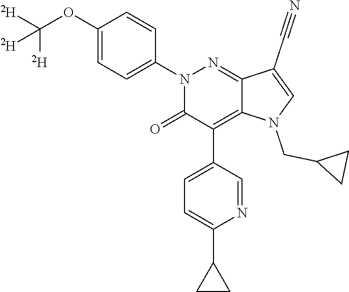 [SMILES: [2H]C([2H])([2H])Oc1ccc(-n2nc3c(C#N)cn(CC4CC4)c3c(-c3ccc(C4CC4)nc3)c2=O)cc1] | LC-MS: m/z 441 (M + H)⁺. ¹H NMR (400 MHz, CDCl₃) δ: 8.42 (s, 1H), 7.93 (s, 1H), 7.71 (dd, J = 8.0, 1.8 Hz, 1H), 7.60 (d, J = 9.0 Hz, 2H), 7.24 (d, J = 8.2 Hz, 1H), 6.97 (d, J = 9.0 Hz, 2H), 3.34 (d, J = 6.9 Hz, 2H), 2.15-2.04 (m, 1H), 1.11-1.02 (m, 4H), 0.89-0.78 (m, 1H), 0.60-0.52 (m, 2H), 0.16-0.13 (m, 2H). |
| | 5-(cyclopropylmethyl)-4-(6-cyclopropylpyridin-3-yl)-2-(4- | |
| | (methoxy-d3)phenyl)-3-oxo-3,5-dihydro-2H-pyrrolo[3,2- | |
| | c]pyridazine-7-carbonitrile | |
| 125 | 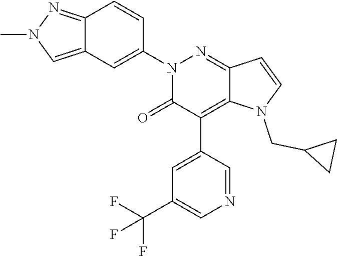 [SMILES: Cn1cc2cc(-n3nc4ccn(CC5CC5)c4c(-c4cncc(C(F)(F)F)c4)c3=O)ccc2n1] | LC-MS: m/z 465 (M + H)⁺. ¹H NMR (400 MHz, DMSO- d₆) δ: 9.06 (s, 1H), 9.03 (s, 1H), 8.49 (s, 1H), 8.44 (s, 1H), 7.91 (s, 1H), 7.75 (d, J = 3.8 Hz, 1H), 7.65 (d, J = 9.2 Hz, 1H), 7.39 (dd, J = 9.2, 1.9 Hz, 1H), 6.51 (d, J = 3.8 Hz, 1H), 4.20 (s, 3H), 3.28 (s, 2H), 0.70-0.65 (m, 1H), 0.35- 0.28 (m, 2H), 0.12-0.07 (m, 2H). |
| | 5-(cyclopropylmethyl)-2-(2-methyl-2H-indazol-5-yl)-4- | |
| | (5-(trifluoromethyl)pyridin-3-yl)-2,5-dihydro-3H- | |
| | pyrrolo[3,2-c]pyridazin-3-one | |
| 126 | 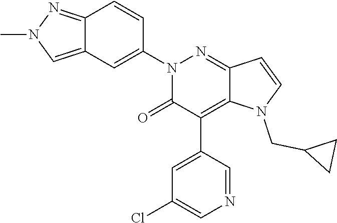 [SMILES: Cn1cc2cc(-n3nc4ccn(CC5CC5)c4c(-c4cncc(Cl)c4)c3=O)ccc2n1] | LC-MS: m/z 431 (M + H)⁺. ¹H NMR (400 MHz, DMSO- d₆) δ: 8.60 (s, 1H), 8.55 (s, 1H), 8.31 (s, 1H), 8.07 (d, J = 1.8 Hz, 1H), 7.77 (d, J = 1.3 Hz, 1H), 7.61 (d, J = 3.8 Hz, 1H), 7.52 (d, J = 9.1 Hz, 1H), 7.25 (dd, J = 9.1, 1.9 Hz, 1H), 6.36 (d, J = 3.7 Hz, 1H), 4.07 (s, 3H), 3.17 (d, J = 7.2 Hz, 2H), 0.65-0.50 (m, 1H), 0.22- 0.19 (m, 2H), −0.01-0.0 (m, 2H). |
| | 4-(5-chloropyridin-3-yl)-5-(cyclopropylmethyl)-2-(2- | |
| | methyl-2H-indazol-5-yl)-2,5-dihydro-3H-pyrrolo[3,2- | |
| | c]pyridazin-3-one | |
| 127 | 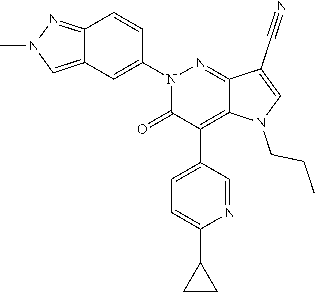 [SMILES: CCCn1cc(C#N)c2nn(-c3ccc4nn(C)cc4c3)c(=O)c(-c3ccc(C4CC4)nc3)c21] | LC-MS: m/z 450 (M + H)⁺. ¹H NMR (400 MHz, DMSO- d₆) δ: 8.64 (s, 1H), 8.49 (s, 1H), 8.46 (s, 1H), 7.93 (s, 1H), 7.83-7.75 (m, 1H), 7.67 (d, J = 9.2 Hz, 1H), 7.44 (d, J = 8.0 Hz, 1H), 7.38 (d, J = 9.2 Hz, 1H), 4.21 (s, 3H), 3.48 (t, J = 7.2 Hz, 2H), 2.22-2.12 (m, 1H), 1.27-1.25 (m, 2H), 1.07- 0.92 (m, 4H), 0.45 (t, J = 7.2 Hz, 3H). |
| | 4-(6-cyclopropylpyridin-3-yl)-2-(2-methyl-2H- | |
| | indazol-5-yl)-3-oxo-5-propyl-3,5-dihydro-2H- | |
| | pyrrolo[3,2-c]pyridazine-7-carbonitrile | |
| 128 | 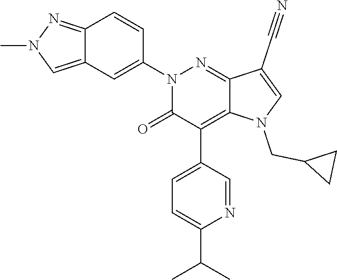 [SMILES: CC(C)c1ccc(-c2c(=O)n(-c3ccc4nn(C)cc4c3)nc3c(C#N)cn(CC4CC4)c23)cn1] | LC-MS: m/z 464 (M + H)⁺. ¹H NMR (400 MHz, DMSO- d₆) δ: 8.71 (s, 1H), 8.60 (d, J = 1.8 Hz, 1H), 8.47 (s, 1H), 7.95 (d, J = 1.4 Hz, 1H), 7.88 (dd, J = 8.0, 2.1 Hz, 1H), 7.69 (s, 1H), 7.42 (d, J = 8.1 Hz, 1H), 7.39 (dd, J = 9.2, 2.0 Hz, 1H), 4.21 (s, 3H), 3.37 (d, J = 7.1 Hz, 2H), 3.15-3.06 (m, 1H), 1.28 (d, J = 6.8 Hz, 6H), 0.70- 0.65 (m, 1H), 0.36-0.30 (m, 2H), 0.11-0.06 (m, 2H). |
| | 5-(cyclopropylmethyl)-4-(6-isopropylpyridin-3-yl)-2-(2- | |
| | methyl-2H-indazol-5-yl)-3-oxo-3,5-dihydro-2H- | |
| | pyrrolo[3,2-c]pyridazine-7-carbonitrile | |
| 129 | 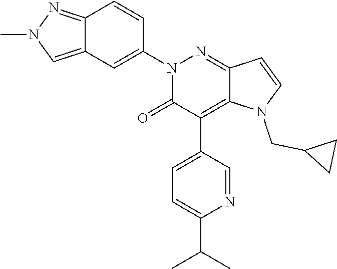 [SMILES: CC(C)c1ccc(-c2c(=O)n(-c3ccc4nn(C)cc4c3)nc3ccn(CC4CC4)c23)cn1] | LC-MS: m/z 439 (M + H)⁺. ¹H NMR (400 MHz, DMSO- d₆) δ: 8.57 (d, J = 1.7 Hz, 1H), 8.43 (s, 1H), 7.89 (d, J = 1.3 Hz, 1H), 7.84 (dd, J = 8.0, 2.2 Hz, 1H), 7.70 (d, J = 3.8 Hz, 1H), 7.64 (d, J = 9.2 Hz, 1H), 7.43-7.33 (m, 2H), 6.45 (d, J = 3.8 Hz, 1H), 4.20 (s, 3H), 3.30 (d, J = 7.2 Hz, 2H), 3.14-3.07 (m, 1H), 1.28 (d, J = 6.9 Hz, 6H), 0.64-0.58 (m, 1H), 0.33- 0.20 (m, 2H), 0.05-0.01 (m, 2H). |
| | 5-(cyclopropylmethyl)-4-(6-isopropylpyridin-3-yl)-2-(2- | |
| | methyl-2H-indazol-5-yl)-2,5-dihydro-3H-pyrrolo[3,2- | |
| | c]pyridazin-3-one | |
| 130 | 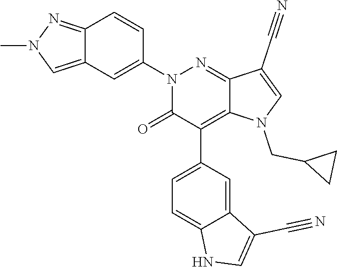 [SMILES: Cn1cc2cc(-n3nc4c(C#N)cn(CC5CC5)c4c(-c4ccc5[nH]cc(C#N)c5c4)c3=O)ccc2n1] | LC-MS: m/z 485 (M + H)⁺. ¹H NMR (400 MHz, DMSO- d₆) δ: 12.36 (s, 1H), 8.69 (s, 1H), 8.46 (s, 1H), 8.35 (s, 1H), 7.96 (s, 1H), 7.79 (s, 1H), 7.67 (dd, J = 8.8, 4.8 Hz, 2H), 7.44-7.35 (m, 2H), 4.21 (s, 3H), 3.30 (d, J = 7.2 Hz, 2H), 0.78-0.66 (m, 1H), 0.34- 0.26 (m, 2H), 0.08-0.03 (m, 2H). |
| | 4-(3-cyano-1H-indol-5-yl)-5-(cyclopropylmethyl)-2-(2- | |
| | methyl-2H-indazol-5-yl)-3-oxo-3,5-dihydro-2H- | |
| | pyrrolo[3,2-c]pyridazine-7-carbonitrile | |
| 131 | 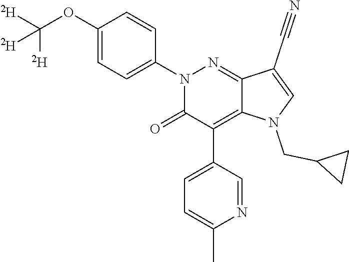 [SMILES: [2H]C([2H])([2H])Oc1ccc(-n2nc3c(C#N)cn(CC4CC4)c3c(-c3ccc(C)nc3)c2=O)cc1] | LC-MS: m/z 415 (M + H)⁺. ¹H NMR (400 MHz, DMSO- d₆) δ: 8.70 (s, 1H), 8.53 (d, J = 1.8 Hz, 1H), 7.83 (dd, J = 7.9, 2.2 Hz, 1H), 7.52 (d, J = 8.9 Hz, 2H), 7.40 (d, J = 7.9 Hz, 1H), 7.05 (d, J = 8.9 Hz, 2H), 3.38 (d, J = 7.2 Hz, 2H), 2.55 (s, 3H), 0.76-0.66 (m, 1H), 0.37-0.32 (m, 2H), 0.15-0.11 (m, 2H). |
| | 5-(cyclopropylmethyl)-2-(4-(methoxy-d3)phenyl)-4-(6- | |
| | methylpyridin-3-yl)-3-oxo-3,5-dihydro-2H-pyrrolo[3,2- | |
| | c]pyridazine-7-carbonitrile | |
| 132 | 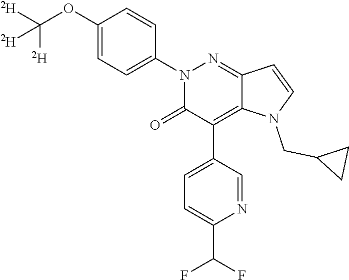 [SMILES: [2H]C([2H])([2H])Oc1ccc(-n2nc3ccn(CC4CC4)c3c(-c3ccc(C(F)F)nc3)c2=O)cc1] | LC-MS: m/z 426 (M + H)⁺. ¹H NMR (400 MHz, DMSO- d₆) δ: 8.80 (s, 1H), 8.16 (d, J = 7.1 Hz, 1H), 7.82 (d, J = 8.0 Hz, 1H), 7.72 (d, J = 3.6 Hz, 1H), 7.50 (d, J = 8.8 Hz, 2H), 7.05 (t, J_{HF} = 55.0 Hz, 1H), 7.02 (d, J = 8.8 Hz, 2H), 6.47 (d, J = 3.7 Hz, 1H), 3.29 (d, J = 7.2 Hz, 2H), 0.66-0.58 (m, 1H), 0.31-0.25 (m, 2H), 0.08- 0.02 (m, 2H). |
| | 5-(cyclopropylmethyl)-4-(6-(difluoromethyl)pyridin-3-yl)- | |
| | 2-(4-(methoxy-d3)phenyl)-2,5-dihydro-3H-pyrrolo[3,2- | |
| | c]pyridazin-3-one | |
| 133 | 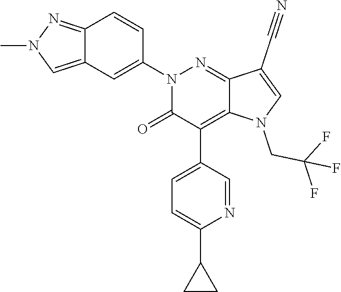 [SMILES: Cn1cc2cc(-n3nc4c(C#N)cn(CC(F)(F)F)c4c(-c4ccc(C5CC5)nc4)c3=O)ccc2n1] | LC-MS: m/z 490 (M + H)⁺. ¹H NMR (400 MHz, DMSO- d₆) δ: 8.69 (s, 1H), 8.47 (s, 1H), 8.46 (d, J = 2.0 Hz, 1H), 7.96 (d, J = 1.6 Hz, 1H), 7.75 (dd, J = 8.0, 2.0 Hz, 1H), 7.68 (d, J = 9.2 Hz, 1H), 7.46 (d, J = 8.0 Hz, 1H), 7.39 (dd, J = 9.2, 2.0 Hz, 1H), 4.58-4.44 (m, 2H), 4.21 (s, 3H), 2.20- 2.17 (m, 1H), 1.08-0.94 (m, 4H). |
| | 4-(6-cyclopropylpyridin-3-yl)-2-(2-methyl-2H-indazol-5- | |
| | yl)-3-oxo-5-(2,2,2-trifluoroethyl)-3,5-dihydro-2H- | |
| | pyrrolo[3,2-c]pyridazine-7-carbonitrile | |
| 134 | 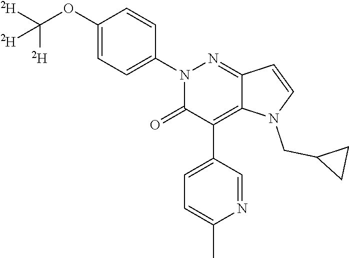 [SMILES: [2H]C([2H])([2H])Oc1ccc(-n2nc3ccn(CC4CC4)c3c(-c3ccc(C)nc3)c2=O)cc1] | LC-MS: m/z 390 (M + H)⁺. ¹H NMR (400 MHz, DMSO- d₆) δ: 8.51 (d, J = 1.9 Hz, 1H), 7.80 (dd, J = 7.9, 2.2 Hz, 1H), 7.69 (d, J = 3.8 Hz, 1H), 7.49 (d, J = 8.9 Hz, 2H), 7.37 (d, J = 8.0 Hz, 1H), 7.01 (d, J = 8.9 Hz, 2H), 6.43 (d, J = 3.7 Hz, 1H), 3.33 (d, J = 7.2 Hz, 2H), 2.54 (s, 3H), 0.75-0.60 (m, 1H), 0.35-0.22 (m, 2H), 0.10- 0.04 (m, 2H). |
| | 5-(cyclopropylmethyl)-2-(4-(methoxy-d3)phenyl)-4-(6- | |
| | methylpyridin-3-yl)-2,5-dihydro-3H-pyrrolo[3,2- | |
| | c]pyridazin-3-one | |
| 135 | 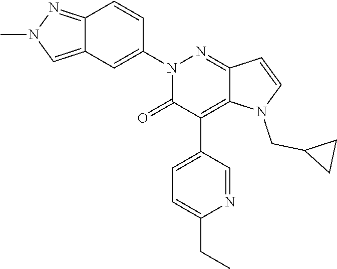 [SMILES: CCc1ccc(-c2c(=O)n(-c3ccc4nn(C)cc4c3)nc3ccn(CC4CC4)c23)cn1] | LC-MS: m/z 425 (M + H)⁺. ¹H NMR (400 MHz, DMSO- d₆) δ: 8.58 (d, J = 1.8 Hz, 1H), 8.43 (s, 1H), 7.94-7.84 (m, 2H), 7.72 (d, J = 3.8 Hz, 1H), 7.64 (d, J = 9.2 Hz, 1H), 7.43 (d, J = 8.0 Hz, 1H), 7.37 (dd, J = 9.1, 2.0 Hz, 1H), 6.46 (d, J = 3.8 Hz, 1H), 4.20 (s, 3H), 3.33 (d, J = 7.1 Hz, 2H), 2.84 (q, J = 7.5 Hz, 2H), 1.27 (t, J = 7.6 Hz, 3H), 0.70-0.59 (m, 1H), 0.34-0.24 (m, 2H), 0.11- 0.01 (m, 2H). |
| | 5-(cyclopropylmethyl)-4-(6-ethylpyridin-3-yl)-2-(2-methyl- | |
| | 2H-indazol-5-yl)-2,5-dihydro-3H-pyrrolo[3,2-c]pyridazin- | |
| | 3-one | |
| 136 | 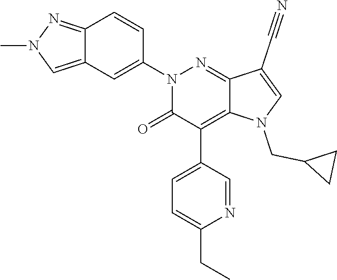 [SMILES: CCc1ccc(-c2c(=O)n(-c3ccc4nn(C)cc4c3)nc3c(C#N)cn(CC4CC4)c23)cn1] | LC-MS: m/z 450 (M + H)⁺. ¹H NMR (400 MHz, DMSO- d₆) δ: 8.72 (s, 1H), 8.59 (d, J = 1.9 Hz, 1H), 8.47 (s, 1H), 7.95 (d, J = 1.5 Hz, 1H), 7.87 (dd, J = 8.0, 2.2 Hz, 1H), 7.68 (d, J = 9.2 Hz, 1H), 7.43-7.37 (m, 2H), 4.21 (s, 3H), 3.38 (d, J = 7.2 Hz, 2H), 2.83 (q, J = 7.5 Hz, 2H), 1.27 (t, J = 7.5 Hz, 3H), 0.76-0.66 (m, 1H), 0.41- 0.30 (m, 2H), 0.19-0.07 (m, 2H). |
| | 5-(cyclopropylmethyl)-4-(6-ethylpyridin-3-yl)-2-(2- | |
| | methyl-2H-indazol-5-yl)-3-oxo-3,5-dihydro-2H- | |
| | pyrrolo[3,2-c]pyridazine-7-carbonitrile | |
| 137 | 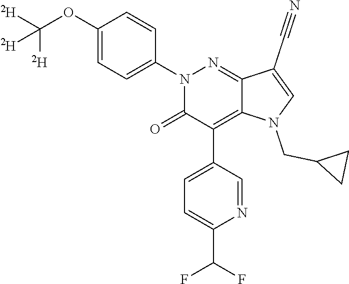 [SMILES: [2H]C([2H])([2H])Oc1ccc(-n2nc3c(C#N)cn(CC4CC4)c3c(-c3ccc(C(F)F)nc3)c2=O)cc1] | LC-MS: m/z 425 (M + H)⁺. ¹H NMR (400 MHz, DMSO- d₆) δ: 8.82 (s, 1H), 8.74 (s, 1H), 8.18 (dd, J = 8.0, 1.9 Hz, 1H), 7.86 (d, J = 8.0 Hz, 1H), 7.58-7.50 (m, 2H), 7.06 (t, J_{HF} = 54.8 Hz, 1H), 7.11-7.03 (m, 2H), 3.35 (d, J = 6.9 Hz, 2H), 0.70-0.65 (m, 1H), 0.38- 0.33 (m, 2H), 0.16-0.10 (m, 2H). |
| | 5-(cyclopropylmethyl)-4-(6-(difluoromethyl)pyridin-3-yl)- | |
| | 2-(4-(methoxy-d3)phenyl)-3-oxo-3,5-dihydro-2H- | |
| | pyrrolo[3,2-c]pyridazine-7-carbonitrile | |
| 138 | 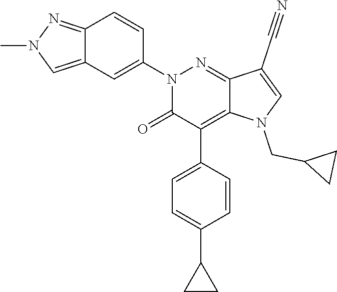 [SMILES: Cn1cc2cc(-n3nc4c(C#N)cn(CC5CC5)c4c(-c4ccc(C5CC5)cc4)c3=O)ccc2n1] | LC-MS: m/z 461 (M + H)⁺. ¹H NMR (400 MHz, DMSO- d₆) δ: 8.68 (s, 1H), 8.46 (s, 1H), 7.95-7.90 (m, 1H), 7.67 (d, J = 9.1 Hz, 1H), 7.39-7.34 (m, 3H), 7.19 (d, J = 8.2 Hz, 2H), 4.21 (s, 3H), 3.37 (d, J = 7.2 Hz, 2H), 2.04-1.96 (m, 1H), 1.04-0.98 (m, 2H), 0.80- 0.65 (m, 3H), 0.36-0.27 (m, 2H), 0.12-0.08 (m, 2H) |
| | 5-(cyclopropylmethyl)-4-(4-cyclopropylphenyl)-2-(2- | |
| | methyl-2H-indazol-5-yl)-3-oxo-3,5-dihydro-2H- | |
| | pyrrolo[3,2-c]pyridazine-7-carbonitrile | |
| 139 | 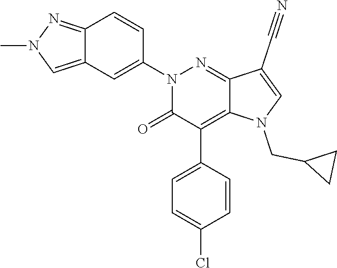 [SMILES: Cn1cc2cc(-n3nc4c(C#N)cn(CC5CC5)c4c(-c4ccc(Cl)cc4)c3=O)ccc2n1] | LC-MS: m/z 455 (M + H)⁺. ¹H NMR (400 MHz, DMSO- d₆) δ: 8.71 (s, 1H), 8.46 (s, 1H), 7.94 (d, J = 1.4 Hz, 1H), 7.68 (d, J = 9.2 Hz, 1H), 7.56 (d, J = 8.7 Hz, 4H), 7.38 (dd, J = 9.2, 2.0 Hz, 1H), 4.21 (s, 3H), 3.37 (d, J = 7.2 Hz, 2H), 0.76-0.68 (m, 1H), 0.39-0.34 (m, 2H), 0.17-0.10 (m, 2H). |
| | 4-(4-chlorophenyl)-5-(cyclopropylmethyl)-2-(2-methyl- | |
| | 2H-indazol-5-yl)-3-oxo-3,5-dihydro-2H-pyrrolo[3,2- | |
| | c]pyridazine-7-carbonitrile | |
| 140 | 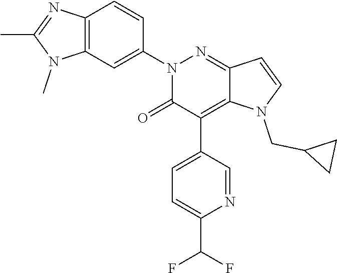 [SMILES: Cc1nc2ccc(-n3nc4ccn(CC5CC5)c4c(-c4ccc(C(F)F)nc4)c3=O)cc2n1C] | LC-MS: m/z 461 (M + H)⁺. ¹H NMR (400 MHz, DMSO- d₆) δ: 8.82 (d, J = 1.5 Hz, 1H), 8.18 (dd, J = 8.0, 1.9 Hz, 1H), 7.83 (d, J = 8.0 Hz, 1H), 7.74 (d, J = 3.8 Hz, 1H), 7.70 (d, J = 1.8 Hz, 1H), 7.57 (d, J = 8.5 Hz, 1H), 7.30 (dd, J = 8.5, 2.0 Hz, 1H), 7.06 (t, J_{HF} = 54.9 Hz, 1H), 6.50 (d, J = 3.8 Hz, 1H), 3.74 (s, 3H), 3.36 (d, J = 7.2 Hz, 2H), 2.55 (s, 3H), 0.70-0.59 (m, 1H), 0.33-0.28 (m, 2H), 0.10-0.06 (m, 2H). |
| | 5-(cyclopropylmethyl)-4-(6-(difluoromethyl)pyridin-3- | |
| | yl)-2-(1,2-dimethyl-1H-benzo[d]imidazol-6-yl)-2,5- | |
| | dihydro-3H-pyrrolo[3,2-c]pyridazin-3-one | |
| 141 | 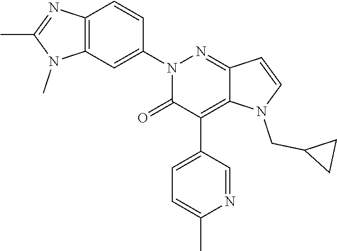 [SMILES: Cc1ccc(-c2c(=O)n(-c3ccc4nc(C)n(C)c4c3)nc3ccn(CC4CC4)c23)cn1] | LC-MS: m/z 425 (M + H)⁺. ¹H NMR (400 MHz, DMSO- d₆) δ: 8.52 (d, J = 1.9 Hz, 1H), 7.82 (dd, J = 7.9, 2.3 Hz, 1H), 7.70 (dd, J = 6.1, 2.8 Hz, 2H), 7.56 (d, J = 8.5 Hz, 1H), 7.38 (d, J = 8.0 Hz, 1H), 7.28 (dd, J = 8.5, 2.0 Hz, 1H), 6.45 (d, J = 3.8 Hz, 1H), 3.74 (s, 3H), 3.35 (d, J = 7.2 Hz, 2H), 2.55 (s, 3H), 2.54 (s, 3H), 0.68- 0.57 (m, 1H), 0.34-0.26 (m, 2H), 0.08-0.04 (m, 2H). |
| | 5-(cyclopropylmethyl)-2-(1,2-dimethyl-1H- | |
| | benzo[d]imidazol-6-yl)-4-(6-methylpyridin-3-yl)-2,5- | |
| | dihydro-3H-pyrrolo[3,2-c]pyridazin-3-one | |
| 142 | 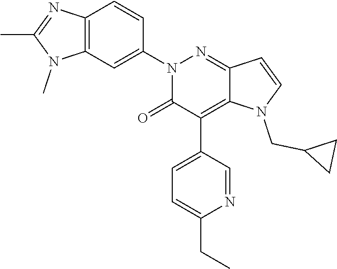 [SMILES: CCc1ccc(-c2c(=O)n(-c3ccc4nc(C)n(C)c4c3)nc3ccn(CC4CC4)c23)cn1] | LC-MS: m/z 439 (M + H)⁺. ¹H NMR (400 MHz, DMSO- d₆) δ: 8.56 (d, J = 1.8 Hz, 1H), 7.84 (dd, J = 7.9, 2.2 Hz, 1H), 7.71 (dd, J = 4.8, 2.9 Hz, 2H), 7.56 (d, J = 8.5 Hz, 1H), 7.39 (d, J = 8.0 Hz, 1H), 7.29 (dd, J = 8.5, 1.9 Hz, 1H), 6.46 (d, J = 3.8 Hz, 1H), 3.74 (s, 3H), 3.35 (d, J = 7.2 Hz, 2H), 2.83 (q, J = 7.6 Hz, 2H), 2.55 (s, 3H), 1.27 (t, J = 7.6 Hz, 3H), 0.70-0.58 (m, 1H), 0.36-0.28 (m, 2H), 0.08-0.04 (m, 2H). |
| | 5-(cyclopropylmethyl)-2-(1,2-dimethyl-1H- | |
| | benzo[d]imidazol-6-yl)-4-(6-ethylpyridin-3-yl)-2,5- | |
| | dihydro-3H-pyrrolo[3,2-c]pyridazin-3-one | |
| 143 | 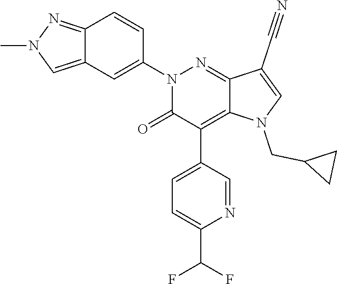 [SMILES: Cn1cc2cc(-n3nc4c(C#N)cn(CC5CC5)c4c(-c4ccc(C(F)F)nc4)c3=O)ccc2n1] | LC-MS: m/z 472 (M + H)⁺. ¹H NMR (400 MHz, DMSO- d₆) δ: 8.69 (s, 1H), 8.60 (s, 1H), 8.32 (s, 1H), 8.05 (dd, J = 8.1, 1.8 Hz, 1H), 7.81 (d, J = 1.4 Hz, 1H), 7.71 (d, J = 8.1 Hz, 1H), 7.54 (d, J = 9.2 Hz, 1H), 7.25 (dd, J = 9.2, 1.9 Hz, 1H), 6.92 (t, J_{HF} = 54.8 Hz, 1H), 4.22 (s, 3H), 3.21 (d, J = 7.2 Hz, 2H), 0.62-0.51 (m, 1H), 0.24-0.17 (m, 2H), 0.03- −0.03 (m, 2H). |
| | 5-(cyclopropylmethyl)-4-(6-(difluoromethyl)pyridin-3- | |
| | yl)-2-(2-methyl-2H-indazol-5-yl)-3-oxo-3,5-dihydro-2H- | |
| | pyrrolo[3,2-c]pyridazine-7-carbonitrile | |
| 144 | 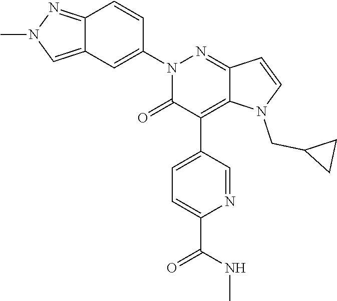 [SMILES: CNC(=O)c1ccc(-c2c(=O)n(-c3ccc4nn(C)cc4c3)nc3ccn(CC4CC4)c23)cn1] | LC-MS: m/z 454 (M + H)⁺. ¹H NMR (400 MHz, DMSO- d₆) δ: 8.88 (q, J = 4.6 Hz, 1H), 8.77 (t, J = 1.4 Hz, 1H), 8.43 (s, 1H), 8.14 (s, 2H), 7.91 (d, J = 1.5 Hz, 1H), 7.73 (d, J = 3.8 Hz, 1H), 7.65 (d, J = 9.1 Hz, 1H), 7.39 (dd, J = 9.1, 2.0 Hz, 1H), 6.49 (d, J = 3.8 Hz, 1H), 4.20 (s, 3H), 3.33 (d, J = 7.2 Hz, 2H), 2.85 (d, J = 4.8 Hz, 3H), 0.69-0.57 (m, 1H), 0.34- 0.26 (m, 2H), 0.12-0.03 (m, 2H). |
| | 5-(5-(cyclopropylmethyl)-2-(2-methyl-2H-indazol-5-yl)- | |
| | 3-oxo-3,5-dihydro-2H-pyrrolo[3,2-c]pyridazin-4-yl)-N- | |
| | methylpicolinamide | |
| 145 | 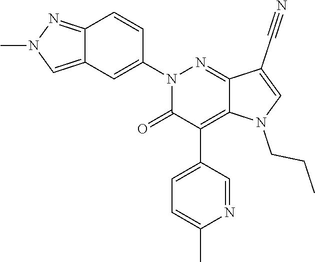 [SMILES: CCCn1cc(C#N)c2nn(-c3ccc4nn(C)cc4c3)c(=O)c(-c3ccc(C)nc3)c21] | LC-MS: m/z 424 (M + H)⁺. ¹H NMR (400 MHz, DMSO- d₆) δ: 8.66 (s, 1H), 8.55 (d, J = 1.6 Hz, 1H), 8.47 (s, 1H), 7.94 (d, J = 1.2 Hz, 1H), 7.85 (dd, J = 8.0, 2.4 Hz, 1H), 7.68 (d, J = 9.2 Hz, 1H), 7.44-7.34 (m, 2H), 4.21 (s, 3H), 3.52-3.47 (m, 2H), 2.55 (s, 3H), 0.80- 0.85 (m, 2H), 0.46 (t, J = 7.2 Hz, 3H). |
| | 2-(2-methyl-2H-indazol-5-yl)-4-(6-methylpyridin-3- | |
| | yl)-3-oxo-5-propyl-3,5-dihydro-2H-pyrrolo[3,2- | |
| | c]pyridazine-7-carbonitrile | |
| 146 | 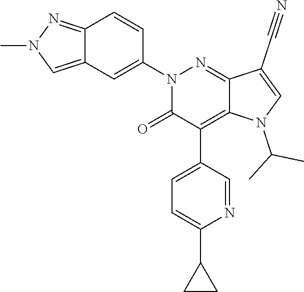 [SMILES: CC(C)n1cc(C#N)c2nn(-c3ccc4nn(C)cc4c3)c(=O)c(-c3ccc(C4CC4)nc3)c21] | LC-MS: m/z 450 (M + H)⁺. ¹H NMR (400 MHz, DMSO- d₆) δ: 8.90 (s, 1H), 8.48 (d, J = 9.2 Hz, 2H), 7.93 (s, 1H), 7.80 (dd, J = 8.0, 2.1 Hz, 1H), 7.68 (d, J = 9.2 Hz, 1H), 7.44 (d, J = 8.0 Hz, 1H), 7.37 (dd, J = 9.2, 2.0 Hz, 1H), 4.22 (s, 3H), 3.86-3.79 (m, 1H), 2.20-2.15 (m, 1H), 1.19 (d, J = 6.4 Hz, 6H), 1.03-0.95 (m, 4H). |
| | 4-(6-cyclopropylpyridin-3-yl)-5-isopropyl-2-(2- | |
| | methyl-2H-indazol-5-yl)-3-oxo-3,5-dihydro-2H- | |
| | pyrrolo[3,2-c]pyridazine-7-carbonitrile | |
| 147 | 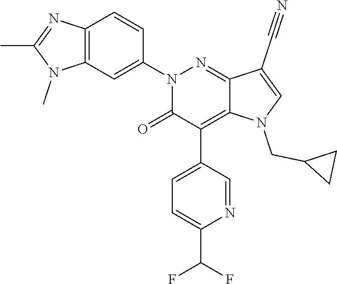 [SMILES: Cc1nc2ccc(-n3nc4c(C#N)cn(CC5CC5)c4c(-c4ccc(C(F)F)nc4)c3=O)cc2n1C] | LC-MS: m/z 486 (M + H)⁺. ¹H NMR (400 MHz, DMSO- d₆) δ: 8.85 (s, 1H), 8.76 (s, 1H), 8.21 (dd, J = 8.0, 2.0 Hz, 1H), 7.86 (d, J = 8.1 Hz, 1H), 7.73 (d, J = 1.9 Hz, 1H), 7.62 (d, J = 8.5 Hz, 1H), 7.31 (dd, J = 8.5, 2.0 Hz, 1H), 7.07 (t, J_{HF} = 54.9 Hz, 1H), 3.76 (s, 3H), 3.38 (d, J = 7.2 Hz, 2H), 2.56 (s, 3H), 0.70-0.56 (m, 1H), 0.39-0.31 (m, 2H), 0.16-0.08 (m, 2H). |
| | 5-(cyclopropylmethyl)-4-(6-(difluoromethyl)pyridin-3- | |
| | yl)-2-(1,2-dimethyl-1H-benzo[d]imidazol-6-yl)-3-oxo- | |
| | 3,5-dihydro-2H-pyrrolo[3,2-c]pyridazine-7-carbonitrile | |
| 148 | 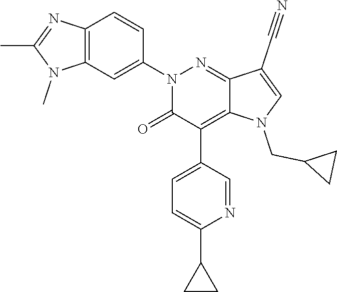 [SMILES: Cc1nc2ccc(-n3nc4c(C#N)cn(CC5CC5)c4c(-c4ccc(C5CC5)nc4)c3=O)cc2n1C] | LC-MS: m/z 476 (M + H)⁺. ¹H NMR (400 MHz, DMSO- d₆) δ: 8.73 (s, 1H), 8.50 (d, J = 1.9 Hz, 1H), 7.80 (dd, J = 8.0, 2.2 Hz, 1H), 7.72 (d, J = 1.9 Hz, 1H), 7.60 (d, J = 8.5 Hz, 1H), 7.44 (d, J = 8.1 Hz, 1H), 7.29 (dd, J = 8.5, 2.0 Hz, 1H), 3.75 (s, 3H), 3.39 (d, J = 7.2 Hz, 2H), 2.56 (s, 3H), 2.20- 2.16 (m, 1H), 1.04-0.94 (m, 4H), 0.80-0.58 (m, 1H), 0.38- 0.30 (m, 2H), 0.15-0.07 (m, 2H). |
| | 5-(cyclopropylmethyl)-4-(6-cyclopropylpyridin-3-yl)-2- | |
| | (1,2-dimethyl-1H-benzo[d]imidazol-6-yl)-3-oxo-3,5- | |
| | dihydro-2H-pyrrolo[3,2-c]pyridazine-7-carbonitrile | |
| 149 | 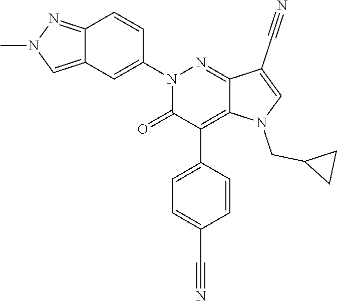 [SMILES: Cn1cc2cc(-n3nc4c(C#N)cn(CC5CC5)c4c(-c4ccc(C#N)cc4)c3=O)ccc2n1] | LC-MS: m/z 446 (M + H)⁺. ¹H NMR (400 MHz, DMSO- d₆) δ: 8.73 (s, 1H), 8.47 (s, 1H), 7.99 (d, J = 8.2 Hz, 2H), 7.95 (d, J = 1.4 Hz, 1H), 7.75 (d, J = 8.2 Hz, 2H), 7.68 (d, J = 9.1 Hz, 1H), 7.39 (dd, J = 9.2, 1.9 Hz, 1H), 4.21 (s, 3H), 3.32 (d, J = 7.3 Hz, 2H), 0.77- 0.62 (m, 1H), 0.38-0.33 (m, 2H), 0.15-0.10 (m, 2H). |
| | 4-(4-cyanophenyl)-5-(cyclopropylmethyl)-2-(2-methyl- | |
| | 2H-indazol-5-yl)-3-oxo-3,5-dihydro-2H-pyrrolo[3,2- | |
| | c]pyridazine-7-carbonitrile | |
| 150 | 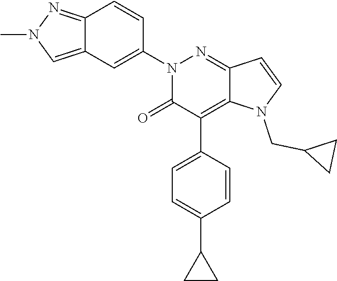 [SMILES: Cn1cc2cc(-n3nc4ccn(CC5CC5)c4c(-c4ccc(C5CC5)cc4)c3=O)ccc2n1] | LC-MS: m/z 436 (M + H)⁺. ¹H NMR (400 MHz, DMSO- d₆) δ: 8.42 (s, 1H), 7.88 (d, J = 1.6 Hz, 1H), 7.67 (d, J = 3.8 Hz, 1H), 7.63 (d, J = 9.1 Hz, 1H), 7.36 (dd, J = 9.2, 2.0 Hz, 1H), 7.33 (d, J = 8.2 Hz, 2H), 7.17 (d, J = 8.2 Hz, 2H), 6.41 (d, J = 3.7 Hz, 1H), 4.20 (s, 3H), 3.32 (d, J = 7.2 Hz, 2H), 2.04-1.95 (m, 1H), 1.05-0.98 (m, 2H), 0.77-0.72 (m, 2H), 0.71-0.64 (m, 1H), 0.31-0.24 (m, 2H), 0.07-0.01 (m, 2H). |
| | 5-(cyclopropylmethyl)-4-(4-cyclopropylphenyl)-2-(2- | |
| | methyl-2H-indazol-5-yl)-2,5-dihydro-3H-pyrrolo[3,2- | |
| | c]pyridazin-3-one | |
| 151 | 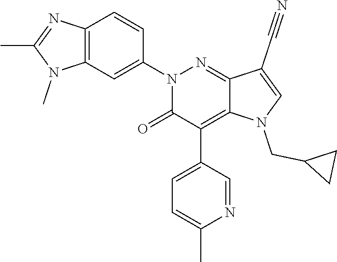 [SMILES: Cc1ccc(-c2c(=O)n(-c3ccc4nc(C)n(C)c4c3)nc3c(C#N)cn(CC4CC4)c23)cn1] | LC-MS: m/z 450 (M + H)⁺. ¹H NMR (400 MHz, DMSO- d₆) δ: 8.73 (s, 1H), 8.56 (d, J = 2.0 Hz, 1H), 7.85 (dd, J = 7.9, 2.3 Hz, 1H), 7.72 (d, J = 1.9 Hz, 1H), 7.60 (d, J = 8.5 Hz, 1H), 7.40 (d, J = 8.0 Hz, 1H), 7.29 (dd, J = 8.5, 2.0 Hz, 1H), 3.75 (s, 3H), 3.40 (d, J = 7.1 Hz, 2H), 2.56 (s, 3H), 2.55 (s, 3H), 0.80-0.68 (m, 1H), 0.39- 0.33 (m, 2H), 0.18-0.10 (m, 2H). |
| | 5-(cyclopropylmethyl)-2-(1,2-dimethyl-1H- | |
| | benzo[d]imidazol-6-yl)-4-(6-methylpyridin-3-yl)-3- | |
| | oxo-3,5-dihydro-2H-pyrrolo[3,2-c]pyridazine-7- | |
| | carbonitrile | |
| 152 | 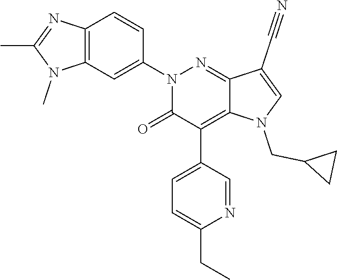 [SMILES: CCc1ccc(-c2c(=O)n(-c3ccc4nc(C)n(C)c4c3)nc3c(C#N)cn(CC4CC4)c23)cn1] | LC-MS: m/z 464 (M + H)⁺. ¹H NMR (400 MHz, DMSO- d₆) δ: 8.72 (s, 1H), 8.59 (d, J = 1.9 Hz, 1H), 7.87 (dd, J = 8.0, 2.3 Hz, 1H), 7.73 (d, J = 1.9 Hz, 1H), 7.61 (d, J = 8.5 Hz, 1H), 7.41 (d, J = 8.0 Hz, 1H), 7.30 (dd, J = 8.5, 2.0 Hz, 1H), 3.75 (s, 3H), 3.39 (d, J = 7.2 Hz, 2H), 2.83 (q, J = 7.5 Hz, 2H), 2.56 (s, 3H), 1.27 (t, J = 7.6 Hz, 3H), 0.75-0.64 (m, 1H), 0.41-0.32 (m, 2H), 0.16- 0.09 (m, 2H). |
| | 5-(cyclopropylmethyl)-2-(1,2-dimethyl-1H- | |
| | benzo[d]imidazol-6-yl)-4-(6-ethylpyridin-3-yl)-3-oxo- | |
| | 3,5-dihydro-2H-pyrrolo[3,2-c]pyridazine-7-carbonitrile | |
| 153 | 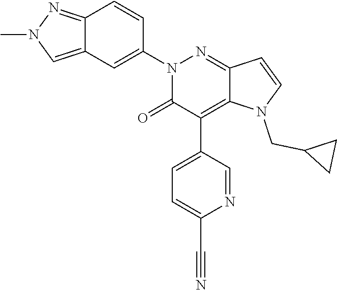 [SMILES: Cn1cc2cc(-n3nc4ccn(CC5CC5)c4c(-c4ccc(C#N)nc4)c3=O)ccc2n1] | LC-MS: m/z 422 (M + H)⁺. ¹H NMR (400 MHz, DMSO- d₆) δ: 8.92 (d, J = 1.4 Hz, 1H), 8.43 (s, 1H), 8.26 (dd, J = 8.0, 2.0 Hz, 1H), 8.18 (d, J = 8.0 Hz, 1H), 7.90 (d, J = 1.6 Hz, 1H), 7.75 (d, J = 3.8 Hz, 1H), 7.65 (d, J = 9.2 Hz, 1H), 7.38 (dd, J = 9.1, 2.0 Hz, 1H), 6.51 (d, J = 3.8 Hz, 1H), 4.20 (s, 3H), 3.31 (d, J = 7.2 Hz, 2H), 0.59-0.68 (m, 1H), 0.36-0.30 (m, 2H), 0.14-0.09 (m, 2H). |
| | 5-(5-(cyclopropylmethyl)-2-(2-methyl-2H-indazol-5-yl)- | |
| | 3-oxo-3,5-dihydro-2H-pyrrolo[3,2-c]pyridazin-4- | |
| | yl)picolinonitrile | |
| 154 | 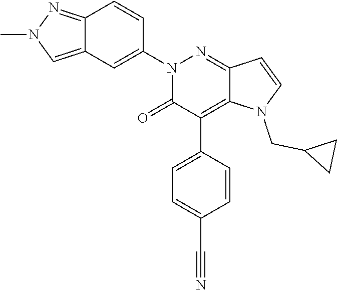 [SMILES: Cn1cc2cc(-n3nc4ccn(CC5CC5)c4c(-c4ccc(C#N)cc4)c3=O)ccc2n1] | LC-MS: m/z 421 (M + H)⁺. ¹H NMR (400 MHz, DMSO- d₆) δ: 8.43 (s, 1H), 7.96 (d, J = 8.3 Hz, 2H), 7.90 (d, J = 1.4 Hz, 1H), 7.74 (s, 1H), 7.71 (d, J = 3.4 Hz, 2H), 7.64 (d, J = 9.1 Hz, 1H), 7.37 (dd, J = 9.1, 2.0 Hz, 1H), 6.47 (d, J = 3.8 Hz, 1H), 4.20 (s, 3H), 3.28 (d, J = 7.1 Hz, 2H), 0.70-0.62 (m, 1H), 0.33-0.27 (m, 2H), 0.10- 0.05 (m, 2H). |
| | 4-(5-(cyclopropylmethyl)-2-(2-methyl-2H-indazol-5-yl)- | |
| | 3-oxo-3,5-dihydro-2H-pyrrolo[3,2-c]pyridazin-4- | |
| | yl)benzonitrile | |
| 155 | 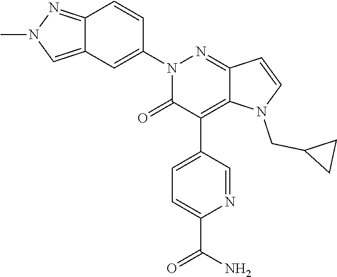 [SMILES: Cn1cc2cc(-n3nc4ccn(CC5CC5)c4c(-c4ccc(C(N)=O)nc4)c3=O)ccc2n1] | LC-MS: m/z 440 (M + H)⁺. ¹H NMR (400 MHz, DMSO- d₆) δ: 8.76 (t, J = 1.4 Hz, 1H), 8.43 (s, 1H), 8.22 (s, 1H), 8.14 (d, J = 1.4 Hz, 2H), 7.91 (d, J = 1.3 Hz, 1H), 7.73 (d, J = 3.8 Hz, 2H), 7.64 (d, J = 9.1 Hz, 1H), 7.38 (dd, J = 9.1, 2.0 Hz, 1H), 6.49 (d, J = 3.8 Hz, 1H), 4.20 (s, 3H), 3.29 (d, J = 7.1 Hz, 2H), 0.70-0.58 (m, 1H), 0.35-0.24 (m, 2H), 0.13- 0.03 (m, 2H). |
| | 5-(5-(cyclopropylmethyl)-2-(2-methyl-2H-indazol-5-yl)- | |
| | 3-oxo-3,5-dihydro-2H-pyrrolo[3,2-c]pyridazin-4- | |
| | yl)picolinamide | |
| 156 | 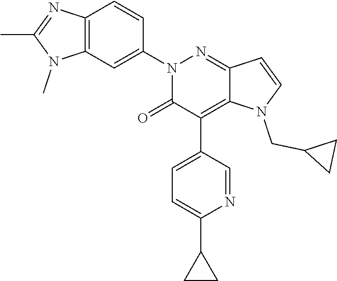 [SMILES: Cc1nc2ccc(-n3nc4ccn(CC5CC5)c4c(-c4ccc(C5CC5)nc4)c3=O)cc2n1C] | LC-MS: m/z 451 (M + H)⁺. ¹H NMR (400 MHz, DMSO- d₆) δ: 8.47 (d, J = 1.8 Hz, 1H), 7.77 (dd, J = 8.0, 2.2 Hz, 1H), 7.70 (dd, J = 4.2, 2.9 Hz, 2H), 7.56 (d, J = 8.4 Hz, 1H), 7.41 (d, J = 8.0 Hz, 1H), 7.28 (dd, J = 8.5, 1.9 Hz, 1H), 6.44 (d, J = 3.8 Hz, 1H), 3.74 (s, 3H), 3.30 (d, J = 7.2 Hz, 2H), 2.55 (s, 3H), 2.24-2.15 (m, 1H), 1.00-0.92 (m, 4H), 0.70-0.58 (m, 1H), 0.36-0.27 (m, 2H), 0.12-0.05 (m, 2H). |
| | 5-(cyclopropylmethyl)-4-(6-cyclopropylpyridin-3-yl)-2- | |
| | (1,2-dimethyl-1H-benzo[d]imidazol-6-yl)-2,5-dihydro- | |
| | 3H-pyrrolo[3,2-c]pyridazin-3-one | |
| 157 | 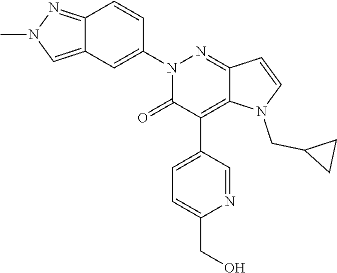 [SMILES: Cn1cc2cc(-n3nc4ccn(CC5CC5)c4c(-c4ccc(CO)nc4)c3=O)ccc2n1] | LC-MS: m/z 427 (M + H)⁺. ¹H NMR (400 MHz, DMSO- d₆) δ: 8.55 (d, J = 1.4 Hz, 1H), 8.44 (s, 1H), 7.97-7.85 (m, 2H), 7.73 (d, J = 3.7 Hz, 1H), 7.61 (dd, J = 18.2, 8.6 Hz, 2H), 7.37 (dd, J = 9.1, 1.9 Hz, 1H), 6.45 (d, J = 3.7 Hz, 1H), 5.66 (t, J = 5.4 Hz, 1H), 4.63 (d, J = 5.6 Hz, 2H), 4.19 (s, 3H), 3.34 (d, J = 7.1 Hz, 2H), 0.68-0.63 (m, 1H), 0.30-0.25 (m, 2H), 0.10-0.05 (m, 2H). |
| | 5-(cyclopropylmethyl)-4-(6-(hydroxymethyl)pyridin-3- | |
| | yl)-2-(2-methyl-2H-indazol-5-yl)-2,5-dihydro-3H- | |
| | pyrrolo[3,2-c]pyridazin-3-one | |
| 158 | 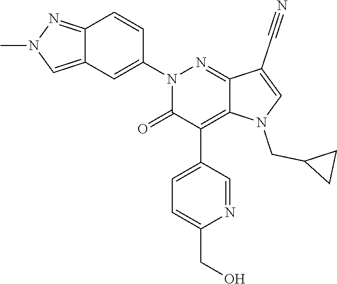 [SMILES: Cn1cc2cc(-n3nc4c(C#N)cn(CC5CC5)c4c(-c4ccc(CO)nc4)c3=O)ccc2n1] | LC-MS: m/z 452 (M + H)⁺. ¹H NMR (400 MHz, DMSO- d₆) δ: 8.74 (s, 1H), 8.59 (d, J = 1.7 Hz, 1H), 8.47 (s, 1H), 8.01-7.92 (m, 2H), 7.68 (d, J = 9.1 Hz, 1H), 7.60 (d, J = 8.1 Hz, 1H), 7.39 (dd, J = 9.2, 2.0 Hz, 1H), 5.57 (t, J = 5.9 Hz, 1H), 4.64 (d, J = 5.8 Hz, 2H), 4.21 (s, 3H), 3.39 (d, J = 7.2 Hz, 2H), 0.80-0.68 (m, 1H), 0.40-0.33 (m, 2H), 0.19- 0.11 (m, 2H). |
| | 5-(cyclopropylmethyl)-4-(6-(hydroxymethyl)pyridin-3- | |
| | yl)-2-(2-methyl-2H-indazol-5-yl)-3-oxo-3,5-dihydro- | |
| | 2H-pyrrolo[3,2-c]pyridazine-7-carbonitrile | |
| 159 | 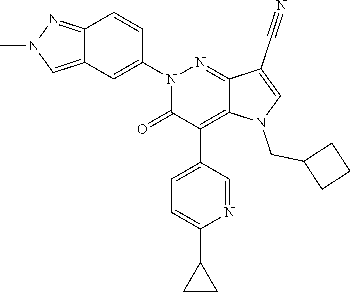 [SMILES: Cn1cc2cc(-n3nc4c(C#N)cn(CC5CCC5)c4c(-c4ccc(C5CC5)nc4)c3=O)ccc2n1] | LC-MS: m/z 476 (M + H)⁺. ¹H NMR (400 MHz, DMSO- d₆) δ: 8.65 (s, 1H), 8.49 (d, J = 2.0 Hz, 1H), 8.46 (s, 1H), 7.93 (d, J = 1.6 Hz, 1H), 7.79 (dd, J = 8.0, 2.0 Hz, 1H), 7.68 (d, J = 9.2 Hz, 1H), 7.44 (d, J = 8.0 Hz, 1H), 7.38 (dd, J = 9.0, 2.0 Hz, 1H), 4.21 (s, 3H), 3.55 (d, J = 7.6 Hz, 2H), 2.20-2.16 (m, 2H), 1.69-1.61 (m, 4H), 1.48- 1.39 (m, 2H), 1.01-0.92 (m, 4H). |
| | 5-(cyclobutylmethyl)-4-(6-cyclopropylpyridin-3-yl)-2-(2- | |
| | methyl-2H-indazol-5-yl)-3-oxo-3,5-dihydro-2H-pyrrolo[3,2- | |
| | c]pyridazine-7-carbonitrile | |

The procedure set forth above for General Procedure II (Method B) was used to synthesize the following compounds by using appropriate starting materials:

| Example | Structure | Characterization |
|---|---|---|
| 160 | 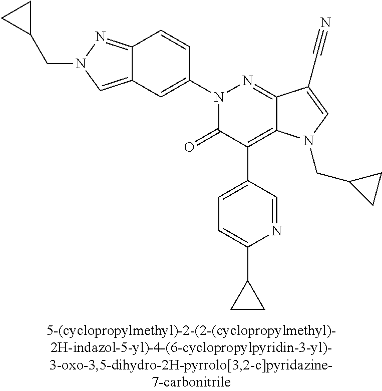 [SMILES: N#Cc1cn(CC2CC2)c2c(-c3ccc(C4CC4)nc3)c(=O)n(-c3ccc4nn(CC5CC5)cc4c3)nc12] | LC-MS: m/z 502 (M + H)⁺. ¹H NMR (400 MHz, DMSO- d₆) δ: 8.72 (s, 1H), 8.54 (s, 1H), 8.50 (d, J = 1.8 Hz, 1H), 7.95 (d, J = 1.4 Hz, 1H), 7.81 (dd, J = 8.0, 2.2 Hz, 1H), 7.70 (d, J = 9.2 Hz, 1H), 7.44 (d, J = 8.0 Hz, 1H), 7.38 (dd, J = 9.2, 2.0 Hz, 1H), 4.33 (d, J = 7.2 Hz, 2H), 3.39 (d, J = 7.2 Hz, 2H), 2.24-2.14 (m, 1H), 1.48-1.35 (m, 1H), 1.04-0.94 (m, 4H), 0.77-0.67 (m, 1H), 0.61-0.55 (m, 2H), 0.49-0.44 (m, 2H), 0.39-0.33 (m, 2H), 0.17-0.12 (m, 2H). |
| 161 | 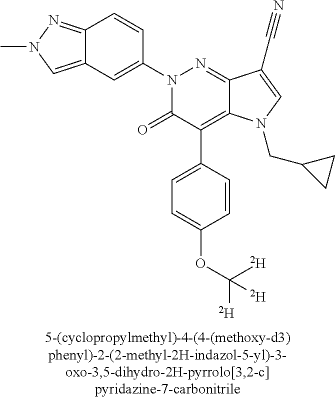 [SMILES: [2H]C([2H])([2H])Oc1ccc(-c2c(=O)n(-c3ccc4nn(C)cc4c3)nc3c(C#N)cn(CC4CC4)c23)cc1] | LC-MS: m/z 454 (M + H)⁺. ¹H NMR (400 MHz, DMSO- d₆) δ: 8.68 (s, 1H), 8.46 (s, 1H), 7.93 (d, J = 1.6 Hz, 1H), 7.67 (d, J = 9.2 Hz, 1H), 7.52- 7.40 (m, 2H), 7.38 (dd, J = 9.2, 2.0 Hz, 1H), 7.05 (d, J = 8.8 Hz, 2H), 4.21 (s, 3H), 3.39 (d, J = 7.4 Hz, 2H), 0.76-0.72 (m, 1H), 0.45-0.25 (m, 2H), 0.14-0.09 (m, 2H). |

### Example 162: Synthesis of 5-(cyclopropylmethyl)-4-(6-cyclopropylpyridin-3-yl)-N-methyl-2-(2-methyl-2H-indazol-5-yl)-3-oxo-2H,3H,5H-pyrrolopyridazine-7-carboxamide via General Procedure III

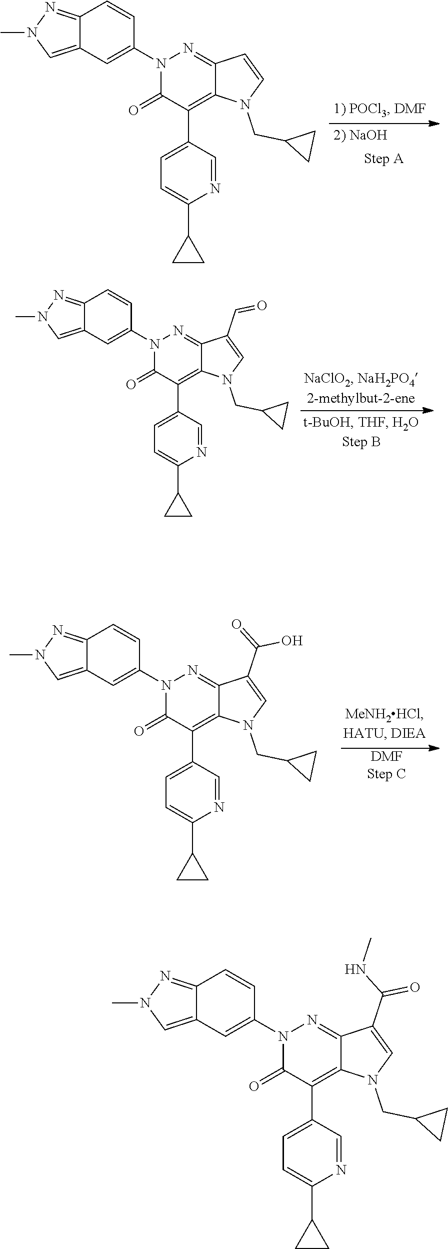 [SMILES: Cn1cc2cc(-n3nc4ccn(CC5CC5)c4c(-c4ccc(C5CC5)nc4)c3=O)ccc2n1 ; Cn1cc2cc(-n3nc4c(C=O)cn(CC5CC5)c4c(-c4ccc(C5CC5)nc4)c3=O)ccc2n1 ; Cn1cc2cc(-n3nc4c(C(=O)O)cn(CC5CC5)c4c(-c4ccc(C5CC5)nc4)c3=O)ccc2n1 ; "DMFStep C" ; CNC(=O)c1cn(CC2CC2)c2c(-c3ccc(C4CC4)nc3)c(=O)n(-c3ccc4nn(C)cc4c3)nc12]

### Step A: 5-(cyclopropylmethyl)-4-(6-cyclopropylpyridin-3-yl)-2-(2-methyl-2H-indazol-5-yl)-3-oxo-3,5-dihydro-2H-pyrrolo[3,2-c]pyridazine-7-carbaldehyde

To a solution of 5-(cyclopropylmethyl)-4-(6-cyclopropylpyridin-3-yl)-2-(2-methyl-2H-indazol-5-yl)-2H,3H,5H-pyrrolopyridazin-3-one (Example 106) (3.63 g, 8.3 mmol, 1.0 eq.) in dry DMF (37 mL) was added POCl₃ (5.10 g, 33.3 mmol, 4.0 eq.) at 0° C. The reaction mixture was stirred at rt for 15 hrs, then NaOH (1N, aq.) (100 mL) was added into the mixture at 0° C. The reaction mixture was stirred at rt for an additional 3 hrs until LCMS showed reaction completion. The resulting mixture was poured into ice-cooled NaHCO₃(sat. aq.) (15 mL), extracted with DCM (100 mL×3), washed with brine (50 mL), dried over Na₂SO₄, and concentrated under reduced pressure. The resulting residue was purified by flash column chromatography on silica gel to give 5-(cyclopropylmethyl)-4-(6-cyclopropylpyridin-3-yl)-2-(2-methyl-2H-indazol-5-yl)-3-oxo-2H,3H,5H-pyrrolopyridazine-7-carbaldehyde (3.29 g, 85%) as a yellow solid. LC-MS (ESI): m/z 465 [M+H]⁺.

### Step B: 5-(cyclopropylmethyl)-4-(6-cyclopropylpyridin-3-yl)-2-(2-methyl-2H-indazol-5-yl)-3-oxo-3,5-dihydro-2H-pyrrolo[3,2-c]pyridazine-7-carboxylic Acid

To a solution of 5-(cyclopropylmethyl)-4-(6-cyclopropylpyridin-3-yl)-2-(2-methyl-2H-indazol-5-yl)-3-oxo-2H,3H,5H-pyrrolopyridazine-7-carbaldehyde (10.0 g, 21.5 mmol, 1.0 eq.) in THE (100 mL), t-BuOH (100 mL) and H₂O (50 mL) was added NaClO₂ (19.4 g, 215.2 mmol, 10.0 eq.), NaH₂PO₄ (20.7 g, 172.2 mmol, 8.0 eq.) and 2-methylbut-2-ene (30.2 g, 430.5 mmol, 20.0 eq.). The reaction mixture was stirred at room temperature overnight, causing a precipitation to form. The reaction was determined to be complete by TLC (DCM:MeOH=10:1), then the reaction mixture was filtered, the filter cake was washed with 50 mL of water, and the filter cake was dried under vacuum to afford 5-(cyclopropylmethyl)-4-(6-cyclopropylpyridin-3-yl)-2-(2-methyl-2H-indazol-5-yl)-3-oxo-2H,3H,5H-pyrrolopyridazine-7-carboxylic acid (8.1 g, 78% yield) as a yellow solid. LC-MS (ESI): m/z 481 [M+H]⁺.

### Step C: 5-(cyclopropylmethyl)-4-(6-cyclopropylpyridin-3-yl)-N-methyl-2-(2-methyl-2H-indazol-5-yl)-3-oxo-3,5-dihydro-2H-pyrrolo[3,2-c]pyridazine-7-carboxamide

To a solution of 5-(cyclopropylmethyl)-4-(6-cyclopropylpyridin-3-yl)-2-(2-methyl-2H-indazol-5-yl)-3-oxo-2H,3H,5H-pyrrolopyridazine-7-carboxylic acid (50 mg, 104 μmol, 1.0 eq.) in DMF (1.5 mL) was added MeNH₂.HCl (8 mg, 125 μmol, 1.2 eq.), HATU (41 mg, 109 μmol, 1.05 eq.) and DIEA (40 mg, 312 μmol, 3.0 eq.), the reaction mixture was stirred at room temperature for 30 min, the reaction was completed as indicated by TLC. The reaction mixture was diluted with H₂O (20 mL), extracted with DCM (20 mL×3). The combined organic layers were washed with brine (10 mL) and dried over anhydrous Na₂SO₄, concentrated under reduced pressure, the residue was purified by RP-Prep-HPLC to afford 5-(cyclopropylmethyl)-4-(6-cyclopropylpyridin-3-yl)-N-methyl-2-(2-methyl-2H-indazol-5-yl)-3-oxo-2H,3H,5H-pyrrolopyridazine-7-carboxamide. ¹H NMR (400 MHz, DMSO-d₆) δ 8.45 (s, 1H), 8.30 (s, 1H), 7.98 (d, J=1.5 Hz, 1H), 7.79 (dd, J=8.0, 2.2 Hz, 1H), 7.67 (d, J=9.2 Hz, 1H), 7.45-7.38 (m, 3H), 4.21 (s, 3H), 3.40 (s, 2H), 2.79 (d, J=4.8 Hz, 3H), 2.18 (t, J=4.9 Hz, 1H), 2.07 (s, 1H), 1.03-0.93 (m, 4H), 0.69 (m, 1H), 0.34-0.26 (m, 2H), 0.11 (q, J=4.8 Hz, 2H). LC-MS (ESI): m/z 494 [M+H]⁺.

The procedure set forth above for General Procedure III was used to synthesize the following compounds by using appropriate starting materials:

| Example | Structure | Characterization |
|---|---|---|
| 163 (From Step B) | 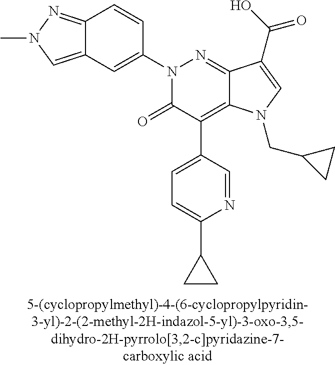 [SMILES: Cn1cc2cc(-n3nc4c(C(=O)O)cn(CC5CC5)c4c(-c4ccc(C5CC5)nc4)c3=O)ccc2n1] | LC-MS: m/z 481 (M + H)⁺. ¹H NMR (400 MHz, DMSO- d₆) δ: 8.48 (s, 1H), 8.47 (s, 1H), 8.12 (s, 1H), 7.87 (s, 1H), 7.77 (dd, J = 8.0, 2.1 Hz, 1H), 7.64 (d, J = 9.2 Hz, 1H), 7.41 (d, J = 8.1 Hz, 1H), 7.35 (dd, J = 9.1, 1.8 Hz, 1H), 4.20 (s, 3H), 3.30 (s, 2H), 2.22- 2.12 (m, 1H), 1.07-0.90 (m, 4H), 0.76-0.62 (m, 1H), 0.33- 0.30 (m, 2H), 0.10-0.05 (m, 2H). |
| 164 | 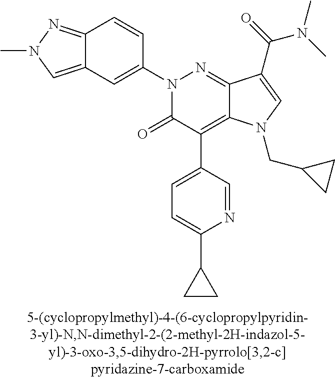 [SMILES: CN(C)C(=O)c1cn(CC2CC2)c2c(-c3ccc(C4CC4)nc3)c(=O)n(-c3ccc4nn(C)cc4c3)nc12] | LC-MS: m/z 508 (M + H)⁺. ¹H NMR (400 MHz, DMSO- d₆) δ: 8.49 (d, J = 1.7 Hz, 1H), 8.44 (s, 1H), 8.03 (s, 1H), 7.89 (d, J = 1.3 Hz, 1H), 7.79 (dd, J = 8.0, 2.2 Hz, 1H), 7.65 (d, J = 9.1 Hz, 1H), 7.42 (d, J = 8.0 Hz, 1H), 7.36 (dd, J = 9.1, 1.9 Hz, 1H), 4.20 (s, 3H), 3.31 (s, 2H), 3.01 (s, 6H), 2.25-2.12 (m, 1H), 1.06-0.91 (m, 4H), 0.77-0.64 (m, 1H), 0.33-0.30 (m, 2H), 0.11-0.06 (m, 2H). |
| 165 | 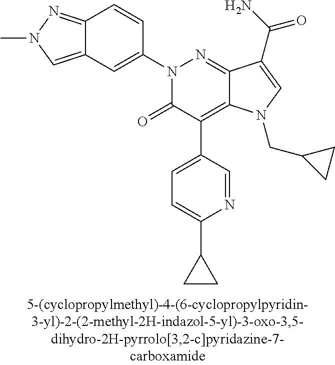 [SMILES: Cn1cc2cc(-n3nc4c(C(N)=O)cn(CC5CC5)c4c(-c4ccc(C5CC5)nc4)c3=O)ccc2n1] | LC-MS: m/z 480 (M + H)⁺. ¹H NMR (400 MHz, DMSO- d₆) δ: 8.50 (d, J = 1.8 Hz, 1H), 8.45 (s, 1H), 8.31 (s, 1H), 7.98 (d, J = 1.3 Hz, 1H), 7.81 (dd, J = 8.0, 2.2 Hz, 1H), 7.67 (d, J = 9.1 Hz, 1H), 7.48 (s, 1H), 7.45-7.41 (m, 2H), 7.02 (s, 1H), 4.20 (s, 3H), 3.39 (d, J = 7.1 Hz, 2H), 2.20-2.17 (m, 1H), 1.04-0.96 (m, 4H), 0.75- 0.66 (m, 1H), 0.36-0.30 (m, 2H), 0.13-0.09 (m, 2H). |
| 166 | 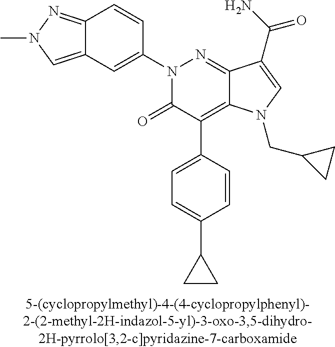 [SMILES: Cn1cc2cc(-n3nc4c(C(N)=O)cn(CC5CC5)c4c(-c4ccc(C5CC5)cc4)c3=O)ccc2n1] | LC-MS: m/z 479 (M + H)⁺. ¹H NMR (400 MHz, DMSO- d₆) δ: 8.45 (s, 1H), 8.29 (s, 1H), 7.97 (d, J = 1.4 Hz, 1H), 7.66 (d, J = 9.2 Hz, 1H), 7.46 (s, 1H), 7.42 (dd, J = 9.2, 2.0 Hz, 1H), 7.36 (d, J = 8.2 Hz, 2H), 7.19 (d, J = 8.2 Hz, 2H), 7.02 (s, 1H), 4.20 (s, 3H), 3.37 (d, J = 7.2 Hz, 2H), 2.04- 1.96 (m, 1H), 1.05-0.98 (m, 2H), 0.77-0.70 (m, 3H), 0.33- 0.26 (m, 2H), 0.10-0.06 (m, 2H). |
| 167 | 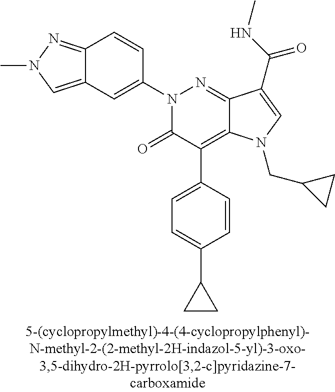 [SMILES: CNC(=O)c1cn(CC2CC2)c2c(-c3ccc(C4CC4)cc3)c(=O)n(-c3ccc4nn(C)cc4c3)nc12] | LC-MS: m/z 493 (M + H)⁺. ¹H NMR (400 MHz, DMSO- d₆) δ: 8.38 (s, 1H), 8.21 (s, 1H), 7.90 (d, J = 1.3 Hz, 1H), 7.59 (d, J = 9.2 Hz, 1H), 7.38- 7.26 (m, 4H), 7.11 (d, J = 8.4 Hz, 2H), 4.13 (s, 3H), 3.27 (d, J = 7.2 Hz, 2H), 2.72 (d, J = 4.8 Hz, 3H), 1.95-1.90 (m, 1H), 0.97-0.91 (m, 2H), 0.67- 0.65 (m, 3H), 0.23-0.20 (m, 2H), −0.01-0.13 (m, 2H). |
| 168 | 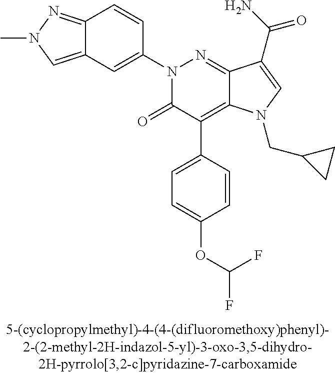 [SMILES: Cn1cc2cc(-n3nc4c(C(N)=O)cn(CC5CC5)c4c(-c4ccc(OC(F)F)cc4)c3=O)ccc2n1] | LC-MS: m/z 505 (M + H)⁺. ¹H NMR (400 MHz, DMSO- d₆) δ: 8.35 (s, 1H), 8.21 (s, 1H), 7.88 (dd, J = 2.0, 0.8 Hz, 1H), 7.57 (d, J = 9.2 Hz, 1H), 7.49-7.45 (m, 2H), 7.38 (s, 1H), 7.33 (dd, J = 9.2, 2.0 Hz, 1H), 7.26 (t, J_{HF} = 74 Hz, 1H), 7.20 (d, J = 8.4 Hz, 2H), 6.93 (s, 1H), 4.10 (s, 3H), 3.27 (d, J = 7.2 Hz, 2H), 0.62-0.56 (m, 1H), 0.25-0.19 (m, 2H), 0.02- −0.03 (m, 2H). |
| 169 | 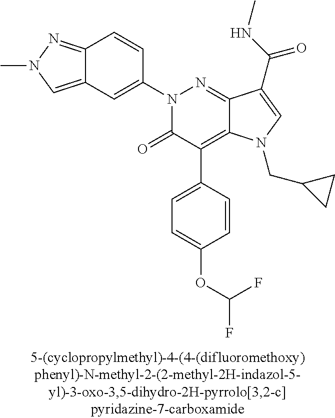 [SMILES: CNC(=O)c1cn(CC2CC2)c2c(-c3ccc(OC(F)F)cc3)c(=O)n(-c3ccc4nn(C)cc4c3)nc12] | LC-MS: m/z 519 (M + H)⁺. ¹H NMR (400 MHz, DMSO- d₆) δ: 8.36 (s, 1H), 8.21 (s, 1H), 7.89 (d, J = 1.6 Hz, 1H), 7.57 (d, J = 9.2 Hz, 1H), 7.50- 7.45 (m, 2H), 7.34 (dd, J = 9.2, 2.0 Hz, 1H), 7.30 (d, J = 4.8 Hz, 1H), 7.26 (t, J_{HF} = 74 Hz, 1H), 7.20 (d, J = 8.8 Hz, 2H), 4.11 (s, 3H), 3.28 (d, J = 7.2 Hz, 2H), 2.70 (d, J = 4.8 Hz, 3H), 0.66-0.56 (m, 1H), 0.25-0.18 (m, 2H), −0.02- 0.01 (m, 2H). |
| 170 | 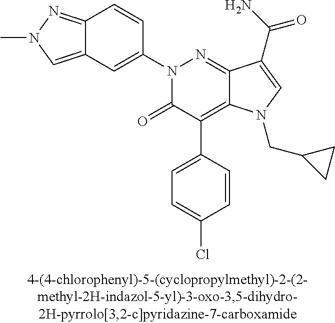 [SMILES: Cn1cc2cc(-n3nc4c(C(N)=O)cn(CC5CC5)c4c(-c4ccc(Cl)cc4)c3=O)ccc2n1] | LC-MS: m/z 473 (M + H)⁺. ¹H NMR (400 MHz, DMSO- d₆) δ: 8.33 (s, 1H), 8.19 (s, 1H), 8.09 (s, 1H), 7.86 (d, J = 1.6 Hz, 1H), 7.54 (d, J = 9.2 Hz, 1H), 7.46-7.41 (m, 4H), 7.36 (s, 1H), 7.31 (dd, J = 9.2, 2.0 Hz, 1H), 6.90 (s, 1H), 4.08 (s, 3H), 3.26 (d, J = 7.2 Hz, 2H), 0.64-0.55 (m, 1H), 0.24- 0.17 (m, 2H), −0.02-0.01 (m, 2H). |
| 171 | 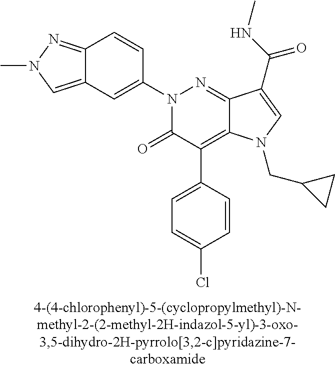 [SMILES: CNC(=O)c1cn(CC2CC2)c2c(-c3ccc(Cl)cc3)c(=O)n(-c3ccc4nn(C)cc4c3)nc12] | LC-MS: m/z 487 (M + H)⁺. ¹H NMR (400 MHz, DMSO- d₆) δ: 8.46 (s, 1H), 8.31 (s, 1H), 7.99 (d, J = 1.4 Hz, 1H), 7.67 (d, J = 9.1 Hz, 1H), 7.62- 7.46 (m, 4H), 7.44 (dd, J = 9.2, 2.0 Hz, 1H), 7.42-7.34 (m, 1H), 4.21 (s, 3H), 3.39 (d, J = 7.2 Hz, 2H), 2.80 (d, J = 4.8 Hz, 3H), 0.84-0.67 (m, 1H), 0.40-0.24 (m, 2H), 0.14- 0.10 (m, 2H). |
| 172 | 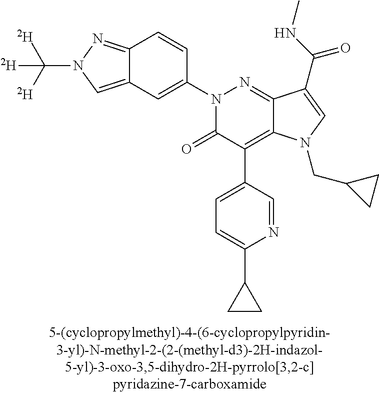 [SMILES: [2H]C([2H])([2H])n1cc2cc(-n3nc4c(C(=O)NC)cn(CC5CC5)c4c(-c4ccc(C5CC5)nc4)c3=O)ccc2n1] | LC-MS: m/z 497 (M + H)⁺. ¹H NMR (400 MHz, DMSO- d₆) δ: 8.50 (d, J = 1.4 Hz, 1H), 8.46 (s, 1H), 8.31 (s, 1H), 7.99 (d, J = 1.0 Hz, 1H), 7.80 (dd, J = 8.0, 2.0 Hz, 1H), 7.67 (d, J = 9.1 Hz, 1H), 7.51-7.34 (m, 3H), 3.39 (d, J = 7.0 Hz, 2H), 2.80 (d, J = 4.7 Hz, 3H), 2.29-2.10 (m, 1H), 1.14-0.88 (m, 4H), 0.70-0.59 (m, 1H), 0.33-0.30 (m, 2H), 0.12-0.06 (m, 2H). |
| 173 | 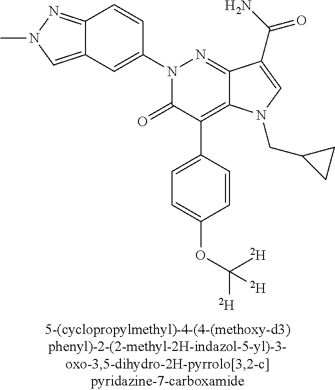 [SMILES: [2H]C([2H])([2H])Oc1ccc(-c2c(=O)n(-c3ccc4nn(C)cc4c3)nc3c(C(N)=O)cn(CC4CC4)c23)cc1] | LC-MS: m/z 472 (M + H)⁺. ¹H NMR (400 MHz, DMSO- d₆) δ: 8.45 (s, 1H), 8.29 (s, 1H), 7.97 (d, J = 1.4 Hz, 1H), 7.66 (d, J = 9.1 Hz, 1H), 7.47- 7.38 (m, 4H), 7.05 (d, J = 8.7 Hz, 3H), 4.20 (s, 3H), 3.41 (d, J = 7.2 Hz, 2H), 0.80-0.69 (m, 1H), 0.35-0.26 (m, 2H), 0.11- 0.06 (m, 2H). |
| 174 | 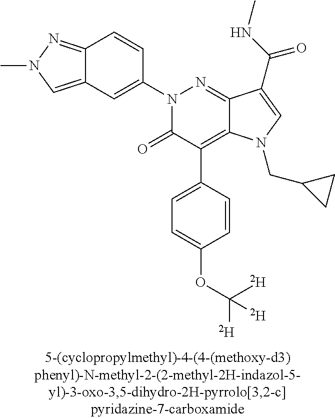 [SMILES: [2H]C([2H])([2H])Oc1ccc(-c2c(=O)n(-c3ccc4nn(C)cc4c3)nc3c(C(=O)NC)cn(CC4CC4)c23)cc1] | LC-MS: m/z 486 (M + H)⁺. ¹H NMR (400 MHz, DMSO- d₆) δ: 8.46 (s, 1H), 8.29 (s, 1H), 7.98 (d, J = 1.4 Hz, 1H), 7.66 (d, J = 9.2 Hz, 1H), 7.52- 7.39 (m, 4H), 7.13-6.98 (m, 2H), 4.21 (s, 3H), 3.41 (d, J = 7.2 Hz, 2H), 2.79 (d, J = 4.8 Hz, 3H), 0.79-0.66 (m, 1H), 0.39-0.23 (m, 2H), 0.11-0.06 (m, 2H). |
| 175 | 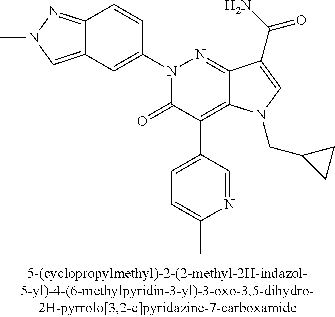 [SMILES: Cc1ccc(-c2c(=O)n(-c3ccc4nn(C)cc4c3)nc3c(C(N)=O)cn(CC4CC4)c23)cn1] | LC-MS: m/z 454 (M + H)⁺. ¹H NMR (400 MHz, DMSO- d₆) δ: 8.55 (d, J = 1.8 Hz, 1H), 8.44 (s, 1H), 8.31 (s, 1H), 7.98 (d, J = 1.3 Hz, 1H), 7.84 (dd, J = 7.9, 2.3 Hz, 1H), 7.66 (d, J = 9.2 Hz, 1H), 7.48 (s, 1H), 7.43 (dd, J = 9.2, 2.0 Hz, 1H), 7.39 (d, J = 8.0 Hz, 1H), 7.01 (s, 1H), 4.20 (s, 3H), 3.39 (d, J = 7.1 Hz, 2H), 2.54 (s, 3H), 0.72-0.59 (m, 1H), 0.35-0.28 (m, 2H), 0.13-0.08 (m, 2H). |
| 176 | 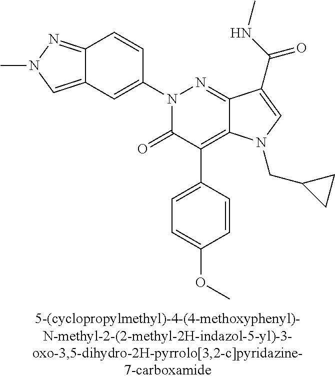 [SMILES: CNC(=O)c1cn(CC2CC2)c2c(-c3ccc(OC)cc3)c(=O)n(-c3ccc4nn(C)cc4c3)nc12] | LC-MS: m/z 483 (M + H)⁺. ¹H NMR (400 MHz, DMSO- d₆) δ: 8.45 (s, 1H), 8.28 (s, 1H), 7.97 (s, 1H), 7.66 (d, J = 9.1 Hz, 1H), 7.44-7.40 (m, 4H), 7.05 (d, J = 8.6 Hz, 2H), 4.21 (s, 3H), 3.82 (s, 3H), 3.42 (d, J = 7.1 Hz, 2H), 2.79 (d, J = 4.7 Hz, 3H), 0.71-0.68 (m, 1H), 0.31-0.26 (m, 2H), 0.10-0.07 (m, 2H). |
| 177 | 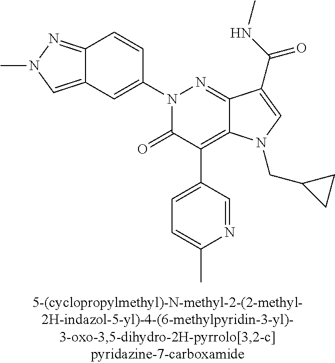 [SMILES: CNC(=O)c1cn(CC2CC2)c2c(-c3ccc(C)nc3)c(=O)n(-c3ccc4nn(C)cc4c3)nc12] | LC-MS: m/z 468 (M + H)⁺. ¹H NMR (400 MHz, DMSO- d₆) δ: 8.56 (d, J = 1.6 Hz, 1H), 8.47 (s, 1H), 8.33 (s, 1H), 7.99 (d, J = 1.2 Hz, 1H), 7.85 (dd, J = 8.0, 2.4 Hz, 1H), 7.67 (d, J = 9.2 Hz, 1H), 7.46-7.38 (m, 3H), 4.21 (s, 3H), 3.40 (d, J = 7.2 Hz, 2H), 2.80 (d, J = 4.8 Hz, 3H), 2.55 (s, 3H), 0.72-0.63 (m, 1H), 0.34-0.28 (m, 2H), 0.12-0.07 (m, 2H) |

### Example 178: Synthesis of 5-(cyclopropylmethyl)-4-(6-cyclopropylpyridin-3-yl)-N,N-dimethyl-2-(2-methyl-2H-indazol-5-yl)-3-oxo-3,5-dihydro-2H-pyrrolo[3,2-c]pyridazine-7-sulfonamide via General Procedure IV

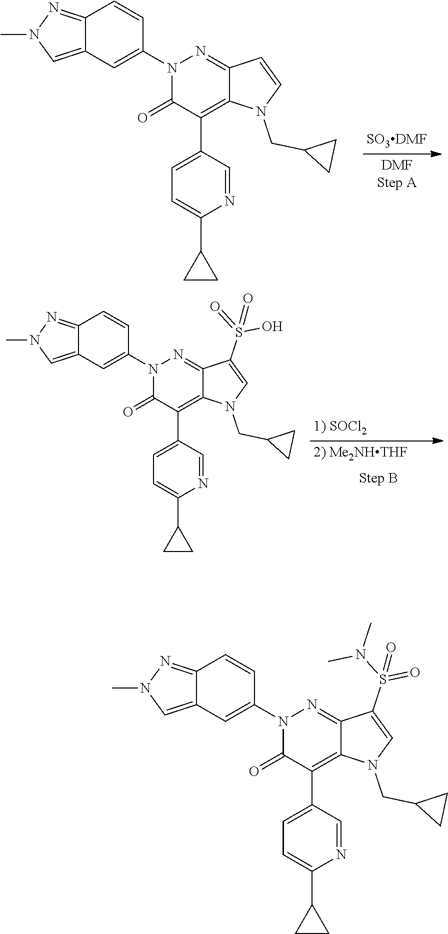

### Step A: 5-(cyclopropylmethyl)-4-(6-cyclopropylpyridin-3-yl)-2-(2-methyl-2H-indazol-5-yl)-3-oxo-3,5-dihydro-2H-pyrrolo[3,2-c]pyridazine-7-sulfonic Acid

To a solution of 5-(cyclopropylmethyl)-4-(6-cyclopropylpyridin-3-yl)-2-(2-methyl-2H-indazol-5-yl)-2,5-dihydro-3H-pyrrolo[3,2-c]pyridazin-3-one (50 mg, 0.115 mmol, 1.0 eq.) in DMF (2 mL) was added DMF.SO₃ (27 mg, 0.173 mmol, 1.5 eq.). The reaction mixture was stirred at 40° C. for 2 hrs. After completion, the reaction mixture was poured into ice water (10 mL) and extracted with EtOAc(10 mL×3). The combined organic layers were washed with brine (10 mL), dried over anhydrous Na₂SO₄, and concentrated under reduced pressure. The resulting residue was purified by flash chromatography to give 5-(cyclopropylmethyl)-4-(6-cyclopropylpyridin-3-yl)-2-(2-methyl-2H-indazol-5-yl)-3-oxo-2H,3H,5H-pyrrolo[3,2-c]pyridazine-7-sulfonic acid (20 mg, 33.7%) as a colorless oil. LC-MS (ESI): m/z 517 [M+H]⁺.

### Step B: 5-(cyclopropylmethyl)-4-(6-cyclopropylpyridin-3-yl)-N,N-dimethyl-2-(2-methyl-2H-indazol-5-yl)-3-oxo-3,5-dihydro-2H-pyrrolo[3,2-c]pyridazine-7-sulfonamide

To a solution of 5-(cyclopropylmethyl)-4-(6-cyclopropylpyridin-3-yl)-2-(2-methyl-2H-indazol-5-yl)-3-oxo-2H,3H,5H-pyrrolo[3,2-c]pyridazine-7-sulfonic acid (100 mg, 0.194 mmol, 1.0 eq.) in DMF (2 mL) was added sulfurous dichloride (0.021 mL, 0.290 mmol, 1.5 eq.), and the reaction mixture was stirred at 70° C. for 1 hr. The reaction was removed allowed to cool to room temperature and dimethylamine (0.140 mL, 0.280 mmol, 3.0 eq., 2M in THF) was added. The reaction mixture was stirred at 25° C. for an additional 2 hrs. After completion, the reaction was mixture was poured into ice water (10 mL) and extracted with EtOAc (10 mL×3). The combined organic layers were washed with brine (10 mL), dried over anhydrous Na₂SO₄ and concentrated under reduced pressure. The resulting residue was purified by flash chromatography and RP-prep-HPLC to give 5-(cyclopropylmethyl)-4-(6-cyclopropylpyridin-3-yl)-N,N-dimethyl-2-(2-methyl-2H-indazol-5-yl)-3-oxo-3,5-dihydro-2H-pyrrolo[3,2-c]pyridazine-7-sulfonamide. ¹H NMR (400 MHz, DMSO-d₆) δ (ppm): 8.51 (d, J=1.7 Hz, 1H), 8.46 (s, 1H), 8.35 (s, 1H), 7.91 (dd, J=1.9, 0.7 Hz, 1H), 7.81 (dd, J=8.0, 2.3 Hz, 1H), 7.67 (d, J=9.2 Hz, 1H), 7.43 (d, J=7.5 Hz, 1H), 7.37 (dd, J=9.2, 2.0 Hz, 1H), 4.20 (s, 3H), 3.43 (d, J=7.1 Hz, 2H), 2.78 (s, 6H), 2.26-2.12 (m, 1H), 1.08-0.91 (m, 4H), 0.69-060 (m, 1H), 0.33-0.29 (m, 2H), 0.19-0.06 (m, 2H). LC-MS (ESI): m/z 544 [M+H]⁺.

The procedure set forth above for General Procedure IV was used to synthesize the following compounds by using appropriate starting materials:

| Example | Structure | Characterization |
|---|---|---|
| 179 | 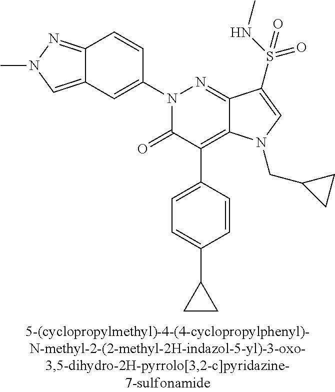 [SMILES: CNS(=O)(=O)c1cn(CC2CC2)c2c(-c3ccc(C4CC4)cc3)c(=O)n(-c3ccc4nn(C)cc4c3)nc12] | LC-MS: m/z 529 (M + H)⁺. ¹H NMR (400 MHz, DMSO- d₆) δ: 8.45 (s, 1H), 8.25 (s, 1H), 7.92 (d, J = 1.4 Hz, 1H), 7.65 (d, J = 9.1 Hz, 1H), 7.43- 7.32 (m, 4H), 7.19 (d, J = 8.2 Hz, 2H), 4.20 (s, 3H), 3.39 (d, J = 7.2 Hz, 2H), 2.56 (d, J = 4.9 Hz, 3H), 2.05-1.95 (m, 1H), 1.06-0.97 (m, 2H), 0.78- 0.65 (m, 3H), 0.33-0.24 (m, 2H), 0.11-0.04 (m, 2H). |
| 180 | 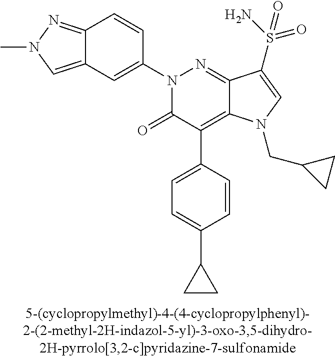 [SMILES: Cn1cc2cc(-n3nc4c(S(N)(=O)=O)cn(CC5CC5)c4c(-c4ccc(C5CC5)cc4)c3=O)ccc2n1] | LC-MS: m/z 515 (M + H)⁺. ¹H NMR (400 MHz, DMSO- d₆) δ: 8.45 (s, 1H), 8.17 (s, 1H), 7.93 (s, 1H), 7.65 (d, J = 9.0 Hz, 1H), 7.43-7.39 (m, 1H), 7.36 (d, J = 8.6 Hz, 4H), 7.18 (d, J = 8.1 Hz, 2H), 4.21 (s, 3H), 3.37 (d, J = 7.1 Hz, 2H), 2.05-1.94 (m, 1H), 1.06- 0.96 (m, 2H), 0.75-0.64 (m, 3H), 0.31-0.27 (m, 2H), 0.09- 0.05 (m, 2H). |
| 181 | 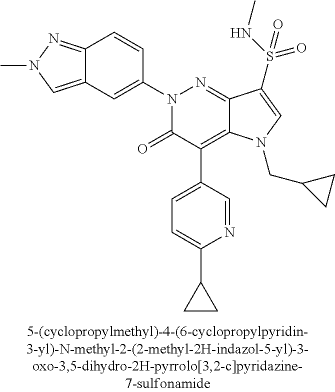 [SMILES: CNS(=O)(=O)c1cn(CC2CC2)c2c(-c3ccc(C4CC4)nc3)c(=O)n(-c3ccc4nn(C)cc4c3)nc12] | LC-MS: m/z 530 (M + H)⁺. ¹H NMR (400 MHz, DMSO- d₆) δ: 8.51 (d, J = 1.8 Hz, 1H), 8.46 (s, 1H), 8.28 (s, 1H), 7.93 (d, J = 1.3 Hz, 1H), 7.81 (dd, J = 8.0, 2.2 Hz, 1H), 7.66 (d, J = 9.2 Hz, 1H), 7.44-7.36 (m, 3H), 4.20 (s, 3H), 3.41 (d, J = 7.1 Hz, 2H), 2.57 (d, J = 4.9 Hz, 3H), 2.24-2.14 (m, 1H), 1.09-0.97 (m, 4H), 0.73- 0.63 (m, 1H), 0.38-0.25 (m, 2H), 0.13-0.08 (m, 2H). |
| 182 | 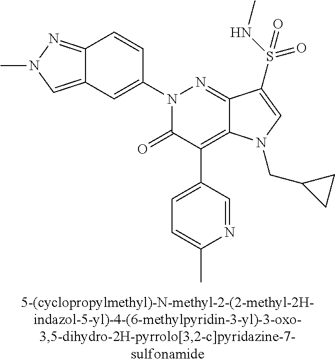 [SMILES: CNS(=O)(=O)c1cn(CC2CC2)c2c(-c3ccc(C)nc3)c(=O)n(-c3ccc4nn(C)cc4c3)nc12] | LC-MS: m/z 504 (M + H)⁺. ¹H NMR (400 MHz, DMSO- d₆) δ: 8.57 (s, 1H), 8.46 (s, 1H), 8.29 (s, 1H), 7.93 (d, J = 1.2 Hz, 1H), 7.86 (d, J = 8.0 Hz, 1H), 7.67 (d, J = 9.2 Hz, 1H), 7.43-7.37 (m, 3H), 4.21 (s, 3H), 3.42 (d, J = 7.2 Hz, 2H), 2.57 (s, 3H), 2.56 (s, 3H), 0.76-0.63 (m, 1H), 0.36- 0.27 (m, 2H), 0.15-0.10 (m, 2H). |
| 183 | 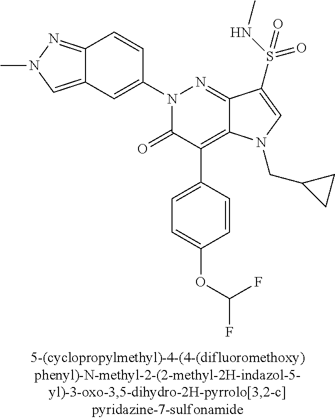 [SMILES: CNS(=O)(=O)c1cn(CC2CC2)c2c(-c3ccc(OC(F)F)cc3)c(=O)n(-c3ccc4nn(C)cc4c3)nc12] | LC-MS: m/z 555 (M + H)⁺. ¹H NMR (400 MHz, DMSO- d₆) δ: 8.47 (s, 1H), 8.28 (s, 1H), 7.93 (d, J = 1.3 Hz, 1H), 7.66 (d, J = 9.2 Hz, 1H), 7.61- 7.56 (m, 2H), 7.40 (dd, J = 9.1, 2.0 Hz, 2H), 7.37 (t, J_{HF} = 74 Hz, 1H), 7.30 (d, J = 8.6 Hz, 2H), 4.20 (s, 3H), 3.39 (d, J = 7.2 Hz, 2H), 2.57 (s, 3H), 0.75-0.63 (m, 1H), 0.34-0.27 (m, 2H), 0.14-0.07 (m, 2H). |
| 184 | 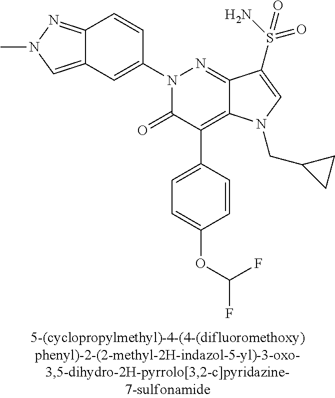 [SMILES: Cn1cc2cc(-n3nc4c(S(N)(=O)=O)cn(CC5CC5)c4c(-c4ccc(OC(F)F)cc4)c3=O)ccc2n1] | LC-MS: m/z 541 (M + H)⁺. ¹H NMR (400 MHz, DMSO- d₆) δ: 8.46 (s, 1H), 8.19 (s, 1H), 7.94 (d, J = 1.3 Hz, 1H), 7.66 (d, J = 9.2 Hz, 1H), 7.60- 7.56 (m, 2H), 7.44-7.38 (m, 3H), 7.37 (t, J_{HF} = 74 Hz, 1H), 7.30 (d, J = 8.6 Hz, 2H), 4.21 (s, 3H), 3.38 (d, J = 7.2 Hz, 2H), 0.74-0.63 (m, 1H), 0.35- 0.27 (m, 2H), 0.14-0.06 (m, 2H). |
| 185 | 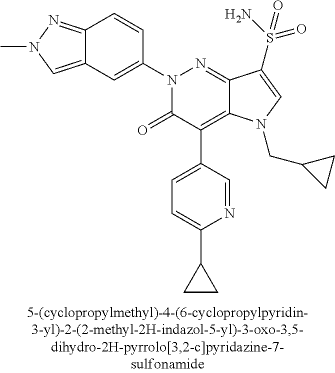 [SMILES: Cn1cc2cc(-n3nc4c(S(N)(=O)=O)cn(CC5CC5)c4c(-c4ccc(C5CC5)nc4)c3=O)ccc2n1] | LC-MS: m/z 516 (M + H)⁺. ¹H NMR (400 MHz, DMSO- d₆) δ: 8.50 (d, J = 1.9 Hz, 1H), 8.46 (s, 1H), 8.20 (s, 1H), 7.94 (d, J = 1.4 Hz, 1H), 7.80 (dd, J = 8.0, 2.3 Hz, 1H), 7.66 (d, J = 9.1 Hz, 1H), 7.46-7.30 (m, 4H), 4.21 (s, 3H), 3.40 (d, J = 7.4 Hz, 2H), 2.19 (s, 1H), 1.05-0.97 (m, 4H), 0.70-0.58 (m, 1H), 0.34-0.30 (m, 2H), 0.12-0.08 (m, 2H). |

### Example 186: Synthesis of 5-(cyclopropylmethyl)-4-[4-(difluoromethoxy)phenyl]-2-(2-methyl-2H-indazol-5-yl)-2H,3H,5H-imidazo[4,5-c]pyridazin-3-one via General Procedure V

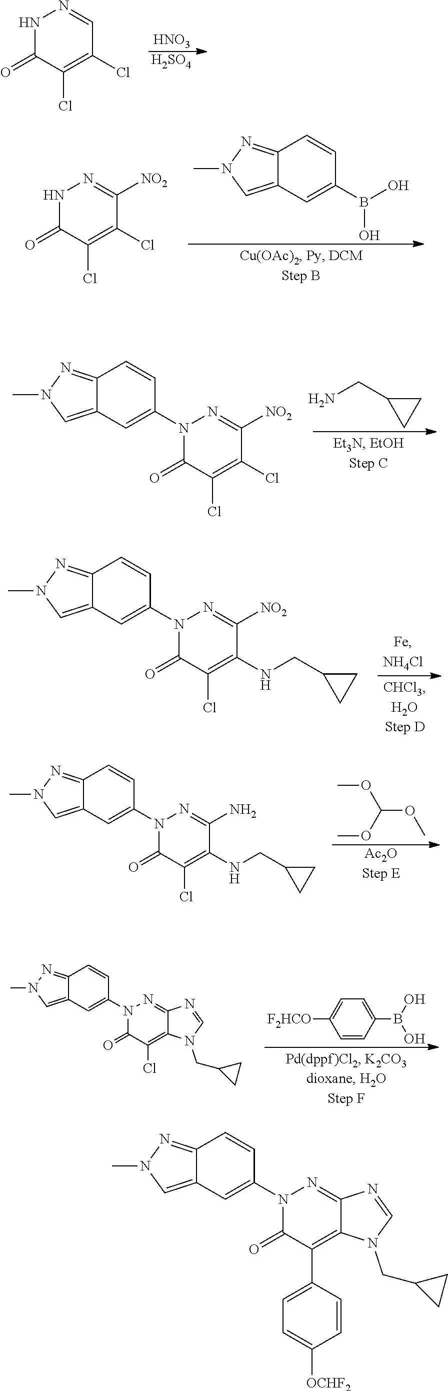

### Step A: 4,5-dichloro-6-nitropyridazin-3(2H)-one

4,5-dichloropyridazin-3(2H)-one (54 g, 327.312 mmol, 1.0 eq.) was added to a stirred solution of concentrated H₂SO₄ (350.0 mL) in portions, the reaction mixture was heated to 100° C., and concentrated HNO₃ (115.8 g, 1145.593 mmol, 3.5 eq.) was added drop-wise at this temperature. The reaction mixture was stirred at 100° C. for 16 hrs. After completion, the reaction mixture was cooled and poured into ice water (2.5 L). The resulting mixture was stirred for one hour, and a precipitation was formed. The mixture was then filtered and the filter cake was dried to give 4,5-dichloro-6-nitro-2,3-dihydropyridazin-3-one (40 g, 58%) as a white solid. LC-MS (ESI): m/z 208[M−H]⁻.

### Step B: 4,5-dichloro-2-(2-methyl-2H-indazol-5-yl)-6-nitropyridazin-3(2H)-one

A mixture of 4,5-dichloro-6-nitro-2,3-dihydropyridazin-3-one (1.2 g, 5.715 mmol, 1.0 eq.), (2-methyl-2H-indazol-5-yl)boronic acid (1.51 g, 8.572 mmol, 1.3 eq.), Cu(OAc)₂ (1.03 g, 5.715 mmol, 1.0 eq.) and pyridine (0.93 mL, 11.430 mmol, 2.0 eq.) in DCM (20 mL) was stirred at 40° C. under N₂ atmosphere for 18 hrs. After completion, the reaction mixture was diluted with water (100 mL) and extracted with DCM (80 mL×3). The combined organic layers were washed with brine (50 mL), dried over anhydrous Na₂SO₄ and concentrated under reduced pressure. The resulting residue was purified by column chromatography on silica gel to give 4,5-dichloro-2-(2-methyl-2H-indazol-5-yl)-6-nitro-2,3-dihydropyridazin-3-one (1.44 g, 74%) as a yellow solid. LC-MS (ESI): m/z 340 [M+H]⁺

### Step C:4-chloro-5-((cyclopropylmethyl)amino)-2-(2-methyl-2H-indazol-5-yl)-6-nitropyridazin-3(2H)-one

A solution of 4,5-dichloro-2-(2-methyl-2H-indazol-5-yl)-6-nitro-2,3-dihydropyridazin-3-one (800 mg, 2.352 mmol, 1.0 eq.), cyclopropylmethanamine (0.25 mL, 2.822 mmol, 1.2 eq.) and Et₃N (0.5 mL, 3.528 mmol, 1.5 eq.) in EtOH (10 mL) was stirred at 40° C. for 18 hrs. The reaction mixture was concentrated under reduced pressure, the residue was diluted with water (30 mL) and extracted with DCM (50 mL×2). The combined organic layers were washed with brine (30 mL), dried over anhydrous Na₂SO₄ and concentrated under reduced pressure. The resulting residue was purified by column chromatography on silica gel to give 4-chloro-5-[(cyclopropylmethyl)amino]-2-(2-methyl-2H-indazol-5-yl)-6-nitro-2,3-dihydropyridazin-3-one (640 mg, 73%) as a brown solid. LC-MS (ESI): m/z 375 [M+H]⁺

### Step D: 6-amino-4-chloro-5-((cyclopropylmethyl)amino)-2-(2-methyl-2H-indazol-5-yl)pyridazin-3(2H)-one

To a solution of 4-chloro-5-[(cyclopropylmethyl)amino]-2-(2-methyl-2H-indazol-5-yl)-6-nitro-2,3-dihydropyridazin-3-one (200 mg, 0.534 mmol, 1.0 eq.) in CHCl₃ (4 mL) and H₂O (4 mL) was added Fe powder (90 mg, 1.601 mmol, 3.0 eq.) and NH₄Cl (143 mg, 2.668 mmol, 5.0 eq.). The reaction mixture was stirred at 50° C. for 18 hrs. After completion, the reaction mixture was filtered through a short pad of Celite®. The filtrate was concentrated under reduced pressure and the residue was purified column chromatography on silica gel to give 6-amino-4-chloro-5-[(cyclopropylmethyl)amino]-2-(2-methyl-2H-indazol-5-yl)-2,3-dihydropyridazin-3-one (150 mg, 82%) as a yellow solid. LC-MS (ESI): m/z 345 [M+H]⁺

### Step E:4-chloro-5-(cyclopropylmethyl)-2-(2-methyl-2H-indazol-5-yl)-2,5-dihydro-3H-imidazo[4,5-c]pyridazin-3-one

A solution of 6-amino-4-chloro-5-[(cyclopropylmethyl)amino]-2-(2-methyl-2H-indazol-5-yl)-2,3-dihydropyridazin-3-one (200 mg, 0.580 mmol, 1.0 eq.) in trimethyl orthoformate (2 mL) and Ac₂O (1 mL) was stirred at 90° C. for 1 hr. The reaction mixture was concentrated under reduced pressure and the residue was purified by column chromatography on silica gel to give 4-chloro-5-(cyclopropylmethyl)-2-(2-methyl-2H-indazol-5-yl)-2H,3H,5H-imidazo[4,5-c]pyridazin-3-one (200 mg, 97%) as a yellow solid. LC-MS (ESI): m/z 355 [M+H]⁺

### Step F: 5-(cyclopropylmethyl)-4-(4-(difluoromethoxy)phenyl)-2-(2-methyl-2H-indazol-5-yl)-2,5-dihydro-3H-imidazo[4,5-c]pyridazin-3-one

To a solution of 4-chloro-5-(cyclopropylmethyl)-2-(2-methyl-2H-indazol-5-yl)-2H,3H,5H-imidazo[4,5-c]pyridazin-3-one (70 mg, 0.197 mmol, 1.0 eq.), (4-(difluoromethoxy)phenyl)boronic acid (56 mg, 0.296 mmol, 1.5 eq.) and Pd(dppf)Cl₂ (8 mg, 0.010 mmol, 0.05 eq.) in dioxane (1.6 mL) was added a solution of K₂CO₃ (55 mg, 0.395 mmol, 2.0 eq.) in water (0.4 mL). The reaction mixture was stirred at 100° C. under N₂ atmosphere for 18 hrs. After completion, the reaction mixture was filtered through a short pad of Celite®, the filtrate was concentrated under reduced pressure, and the residue was purified by column chromatography on silica gel and Prep-TLC to give 5-(cyclopropylmethyl)-4-[4-(difluoromethoxy)phenyl]-2-(2-methyl-2H-indazol-5-yl)-2H,3H,5H-imidazo[4,5-c]pyridazin-3-one. ¹H NMR (400 MHz, DMSO-d₆) δ (ppm): 8.60 (s, 1H), 8.45 (s, 1H), 7.91 (d, J=1.4 Hz, 1H), 7.66 (d, J=9.2 Hz, 1H), 7.60 (d, J=8.7 Hz, 2H), 7.38 (dd, J=9.2, 2.0 Hz, 1H), 7.35 (t, J_{HF}=74 Hz, 1H), 7.30 (d, J=8.6 Hz, 2H), 4.20 (s, 3H), 3.49 (d, J=7.2 Hz, 2H), 0.64-0.57 (m, 1H), 0.36-0.24 (m, 2H), 0.13-0.02 (m, 2H). LC-MS (ESI): m/z 463 [M+H]⁺

The procedure set forth above for General Procedure V was used to synthesize the following compounds by using appropriate starting materials:

| Example | Structure | Characterization |
|---|---|---|
| 187 | 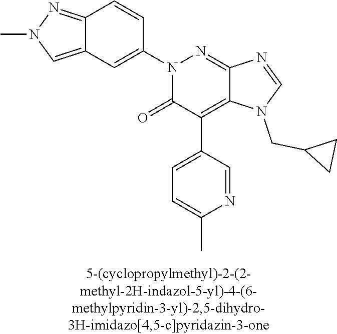 [SMILES: Cc1ccc(-c2c(=O)n(-c3ccc4nn(C)cc4c3)nc3ncn(CC4CC4)c23)cn1] | LC-MS: m/z 412 (M + H)⁺. ¹H NMR (400 MHz, DMSO- d₆) δ: 8.62 (s, 1H), 8.59 (s, 1H), 8.45 (s, 1H), 7.92 (s, 1H), 7.87 (d, J = 7.8 Hz, 1H), 7.66 (d, J = 9.1 Hz, 1H), 7.44- 7.35 (m, 2H), 4.21 (s, 3H), 3.51 (d, J = 7.2 Hz, 2H), 2.54 (s, 3H), 0.65-0.54 (m, 1H), 0.37-0.28 (m, 2H), 0.18-0.09 (m, 2H). |
| 188 | 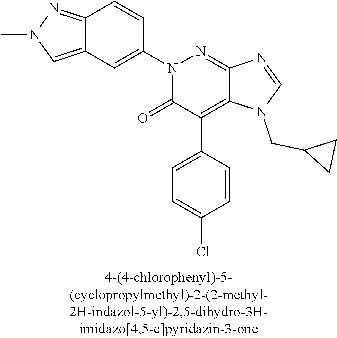 [SMILES: Cn1cc2cc(-n3nc4ncn(CC5CC5)c4c(-c4ccc(Cl)cc4)c3=O)ccc2n1] | LC-MS: m/z 431 (M + H)⁺. ¹H NMR (400 MHz, DMSO- d₆) δ: 8.61 (s, 1H), 8.45 (s, 1H), 7.92 (d, J = 1.4 Hz, 1H), 7.66 (d, J = 9.1 Hz, 1H), 7.58- 7.56 (m, 4H), 7.39 (dd, J = 9.1, 1.9 Hz, 1H), 4.21 (s, 3H), 3.49 (d, J = 7.2 Hz, 2H), 0.66- 0.51 (m, 1H), 0.36-0.30 (m, 2H), 0.15-0.10 (m, 2H). |
| 189 | 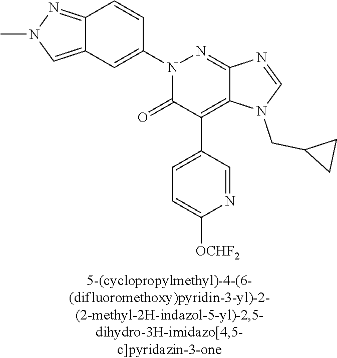 [SMILES: Cn1cc2cc(-n3nc4ncn(CC5CC5)c4c(-c4ccc(OC(F)F)nc4)c3=O)ccc2n1] | LC-MS: m/z 464 (M + H)⁺. ¹H NMR (400 MHz, DMSO- d₆) δ: 8.65 (s, 1H), 8.45 (s, 2H), 8.14 (dd, J = 8.5, 2.3 Hz, 1H), 7.93 (d, J = 1.4 Hz, 1H), 7.81 (t, J_{HF} = 72.8 Hz, 1H), 7.67 (d, J = 9.2 Hz, 1H), 7.39 (dd, J = 9.2, 2.0 Hz, 1H), 7.25 (d, J = 8.5 Hz, 1H), 4.21 (s, 3H), 3.52 (d, J = 7.2 Hz, 2H), 0.68-0.62 (m, 1H), 0.38-0.29 (m, 2H), 0.20-0.14 (m, 2H). |
| 190 | 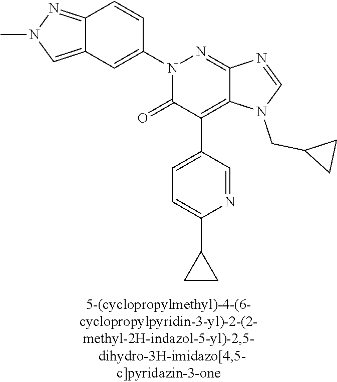 [SMILES: Cn1cc2cc(-n3nc4ncn(CC5CC5)c4c(-c4ccc(C5CC5)nc4)c3=O)ccc2n1] | LC-MS: m/z 438 (M + H)⁺. ¹H NMR (400 MHz, DMSO- d₆) δ: 8.61 (s, 1H), 8.53 (d, J = 1.7 Hz, 1H), 8.45 (s, 1H), 7.92 (dd, J = 1.9, 0.6 Hz, 1H), 7.82 (dd, J = 8.0, 2.2 Hz, 1H), 7.66 (d, J = 9.2 Hz, 1H), 7.45-7.41 (m, 1H), 7.38 (dd, J = 9.2, 2.0 Hz, 1H), 4.21 (s, 3H), 3.50 (d, J = 7.2 Hz, 2H), 2.25-2.12 (m, 1H), 1.04-0.92 (m, 4H), 0.63- 0.59 (m, 1H), 0.40-0.25 (m, 2H), 0.18-0.06 (m, 2H). |
| 191 | 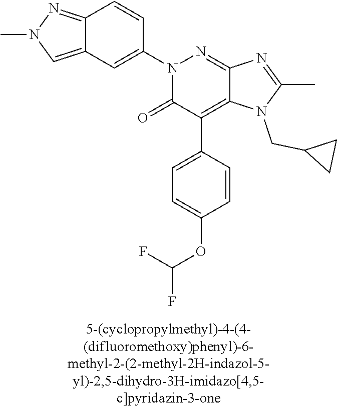 [SMILES: Cc1nc2nn(-c3ccc4nn(C)cc4c3)c(=O)c(-c3ccc(OC(F)F)cc3)c2n1CC1CC1] | LC-MS: m/z 477 (M + H)⁺. ¹H NMR (400 MHz, DMSO- d₆) δ: 8.44 (s, 1H), 7.89 (d, J = 1.8 Hz, 1H), 7.65 (d, J = 9.2 Hz, 1H), 7.56 (d, J = 8.6 Hz, 2H), 7.37 (dd, J = 9.2, 2.0 Hz, 1H), 7.35 (t, J_{HF} = 74.0 Hz, 1H), 7.30 (d, J = 8.5 Hz, 2H), 4.20 (s, 3H), 3.55 (d, J = 6.8 Hz, 2H), 2.53 (s, 3H), 0.65- 0.60 (m, 1H), 0.29-0.22 (m, 2H), −0.03-−0.10 (m, 2H). |
| 192 | 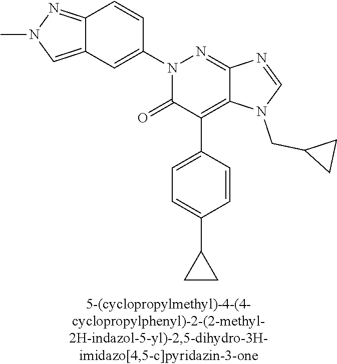 [SMILES: Cn1cc2cc(-n3nc4ncn(CC5CC5)c4c(-c4ccc(C5CC5)cc4)c3=O)ccc2n1] | LC-MS: m/z 437 (M + H)⁺. ¹H NMR (400 MHz, DMSO- d₆) δ: 8.58 (s, 1H), 8.44 (s, 1H), 7.90 (s, 1H), 7.65 (d, J = 9.1 Hz, 1H), 7.40-7.36 (m, 3H), 7.19 (d, J = 8.1 Hz, 2H), 4.21 (s, 3H), 3.49 (d, J = 7.2 Hz, 2H), 2.02-1.95 (m, 1H), 1.05-1.00 (m, 2H), 0.75-0.73 (m, 2H), 0.65-0.59 (m, 1H), 0.32-0.28 (m, 2H), 0.10-0.06 (m, 2H). |
| 185 | 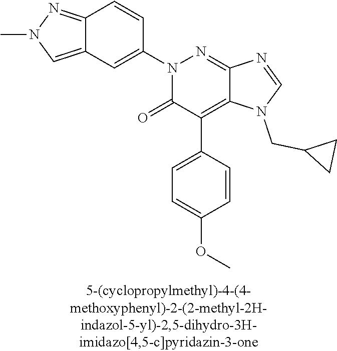 [SMILES: COc1ccc(-c2c(=O)n(-c3ccc4nn(C)cc4c3)nc3ncn(CC4CC4)c23)cc1] | LC-MS: m/z 427 (M + H)⁺. ¹H NMR (400 MHz, DMSO- d₆) δ: 8.58 (s, 1H), 8.44 (s, 1H), 7.91 (d, J = 1.4 Hz, 1H), 7.65 (d, J = 9.1 Hz, 1H), 7.46 (d, J = 8.6 Hz, 2H), 7.38 (dd, J = 9.1, 1.9 Hz, 1H), 7.05 (d, J = 8.7 Hz, 2H), 4.21 (s, 3H), 3.82 (s, 3H), 3.52 (d, J = 7.2 Hz, 2H), 0.65-0.59 (m, 1H), 0.34-0.29 (m, 2H), 0.12-0.07 (m, 2H). |
| 194 | 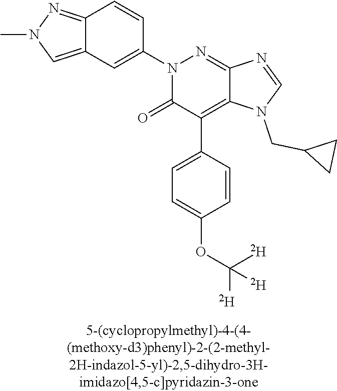 [SMILES: [2H]C([2H])([2H])Oc1ccc(-c2c(=O)n(-c3ccc4nn(C)cc4c3)nc3ncn(CC4CC4)c23)cc1] | LC-MS: m/z 430 (M + H)⁺. ¹H NMR (400 MHz, DMSO- d₆) δ: 8.60 (s, 1H), 8.45 (s, 1H), 7.91 (d, J = 1.4 Hz, 1H), 7.65 (d, J = 9.1 Hz, 1H), 7.46 (d, J = 8.7 Hz, 2H), 7.38 (dd, J = 9.1, 2.0 Hz, 1H), 7.05 (d, J = 8.7 Hz, 2H), 4.21 (s, 3H), 3.53 (d, J = 7.2 Hz, 2H), 0.67- 0.56 (m, 1H), 0.32-0.24 (m, 2H), 0.12-0.08 (m, 2H). |
| 195 | 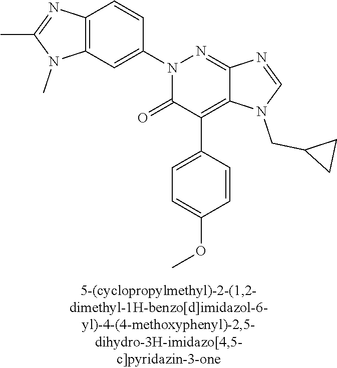 [SMILES: COc1ccc(-c2c(=O)n(-c3ccc4nc(C)n(C)c4c3)nc3ncn(CC4CC4)c23)cc1] | LC-MS: m/z 441 (M + H)⁺. ¹H NMR (400 MHz, DMSO- d₆) δ: 8.58 (s, 1H), 7.72 (d, J = 2.0 Hz, 1H), 7.58 (d, J = 8.4 Hz, 1H), 7.51-7.42 (m, 2H), 7.31 (dd, J = 8.4, 2.0 Hz, 1H), 7.06 (d, J = 8.8 Hz, 2H), 3.83 (s, 3H), 3.75 (s, 3H), 3.53 (d, J = 7.2 Hz, 2H), 2.56 (s, 3H), 0.66-0.53 (m, 1H), 0.38-0.26 (m, 2H), 0.18-0.07 (m, 2H). |

### Example 196: Synthesis of 5-(cyclopropylmethyl)-4-[4-(difluoromethoxy)phenyl]-7-methyl-2-(2-methyl-2H-indazol-5-yl)-2H,3H,5H,6H,7H-imidazolidino[4,5-c]pyridazine-3,6-dione via General Procedure VI (Method A)

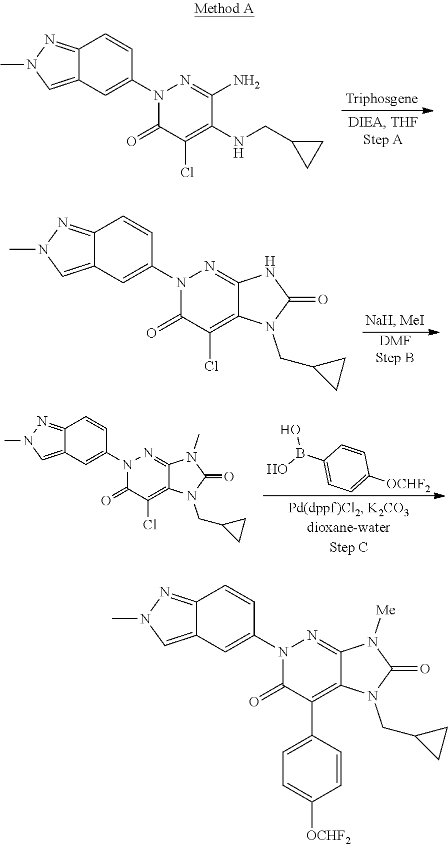 [SMILES: Cn1cc2cc(-n3nc(N)c(NCC4CC4)c(Cl)c3=O)ccc2n1 ; Cn1cc2cc(-n3nc4[nH]c(=O)n(CC5CC5)c4c(Cl)c3=O)ccc2n1 ; "DMFStep B" ; Cn1cc2cc(-n3nc4c(c(Cl)c3=O)n(CC3CC3)c(=O)n4C)ccc2n1 ; OB(O)c1ccc(OC(F)F)cc1 ; Cn1cc2cc(-n3nc4c(c(-c5ccc(OC(F)F)cc5)c3=O)n(CC3CC3)c(=O)n4C)ccc2n1]

### Step A: 4-chloro-5-(cyclopropylmethyl)-2-(2-methyl-2H-indazol-5-yl)-2,7-dihydro-3H-imidazo[4,5-c]pyridazine-3,6(5H)-dione

To a solution of 6-amino-4-chloro-5-((cyclopropylmethyl)amino)-2-(2-methyl-2H-indazol-5-yl)pyridazin-3(2H)-one (100 mg, 0.29 mmol, 1.0 eq.) and DIPEA (0.24 mL, 1.45 mmol, 5.0 eq.) in THE (3 mL) was added a solution of triphosgene (52 mg, 0.174 mmol, 0.6 eq.) in THE (1 mL) drop-wise at 0° C. The reaction mixture was stirred at room temperature for 1.5 hrs. After completion, the reaction was quenched with NaHCO₃(sat. aq.) (10 mL) at 0° C., the aqueous layer was extracted with EtOAc (50 mL×3), and the combined organic layers were washed with brine (30 mL), dried over anhydrous Na₂SO₄ and concentrated under reduced pressure. The resulting residue was purified by column chromatography on silica gel to give 4-chloro-5-(cyclopropylmethyl)-2-(2-methyl-2H-indazol-5-yl)-2,7-dihydro-3H-imidazo[4,5-c]pyridazine-3,6(5H)-dione (80 mg, 74%) as a yellow solid. LC-MS (ESI): m/z 371 [M+H]⁺

### Step B: 4-chloro-5-(cyclopropylmethyl)-7-methyl-2-(2-methyl-2H-indazol-5-yl)-2,7-dihydro-3H-imidazo[4,5-c]pyridazine-3,6(5H)-dione

To a solution of 4-chloro-5-(cyclopropylmethyl)-2-(2-methyl-2H-indazol-5-yl)-2,7-dihydro-3H-imidazo[4,5-c]pyridazine-3,6(5H)-dione (40 mg, 0.108 mmol, 1.0 eq.) in DMF (3 mL) was added NaH (8 mg, 0.324 mmol, 3.0 eq.) at 0° C. The reaction mixture was stirred at 0° C. for 5 min, then Mel (46 mg, 0.324 mmol, 3.0 eq.) was added and the resulting mixture was stirred at room temperature for 2 hrs. After completion, the reaction was quenched with NH₄Cl (sat. aq.) (15 mL) at 0° C., the aqueous layer was extracted with EtOAc (40 mL×3), and the combined organic layers were washed with brine (30 mL), dried over anhydrous Na₂SO₄ and concentrated under reduced pressure. The resulting residue was purified by column chromatography on silica gel to give 4-chloro-5-(cyclopropylmethyl)-7-methyl-2-(2-methyl-2H-indazol-5-yl)-2,7-dihydro-3H-imidazo[4,5-c]pyridazine-3, 6(5H)-dione (40 mg, 88%) as a white solid. LC-MS (ESI): m/z 385 [M+H]⁺

### Step C: 5-(cyclopropylmethyl)-4-(4-(difluoromethoxy)phenyl)-7-methyl-2-(2-methyl-2H-indazol-5-yl)-2,7-dihydro-3H-imidazo[4,5-c]pyridazine-3,6(5H)-dione

To a solution of 4-chloro-5-(cyclopropylmethyl)-7-methyl-2-(2-methyl-2H-indazol-5-yl)-2,7-dihydro-3H-imidazo[4,5-c]pyridazine-3,6(5H)-dione (25 mg, 0.065 mmol, 1.0 eq.), (4-(difluoromethoxy)phenyl)boronic acid (15 mg, 0.078 mmol, 1.2 eq.) and Pd(dppf)Cl₂ (10 mg, 0.013 mmol, 0.2 eq.) in dioxane (0.8 mL) was added a solution of K₂CO₃ (27 mg, 0.195 mmol, 3.0 eq.) in H₂O (0.2 mL). The reaction mixture was stirred at 100° C. under atmosphere N₂ for 2 hrs. The reaction mixture was diluted with water (15 mL), extracted with EtOAc (20 mL×3), and the combined organic layers were washed with brine (10 mL), dried over anhydrous Na₂SO₄ and concentrated under reduced pressure. The resulting residue was purified by RP-prep-HPLC to afford 5-(cyclopropylmethyl)-4-[4-(difluoromethoxy)phenyl]-7-methyl-2-(2-methyl-2H-indazol-5-yl)-2H,3H,5H,6H,7H-imidazolidino[4,5-c]pyridazine-3,6-dione. ¹H NMR (400 MHz, DMSO-d₆) δ (ppm): 8.43 (s, 1H), 7.88 (d, J=1.2 Hz, 1H), 7.65 (d, J=9.2 Hz, 1H), 7.54 (d, J=8.8 Hz, 2H), 7.38 (d, J=8.4 Hz, 1H), 7.36 (t, J_{HF}=74 Hz, 1H), 7.30 (d, J=8.8 Hz, 2H), 4.20 (s, 3H), 3.29 (d, J=7.2 Hz, 2H), 3.24 (s, 3H), 0.46-0.40 (m, 1H), 0.24-0.19 (m, 2H), −0.02-0.01 (m, 2H). LC-MS (ESI): m/z 493 [M+H]⁺.

### Example 197: Synthesis of 5-(cyclopropylmethyl)-4-(4-(difluoromethoxy)phenyl)-2-(2-methyl-2H-indazol-5-yl)-2,7-dihydro-3H-imidazo[4,5-c]pyridazine-3,6(5H)-dione via General Procedure VI (Method B)

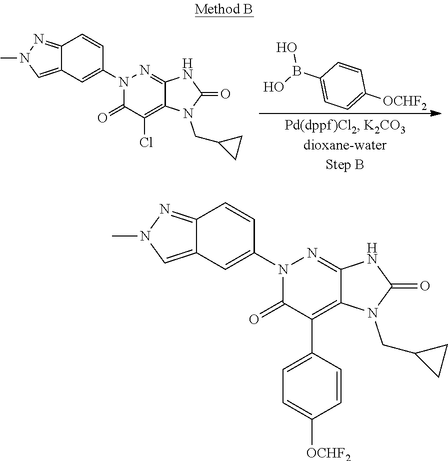 [SMILES: Cn1cc2cc(-n3nc4[nH]c(=O)n(CC5CC5)c4c(Cl)c3=O)ccc2n1 ; OB(O)c1ccc(OC(F)F)cc1 ; Cn1cc2cc(-n3nc4[nH]c(=O)n(CC5CC5)c4c(-c4ccc(OC(F)F)cc4)c3=O)ccc2n1]

### Step B: 5-(cyclopropylmethyl)-4-(4-(difluoromethoxy)phenyl)-2-(2-methyl-2H-indazol-5-yl)-2,7-dihydro-3H-imidazo[4,5-c]pyridazine-3,6(5H)-dione

To a solution of 4-chloro-5-(cyclopropylmethyl)-2-(2-methyl-2H-indazol-5-yl)-2,7-dihydro-3H-imidazo[4,5-c]pyridazine-3,6(5H)-dione (60 mg, 0.162 mmol, 1.0 eq.) (4-(difluoromethoxy)phenyl)boronic acid (36 mg, 0.194 mmol, 1.2 eq.) and Pd(dppf)Cl₂ (23 mg, 0.032 mmol, 0.2 eq.) in dioxane (2.5 mL) was added a solution of K₂CO₃ (67 mg, 0.485 mmol, 3.0 eq.) in H₂O (0.5 mL). The reaction mixture was degassed with N₂, sealed in a tube and heated at 100° C. under microwave irradiation for 2 hrs. After completion, the reaction mixture was filtered through a short pad of Celite®, the filtrate was concentrated under reduced pressure, and the residue was purified by column chromatography on silica gel and Rp-prep-HPLC to afford 5-(cyclopropylmethyl)-4-(4-(difluoromethoxy)phenyl)-2-(2-methyl-2H-indazol-5-yl)-2,7-dihydro-3H-imidazo[4,5-c]pyridazine-3,6(5H)-dione (Example 197).

¹H NMR (400 MHz, DMSO-d₆) δ (ppm): 8.40 (s, 1H), 7.84 (s, 1H), 7.62 (d, J=9.2 Hz, 1H), 7.54 (d, J=8.5 Hz, 2H), 7.38-7.31 (m, 1H), 7.33 (t, J_{HF}=74 Hz, 1H), 7.28 (d, J=8.5 Hz, 2H), 4.18 (s, 3H), 3.24 (d, J=7.0 Hz, 2H), 0.50-0.32 (m, 1H), 0.23-0.18 (m, 2H), 0.18-0.05 (m, 2H). LC-MS (ESI): m/z 479 [M+H]⁺.

The procedure set forth above for General Procedure VI (Method A) was used to synthesize the following compounds by using appropriate starting materials:

| Example | Structure | Characterization |
|---|---|---|
| 198 | 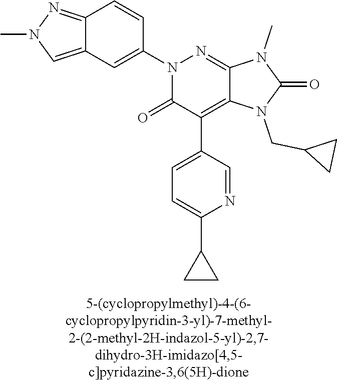 [SMILES: Cn1cc2cc(-n3nc4c(c(-c5ccc(C6CC6)nc5)c3=O)n(CC3CC3)c(=O)n4C)ccc2n1] | LC-MS: m/z 468 (M + H)⁺. ¹H NMR (400 MHz, DMSO- d₆) δ: 8.47 (d, J = 2.0 Hz, 1H), 8.43 (s, 1H), 7.88 (d, J = 1.6 Hz, 1H), 7.76 (dd, J = 8.0, 2.0 Hz, 1H), 7.64 (d, J = 9.2 Hz, 1H), 7.42 (d, J = 8.0 Hz, 1H), 7.37 (dd, J = 9.2, 2.0 Hz, 1H), 4.20 (s, 3H), 3.30 (d, J = 7.2 Hz, 2H), 3.24 (s, 3H), 2.20- 2.15 (m, 1H), 1.01-0.97 (m, 4H), 0.47-0.36 (m, 1H), 0.26- 0.20 (m, 2H), 0.03-0.01 (m, 2H). |
| 199 | 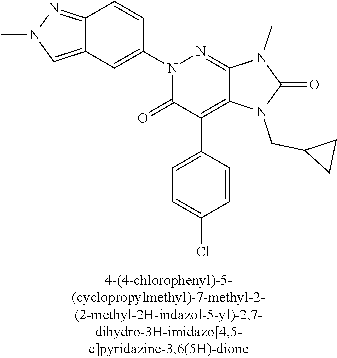 [SMILES: Cn1cc2cc(-n3nc4c(c(-c5ccc(Cl)cc5)c3=O)n(CC3CC3)c(=O)n4C)ccc2n1] | LC-MS: m/z 461 (M + H)⁺. ¹H NMR (400 MHz, DMSO- d₆) δ: 8.42 (s, 1H), 7.88 (d, J = 1.3 Hz, 1H), 7.64 (d, J = 9.1 Hz, 1H), 7.57 (d, J = 8.6 Hz, 2H), 7.51 (d, J = 8.6 Hz, 2H), 7.37 (dd, J = 9.2, 2.0 Hz, 1H), 4.20 (s, 3H), 3.27 (d, J = 7.2 Hz, 2H), 3.24 (s, 3H), 0.48- 0.39 (m, 1H), 0.24-0.20 (m, 2H), 0.04-0.00 (m, 2H). |
| 200 | 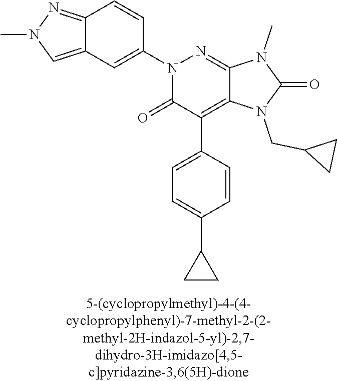 [SMILES: Cn1cc2cc(-n3nc4c(c(-c5ccc(C6CC6)cc5)c3=O)n(CC3CC3)c(=O)n4C)ccc2n1] | LC-MS: m/z 467 (M + H)⁺. ¹H NMR (400 MHz, DMSO- d₆) δ: 8.42 (s, 1H), 7.90-7.84 (m, 1H), 7.63 (d, J = 9.2 Hz, 1H), 7.36 (dd, J = 9.2, 2.0 Hz, 1H), 7.32 (d, J = 8.2 Hz, 2H), 7.18 (d, J = 8.2 Hz, 2H), 4.19 (s, 3H), 3.28 (d, J = 7.2 Hz, 2H), 3.23 (s, 3H), 2.04-1.94 (m, 1H), 1.03-0.91 (m, 2H), 0.78-0.67 (m, 2H), 0.50-0.37 (m, 1H), 0.26-0.14 (m, 2H), - 0.04-0.0 (m, 2H). |
| 201 | 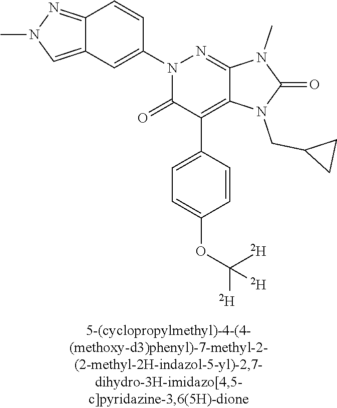 [SMILES: [2H]C([2H])([2H])Oc1ccc(-c2c(=O)n(-c3ccc4nn(C)cc4c3)nc3c2n(CC2CC2)c(=O)n3C)cc1] | LC-MS: m/z 460 (M + H)⁺. ¹H NMR (400 MHz, DMSO- d₆) δ: 8.42 (s, 1H), 7.90-7.84 (m, 1H), 7.64 (d, J = 9.2 Hz, 1H), 7.42-7.34 (m, 3H), 7.08- 6.96 (m, 2H), 4.20 (s, 3H), 3.30 (d, J = 7.2 Hz, 2H), 3.24 (s, 3H), 0.52-0.42 (m, 1H), 0.25-0.17 (m, 2H), −0.00- −0.04 (m, 2H). |
| 202 | 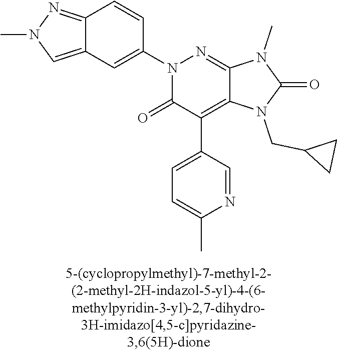 [SMILES: Cc1ccc(-c2c(=O)n(-c3ccc4nn(C)cc4c3)nc3c2n(CC2CC2)c(=O)n3C)cn1] | LC-MS: m/z 442 (M + H)⁺. ¹H NMR (400 MHz, DMSO- d₆) δ: 8.52 (s, 1H), 8.43 (s, 1H), 7.88 (s, 1H), 7.80 (d, J = 7.9 Hz, 1H), 7.65 (d, J = 9.0 Hz, 1H), 7.38 (t, J = 8.6 Hz, 2H), 4.19 (s, 3H), 3.29 (s, 2H), 3.25 (s, 3H), 2.54 (s, 3H), 0.51-0.39 (m, 1H), 0.24- 0.19 (m, 2H), 0.02-0.00 (m, 2H). |

The procedure set forth above for General Procedure VI (Method B) was used to synthesize the following compounds by using appropriate starting materials:

| Example | Structure | Characterization |
|---|---|---|
| 203 | 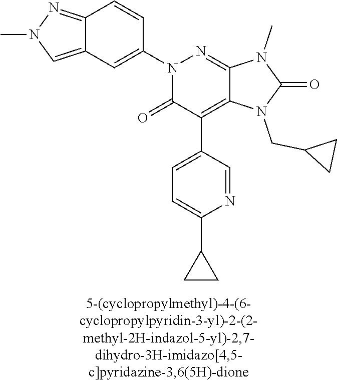 | LC-MS: m/z 454 (M + H)⁺. ¹H NMR (400 MHz, DMSO- d₆) δ: 11.89 (s, 1H), 8.48 (d, J = 1.6 Hz, 1H), 8.41 (s, 1H), 7.85 (d, J = 1.3 Hz, 1H), 7.76 (dd, J = 8.0, 2.0 Hz, 1H), 7.63 (d, J = 9.1 Hz, 1H), 7.41 (d, J = 8.0 Hz, 1H), 7.34 (dd, J = 9.2, 1.7 Hz, 1H), 4.19 (s, 3H), 3.25 (d, J = 6.9 Hz, 2H), 2.23- 2.13 (m, 1H), 1.06-0.90 (m, 4H), 0.45-0.35 (m, 1H), 0.25- 0.20 (m, 2H), −0.10-0.0 (m, 2H). |
| 204 | 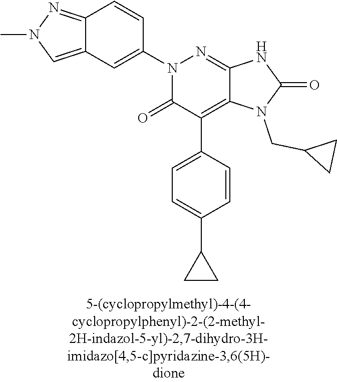 [SMILES: Cn1cc2cc(-n3nc4[nH]c(=O)n(CC5CC5)c4c(-c4ccc(C5CC5)cc4)c3=O)ccc2n1] | LC-MS: m/z 453 (M + H)⁺. ¹H NMR (400 MHz, DMSO- d₆) δ: 11.84 (s, 1H), 8.39 (s, 1H), 7.82 (d, J = 1.4 Hz, 1H), 7.61 (d, J = 9.2 Hz, 1H), 7.36- 7.27 (m, 3H), 7.16 (d, J = 8.1 Hz, 2H), 4.18 (s, 3H), 3.22 (d, J = 7.1 Hz, 2H), 2.03-1.93 (m, 1H), 1.05-0.94 (m, 2H), 0.75- 0.66 (m, 2H), 0.48-0.37 (m, 1H), 0.20-0.15 (m, 2H), −0.06- 0.0 (m, 2H). |
| 205 | 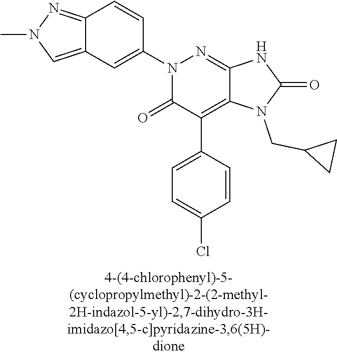 [SMILES: Cn1cc2cc(-n3nc4[nH]c(=O)n(CC5CC5)c4c(-c4ccc(Cl)cc4)c3=O)ccc2n1] | LC-MS: m/z 447 (M + H)⁺. ¹H NMR (400 MHz, DMSO- d₆) δ: 11.92 (s, 1H), 8.41 (s, 1H), 7.84 (dd, J = 2.4, 0.8 Hz, 1H), 7.62 (d, J = 9.2 Hz, 1H), 7.58-7.48 (m, 4H), 7.34 (dd, J = 9.2, 2.0 Hz, 1H), 4.19 (s, 3H), 3.24 (d, J = 7.0 Hz, 2H), 0.46-0.36 (m, 1H), 0.27-0.18 (m, 2H), 0.02-−0.01 (m, 2H). |
| 206 | 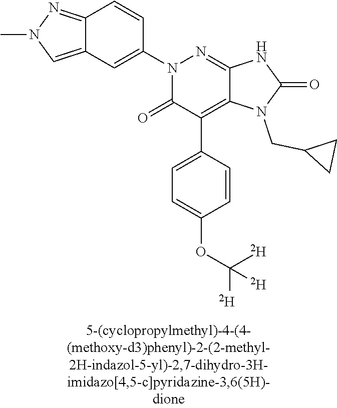 [SMILES: [2H]C([2H])([2H])Oc1ccc(-c2c(=O)n(-c3ccc4nn(C)cc4c3)nc3[nH]c(=O)n(CC4CC4)c23)cc1] | LC-MS: m/z 446 (M + H)⁺. ¹H NMR (400 MHz, DMSO- d₆) δ: 11.80 (s, 1H), 8.40 (s, 1H), 7.84 (d, J = 1.4 Hz, 1H), 7.62 (d, J = 9.2 Hz, 1H), 7.39 (d, J = 8.0 Hz, 2H), 7.34 (dd, J = 9.2, 2.0 Hz, 1H), 7.03 (d, J = 8.0 Hz, 2H), 4.19 (s, 3H), 3.27 (d, J = 7.1 Hz, 2H), 0.50- 0.40 (m, 1H), 0.26-0.17 (m, 2H), 0.01-−0.05 (m, 2H). |
| 207 | 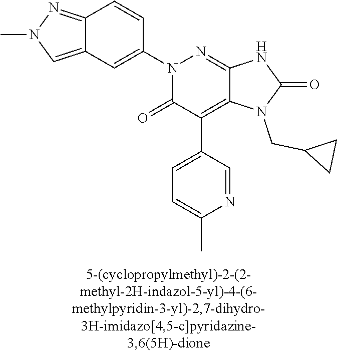 [SMILES: Cc1ccc(-c2c(=O)n(-c3ccc4nn(C)cc4c3)nc3[nH]c(=O)n(CC4CC4)c23)cn1] | LC-MS: m/z 428 (M + H)⁺. ¹H NMR (400 MHz, DMSO- d₆) δ: 11.90 (s, 1H), 8.52 (d, J = 1.8 Hz, 1H), 8.41 (s, 1H), 7.84 (d, J = 1.3 Hz, 1H), 7.81 (dd, J = 7.9, 2.3 Hz, 1H), 7.62 (d, J = 9.2 Hz, 1H), 7.38 (d, J = 8.0 Hz, 1H), 7.34 (dd, J = 9.2, 2.0 Hz, 1H), 4.19 (s, 3H), 3.25 (d, J = 6.9 Hz, 2H), 2.54 (s, 3H), 0.40-0.32 (m, 1H), 0.24-0.19 (m, 2H), 0.03- −0.03 (m, 2H). |

### Example 208: Synthesis of 1-(cyclopropylmethyl)-7-(4-(difluoromethoxy)phenyl)-5-(2-methyl-2H-indazol-5-yl)-1,5-dihydro-6H-[1,2,3]triazolo[4,5-c]pyridazin-6-one via General Procedure VII

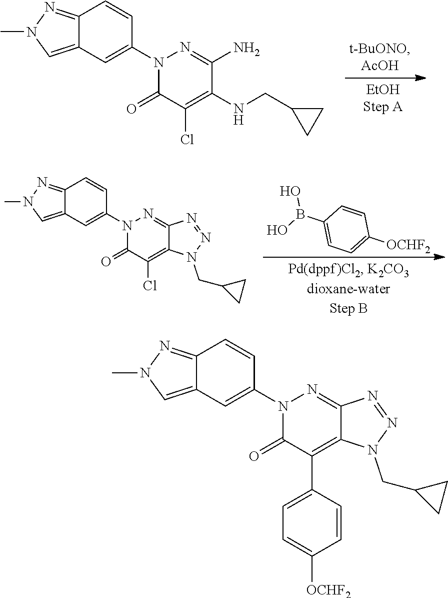

### Step A: 7-chloro-1-(cyclopropylmethyl)-5-(2-methyl-2H-indazol-5-yl)-1,5-dihydro-6H-[1,2,3]triazolo[4,5-c]pyridazin-6-one

A mixture of 6-amino-4-chloro-5-((cyclopropylmethyl)amino)-2-(2-methyl-2H-indazol-5-yl)pyridazin-3(2H)-one (100 mg, 0.29 mmol, 1.0 eq.), tert-butyl nitrite (36 mg, 0.35 mmol, 1.2 eq.) and AcOH (70 mg, 1.1 mmol, 4.0 eq.) in EtOH (2 mL) was sealed in a tube and stirred at 60° C. for 1 hr. After cooling to room temperature, NaHCO₃(86 mg, 1.1 mmol, 3.5 eq.) was added and the resulting mixture was stirred at room temperature for an additional 20 min. The reaction mixture was then diluted with dichloromethane (20 mL), filtered and the filtrate was concentrated under reduced pressure. The resulting residue was purified by column chromatography on silica gel to give 7-chloro-1-(cyclopropylmethyl)-5-(2-methyl-2H-indazol-5-yl)-1,5-dihydro-6H-[1,2,3]triazolo[4,5-c]pyridazin-6-one (100 mg, 97%) as an orange solid. LC-MS (ESI): m/z 356 [M+H]⁺.

### Step B: 1-(cyclopropylmethyl)-7-(4-(difluoromethoxy)phenyl)-5-(2-methyl-2H-indazol-5-yl)-1,5-dihydro-6H-[1,2,3]triazolo[4,5-c]pyridazin-6-one

To a mixture of 7-chloro-1-(cyclopropylmethyl)-5-(2-methyl-2H-indazol-5-yl)-1,5-dihydro-6H-[1,2,3]triazolo[4,5-c]pyridazin-6-one (60 mg, 0.17 mmol, 1.0 eq.), (4-(difluoromethoxy)phenyl)boronic acid (55 mg, 0.21 mmol, 1.2 eq.) and K₂CO₃ (70 mg, 0.51 mmol, 3.0 eq.) in dioxane (2 mL) and H₂O (0.5 mL) was added Pd(dppf)Cl₂ (12 mg, 0.017 mmol, 0.1 eq.), the reaction mixture was degassed with N₂ and stirred at 100° C. under N₂ atmosphere overnight. After completion, the reaction mixture was diluted with H₂O (10 mL) and extracted with EtOAc (15 mL×3). The combined organic layers were washed with brine (10 mL), dried over anhydrous Na₂SO₄ and concentrated under reduced pressure. The resulting residue was purified by flash column chromatography on silica gel and Prep-TLC to give 1-(cyclopropylmethyl)-7-(4-(difluoromethoxy)phenyl)-5-(2-methyl-2H-indazol-5-yl)-1,5-dihydro-6H-[1,2,3]triazolo[4,5-c]pyridazin-6-one (Example 208). ¹H NMR (400 MHz, DMSO-d₆) δ (ppm): 8.51 (s, 1H), 8.03 (d, J=1.4 Hz, 1H), 7.71 (d, J=9.2 Hz, 1H), 7.70-7.67 (m, 2H), 7.46 (dd, J=9.2, 2.0 Hz, 1H), 7.38 (t, J_{HF}=74.0 Hz, 1H), 7.36 (d, J=8.6 Hz, 2H), 4.22 (s, 3H), 4.06 (d, J=7.2 Hz, 2H), 0.70-0.60 (m, 1H), 0.37-0.30 (m, 2H), 0.13-0.06 (m, 2H). LC-MS (ESI): m/z 464 [M+H]⁺.

The procedure set forth above for General Procedure VII was used to synthesize the following compounds by using appropriate starting materials:

| Example | Structure | Characterization |
|---|---|---|
| 209 | 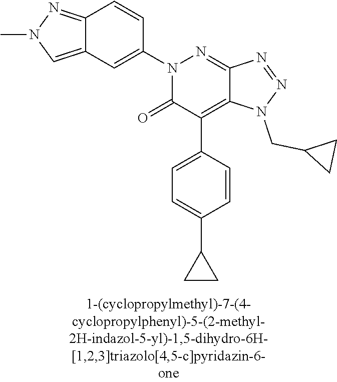 [SMILES: Cn1cc2cc(-n3nc4nnn(CC5CC5)c4c(-c4ccc(C5CC5)cc4)c3=O)ccc2n1] | LC-MS: m/z 438 (M + H)⁺. ¹H NMR (400 MHz, DMSO- d₆) δ: 8.51 (s, 1H), 8.02 (d, J = 1.4 Hz, 1H), 7.70 (d, J = 9.1 Hz, 1H), 7.48 (d, J = 8.2 Hz, 2H), 7.45 (dd, J = 9.2, 2.0 Hz, 1H), 7.25 (d, J = 8.2 Hz, 2H), 4.22 (s, 3H), 4.07 (d, J = 7.5 Hz, 2H), 2.03-2.00 (m, 1H), 1.07-0.98 (m, 2H), 0.81-0.72 (m, 2H), 0.70-0.59 (m, 1H), 0.36-0.27 (m, 2H), 0.07-0.04 (m, 2H). |
| 210 | 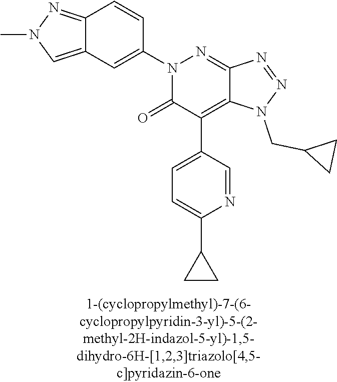 [SMILES: Cn1cc2cc(-n3nc4nnn(CC5CC5)c4c(-c4ccc(C5CC5)nc4)c3=O)ccc2n1] | LC-MS: m/z 439 (M + H)⁺. ¹H NMR (400 MHz, DMSO- d₆) δ: 8.61 (d, J = 2.0 Hz, 1H), 8.51 (s, 1H), 8.03 (d, J = 1.4 Hz, 1H), 7.90 (dd, J = 8.1, 2.2 Hz, 1H), 7.71 (d, J = 9.2 Hz, 1H), 7.49 (d, J = 8.1 Hz, 1H), 7.45 (dd, J = 9.2, 2.0 Hz, 1H), 4.22 (s, 3H), 4.07 (d, J = 7.2 Hz, 2H), 2.25-2.18 (m, 1H), 1.06-1.02 (m, 2H), 1.02-0.99 (m, 2H), 0.73-0.60 (m, 1H), 0.39-0.32 (m, 2H), 0.13-0.09 (m, 2H). |

### Example 211: Synthesis of 1-(cyclopropylmethyl)-7-(6-cyclopropylpyridin-3-yl)-3-methyl-5-(2-methyl-2H-indazol-5-yl)-1H,5H,6H-pyrazolo[4,3-c]pyridazin-6-one via General Procedure VIII

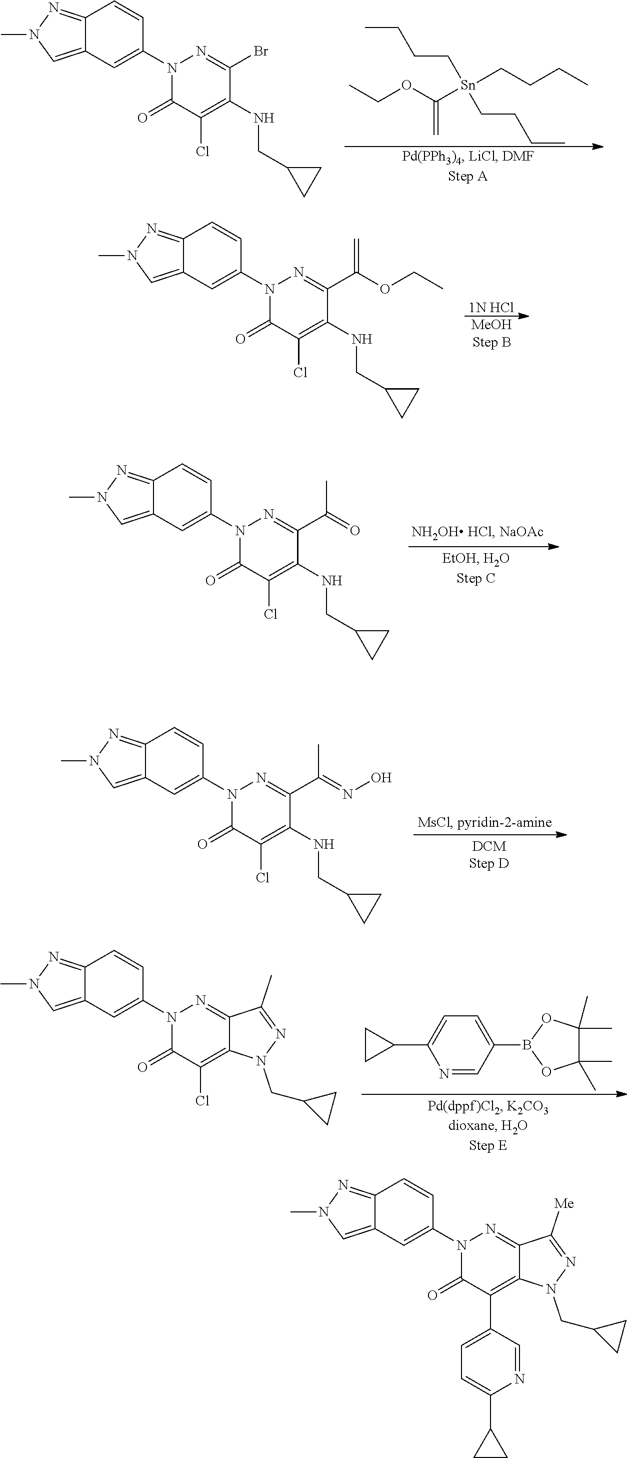

### Step A: 4-chloro-5-((cyclopropylmethyl)amino)-6-(1-ethoxyvinyl)-2-(2-methyl-2H-indazol-5-yl)pyridazin-3(2H)-one

To a solution of 6-bromo-4-chloro-5-((cyclopropylmethyl)amino)-2-(2-methyl-2H-indazol-5-yl)pyridazin-3(2H)-one (1.5 g, 3.67 mmol, 1.0 eq.) in DMF (15 mL) was added Pd(PPh₃)₄ (0.42 g, 0.37 mmol, 0.1 eq.), tributyl(1-ethoxyethenyl)stannane (1.46 g, 4.04 mmol, 1.1 eq.) and lithium chloride (390 mg, 9.18 mmol, 2.5 eq.). The reaction mixture was stirred at 80° C. for 8 hrs. After completion, the reaction mixture was diluted with water (60 mL) and extracted with EtOAc (50 mL×3). The combined organic layers were dried over anhydrous Na₂SO₄, concentrated under reduced pressure, and the residue was purified by flash column chromatography on silica gel to give 4-chloro-5-((cyclopropylmethyl)amino)-6-(1-ethoxyvinyl)-2-(2-methyl-2H-indazol-5-yl)pyridazin-3(2H)-one (950 mg, 65%) as a pale yellow oil. LC-MS (ESI): m/z 400 [M+H]⁺.

### Step B: 6-acetyl-4-chloro-5-((cyclopropylmethyl)amino)-2-(2-methyl-2H-indazol-5-yl)pyridazin-3(2H)-one

To a solution of 4-chloro-5-((cyclopropylmethyl)amino)-6-(1-ethoxyvinyl)-2-(2-methyl-2H-indazol-5-yl)pyridazin-3(2H)-one (950 mg, 2.38 mmol, 1.0 eq.) in MeOH (10 mL) was added 1N HCl (9.5 mL, 9.50 mmol, 4.0 eq.), and the mixture was stirred at 80° C. for 3 hrs. The reaction mixture was quenched with NaHCO₃(sat.aq.) (30 mL) and extracted with EtOAc (50 mL×3). The combined organic layers were dried over anhydrous Na₂SO₄, concentrated under reduced pressure, and the residue was purified by flash column chromatography on silica gel to give 6-acetyl-4-chloro-5-((cyclopropylmethyl)amino)-2-(2-methyl-2H-indazol-5-yl)pyridazin-3(2H)-one (750 mg, 85%) as a pale yellow solid. LC-MS (ESI): m/z 372 [M+H]⁺.

### Step C: (E)-4-chloro-5-((cyclopropylmethyl)amino)-6-(1-(hydroxyimino)ethyl)-2-(2-methyl-2H-indazol-5-yl)pyridazin-3(2H)-one

To a solution of 6-acetyl-4-chloro-5-((cyclopropylmethyl)amino)-2-(2-methyl-2H-indazol-5-yl)pyridazin-3(2H)-one (750 mg, 2.02 mmol, 1.0 eq.) in EtOH (6 mL) was added sodium acetate (496 mg, 6.05 mmol, 3.0 eq.), hydroxylamine hydrochloride (280 mg, 4.03 mmol, 2.0 eq.) and water (1.5 mL). The reaction mixture was stirred at 90° C. for 5 hrs. After completion, the mixture was diluted with water (30 mL) and extracted with EtOAc (50 mL×3). The combined organic layers were dried over anhydrous Na₂SO₄, concentrated under reduced pressure, and the residue was purified by flash column chromatography on silica gel to give (E)-4-chloro-5-((cyclopropylmethyl)amino)-6-(1-(hydroxyimino)ethyl)-2-(2-methyl-2H-indazol-5-yl)pyridazin-3(2H)-one (650 mg, 83%) as a pale yellow solid. LC-MS (ESI): m/z 387 [M+H]⁺.

### Step D: 7-chloro-1-(cyclopropylmethyl)-3-methyl-5-(2-methyl-2H-indazol-5-yl)-1,5-dihydro-6H-pyrazolo[4,3-c]pyridazin-6-one

To a solution of (E)-4-chloro-5-((cyclopropylmethyl)amino)-6-(1-(hydroxyimino)ethyl)-2-(2-methyl-2H-indazol-5-yl)pyridazin-3(2H)-one (650 mg, 1.68 mmol, 1.0 eq.) in DCM (7 mL) was added pyridin-2-amine (316 mg, 3.36 mmol, 2.0 eq.) and methanesulfonyl chloride (388 mg, 3.36 mmol, 2.0 eq.), and the reaction mixture was stirred at 40° C. for 5 hrs. The reaction mixture was quenched with water (40 mL) and extracted with DCM (50 mL×3). The combined organic layers were washed with brine (10 mL), dried over anhydrous Na₂SO₄, concentrated under reduced pressure, and the residue was purified by flash column chromatography on silica gel to give 7-chloro-1-(cyclopropylmethyl)-3-methyl-5-(2-methyl-2H-indazol-5-yl)-1,5-dihydro-6H-pyrazolo[4,3-c]pyridazin-6-one (400 mg, 64%) as a pale yellow solid. LC-MS (ESI): m/z 369 [M+H]⁺.

### Step E: 1-(cyclopropylmethyl)-7-(6-cyclopropylpyridin-3-yl)-3-methyl-5-(2-methyl-2H-indazol-5-yl)-1,5-dihydro-6H-pyrazolo[4,3-c]pyridazin-6-one

To a solution of 7-chloro-1-(cyclopropylmethyl)-3-methyl-5-(2-methyl-2H-indazol-5-yl)-1,5-dihydro-6H-pyrazolo[4,3-c]pyridazin-6-one (50 mg, 0.17 mmol, 1.0 eq.) in dioxane (4 mL) was added 2-cyclopropyl-5-(4,4,5,5-tetramethyl-1,3,2-dioxaborolan-2-yl)pyridine (46 mg, 0.19 mmol, 1.2 eq.), K₂CO₃ (47 mg, 0.34 mmol, 2.0 eq.), Pd(dppf)Cl₂ (10 mg, 0.01 mmol, 0.1 eq.) and water (1 mL). The reaction mixture was stirred at 100° C. under N₂ atmosphere for 5 hrs. The reaction mixture was diluted with water (40 mL), extracted with EtOAc (40 mL×3), the combined organic layers were washed with brine (10 mL), dried over anhydrous Na₂SO₄ and concentrated under reduced pressure. The resulting residue was purified by RP-Prep-TLC to give 1-(cyclopropylmethyl)-7-(6-cyclopropylpyridin-3-yl)-3-methyl-5-(2-methyl-2H-indazol-5-yl)-1H,5H,6H-pyrazolo[4,3-c]pyridazin-6-one (Example 211). ¹H NMR (400 MHz, DMSO-d₆): δ (ppm): 8.51 (d, J=1.8 Hz, 1H), 8.48 (s, 1H), 7.95 (d, J=1.4 Hz, 1H), 7.80 (dd, J=8.0, 2.3 Hz, 1H), 7.68 (d, J=9.1 Hz, 1H), 7.44 (d, J=7.8 Hz, 1H), 7.39 (dd, J=9.1, 2.0 Hz, 1H), 4.21 (s, 3H), 3.55 (d, J=6.9 Hz, 2H), 2.43 (s, 3H), 2.22-2.14 (m, 1H), 1.05-0.94 (m, 4H), 0.77-0.68 (m, 1H), 0.34-0.24 (m, 2H), −0.01-−0.04 (m, 2H). LC-MS (ESI): m/z 452 [M+H]⁺.

The procedure set forth above for General Procedure VIII was used to synthesize the following compounds by using appropriate starting materials:

| Example | Structure | Characterization |
|---|---|---|
| 212 | 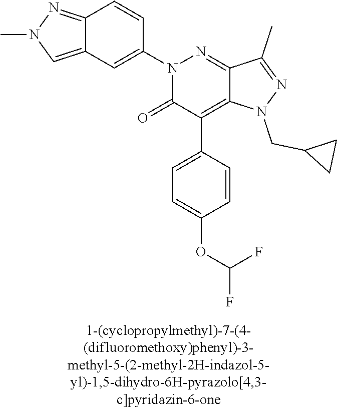 [SMILES: Cc1nn(CC2CC2)c2c(-c3ccc(OC(F)F)cc3)c(=O)n(-c3ccc4nn(C)cc4c3)nc12] | LC-MS: m/z 477 (M + H)⁺. ¹H NMR (400 MHz, DMSO- d₆) δ: 8.48 (s, 1H), 7.95 (d, J = 1.4 Hz, 1H), 7.68 (d, J = 9.1 Hz, 1H), 7.60-7.55 (m, 2H), 7.40 (dd, J = 9.1, 2.0 Hz, 1H), 7.36 (t, J_{HF} = 74 Hz, 1H), 7.31 (d, J = 8.6 Hz, 2H), 4.21 (s, 3H), 3.54 (d, J = 7.0 Hz, 2H), 2.43 (s, 3H), 0.80-0.66 (m, 1H), 0.33-0.22 (m, 2H), −0.04- 0 (m, 2H). |
| 213 | 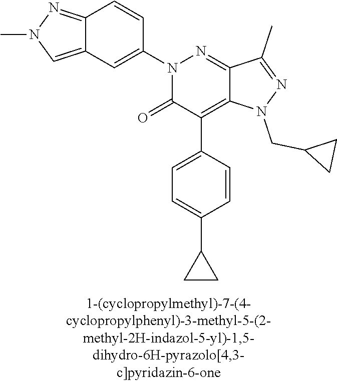 [SMILES: Cc1nn(CC2CC2)c2c(-c3ccc(C4CC4)cc3)c(=O)n(-c3ccc4nn(C)cc4c3)nc12] | LC-MS: m/z 451 (M + H)⁺. ¹H NMR (400 MHz, DMSO- d₆) δ: 8.47 (s, 1H), 7.94 (d, J = 1.4 Hz, 1H), 7.67 (d, J = 9.2 Hz, 1H), 7.42-7.34 (m, 3H), 7.20 (d, J = 8.2 Hz, 2H), 4.21 (s, 3H), 3.53 (d, J = 7.0 Hz, 2H), 2.42 (s, 3H), 2.01-1.97 (m, 1H), 1.06-0.96 (m, 2H), 0.78-0.69 (m, 3H), 0.31-0.20 (m, 2H), −0.03-−0.10 (m, 2H). |
| 214 | 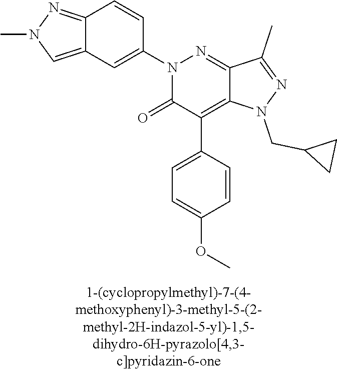 [SMILES: COc1ccc(-c2c(=O)n(-c3ccc4nn(C)cc4c3)nc3c(C)nn(CC4CC4)c23)cc1] | LC-MS: m/z 441 (M + H)⁺. ¹H NMR (400 MHz, DMSO- d₆) δ: 8.47 (s, 1H), 7.94 (d, J = 1.3 Hz, 1H), 7.67 (d, J = 9.1 Hz, 1H), 7.46-7.42 (m, 2H), 7.39 (dd, J = 9.1, 2.0 Hz, 1H), 7.09-7.03 (m, 2H), 4.21 (s, 3H), 3.82 (s, 3H), 3.57 (d, J = 7.0 Hz, 2H), 2.42 (s, 3H), 0.80-0.68 (m, 1H), 0.32-0.23 (m, 2H), −0.02-−0.09 (m, 2H). |
| 215 | 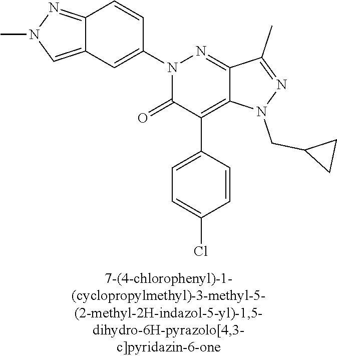 [SMILES: Cc1nn(CC2CC2)c2c(-c3ccc(Cl)cc3)c(=O)n(-c3ccc4nn(C)cc4c3)nc12] | LC-MS: m/z 445 (M + H)⁺. ¹H NMR (400 MHz, DMSO- d₆) δ: 8.48 (s, 1H), 7.95 (d, J = 1.3 Hz, 1H), 7.68 (d, J = 9.2 Hz, 1H), 7.61-7.51 (m, 4H), 7.40 (dd, J = 9.1, 2.0 Hz, 1H), 4.21 (s, 3H), 3.54 (d, J = 7.0 Hz, 2H), 2.43 (s, 3H), 0.79- 0.66 (m, 1H), 0.33-0.24 (m, 2H), 0.00-−0.03 (m, 2H). |
| 216 | 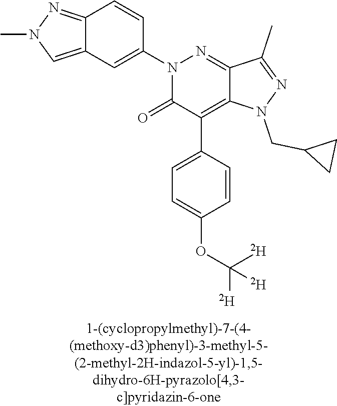 [SMILES: [2H]C([2H])([2H])Oc1ccc(-c2c(=O)n(-c3ccc4nn(C)cc4c3)nc3c(C)nn(CC4CC4)c23)cc1] | LC-MS: m/z 444 (M + H)⁺. ¹H NMR (400 MHz, DMSO- d₆) δ: 8.47 (s, 1H), 7.94 (d, J = 1.4 Hz, 1H), 7.67 (d, J = 9.1 Hz, 1H), 7.44 (d, J = 8.7 Hz, 2H), 7.39 (dd, J = 9.1, 2.0 Hz, 1H), 7.06 (d, J = 8.7 Hz, 2H), 4.21 (s, 3H), 3.57 (d, J = 7.0 Hz, 2H), 2.43 (s, 3H), 0.78- 0.68 (m, 1H), 0.31-0.22 (m, 2H), −0.04-0.0 (m, 2H). |
| 217 | 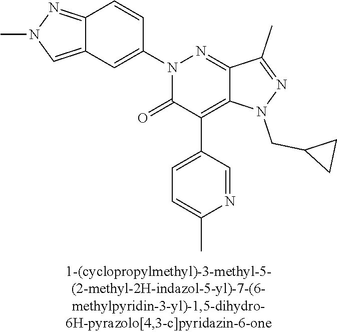 [SMILES: Cc1ccc(-c2c(=O)n(-c3ccc4nn(C)cc4c3)nc3c(C)nn(CC4CC4)c23)cn1] | LC-MS: m/z 426 (M + H)⁺. ¹H NMR (400 MHz, DMSO- d₆) δ: 8.57 (d, J = 1.9 Hz, 1H), 8.48 (s, 1H), 7.95 (d, J = 1.4 Hz, 1H), 7.85 (dd, J = 8.0, 2.3 Hz, 1H), 7.68 (d, J = 9.1 Hz, 1H), 7.44-7.36 (m, 2H), 4.21 (s, 3H), 3.56 (d, J = 7.0 Hz, 2H), 2.55 (s, 3H), 2.44 (s, 3H), 0.78-0.66 (m, 1H), 0.36- 0.24 (m, 2H), −0.03-0.00 (m, 2H). |

### Example 218: Synthesis of 1-(cyclopropylmethyl)-7-(4-(difluoromethoxy)phenyl)-5-(2-methyl-2H-indazol-5-yl)-1,5-dihydro-6H-pyrazolo[4,3-c]pyridazin-6-one via General Procedure IX

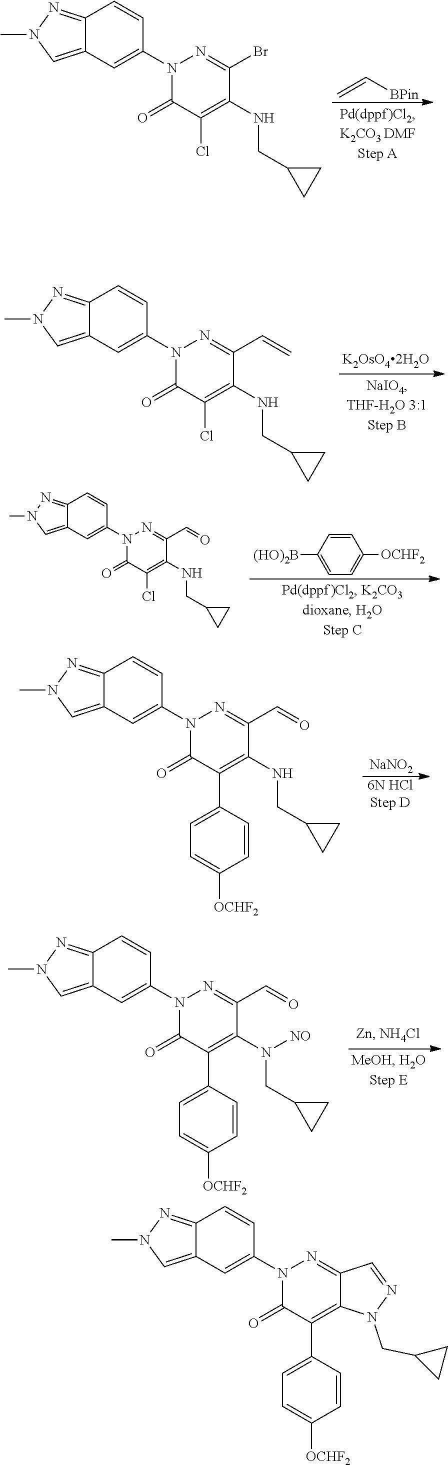

### Step A: 4-chloro-5-((cyclopropylmethyl)amino)-2-(2-methyl-2H-indazol-5-yl)-6-vinylpyridazin-3(2H)-one

A mixture of 6-bromo-4-chloro-5-((cyclopropylmethyl)amino)-2-(2-methyl-2H-indazol-5-yl)pyridazin-3(2H)-one (1 g, 2.447 mmol, 1.0 eq.), 4,4,5,5-tetramethyl-2-vinyl-1,3,2-dioxaborolane (0.5 mL, 2.936 mmol, 1.2 eq.), Pd(dppf)Cl₂ (90 mg, 0.122 mmol, 0.05 eq.) and K₂CO₃ (676 mg, 4.894 mmol, 2.0 eq.) in dioxane (16 mL) and water (4 mL) was stirred at 80° C. under N₂ atmosphere for 18 hrs. The reaction mixture was filtered through a short pad of Celite®, concentrated under reduced pressure, and the residue was purified by flash column chromatography on silica gel to give 4-chloro-5-((cyclopropylmethyl)amino)-2-(2-methyl-2H-indazol-5-yl)-6-vinylpyridazin-3(2H)-one (600 mg, 69%) as a brown oil. LC-MS (ESI): m/z 356 [M+H]⁺.

### Step B: 5-chloro-4-((cyclopropylmethyl)amino)-1-(2-methyl-2H-indazol-5-yl)-6-oxo-1,6-dihydropyridazine-3-carbaldehyde

To a solution of 4-chloro-5-((cyclopropylmethyl)amino)-2-(2-methyl-2H-indazol-5-yl)-6-vinylpyridazin-3(2H)-one (600 mg, 1.686 mmol, 1.0 eq.) in THE (15 mL) and H₂O (5 mL) was added K₂OsO₄.2H₂O (31 mg, 0.084 mmol, 0.05 eq.), the reaction mixture was stirred at 0° C. for 1 hr, then NaIO₄ (1.44 g, 6.745 mmol, 4.0 eq.) was added and the reaction mixture was stirred at room temperature for 12 hrs. After completion, the reaction mixture was quenched with Na₂SO₃ (sat. aq.) (30 mL) and extracted with DCM (40 mL×3). The combined organic layers were washed with brine (10 mL), dried over anhydrous Na₂SO₄ and concentrated under reduced pressure. The resulting residue was purified by flash column chromatography on silica gel to give 5-chloro-4-((cyclopropylmethyl)amino)-1-(2-methyl-2H-indazol-5-yl)-6-oxo-1,6-dihydropyridazine-3-carbaldehyde (400 mg, 66%) as a colorless oil. LC-MS (ESI): m/z 358 [M+H]⁺.

### Step C: 4-((cyclopropylmethyl)amino)-5-(4-(difluoromethoxy)phenyl)-1-(2-methyl-2H-indazol-5-yl)-6-oxo-1,6-dihydropyridazine-3-carbaldehyde

A mixture of 5-chloro-4-((cyclopropylmethyl)amino)-1-(2-methyl-2H-indazol-5-yl)-6-oxo-1,6-dihydropyridazine-3-carbaldehyde (150 mg, 0.419 mmol, 1.0 eq.), (4-(difluoromethoxy)phenyl)boronic acid (95 mg, 0.503 mmol, 1.2 eq.), Pd(dppf)Cl₂ (15 mg, 0.021 mmol, 0.05 eq.) and K₂CO₃ (116 mg, 0.838 mmol, 2 eq.) in dioxane (5 mL) and water (1 mL) was stirred at 100° C. under N₂ atmosphere for 18 hrs. The reaction mixture was diluted with water (30 mL), extracted with DCM (50 mL×2). The combined organic layers were washed with brine (10 mL), dried over anhydrous Na₂SO₄ and concentrated under reduced pressure. The resulting residue was purified by flash column chromatography on silica gel to give 4-((cyclopropylmethyl)amino)-5-(4-(difluoromethoxy)phenyl)-1-(2-methyl-2H-indazol-5-yl)-6-oxo-1,6-dihydropyridazine-3-carbaldehyde (150 mg, 77%) as a brown solid. LC-MS (ESI): m/z 466 [M+H]⁺.

### Step D: N-(cyclopropylmethyl)-N-(5-(4-(difluoromethoxy)phenyl)-3-formyl-1-(2-methyl-2H-indazol-5-yl)-6-oxo-1,6-dihydropyridazin-4-yl)nitrous amide

To a solution of 4-((cyclopropylmethyl)amino)-5-(4-(difluoromethoxy)phenyl)-1-(2-methyl-2H-indazol-5-yl)-6-oxo-1,6-dihydropyridazine-3-carbaldehyde (80 mg, 0.182 mmol, 1.0 eq.) in 4M HCl (4 mL) was added NaNO₂ (63 mg, 0.908 mmol, 5.0 eq.), and the reaction mixture was stirred at room temperature for 2 hrs. The reaction was quenched with Na₂CO₃ (sat. aq.) (50 mL) and extracted with EtOAc (40 mL×3). The combined organic layers were washed with brine (10 mL), dried over anhydrous Na₂SO₄ and concentrated under reduced pressure. The resulting residue was purified by flash column chromatography on silica gel to give N-(cyclopropylmethyl)-N-(5-(4-(difluoromethoxy)phenyl)-3-formyl-1-(2-methyl-2H-indazol-5-yl)-6-oxo-1,6-dihydropyridazin-4-yl)nitrous amide (51 mg, 60%) as a yellow solid. LC-MS (ESI): m/z 495 [M+H]⁺.

### Step E: 1-(cyclopropylmethyl)-7-(4-(difluoromethoxy)phenyl)-5-(2-methyl-2H-indazol-5-yl)-1,5-dihydro-6H-pyrazolo[4,3-c]pyridazin-6-one

To a solution of N-(cyclopropylmethyl)-N-(5-(4-(difluoromethoxy)phenyl)-3-formyl-1-(2-methyl-2H-indazol-5-yl)-6-oxo-1,6-dihydropyridazin-4-yl)nitrous amide (20 mg, 0.043 mmol, 1.0 eq.) in AcOH (2 mL) and DCM (2 mL) was added zinc powder (28 mg, 0.426 mmol, 10 eq.), and the reaction mixture was stirred at room temperature for 1.5 hrs. The reaction was quenched with NaHCO₃(sat. aq.) (20 mL), filtered through a short pad of Celite®, and the filtrate was extracted with EtOAc (20 mL×3). The combined organic layers were washed with brine (10 mL), dried over anhydrous Na₂SO₄, and concentrated under reduced pressure. The resulting residue was purified by flash column chromatography on silica gel and Prep-TLC to give 1-(cyclopropylmethyl)-7-(4-(difluoromethoxy)phenyl)-5-(2-methyl-2H-indazol-5-yl)-1,5-dihydro-6H-pyrazolo[4,3-c]pyridazin-6-one (Example 218). ¹H NMR (400 MHz, DMSO-d₆): δ (ppm) 8.49 (s, 1H), 8.47 (s, 1H), 7.96 (d, J=1.5 Hz, 1H), 7.68 (d, J=9.2 Hz, 1H), 7.60 (d, J=8.7 Hz, 2H), 7.41 (dd, J=9.1, 2.0 Hz, 1H), 7.37 (t, J_{HF}=73.8 Hz, 1H), 7.32 (d, J=8.6 Hz, 2H), 4.21 (s, 3H), 3.62 (d, J=7.1 Hz, 2H), 0.76-0.67 (m, 1H), 0.31-0.26 (m, 2H), 0.11-−0.11 (m, 2H). LC-MS (ESI): m/z 463 [M+H]⁺.

The procedure set forth above for General Procedure IX was used to synthesize the following compounds by using appropriate starting materials:

| Example | Structure | Characterization |
|---|---|---|
| 219 | 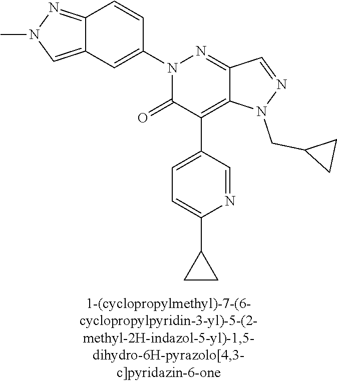 [SMILES: Cn1cc2cc(-n3nc4cnn(CC5CC5)c4c(-c4ccc(C5CC5)nc4)c3=O)ccc2n1] | LC-MS: m/z 438 (M + H)⁺. ¹H NMR (400 MHz, DMSO- d₆) δ: 8.53 (d, J = 1.6 Hz, 1H), 8.48 (d, J = 5.9 Hz, 2H), 7.95 (d, J = 1.3 Hz, 1H), 7.82 (dd, J = 8.0, 2.3 Hz, 1H), 7.67 (d, J = 9.2 Hz, 1H), 7.44 (d, J = 7.4 Hz, 1H), 7.40 (dd, J = 9.2, 2.0 Hz, 1H), 4.20 (s, 3H), 3.64 (d, J = 7.2 Hz, 2H), 2.22-2.19 (m, 1H), 1.03-0.96 (m, 4H), 0.75- 0.69 (m, 1H), 0.33-0.25 (m, 2H), −0.02-0.03 (m, 2H) |
| 220 | 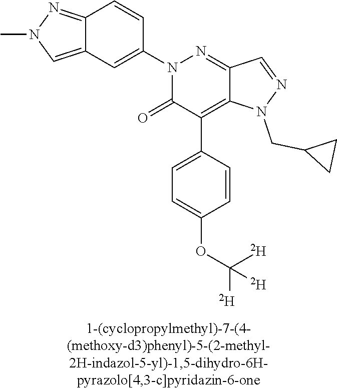 [SMILES: [2H]C([2H])([2H])Oc1ccc(-c2c(=O)n(-c3ccc4nn(C)cc4c3)nc3cnn(CC4CC4)c23)cc1] | LC-MS: m/z 430 (M + H)⁺. ¹H NMR (400 MHz, DMSO- d₆) δ: 8.45 (s, 1H), 8.44 (s, 1H), 7.94 (d, J = 1.3 Hz, 1H), 7.66 (d, J = 9.1 Hz, 1H), 7.45 (d, J = 8.7 Hz, 2H), 7.39 (dd, J = 9.2, 1.9 Hz, 1H), 7.05 (d, J = 8.7 Hz, 2H), 4.19 (s, 3H), 3.64 (d, J = 7.0 Hz, 2H), 0.77- 0.65 (m, 1H), 0.33-0.21 (m, 2H), 0.05-−0.05 (m, 2H). |
| 221 | 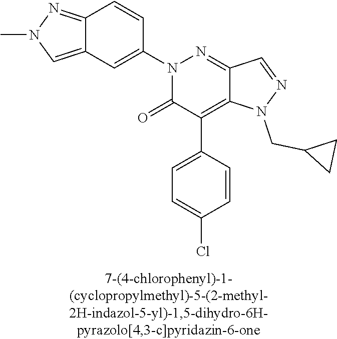 [SMILES: Cn1cc2cc(-n3nc4cnn(CC5CC5)c4c(-c4ccc(Cl)cc4)c3=O)ccc2n1] | LC-MS: m/z 431 (M + H)⁺. ¹H NMR (400 MHz, DMSO- d₆) δ: 8.45 (d, J = 7.4 Hz, 2H), 7.93 (d, J = 1.4 Hz, 1H), 7.65 (d, J = 9.1 Hz, 1H), 7.60-7.51 (m, 4H), 7.38 (dd, J = 9.1, 1.9 Hz, 1H), 4.18 (s, 3H), 3.60 (d, J = 7.0 Hz, 2H), 0.72-0.68 (m, 1H), 0.30-0.25 (m, 2H), 0.01- −0.01 (m, 2H). |
| 222 | 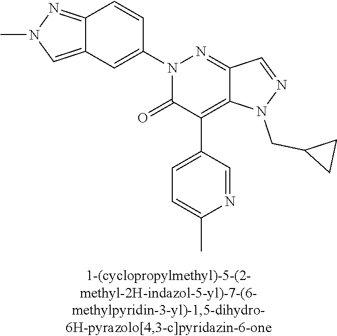 [SMILES: Cc1ccc(-c2c(=O)n(-c3ccc4nn(C)cc4c3)nc3cnn(CC4CC4)c23)cn1] | LC-MS: m/z 412 (M + H)⁺. ¹H NMR (400 MHz, DMSO- d₆) δ: 8.58 (d, J = 1.9 Hz, 1H), 8.49 (s, 1H), 8.46 (s, 1H), 7.96 (d, J = 1.2 Hz, 1H), 7.87 (dd, J = 8.0, 2.3 Hz, 1H), 7.67 (d, J = 9.2 Hz, 1H), 7.41 (d, J = 9.2 Hz, 1H), 7.40 (dd, J = 8.0, 2.3 Hz, 1H), 4.20 (s, 3H), 3.63 (d, J = 7.0 Hz, 2H), 2.55 (s, 3H), 0.76-0.67 (m, 1H), 0.33-0.25 (m, 2H), 0.05- −0.02 (m, 2H). |

### Example 223: Synthesis of 5-(cyclopropylmethyl)-4-(4-(difluoromethoxy)phenyl)-2-(1-methyl-6-oxo-1,6-dihydropyridin-3-yl)-2,5-dihydro-3H-pyrrolo[3,2-c]pyridazin-3-one via General Procedure X (Method A)

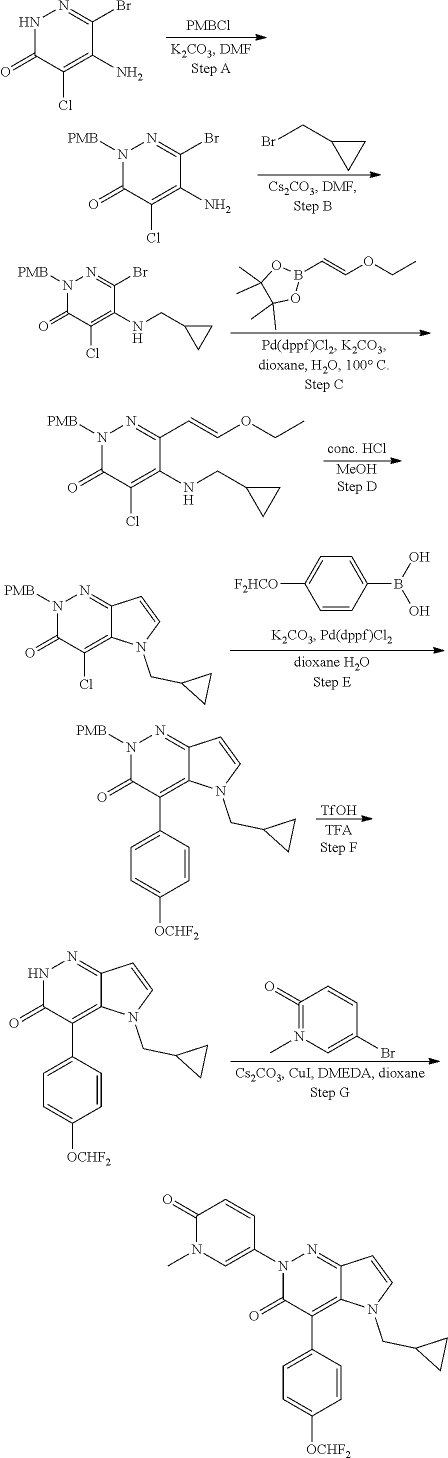

### Step A: 5-amino-6-bromo-4-chloro-2-(4-methoxybenzyl)pyridazin-3(2H)-one

To a solution of 5-amino-6-bromo-4-chloro-2,3-dihydropyridazin-3-one (21 g, 93.6 mmol, 1.0 eq.) in DMF (200 mL) was added K₂CO₃ (25.86 g, 187.1 mmol, 2.0 eq.) and PMBCl (14.51 g, 93.6 mmol, 1.0 eq.), and the reaction mixture was stirred at 80° C. for 5 hrs. After completion, the reaction mixture was carefully poured into 300 mL of ice water, a precipitate formed and the mixture was filtered. The filter cake was washed with water (200 mL) and then dried in vacuum to give 5-amino-6-bromo-4-chloro-2-(4-methoxybenzyl)pyridazin-3(2H)-one (27.2 g, 84%) as a gray solid. LC-MS (ESI): m/z 344 [M+H]⁺.

### Step B: 6-bromo-4-chloro-5-((cyclopropylmethyl)amino)-2-(4-methoxybenzyl)pyridazin-3(2H)-one

To a solution of 5-amino-6-bromo-4-chloro-2-(4-methoxybenzyl)pyridazin-3(2H)-one (6 g, 17.4 mmol, 1.0 eq.) in DMF (60 mL) was added (bromomethyl)cyclopropane (2.39 mL, 24.4 mmol, 1.5 eq.) and Cs₂CO₃ (17.03 g, 52.2 mmol, 2.0 eq.). The reaction mixture was stirred at room temperature under N₂ atmosphere for 6 hrs. After completion, the reaction was diluted with water (80 mL), extracted with DCM (150 mL×3), and the combined organic phase was washed with brine (50 mL), dried over anhydrous Na₂SO₄ and concentrated under reduced pressure. The resulting residue was purified by flash column chromatography on silica gel to give 6-bromo-4-chloro-5-((cyclopropylmethyl)amino)-2-(4-methoxybenzyl)pyridazin-3(2H)-one (5.1 g, 74%) as a white solid. LC-MS (ESI): m/z 398 [M+H]⁺

### Step C: (E)-4-chloro-5-((cyclopropylmethyl)amino)-6-(2-ethoxyvinyl)-2-(4-methoxybenzyl)pyridazin-3(2H)-one

To a solution of 6-bromo-4-chloro-5-((cyclopropylmethyl)amino)-2-(4-methoxybenzyl)pyridazin-3(2H)-one (5.1 g, 12.792 mmol, 1.0 eq.), (E)-2-(2-ethoxyvinyl)-4,4,5,5-tetramethyl-1,3,2-dioxaborolane (3.25 mL, 15.350 mmol, 1.2 eq.) and Pd(dppf)Cl₂ (0.47 g, 0.640 mmol, 0.05 eq.) in dioxane (33 mL) was added a solution of K₂CO₃ (3.54 g, 25.584 mmol, 2.0 eq.) in H₂O (8 mL). The reaction mixture was stirred at 80° C. under N₂ atmosphere for 1.5 hrs. The reaction was diluted with water (40 mL), extracted with EtOAc (60 mL×3), and the combined organic phase was washed with brine (50 mL), dried over anhydrous Na₂SO₄ and concentrated under reduced pressure. The resulting residue was purified by flash column chromatography on silica gel to give (E)-4-chloro-5-((cyclopropylmethyl)amino)-6-(2-ethoxyvinyl)-2-(4-methoxybenzyl)pyridazin-3(2H)-one (4.1 g, 82%) as a white solid. LC-MS (ESI): m/z 390 [M+H]⁺.

### Step D: 4-chloro-5-(cyclopropylmethyl)-2-(4-methoxybenzyl)-2,5-dihydro-3H-pyrrolo[3,2-c]pyridazin-3-one

A solution of (E)-4-chloro-5-((cyclopropylmethyl)amino)-6-(2-ethoxyvinyl)-2-(4-methoxybenzyl)pyridazin-3(2H)-one (4.1 g, 10.516 mmol, 1.0 eq.) in MeOH (40 mL) and concentrated HCl (1 mL) was stirred at 85° C. under N₂ atmosphere for 4 hrs. The reaction mixture was concentrated under reduced pressure, the residue was diluted with H₂O (50 mL), then adjusted pH=7 with solid NaHCO₃. The aqueous layer was extracted with EtOAc (50 mL×3), the combined organic phase was washed with brine (30 mL), dried over anhydrous Na₂SO₄ and concentrated under reduced pressure. The resulting residue was purified by flash column chromatography on silica gel to give 4-chloro-5-(cyclopropylmethyl)-2-(4-methoxybenzyl)-2,5-dihydro-3H-pyrrolo[3,2-c]pyridazin-3-one (3 g, 83%) as a yellow oil. LC-MS (ESI): m/z 344 [M+H]⁺.

### Step E: 5-(cyclopropylmethyl)-4-(4-(difluoromethoxy)phenyl)-2-(4-methoxybenzyl)-2,5-dihydro-3H-pyrrolo[3,2-c]pyridazin-3-one

To a solution of 4-chloro-5-(cyclopropylmethyl)-2-(4-methoxybenzyl)-2,5-dihydro-3H-pyrrolo[3,2-c]pyridazin-3-one (1.5 g, 4.363 mmol, 1.0 eq.), Pd(dppf)Cl₂ (314 mg, 0.436 mmol, 0.1 eq.) and [4-(difluoromethoxy)phenyl]boronic acid (902 mg, 4.799 mmol, 1.1 eq.) in dioxane (16 mL) was added a solution of K₂CO₃ (1.22 g, 8.726 mmol, 2.0 eq.) in H₂O (4 mL). The reaction mixture was stirred at 100° C. under N₂ atmosphere for 2 hrs. The reaction mixture was cooled to room temperature and concentrated under reduced pressure.

The residue was diluted with water (70 mL), the aqueous layer was extracted with DCM (100 mL×3), and the combined organic phase was washed with brine (50 mL), dried over anhydrous Na₂SO₄ and concentrated under reduced pressure. The resulting residue was purified by flash column chromatography on silica gel to give 5-(cyclopropylmethyl)-4-(4-(difluoromethoxy)phenyl)-2-(4-methoxybenzyl)-2,5-dihydro-3H-pyrrolo[3,2-c]pyridazin-3-one (1.1 g, 56%) as a yellow solid. LC-MS (ESI): m/z 452[M+H]⁺.

### Step F: 5-(cyclopropylmethyl)-4-(4-(difluoromethoxy)phenyl)-2,5-dihydro-3H-pyrrolo[3,2-c]pyridazin-3-one

To a solution of 5-(cyclopropylmethyl)-4-(4-(difluoromethoxy)phenyl)-2-(4-methoxybenzyl)-2,5-dihydro-3H-pyrrolo[3,2-c]pyridazin-3-one (1.0 g, 2.215 mmol 1.0 eq.) in trifluoroacetic acid (10 mL) was added trifluoromethanesulfonic acid (3.3 g, 21.988 mmol, 10.0 eq.) at 0° C. The reaction mixture was then stirred at 20° C. for 15 min. The reaction was quenched with NaHCO₃(50 mL) (sat. aq.) at 0° C., the aqueous layer was extracted with DCM (80 mL×3), the combined organic phase was dried over anhydrous Na₂SO₄ and concentrated under reduced pressure. The resulting residue was purified by flash column chromatography on silica gel to give 5-(cyclopropylmethyl)-4-(4-(difluoromethoxy)phenyl)-2,5-dihydro-3H-pyrrolo[3,2-c]pyridazin-3-one (700 mg, 95%) as a yellow solid. LC-MS (ESI): m/z 332 [M+H]⁺.

### Step G: 5-(cyclopropylmethyl)-4-(4-(difluoromethoxy)phenyl)-2-(1-methyl-6-oxo-1,6-dihydropyridin-3-yl)-2,5-dihydro-3H-pyrrolo[3,2-c]pyridazin-3-one

To a solution of 5-(cyclopropylmethyl)-4-(4-(difluoromethoxy)phenyl)-2,5-dihydro-3H-pyrrolo[3,2-c]pyridazin-3-one (50 mg, 0.151 mmol, 1.0 eq.) in dioxane (2 mL) was added Cs₂CO₃ (148 mg, 0.453 mmol, 3.0 eq.), N₁,N₂-dimethylethane-1,2-diamine (27 mg, 0.302 mmol, 2.0 eq.), 5-bromo-1-methyl-1,2-dihydropyridin-2-one (28 mg, 0.151 mmol, 1.2 eq.) and CuI (29 mg, 0.151 mmol, 1.0 eq.). The reaction mixture was degassed with N₂, sealed and heated at 100° C. under microwave irradiation for 1.0 hr. The reaction was quenched with conc. NH₄₀H (20 mL), the aqueous layer was extracted with DCM (20 mL×3), and the combined organic phase was dried over anhydrous Na₂SO₄ and concentrated under reduced pressure. The resulting residue was purified by flash column chromatography on silica gel and Rp-prep-HPLC to give 5-(cyclopropylmethyl)-4-(4-(difluoromethoxy)phenyl)-2-(1-methyl-6-oxo-1,6-dihydropyridin-3-yl)-2,5-dihydro-3H-pyrrolo[3,2-c]pyridazin-3-one. ¹H NMR (400 MHz, DMSO-d₆) δ (ppm): 8.14 (d, J=2.8 Hz, 1H), 7.70 (d, J=3.8 Hz, 1H), 7.67 (dd, J=9.7, 2.9 Hz, 1H), 7.50 (d, J=4.8 Hz, 2H), 7.35 (t, J_{HF}=73.6 Hz, 1H), 7.28 (d, J=8.6 Hz, 2H), 6.44 (d, J=5.6 Hz, 1H), 6.42 (s, 1H), 3.48 (s, 3H), 3.30 (d, J=7.1 Hz, 2H), 0.67-0.56 (m, 1H), 0.35-0.23 (m, 2H), 0.06-0.02 (m, 2H). LC-MS (ESI): m/z 439[M+H]⁺.

### Example 224: Synthesis of 2-(benzo[d]thiazol-6-yl)-5-(cyclopropylmethyl)-4-(4-(difluoromethoxy)phenyl)-2,5-dihydro-3H-pyrrolo[3,2-c]pyridazin-3-one via General Procedure X (Method B)

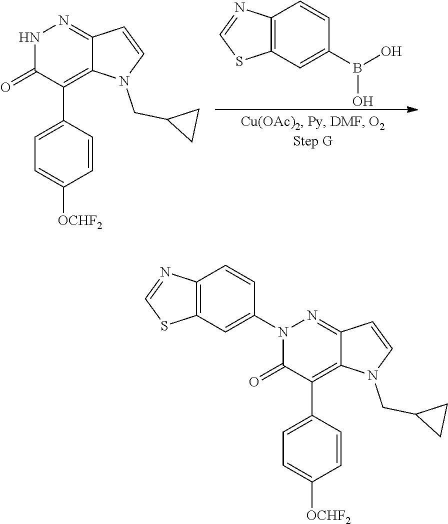 [SMILES: O=c1[nH]nc2ccn(CC3CC3)c2c1-c1ccc(OC(F)F)cc1 ; OB(O)c1ccc2ncsc2c1 ; O=c1c(-c2ccc(OC(F)F)cc2)c2c(ccn2CC2CC2)nn1-c1ccc2ncsc2c1]

### Step G: 2-(benzo[d]thiazol-6-yl)-5-(cyclopropylmethyl)-4-(4-(difluoromethoxy)phenyl)-2,5-dihydro-3H-pyrrolo[3,2-c]pyridazin-3-one

To a solution of 5-(cyclopropylmethyl)-4-(4-(difluoromethoxy)phenyl)-2,5-dihydro-3H-pyrrolo[3,2-c]pyridazin-3-one (50 mg, 0.151 mmol, 1.0 eq.) and (1,3-benzothiazol-6-yl)boronic acid (41 mg, 0.227 mmol, 1.5 eq.) in N,N-dimethylformamide (2 mL) was added Cu(OAc)₂ (55 mg, 0.302 mmol, 2.0 eq.) and pyridine (0.024 mL, 0.302 mmol, 2.0 eq.). The reaction mixture was stirred at 80° C. under 02 atmosphere for 16 hrs. The reaction was completed as indicated by TLC (DCM:MeOH=10:1). The reaction mixture was diluted with H₂O (30 mL) and extracted with EtOAc (40 mL×2). The combined organic layers were washed with brine (30 mL), dried over Na₂SO₄, concentrated under reduced pressure, and the residue was purified by flash chromatography on silica gel and Rp-prep-TLC to give 2-(benzo[d]thiazol-6-yl)-5-(cyclopropylmethyl)-4-(4-(difluoromethoxy)phenyl)-2,5-dihydro-3H-pyrrolo[3,2-c]pyridazin-3-one.

¹H NMR (400 MHz, DMSO-d₆) δ (ppm): 9.43 (s, 1H), 8.39 (d, J=2.0 Hz, 1H), 8.12 (d, J=8.7 Hz, 1H), 7.72-7.67 (m, 2H), 7.50 (d, J=8.6 Hz, 2H), 7.31 (t, J_{HF}=73.6 Hz, 1H), 7.24 (d, J=8.6 Hz, 2H), 6.41 (d, J=3.8 Hz, 1H), 3.27 (d, J=7.1 Hz, 2H), 0.71-0.56 (m, 1H), 0.29-0.19 (m, 2H), 0.03-0.01 (m, 2H).

LC-MS (ESI): m/z 465[M+H]⁺

The procedure set forth above for General Procedure X (Method A) was used to synthesize the following compounds by using appropriate starting materials:

| Example | Structure | Characterization |
|---|---|---|
| 225 | 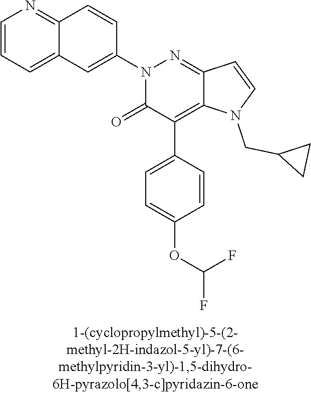 [SMILES: O=c1c(-c2ccc(OC(F)F)cc2)c2c(ccn2CC2CC2)nn1-c1ccc2ncccc2c1] | LC-MS: m/z 459 (M + H)⁺. ¹H NMR (400 MHz, DMSO- d₆) δ: 8.96 (dd, J = 4.2, 1.7 Hz, 1H), 8.47 (d, J = 7.2 Hz, 1H), 8.28 (d, J = 2.3 Hz, 1H), 8.10 (d, J = 9.0 Hz, 1H), 8.00 (dd, J = 9.0, 2.3 Hz, 1H), 7.74 (d, J = 3.8 Hz, 1H), 7.61-7.56 (m, 3H), 7.29 (d, J = 8.6 Hz, 2H), 7.35 (t, J_{HF} = 74 Hz, 1H), 6.48 (d, J = 3.8 Hz, 1H), 3.34 (s, 2H), 0.83-0.51 (m, 1H), 0.44-0.12 (m, 2H), 0.10-0.05 (m, 2H). |
| 226 | 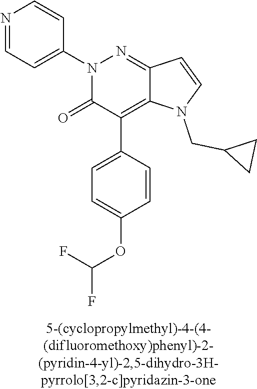 [SMILES: O=c1c(-c2ccc(OC(F)F)cc2)c2c(ccn2CC2CC2)nn1-c1ccncc1] | LC-MS: m/z 409 (M + H)⁺. ¹H NMR (400 MHz, DMSO- d₆) δ: 8.69 (dd, J = 4.7, 1.6 Hz, 2H), 7.85-7.70 (m, 2H), 7.87-7.43 (m, 3H), 7.60-7.47 (m, 2H), 7.35 (t, J_{HF} = 74 Hz, 1H), 6.46 (d, J = 3.8 Hz, 1H), 3.29 (d, J = 7.1 Hz, 2H), 0.71- 0.64 (m, 1H), 0.41-0.23 (m, 2H), 0.07-0.01 (m, 2H). |
| 227 | 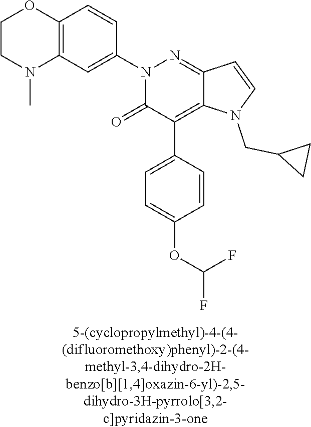 [SMILES: CN1CCOc2ccc(-n3nc4ccn(CC5CC5)c4c(-c4ccc(OC(F)F)cc4)c3=O)cc21] | LC-MS: m/z 479 (M + H)⁺. ¹H NMR (400 MHz, DMSO- d₆) δ: 7.66 (d, J = 4.0 Hz, 1H), 7.55-7.47 (m, 2H), 7.34 (t, J_{HF}= 74 Hz, 1H), 7.27 (d, J = 8.4 Hz, 2H), 6.82 (d, J = 2.4 Hz, 1H), 6.76-6.66 (m, 2H), 6.40 (d, J = 4.0 Hz, 1H), 4.35-4.11 (m, 2H), 3.29-3.23 (m, 4H), 2.82 (s, 3H), 0.71-0.61 (m, 1H), 0.31-0.25 (m, 2H), 0.09- 0.01 (m, 2H). |
| 228 | 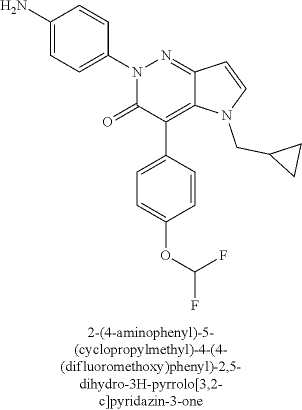 [SMILES: Nc1ccc(-n2nc3ccn(CC4CC4)c3c(-c3ccc(OC(F)F)cc3)c2=O)cc1] | LC-MS: m/z 423 (M + H)⁺. ¹H NMR (400 MHz, DMSO- d₆) δ: 7.64 (d, J = 3.6 Hz, 1H), 7.47 (d, J = 8.8 Hz, 2H), 7.34 (t, J_{HF} = 72 Hz, 1H), 7.26 (d, J = 8.8 Hz, 2H), 7.19-7.13 (m, 2H), 6.59 (d, J = 8.8 Hz, 2H), 6.39 (d, J = 4.0 Hz, 1H), 5.29 (s, 2H), 3.28 (d, J = 7.2 Hz, 2H), 0.71-0.61 (m, 1H), 0.31- 0.25 (m, 2H), 0.09-0.01 (m, 2H). |
| 229 | 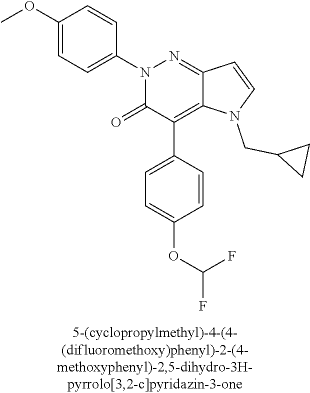 [SMILES: COc1ccc(-n2nc3ccn(CC4CC4)c3c(-c3ccc(OC(F)F)cc3)c2=O)cc1] | LC-MS: m/z 438 (M + H)⁺. ¹H NMR (400 MHz, DMSO- d₆) δ: 7.67 (d, J = 3.8 Hz, 1H), 7.57-7.45 (m, 4H), 7.34 (t, J_{HF} = 74 Hz, 1H), 7.27 (d, J = 8.6 Hz, 2H), 7.06-6.98 (m, 2H), 6.42 (d, J = 3.8 Hz, 1H), 3.80 (s, 3H), 3.29 (d, J = 7.1 Hz, 2H), 0.68-0.64 (m, 1H), 0.36-0.15 (m, 2H), 0.06-0.02 (m, 2H). |
| 230 | 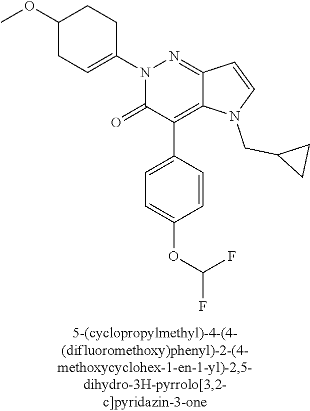 [SMILES: COC1CC=C(n2nc3ccn(CC4CC4)c3c(-c3ccc(OC(F)F)cc3)c2=O)CC1] | LC-MS: m/z 442 (M + H)⁺. ¹H NMR (400 MHz, DMSO- d₆) δ: 7.63 (d, J = 3.8 Hz, 1H), 7.47 (d, J = 8.6 Hz, 2H), 7.33 (t, J_{HF} = 70 Hz, 1H), 7.25 (d, J = 8.6 Hz, 2H), 6.38 (d, J = 3.8 Hz, 1H), 5.75 (s, 1H), 3.54- 3.50 (m, 2H), 3.29 (s, 3H), 3.26 (d, J = 7.1 Hz, 2H), 2.40 (s, 2H), 2.21-2.09 (m, 1H), 2.04-1.87 (m, 1H), 1.74-1.70 (m, 1H), 0.64-0.59 (m, 1H), 0.34-0.16 (m, 2H), 0.04-0.01 (m, 2H). |
| 231 | 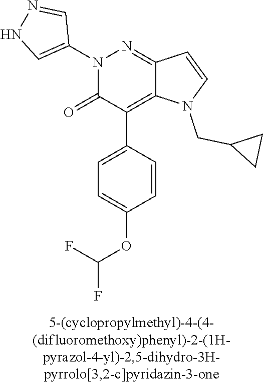 [SMILES: O=c1c(-c2ccc(OC(F)F)cc2)c2c(ccn2CC2CC2)nn1-c1cn[nH]c1] | LC-MS: m/z 398 (M + H)⁺. ¹H NMR (400 MHz, DMSO- d₆) δ: 12.97 (s, 1H), 8.36 (s, 1H), 8.07 (s, 1H), 7.71 (d, J = 3.8 Hz, 1H), 7.52 (d, J = 8.6 Hz, 2H), 7.36 (t, J_{HF} = 73.6 Hz, 1H), 7.29 (d, J = 8.5 Hz, 2H), 6.46 (d, J = 3.7 Hz, 1H), 3.28 (d, J = 7.1 Hz, 2H), 0.70- 0.64 (m, 1H), 0.32-0.22 (m, 2H), 0.06-0.02 (m, 2H). |
| 232 | 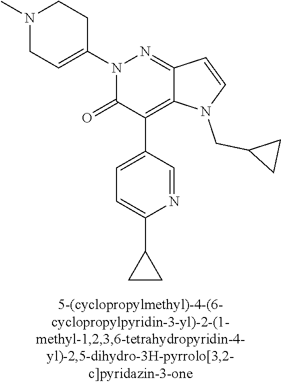 [SMILES: CN1CC=C(n2nc3ccn(CC4CC4)c3c(-c3ccc(C4CC4)nc3)c2=O)CC1] | LC-MS: m/z 402 (M + H)⁺. ¹H NMR (400 MHz, DMSO- d₆) δ: 8.40 (d, J = 2.0 Hz, 1H), 7.70 (dd, J = 8.0, 2.2 Hz, 1H), 7.64 (d, J = 3.8 Hz, 1H), 7.39 (d, J = 8.0 Hz, 1H), 6.39 (d, J = 3.8 Hz, 1H), 5.91 (s, 1H), 3.28 (d, J = 7.1 Hz, 2H), 3.04- 3.02 (m, 2H), 2.60-2.57 (m, 2H), 2.51-2.47 (m, 2H), 2.29 (s, 3H), 2.21-2.14 (m, 1H), 1.05-0.94 (m, 4H), 0.62-0.60 (m, 1H), 0.29-0.24 (m, 2H), 0.06-0.01 (m, 2H). |

### Example 233: Synthesis of 7-chloro-5-(cyclopropylmethyl)-4-(6-cyclopropylpyridin-3-yl)-2-(2-methyl-2H-indazol-5-yl)-2,5-dihydro-3H-pyrrolo[3,2-c]pyridazin-3-one

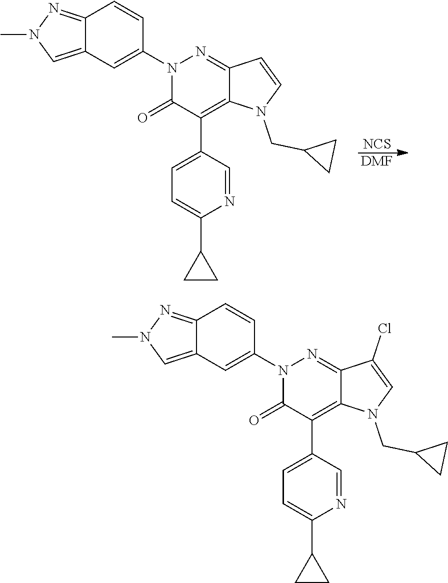

To a solution of 5-(cyclopropylmethyl)-4-(6-cyclopropylpyridin-3-yl)-2-(2-methyl-2H-indazol-5-yl)-2,5-dihydro-3H-pyrrolo[3,2-c]pyridazin-3-one (Example 106) (60 mg, 0.137 mmol, 1.0 eq.) in DMF (3 mL) was added NCS (28 mg, 0.206 mmol, 1.5 eq.). The reaction was stirred at 50° C. under N₂ atmosphere overnight. After completion, the reaction was quenched with ice water (12 ml), and the aqueous layer was extracted with EtOAc (30 mL×3). The combined organic layers were washed with brine (10 mL), dried over Na₂SO₄ and concentrated under reduced pressure. The resulting residue was purified by RP-Prep-HPLC to give 7-chloro-5-(cyclopropylmethyl)-4-(6-cyclopropylpyridin-3-yl)-2-(2-methyl-2H-indazol-5-yl)-2,5-dihydro-3H-pyrrolo[3,2-c]pyridazin-3-one (Example 233). ¹H NMR (400 MHz, DMSO-d₆) δ (ppm): 8.49 (s, 1H), 8.45 (s, 1H), 7.97 (s, 1H), 7.91 (s, 1H), 7.79 (d, J=8.4 Hz, 1H), 7.66 (d, J=9.2 Hz, 1H), 7.42 (d, J=8.0 Hz, 1H), 7.37 (d, J=9.2 Hz, 1H), 4.21 (s, 3H), 3.30 (d, J=7.1 Hz, 2H), 2.20-2.14 (m, 1H), 1.06-0.93 (m, 4H), 0.74-0.53 (m, 1H), 0.35-0.28 (m, 2H), 0.10-0.06 (m, 2H). LC-MS (ESI): m/z 471 [M+H]⁺.

### Example 234: Synthesis of 5-(cyclopropylmethyl)-4-(6-cyclopropylpyridin-3-yl)-7-methyl-2-(2-methyl-2H-indazol-5-yl)-2,5-dihydro-3H-pyrrolo[3,2-c]pyridazin-3-one

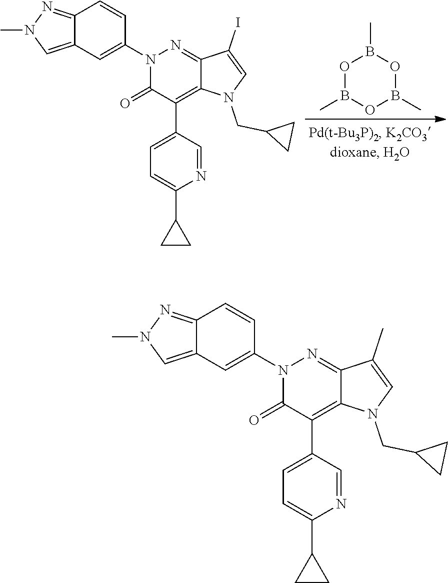

To a solution of 5-(cyclopropylmethyl)-4-(6-cyclopropylpyridin-3-yl)-7-iodo-2-(2-methyl-2H-indazol-5-yl)-2,5-dihydro-3H-pyrrolo[3,2-c]pyridazin-3-one (Example 117) (90 mg, 0.16 mmol, 1.0 eq.), 2,4,6-trimethyl-1,3,5,2,4,6-trioxatriborinane (26 mg, 0.28 mmol, 1.3 eq.) and Pd(tBu₃P)₂ (8 mg, 0.016 mmol, 0.1 eq.) in dioxane (1.6 mL) was added a solution of K₂CO₃ (44 mg, 0.32 mmol, 2.0 eq.) in H₂O (0.4 mL). The reaction mixture was degassed with N₂, sealed in a tube, and heated under microwave irradiation at 100° C. for 1 hr. Then the reaction mixture cooled to room temperature and diluted H₂O (20 mL). The aqueous layer was extracted with EtOAc (20 mL×3). The combined organic layers were washed with brine (20 mL), dried over Na₂SO₄ and concentrated under reduced pressure. The resulting residue was purified by flash column chromatography on silica gel and RP-Prep-HPLC to give 5-(cyclopropylmethyl)-4-(6-cyclopropylpyridin-3-yl)-7-methyl-2-(2-methyl-2H-indazol-5-yl)-2,5-dihydro-3H-pyrrolo[3,2-c]pyridazin-3-one.

¹H NMR (400 MHz, DMSO-d₆) δ (ppm): 8.45 (d, J=1.9 Hz, 1H), 8.43 (s, 1H), 7.88 (d, J=1.4 Hz, 1H), 7.74 (dd, J=8.0, 2.2 Hz, 1H), 7.64 (d, J=9.1 Hz, 1H), 7.44-7.34 (m, 3H), 4.20 (s, 3H), 3.26 (d, J=7.0 Hz, 2H), 2.21-2.14 (m, 1H), 2.09 (s, 3H), 1.01-0.95 (m, 4H), 0.72-0.60 (m, 1H), 0.39-0.22 (m, 2H), 0.10-0.01 (m, 2H).

LC-MS (ESI): m/z 451 [M+H]⁺.

### Example 235: Synthesis of 5-(cyclopropylmethyl)-4-(6-cyclopropylpyridin-3-yl)-7-fluoro-2-(2-methyl-2H-indazol-5-yl)-2,5-dihydro-3H-pyrrolo[3,2-c]pyridazin-3-one

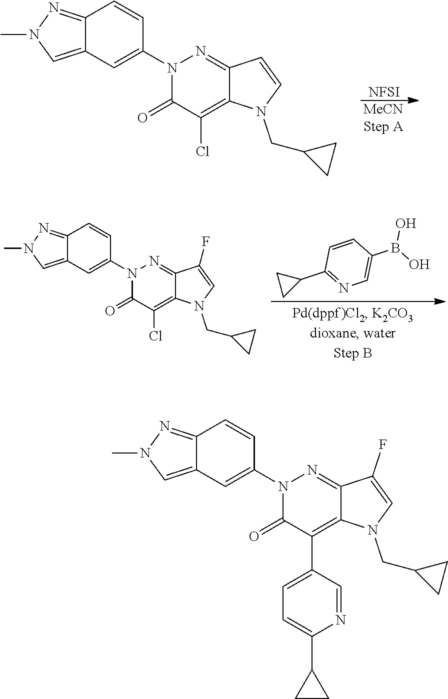

### Step A: 4-chloro-5-(cyclopropylmethyl)-7-fluoro-2-(2-methyl-2H-indazol-5-yl)-2,5-dihydro-3H-pyrrolo[3,2-c]pyridazin-3-one

To a solution of 4-chloro-5-(cyclopropylmethyl)-2-(2-methyl-2H-indazol-5-yl)-2,5-dihydro-3H-pyrrolo[3,2-c]pyridazin-3-one (200 mg, 0.57 mmol, 1.0 eq.) in MeCN (8 mL) was added NFSI (231 mg, 0.74 mmol, 1.3 eq.), and the reaction mixture was stirred at 85° C. under N₂ atmosphere for 15 hrs. The resulting mixture was cooled to room temperature, poured into ice water (15 mL), extracted with DCM (30 mL×3), and the combined organic layers were washed with brine (20 mL), dried over anhydrous Na₂SO₄ and concentrated under reduced pressure. The resulting residue was purified by flash column chromatography on silica gel to give 4-chloro-5-(cyclopropylmethyl)-7-fluoro-2-(2-methyl-2H-indazol-5-yl)-2,5-dihydro-3H-pyrrolo[3,2-c]pyridazin-3-one (110 mg, 42%) as a yellow solid. LC-MS (ESI): m/z 372 [M+H]⁺.

### Step B: 5-(cyclopropylmethyl)-4-(6-cyclopropylpyridin-3-yl)-7-fluoro-2-(2-methyl-2H-indazol-5-yl)-2,5-dihydro-3H-pyrrolo[3,2-c]pyridazin-3-one

To a solution of 4-chloro-5-(cyclopropylmethyl)-7-fluoro-2-(2-methyl-2H-indazol-5-yl)-2,5-dihydro-3H-pyrrolo[3,2-c]pyridazin-3-one (100 mg, 0.22 mmol, 1.0 eq.), (6-cyclopropylpyridin-3-yl)boronic acid (52 mg, 0.32 mmol, 1.5 eq.) in dioxane/H₂O (4 mL, 4:1, v/v) was added K₂CO₃ (74 mg, 0.54 mmol, 2.5 eq.) and Pd(dppf)Cl₂ (15.7 mg, 0.022 mmol, 0.1 eq.) and stirred at 100° C. for 3 hrs. The resulting mixture was concentrated under reduced pressure, and the residue was purified by flash column chromatography on silica gel and Prep-TLC to give 5-(cyclopropylmethyl)-4-(6-cyclopropylpyridin-3-yl)-7-fluoro-2-(2-methyl-2H-indazol-5-yl)-2,5-dihydro-3H-pyrrolo[3,2-c]pyridazin-3-one (Example 235). ¹H NMR (400 MHz, DMSO-d₆) δ (ppm): 8.48 (d, J=2.0 Hz, 1H), 8.45 (s, 1H), 7.92 (d, J=1.2 Hz, 1H), 7.81 (d, J=2.0 Hz, 1H), 7.78 (dd, J=8.0, 2.0 Hz, 1H), 7.65 (d, J=9.2 Hz, 1H), 7.42 (d, J=8.0 Hz, 1H), 7.38 (dd, J=9.2, 2.0 Hz, 1H), 4.21 (s, 3H), 3.24 (d, J=7.2 Hz, 2H), 2.22-2.15 (m, 1H), 1.02-0.97 (m, 4H), 0.74-0.64 (m, 1H), 0.39-0.27 (m, 2H), 0.13-0.04 (m, 2H). LC-MS (ESI): m/z 455 [M+H]⁺.

### Example 236: Synthesis of 7-acetyl-5-(cyclopropylmethyl)-4-(6-cyclopropylpyridin-3-yl)-2-(2-methyl-2H-indazol-5-yl)-2,5-dihydro-3H-pyrrolo[3,2-c]pyridazin-3-one

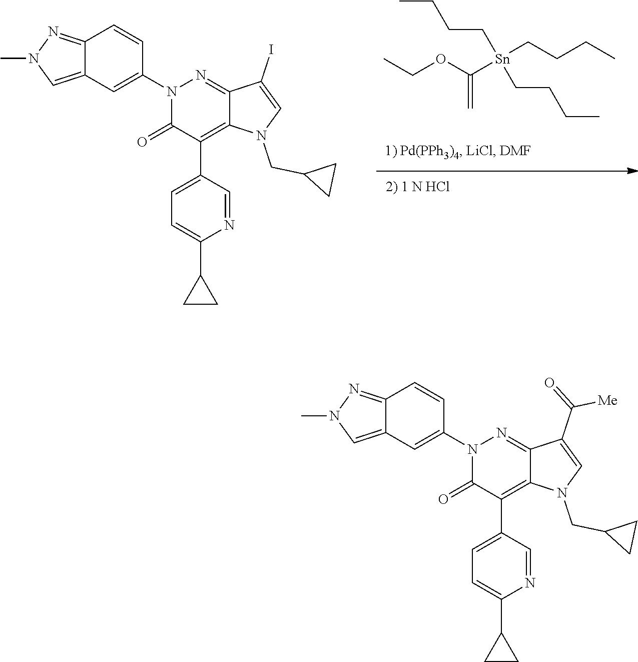 [SMILES: Cn1cc2cc(-n3nc4c(I)cn(CC5CC5)c4c(-c4ccc(C5CC5)nc4)c3=O)ccc2n1 ; C=[C](OCC)[Sn]([CH2]CCC)([CH2]CCC)[CH2]CCC ; CC(=O)c1cn(CC2CC2)c2c(-c3ccc(C4CC4)nc3)c(=O)n(-c3ccc4nn(C)cc4c3)nc12]

To a degassed suspension of 5-(cyclopropylmethyl)-4-(6-cyclopropylpyridin-3-yl)-7-iodo-2-(2-methyl-2H-indazol-5-yl)-2,5-dihydro-3H-pyrrolo[3,2-c]pyridazin-3-one (200 mg, 0.35 mmol, 1.0 eq.) and LiCl (38 mg, 0.9 mmol, 2.5 eq.) in DMF (5 mL) was added tributyl(1-ethoxyethenyl)stannane (193 mg, 0.533 mmol, 1.5 eq.) and Pd(PPh₃)₄ (41 mg, 0.035 mmol, 0.1 eq.), and the resulting mixture was stirred at 80° C. for 18 hrs. Hydrochloric acid solution (10 mL, 1N) was then added to the reaction mixture and stirred for 1 hour at room temperature. The mixture was extracted with EtOAc (10 mL×3) and the combined organic layers were washed with brine (10 mL), dried over Na₂SO₄, filtered and concentrated under reduced pressure. The resulting residue was purified by flash column chromatography on silica gel to give 7-acetyl-5-(cyclopropylmethyl)-4-(6-cyclopropylpyridin-3-yl)-2-(2-methyl-2H-indazol-5-yl)-2,5-dihydro-3H-pyrrolo[3,2-c]pyridazin-3-one.

¹H NMR (400 MHz, DMSO-d₆) δ (ppm): 8.55 (s, 1H), 8.49 (d, J=1.8 Hz, 1H), 8.46 (s, 1H), 7.95 (d, J=1.4 Hz, 1H), 7.80 (dd, J=8.0, 2.2 Hz, 1H), 7.67 (d, J=9.1 Hz, 1H), 7.47-7.38 (m, 2H), 4.21 (s, 3H), 3.41 (d, J=7.1 Hz, 2H), 2.49 (s, 3H), 2.24-2.15 (m, 1H), 1.06-0.94 (m, 4H), 0.77-0.65 (m, 1H), 0.37-0.30 (m, 2H), 0.19-0.11 (m, 2H). LC-MS (ESI): m/z 479 [M+H]⁺.

### Example 237: Synthesis of 5-(cyclopropylmethyl)-7-fluoro-2-(2-methyl-2H-indazol-5-yl)-4-(6-methylpyridin-3-yl)-2,5-dihydro-3H-pyrrolo[3,2-c]pyridazin-3-one

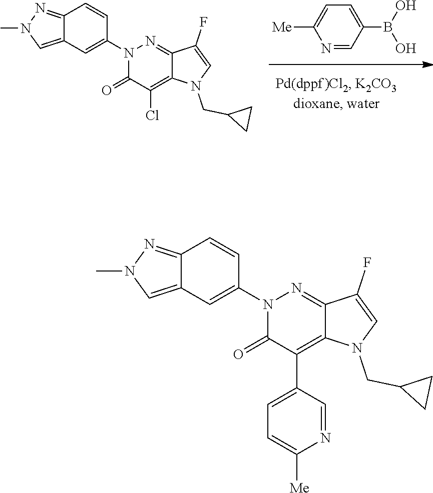 [SMILES: Cn1cc2cc(-n3nc4c(F)cn(CC5CC5)c4c(Cl)c3=O)ccc2n1 ; Cc1ccc(B(O)O)cn1 ; Cc1ccc(-c2c(=O)n(-c3ccc4nn(C)cc4c3)nc3c(F)cn(CC4CC4)c23)cn1]

To a mixture of 4-chloro-5-(cyclopropylmethyl)-7-fluoro-2-(2-methyl-2H-indazol-5-yl)-2,5-dihydro-3H-pyrrolo[3,2-c]pyridazin-3-one (55 mg, 0.15 mmol, 1.0 eq.), (6-methylpyridin-3-yl)boronic acid (31 mg, 0.22 mmol, 1.5 eq.), K₂CO₃ (62 mg, 0.45 mmol, 3.0 eq.) in dioxane (2 mL) and H₂O (0.5 mL) was added Pd(dppf)Cl₂ (11 mg, 0.015 mmol, 0.1 eq.). The reaction mixture was degassed with N₂, sealed in a tube, and heated under microwave irradiation at 100° C. for 1.5 hours. The reaction mixture was diluted with H₂O (10 mL) and extracted with EtOAc (10 mL×3). The combined organic layers was washed with brine (10 mL), dried over Na₂SO₄ and concentrated under reduced pressure. The resulting residue was purified by flash column chromatography on silica gel and RP-Prep-HPLC to give 5-(cyclopropylmethyl)-7-fluoro-2-(2-methyl-2H-indazol-5-yl)-4-(6-methylpyridin-3-yl)-2,5-dihydro-3H-pyrrolo[3,2-c]pyridazin-3-one.

¹H NMR (400 MHz, DMSO-d₆) δ (ppm): 8.54 (d, J=1.9 Hz, 1H), 8.45 (s, 1H), 7.92 (d, J=1.4 Hz, 1H), 7.83 (dd, J=8.4, 2.0 Hz, 2H), 7.66 (d, J=9.1 Hz, 1H), 7.41-7.36 (m, 2H), 4.21 (s, 3H), 3.24 (d, J=7.1 Hz, 2H), 2.55 (s, 3H), 0.74-0.66 (m, 1H), 0.36-0.29 (m, 2H), 0.16-0.05 (m, 2H). LC-MS (ESI): m/z 429 [M+H]⁺.

### Example 238: Synthesis of 7-bromo-5-(cyclopropylmethyl)-4-(6-cyclopropylpyridin-3-yl)-2-(2-methyl-2H-indazol-5-yl)-2,5-dihydro-3H-pyrrolo[3,2-c]pyridazin-3-one

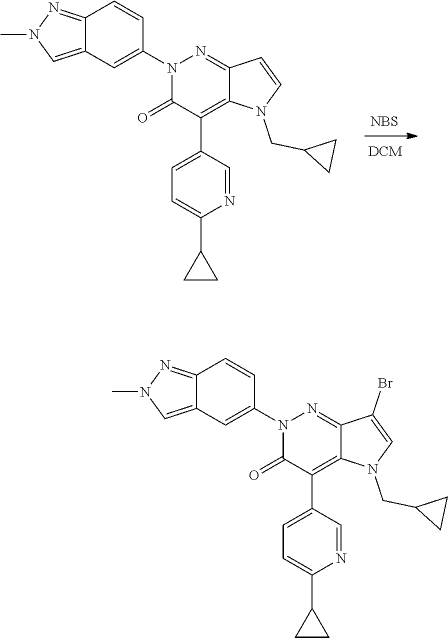

To a solution of 5-(cyclopropylmethyl)-4-(6-cyclopropylpyridin-3-yl)-2-(2-methyl-2H-indazol-5-yl)-2H,3H,5H-pyrrolo[3,2-c]pyridazin-3-one (80 mg, 0.183 mmol, 1.0 eq.) in DCM (3 mL) was added NBS (49 mg, 0.275 mmol, 1.5 eq.), the reaction mixture was stirred at room temperature for 10 min. The reaction was quenched with NaS₂O₃ (sat. aq.) (20 ml) and extracted with DCM (50 mL×3). The combined organic layers were washed with brine (50 mL), dried over anhydrous Na₂SO₄, concentrated under reduced pressure, and the residue was purified by RP-Prep-HPLC to give 7-bromo-5-(cyclopropylmethyl)-4-(6-cyclopropylpyridin-3-yl)-2-(2-methyl-2H-indazol-5-yl)-2H,3H,5H-pyrrolo[3,2-c]pyridazin-3-one.

¹H NMR (400 MHz, DMSO) δ (ppm): 8.39 (s, 1H), 8.38 (s, 1H), 7.88 (s, 1H), 7.81 (s, 1H), 7.68 (dd, J=8.0, 2.0 Hz, 1H), 7.57 (d, J=9.2 Hz, 1H), 7.32 (d, J=8.0 Hz, 1H), 7.27 (dd, J=9.2, 2.0 Hz, 1H), 4.11 (s, 3H), 3.20 (d, J=7.2 Hz, 2H), 2.12-2.06 (m, 1H), 0.92-0.86 (m, 4H), 0.67-0.51 (m, 1H), 0.25-0.19 (m, 2H), 0.10-0.01 (m, 2H). LC-MS (ESI): m/z 515 [M+H]⁺.

### Example 239: Synthesis of 5-(cyclopropylmethyl)-4-(6-cyclopropylpyridin-3-yl)-7-(hydroxymethyl)-2-(2-methyl-2H-indazol-5-yl)-2,5-dihydro-3H-pyrrolo[3,2-c]pyridazin-3-one

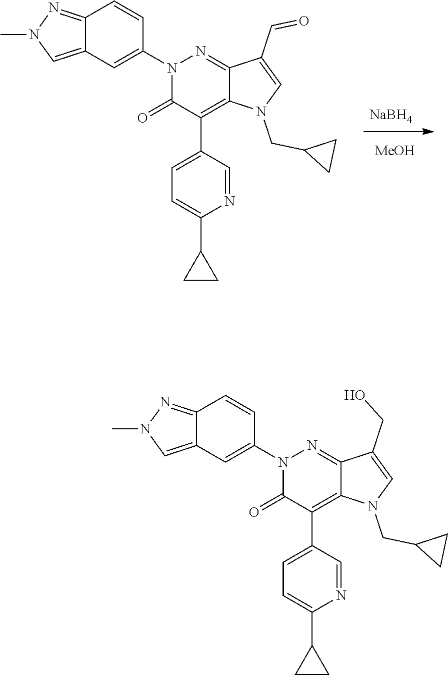

To a solution of 5-(cyclopropylmethyl)-4-(6-cyclopropylpyridin-3-yl)-2-(2-methyl-2H-indazol-5-yl)-3-oxo-3,5-dihydro-2H-pyrrolo[3,2-c]pyridazine-7-carbaldehyde (50 mg, 0.11 mmol, 1.0 eq.) in MeOH (3 mL) was added NaBH₄ (12 mg, 0.32 mmol, 3.0 eq.) at 0° C. The reaction mixture was stirred at 0° C. for 1 hr. After completion, the resulting mixture was poured into NH₄Cl (sat. aq.) (15 mL) and extracted with DCM (10 mL×3). The combined organic layers were washed with brine (20 mL), dried over Na₂SO₄ and concentrated under reduced pressure. The resulting residue was purified by Prep-TLC and RP-Prep-HPLC to give 5-(cyclopropylmethyl)-4-(6-cyclopropylpyridin-3-yl)-7-(hydroxymethyl)-2-(2-methyl-2H-indazol-5-yl)-2,5-dihydro-3H-pyrrolo[3,2-c]pyridazin-3-one (Example 239).

¹H NMR (400 MHz, DMSO-d₆) δ (ppm): 8.45 (d, J=2.0 Hz, 1H), 8.44 (s, 1H), 7.88 (d, J=1.2 Hz, 1H), 7.75 (dd, J=8.0, 2.0 Hz, 1H), 7.65 (d, J=9.2 Hz, 1H), 7.56 (s, 1H), 7.41 (d, J=8.0 Hz, 1H), 7.36 (dd, J=9.2, 2.0 Hz, 1H), 5.01 (s, 1H), 4.50 (s, 2H), 4.20 (s, 3H), 3.29 (d, J=7.2 Hz, 2H), 2.21-2.12 (m, 1H), 1.04-0.94 (m, 4H), 0.73-0.62 (m, 1H), 0.35-0.27 (m, 2H), 0.10-0.03 (m, 2H). LC-MS (ESI): m/z 467 [M+H]⁺.

### Example 240: Synthesis of 5-(cyclopropylmethyl)-4-(6-cyclopropylpyridin-3-yl)-7-(difluoromethyl)-2-(2-methyl-2H-indazol-5-yl)-2,5-dihydro-3H-pyrrolo[3,2-c]pyridazin-3-one

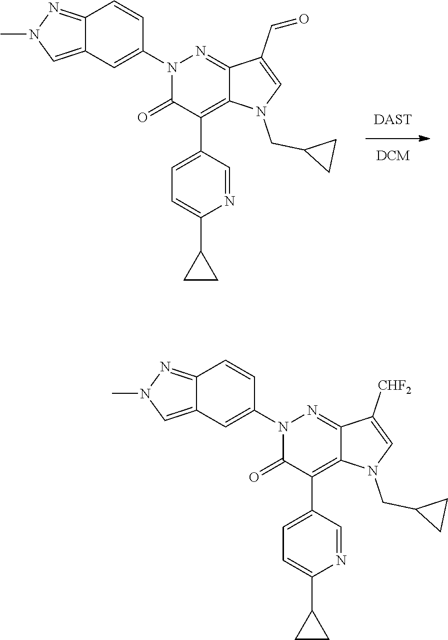

To a solution of 5-(cyclopropylmethyl)-4-(6-cyclopropylpyridin-3-yl)-2-(2-methyl-2H-indazol-5-yl)-3-oxo-2H,3H,5H-pyrrolo[3,2-c]pyridazine-7-carbaldehyde (50 mg, 0.11 mmol, 1.0 eq.) in DCM (5 mL) was added DAST (52 mg, 0.33 mmol, 3.0 eq.) at 0° C. The reaction mixture was stirred at room temperature for 5 hrs. The resulting mixture was poured into ice-cooled NaHCO₃(sat. aq.) (15 mL) and extracted with DCM (10 mL×3). The combined organic layers were washed with brine (10 mL), dried over Na₂SO₄ and concentrated under reduced pressure. The resulting residue was purified by Prep-TLC to give 5-(cyclopropylmethyl)-4-(6-cyclopropylpyridin-3-yl)-7-(hydroxymethyl)-2-(2-methyl-2H-indazol-5-yl)-2,5-dihydro-3H-pyrrolo[3,2-c]pyridazin-3-one.

¹H NMR (400 MHz, DMSO-d₆) δ (ppm): 8.38 (s, 1H), 8.37 (s, 1H), 8.01 (s, 1H), 7.78 (d, J=1.6 Hz, 1H), 7.68 (dd, J=8.0, 2.0 Hz, 1H), 7.55 (d, J=9.2 Hz, 1H), 7.31 (d, J=8.0 Hz, 1H), 7.25 (dd, J=9.2, 2.0 Hz, 1H), 7.02 (t, J_{HF}=54.8 Hz, 1H), 4.10 (s, 3H), 3.25 (d, J=7.2 Hz, 2H), 2.13-2.03 (m, 1H), 0.95-0.81 (m, 4H), 0.62-0.51 (m, 1H), 0.28-0.17 (m, 2H), 0.10-0.01 (m, 2H).

LC-MS (ESI): m/z 487 [M+H]⁺.

### Examples 241 & 242: Synthesis of 5-(cyclopropylmethyl)-4-(6-cyclopropylpyridin-3-yl)-2-(2-methyl-3-(trifluoromethyl)-2H-indazol-5-yl)-2,5-dihydro-3H-pyrrolo[3,2-c]pyridazin-3-one (Example 241) and 5-(cyclopropylmethyl)-4-(6-cyclopropylpyridin-3-yl)-2-(2-methyl-3-(trifluoromethyl)-2H-indazol-5-yl)-7-(trifluoromethyl-2,5-dihydro-3H-pyrrolo[3,2-c]pyridazin-3-one (Example 242)

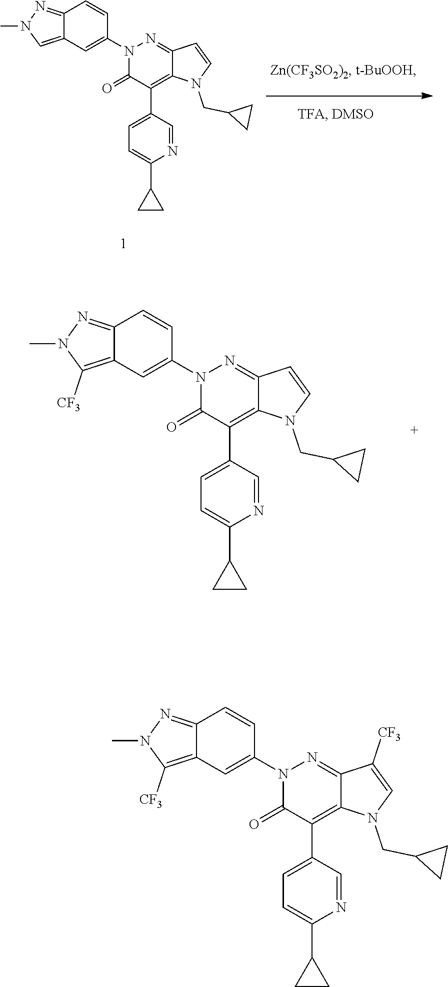 [SMILES: Cn1cc2cc(-n3nc4ccn(CC5CC5)c4c(-c4ccc(C5CC5)nc4)c3=O)ccc2n1 ; Cn1nc2ccc(-n3nc4ccn(CC5CC5)c4c(-c4ccc(C5CC5)nc4)c3=O)cc2c1C(F)(F)F ; Cn1nc2ccc(-n3nc4c(C(F)(F)F)cn(CC5CC5)c4c(-c4ccc(C5CC5)nc4)c3=O)cc2c1C(F)(F)F]

To a solution of 5-(cyclopropylmethyl)-4-(6-cyclopropylpyridin-3-yl)-2-(2-methyl-2H-indazol-5-yl)-2H,3H,5H-pyrrolopyridazin-3-one (60 mg, 0.14 mmol, 1.0 eq.) in DMSO (1 mL) was added DFMS (81 mg, 0.28 mmol, 2.0 eq.), then TFA (10 μL, 0.14 mmol, 1.0 eq.) and then TBHP (52 μL, 0.22 mmol, 1.6 eq.) was added at 0° C. The reaction mixture was stirred at room temperature for 2 hrs. The reaction was quenched with NaHCO₃(sat. aq.) (15 mL) and extracted with EtOAc (10 mL×3). The combined organic layers were washed with brine (20 mL), dried over Na₂SO₄, concentrated under reduced pressure, and the residue was purified by flash column chromatography on silica gel and Prep-TLC to give 5-(cyclopropylmethyl)-4-(6-cyclopropylpyridin-3-yl)-2-(2-methyl-3-(trifluoromethyl)-2H-indazol-5-yl)-2,5-dihydro-3H-pyrrolo[3,2-c]pyridazin-3-one (Example 241) and 5-(cyclopropylmethyl)-4-(6-cyclopropylpyridin-3-yl)-2-(2-methyl-3-(trifluoromethyl)-2H-indazol-5-yl)-7-(trifluoromethyl)-2,5-dihydro-3H-pyrrolo[3,2-c]pyridazin-3-one (Example 242).

Example 241: ¹H NMR (400 MHz, DMSO-d₆) δ (ppm): 8.47 (d, J=1.8 Hz, 1H), 7.96-7.83 (m, 2H), 7.79-7.70 (m, 2H), 7.59 (dd, J=9.2, 1.9 Hz, 1H), 7.41 (d, J=7.9 Hz, 1H), 6.47 (d, J=3.8 Hz, 1H), 4.35 (s, 3H), 3.34 (d, J=7.2 Hz, 2H), 2.20-2.16 (m, 1H), 1.03-0.95 (m, 4H), 0.69-0.51 (m, 1H), 0.32-0.25 (m, 2H), 0.10-0.06 (m, 2H). LC-MS (ESI): m/z 505 [M+H]⁺.

Example 242: ¹H NMR (400 MHz, DMSO-d₆) δ (ppm): 8.50 (d, J=1.7 Hz, 1H), 8.45 (d, J=1.3 Hz, 1H), 7.97-7.89 (m, 2H), 7.81 (dd, J=8.0, 2.2 Hz, 1H), 7.57 (dd, J=9.2, 1.9 Hz, 1H), 7.44 (d, J=8.1 Hz, 1H), 4.36 (s, 3H), 3.39 (d, J=7.1 Hz, 2H), 2.25-2.18 (m, 1H), 1.04-0.96 (m, 4H), 0.71-0.50 (m, 1H), 0.35-0.28 (m, 2H), 0.13-0.07 (m, 2H). LC-MS (ESI): m/z 573 [M+H]⁺.

### Example 245: Synthesis of 5-(cyclopropylmethyl)-4-(6-cyclopropylpyridin-3-yl)-2-(2-methyl-2H-indazol-5-yl)-7-(3,3,3-trifluoroprop-1-yn-1-yl)-2,5-dihydro-3H-pyrrolo[3,2-c]pyridazin-3-one

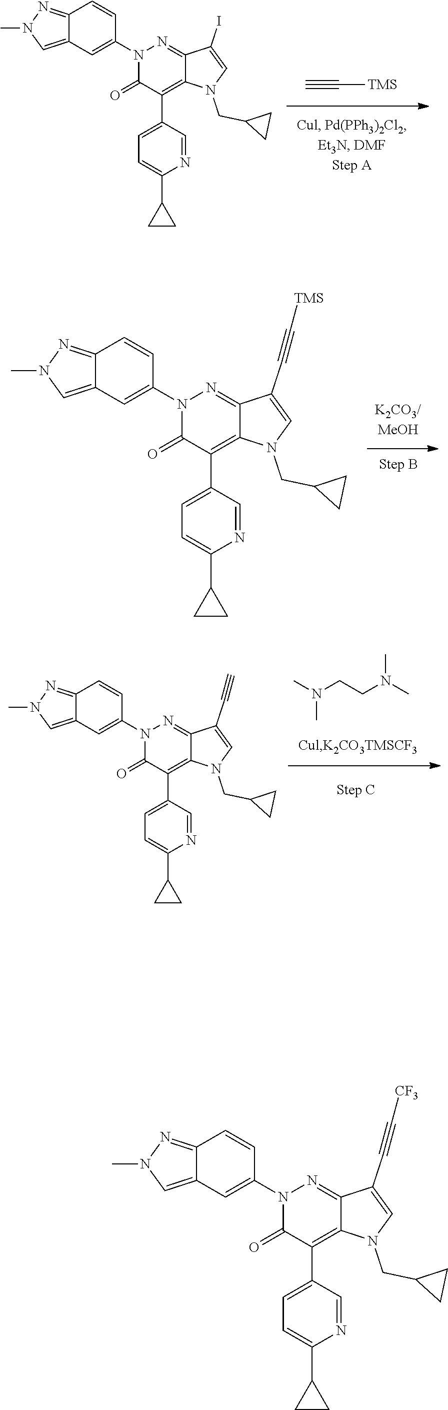

### Step A: 5-(cyclopropylmethyl)-4-(6-cyclopropylpyridin-3-yl)-2-(2-methyl-2H-indazol-5-yl)-7-((trimethylsilyl)ethynyl)-2,5-dihydro-3H-pyrrolo[3,2-c]pyridazin-3-one

A solution of 5-(cyclopropylmethyl)-4-(6-cyclopropylpyridin-3-yl)-7-iodo-2-(2-methyl-2H-indazol-5-yl)-2,5-dihydro-3H-pyrrolo[3,2-c]pyridazin-3-one (200 mg, 0.36 mmol, 1.0 eq.), CuI (4 mg, 0.02 mmol, 0.05 eq.), Pd(PPh₃)₂Cl₂ (13 mg, 0.02 mmol, 0.05 eq.) in DMF (2 mL) and Et₃N (0.2 mL) was degassed with N₂, then ethynyltrimethylsilane (105 mg, 1.0 mmol, 3.0 eq.) was added. The reaction mixture was stirred at 23° C. for 4 hrs. The reaction was quenched by adding NH₄Cl (sat. aq.) (10 mL) and extracted with EtOAc (10 mL×3). The organic layers were combined and washed with brine (20 mL), dried over Na₂SO₄, and concentrated under reduced pressure. The resulting residue was purified by flash column chromatography on silica gel to give 5-(cyclopropylmethyl)-4-(6-cyclopropylpyridin-3-yl)-2-(2-methyl-2H-indazol-5-yl)-7-((trimethylsilyl)ethynyl)-2,5-dihydro-3H-pyrrolo[3,2-c]pyridazin-3-one. (Example 243). ¹H NMR (400 MHz, DMSO-d₆) δ (ppm): 8.47 (d, J=2.0 Hz, 1H), 8.46 (s, 1H), 8.12 (s, 1H), 7.89 (d, J=1.6 Hz, 1H), 7.77 (dd, J=8.0, 2.2 Hz, 1H), 7.66 (d, J=9.1 Hz, 1H), 7.41 (d, J=8.0 Hz, 1H), 7.34 (dd, J=9.1, 2.0 Hz, 1H), 4.21 (s, 3H), 3.30 (d, J=7.2 Hz, 2H), 2.20-2.10 (m, 1H), 1.24-0.96 (m, 4H), 0.75-0.64 (m, 1H), 0.35-0.30 (m, 2H), 0.19 (s, 9H), 0.12-0.08 (m, 2H). LC-MS (ESI): m/z 533 [M+H]⁺.

### Step B: 5-(cyclopropylmethyl)-4-(6-cyclopropylpyridin-3-yl)-7-ethynyl-2-(2-methyl-2H-indazol-5-yl)-2,5-dihydro-3H-pyrrolo[3,2-c]pyridazin-3-one

To a solution of 5-(cyclopropylmethyl)-4-(6-cyclopropylpyridin-3-yl)-2-(2-methyl-2H-indazol-5-yl)-7-((trimethylsilyl)ethynyl)-2,5-dihydro-3H-pyrrolo[3,2-c]pyridazin-3-one (170 mg, 0.3 mmol, 1.0 eq.) in MeOH (3 mL) was added K₂CO₃ (132 mg, 0.9 mmol, 3.0 eq.) and the mixture was stirred at room temperature for 1h. The reaction mixture was filtered and the filter cake was washed with 10 mL of MeOH. The combined organic layers were concentrated in vacuo and purified by flash column chromatography on silica gel to give 5-(cyclopropylmethyl)-4-(6-cyclopropylpyridin-3-yl)-7-ethynyl-2-(2-methyl-2H-indazol-5-yl)-2,5-dihydro-3H-pyrrolo[3,2-c]pyridazin-3-one (Example 244). ¹H NMR (400 MHz, DMSO-d₆) δ (ppm): 8.37 (d, J=1.8 Hz, 1H), 8.34 (s, 1H), 7.99 (s, 1H), 7.80 (d, J=1.3 Hz, 1H), 7.67 (dd, J=8.0, 2.2 Hz, 1H), 7.56 (d, J=9.1 Hz, 1H), 7.31 (d, J=8.0 Hz, 1H), 7.26 (dd, J=9.1, 2.0 Hz, 1H), 4.14 (s, 1H), 4.10 (s, 3H), 3.19 (d, J=7.2 Hz, 2H), 2.13-2.02 (m, 1H), 0.96-0.82 (m, 4H), 0.64-0.53 (m, 1H), 0.30-0.16 (m, 2H), −0.01-0.02 (m, 2H). LC-MS (ESI): m/z 461 [M+H]⁺.

### Step C: 5-(cyclopropylmethyl)-4-(6-cyclopropylpyridin-3-yl)-2-(2-methyl-2H-indazol-5-yl)-7-(3,3,3-trifluoroprop-1-yn-1-yl)-2,5-dihydro-3H-pyrrolo[3,2-c]pyridazin-3-one

To a solution of N¹,N¹,N²,N²-tetramethylethane-1,2-diamine (30 mg, 0.26 mmol, 1.5 eq.) in DMF (2 mL) was added CuI (49 mg, 0.26 mmol, 1.5 eq.) and K₂CO₃ (72 mg, 0.521 mmol, 3.0 eq.). The mixture was stirred for 20 min at room temperature, then TMSCF₃ (247 mg, 1.7 mmol, 10 eq.) was added and stirred for another 15 min under 02. The reaction was then cooled to 0° C., and a mixture of 5-(cyclopropylmethyl)-4-(6-cyclopropylpyridin-3-yl)-7-ethynyl-2-(2-methyl-2H-indazol-5-yl)-2,5-dihydro-3H-pyrrolo[3,2-c]pyridazin-3-one (80 mg, 0.17 mmol, 1.0 eq.) and TMSCF₃ (247 mg, 1.7 mmol, 10 eq.) in DMF (2 mL) was added and stirred for an additional 14 hrs. The reaction was quenched by adding NH₄Cl (sat. aq.) (10 mL) and extracted with EtOAc (10 mL×3). The organic layers were combined and washed with brine (20 mL), dried over Na₂SO₄ and concentrated under reduced pressure. The resulting residue was purified by RP-prep-HPLC to give 5-(cyclopropylmethyl)-4-(6-cyclopropylpyridin-3-yl)-2-(2-methyl-2H-indazol-5-yl)-7-(3,3,3-trifluoroprop-1-yn-1-yl)-2,5-dihydro-3H-pyrrolo[3,2-c]pyridazin-3-one (Example 245). ¹H NMR (400 MHz, DMSO-d₆) δ (ppm): 8.57 (s, 1H), 8.50 (d, J=2.0 Hz, 1H), 8.46 (s, 1H), 7.92 (d, J=1.5 Hz, 1H), 7.80 (dd, J=8.0, 2.2 Hz, 1H), 7.67 (d, J=9.1 Hz, 1H), 7.43 (d, J=8.0 Hz, 1H), 7.36 (dd, J=9.1, 2.0 Hz, 1H), 4.21 (s, 3H), 3.37 (d, J=7.2 Hz, 2H), 2.24-2.15 (m, 1H), 1.02-0.96 (m, 4H), 0.79-0.68 (m, 1H), 0.38-0.33 (m, 2H), 0.15-0.12 (m, 2H). LC-MS (ESI): m/z 529 [M+H]⁺.

### Example 246: Synthesis of 5-(cyclopropylmethyl)-4-(6-cyclopropylpyridin-3-yl)-2-(2-methyl-2H-indazol-5-yl)-7-(trifluoromethyl)-2,5-dihydro-3H-pyrrolo[3,2-c]pyridazin-3-one

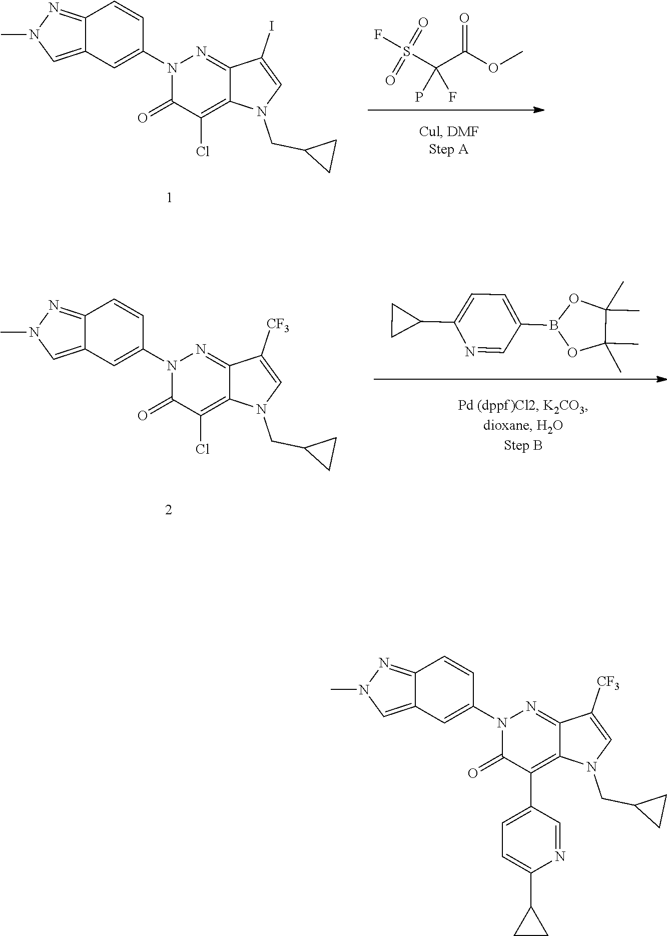

### Step A: 4-chloro-5-(cyclopropylmethyl)-2-(2-methyl-2H-indazol-5-yl)-7-(trifluoromethyl)-2,5-dihydro-3H-pyrrolo[3,2-c]pyridazin-3-one

To a solution of 4-chloro-5-(cyclopropylmethyl)-2-(2-methyl-2H-indazol-5-yl)-2H,3H,5H-pyrrolopyridazin-3-one (213 mg, 0.60 mmol, 1.0 eq.) in DMF (2 mL) was added CuI (57 mg, 0.30 mmol, 0.5 eq.). The reaction mixture was degassed with N₂ three times and methyl 2,2-difluoro-2-(fluorosulfonyl)acetate (307 μL, 2.4 mmol, 4.0 eq.). The reaction vessel was sealed and stirred at 100° C. under microwave irradiation for 1 hour. The reaction mixture was diluted with H₂O (50 mL) and extracted with DCM (10 mL×3). The combined organic layers were washed with brine (20 mL), dried over Na₂SO₄, concentrated under reduced pressure, and the residue was purified by flash chromatography and prep-TLC to afford 4-chloro-5-(cyclopropylmethyl)-2-(2-methyl-2H-indazol-5-yl)-7-(trifluoromethyl)-2H,3H,5H-pyrrolopyridazin-3-one (55 mg, 22%) as a yellow solid. LC-MS (ESI): m/z 422 [M+H]⁺.

### Step B: 5-(cyclopropylmethyl)-4-(6-cyclopropylpyridin-3-yl)-2-(2-methyl-2H-indazol-5-yl)-7-(trifluoromethyl)-2,5-dihydro-3H-pyrrolo[3,2-c]pyridazin-3-one

To a solution of 4-chloro-5-(cyclopropylmethyl)-2-(2-methyl-2H-indazol-5-yl)-7-(trifluoromethyl)-2H,3H,5H-pyrrolopyridazin-3-one (55 mg, 0.13 mmol, 1.0 eq.) and (6-cyclopropylpyridin-3-yl)boronic acid (32 mg, 0.20 mmol, 1.5 eq.) in dioxane (1 mL) was added a solution of K₂CO₃ (36 mg, 0.26 mmol, 2.0 eq.) in H₂O (0.25 mL). The reaction mixture was degassed with N₂ 3 times, Pd(dppf)Cl₂ (5 mg, 0.007 mmol, 0.05 eq.) was added and the mixture was stirred at 100° C. under N₂ for 5 hrs. The reaction mixture was concentrated under reduced pressure, and the residue was purified by flash chromatography and prep-TLC to afford 5-(cyclopropylmethyl)-4-(6-cyclopropylpyridin-3-yl)-2-(2-methyl-2H-indazol-5-yl)-7-(trifluoromethyl)-2H,3H,5H-pyrrolopyridazin-3-one.

¹H NMR (400 MHz, DMSO-d₆) δ (ppm): 8.51 (d, J=2.1 Hz, 1H), 8.46 (s, 1H), 8.42 (d, J=1.2 Hz, 1H), 7.90 (d, J=1.5 Hz, 1H), 7.81 (dd, J=8.0, 2.2 Hz, 1H), 7.67 (d, J=9.1 Hz, 1H), 7.43 (d, J=8.0 Hz, 1H), 7.36 (dd, J=9.1, 2.0 Hz, 1H), 4.21 (s, 3H), 3.39 (d, J=7.1 Hz, 2H), 2.25-2.15 (m, 1H), 1.09-0.94 (m, 4H), 0.77-0.64 (m, 1H), 0.37-0.29 (m, 2H), 0.17-0.09 (m, 2H). LC-MS (ESI): m/z 505 [M+H]⁺.

### Example 247: Preparation of 5-(cyclopropylmethyl)-4-(6-cyclopropylpyridin-3-yl)-7-ethyl-2-(2-methyl-2H-indazol-5-yl)-2,5-dihydro-3H-pyrrolo[3,2-c]pyridazin-3-one

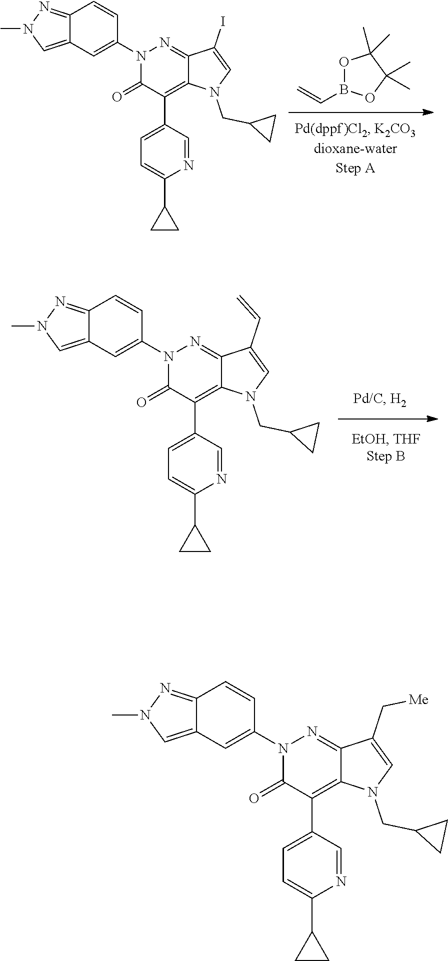

### Step A: 5-(cyclopropylmethyl)-4-(6-cyclopropylpyridin-3-yl)-2-(2-methyl-2H-indazol-5-yl)-7-vinyl-2,5-dihydro-3H-pyrrolo[3,2-c]pyridazin-3-one

To a solution of 5-(cyclopropylmethyl)-4-(6-cyclopropylpyridin-3-yl)-7-iodo-2-(2-methyl-2H-indazol-5-yl)-2H,3H,5H-pyrrolo[3,2-c]pyridazin-3-one (50 mg, 0.089 mmol, 1.0 eq.) in dioxane (4 mL) and H₂O (1.00 mL) was added 2-ethenyl-4,4,5,5-tetramethyl-1,3,2-dioxaborolane (0.018 mL, 0.107 mmol, 1.5 eq.), dipotassium carbonate (37 mg, 0.267 mmol, 3.0 eq.) and Pd(dppf)Cl₂ (13 mg, 0.018 mmol, 0.2 eq.). The reaction was stirred at 90° C. for 4 hrs under N₂. The reaction mixture was concentrated under reduced pressure and purified by column chromatography on silica gel to give 5-(cyclopropylmethyl)-4-(6-cyclopropylpyridin-3-yl)-7-ethenyl-2-(2-methyl-2H-indazol-5-yl)-2H,3H,5H-pyrrolo[3,2-c]pyridazin-3-one (35 mg, 80%) as a yellow solid. LC-MS (ESI): m/z 463 [M+H]⁺.

### Step B: 5-(cyclopropylmethyl)-4-(6-cyclopropylpyridin-3-yl)-7-ethyl-2-(2-methyl-2H-indazol-5-yl)-2,5-dihydro-3H-pyrrolo[3,2-c]pyridazin-3-one

To a solution of 5-(cyclopropylmethyl)-4-(6-cyclopropylpyridin-3-yl)-7-ethenyl-2-(2-methyl-2H-indazol-5-yl)-2H,3H,5H-pyrrolo[3,2-c]pyridazin-3-one (35 mg, 0.076 mmol, 1.0 eq.) in EtOH/THF (3.0 mL, 1:1, v:v), was added Pd/C (5 mg). The resulting mixture was stirred at room temperature for 2 hrs under an H₂ atmosphere. The reaction was filtrated through a short pad of Celite®, the filtrate was concentrated under reduced pressure, and the residue was purified by prep-HPLC to afford 5-(cyclopropylmethyl)-4-(6-cyclopropylpyridin-3-yl)-7-ethyl-2-(2-methyl-2H-indazol-5-yl)-2H,3H,5H-pyrrolo[3,2-c]pyridazin-3-one. ¹H NMR (400 MHz, DMSO-d₆) δ (ppm): 8.45 (d, J=1.8 Hz, 1H), 8.43 (s, 1H), 7.88 (d, J=1.4 Hz, 1H), 7.75 (dd, J=8.0, 2.2 Hz, 1H), 7.64 (d, J=9.2 Hz, 1H), 7.47-7.30 (m, 3H), 4.20 (s, 3H), 3.27 (d, J=7.0 Hz, 2H), 2.55 (q, J=7.5 Hz, 2H), 2.21-2.14 (m, 1H), 1.23 (t, J=7.5 Hz, 3H), 1.00-0.96 (m, 4H), 0.70-0.63 (m, 1H), 0.37-0.21 (m, 2H), 0.13-0.01 (m, 2H). LC-MS (ESI): m/z 465 [M+H]⁺.

### Example 248: Synthesis of 5-(cyclopropylmethyl)-4-(6-cyclopropylpyridin-3-yl)-7-isopropyl-2-(2-methyl-2H-indazol-5-yl)-2,5-dihydro-3H-pyrrolo[3,2-c]pyridazin-3-one

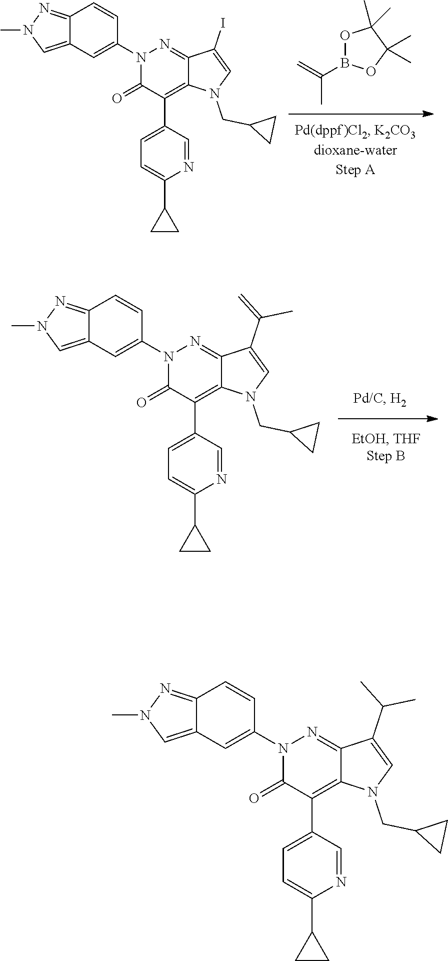 [SMILES: Cn1cc2cc(-n3nc4c(I)cn(CC5CC5)c4c(-c4ccc(C5CC5)nc4)c3=O)ccc2n1 ; C=C(C)B1OC(C)(C)C(C)(C)O1 ; C=C(C)c1cn(CC2CC2)c2c(-c3ccc(C4CC4)nc3)c(=O)n(-c3ccc4nn(C)cc4c3)nc12 ; CC(C)c1cn(CC2CC2)c2c(-c3ccc(C4CC4)nc3)c(=O)n(-c3ccc4nn(C)cc4c3)nc12]

### Step A: 5-(cyclopropylmethyl)-4-(6-cyclopropylpyridin-3-yl)-2-(2-methyl-2H-indazol-5-yl)-7-(prop-1-en-2-yl)-2,5-dihydro-3H-pyrrolo[3,2-c]pyridazin-3-one

To a solution of 5-(cyclopropylmethyl)-4-(6-cyclopropylpyridin-3-yl)-7-iodo-2-(2-methyl-2H-indazol-5-yl)-2H,3H,5H-pyrrolo[3,2-c]pyridazin-3-one (100 mg, 0.178 mmol) in dioxane/H₂O (2.5 mL, 4:1, v:v) was added 4,4,5,5-tetramethyl-2-(prop-1-en-2-yl)-1,3,2-dioxaborolane (0.040 mL, 0.213 mmol), dipotassium carbonate (74 mg, 0.533 mmol) and Pd(dppf)Cl₂ (26 mg, 0.036 mmol). The reaction was stirred at 90° C. for 4 hrs under N₂ atmosphere, then the reaction mixture was concentrated under reduced pressure, and the residue was purified by flash column chromatography on silica gel to give 5-(cyclopropylmethyl)-4-(6-cyclopropylpyridin-3-yl)-2-(2-methyl-2H-indazol-5-yl)-7-(prop-1-en-2-yl)-2H,3H,5H-pyrrolo[3,2-c]pyridazin-3-one (60 mg, 60%) as a yellow solid. LC-MS (ESI): m/z 477 [M+H]⁺.

### Step B: 5-(cyclopropylmethyl)-4-(6-cyclopropylpyridin-3-yl)-7-isopropyl-2-(2-methyl-2H-indazol-5-yl)-2,5-dihydro-3H-pyrrolo[3,2-c]pyridazin-3-one

To a solution of 5-(cyclopropylmethyl)-4-(6-cyclopropylpyridin-3-yl)-2-(2-methyl-2H-indazol-5-yl)-7-(prop-1-en-2-yl)-2H,3H,5H-pyrrolo[3,2-c]pyridazin-3-one (60 mg, 0.126 mmol, 1.0 eq.) in EtOH/THF (4.0 mL, 1:1, v:v), was added Pd/C (20 mg). The resulting mixture was stirred at room temperature for 2 hrs under H₂ atmosphere. The reaction mixture was filtrated through a short pad of Celite®, the filtrate was concentrated under reduced pressure, and the residue was purified by prep-HPLC to afford 5-(cyclopropylmethyl)-4-(6-cyclopropylpyridin-3-yl)-2-(2-methyl-2H-indazol-5-yl)-7-(propan-2-yl)-2H,3H,5H-pyrrolo[3,2-c]pyridazin-3-one. ¹H NMR (400 MHz, DMSO-d₆) δ (ppm): 8.44-8.42 (m, 2H), 7.89 (s, 1H), 7.75 (dd, J=8.0, 2.0 Hz, 1H), 7.65 (d, J=9.2 Hz, 1H), 7.44-7.34 (m, 3H), 4.20 (s, 3H), 3.27 (d, J=7.2 Hz, 2H), 3.01-2.96 (m, 1H), 2.22-2.13 (m, 1H), 1.28 (d, J=6.8 Hz, 6H), 1.03-0.95 (m, 4H), 0.70-0.58 (m, 1H), 0.30-0.21 (m, 2H), 0.10-0.04 (m, 2H). LC-MS (ESI): m/z 479 [M+H]⁺.

### Example 249 & 250: Synthesis of (R)-5-(cyclopropylmethyl)-4-(6-cyclopropylpyridin-3-yl)-7-(1-hydroxyethyl)-2-(2-methyl-2H-indazol-5-yl)-2,5-dihydro-3H-pyrrolo[3,2-c]pyridazin-3-one (Example 249) and (S)-5-(cyclopropylmethyl)-4-(6-cyclopropylpyridin-3-yl)-7-(1-hydroxyethyl)-2-(2-methyl-2H-indazol-5-yl)-2,5-dihydro-3H-pyrrolo[3,2-c]pyridazin-3-one (Example 250)

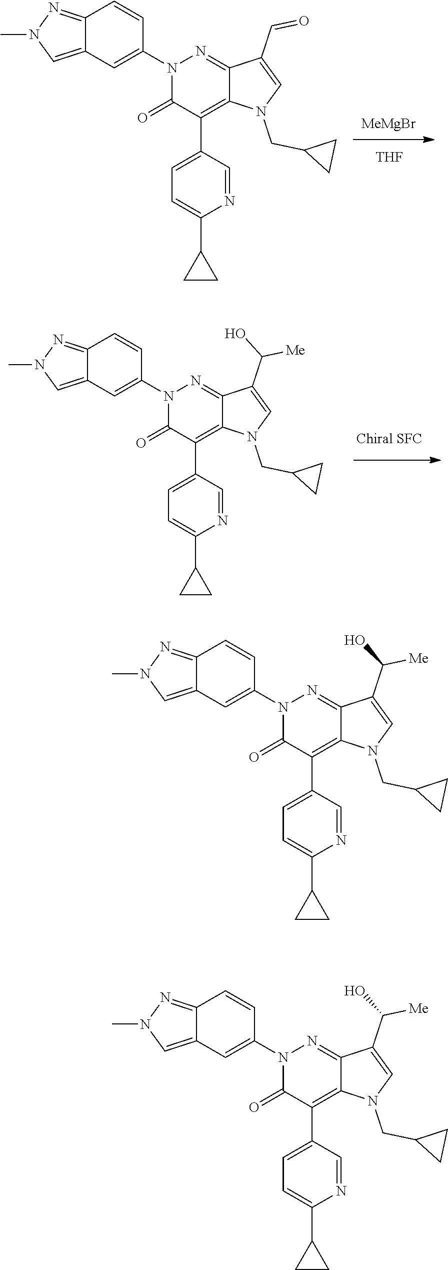 [SMILES: "MeMgBr" ; Cn1cc2cc(-n3nc4c(C=O)cn(CC5CC5)c4c(-c4ccc(C5CC5)nc4)c3=O)ccc2n1 ; "THF" ; CC(O)c1cn(CC2CC2)c2c(-c3ccc(C4CC4)nc3)c(=O)n(-c3ccc4nn(C)cc4c3)nc12 ; C[C@H](O)c1cn(CC2CC2)c2c(-c3ccc(C4CC4)nc3)c(=O)n(-c3ccc4nn(C)cc4c3)nc12 ; C[C@@H](O)c1cn(CC2CC2)c2c(-c3ccc(C4CC4)nc3)c(=O)n(-c3ccc4nn(C)cc4c3)nc12]

To a solution of 5-(cyclopropylmethyl)-4-(6-cyclopropylpyridin-3-yl)-2-(2-methyl-2H-indazol-5-yl)-3-oxo-3,5-dihydro-2H-pyrrolo[3,2-c]pyridazine-7-carbaldehyde (60 mg, 0.13 mmol, 1.0 eq.) in THE (2 mL) was added MeMgBr (0.32 mL, 0.32 mmol, 2.5 eq., 1M in THF) drop-wise at 0° C. The reaction mixture was stirred at 0° C. for 1 hr. After completion, the reaction was quenched with NH₄Cl (sat. aq.) (20 mL), the aqueous layer was extracted by DCM (20 mL×3), the combined organic layers were washed with brine (15 mL), dried over anhydrous Na₂SO₄ and concentrated under reduced pressure. The resulting residue was purified by column chromatography and chiral SFC to give the following pair of enantiomers:

(R)-5-(cyclopropylmethyl)-4-(6-cyclopropylpyridin-3-yl)-7-(1-hydroxyethyl)-2-(2-methyl-2H-indazol-5-yl)-2,5-dihydro-3H-pyrrolo[3,2-c]pyridazin-3-one (Example 249). ¹H NMR (400 MHz, DMSO-d₆) δ (ppm): 8.46 (d, J=1.9 Hz, 1H), 8.43 (s, 1H), 7.89 (d, J=1.4 Hz, 1H), 7.76 (dd, J=8.0, 2.2 Hz, 1H), 7.65 (d, J=9.1 Hz, 1H), 7.49 (s, 1H), 7.40 (d, J=8.0 Hz, 1H), 7.38 (dd, J=9.1, 2.0 Hz, 1H), 5.13 (s, 1H), 4.94-4.80 (m, 1H), 4.20 (s, 3H), 3.29 (d, J=7.1 Hz, 2H), 2.23-2.15 (m, 1H), 1.47 (d, J=6.5 Hz, 3H), 1.00-0.94 (m, 4H), 0.75-0.64 (m, 1H), 0.33-0.25 (m, 2H), 0.11-0.05 (m, 2H). LC-MS (ESI): m/z 481 [M+H]⁺.

(S)-5-(cyclopropylmethyl)-4-(6-cyclopropylpyridin-3-yl)-7-(1-hydroxyethyl)-2-(2-methyl-2H-indazol-5-yl)-2,5-dihydro-3H-pyrrolo[3,2-c]pyridazin-3-one (Example 250). ¹H NMR (400 MHz, DMSO-d₆) δ (ppm): 8.45 (d, J=1.9 Hz, 1H), 8.44 (s, 1H), 7.89 (d, J=1.4 Hz, 1H), 7.75 (dd, J=8.0, 2.2 Hz, 1H), 7.65 (d, J=9.1 Hz, 1H), 7.48 (s, 1H), 7.40 (d, J=8.0 Hz, 1H), 7.37 (dd, J=9.1, 2.0 Hz, 1H), 5.13 (s, 1H), 4.94-4.80 (m, 1H), 4.20 (s, 3H), 3.29 (d, J=7.1 Hz, 2H), 2.23-2.14 (m, 1H), 1.46 (d, J=6.5 Hz, 3H), 1.00-0.94 (m, 4H), 0.72-0.63 (m, 1H), 0.32-0.25 (m, 2H), 0.10-0.04 (m, 2H). LC-MS (ESI): m/z 481 [M+H]⁺.

### Example 251: Synthesis of 6-chloro-5-(cyclopropylmethyl)-4-(6-cyclopropylpyridin-3-yl)-2-(2-methyl-2H-indazol-5-yl)-3-oxo-3,5-dihydro-2H-pyrrolo[3,2-c]pyridazine-7-carbonitrile

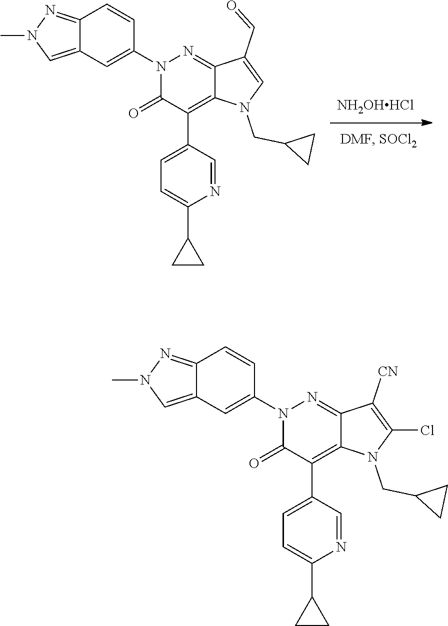

To a solution of 5-(cyclopropylmethyl)-4-(6-cyclopropylpyridin-3-yl)-2-(2-methyl-2H-indazol-5-yl)-3-oxo-3,5-dihydro-2H-pyrrolo[3,2-c]pyridazine-7-carbaldehyde (200 mg, 0.43 mmol, 1.0 eq.) in DMF (4 mL) was added NH₂OH.HCl (44 mg, 0.64 mmol, 1.5 eq.). The reaction mixture was stirred at 40° C. for 2 hrs then cooled to 0° C. and SOCl₂ (153 mg, 1.29 mmol, 3.0 eq.) was added. The resulting mixture was stirred at room temperature for 15 hrs. After completion, the reaction was quenched with NaHCO₃(sat. aq.) (15 mL) and extracted with EtOAc (20 mL×3). The combined organic layers were washed with brine (20 mL), dried over Na₂SO₄ and concentrated under reduced pressure. The resulting residue was purified by Prep-TLC and RP-Prep-HPLC to give 6-chloro-5-(cyclopropylmethyl)-4-(6-cyclopropylpyridin-3-yl)-2-(2-methyl-2H-indazol-5-yl)-3-oxo-3,5-dihydro-2H-pyrrolo[3,2-c]pyridazine-7-carbonitrile. ¹H NMR (400 MHz, DMSO-d₆) δ (ppm): 8.73 (s, 1H), 8.50 (d, J=2.0 Hz, 1H), 7.86 (d, J=2.0 Hz, 1H), 7.80 (dd, J=8.0, 2.0 Hz, 1H), 7.73 (d, J=9.2 Hz, 1H), 7.49 (dd, J=9.2, 2.0 Hz, 1H), 7.44 (d, J=8.0 Hz, 1H), 4.18 (s, 3H), 3.39 (d, J=7.2 Hz, 2H), 2.25-2.13 (m, 1H), 1.05-0.92 (m, 4H), 0.78-0.66 (m, 1H), 0.44-0.30 (m, 2H), 0.17-0.12 (m, 2H). LC-MS (ESI): m/z 496 [M+H]⁺.

### Example 252 & 253: Synthesis of (S)-5-(cyclopropylmethyl)-4-(6-(2,2-difluorocyclopropyl)pyridin-3-yl)-2-(2-methyl-2H-indazol-5-yl)-3-oxo-3,5-dihydro-2H-pyrrolo[3,2-c]pyridazine-7-carbonitrile (Example 252) and (R)-5-(cyclopropylmethyl)-4-(6-(2,2-difluorocyclopropyl)pyridin-3-yl)-2-(2-methyl-2H-indazol-5-yl)-3-oxo-3,5-dihydro-2H-pyrrolo[3,2-c]pyridazine-7-carbonitrile (Example 253)

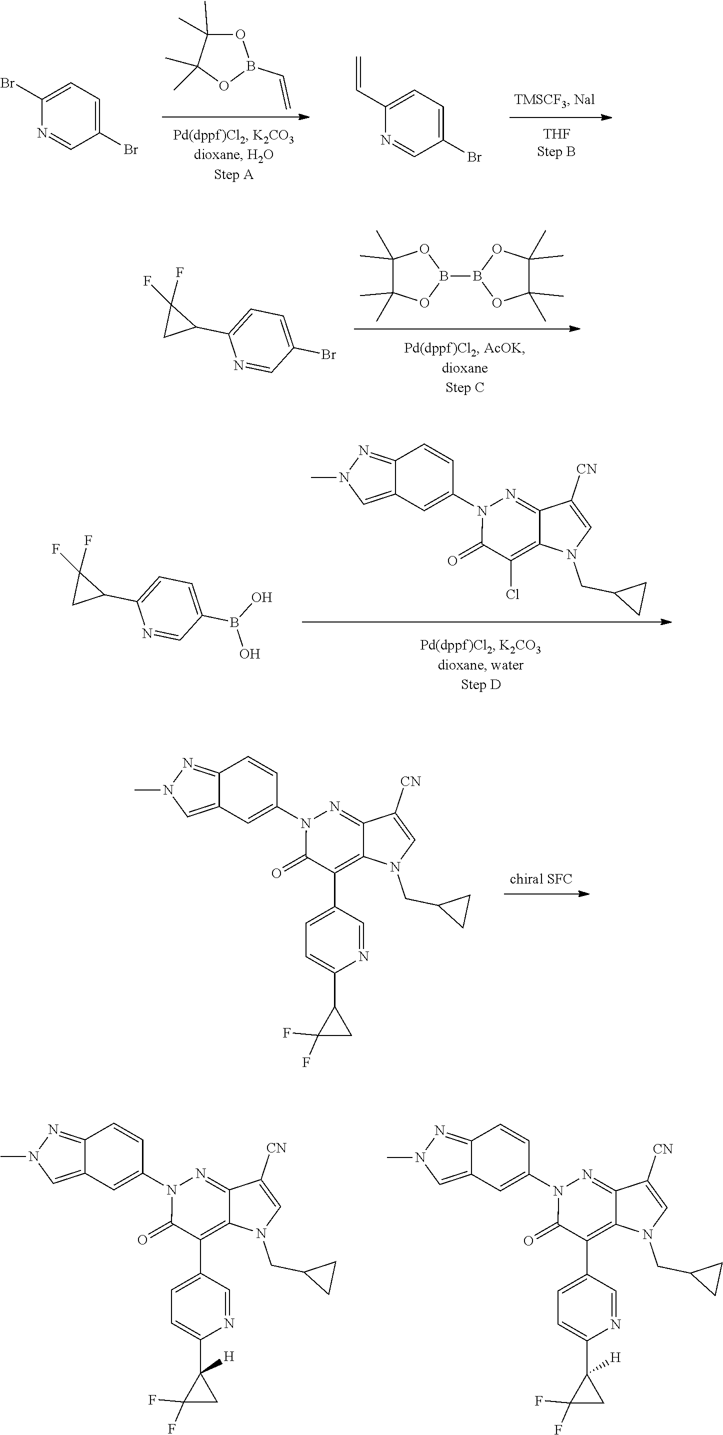

### Step A: 5-bromo-2-ethenylpyridine

To a solution of 2,5-dibromopyridine (1 g, 4.22 mmol, 1.0 eq.) in dioxane (6 mL) was added disodium carbonate (0.89 g, 8.44 mmol, 2.0 eq.), Pd(PPh₃)₄ (0.49 g, 0.42 mmol, 0.1 eq.), pinacol vinylboronate (0.71 g, 4.64 mmol, 1.1 eq.) and H₂O (1.5 mL). The reaction mixture was stirred at 100° C. under N₂ atmosphere for 8 hrs. After completion, the reaction mixture was diluted with H₂O (50 mL) and extracted with EtOAc (50 mL×3). The combined organic layers was dried over Na₂SO₄, filtered, concentrated under reduced pressure, and the resulting residue was purified by flash column chromatography on silica gel to give 5-bromo-2-ethenylpyridine (700 mg, 58%) as a colorless oil. LC-MS (ESI): m/z 184 [M+H]⁺

### Step B: 5-bromo-2-(2,2-difluorocyclopropyl)pyridine

To a solution of 5-bromo-2-ethenylpyridine (250 mg, 1.36 mmol, 1.0 eq.) in THE (5 mL) was added trimethyl(trifluoromethyl)silane (0.6 mL, 4.07 mmol, 3.0 eq.) and NaI (203 mg, 1.36 mmol, 1.0 eq.). The reaction mixture was stirred at 60° C. for 5 hrs. The reaction was quenched with water (30 mL) and extracted with EtOAc (40 mL×3). The combined organic layers was dried over Na₂SO₄, filtered, concentrated under reduced pressure, and the residue was purified by RP-Prep-HPLC to give 5-bromo-2-(2,2-difluorocyclopropyl)pyridine (50 mg, 15%) as a colorless oil. LC-MS (ESI): m/z 234 [M+H]⁺

### Step C: [6-(2,2-difluorocyclopropyl)pyridin-3-yl]boronic Acid

A mixture of 5-bromo-2-(2,2-difluorocyclopropyl)pyridine (30 mg, 0.13 mmol, 1.0 eq.), potassium acetate (56 mg, 0.57 mmol, 4.5 eq.), 4,4,5,5-tetramethyl-2-(tetramethyl-1,3,2-dioxaborolan-2-yl)-1,3,2-dioxaborolane (42 mg, 0.16 mmol, 1.3 eq.) and Pd(dppf)Cl₂ (9 mg, 0.013 mmol, 0.1 eq.) in dioxane (3 mL) was stirred at 100° C. under N₂ atmosphere for 8 hrs. The reaction mixture was used for next step directly without further purification. LC-MS (ESI): m/z 200 [M+H]⁺

### Step D: (S)-5-(cyclopropylmethyl)-4-(6-(2,2-difluorocyclopropyl)pyridin-3-yl)-2-(2-methyl-2H-indazol-5-yl)-3-oxo-3,5-dihydro-2H-pyrrolo[3,2-c]pyridazine-7-carbonitrile and (R)-5-(cyclopropylmethyl)-4-(6-(2,2-difluorocyclopropyl)pyridin-3-yl)-2-(2-methyl-2H-indazol-5-yl)-3-oxo-3,5-dihydro-2H-pyrrolo[3,2-c]pyridazine-7-carbonitrile

To a solution of 4-chloro-5-(cyclopropylmethyl)-2-(2-methyl-2H-indazol-5-yl)-3-oxo-2H,3H,5H-pyrrolo[3,2-c]pyridazine-7-carbonitrile (60 mg, 0.15 mmol, 1.0 eq.) in dioxane (4 mL) was added [6-(2,2-difluorocyclopropyl)pyridin-3-yl]boronic acid (44 mg, 0.22 mmol, 1.4 eq.), dipotassium carbonate (54 mg, 0.39 mmol, 2.5 eq.), Pd(dppf)Cl₂ (11 mg, 0.01 mmol, 0.1 eq.) and water (1 mL). The reaction mixture was stirred at 100° C. N₂ atmosphere for 8 hrs. After completion, the reaction mixture was diluted with water (20 mL) and extracted with EtOAc (20 mL×3). The combined organic layers were washed with brine (20 mL), dried over Na₂SO₄, concentrated under reduced pressure, and the residue was purified by Prep-TLC and chiral SFC to give the pair of enantiomers:

Example 252: (S)-5-(cyclopropylmethyl)-4-(6-(2,2-difluorocyclopropyl)pyridin-3-yl)-2-(2-methyl-2H-indazol-5-yl)-3-oxo-3,5-dihydro-2H-pyrrolo[3,2-c]pyridazine-7-carbonitrile-¹H NMR (400 MHz, DMSO-d₆) δ (ppm): 8.72 (s, 1H), 8.64 (s, 1H), 8.48 (s, 1H), 7.96 (d, J=8.0 Hz, 2H), 7.67 (d, J=9.1 Hz, 1H), 7.60 (d, J=8.1 Hz, 1H), 7.42-7.35 (m, 1H), 4.21 (s, 3H), 3.39-3.35 (m, 2H), 3.29-3.25 (m, 1H), 2.41-2.31 (m, 1H), 2.14-1.99 (m, 1H), 0.71-0.54 (m, 1H), 0.41-0.34 (m, 2H), 0.19-0.06 (m, 2H). LC-MS (ESI): m/z 498 [M+H]⁺

Example 253: (R)-5-(cyclopropylmethyl)-4-(6-(2,2-difluorocyclopropyl)pyridin-3-yl)-2-(2-methyl-2H-indazol-5-yl)-3-oxo-3,5-dihydro-2H-pyrrolo[3,2-c]pyridazine-7-carbonitrile-¹H NMR (400 MHz, DMSO-d₆) δ (ppm): 8.73 (s, 1H), 8.64 (s, 1H), 8.47 (s, 1H), 7.96 (d, J=8.0 Hz, 2H), 7.68 (d, J=9.1 Hz, 1H), 7.60 (d, J=8.1 Hz, 1H), 7.42-7.35 (m, 1H), 4.21 (s, 3H), 3.39-3.35 (m, 2H), 3.28-3.25 (m, 1H), 2.40-2.30 (m, 1H), 2.13-1.99 (m, 1H), 0.71-0.54 (m, 1H), 0.42-0.34 (m, 2H), 0.18-0.07 (m, 2H). LC-MS (ESI): m/z 498 [M+H]⁺

### Example 254: Synthesis of 5-(cyclopropylmethyl)-2-(2-methyl-2H-indazol-5-yl)-4-[6-(1-methylcyclopropyl)pyridin-3-yl]-3-oxo-2H,3H,5H-pyrrolo[3,2-c]pyridazine-7-carbonitrile

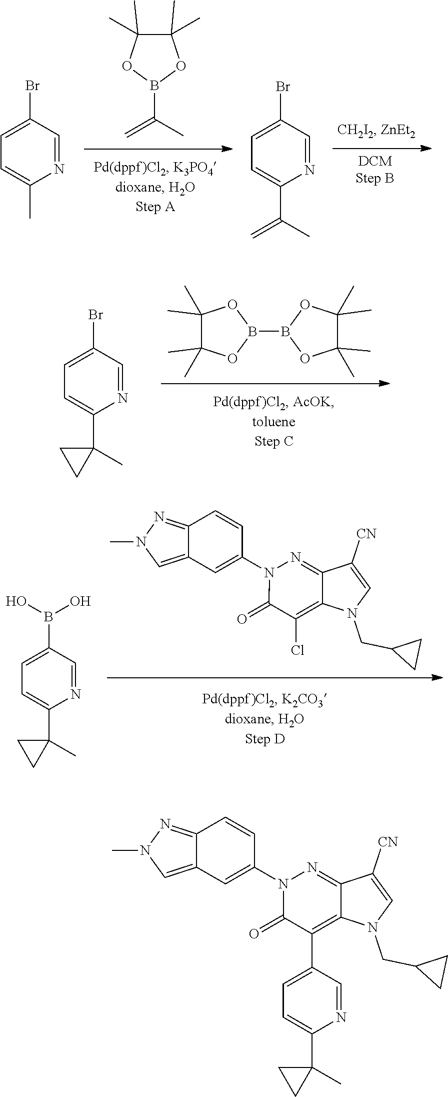

### Step A: 5-bromo-2-(prop-1-en-2-yl)pyridine

To a solution of 5-bromo-2-iodopyridine (2 g, 7.04 mmol, 1.0 eq.), 4,4,5,5-tetramethyl-2-(prop-1-en-2-yl)-1,3,2-dioxaborolane (1.3 g, 7.75 mmol, 1.1 eq.) and K₃PO₄ (3 g, 14.09 mmol, 2.0 eq.) in dioxane (16 mL) and H₂O (4 mL) was added Pd(dppf)Cl₂ (258 mg, 0.352 mmol, 0.05 eq.). The reaction mixture was degassed with N₂ and stirred under N₂ atmosphere at 100° C. for 4 hrs. After completion, the reaction mixture was diluted H₂O (40 mL) and extracted with EtOAc (40 mL×3). The combined organic layers were washed with brine (20 mL), dried over Na₂SO₄ and evaporated to dryness. The resulting residue was purified by flash column chromatography on silica gel to give 5-bromo-2-(prop-1-en-2-yl)pyridine(960 mg, 69%) as pale yellow oil. LC-MS (ESI): m/z 198 [M+H]⁺

### Step B: 5-bromo-2-(1-methylcyclopropyl)pyridine

To a solution of TFA (2.1 mL, 29.03 mmol, 11.5 eq.) in DCM (3 mL) was added a solution of diiodomethane (2.3 mL, 29.03 mmol, 11.5 eq.) in DCM (3 mL), then the mixture was stirred at 0° C. for 0.5 h. A solution of diethylzinc (29 mL, 29.03 mmol, 11.5 eq.) in DCM (3 mL) was then added at 0° C. and the mixture was stirred at 0° C. for 0.5 h. To the mixture was added a solution of 5-bromo-2-(prop-1-en-2-yl)pyridine (500 mg, 2.52 mmol, 1.0 eq.) in DCM (3 mL) at 0° C., and the mixture was stirred at 20° C. for 8 hrs. The mixture was quenched with water (50 mL) and extracted with DCM (50 mL×3). The combined organic phase was dried over Na₂SO₄, filtered, concentrated under reduced pressure, and the residue was purified by Prep-HPLC to give of 5-bromo-2-(1-methylcyclopropyl)pyridine (110 mg, 20%) as pale yellow oil. LC-MS (ESI): m/z 212 [M+H]⁺

### Step C: [6-(1-methylcyclopropyl)pyridin-3-yl]boronic Acid

To a solution of 5-bromo-2-(1-methylcyclopropyl)pyridine (50 mg, 0.23 mmol, 1.0 eq.) in dioxane (3 mL) was added Pd(dppf)Cl₂ (17 mg, 0.02 mmol, 0.1 eq.), 4,4,5,5-tetramethyl-2-(tetramethyl-1,3,2-dioxaborolan-2-yl)-1,3,2-dioxaborolane (78 mg, 0.30 mmol, 1.3 eq.) and potassium acetate (104 mg, 1.06 mmol, 4.5 eq.), and the mixture was stirred at 100° C. for 4 hrs. The mixture was concentrated under reduced pressure, which was used in next step directly without further purification. LC-MS (ESI): m/z 178 [M+H]⁺

### Step D: 5-(cyclopropylmethyl)-2-(2-methyl-2H-indazol-5-yl)-4-[6-(1-methylcyclopropyl)pyridin-3-yl]-3-oxo-2H,3H,5H-pyrrolo[3,2-c]pyridazine-7-carbonitrile

To a solution of 4-chloro-5-(cyclopropylmethyl)-2-(2-methyl-2H-indazol-5-yl)-3-oxo-2H,3H,5H-pyrrolo[3,2-c]pyridazine-7-carbonitrile (60 mg, 0.15 mmol, 1.0 eq.) in dioxane/H₂O (4 mL, 3:1, v:v) was added [6-(1-methylcyclopropyl)pyridin-3-yl]boronic acid (39 mg, 0.22 mmol, 1.4 eq.), dipotassium carbonate (54 mg, 0.39 mmol, 2.5 eq.), Pd(dppf)Cl₂ (11 mg, 0.01 mmol, 0.1 eq.), and the resulting mixture was stirred at 100° C. for 8 hrs. The mixture was quenched by adding H₂O (10 mL) and extracted with EtOAc (10 mL×3). The combined organic phase was dried over Na₂SO₄, filtered and concentrated under reduced pressure. The resulting residue was purified by Prep-TLC to give 5-(cyclopropylmethyl)-2-(2-methyl-2H-indazol-5-yl)-4-[6-(1-methylcyclopropyl)pyridin-3-yl]-3-oxo-2H,3H,5H-pyrrolo[3,2-c]pyridazine-7-carbonitrile. ¹H NMR (400 MHz, DMSO-d₆): δ (ppm) 8.73 (s, 1H), 8.53 (d, J=1.8 Hz, 1H), 8.47 (s, 1H), 7.94 (s, 1H), 7.85 (dd, J=8.2, 2.2 Hz, 1H), 7.67 (d, J=9.2 Hz, 1H), 7.48 (d, J=8.3 Hz, 1H), 7.38 (dd, J=9.2, 1.9 Hz, 1H), 4.21 (s, 3H), 3.39 (d, J=7.1 Hz, 2H), 1.52 (s, 3H), 1.24-1.22 (m, 2H), 0.90-0.81 (m, 2H), 0.76-0.68 (m, 1H), 0.38-0.34 (m, 2H), 0.20-0.13 (m, 2H). LC-MS (ESI): m/z 476 [M+H]⁺

### Example 255: Synthesis of 5-(cyclopropylmethyl)-7-(hydroxymethyl)-2-(2-methyl-2H-indazol-5-yl)-4-(6-methylpyridin-3-yl)-2,5-dihydro-3H-pyrrolo[3,2-c]pyridazin-3-one

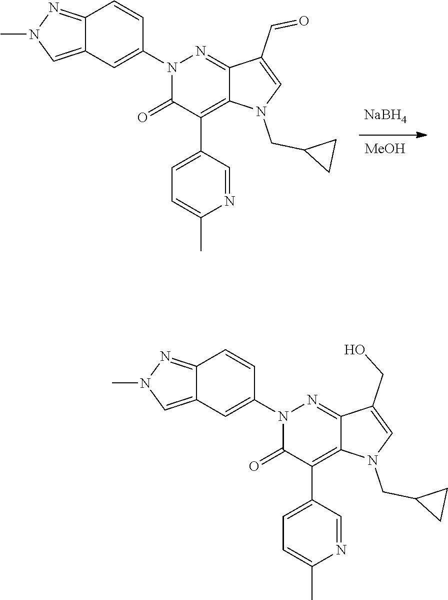

To a solution of 5-(cyclopropylmethyl)-2-(2-methyl-2H-indazol-5-yl)-4-(6-methylpyridin-3-yl)-3-oxo-3,5-dihydro-2H-pyrrolo[3,2-c]pyridazine-7-carbaldehyde (50 mg, 0.11 mmol, 1.0 eq.) in MeOH (3 mL) was added NaBH₄ (12 mg, 0.33 mmol, 3.0 eq.) at 0° C. The reaction mixture was kept stirring at 0° C. for additional 1 hr. The reaction was quenched with ice-cooled NH₄Cl (sat. aq.) (5 mL), extracted with DCM (10 mL×3), the organic layers were combined and washed with brine (20 mL), dried over anhydrous Na₂SO₄, filtered, concentrated under reduced pressure, and the residue was purified by prep-TLC and prep-HPLC to give 5-(cyclopropylmethyl)-7-(hydroxymethyl)-2-(2-methyl-2H-indazol-5-yl)-4-(6-methylpyridin-3-yl)-2,5-dihydro-3H-pyrrolo[3,2-c]pyridazin-3-one. ¹H NMR (400 MHz, DMSO-d₆) δ (ppm): 8.45 (d, J=2.0 Hz, 1H), 8.44 (s, 1H), 7.88 (d, J=1.2 Hz, 1H), 7.75 (dd, J=8.0, 2.0 Hz, 1H), 7.65 (d, J=9.2 Hz, 1H), 7.56 (s, 1H), 7.41 (d, J=8.0 Hz, 1H), 7.36 (dd, J=9.2, 2.0 Hz, 1H), 5.01 (t, J=5.2 Hz, 1H), 4.50 (d, J=5.2 Hz, 2H), 4.20 (s, 3H), 3.29 (d, J=7.2 Hz, 2H), 2.54 (s, 3H), 0.73-0.62 (m, 1H), 0.35-0.27 (m, 2H), 0.10-0.03 (m, 2H). LC-MS (ESI): m/z 441 [M+H]⁺.

### Examples 256 & 257: Preparation of 5-(cyclopropylmethyl)-4-(6-(1-hydroxycyclopropyl)pyridin-3-yl)-2-(2-methyl-2H-indazol-5-yl)-3-oxo-3,5-dihydro-2H-pyrrolo[3,2-c]pyridazine-7-carbonitrile and 5-(cyclopropylmethyl)-4-(6-(3-fluoroprop-1-en-2-yl)pyridin-3-yl)-2-(2-methyl-2H-indazol-5-yl)-3-oxo-3,5-dihydro-2H-pyrrolo[3,2-c]pyridazine-7-carbonitrile

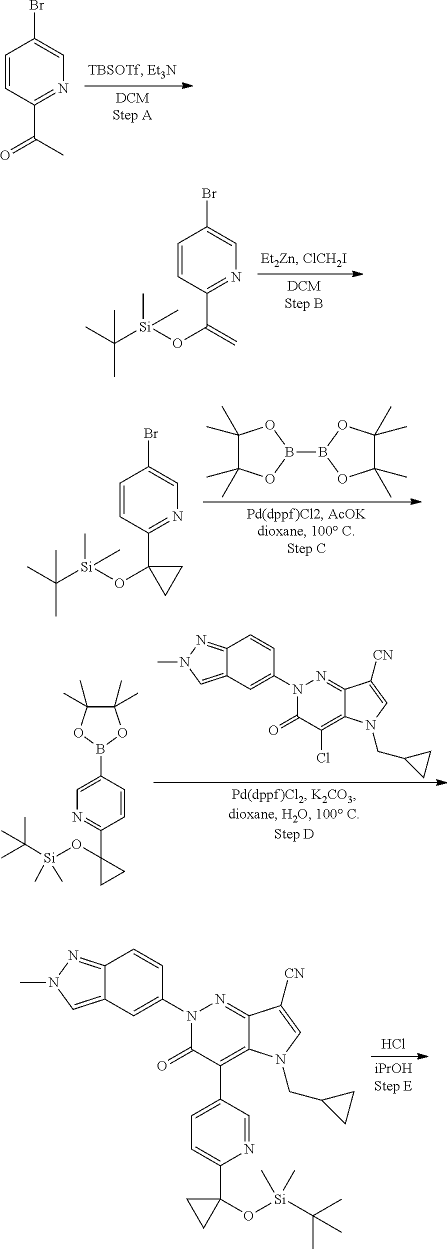

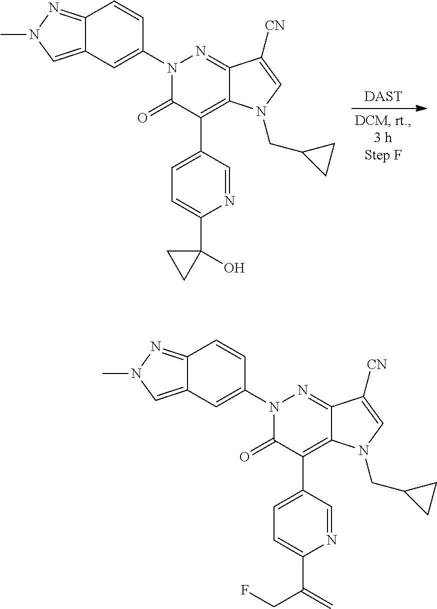

### Step A: 5-bromo-2-(1-((tert-butyldimethylsilyl)oxy)vinyl)pyridine

To the solution of 1-(5-bromopyridin-2-yl)ethan-1-one (1.3 g, 6.499 mmol, 1.0 eq.) and TEA (2.7 mL, 19.497 mmol, 3.0 eq.) in DCM (20 mL) was added TBSOTf (2.57 g, 9.749 mmol, 1.5 eq.) at 0° C. and the mixture stirred at room temperature for 3 hrs. The mixture was quenched with NaHCO₃(sat. aq.) (5 mL) and extracted with DCM (10 mL×3). The organic layers were combined and washed with brine (5 mL), dried over anhydrous Na₂SO₄, filtered and concentrated under reduced pressure. The resulting residue was purified by flash column chromatography to give 5-bromo-2-{1-ethenyl}pyridine (1.9 g, 93%) as a colorless oil. LC-MS (ESI): m/z 314,316 [M+H]⁺

### Step B: 5-bromo-2-(1-((tert-butyldimethylsilyl)oxy)cyclopropyl)pyridine

To a solution of diethylzinc (18.1 mL, 18.136 mmol, 3.0 eq.) in DCM (30 mL) at 0° C. was added chloroiodomethane (2.6 mL, 36.271 mmol, 6.0 eq.) dissolved in DCM (3 mL) dropwise. The reaction mixture was stirred at 0° C. for 15 min. A solution of 5-bromo-2-(1-((tert-butyldimethylsilyl)oxy)vinyl)pyridine (1.9 g, 6.045 mmol, 1.0 eq.) in DCM (5 mL) was added. The reaction mixture was stirred at 0° C. for 2 hrs. The reaction was quenched by adding ice-cooled NH₄Cl (sat. aq.) (20 ml), extracted with DCM (30 mL×3), all the organic layers were combined and washed with brine (30 mL), dried over Na₂SO₄ and concentrated under reduced pressure. The residue was purified by flash column chromatography to give 5-bromo-2-(1-((tert-butyldimethylsilyl)oxy)cyclopropyl)pyridine (1 g, 3.046 mmol, 50%) as colorless oil. LC-MS (ESI): m/z 328, 330 [M+H]⁺

### Step C: 2-(1-((tert-butyldimethylsilyl)oxy)cyclopropyl)-5-(4,4,5,5-tetramethyl-1,3,2-dioxaborolan-2-yl)pyridine

To a solution of 5-bromo-2-(1-((tert-butyldimethylsilyl)oxy)cyclopropyl)pyridine (500 mg, 1.523 mmol, 1.0 eq.), Pin₂B₂ (406 mg, 1.599 mmol, 1.05 eq.), AcOK (298 mg, 3.046 mmol, 2.0 eq.) in dioxane (6 mL) was added Pd(dppf)Cl₂ (56 mg, 0.076 mmol, 0.05 eq.). The resulting mixture was kept stirring at 100° C. under N₂ atmosphere for 3 hrs. After cooling, the mixture was concentrated under reduced pressure and purified by flash column chromatography on silica gel to give 2-(1-((tert-butyldimethylsilyl)oxy)cyclopropyl)-5-(4,4,5,5-tetramethyl-1,3,2-dioxaborolan-2-yl)pyridine (450 mg, 79%) as a colorless oil. LC-MS (ESI): m/z 294 [M+H]⁺

### Step D: 4-(6-(1-((tert-butyldimethylsilyl)oxy)cyclopropyl)pyridin-3-yl)-5-(cyclopropylmethyl)-2-(2-methyl-2H-indazol-5-yl)-3-oxo-3,5-dihydro-2H-pyrrolo[3,2-c]pyridazine-7-carbonitrile

To a solution of 4-chloro-5-(cyclopropylmethyl)-2-(2-methyl-2H-indazol-5-yl)-3-oxo-2H,3H,5H-pyrrolopyridazine-7-carbonitrile (100 mg, 0.264 mmol, 1.0 eq.), 2-(1-((tert-butyldimethylsilyl)oxy)cyclopropyl)-5-(4,4,5,5-tetramethyl-1,3,2-dioxaborolan-2-yl)pyridine (119 mg, 0.317 mmol, 1.2 eq.), K₂CO₃ (73 mg, 0.528 mmol, 2.0 eq.) in dioxane/H₂O (10 mL, 9:1, v:v) was added Pd(dppf)Cl₂ (19 mg, 0.026 mmol, 0.1 eq.). The mixture was stirred at 100° C. under N₂ atmosphere for 16 hrs. The reaction mixture was concentrated under reduced pressure, and the residue was purified by flash column chromatography on silica gel to give 4-(6-(1-((tert-butyldimethylsilyl)oxy)cyclopropyl)pyridin-3-yl)-5-(cyclopropylmethyl)-2-(2-methyl-2H-indazol-5-yl)-3-oxo-3,5-dihydro-2H-pyrrolo[3,2-c]pyridazine-7-carbonitrile (100 mg, 63%) as a yellow solid. LC-MS (ESI): m/z 592 [M+H]⁺

### Step E: 5-(cyclopropylmethyl)-4-(6-(1-hydroxycyclopropyl)pyridin-3-yl)-2-(2-methyl-2H-indazol-5-yl)-3-oxo-3,5-dihydro-2H-pyrrolo[3,2-c]pyridazine-7-carbonitrile

To a solution of 4-(6-(1-((tert-butyldimethylsilyl)oxy)cyclopropyl)pyridin-3-yl)-5-(cyclopropylmethyl)-2-(2-methyl-2H-indazol-5-yl)-3-oxo-3,5-dihydro-2H-pyrrolo[3,2-c]pyridazine-7-carbonitrile (100 mg, 0.169 mmol) in THE (1 mL) was added a solution of HCl/iPrOH (5 M) (3 mL). The mixture was stirred at rt for 6 hrs. The mixture was quenched with NaHCO₃(sat. aq.) (20 mL) and extracted with DCM (10 mL×3). The organic layer was washed with brine (5 mL), dried over anhydrous Na₂SO₄, filtered and concentrated under reduced pressure. The resulting residue was purified by flash column chromatography on silica gel and RP-prep-HPLC to give 5-(cyclopropylmethyl)-4-(6-(1-hydroxycyclopropyl)pyridin-3-yl)-2-(2-methyl-2H-indazol-5-yl)-3-oxo-3,5-dihydro-2H-pyrrolo[3,2-c]pyridazine-7-carbonitrile (Example 256). ¹H NMR (400 MHz, DMSO-d₆) δ (ppm): 8.72 (s, 1H), 8.52 (d, J=1.6 Hz, 1H), 8.47 (s, 1H), 7.94 (d, J=1.5 Hz, 1H), 7.91 (dd, J=8.2, 2.2 Hz, 1H), 7.82 (d, J=8.1 Hz, 1H), 7.68 (d, J=9.1 Hz, 1H), 7.39 (dd, J=9.2, 2.0 Hz, 1H), 6.28 (s, 1H), 4.21 (s, 3H), 3.39 (d, J=7.1 Hz, 2H), 1.28 (s, 2H), 1.16 (d, J=3.0 Hz, 2H), 0.79-0.68 (m, 1H), 0.39-0.35 (m, 2H), 0.17-0.13 (m, 2H). LC-MS (ESI): m/z 478 [M+H]⁺

### Step F: 5-(cyclopropylmethyl)-4-(6-(3-fluoroprop-1-en-2-yl)pyridin-3-yl)-2-(2-methyl-2H-indazol-5-yl)-3-oxo-3,5-dihydro-2H-pyrrolo[3,2-c]pyridazine-7-carbonitrile

To a solution of 5-(cyclopropylmethyl)-4-(6-(1-hydroxycyclopropyl)pyridin-3-yl)-2-(2-methyl-2H-indazol-5-yl)-3-oxo-3,5-dihydro-2H-pyrrolo[3,2-c]pyridazine-7-carbonitrile (40 mg, 0.084 mmol, 1.0 eq.) in DCM (5 mL) was added DAST (0.033 mL, 0.252 mmol, 3.0 eq.). The mixture was stirred at rt for 4 hrs. The mixture was quenched with NaHCO₃(sat. aq.) (20 mL) and extracted with DCM (10 mL×3). The organic layer was washed with brine (5 mL), dried over anhydrous Na₂SO₄, filtered and concentrated under reduced pressure. The residue was purified by flash column chromatography on silica gel and RP-prep-HPLC to give 5-(cyclopropylmethyl)-4-(6-(3-fluoroprop-1-en-2-yl)pyridin-3-yl)-2-(2-methyl-2H-indazol-5-yl)-3-oxo-3,5-dihydro-2H-pyrrolo[3,2-c]pyridazine-7-carbonitrile (Example 257). ¹H NMR (400 MHz, DMSO-d₆) δ (ppm): 8.74 (s, 1H), 8.69 (d, J=1.6 Hz, 1H), 8.47 (s, 1H), 8.02 (dd, J=8.2, 2.2 Hz, 1H), 7.95 (d, J=1.4 Hz, 1H), 7.88 (d, J=8.2 Hz, 1H), 7.68 (d, J=9.2 Hz, 1H), 7.39 (dd, J=9.2, 2.0 Hz, 1H), 6.28 (s, 1H), 5.73 (s, 1H), 5.55 (s, 1H), 5.43 (s, 1H), 4.21 (s, 3H), 3.40 (d, J=7.1 Hz, 2H), 0.77-0.67 (m, 1H), 0.37-0.33 (m, 2H), 0.15-0.12 (m, 2H). LC-MS (ESI): m/z 480 [M+H]⁺

### Example 258: Synthesis of 5-(cyclopropylmethyl)-2-(2-methyl-2H-indazol-5-yl)-4-(6-methylpyridin-3-yl)-7-(trifluoromethyl)-2,5-dihydro-3H-pyrrolo[3,2-c]pyridazin-3-one

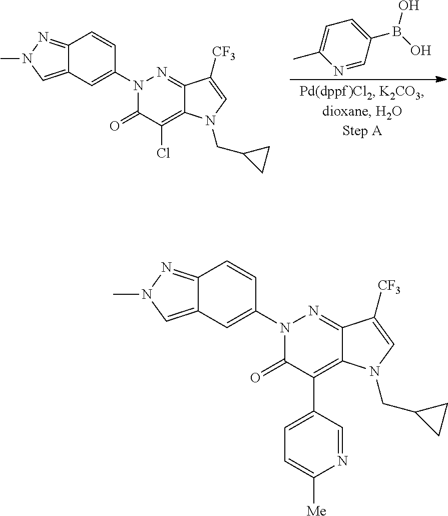

### Step A: 5-(cyclopropylmethyl)-2-(2-methyl-2H-indazol-5-yl)-4-(6-methylpyridin-3-yl)-7-(trifluoromethyl)-2,5-dihydro-3H-pyrrolo[3,2-c]pyridazin-3-one

To a mixture of 4-chloro-5-(cyclopropylmethyl)-2-(2-methyl-2H-indazol-5-yl)-7-(trifluoromethyl)-2,5-dihydro-3H-pyrrolo[3,2-c]pyridazin-3-one (45 mg, 0.1 mmol, 1.0 eq.), (6-methylpyridin-3-yl)boronic acid (18 mg, 0.13 mmol, 1.2 eq.), K₂CO₃ (44 mg, 0.3 mmol, 3.0 eq.) in dioxane (2 mL) and H₂O (0.5 mL) was added Pd(dppf)Cl₂ (8 mg, 0.01 mmol, 0.1 eq.). The mixture was degassed with N₂, sealed under an N₂ atmosphere and heated in a microwave at 100° C. for 1h. The mixture was quenched with H₂O (10 mL) and extracted with EtOAc (10 mL×3). The separated organic layer was washed with brine (20 mL), dried over Na₂SO₄ and evaporated to dryness. The resulting residue was purified by flash column chromatography on silica gel and Prep-TLC to give 5-(cyclopropylmethyl)-2-(2-methyl-2H-indazol-5-yl)-4-(6-methylpyridin-3-yl)-7-(trifluoromethyl)-2,5-dihydro-3H-pyrrolo[3,2-c]pyridazin-3-one. ¹H NMR (400 MHz, DMSO-d₆) δ (ppm): 8.56 (d, J=1.8 Hz, 1H), 8.46 (s, 1H), 8.43 (s, 1H), 7.90 (d, J=1.3 Hz, 1H), 7.86 (dd, J=7.9, 2.2 Hz, 1H), 7.67 (d, J=9.2 Hz, 1H), 7.43-7.33 (m, 2H), 4.21 (s, 3H), 3.42 (s, 2H), 2.55 (s, 3H), 0.70-0.58 (m, 1H), 0.38-0.29 (m, 2H), 0.18-0.10 (m, 2H). LC-MS (ESI): m/z 479 [M+H]⁺.

### Example 259: Synthesis of 2-(5-(cyclopropylmethyl)-4-(6-cyclopropylpyridin-3-yl)-2-(2-methyl-2H-indazol-5-yl)-3-oxo-3,5-dihydro-2H-pyrrolo[3,2-c]pyridazin-7-yl)acetonitrile

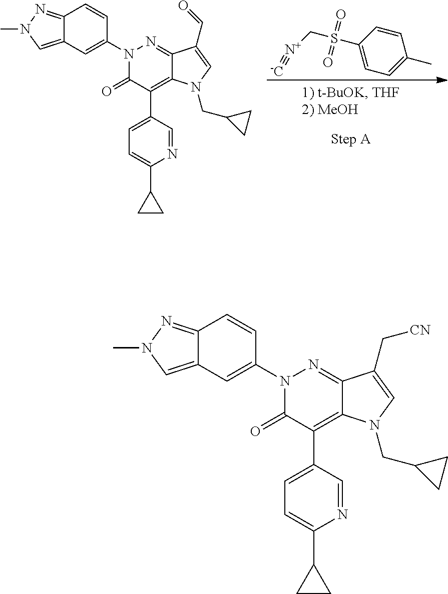

### Step A: 2-(5-(cyclopropylmethyl)-4-(6-cyclopropylpyridin-3-yl)-2-(2-methyl-2H-indazol-5-yl)-3-oxo-3,5-dihydro-2H-pyrrolo[3,2-c]pyridazin-7-yl)acetonitrile

To a solution of 5-(cyclopropylmethyl)-4-(6-cyclopropylpyridin-3-yl)-2-(2-methyl-2H-indazol-5-yl)-3-oxo-2H,3H,5H-pyrrolo[3,2-c]pyridazine-7-carbaldehyde (50 mg, 0.108 mmol, 1.0 eq.) in THE (2 mL) was added (tert-butoxy)potassium (30.2 mg, 0.269 mmol, 2.5 eq.) at −65° C. The reaction was stirred at this temperature for an additional 10 min and then slowly allowed to warm to room temperature (˜1 hr). The resulting mixture was refluxed at 85° C. for 16 hrs. After cooling to room temperature, the reaction was quenched with adding H₂O (10 mL) and extracted with EtOAc (10 mL×3). The organic layers were combined and washed with brine (20 mL), dried over MgSO₄, filtered and remove the solvents under reduced pressure, the residue was purified by flash column chromatography and prep-TLC to give 2-[5-(cyclopropylmethyl)-4-(6-cyclopropylpyridin-3-yl)-2-(2-methyl-2H-indazol-5-yl)-3-oxo-2H,3H,5H-pyrrolo[3,2-c]pyridazin-7-yl]acetonitrile.

¹H NMR (400 MHz, DMSO-d₆) δ (ppm): 8.47 (d, J=1.7 Hz, 1H), 8.46 (s, 1H), 7.90 (d, J=1.3 Hz, 1H), 7.78 (dd, J=8.0, 2.2 Hz, 1H), 7.73 (s, 1H), 7.66 (d, J=9.1 Hz, 1H), 7.42 (d, J=8.0 Hz, 1H), 7.38 (dd, J=9.1, 2.0 Hz, 1H), 4.20 (s, 3H), 3.95 (s, 2H), 3.32 (d, J=7.2 Hz, 2H), 2.21-2.10 (m, 1H), 1.03-0.96 (m, 4H), 0.70-0.58 (m, 1H), 0.35-0.27 (m, 2H), 0.10-0.06 (m, 2H). LC-MS (ESI): m/z 476 [M+H]⁺.

### Example 260: Synthesis of 5-(cyclopropylmethyl)-4-(6-cyclopropylpyridin-3-yl)-7,7-dimethyl-2-(2-methyl-2H-indazol-5-yl)-2,5,6,7-tetrahydro-3H-pyrrolo[3,2-c]pyridazin-3-one (Example 260)

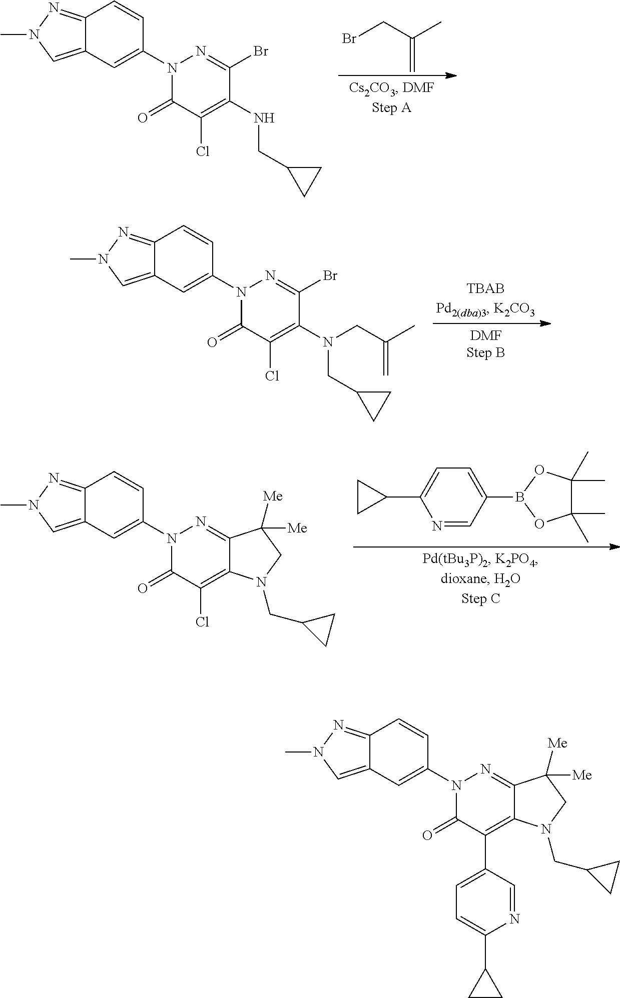

### Step A: 6-bromo-4-chloro-5-((cyclopropylmethyl)(2-methylallyl)amino)-2-(2-methyl-2H-indazol-5-yl)pyridazin-3(2H)-one

To a solution of 6-bromo-4-chloro-5-((cyclopropylmethyl)amino)-2-(2-methyl-2H-indazol-5-yl)pyridazin-3(2H)-one (500 mg, 1.2 mmol, 1.0 eq.) in DMF (7 mL) was added Cs₂CO₃ (996 mg, 3.06 mmol, 2.5 eq.) and 3-bromo-2-methylprop-1-ene (247 mg, 1.84 mmol, 1.5 eq.) at room temperature. The reaction mixture was stirred at 50° C. for 2 h. The resulting mixture was poured into ice water (10 mL) and extracted with EtOAc (10 mL×3). The organic layers were combined and washed with brine (20 mL), dried over anhydrous Na₂SO₄, filtered, the solvent was removed under reduced pressure, and the residue was purified by the flash column of chromatography on silica gel to give 6-bromo-4-chloro-5-[(cyclopropylmethyl)(2-methylprop-2-en-1-yl)amino]-2-(2-methyl-2H-indazol-5-yl)-2,3-dihydropyridazin-3-one (400 mg, 70%) as a yellow oil. LC-MS (ESI): m/z 462 [M+H]⁺.

### Step B: 4-chloro-5-(cyclopropylmethyl)-7,7-dimethyl-2-(2-methyl-2H-indazol-5-yl)-2,5,6,7-tetrahydro-3H-pyrrolo[3,2-c]pyridazin-3-one

To a solution of 6-bromo-4-chloro-5-[(cyclopropylmethyl)(2-methylprop-2-en-1-yl)amino]-2-(2-methyl-2H-indazol-5-yl)-2,3-dihydropyridazin-3-one (400 mg, 0.16 mmol, 1.0 eq.), TBAB (278 mg, 0.86 mmol, 1.0 eq.), K₂CO₃ (298 mg, 2.16 mmol, 2.5 eq.) in DMF (5 mL) was added Pd₂(dba)₃ (78 mg, 0.09 mmol, 0.1 eq.). The reaction mixture was stirred at 100° C. for 15 hrs. The resulting mixture was concentrated under reduced pressure, and the residue was purified by flash column chromatography on silica gel to give 4-chloro-5-(cyclopropylmethyl)-7,7-dimethyl-2-(2-methyl-2H-indazol-5-yl)-2,5,6,7-tetrahydro-3H-pyrrolo[3,2-c]pyridazin-3-one (100 mg, 30%) as a light yellow solid. LC-MS (ESI): m/z 384 [M+H]⁺

### Step C: 5-(cyclopropylmethyl)-4-(6-cyclopropylpyridin-3-yl)-7,7-dimethyl-2-(2-methyl-2H-indazol-5-yl)-2,5,6,7-tetrahydro-3H-pyrrolo[3,2-c]pyridazin-3-one

To a solution of 4-chloro-5-(cyclopropylmethyl)-7,7-dimethyl-2-(2-methyl-2H-indazol-5-yl)-2,5,6,7-tetrahydro-3H-pyrrolo[3,2-c]pyridazin-3-one (50 mg, 0.13 mmol, 1.0 eq.) and 2-cyclopropyl-5-(tetramethyl-1,3,2-dioxaborolan-2-yl)pyridine (47 mg, 0.20 mmol, 1.5 eq.) in dioxane/H₂O (5 mL, 4:1, v:v) was added Pd(tBu₃P)₂ (13 mg, 0.03 mmol, 0.3 eq.) and K₃PO₄ (82 mg, 0.39 mmol, 3.0 eq.). The reaction mixture was stirred at 100° C. for 15 hrs. The resulting mixture was concentrated under reduced pressure, and the residue was purified by flash column chromatography on silica gel and prep-HPLC to give 5-(cyclopropylmethyl)-4-(6-cyclopropylpyridin-3-yl)-7,7-dimethyl-2-(2-methyl-2H-indazol-5-yl)-2,5,6,7-tetrahydro-3H-pyrrolo[3,2-c]pyridazin-3-one. ¹H NMR (400 MHz, DMSO-d₆) δ (ppm): 8.37 (s, 1H), 8.32 (s, 1H), 7.75 (s, 1H), 7.59-7.56 (m, 2H), 7.32-7.28 (m, 2H), 4.18 (s, 3H), 3.56 (s, 2H), 2.69 (d, J=6.8 Hz, 2H), 2.13-2.07 (m, 1H), 1.36 (s, 6H), 0.99-0.93 (m, 4H), 0.76-0.68 (m, 1H), 0.36-0.35 (m, 2H), 0.15-0.09 (m, 2H). LC-MS (ESI): m/z 467 [M+H]⁺.

### Example 261: Synthesis of 5-(cyclopropylmethyl)-4-(6-cyclopropylpyridin-3-yl)-2-(2-methyl-2H-indazol-5-yl)-7-(methylsulfonyl)-2,5-dihydro-3H-pyrrolo[3,2-c]pyridazin-3-one

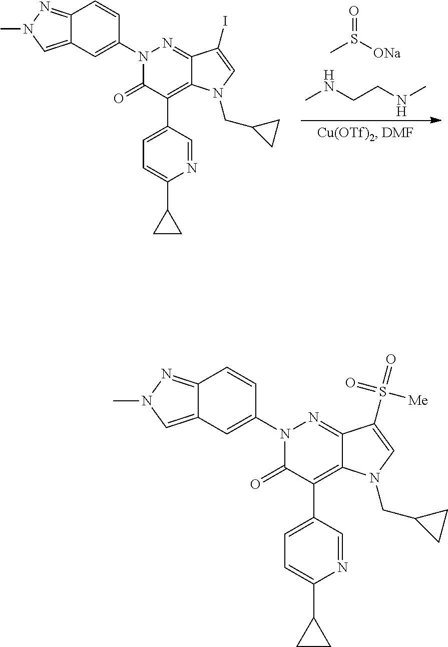

To a solution of 5-(cyclopropylmethyl)-4-(6-cyclopropylpyridin-3-yl)-7-iodo-2-(2-methyl-2H-indazol-5-yl)-2H,3H,5H-pyrrolo[3,2-c]pyridazin-3-one (100 mg, 0.178 mmol, 1.0 eq.) in DMF (2 mL) was added sodium methanesulfinate (36 mg, 0.356 mmol, 2.0 eq.), methyl[2-(methylamino)ethyl]amine (8 μL, 0.071 mmol, 0.4 eq.) and Cu(OTf)₂ (13 mg, 0.036 mmol, 0.2 eq.) at room temperature. The resulting mixture was heated to 105° C. under microwave irradiation for 30 min. After cooling, the reaction was quenched by adding H₂O (5 mL) and extracted with EtOAc (10 mL×3). The organic layers were combined and washed with brine (20 mL), dried over MgSO₄, filtered and concentrated under reduced pressure. The residue was purified by flash column chromatography on silica gel and RP-prep-HPLC to give 5-(cyclopropylmethyl)-4-(6-cyclopropylpyridin-3-yl)-7-methanesulfonyl-2-(2-methyl-2H-indazol-5-yl)-2H,3H,5H-pyrrolo[3,2-c]pyridazin-3-one. ¹H NMR (400 MHz, DMSO-d₆) δ (ppm): 8.51 (d, J=1.7 Hz, 1H), 8.47 (s, 1H), 8.44 (s, 1H), 7.96 (d, J=1.3 Hz, 1H), 7.81 (dd, J=8.0, 2.2 Hz, 1H), 7.68 (d, J=9.2 Hz, 1H), 7.48-7.36 (m, 2H), 4.21 (s, 3H), 3.43 (d, J=7.1 Hz, 2H), 3.29 (s, 3H), 2.21-2.16 (m, 1H), 1.06-0.94 (m, 4H), 0.73-0.61 (m, 1H), 0.35-0.31 (m, 2H), 0.14-0.11 (m, 2H). LC-MS (ESI): m/z 515 [M+H]⁺.

### Example 262: Synthesis of 5-(cyclopropylmethyl)-4-(4-cyclopropylphenyl)-2-(2-methyl-2H-indazol-5-yl)-7-(methylsulfonyl)-2,5-dihydro-3H-pyrrolo[3,2-c]pyridazin-3-one

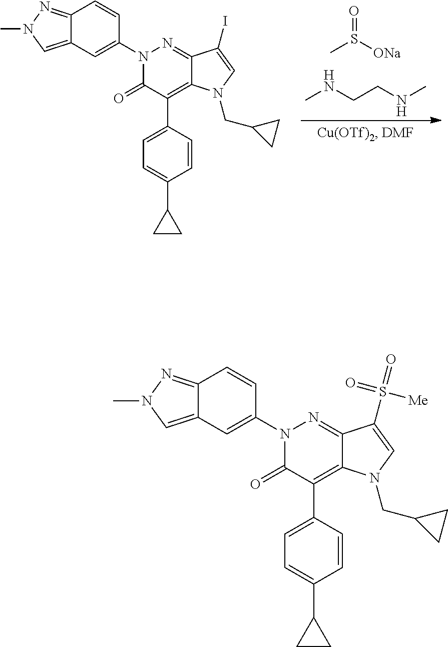

To a solution of 5-(cyclopropylmethyl)-4-(4-cyclopropylphenyl)-7-iodo-2-(2-methyl-2H-indazol-5-yl)-2,5-dihydro-3H-pyrrolo[3,2-c]pyridazin-3-one (100 mg, 0.178 mmol, 1.0 eq.) in DMF (2 mL) was added sodium methanesulfinate (36 mg, 0.356 mmol, 2.0 eq.), N¹,N²-dimethylethane-1,2-diamine (7 mg, 0.071 mmol, 0.4 eq.) and Cu(OTf)₂ (13 mg, 0.036 mmol, 0.2 eq.). The reaction was stirred under microwave irradiation at 105° C. for 30 min. After cooling, the mixture was poured into H₂O (20 mL) and extracted with DCM (10 mL×3). The organic layers were combined and washed with ammonia solution (1N) (10 ml×2) then brine (20 mL), dried over anhydrous Na₂SO₄, filtered and evaporated under reduced pressure. The residue was purified by flash column chromatography on silica gel and RP-prep-HPLC to afford 5-(cyclopropylmethyl)-4-(4-cyclopropylphenyl)-2-(2-methyl-2H-indazol-5-yl)-7-(methylsulfonyl)-2,5-dihydro-3H-pyrrolo[3,2-c]pyridazin-3-one. ¹H NMR (400 MHz, DMSO-d₆) δ (ppm): 8.47 (s, 1H), 8.41 (s, 1H), 7.95 (d, J=1.3 Hz, 1H), 7.67 (d, J=9.2 Hz, 1H), 7.40 (dd, J=9.2, 1.9 Hz, 1H), 7.36 (d, J=8.1 Hz, 2H), 7.19 (d, J=8.2 Hz, 2H), 4.21 (s, 3H), 3.42 (d, J=7.2 Hz, 2H), 3.28 (s, 3H), 2.11-1.93 (m, 1H), 1.12-0.94 (m, 2H), 0.77-0.64 (m, 3H), 0.32-0.27 (m, 2H), 0.10-0.05 (m, 2H). LC-MS (ESI): m/z 514 [M+H]⁺.

### Example 263: Preparation of 5-(cyclopropylmethyl)-4-(6-cyclopropylpyridin-3-yl)-6-methyl-2-(2-methyl-2H-indazol-5-yl)-2,5-dihydro-3H-pyrrolo[3,2-c]pyridazin-3-one

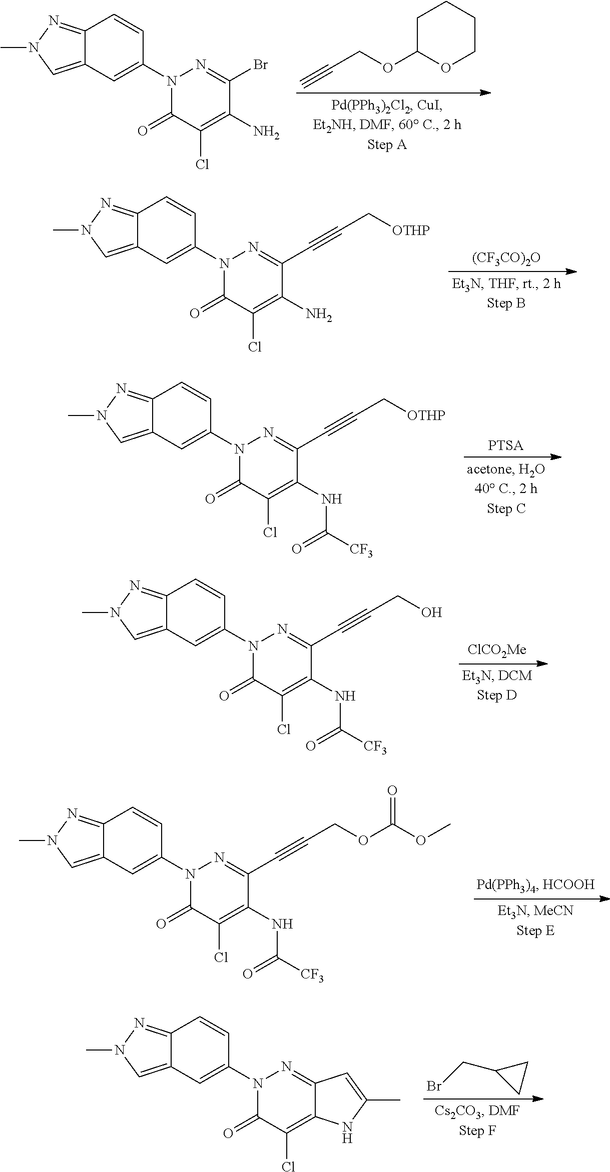

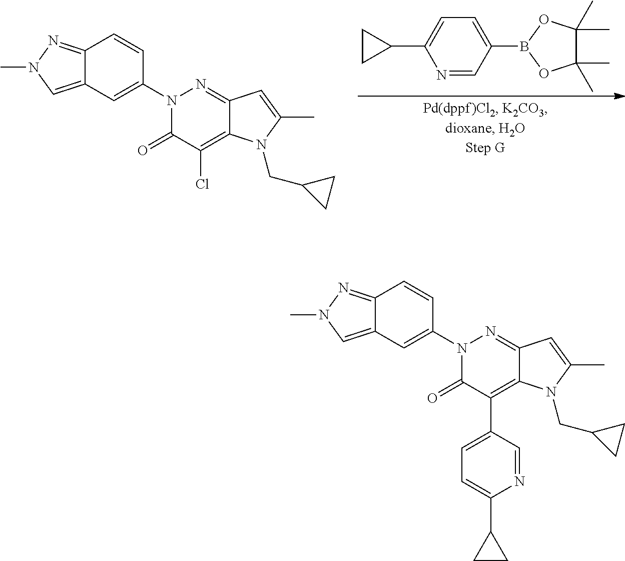 [SMILES: Cc1cc2nn(-c3ccc4nn(C)cc4c3)c(=O)c(Cl)c2n1CC1CC1 ; CC1(C)OB(c2ccc(C3CC3)nc2)OC1(C)C ; Cc1cc2nn(-c3ccc4nn(C)cc4c3)c(=O)c(-c3ccc(C4CC4)nc3)c2n1CC1CC1]

### Step A: 5-amino-4-chloro-2-(2-methyl-2H-indazol-5-yl)-6-(3-((tetrahydro-2H-pyran-2-yl)oxy)prop-1-yn-1-yl)pyridazin-3(2H)-one

5-amino-6-bromo-4-chloro-2-(2-methyl-2H-indazol-5-yl)-2,3-dihydropyridazin-3-one(300 mg, 0.846 mmol, 1.0 eq.), PdCl₂(PPh₃)₂ (9.0 mg, 0.013 mmol, 0.015 eq.), Et₂NH (433 mg, 5.922 mmol, 7.0 eq.) and CuI (2 mg, 0.013 mmol, 0.015 eq.) were added to DMF (6 mL) and the mixture was stirred under argon for 15 min. Then 2-(prop-2-yn-1-yloxy)oxane (130 mg, 0.931 mmol, 1.2 eq.) was added and the resulting reaction mixture was stirred at 60° C. for 3 h. After cooling, the reaction mixture was diluted with EtOAc (10 mL), washed twice with NH₄Cl solution, dried over Na₂SO₄ and concentrated under reduced pressure. The residue was purified by flash column to give 5-amino-4-chloro-2-(2-methyl-2H-indazol-5-yl)-6-(3-((tetrahydro-2H-pyran-2-yl)oxy)prop-1-yn-1-yl)pyridazin-3(2H)-one (200 mg, 57%) as yellow solid. LC-MS (ESI): m/z 414 [M+H]⁺.

### Step B: N-(5-chloro-1-(2-methyl-2H-indazol-5-yl)-6-oxo-3-(3-((tetrahydro-2H-pyran-2-yl)oxy)prop-1-yn-1-yl)-1,6-dihydropyridazin-4-yl)-2,2,2-trifluoroacetamide

To a mixture of 5-amino-4-chloro-2-(2-methyl-2H-indazol-5-yl)-6-(3-((tetrahydro-2H-pyran-2-yl)oxy)prop-1-yn-1-yl)pyridazin-3(2H)-one (200 mg, 0.484 mmol, 1.0 eq.) and TEA (0.202 mL, 1.452 mmol, 3.0 eq.) in THF (10 mL) was added dropwise trifluoroacetic anhydride (0.101 mL, 0.726 mmol, 1.5.0 eq.), and the solution was stirred at rt for 3 hours. After cooling, the solution was diluted with EtOAc, washed twice with a saturated NaHCO₃solution, dried over Na₂SO₄ and concentrated under reduced pressure. The residue was purified by flash column to give N-(5-chloro-1-(2-methyl-2H-indazol-5-yl)-6-oxo-3-(3-((tetrahydro-2H-pyran-2-yl)oxy)prop-1-yn-1-yl)-1,6-dihydropyridazin-4-yl)-2,2,2-trifluoroacetamide (200 mg, 81%) as yellow solid. LC-MS (ESI): m/z 510 [M+H]⁺.

### Step C: N-(5-chloro-3-(3-hydroxyprop-1-yn-1-yl)-1-(2-methyl-2H-indazol-5-yl)-6-oxo-1,6-dihydropyridazin-4-yl)-2,2,2-trifluoroacetamide

To a solution of N-(5-chloro-1-(2-methyl-2H-indazol-5-yl)-6-oxo-3-(3-((tetrahydro-2H-pyran-2-yl)oxy)prop-1-yn-1-yl)-1, 6-dihydropyridazin-4-yl)-2,2,2-trifluoroacetamide (200 mg, 0.393 mmol, 1.0 eq.) in H₂O (4 mL) and acetone (4 mL) was added pTSA (67 mg, 0.393 mmol, 1.0 eq.). The mixture was stirred at 40° C. for 2 hrs. The mixture was concentrated, and the residue was purified by flash column to give N-(5-chloro-3-(3-hydroxyprop-1-yn-1-yl)-1-(2-methyl-2H-indazol-5-yl)-6-oxo-1,6-dihydropyridazin-4-yl)-2,2,2-trifluoroacetamide (150 mg, 90%) as a yellow solid. LC-MS (ESI): m/z 426 [M+H]⁺.

### Step D: 3-(5-chloro-1-(2-methyl-2H-indazol-5-yl)-6-oxo-4-(2,2,2-trifluoroacetamido)-1,6-dihydropyridazin-3-yl)prop-2-yn-1-yl Methyl Carbonate

To a solution of N-(5-chloro-3-(3-hydroxyprop-1-yn-1-yl)-1-(2-methyl-2H-indazol-5-yl)-6-oxo-1,6-dihydropyridazin-4-yl)-2,2,2-trifluoroacetamide (150 mg, 0.353 mmol, 1.0 eq.) and TEA (178.55 mg, 1.764 mmol, 5.0 eq.) in DCM (5 mL) was added dropwise methyl chloroformate (66.70 mg, 0.706 mmol, 2.0 eq.), and the solution was stirred at rt for 3 hrs. The solution was diluted with DCM (20 mL), washed twice with a saturated NaHCO₃solution, dried over Na₂SO₄ and concentrated under reduced pressure. The residue was purified by flash column to give 3-(5-chloro-1-(2-methyl-2H-indazol-5-yl)-6-oxo-4-(2,2,2-trifluoroacetamido)-1,6-dihydropyridazin-3-yl)prop-2-yn-1-yl methyl carbonate (120 mg, 70%) as yellow solid. LC-MS (ESI): m/z 484 [M+H]⁺.

### Step E: 4-chloro-6-methyl-2-(2-methyl-2H-indazol-5-yl)-2H,3H,5H-pyrrolopyridazin-3-one

To a mixture of 3-(5-chloro-1-(2-methyl-2H-indazol-5-yl)-6-oxo-4-(2,2,2-trifluoroacetamido)-1,6-dihydropyridazin-3-yl)prop-2-yn-1-yl methyl carbonate (120 mg, 0.248 mmol, 1.0 eq.), Pd(PPh₃)₄ (14.0 mg, 0.012 mmol, 0.05 eq.) and TEA (0.1 mL, 0.744 mmol, 3.0 eq.) in MeCN (5 mL) was added formic acid (95.0 mg, 0.496 mmol, 2.0 eq.) and the mixture was stirred at 80° C. under argon for 5 hrs. The reaction mixture was concentrated under reduced pressure. The residue was purified by flash column to give 4-chloro-6-methyl-2-(2-methyl-2H-indazol-5-yl)-2H,3H,5H-pyrrolopyridazin-3-one (40 mg, 51%) as yellow solid. LC-MS (ESI): m/z 314 [M+H]⁺.

### Step F: 4-chloro-5-(cyclopropylmethyl)-6-methyl-2-(2-methyl-2H-indazol-5-yl)-2H,3H,5H-pyrrolopyridazin-3-one

To a mixture of 4-chloro-6-methyl-2-(2-methyl-2H-indazol-5-yl)-2H,3H,5H-pyrrolopyridazin-3-one (40 mg, 0.127 mmol, 1.0 eq.) and (bromomethyl)cyclopropane (0.02 mL, 0.191 mmol, 1.5 eq.) in DMF (5 mL) was added Cs₂CO₃ (83.0 mg, 0.255 mmol, 2.0 eq.) at room temperature. The mixture was stirred for 5 hrs before being quenched with H₂O (10 mL), extracted with EA (10 mL×3). The organic layer was washed with brine (20 mL), dried over anhydrous Na₂SO₄, concentrated and the residue was purified by flash column to give 4-chloro-5-(cyclopropylmethyl)-6-methyl-2-(2-methyl-2H-indazol-5-yl)-2H,3H,5H-pyrrolopyridazin-3-one (30 mg, 64%) as yellow solid. LC-MS (ESI): m/z 368 [M+H]⁺.

### Step G: 5-(cyclopropylmethyl)-4-(6-cyclopropylpyridin-3-yl)-6-methyl-2-(2-methyl-2H-indazol-5-yl)-2H,3H,5H-pyrrolopyridazin-3-one

To a solution of 4-chloro-5-(cyclopropylmethyl)-6-methyl-2-(2-methyl-2H-indazol-5-yl)-2H,3H,5H-pyrrolopyridazin-3-one (30 mg, 0.082 mmol, 1.0 eq.), 2-cyclopropyl-5-(tetramethyl-1,3,2-dioxaborolan-2-yl)pyridine (24.0 mg, 0.098 mmol, 1.2 eq.) and K₂CO₃ (23.0 mg, 0.163 mmol, 2.0 eq.) in dioxane/H₂O (5 mL, 4:1, v:v) was added Pd(dppf)Cl₂ (3.0 mg, 0.004 mmol, 0.05 eq.). The mixture was stirred at 100° C. under N₂ atmosphere for 14 hrs. The mixture was concentrated and purified by RP-prep-HPLC to give 5-(cyclopropylmethyl)-4-(6-cyclopropylpyridin-3-yl)-6-methyl-2-(2-methyl-2H-indazol-5-yl)-2H,3H,5H-pyrrolopyridazin-3-one. ¹H NMR (400 MHz, DMSO-d₆) δ (ppm): 8.43 (d, J=1.6 Hz, 1H), 8.40 (s, 1H), 7.86 (s, 1H), 7.73 (dd, J=8.0, 2.0 Hz, 1H), 7.61 (d, J=9.1 Hz, 1H), 7.39 (d, J=8.0 Hz, 1H), 7.35 (dd, J=9.1, 1.7 Hz, 1H), 6.25 (s, 1H), 4.18 (s, 3H), 3.40 (d, J=5.5 Hz, 2H), 2.33 (s, 3H), 2.21-2.13 (m, 1H), 1.05-0.90 (m, 4H), 0.65-0.53 (m, 1H), 0.24-0.18 (m, 2H), −0.1-−0.12 (m, 2H). LC-MS (ESI): m/z 451 [M+H]⁺.

### Example 264: Synthesis of 5-(cyclopropylmethyl)-4-(4-(difluoromethoxy)phenyl)-6-methyl-2-(2-methyl-2H-indazol-5-yl)-3-oxo-3,5-dihydro-2H-pyrrolo[3,2-c]pyridazine-7-carbonitrile

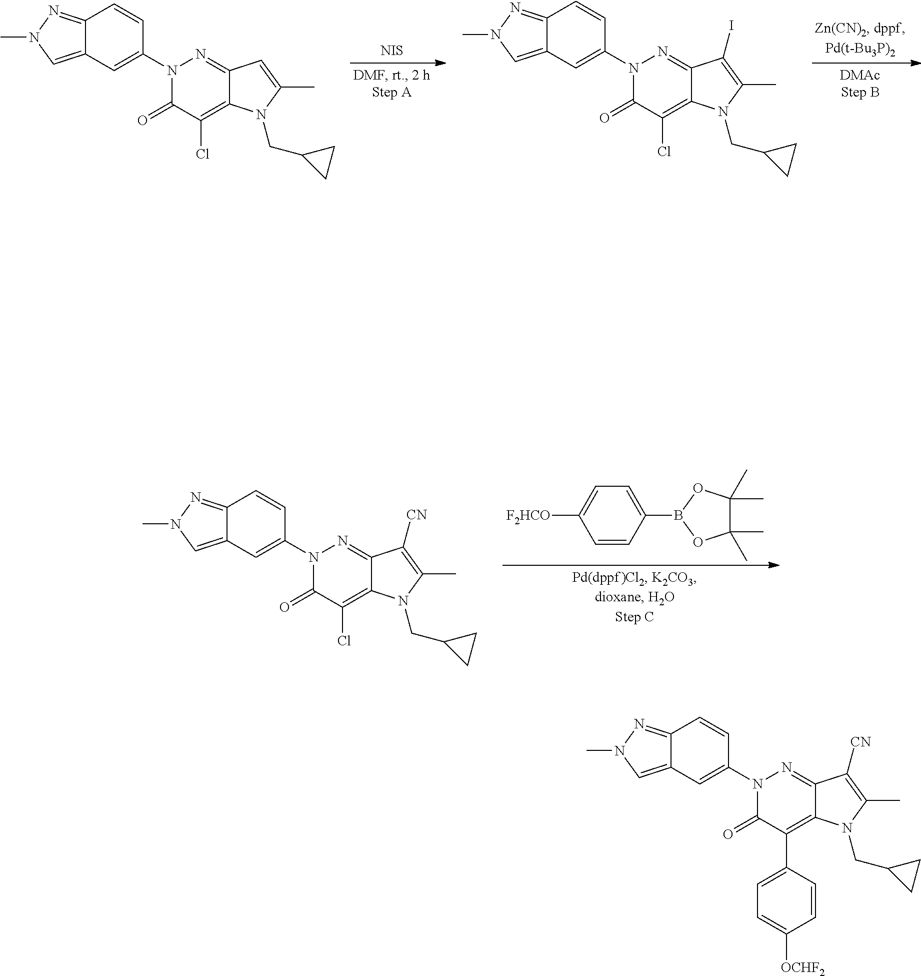 [SMILES: Cc1cc2nn(-c3ccc4nn(C)cc4c3)c(=O)c(Cl)c2n1CC1CC1 ; "NIS" ; Cc1c(I)c2nn(-c3ccc4nn(C)cc4c3)c(=O)c(Cl)c2n1CC1CC1 ; Cc1c(C#N)c2nn(-c3ccc4nn(C)cc4c3)c(=O)c(Cl)c2n1CC1CC1 ; CC1(C)OB(c2ccc(OC(F)F)cc2)OC1(C)C ; Cc1c(C#N)c2nn(-c3ccc4nn(C)cc4c3)c(=O)c(-c3ccc(OC(F)F)cc3)c2n1CC1CC1]

### Step A: 4-chloro-5-(cyclopropylmethyl)-7-iodo-6-methyl-2-(2-methyl-2H-indazol-5-yl)-2,5-dihydro-3H-pyrrolo[3,2-c]pyridazin-3-one

To a solution of 4-chloro-5-(cyclopropylmethyl)-6-methyl-2-(2-methyl-2H-indazol-5-yl)-2H,3H,5H-pyrrolopyridazin-3-one (200 mg, 0.544 mmol, 1.0 eq.) in anhydrous DMF (15 mL) was added NIS (147 mg, 0.653 mmol, 1.2 eq.) at rt. The reaction mixture was stirred at rt for 2 hrs under N₂ atmosphere. The mixture was then poured into ice water (20 mL) and extracted with EtOAc (15 mL×3 mL). The organic layers were combined and washed with brine (20 ml), filtered and concentrated under reduced pressure. The resulting residue was purified by flash column chromatography to give 4-chloro-5-(cyclopropylmethyl)-7-iodo-6-methyl-2-(2-methyl-2H-indazol-5-yl)-2H,3H,5H-pyrrolopyridazin-3-one (220 mg, 82%) as a yellow solid. LC-MS (ESI): m/z 494 [M+H]⁺.

### Step B: 4-chloro-5-(cyclopropylmethyl)-6-methyl-2-(2-methyl-2H-indazol-5-yl)-3-oxo-3,5-dihydro-2H-pyrrolo[3,2-c]pyridazine-7-carbonitrile

To a mixture of 4-chloro-5-(cyclopropylmethyl)-7-iodo-6-methyl-2-(2-methyl-2H-indazol-5-yl)-2H,3H,5H-pyrrolopyridazin-3-one (220 mg, 0.446 mmol, 1.0 eq.), Zn(CN)₂ (105 mg, 0.891 mmol, 2.0 eq.) and DPPF (25 mg, 0.045 mmol, 0.1 eq.) in DMAc (8 mL) was added Pd(tBu₃)₂ (23 mg, 0.045 mmol, 0.1 eq.) at room temperature. The resulting mixture was kept stirring at 100° C. under N₂ atmosphere for 14 hrs. After cooling, the reaction was quenched by adding H₂O (5 mL), extracted with EtOAc (10 mL×3), and the organic layers were combined and washed with brine (20 mL), filtered and concentrated under reduced pressure. The resulting residue was purified by flash column chromatography to give 4-chloro-5-(cyclopropylmethyl)-6-methyl-2-(2-methyl-2H-indazol-5-yl)-3-oxo-2H,3H,5H-pyrrolopyridazine-7-carbonitrile (150 mg, 86%) as a yellow solid. LC-MS (ESI): m/z 393 [M+H]⁺.

### Step C: 5-(cyclopropylmethyl)-4-(4-(difluoromethoxy)phenyl)-6-methyl-2-(2-methyl-2H-indazol-5-yl)-3-oxo-3,5-dihydro-2H-pyrrolo[3,2-c]pyridazine-7-carbonitrile

To a mixture of 4-chloro-5-(cyclopropylmethyl)-6-methyl-2-(2-methyl-2H-indazol-5-yl)-3-oxo-2H,3H,5H-pyrrolopyridazine-7-carbonitrile (40 mg, 0.102 mmol, 1.0 eq.), 2-(4-(difluoromethoxy)phenyl)-4,4,5,5-tetramethyl-1,3,2-dioxaborolane (41 mg, 0.153 mmol, 1.5 eq.) and K₂CO₃ (28 mg, 0.204 mmol, 2.0 eq.) in dioxane/H₂O (4 mL, 3:1, v:v) was added Pd(tBu₃)₂ (5 mg, 0.010 mmol, 0.1 eq.) at room temperature. The resulting mixture was kept stirring at 100° C. under N₂ atmosphere for 14 hrs. After cooling, the reaction was quenched by adding H₂O (5 mL), extracted with EtOAc (10 mL×3), the organic layers were combined and washed with brine (20 mL), filtered and concentrated under reduced pressure, and the residue was purified by flash column chromatography and RP-prep-HPLC to give 5-(cyclopropylmethyl)-4-(6-cyclopropylpyridin-3-yl)-6-methyl-2-(2-methyl-2H-indazol-5-yl)-3-oxo-2H,3H,5H-pyrrolopyridazine-7-carbonitrile. ¹H NMR (400 MHz, DMSO-d₆) δ (ppm): 7.92 (d, J=1.3 Hz, 1H), 7.65 (d, J=9.2 Hz, 1H), 7.54 (d, J=8.7 Hz, 2H), 7.53 (s, 1H), 7.37 (dd, J=9.2, 2.0 Hz, 1H), 7.34 (t, J_{HF}=72.2 Hz, 1H), 7.30 (d, J=8.6 Hz, 2H), 4.20 (s, 3H), 3.49 (d, J=6.7 Hz, 2H), 2.55 (s, 3H), 0.73-0.60 (m, 1H), 0.30-0.20 (m, 2H), −0.02-−0.09 (m, 2H). LC-MS (ESI): m/z 501 [M+H]⁺.

### Example 265: Synthesis of 5-(cyclopropylmethyl)-4-(6-cyclopropylpyridin-3-yl)-6-methyl-2-(2-methyl-2H-indazol-5-yl)-3-oxo-2H,3H,5H-pyrrolopyridazine-7-carbonitrile

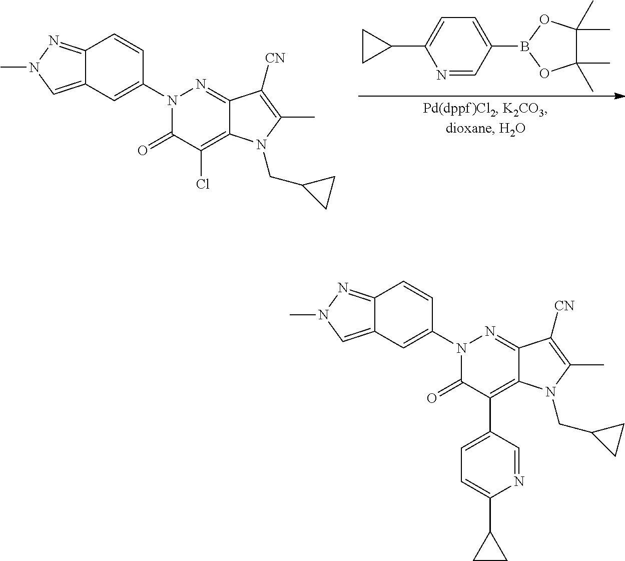 [SMILES: Cc1c(C#N)c2nn(-c3ccc4nn(C)cc4c3)c(=O)c(Cl)c2n1CC1CC1 ; CC1(C)OB(c2ccc(C3CC3)nc2)OC1(C)C ; Cc1c(C#N)c2nn(-c3ccc4nn(C)cc4c3)c(=O)c(-c3ccc(C4CC4)nc3)c2n1CC1CC1]

To a solution of 4-chloro-5-(cyclopropylmethyl)-6-methyl-2-(2-methyl-2H-indazol-5-yl)-3-oxo-2H,3H,5H-pyrrolopyridazine-7-carbonitrile (40 mg, 0.102 mmol, 1.0 eq.), K₂CO₃ (28.2 mg, 0.204 mmol, 2.0 eq.) and 2-cyclopropyl-5-(tetramethyl-1,3,2-dioxaborolan-2-yl)pyridine (37.4 mg, 0.153 mmol, 1.5 eq.) in dioxane/H₂O (3 mL, 5/1, v:v) was added Pd(tBu₃)₂ (5.0 mg, 0.010 mmol, 0.1 eq.) at room temperature. The mixture was stirred at 100° C. under N₂ atmosphere overnight. After cooling, the resulting mixture was poured into ice water (10 mL), extracted with EtOAc (10 mL×3), the organic layers were combined and washed with brine (20 mL), dried over anhydrous Na₂SO₄, filtered and concentrated under reduced pressure, and the residue was purified by flash column chromatography on silica gel and prep-TLC to give 5-(cyclopropylmethyl)-4-(6-cyclopropylpyridin-3-yl)-6-methyl-2-(2-methyl-2H-indazol-5-yl)-3-oxo-2H,3H,5H-pyrrolopyridazine-7-carbonitrile. ¹H NMR (400 MHz, DMSO-d₆) δ (ppm): 8.46 (d, J=1.6 Hz, 1H), 8.44 (s, 1H), 7.92 (d, J=1.3 Hz, 1H), 7.77 (dd, J=8.0, 2.1 Hz, 1H), 7.66 (d, J=9.1 Hz, 1H), 7.42 (d, J=8.1 Hz, 1H), 7.37 (dd, J=9.1, 1.8 Hz, 1H), 4.20 (s, 3H), 3.51 (s, 2H), 2.55 (s, 3H), 2.23-2.13 (m, 1H), 1.09-0.91 (m, 4H), 0.69-0.58 (m, 1H), 0.27-0.23 (m, 2H), −0.1-0.0 (m, 2H). LC-MS (ESI): m/z 476 [M+H]+.

### Example 266: Preparation of 5-(cyclopropylmethyl)-6-methyl-2-(2-methyl-2H-indazol-5-yl)-4-(6-methylpyridin-3-yl)-3-oxo-2H,3H,5H-pyrrolopyridazine-7-carbonitrile

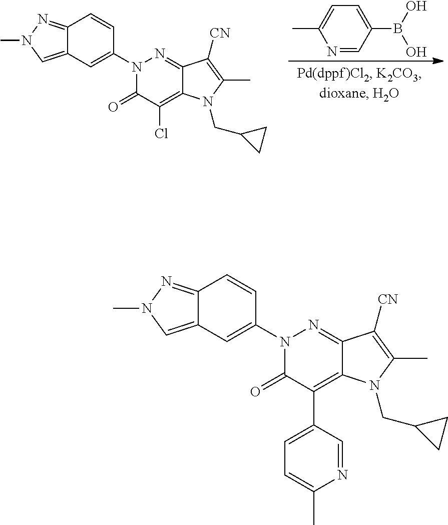

To a mixture of 4-chloro-5-(cyclopropylmethyl)-6-methyl-2-(2-methyl-2H-indazol-5-yl)-3-oxo-2H,3H,5H-pyrrolopyridazine-7-carbonitrile (40 mg, 0.102 mmol, 1.0 eq.), 2-methyl-5-(tetramethyl-1,3,2-dioxaborolan-2-yl)pyridine (44.7 mg, 0.204 mmol, 1.5 eq.) and K₂CO₃ (28.2 mg, 0.204 mmol, 2.0 eq.) in dioxane/H₂O (3 mL, 5:1, v:v) was added Pd(tBu₃)₂ (5.2 mg, 0.010 mmol, 0.1 eq.) at room temperature. The mixture was stirred at 100° C. under N₂ atmosphere overnight. After cooling, the resulting mixture was poured into ice water (10 mL), extracted with EtOAc (10 mL×3), the organic layers were combined and washed with brine (20 mL), dried over anhydrous Na₂SO₄, filtered and concentrated under reduced pressure, and the residue was purified by flash column chromatography on silica gel and prep-TLC to give 5-(cyclopropylmethyl)-6-methyl-2-(2-methyl-2H-indazol-5-yl)-4-(6-methylpyridin-3-yl)-3-oxo-2H,3H,5H-pyrrolopyridazine-7-carbonitrile. ¹H NMR (400 MHz, DMSO-d₆) δ (ppm): 8.52 (d, J=1.9 Hz, 1H), 8.45 (s, 1H), 7.92 (d, J=1.3 Hz, 1H), 7.83 (dd, J=7.9, 2.3 Hz, 1H), 7.66 (d, J=9.2 Hz, 1H), 7.39 (d, J=7.7 Hz, 1H), 7.37 (dd, J=9.2, 1.3 Hz, 1H), 4.20 (s, 3H), 3.56 (dd, J=6.5, 2.2 Hz, 2H), 2.55 (s, 3H), 2.54 (s, 3H), 0.68-0.57 (m, 1H), 0.29-0.21 (m, 2H), −0.02-−0.07 (m, 2H). LC-MS (ESI): m/z 450 [M+H]⁺

### Example 267: Synthesis of 5-(cyclopropylmethyl)-4-(6-cyclopropylpyridin-3-yl)-2-(2-methyl-2H-indazol-5-yl)-7-((methylamino)methyl)-2,5-dihydro-3H-pyrrolo[3,2-c]pyridazin-3-one

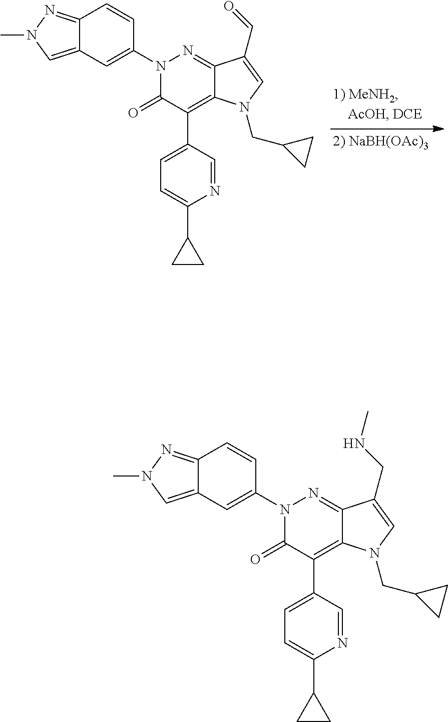 [SMILES: Cn1cc2cc(-n3nc4c(C=O)cn(CC5CC5)c4c(-c4ccc(C5CC5)nc4)c3=O)ccc2n1 ; CNCc1cn(CC2CC2)c2c(-c3ccc(C4CC4)nc3)c(=O)n(-c3ccc4nn(C)cc4c3)nc12]

To a solution of 5-(cyclopropylmethyl)-4-(6-cyclopropylpyridin-3-yl)-2-(2-methyl-2H-indazol-5-yl)-3-oxo-2H,3H,5H-pyrrolo[3,2-c]pyridazine-7-carbaldehyde (100 mg, 0.215 mmol, 1.0 eq.), methanamine (2.0 M in THF) (0.22 mL, 0.44 mmol, 2.0 eq.) and MgSO₄ (259 mg, 2.153 mmol, 10.0 eq.) in DCE (10 mL) was added acetic acid (52 mg, 0.861 mmol, 4.0 eq.) at 0° C. The mixture was stirred at room temperature for 16h. Then NaBH(OAc)₃ (59 mg, 0.280 mmol, 1.3 eq.) was added in one portion at 0° C., and the reaction mixture was allowed to warm to room temperature. The mixture was stirred at room temperature for an additional 2 hrs. The reaction mixture was quenched by adding NaHCO₃(sat. aq.) (5 mL) and extracted with DCM (10 mL×3). The combined organic layers were washed with brine (20 mL), dried over Na₂SO₄, concentrated under reduced pressure, and the residue was purified by flash chromatography and prep-HPLC to afford 5-(cyclopropylmethyl)-4-(6-cyclopropylpyridin-3-yl)-2-(2-methyl-2H-indazol-5-yl)-7-[(methylamino)methyl]-2H,3H,5H-pyrrolo[3,2-c]pyridazin-3-one. ¹H NMR (400 MHz, DMSO-d₆) δ (ppm): 8.47 (d, J=1.8 Hz, 1H), 8.45 (s, 1H), 7.96 (d, J=1.4 Hz, 1H), 7.87 (s, 1H), 7.77 (dd, J=8.0, 2.2 Hz, 1H), 7.65 (d, J=9.2 Hz, 1H), 7.44 (d, J=3.2 Hz, 1H), 7.42 (d, J=2.0 Hz, 1H), 4.21 (s, 3H), 4.02 (s, 2H), 3.36 (s, 2H), 2.52 (s, 3H), 2.22-2.16 (m, 1H), 1.03-0.95 (m, 4H), 0.70-0.58 (m, 1H), 0.36-0.23 (m, 2H), 0.12-0.06 (m, 2H). LC-MS (ESI): m/z 480 [M+H]⁺.

### Example 268: Synthesis of (S)-4-(6-cyclopropylpyridin-3-yl)-5-((2,2-difluorocyclopropyl)methyl)-2-(2-methyl-2H-indazol-5-yl)-3-oxo-3,5-dihydro-2H-pyrrolo[3,2-c]pyridazine-7-carbonitrile

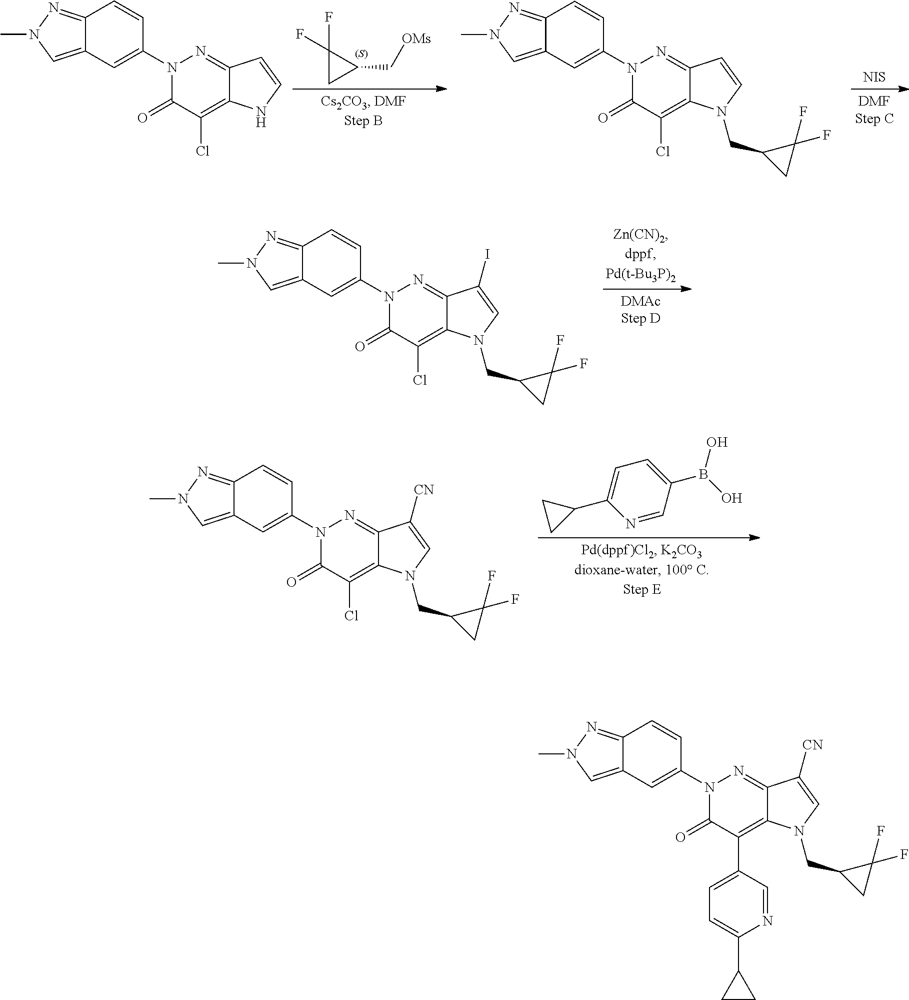

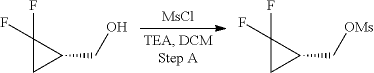 [SMILES: "MsCl" ; OC[C@@H]1CC1(F)F ; CS(=O)(=O)OC[C@@H]1CC1(F)F]

### Step A: (S)-(2,2-difluorocyclopropyl)methyl Methanesulfonate

To solution of (S)-(2,2-difluorocyclopropyl)methanol (167 mg, 1.5 mmol, 1.0 eq.), TEA (312 mg, 3.0 mmol, 2.0 eq.) in THE (5 mL) was added MsCl (354 mg, 3.0 mmol, 2.0 eq.) dropwise at 0° C. After addition, the reaction mixture was allowed to warm to room temperature and stirred for an additional 12 hrs. The reaction was quenched by adding ice water (10 mL), extracted with DCM (15 mL×3), and the organic layers were combined, washed with brine (30 mL), filtered and concentrated under reduced pressure. The resulting residue was purified by flash column chromatography on silica gel to give (S)-(2,2-difluorocyclopropyl)methyl methanesulfonate (210 mg, 75%) as a yellow oil. LC-MS (ESI) m/z 187 [M+H]⁺

### Step B: (S)-4-chloro-5-((2,2-difluorocyclopropyl)methyl)-2-(2-methyl-2H-indazol-5-yl)-2,5-dihydro-3H-pyrrolo[3,2-c]pyridazin-3-one

To a solution of 4-chloro-2-(2-methyl-2H-indazol-5-yl)-2,5-dihydro-3H-pyrrolo[3,2-c]pyridazin-3-one (200 mg, 0.67 mmol, 1.0 eq.) in DMF (6 mL) was added Cs₂CO₃ (542 mg, 1.7 mmol, 2.5 eq.) and (S)-(2,2-difluorocyclopropyl)methyl methanesulfonate (186 mg, 1.0 mmol, 1.5 eq.) at rt. The reaction mixture was stirred at 50° C. for 15 hrs, The resulting mixture was poured into ice water (15 mL), extracted with EtOAc (15 mL×3), the organic layers were combined and washed with brine (20 mL), filtered and concentrated under reduced pressure. The resulting residue was purified by flash column chromatography on silica gel to give (S)-4-chloro-5-((2,2-difluorocyclopropyl)methyl)-2-(2-methyl-2H-indazol-5-yl)-2,5-dihydro-3H-pyrrolo[3,2-c]pyridazin-3-one (167 mg, 62%) as a yellow solid. LC-MS (ESI): m/z 390 [M+H]⁺.

### Step C: (S)-4-chloro-5-((2,2-difluorocyclopropyl)methyl)-7-iodo-2-(2-methyl-2H-indazol-5-yl)-2,5-dihydro-3H-pyrrolo[3,2-c]pyridazin-3-one

To a solution of (S)-4-chloro-5-((2,2-difluorocyclopropyl)methyl)-2-(2-methyl-2H-indazol-5-yl)-2,5-dihydro-3H-pyrrolo[3,2-c]pyridazin-3-one (167 mg, 0.42 mmol, 1.0 eq.) in DMF (5 mL) was added NIS (124 mg, 0.55 mmol, 1.3 eq.) at room temperature. The reaction mixture was stirred at 85° C. for 2 hrs under N₂ atmosphere. The resulting mixture was poured into ice water (15 mL) and extracted with EtOAc (15 mL×3). The organic layers were combined and washed with brine (20 mL), filtered and concentrated under reduced pressure, and the residue was purified by flash column chromatography on silica gel to give (S)-4-chloro-5-((2,2-difluorocyclopropyl)methyl)-7-iodo-2-(2-methyl-2H-indazol-5-yl)-2,5-dihydro-3H-pyrrolo[3,2-c]pyridazin-3-one (150 mg, 68%) as a yellow solid. LC-MS (ESI): m/z 516 [M+H]⁺.

### Step D: 7-chloro-1-(((S)-2,2-difluorocyclopropyl)methyl)-5-(2-methyl-2H-indazol-5-yl)-6-oxo-5,6-dihydro-1H-pyrrolo[3,2-b]pyridine-3-carbonitrile

To a solution of (S)-4-chloro-5-((2,2-difluorocyclopropyl)methyl)-7-iodo-2-(2-methyl-2H-indazol-5-yl)-2,5-dihydro-3H-pyrrolo[3,2-c]pyridazin-3-one (150 mg, 0.29 mmol, 1.0 eq.), Zn(CN)₂ (130 mg, 1.16 mmol, 4.0 eq.) and dppf (16 mg, 0.029 mmol, 0.1 eq.) in DMAc (8 mL) was added Pd(tBu₃)₂ (14 mg, 0.029 mmol, 0.1 eq.) at room temperature. The reaction mixture was kept stirring at 85° C. for 2 hrs under N₂ atmosphere. The resulting mixture was poured into ice water (15 mL) and extracted with EtOAc (15 mL×3). The organic layers were combined and washed with brine (20 mL), filtered, concentrated under reduced pressure, and the resulting residue was purified by flash column chromatography on silica gel to give 7-chloro-1-(((S)-2,2-difluorocyclopropyl)methyl)-5-(2-methyl-2H-indazol-5-yl)-6-oxo-5,6-dihydro-1H-pyrrolo[3,2-b]pyridine-3-carbonitrile (90 mg, 75%) as a yellow solid. LC-MS (ESI): m/z 415 [M+H]⁺.

### Step E: (S)-4-(6-cyclopropylpyridin-3-yl)-5-((2,2-difluorocyclopropyl)methyl)-2-(2-methyl-2H-indazol-5-yl)-3-oxo-3,5-dihydro-2H-pyrrolo[3,2-c]pyridazine-7-carbonitrile

To a solution of (6-cyclopropylpyridin-3-yl)boronic acid (43 mg, 0.26 mmol, 2.2 eq.) and 7-chloro-1-(((S)-2,2-difluorocyclopropyl)methyl)-5-(2-methyl-2H-indazol-5-yl)-6-oxo-5,6-dihydro-1H-pyrrolo[3,2-b]pyridine-3-carbonitrile (50 mg, 0.12 mmol, 1.0 eq.) in dioxane/H₂O (5 mL, 4:1, v:v) was added K₂CO₃ (41 mg, 0.301 mmol, 2.5 eq.) and Pd(dppf)Cl₂ (8 mg, 0.01 mmol, 0.1 eq.) at room temperature. The reaction mixture was stirred at 100° C. for 5 hrs under N₂ atmosphere. The resulting mixture was poured into ice water (15 mL) and extracted with EA (15 mL×3). The organic layers were combined, washed with brine (20 mL), filtered and concentrated under reduced pressure. The resulting residue was purified by flash column chromatography on silica gel to give (S)-4-(6-cyclopropylpyridin-3-yl)-5-((2,2-difluorocyclopropyl)methyl)-2-(2-methyl-2H-indazol-5-yl)-3-oxo-3,5-dihydro-2H-pyrrolo[3,2-c]pyridazine-7-carbonitrile. ¹H NMR (400 MHz, DMSO-d₆) S(ppm): 8.68 (s, 1H), 8.52 (d, J=2.0 Hz, 1H), 8.47 (s, 1H), 7.94 (d, J=1.6 Hz, 1H), 7.82 (dd, J=8.0, 2.0 Hz, 1H), 7.68 (d, J=9.2 Hz, 1H), 7.45 (d, J=8.0 Hz, 1H), 7.38 (dd, J=9.2, 2.0 Hz, 1H), 4.21 (s, 3H), 3.80-3.75 (m, 1H), 3.59-3.54 (m, 1H), 2.21-2.15 (m, 1H), 1.80-1.65 (m, 1H), 1.60-1.49 (m, 1H), 1.35-1.28 (m, 1H), 1.05-0.95 (m, 4H). LC-MS (ESI): m/z 498 [M+H]⁺.

### Example 269: Preparation of (R)-4-(6-cyclopropylpyridin-3-yl)-5-((2,2-difluorocyclopropyl)methyl)-2-(2-methyl-2H-indazol-5-yl)-3-oxo-3,5-dihydro-2H-pyrrolo[3,2-c]pyridazine-7-carbonitrile

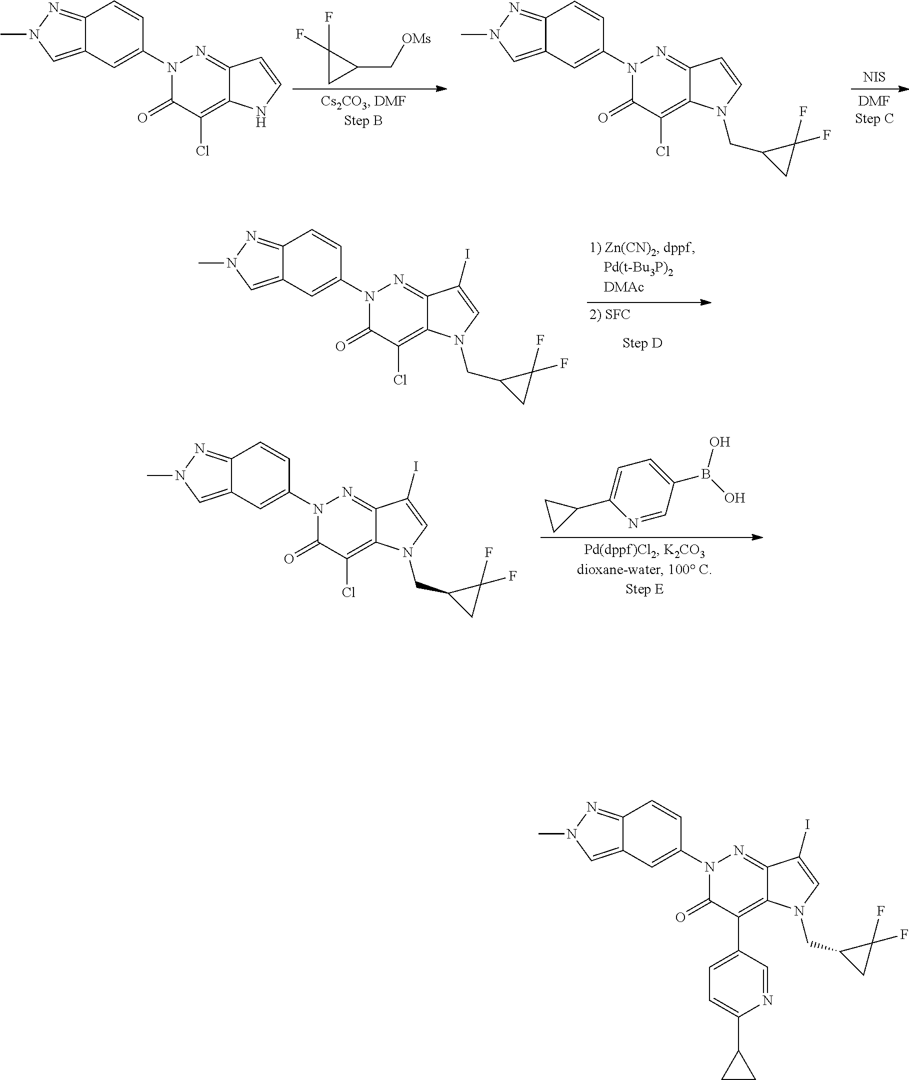

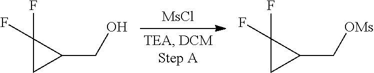 [SMILES: "MsCl" ; OCC1CC1(F)F ; CS(=O)(=O)OCC1CC1(F)F]

### Step A: (rac)-(2,2-difluorocyclopropyl)methyl methanesulfonate

To solution of rac-(2,2-difluorocyclopropyl)methanol (167 mg, 1.5 mmol, 1.0 eq.), TEA (312 mg, 3.0 mmol, 2.0 eq.) in THE (5 mL) was added MsCl (354 mg, 3.0 mmol, 2.0 eq) dropwise at 0° C. After addition, the reaction mixture was allowed to warm to room temperature and stirred for an additional 12 hrs. The reaction was quenched by adding ice water (10 mL) and extracted with DCM (15 mL×3). The organic layers was combined, washed with brine (30 mL), filtered and concentrated under reduced pressure. The resulting residue was purified by flash column chromatography on silica gel to give (rac)-(2,2-difluorocyclopropyl)methyl methanesulfonate (220 mg, 79%) as a yellow oil. LC-MS (ESI) m/z 187 [M+H]⁺.

### Step B: (rac)-4-chloro-5-((2,2-difluorocyclopropyl)methyl)-2-(2-methyl-2H-indazol-5-yl)-2,5-dihydro-3H-pyrrolo[3,2-c]pyridazin-3-one

To a solution of 4-chloro-2-(2-methyl-2H-indazol-5-yl)-2,5-dihydro-3H-pyrrolo[3,2-c]pyridazin-3-one (200 mg, 0.67 mmol, 1.0 eq.) in DMF (6 mL) was added Cs₂CO₃ (542 mg, 1.7 mmol, 2.5 eq.) and (rac)-(2,2-difluorocyclopropyl)methyl methanesulfonate (186 mg, 1.0 mmol, 1.5 eq) at rt. The reaction mixture was stirred at 50° C. for 15 hrs. The resulting mixture was poured into ice water (15 mL) and extracted with EA (15 mL×3). The organic layers were combined and washed with brine (20 mL), filtered and concentrated under reduced pressure. The resulting residue was purified by flash column chromatography on silica gel to give (rac)-4-chloro-5-((2,2-difluorocyclopropyl)methyl)-2-(2-methyl-2H-indazol-5-yl)-2,5-dihydro-3H-pyrrolo[3,2-c]pyridazin-3-one (200 mg, 79%) as a yellow solid. LC-MS (ESI): m/z 390 [M+H]⁺.

### Step C: 4-chloro-5-((2,2-difluorocyclopropyl)methyl)-7-iodo-2-(2-methyl-2H-indazol-5-yl)-2,5-dihydro-3H-pyrrolo[3,2-c]pyridazin-3-one

To a solution of (rac)-4-chloro-5-((2,2-difluorocyclopropyl)methyl)-2-(2-methyl-2H-indazol-5-yl)-2,5-dihydro-3H-pyrrolo[3,2-c]pyridazin-3-one (200 mg, 0.51 mmol, 1.0 eq.) in DMF (5 mL) was added NIS (149 mg, 0.67 mmol, 1.3 eq.) at room temperature. The reaction mixture was stirred at 85° C. for 2 hrs under N₂ atmosphere. The resulting mixture was poured into ice water (15 mL) and extracted with EA (15 mL×3). The organic layers were combined, washed with brine (20 mL), filtered and concentrated under reduced pressure. The resulting residue was purified by flash column chromatography on silica gel to give (rac)-4-chloro-5-((2,2-difluorocyclopropyl)methyl)-7-iodo-2-(2-methyl-2H-indazol-5-yl)-2,5-dihydro-3H-pyrrolo[3,2-c]pyridazin-3-one (180 mg, 68%) as a yellow solid. LC-MS (ESI): m/z 516 [M+H]⁺.

### Step D: 7-chloro-1-(((R)-2,2-difluorocyclopropyl)methyl)-5-(2-methyl-2H-indazol-5-yl)-6-oxo-5,6-dihydro-1H-pyrrolo[3,2-b]pyridine-3-carbonitrile

To a solution of (rac)-4-chloro-5-((2,2-difluorocyclopropyl)methyl)-7-iodo-2-(2-methyl-2H-indazol-5-yl)-2,5-dihydro-3H-pyrrolo[3,2-c]pyridazin-3-one (150 mg, 0.29 mmol, 1.0 eq.), Zn(CN)₂(130 mg, 1.16 mmol, 4.0 eq.) and dppf (16 mg, 0.029 mmol, 0.1 eq.) in DMAc (8 mL) was added Pd(tBu₃)₂ (14 mg, 0.029 mmol, 0.1 eq.) at room temperature. The reaction mixture was stirred at 85° C. for 2 hrs under N₂ atmosphere. The resulting mixture was poured into ice water (15 mL) and extracted with EA (15 mL×3). The organic layers were combined, washed with brine (20 mL), filtered and concentrated under reduced pressure. The resulting residue was purified by flash column chromatography on silica gel to give (rac)-7-chloro-1-(((S)-2,2-difluorocyclopropyl)methyl)-5-(2-methyl-2H-indazol-5-yl)-6-oxo-5,6-dihydro-1H-pyrrolo[3,2-b]pyridine-3-carbonitrile, then separated by chiral SFC to give 7-chloro-1-(((R)-2,2-difluorocyclopropyl)methyl)-5-(2-methyl-2H-indazol-5-yl)-6-oxo-5,6-dihydro-1H-pyrrolo[3,2-b]pyridine-3-carbonitrile (40 mg, 33%) as a yellow solid. LC-MS (ESI): m/z 415 [M+H]⁺.

### Step E: (R)-4-(6-cyclopropylpyridin-3-yl)-5-((2,2-difluorocyclopropyl)methyl)-2-(2-methyl-2H-indazol-5-yl)-3-oxo-3,5-dihydro-2H-pyrrolo[3,2-c]pyridazine-7-carbonitrile

To a solution of (6-cyclopropylpyridin-3-yl)boronic acid (43 mg, 0.26 mmol, 2.2 eq.) and 7-chloro-1-(((R)-2,2-difluorocyclopropyl)methyl)-5-(2-methyl-2H-indazol-5-yl)-6-oxo-5,6-dihydro-1H-pyrrolo[3,2-b]pyridine-3-carbonitrile (50 mg, 0.12 mmol, 1.0 eq.) in dioxane/H₂O (5 mL, 4:1, v:v) was added K₂CO₃ (41 mg, 0.301 mmol, 2.5 eq.) and Pd(dppf)Cl₂ (8 mg, 0.01 mmol, 0.1 eq.) at room temperature. The reaction mixture was kept stirring at 100° C. for 5 hrs under N₂ atmosphere. The resulting mixture was poured into ice water (15 mL) and extracted with EA (15 mL×3). The organic layers were combined, washed with brine (20 mL), filtered and concentrated under reduced pressure. The resulting residue was purified by flash column chromatography on silica gel to give (R)-4-(6-cyclopropylpyridin-3-yl)-5-((2,2-difluorocyclopropyl)methyl)-2-(2-methyl-2H-indazol-5-yl)-3-oxo-3,5-dihydro-2H-pyrrolo[3,2-c]pyridazine-7-carbonitrile. ¹H NMR (400 MHz, DMSO-d₆) δ (ppm): 8.68 (s, 1H), 8.52 (d, J=2.0 Hz, 1H), 8.47 (s, 1H), 7.94 (d, J=1.6 Hz, 1H), 7.82 (dd, J=8.0, 2.0 Hz, 1H), 7.68 (d, J=9.2 Hz, 1H), 7.45 (d, J=8.0 Hz, 1H), 7.38 (dd, J=9.2, 2.0 Hz, 1H), 4.21 (s, 3H), 3.80-3.75 (m, 1H), 3.3.59-3.54 (m, 1H), 2.21-2.15 (m, 1H), 1.80-1.65 (m, 1H), 1.60-1.49 (m, 1H), 1.42-1.28 (s, 1H), 1.02-0.97 (m, 4H). LC-MS (ESI): m/z 498 [M+H]⁺

### Example 270: Synthesis of 5-(cyclopropylmethyl)-4-(6-cyclopropylpyridin-3-yl)-2-(2-(methyl-d3)-2H-indazol-5-yl)-3-oxo-3,5-dihydro-2H-pyrrolo[3,2-c]pyridazine-7-carbonitrile

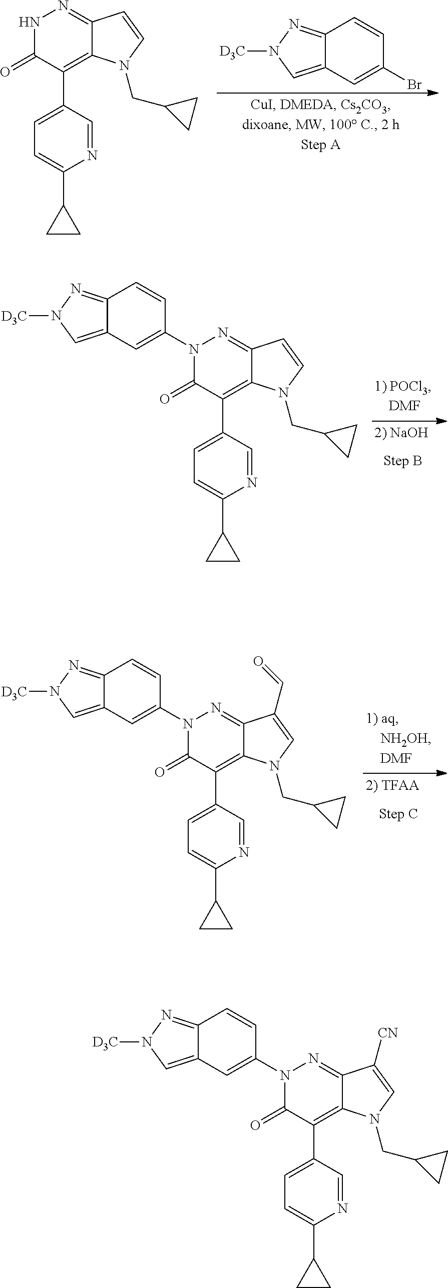 [SMILES: O=c1[nH]nc2ccn(CC3CC3)c2c1-c1ccc(C2CC2)nc1 ; [2H]C([2H])([2H])n1cc2cc(Br)ccc2n1 ; [2H]C([2H])([2H])n1cc2cc(-n3nc4ccn(CC5CC5)c4c(-c4ccc(C5CC5)nc4)c3=O)ccc2n1 ; [2H]C([2H])([2H])n1cc2cc(-n3nc4c(C=O)cn(CC5CC5)c4c(-c4ccc(C5CC5)nc4)c3=O)ccc2n1 ; [2H]C([2H])([2H])n1cc2cc(-n3nc4c(C#N)cn(CC5CC5)c4c(-c4ccc(C5CC5)nc4)c3=O)ccc2n1]

### Step A: 5-(cyclopropylmethyl)-4-(6-cyclopropylpyridin-3-yl)-2-(2-(methyl-d₃)-2H-indazol-5-yl)-2,5-dihydro-3H-pyrrolo[3,2-c]pyridazin-3-one

To a solution of 5-(cyclopropylmethyl)-4-(6-cyclopropylpyridin-3-yl)-2,5-dihydro-3H-pyrrolo[3,2-c]pyridazin-3-one (240 mg, 0.783 mmol, 1.0 eq.) in dioxane (2 mL) was added Cs₂CO₃ (766 mg, 2.35 mmol, 3.0 eq.), N₁,N₂-dimethylethane-1,2-diamine (138 mg, 1.57 mmol, 2.0 eq.), 5-bromo-2-(methyl-d3)-2H-indazole (201 mg, 0.94 mmol, 1.2 eq.) (which synthesized from 5-bromo-2H-indazole and iodomethane-d3 according to WO-2016/180536) and CuI (149 mg, 0.78 mmol, 1.0 eq.). The reaction mixture was degassed with N₂, sealed in a tube and heated to 100° C. in a microwave for 2.0 hrs. The reaction was quenched with conc. NH₄₀H (1 mL) and the aqueous layer was extracted with DCM (20 mL×3). The combined organic phases were dried over anhydrous Na₂SO₄, concentrated under reduced pressure, and the residue was purified by flash column chromatography on silica gel to give 5-(cyclopropylmethyl)-4-(6-cyclopropylpyridin-3-yl)-2-(2-(methyl-d3)-2H-indazol-5-yl)-2,5-dihydro-3H-pyrrolo[3,2-c]pyridazin-3-one (180 mg, 52%) as a yellow solid. LC-MS (ESI): m/z 440 [M+H]⁺.

### Step B: 5-(cyclopropylmethyl)-4-(6-cyclopropylpyridin-3-yl)-2-(2-(methyl-d3)-2H-indazol-5-yl)-3-oxo-3,5-dihydro-2H-pyrrolo[3,2-c]pyridazine-7-carbaldehyde

To a solution of 5-(cyclopropylmethyl)-4-(6-cyclopropylpyridin-3-yl)-2-(2-(methyl-d3)-2H-indazol-5-yl)-2,5-dihydro-3H-pyrrolo[3,2-c]pyridazin-3-one (363 mg, 0.83 mmol, 1.0 eq.) in dry DMF (4 mL) was added POCl₃ (510 mg, 3.33 mmol, 4.0 eq.) at 0° C. The reaction mixture was stirred at room temperature for an additional 15 hrs, then NaOH (1N, aq.) (10 mL) was added into the mixture at 0° C. The reaction mixture was stirred at rt for 3 hrs until LCMS analysis showed the reaction had completed. The resulting mixture was poured into ice-cooled NaHCO₃(sat. aq.) (15 mL), extracted with DCM (20 mL×3), washed with brine (20 mL), dried over Na₂SO₄, concentrated under reduced pressure, and the residue was purified by flash column chromatography on silica gel to give 5-(cyclopropylmethyl)-4-(6-cyclopropylpyridin-3-yl)-2-(2-(methyl-d3)-2H-indazol-5-yl)-3-oxo-3,5-dihydro-2H-pyrrolo[3,2-c]pyridazine-7-carbaldehyde (330 mg, 85%) as a yellow solid. LC-MS (ESI): m/z 468 [M+H]⁺.

### Step C: 5-(cyclopropylmethyl)-4-(6-cyclopropylpyridin-3-yl)-2-(2-(methyl-d3)-2H-indazol-5-yl)-3-oxo-3,5-dihydro-2H-pyrrolo[3,2-c]pyridazine-7-carbonitrile

To a solution of 5-(cyclopropylmethyl)-4-(6-cyclopropylpyridin-3-yl)-2-(2-(methyl-d3)-2H-indazol-5-yl)-3-oxo-3,5-dihydro-2H-pyrrolo[3,2-c]pyridazine-7-carbaldehyde (200 mg, 0.43 mmol, 1.0 eq.) in DMF (5 mL), was added NH₂OH (50% aq.) (0.5 mL). The resulting mixture was stirred at 40° C. for 2 hrs and the reaction progress was monitored by TLC. Once TLC indicated full consumption of the aldehyde, the mixture was cooled to 0° C. and TFAA (240 μL, 1.72 mmol, 4.0 eq.) was added. The reaction mixture was then stirred at rt for an additional 15 hrs, until LCMS showed complete conversion. The reaction was quenched with ice-cooled NaHCO₃(sat. aq.) (10 mL), extracted with EtOAc (10 mL×3), washed with brine (20 mL), dried over Na₂SO₄, and concentrated under reduced pressure. The resulting residue was purified by flash column chromatography on silica gel to give 5-(cyclopropylmethyl)-4-(6-cyclopropylpyridin-3-yl)-2-(2-(methyl-d3)-2H-indazol-5-yl)-3-oxo-3,5-dihydro-2H-pyrrolo[3,2-c]pyridazine-7-carbonitrile. ¹H NMR (400 MHz, DMSO-d₆) δ (ppm): 8.71 (s, 1H), 8.50 (d, J=1.7 Hz, 1H), 8.46 (d, J=0.8 Hz, 1H), 7.97-7.89 (m, 1H), 7.80 (dd, J=8.0, 2.3 Hz, 1H), 7.68 (d, J=9.1 Hz, 1H), 7.44 (d, J=7.5 Hz, 1H), 7.38 (dd, J=9.2, 2.0 Hz, 1H), 3.39 (d, J=7.2 Hz, 2H), 2.27-2.13 (m, 1H), 1.02-0.96 (m, 4H), 0.73-0.69 (m, 1H), 0.40-0.31 (m, 2H), 0.19-0.11 (m, 2H). LC-MS (ESI): m/z 465 [M+H]⁺.

### Example 271 & 272: 5-(cyclopropylmethyl)-4-(4-(difluoromethoxy)phenyl)-2-(2-methyl-2H-indazol-5-yl)-2,7-dihydro-3H-pyrrolo[2,3-c]pyridazin-3-one (Example 271) and 5-(cyclopropylmethyl)-4-(4-(difluoromethoxy)phenyl)-7-methyl-2-(2-methyl-2H-indazol-5-yl)-2,7-dihydro-3H-pyrrolo[2,3-c]pyridazin-3-one (Example 272)

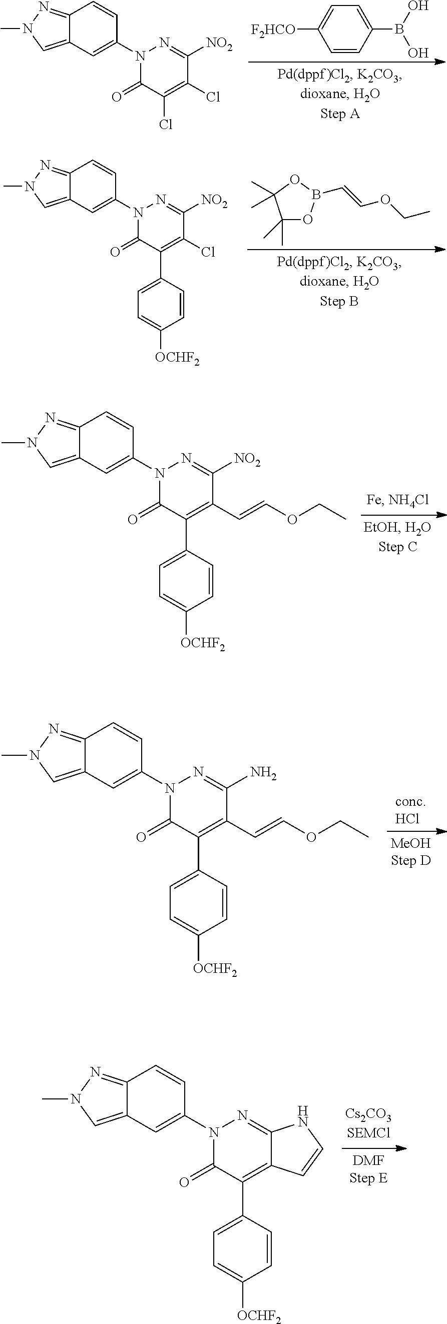

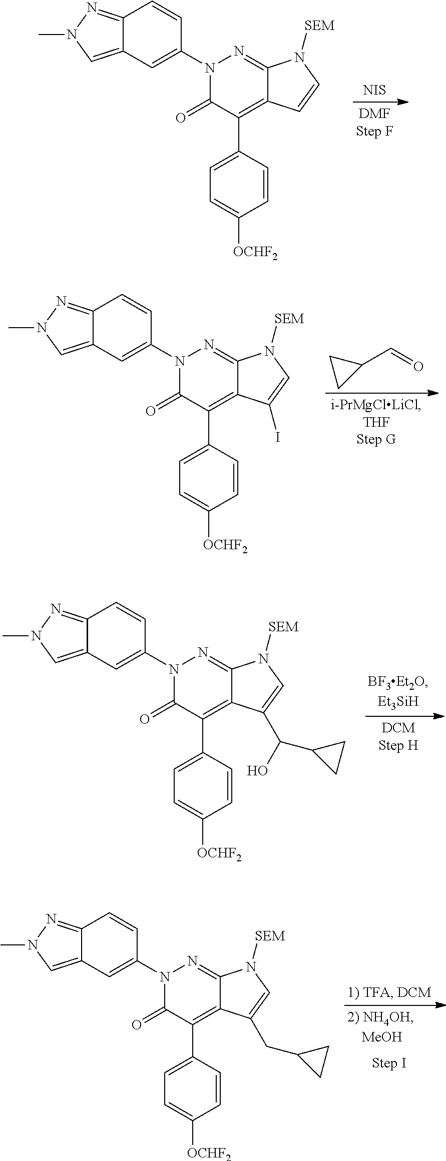

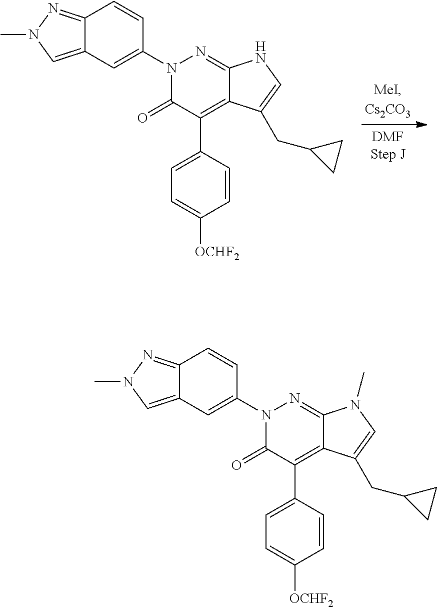 [SMILES: Cn1cc2cc(-n3nc4[nH]cc(CC5CC5)c4c(-c4ccc(OC(F)F)cc4)c3=O)ccc2n1 ; Cn1cc2cc(-n3nc4c(c(CC5CC5)cn4C)c(-c4ccc(OC(F)F)cc4)c3=O)ccc2n1]

### Step A: 5-chloro-4-(4-(difluoromethoxy)phenyl)-2-(2-methyl-2H-indazol-5-yl)-6-nitropyridazin-3(2H)-one

A solution of 4,5-dichloro-2-(2-methyl-2H-indazol-5-yl)-6-nitro-2,3-dihydropyridazin-3-one(4 g, 11.76 mmol, 1.0 eq.), [4-(difluoromethoxy)phenyl]boronic acid (2.43 g, 12.936 mmol, 1.1 eq.), Pd(dppf)Cl₂ (0.43 g, 0.588 mmol, 0.05 eq.) and K₂CO₃ (3.25 g, 23.520 mmol, 2.0 eq.) in dioxane/water (60 mL, 4:1, v/v) was stirred at 80° C. for 2 hrs under N₂ atmosphere. After completion, the reaction mixture was filtered through a short pad of Celite®, the filtrate was concentrated under reduced pressure, and the residue was purified by flash column chromatography on silica gel to give 5-chloro-4-(4-(difluoromethoxy)phenyl)-2-(2-methyl-2H-indazol-5-yl)-6-nitropyridazin-3(2H)-one (1.3 g, 82%) as a yellow solid. LC-MS (ESI): m/z 448 [M+H]⁺.

### Step B: (E)-4-(4-(difluoromethoxy)phenyl)-5-(2-ethoxyvinyl)-2-(2-methyl-2H-indazol-5-yl)-6-nitropyridazin-3(2H)-one

A solution of 5-chloro-4-[4-(difluoromethoxy)phenyl]-2-(2-methyl-2H-indazol-5-yl)-6-nitro-2,3-dihydropyridazin-3-one (4.3 g, 9.603 mmol, 1.0 eq.), 2-[(E)-2-ethoxyethenyl]-4,4,5,5-tetramethyl-1,3,2-dioxaborolane (2.441 mL, 11.523 mmol, 1.2 eq.), Pd(dppf)Cl₂ (0.35 g, 0.480 mmol, 0.05 eq.) and K₂CO₃ (2.65 g, 19.205 mmol, 2.0 eq.) in dioxane/water (60 mL, 4:1, v/v) was stirred was stirred at 80° C. for 2 hrs under N₂ atmosphere. After completion, the reaction mixture was filtered through a short pad of Celite®, the filtrate was concentrated under reduced pressure, and the residue was purified by flash column chromatography on silica gel to give (E)-4-(4-(difluoromethoxy)phenyl)-5-(2-ethoxyvinyl)-2-(2-methyl-2H-indazol-5-yl)-6-nitropyridazin-3(2H)-one (4.2 g, 91%) as a yellow solid. LC-MS (ESI): m/z 484 [M+H]⁺.

### Step C: (E)-6-amino-4-(4-(difluoromethoxy)phenyl)-5-(2-ethoxyvinyl)-2-(2-methyl-2H-indazol-5-yl)pyridazin-3(2H)-one

To a solution of (E)-4-(4-(difluoromethoxy)phenyl)-5-(2-ethoxyvinyl)-2-(2-methyl-2H-indazol-5-yl)-6-nitropyridazin-3(2H)-one (4.2 g, 8.688 mmol, 1.0 eq.) in EtOH (60 mL) and H₂O (15 mL) was added Fe⁰ (487 mg, 8.688 mmol, 1.0 eq.) and NH₄Cl (460 mg, 8.688 mmol, 1.0 eq.). The reaction mixture was stirred at 40° C. for 12 hrs. After completion, the reaction mixture was filtered through a short pad of Celite®, the filtrate was concentrated under reduced pressure, and the residue was purified by flash column chromatography on silica gel to give (E)-6-amino-4-(4-(difluoromethoxy)phenyl)-5-(2-ethoxyvinyl)-2-(2-methyl-2H-indazol-5-yl)pyridazin-3(2H)-one (2.3 g, 58%) as a yellow solid. LC-MS (ESI): m/z 454 [M+H]⁺.

### Step D: 4-(4-(difluoromethoxy)phenyl)-2-(2-methyl-2H-indazol-5-yl)-2,7-dihydro-3H-pyrrolo[2,3-c]pyridazin-3-one

A solution of (E)-6-amino-4-(4-(difluoromethoxy)phenyl)-5-(2-ethoxyvinyl)-2-(2-methyl-2H-indazol-5-yl)pyridazin-3(2H)-one (2.3 g, 5.072 mmol, 1.0 eq.) in MeOH (50 mL) and conc. HCl (0.5 mL) was stirred at 80° C. for 3 hrs. After completion, the reaction mixture was concentrated under reduced pressure, the residue was diluted with DCM (100 mL), washed with aq. NaHCO₃(40 mL) and brine (20 mL), dried over anhydrous Na₂SO₄, concentrated under reduced pressure, and the residue was purified was purified by flash column chromatography on silica gel to give 4-(4-(difluoromethoxy)phenyl)-2-(2-methyl-2H-indazol-5-yl)-2,7-dihydro-3H-pyrrolo[2,3-c]pyridazin-3-one (720 mg, 35%) as a yellow solid. LC-MS (ESI): m/z 408 [M+H]⁺.

### Step E: 4-(4-(difluoromethoxy)phenyl)-2-(2-methyl-2H-indazol-5-yl)-7-((2-(trimethylsilyl)ethoxy)methyl)-2,7-dihydro-3H-pyrrolo[2,3-c]pyridazin-3-one

To a solution of 4-[4-(difluoromethoxy)phenyl]-2-(2-methyl-2H-indazol-5-yl)-2H,3H,7H-pyrrolo[2,3-c]pyridazin-3-one (407 mg, 1.0 mmol, 1.0 eq.) in DMF (4 mL) was added Cs₂CO₃ (651 mg, 2.0 mmol, 2.0 eq.) and SEMCl (0.2 mL, 1.3 mmol, 1.3 eq.) at 0° C. The reaction mixture was stirred at room temperature for 1 hr. After completion, the reaction mixture was diluted with H₂O (20 mL) and extracted with DCM (50 mL×2). The combined organic layers were washed with brine (40 mL), dried over Na₂SO₄, concentrated under reduced pressure, and the residue was purified by flash column chromatography on silica gel to give 4-(4-(difluoromethoxy)phenyl)-2-(2-methyl-2H-indazol-5-yl)-7-((2-(trimethylsilyl)ethoxy)methyl)-2,7-dihydro-3H-pyrrolo[2,3-c]pyridazin-3-one (431 mg, 80%) as a yellow solid. LC-MS (ESI): m/z 538 [M+H]⁺.

### Step F: 4-(4-(difluoromethoxy)phenyl)-5-iodo-2-(2-methyl-2H-indazol-5-yl)-7-((2-(trimethylsilyl)ethoxy)methyl)-2,7-dihydro-3H-pyrrolo[2,3-c]pyridazin-3-one

To a solution of 4-(4-(difluoromethoxy)phenyl)-2-(2-methyl-2H-indazol-5-yl)-7-((2-(trimethylsilyl)ethoxy)methyl)-2,7-dihydro-3H-pyrrolo[2,3-c]pyridazin-3-one (200 mg, 0.37 mmol, 1.0 eq.) in DMF (2 mL) was added NIS (109 mg, 0.48 mmol, 1.3 eq.). The reaction mixture was stirred at 85° C. under N₂ for 2 hrs. After completion, the reaction mixture was quenched with aq. Na₂S203 (15 mL) and extracted with DCM (30 mL×2). The combined organic layers were washed with brine (20 mL), dried over Na₂SO₄, concentrated under reduced pressure, and the residue was purified by flash column chromatography on silica gel to give 4-(4-(difluoromethoxy)phenyl)-5-iodo-2-(2-methyl-2H-indazol-5-yl)-7-((2-(trimethylsilyl)ethoxy)methyl)-2,7-dihydro-3H-pyrrolo[2,3-c]pyridazin-3-one (230 mg, 93%) as a yellow solid. LC-MS (ESI): m/z 664 [M+H]⁺.

### Step G: 5-(cyclopropyl(hydroxy)methyl)-4-(4-(difluoromethoxy)phenyl)-2-(2-methyl-2H-indazol-5-yl)-7-((2-(trimethylsilyl)ethoxy)methyl)-2,7-dihydro-3H-pyrrolo[2,3-c]pyridazin-3-one

To a solution of 4-(4-(difluoromethoxy)phenyl)-5-iodo-2-(2-methyl-2H-indazol-5-yl)-7-((2-(trimethylsilyl)ethoxy)methyl)-2,7-dihydro-3H-pyrrolo[2,3-c]pyridazin-3-one (355 mg, 0.54 mmol, 1.0 eq.) in THE (2 mL) was added isopropylmagnesium chloride-lithium chloride complex solution (3 mL, 3.9 mmol, 7.3 eq., 1.3 M in THF) dropwise at −78° C. The reaction mixture was stirred at −78° C. under N₂ atmosphere for an additional 1 hr, then cyclopropanecarbaldehyde (1 mL, 13.4 mmol, 24.8 eq.) was added dropwise, and the reaction mixture was stirred at −78° C. for an additional 0.5 h. After completion, the reaction mixture was quenched with NH₄Cl (sat. aq.) (40 mL), thawed to room temperature, and extracted with DCM (50 mL×2). The combined organic layers were washed with brine (10 mL), dried over Na₂SO₄, concentrated under reduced pressure, and the residue was purified by flash column chromatography on silica gel to give 5-(cyclopropyl(hydroxy)methyl)-4-(4-(difluoromethoxy)phenyl)-2-(2-methyl-2H-indazol-5-yl)-7-((2-(trimethylsilyl)ethoxy)methyl)-2,7-dihydro-3H-pyrrolo[2,3-c]pyridazin-3-one (40 mg, 12%) as a yellow solid. LC-MS (ESI): m/z 608 [M+H]⁺.

### Step H: 5-(cyclopropylmethyl)-4-(4-(difluoromethoxy)phenyl)-2-(2-methyl-2H-indazol-5-yl)-7-((2-(trimethylsilyl)ethoxy)methyl)-2,7-dihydro-3H-pyrrolo[2,3-c]pyridazin-3-one

To a solution of 5-(cyclopropyl(hydroxy)methyl)-4-(4-(difluoromethoxy)phenyl)-2-(2-methyl-2H-indazol-5-yl)-7-((2-(trimethylsilyl)ethoxy)methyl)-2,7-dihydro-3H-pyrrolo[2,3-c]pyridazin-3-one (40 mg, 0.066 mmol, 1.0 eq.) in DCM (2 mL) was added triethylsilane (0.053 mL, 0.330 mmol, 5.0 eq.) and BF₃.Et₂O (28 mg, 0.198 mmol, 3.0 eq.) at −78° C. The reaction mixture was stirred at −78° C. under N₂ atmosphere for 0.5 h, then the reaction mixture was quenched with NaHCO₃(sat. aq.) (15 mL), thawed to room temperature, and extracted with DCM (30 mL×2). The combined organic layers were washed with brine (10 mL), dried over Na₂SO₄, and concentrated under reduced pressure to afford 5-(cyclopropylmethyl)-4-(4-(difluoromethoxy)phenyl)-2-(2-methyl-2H-indazol-5-yl)-7-((2-(trimethylsilyl)ethoxy)methyl)-2,7-dihydro-3H-pyrrolo[2,3-c]pyridazin-3-one (30 mg, crude) as a yellow solid, which was taken on to the next step directly. LC-MS (ESI): m/z 592 [M+H]⁺.

### Step I: 5-(cyclopropylmethyl)-4-(4-(difluoromethoxy)phenyl)-2-(2-methyl-2H-indazol-5-yl)-2,7-dihydro-3H-pyrrolo[2,3-c]pyridazin-3-one

To a solution of crude 5-(cyclopropylmethyl)-4-(4-(difluoromethoxy)phenyl)-2-(2-methyl-2H-indazol-5-yl)-7-((2-(trimethylsilyl)ethoxy)methyl)-2,7-dihydro-3H-pyrrolo[2,3-c]pyridazin-3-one (30 mg, 0.053 mmol, 1.0 eq.) in DCM (1 mL) was added TFA (0.1 mL), and the reaction mixture was stirred at room temperature for 2 hours. The reaction mixture was then concentrated under reduced pressure and the residue was retaken in a mixture of MeOH (0.5 mL) and THE (0.5 mL). Conc. NH₄₀H (1 mL) was added and the reaction mixture was stirred at room temperature for additional 1 hr. After completion, the reaction mixture was diluted with H₂O (20 mL), extracted with DCM (30 mL×2), the combined organic layers were washed with brine (10 mL), dried over Na₂SO₄, concentrated under reduced pressure, and the residue was purified by Prep-TLC to afford 5-(cyclopropylmethyl)-4-(4-(difluoromethoxy)phenyl)-2-(2-methyl-2H-indazol-5-yl)-2,7-dihydro-3H-pyrrolo[2,3-c]pyridazin-3-one (Example 271). ¹H NMR (400 MHz, DMSO-d₆) δ (ppm): 11.12 (s, 1H), 8.42 (s, 1H), 7.90 (d, J=1.5 Hz, 1H), 7.67-7.61 (m, 2H), 7.49 (d, J=8.5 Hz, 2H), 7.39 (dd, J=9.2 Hz, 2.0 Hz, 1H), 7.32 (t, J_{HF}=74 Hz, 1H), 7.24 (d, J=8.6 Hz, 2H), 4.20 (s, 3H), 1.96 (d, J=6.7 Hz, 2H), 0.49-0.44 (m, 1H), 0.28-0.23 (m, 2H), −0.14-−0.1 (m, 2H). LC-MS (ESI): m/z 462 [M+H]⁺.

### Step F: 5-(cyclopropylmethyl)-4-(4-(difluoromethoxy)phenyl)-7-methyl-2-(2-methyl-2H-indazol-5-yl)-2,7-dihydro-3H-pyrrolo[2,3-c]pyridazin-3-one

To a solution of 5-(cyclopropylmethyl)-4-(4-(difluoromethoxy)phenyl)-2-(2-methyl-2H-indazol-5-yl)-2,7-dihydro-3H-pyrrolo[2,3-c]pyridazin-3-one (20 mg, 0.049 mmol, 1.0 eq.) in DMF (1 mL) was added Mel (9 mg, 0.064 mmol, 1.3 eq.) and Cs₂CO₃ (32 mg, 0.098 mmol, 2.0 eq.) at 0° C. The reaction mixture was stirred at room temperature for 2 hrs. After completion, the reaction mixture was diluted with H₂O (10 mL) and extracted with DCM (10 mL×2). The combined organic layers were washed with brine (10 mL) and dried over Na₂SO₄, concentrated under reduced pressure, and the residue was purified by Prep-TLC to afford 5-(cyclopropylmethyl)-4-(4-(difluoromethoxy)phenyl)-7-methyl-2-(2-methyl-2H-indazol-5-yl)-2,7-dihydro-3H-pyrrolo[2,3-c]pyridazin-3-one (Example 272). ¹H NMR (400 MHz, DMSO-d₆) δ (ppm): 8.44 (s, 1H), 7.94-7.91 (m, 1H), 7.65 (d, J=9.6 Hz, 2H), 7.51-7.47 (m, 2H), 7.41 (dd, J=9.1, 2.0 Hz, 1H), 7.32 (t, J_{HF}=74 Hz, 1H), 7.24 (d, J=8.6 Hz, 2H), 4.20 (s, 3H), 3.54 (s, 3H), 1.95 (d, J=6.8 Hz, 2H), 0.49-0.44 (m, 1H), 0.28-0.23 (m, 2H), −0.14-−0.1 (m, 2H). LC-MS (ESI): m/z 476 [M+H]⁺.

### Example 273: Biochemical Assays

Mat2A protein was expressed by recombinant baculovirus in SF9 infected cells using the Bac to Bac system cloned into the pFASTBAC1 vector (Invitrogen, Carlsbad, Calif.). Recombinant MAT2A was isolated from the cell lysate of 150 g of infected cells using HP Ni sepharose column chromatography. Recombinant MAT2A homodimer was eluted with 250 and 500 mM imidazole, and fractions containing MAT2A were identified by sodium dodecyl sulfate polyacrylamide gel electrophoresis and pooled.

A. MAT2A Inhibition

For determination of the inhibitory potency of compounds against the MAT2A homodimer, protein was diluted to 4 μg/mL in assay buffer (50 mM Tris, pH 8.0, 50 mM KCl, 15 mM MgCl₂, 0.3 mM EDTA, 0.005% [w/v] bovine serum albumin [BSA]). Test compound was prepared in 100% dimethyl sulfoxide (DMSO) at 50× the desired final concentration. A 1 μL volume of compound dilution was added to 40 μL of enzyme dilution and the mixture was allowed to equilibrate for 60 minutes at 25° C. The enzymatic assay was initiated by the addition of 10 μL of substrate mix (500 μM ATP, pH 7.0, 400 μM L-methionine in 1× assay buffer), and the mixture was incubated for a further 60 minutes at 25° C. The reaction was halted and the liberated phosphate released by the enzyme in stoichiometric amounts by the production of S-adenosyl methionine (SAM) was measured using the PiColorLock Gold kit (Innova Biosciences, UK). Absolute product amounts were determined by comparison to a standard curve of potassium phosphate buffer, pH 8.0. The compounds were also tested in a secondary assay utilizing 1 μg/mL MAT2A and incubated for 5 hours at 25° C. before halting and processing as above.

Specific compounds disclosed herein were tested in the foregoing assay and they were determined to inhibit MAT2A with an IC₅₀. See, Table 1 below.

B. Cellular Assay of Target Engagement (SAM)

Measurement of MAT2A activity in cells was made by direct quantitation of the abundance of the product of its enzymatic activity, SAM. Cancer cells were treated with candidate MAT2A inhibitors for a suitable incubation period, and the cells were then lysed using a reagent which quenched any further enzyme activity. Soluble metabolites including SAM were collected and SAM itself was directly measured from the lysate using quantitative LC-MS/MS.

A typical assay was performed using an HCT116 human colon carcinoma cell line which was genetically engineered to delete the MTAP gene (commercially available from Horizon Discovery). This cell line was utilized because it was determined that loss of the MTAP gene predicts sensitivity to MAT2A inhibitors. Cells were plated in 96-well dishes at appropriate cell density. Following 24 hours, cells were then treated with the candidate MAT2A inhibitor. Prior to addition to cells, the compound was first serially diluted in 100% DMSO, typically as a 3-fold serial dilution starting at 500× top dose with 10 dose points including DMSO only control. Compound was then transferred to a working stock plate in cell culture media by adding 5 μL of compound in DMSO to 495 μL of cell culture media. This working stock was then added to cells via a further 5-fold dilution, by adding 25 μL of working stock to 100 μL of cells in culture media. Following compound addition, cells were incubated at 37° C./5% CO₂ for 72 hrs.

To quantitate SAM levels following compound treatment, cells were gently washed once in ammonium carbonate buffer (75 mM at pH 7.4), placed on dry ice, and lysed with metabolite extraction buffer (80% cold methanol and 20% water (v/v) with acetic acid at 1M final concentration with 200 ng/mL deuterated d3-SAM as internal control). Following centrifugation at 4° C. at 3,200 rpm for 30 minutes, the supernatant was collected and stored at −80° C. until analysis by Liquid Chromatography with tandem Mass Spectrometry (LC-MS/MS). LC-MS/MS analysis was performed using an API6500 Mass Spectrometer (Sciex, Framingham, Mass., USA) operating in positive ion spray mode and equipped with a Waters UPLC Acquity (Waters, Milford, Mass., USA) BEH Amide column. Multiple Reaction Monitoring data was acquired for SAM and the d3-SAM standard, using a mass transition pair at m/z 399.2→250.1 and 402.2→250.1, respectively. In a typical LC-MS/MS analysis, the initial flow rate was 0.5 ml/min of 25% mobile phase A (acetonitrile and water at 5:95 (v/v) with 1% formic acid and 10 mM ammonium acetate) and 75% mobile phase B (acetonitrile and water at 95:5 (v/v) with 1% formic acid and 10 mM ammonium acetate), 0.2-0.5 minutes with 75%-35% mobile phase B, 25%-65% mobile phase A, at 0.5 min 65% mobile phase A and 35% mobile phase B, 1.0-1.1 minutes with 35%-75% mobile phase B, 65%-25% mobile phase A, at 1.1 min 25% mobile phase A and 75% mobile phase B with a total run time of 1.5 minutes.

Specific compounds disclosed herein were tested in the foregoing assay and they were determined to inhibit SAM with an IC₅₀. See, Table 1 below.

C. Assay for Inhibition of Cellular Proliferation

Test compound impact on cancer cell growth was assessed by treating cancer cells with compound for 4 days and then measuring proliferation using an ATP-based cell proliferation readout (Cell Titer Glo, Promega Corporation).

In a typical assay an isogenic pair of HCT116 human colon carcinoma cell lines which vary only in MTAP deletion status (HCT116 MTAP+/+ and HCT116 MTAP−/−) were plated in 96-well dishes at appropriate cell density. Following 24 hours, cells were then treated with the candidate MAT2A inhibitor. Prior to addition to cells, the compound was first serially diluted in 100% DMSO, typically as a 3-fold serial dilution starting at 500× top dose with 10 dose points including DMSO only control. Compound was then transferred to a working stock plate in cell culture media by adding 5 μL of compound in DMSO to 495 μL of cell culture media. This working stock was then added to cells via a further 5-fold dilution, by adding 25 μL of working stock to 100 μL of cells in culture media. Following compound addition, cells were incubated at 37° C./5% CO₂ for 4 days.

To measure inhibition of cellular proliferation, cells were allowed to equilibrate to room temperature for 30 minutes, and were then treated with 125 μL of Cell Titer Glo reagent. The plate was then covered with aluminum foil and shaken for 15 minutes to ensure complete mixing and full cell lysis. Luminescent signal was then measured using a plate-based luminometer Veritas version 1.9.2 using ATP standard curve to confirm assay reproducibility from run to run. This luminescence measure was converted to a proliferation index by subtracting from each data point the ATP luminescence signal measured from a bank (no cells) well and dividing by the ATP luminescence signal measured in 0.2% DMSO control well adjusted for signal in blank well. Compound activity was then represented as a percentage change in proliferation relative to a within-plate DMSO control against log 10 of compound concentration in molar (M) units.

Specific compounds disclosed herein were tested in the foregoing assay and they were determined to inhibit cellular proliferation with an IC₅₀. See, Table 1 below.

TABLE 1

| | | | 4 Day Relative | 4 Day Relative |
|---|---|---|---|---|
| | Enzyme | Cell 72 h SAM | Growth | Growth |
| Example | Inhibition | Inhibition | Inhibition | Inhibition |
| No | Bin¹ | (MTAP −/−)² | (MTAP −/−)³ | (MTAP +/+)⁴ |
| 105 | A | A | A | C |
| 101 | A | A | A | C |
| 102 | A | A | A | C |
| 106 | A | A | A | C |
| 107 | A | A | A | B |
| 103 | A | A | A | B |
| 110 | A | A | A | C |
| 104 | A | A | A | C |
| 111 | A | A | A | C |
| 112 | A | A | A | C |
| 113 | A | A | A | C |
| 114 | A | A | A | C |
| 115 | A | A | A | C |
| 116 | A | A | B | C |
| 121 | E | NT | NT | NT |
| 122 | A | A | A | C |
| 123 | A | A | A | B |
| 124 | A | A | B | C |
| 125 | D | NT | NT | NT |
| 126 | E | NT | NT | NT |
| 127 | A | A | B | C |
| 118 | A | A | A | C |
| 128 | A | A | B | C |
| 129 | A | B | A | C |
| 130 | A | A | B | C |
| 131 | A | A | B | C |
| 132 | A | B | B | C |
| 133 | A | B | B | C |
| 134 | A | A | B | C |
| 108 | B | B | C | B |
| 135 | A | A | A | B |
| 136 | A | A | A | B |
| 137 | A | B | B | C |
| 109 | A | B | B | C |
| 138 | A | A | A | C |
| 119 | A | C | C | C |
| 139 | A | A | A | A |
| 140 | A | A | A | B |
| 141 | A | A | A | B |
| 142 | A | A | A | C |
| 143 | A | A | A | B |
| 144 | B | B | C | C |
| 145 | A | B | B | B |
| 146 | E | NT | NT | NT |
| 147 | A | A | A | B |
| 148 | A | A | A | A |
| 149 | A | B | B | C |
| 150 | A | A | A | C |
| 151 | A | A | A | C |
| 152 | A | A | A | C |
| 153 | B | B | C | C |
| 154 | A | B | B | B |
| 155 | A | B | B | C |
| 186 | A | A | A | B |
| 187 | A | B | B | C |
| 233 | A | A | A | B |
| 234 | A | A | A | A |
| 156 | A | A | A | A |
| 157 | A | A | A | B |
| 158 | A | B | B | C |
| 159 | A | A | B | C |
| 235 | A | A | A | C |
| 236 | A | A | A | C |
| 237 | A | A | A | B |
| 163 | E | C | B | C |
| 162 | A | A | A | B |
| 238 | A | A | A | C |
| 117 | A | A | A | C |
| 239 | A | A | A | A |
| 240 | A | A | A | B |
| 188 | A | A | A | B |
| 164 | E | NT | NT | NT |
| 189 | A | B | B | C |
| 165 | A | A | A | B |
| 241 | A | A | A | C |
| 242 | A | B | B | B |
| 243 | A | A | A | C |
| 190 | A | A | A | C |
| 246 | A | A | A | B |
| 244 | A | A | A | B |
| 247 | A | A | A | C |
| 248 | A | A | A | C |
| 120 | A | B | C | C |
| 245 | A | C | B | C |
| 249 | A | A | A | C |
| 250 | A | A | A | C |
| 191 | A | A | A | C |
| 251 | A | A | A | B |
| 192 | A | A | A | B |
| 193 | A | A | A | C |
| 194 | A | A | A | B |
| 208 | A | A | B | B |
| 252 | A | B | B | C |
| 253 | A | B | A | C |
| 254 | A | B | B | B |
| 255 | A | A | A | B |
| 256 | A | B | B | C |
| 166 | A | A | A | C |
| 258 | A | A | A | B |
| 160 | A | A | A | B |
| 259 | A | A | A | C |
| 197 | A | A | B | B |
| 177 | A | A | B | C |
| 196 | A | A | A | B |
| 260 | C | C | C | C |
| 257 | A | B | B | C |
| 209 | A | B | B | C |
| 261 | E | NT | NT | NT |
| 262 | E | NT | NT | NT |
| 167 | A | A | A | A |
| 263 | E | A | C | C |
| 210 | B | C | B | C |
| 179 | D | NT | NT | NT |
| 211 | A | A | A | C |
| 195 | A | A | A | C |
| 265 | D | NT | NT | NT |
| 180 | A | A | A | C |
| 212 | A | A | A | A |
| 213 | A | A | A | C |
| 214 | A | A | A | B |
| 267 | A | B | B | C |
| 181 | D | NT | NT | NT |
| 178 | B | C | C | C |
| 168 | A | A | A | C |
| 169 | A | A | A | B |
| 268 | A | A | A | C |
| 269 | A | A | C | C |
| 182 | D | NT | NT | NT |
| 266 | D | NT | NT | NT |
| 218 | A | A | A | B |
| 215 | A | A | A | B |
| 183 | D | NT | NT | NT |
| 184 | A | A | B | C |
| 185 | A | B | B | C |
| 170 | A | A | A | C |
| 171 | A | A | A | A |
| 198 | A | A | A | C |
| 264 | A | A | A | C |
| 203 | A | A | A | B |
| 216 | A | A | A | B |
| 199 | A | A | A | C |
| 200 | A | A | A | B |
| 204 | A | A | A | C |
| 205 | A | A | B | C |
| 172 | A | A | A | C |
| 219 | A | B | B | B |
| 217 | A | A | A | C |
| 201 | A | A | A | B |
| 173 | A | A | A | C |
| 174 | A | A | A | B |
| 206 | A | A | B | C |
| 270 | A | A | A | B |
| 207 | A | A | B | C |
| 202 | A | B | B | B |
| 220 | A | A | A | B |
| 175 | A | B | B | C |
| 221 | A | A | A | B |
| 176 | A | A | A | B |
| 223 | A | A | B | B |
| 225 | A | A | A | B |
| 161 | A | A | A | C |
| 226 | A | A | A | B |
| 227 | A | A | A | B |
| 228 | A | B | C | C |
| 229 | A | A | A | B |
| 230 | A | B | B | C |
| 222 | A | B | B | C |
| 224 | A | A | A | B |
| 231 | A | B | B | C |
| 232 | D | NT | NT | NT |
| 271 | A | A | A | B |
| 272 | A | NT | NT | NT |

* NT: Not Tested
¹A: <100 nM (>40% Max Inh.); B: 100 nM-1 μM (>40% Max Inh.); C: 1 μM-10 μM (>40% Max Inh.); D: >10 μM; E: <1 μM but <40% Max Inh.
²A: <100 nM (>40% Max Inh.); B: 100 nM-1 μM (>40% Max Inh.); C: >1 μM
³A: <100 nM (>10% Max Inh.); B: 100 nM-1 μM (>10% Max Inh.); C: >1 μM
⁴A: <100 nM (>10% Max Inh.); B: 100 nM-1 μM (>10% Max Inh.); C: >1 μM

The disclosure of each patent, patent application, and publication cited or described herein are hereby incorporated herein by reference, each in its entirety.

What is claimed is:

1. A compound of Formula I, or a pharmaceutically acceptable salt thereof: 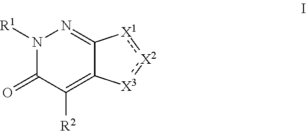
wherein:
X¹ is N, NR⁶, CR⁴, or CR⁴R^{4′};
X² is N, CR⁵, or CR⁵R⁵;
X³ is N, NR³, or CR³;
X¹ [symbol] X² and X² [symbol] X³ are, independently, a double or single bond, provided that (i) both X¹ [symbol] X² and X² [symbol] X³ are not double bonds and (i) both X¹ [symbol] X² and X² [symbol] X³ are single bonds when X² is C(O);
R¹ is C₆₋₁₄-aryl or 3- to 14-membered heteroaryl, wherein each is optionally substituted, as permitted, by one or more of R^{A};
R² is —C₀₋₆alkyl-C₆₋₁₄aryl or —C₀₋₆alkyl-(3- to 14-membered heteroaryl), wherein each, as permitted, is optionally substituted by one or more of R^{A};
R³ is H, D, CN, halo, OH, C₁₋₆alkyl, C₁₋₆alkylOH, deuterated C₁₋₆alkyl, C₂₋₆alkenyl, C₂₋₆haloalkenyl, C₂₋₆alkynyl, C₁₋₆alkoxy, deuterated C₁₋₆alkoxy, C₁₋₆haloalkoxy, C₁₋₆haloalkyl, —C₀₋₆alkyl-C₃₋₁₂carbocyclyl, —C₀₋₆alkyl-C₆₋₁₄aryl, —C₀₋₆alkyl-O—C₆₋₁₄aryl, —C₀₋₆alkyl-(3- to 14-membered heterocyclyl), —C₀₋₆alkyl-O-(3- to 14-membered heterocyclyl), —C₀₋₆alkyl-(3- to 14-membered heteroaryl), —C₀₋₆alkyl-NR^{A}(CH₂)₀₋₆C(O)R^{A} NR^{B}R^{C}, C(O)NR^{B}R^{C}, NR^{B}C(NR^{C})NR^{B}R^{C}, NR^{B}C(NR^{C})(═NR^{B}), —S(O)₀₋₂(C₁₋₆alkyl), —S(O)₀₋₂(C₁₋₆alkyl)₂, —S(O)₀₋₂NH₂, —S(O)₀₋₂NHC₁₋₆alkyl, —S(O)₀₋₂(C₆₋₁₄aryl), —S(O)₀₋₂(heteroaryl), —S(O)₀₋₂(heterocyclyl), —C(O)(C₁₋₆alkyl), —C(O)(C₃₋₁₄carbocyclyl), —C(O)NR^{A}R^{B}, —C(O)(C₆₋₁₄aryl), —C(O)(heteroaryl), —C(O)(heterocyclyl), or SR^{B} wherein each of the forgoing, as permitted, is optionally substituted by one or more of R^{A};
R⁴, R^{4′}, R⁵, and R^{5′} are, independently, H, D, CN, OH, C₁₋₆alkyl, halo, deuterated C₁₋₆alkyl, C₁₋₆alkoxy, deuterated C₁₋₆alkoxy, C₁₋₆alkylOH, C₁₋₆haloalkyl, C₁₋₆haloalkoxy, C₂₋₆alkenyl, C₂₋₆haloalkenyl, C₂₋₆alkynyl, —S(O)₀₋₂NH₂, —S(O)₀₋₂NHC₁₋₆alkyl, —S(O)₀₋₂(C₁₋₆alkyl)₂, —S(O)₀₋₂C₁₋₆alkyl, —S(O)₀₋₂(C₆₋₁₄aryl), —S(O)₀₋₂(heteroaryl), —S(O)₀₋₂(heterocyclyl), —C₀₋₆alkyl-(C₃₋₁₄carbocyclyl), —C₀₋₆alkyl-C₆₋₁₄aryl, —C₀₋₆alkyl-O—C₆₋₁₄aryl, C₀₋₆alkyl-(3- to 14-membered heterocyclyl), —C₀₋₆alkyl-O-(3- to 14-membered heterocyclyl), C₀₋₆alkyl-(3- to 14-membered heteroaryl), —C₁₋₆alkyl-NR^{A}(CH₂)₀₋₆C(O)R^{A}, NR^{B}R^{C}, C(O)NR^{B}R^{C}, NR^{B}C(NR^{C})NR^{B}R^{C}, NR^{B}C(NR^{C})(═NR^{B}), —C(O)NR^{A}R^{B}, —C(O)(C₁₋₆alkyl), —C(O)(C₃₋₁₄carbocyclyl), —C(O)(heteroaryl), —C(O)(heterocyclyl), —C(O)(C₆₋₁₄aryl), or SR^{B} wherein each of the forgoing, as permitted, is optionally substituted by one or more R^{A};
or R⁴ and R^{4′} or R⁵ and R^{5′} are combined to form oxo; and
R⁶ is H or C₁₋₆alkyl;
R^{A} is D, CN, NH₂, halo, C₁₋₆alkyl, deuterated C₁₋₆alkyl, C₁₋₆haloalkyl, C₁₋₆alkoxy, C₁₋₆haloalkoxy, deuterated C₁₋₆alkoxy, OH, —C₁₋₆alkylOH, C₂₋₆alkenyl, C₂₋₆haloalkenyl, C₂₋₆alkynyl, —S(O)₀₋₂(C₁₋₆alkyl), —S(O)₀₋₂(C₆₋₁₄aryl), —S(O)₀₋₂NH₂, —S(O)₀₋₂NHC₁₋₆alkyl, —S(O)₀₋₂(C₁₋₆alkyl)₂, —S(O)₀₋₂(heteroaryl), —S(O)₀₋₂(heterocyclyl), —C(O)(C₁₋₆alkyl), —C(O)NH₂, —C(O)NHC₁₋₆alkyl, —C(O)NH(C₁₋₆alkyl)₂, —C(O)(C₃₋₁₄carbocyclyl), —C(O)(heteroaryl), —C(O)(heterocyclyl), —C(O)(C₆₋₁₄aryl), —C₀₋₆alkyl-C₃₋₁₄carbocyclyl, —C₀₋₆alkyl-C₆₋₁₄aryl, —C₀₋₆alkyl-O—C₆₋₁₄aryl, —C₀₋₆alkyl-(3- to 14-membered heterocyclyl), —C₀₋₆alkyl-(3- to 14-membered heteroaryl), or —C₀₋₆alkyl-O-(3- to 14-membered heterocyclyl),
wherein R^{A}, as permitted, is optionally substituted by D, CN, halo, C₁₋₆alkyl, deuterated C₁₋₆alkyl, C₁₋₆haloalkyl, —C₀₋₆alkyl-C₃₋₁₄carbocyclyl, C₁₋₆alkoxy, C₁₋₆haloalkoxy, deuterated C₁₋₆alkoxy, OH, —C₁₋₆alkylOH, C₂₋₆alkenyl, C₂₋₆haloalkenyl, C₂₋₆alkynyl, —S(O)₀₋₂(C₁₋₆alkyl), —S(O)₀₋₂(C₆₋₁₄aryl), —S(O)₀₋₂(heteroaryl), —S(O)₀₋₂(heterocyclyl), —S(O)₀₋₂NH₂, —S(O)₀₋₂NHC₁₋₆alkyl, —S(O)₀₋₂(C₁₋₆alkyl)₂, —C(O)(C₁₋₆alkyl), —C(O)NH₂, —C(O)NHC₁₋₆alkyl, —C(O)NH(C₁₋₆alkyl)₂, —C(O)(C₃₋₁₄carbocyclyl), —C(O)(C₆₋₁₄aryl), —C(O)(heteroaryl), —C(O)(heterocyclyl), —C₀₋₆alkyl-O—C₆₋₁₄aryl, —C₀₋₆alkyl-C₆₋₁₄aryl, —C₀₋₆alkyl-(3- to 14-membered heterocyclyl), —C₀₋₆alkyl-(3- to 14-membered heteroaryl), —C₀₋₆alkyl-O-(3- to 14-membered heterocyclyl), —C₀₋₆alkyl-NH₂, —C₀₋₆alkyl-NH(alkyl), or —C₀₋₆alkyl-N(alkyl)₂;
R^{B} and R^{C} are, independently, H, D, CN, NH₂, halo, OH, C₁₋₆alkyl, deuterated C₁₋₆alkyl, C₁₋₆haloalkyl, —C₀₋₆alkyl-C₃₋₁₄carbocyclyl, C₁₋₆alkoxy, C₁₋₆haloalkoxy, deuterated C₁₋₆alkoxy, —C₁₋₆alkylOH, C₂₋₆alkenyl, C₂₋₆haloalkenyl, C₂₋₆alkynyl, —S(O)₀₋₂(C₁₋₆alkyl), —S(O)₀₋₂NH₂, —S(O)₀₋₂NHC₁₋₆alkyl, —S(O)₀₋₂(C₁₋₆alkyl)₂, —S(O)₀₋₂(C₆₋₁₄aryl), —S(O)₀₋₂(heteroaryl), —S(O)₀₋₂(heterocyclyl), —C(O)(C₁₋₆alkyl), —C(O)NH₂, —C(O)NHC₁₋₆alkyl, —C(O)NH(C₁₋₆alkyl)₂, —C(O)(C₃₋₁₄carbocyclyl), —C(O)(heteroaryl), —C(O)(heterocyclyl), —C(O)(C₆₋₁₄aryl), —C₀₋₆alkyl-C₆₋₁₄aryl, —C₀₋₆alkyl-O—C₆₋₁₄aryl, —C₀₋₆alkyl-(3- to 14-membered heterocyclyl), —C₀₋₆alkyl-(3- to 14-membered heteroaryl), —C₀₋₆alkyl-O-(3- to 14-membered heterocyclyl), —C₀₋₆alkyl-NH₂, —C₀₋₆alkyl-NH(alkyl), or —C₀₋₆alkyl-N(alkyl)₂;
wherein each of the forgoing, as permitted, is optionally substituted by D, CN, halo, OH, C₁₋₆alkyl, deuterated C₁₋₆alkyl, C₁₋₆haloalkyl, —C₀₋₆alkyl-C₃₋₁₄carbocyclyl, C₁₋₆alkoxy, C₁₋₆haloalkoxy, deuterated C₁₋₆alkoxy, —C₁₋₆alkylOH, C₂₋₆alkenyl, C₂₋₆haloalkenyl, C₂₋₆alkynyl, —S(O)₀₋₂(C₁₋₆alkyl), —S(O)₀₋₂(C₆₋₁₄aryl), —S(O)₀₋₂(heteroaryl), —S(O)₀₋₂(heterocyclyl), —S(O)₀₋₂NH₂, —S(O)₀₋₂NHC₁₋₆alkyl, —S(O)₀₋₂(C₁₋₆alkyl)₂, —C(O)(C₁₋₆alkyl), —C(O)NH₂, —C(O)NHC₁₋₆alkyl, —C(O)NH(C₁₋₆alkyl)₂, —C(O)(C₃₋₁₄carbocyclyl), —C(O)(C₆₋₁₄aryl), —C(O)(heteroaryl), —C(O)(heterocyclyl), —C₀₋₆alkyl-O—C₆₋₁₄aryl, —C₀₋₆alkyl-C₆₋₁₄aryl, —C₀₋₆alkyl-(3- to 14-membered heterocyclyl), —C₀₋₆alkyl-3- to 14-membered heteroaryl, or —C₀₋₆alkyl-O-(3- to 14-membered heterocyclyl), —C₀₋₆alkyl-NH₂, —C₀₋₆alkyl-NH(alkyl), or —C₀₋₆alkyl-N(alkyl)₂;
wherein each heteroaryl or heterocyclyl, independently, comprises 1 to 4 heteroatoms that are, independently, N, O, or S.

2. The compound of claim 1, wherein R¹ is optionally substituted C₆₋₁₄-aryl.

3. The compound of claim 1, wherein R¹ is optionally substituted 3- to 14-membered heteroaryl, optionally comprising at least one N, or one N and one S, or two N, or one N and one O.

4. The compound of claim 1, wherein R¹ is substituted with one or more of C₁₋₆alkyl, deuterated C₁₋₆alkyl, C₁₋₆haloalkyl, NH₂, C₁₋₆alkoxy, or —C₀₋₆alkyl-C₃₋₈carbocylyl.

5. The compound of claim 1, wherein R² is —C₀₋₆alkyl-optionally substituted C₆₋₁₄-aryl.

6. The compound of claim 1, wherein R² is —C₀₋₆alkyl-(optionally substituted 3- to 14-membered heteroaryl), optionally comprising at least one N, or one N and one S, or two N, or one N and one O.

7. The compound of claim 1, wherein R² is substituted with one or more of halo, CN, C₁₋₆alkyl, deuterated C₁₋₆alkyl, C₁₋₆haloalkyl, C₁₋₆alkylOH, C₁₋₆alkoxy, C₁₋₆haloalkoxy, deuterated C₁₋₆alkoxy, C₂₋₆haloalkenyl, —C₀₋₆alkyl-C₃₋₁₀carbocyclyl, C(O)NH₂, or C(O)NHC₁₋₆alkyl.

8. The compound of claim 1, wherein (i) X¹ [symbol] X² and X² [symbol] X³ are single bonds; or (ii) X¹ [symbol] X² is a single bond and X² [symbol] X³ is a double bond; or (iii) X¹ [symbol] X² is a double bond and X² [symbol] X³ is a double bond.

9. The compound of claim 1, wherein X¹ is N or NR⁶.

10. The compound of claim 1, wherein R⁶ is H.

11. The compound of claim 1, wherein R⁶ is C₁₋₆alkyl

12. The compound of claim 1, wherein X¹ is CR⁴ or CR⁴R^{4′}.

13. The compound of claim 1, wherein R⁴ is H, CN, C₁₋₆alkyl, halo, C₁₋₆alkylOH, C₁₋₆haloalkyl, C₂₋₆alkynyl, C(O)OH, C(O)C₁₋₆alkyl, C(O)NH₂, C(O)NHC₁₋₆alkyl, C(O)N(C₁₋₆alkyl)₂, S(O)₀₋₂NH₂, S(O)₀₋₂NHC₁₋₆alkyl, S(O)₀₋₂N(C₁₋₆alkyl)₂, or S(O)₀₋₂C₁₋₆alkyl.

14. The compound of claim 1, wherein X² and/or X³ is N.

15. The compound of claim 1, wherein X² is CR⁵ or CR⁵R^{5′}.

16. The compound of claim 1, wherein R⁵ is H, halo, or C₁₋₆alkyl.

17. The compound of claim 15, wherein R⁵ and R^{5′} are combined to form oxo.

18. The compound of claim 1, wherein X³ is NR³ or CR³.

19. The compound of claim 1, wherein R³ is H, C₁₋₆alkyl, C₁₋₆haloalkyl, or —C₀₋₆alkyl-C₃₋₈carbocyclyl.

20. The compound of claim 1, having the structure of Formula II, III, IV, V, VI, VII, VIII, IX, or X: 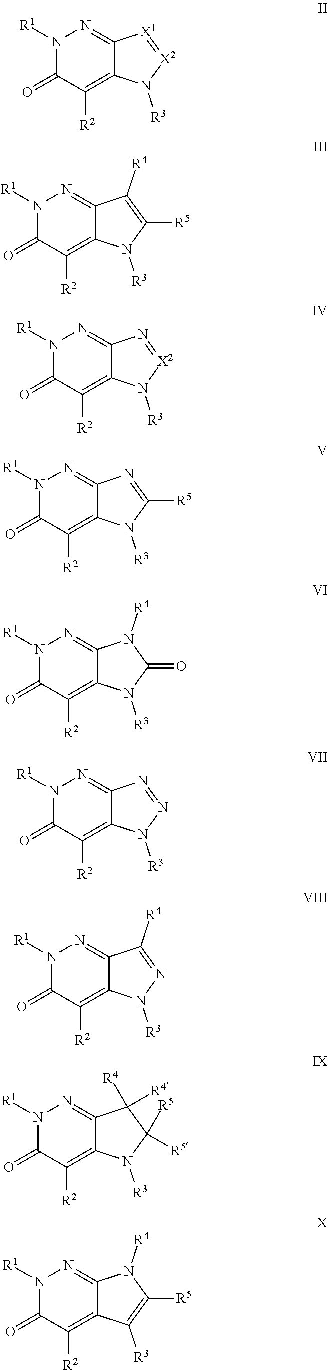
or a pharmaceutically acceptable salt thereof.

21. The compound of claim 1, wherein:
R¹ is substituted indazolyl, substituted benzimidazolyl or benzothiazole;
R² is substituted phenyl or substituted pyridyl;
X² is CR⁵ or CR⁵R^{5′}; and
X³ is N or NR³.

22. The compound of claim 1, having the structure of Formula XIa, XIb, XIc, XIIa, XIIb, XIIc, XIIIa, XIIIb, or XIIIc: 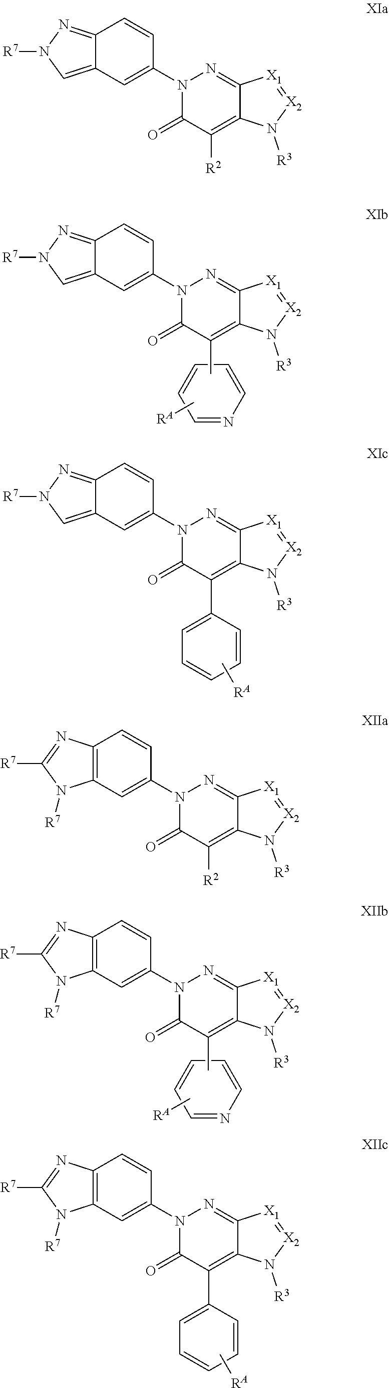
wherein R⁷ is H, C₁₋₆alkyl, deuterated C₁₋₆alkyl, —C₁₋₆alkyl-C₃₋₈carbocyclyl, C₁₋₆alkoxy, or deuterated C₁₋₆alkoxy;
or a pharmaceutically acceptable salt thereof.

23. The compound of claim 1, or a pharmaceutically acceptable salt thereof, that is:
Example 101: 5-(cyclopropylmethyl)-4-(4-(difluoromethoxy)phenyl)-2-(2-methyl-2H-indazol-5-yl)-2,5-dihydro-3H-pyrrolo[3,2-c]pyridazin-3-one;
Example 102: 5-(cyclopropylmethyl)-4-(4-(difluoromethoxy)phenyl)-2-(2-methyl-2H-indazol-5-yl)-3-oxo-3,5-dihydro-2H-pyrrolo[3,2-c]pyridazine-7-carbonitrile;
Example 103: 5-(cyclopropylmethyl)-2-(2-methyl-2H-indazol-5-yl)-4-(6-methylpyridin-3-yl)-2,5-dihydro-3H-pyrrolo[3,2-c]pyridazin-3-one;
Example 104: 5-(cyclopropylmethyl)-2-(2-methyl-2H-indazol-5-yl)-4-(6-methylpyridin-3-yl)-3-oxo-3,5-dihydro-2H-pyrrolo[3,2-c]pyridazine-7-carbonitrile;
Example 105: 4-(4-(difluoromethoxy)phenyl)-2-(2-methyl-2H-indazol-5-yl)-5-(3,3,3-trifluoropropyl)-2,5-dihydro-3H-pyrrolo[3,2-c]pyridazin-3-one;
Example 106: 5-(cyclopropylmethyl)-4-(6-cyclopropylpyridin-3-yl)-2-(2-methyl-2H-indazol-5-yl)-2,5-dihydro-3H-pyrrolo[3,2-c]pyridazin-3-one;
Example 107: 5-(cyclopropylmethyl)-4-(6-cyclopropylpyridin-3-yl)-2-(2-methyl-2H-indazol-5-yl)-3-oxo-3,5-dihydro-2H-pyrrolo[3,2-c]pyridazine-7-carbonitrile;
Example 108: 2-(2-methyl-2H-indazol-5-yl)-4-(6-methylpyridin-3-yl)-3-oxo-5-(3,3,3-trifluoropropyl)-3,5-dihydro-2H-pyrrolo[3,2-c]pyridazine-7-carbonitrile;
Example 109: 4-(6-cyclopropylpyridin-3-yl)-2-(2-methyl-2H-indazol-5-yl)-3-oxo-5-(3,3,3-trifluoropropyl)-3,5-dihydro-2H-pyrrolo[3,2-c]pyridazine-7-carbonitrile;
Example 110: 5-(cyclopropylmethyl)-4-(6-methoxypyridin-3-yl)-2-(2-methyl-2H-indazol-5-yl)-2,5-dihydro-3H-pyrrolo[3,2-c]pyridazin-3-one;
Example 111: 5-(cyclopropylmethyl)-4-(6-methoxypyridin-3-yl)-2-(2-methyl-2H-indazol-5-yl)-3-oxo-3,5-dihydro-2H-pyrrolo[3,2-c]pyridazine-7-carbonitrile;
Example 112: 4-(4-chlorophenyl)-5-(cyclopropylmethyl)-2-(2-methyl-2H-indazol-5-yl)-2,5-dihydro-3H-pyrrolo[3,2-c]pyridazin-3-one;
Example 113: 5-(cyclopropylmethyl)-4-(6-(difluoromethoxy)pyridin-3-yl)-2-(2-methyl-2H-indazol-5-yl)-2,5-dihydro-3H-pyrrolo[3,2-c]pyridazin-3-one;
Example 114: 5-(cyclopropylmethyl)-4-(6-(difluoromethoxy)pyridin-3-yl)-2-(2-methyl-2H-indazol-5-yl)-3-oxo-3,5-dihydro-2H-pyrrolo[3,2-c]pyridazine-7-carbonitrile;
Example 115: 4-(4-(difluoromethoxy)phenyl)-5-isobutyl-2-(2-methyl-2H-indazol-5-yl)-2,5-dihydro-3H-pyrrolo[3,2-c]pyridazin-3-one;
Example 116: 4-(4-(difluoromethoxy)phenyl)-5-isobutyl-2-(2-methyl-2H-indazol-5-yl)-3-oxo-3,5-dihydro-2H-pyrrolo[3,2-c]pyridazine-7-carbonitrile;
Example 117: 5-(cyclopropylmethyl)-4-(6-cyclopropylpyridin-3-yl)-7-iodo-2-(2-methyl-2H-indazol-5-yl)-2,5-dihydro-3H-pyrrolo[3,2-c]pyridazin-3-one;
Example 118: 5-(cyclopropylmethyl)-2-(2-methyl-2H-indazol-5-yl)-4-(6-(trifluoromethyl)pyridin-3-yl)-2,5-dihydro-3H-pyrrolo[3,2-c]pyridazin-3-one;
Example 119: 5-(cyclopropylmethyl)-2-(2-methyl-2H-indazol-5-yl)-3-oxo-4-(6-(trifluoromethyl)pyridin-3-yl)-3,5-dihydro-2H-pyrrolo[3,2-c]pyridazine-7-carbonitrile;
Example 120: 4-(6-(1-cyanocyclopropyl)pyridin-3-yl)-5-(cyclopropylmethyl)-2-(2-methyl-2H-indazol-5-yl)-3-oxo-3,5-dihydro-2H-pyrrolo[3,2-c]pyridazine-7-carbonitrile;
Example 121: 4-(6-cyclopropylpyridin-3-yl)-5-isobutyl-2-(2-methyl-2H-indazol-5-yl)-3-oxo-3,5-dihydro-2H-pyrrolo[3,2-c]pyridazine-7-carbonitrile;
Example 122: 5-(cyclopropylmethyl)-4-(6-cyclopropylpyridin-3-yl)-2-(4-(methoxy-d3)phenyl)-2,5-dihydro-3H-pyrrolo[3,2-c]pyridazin-3-one;
Example 123: 5-(cyclopropylmethyl)-4-(6-(difluoromethyl)pyridin-3-yl)-2-(2-methyl-2H-indazol-5-yl)-2,5-dihydro-3H-pyrrolo[3,2-c]pyridazin-3-one;
Example 124: 5-(cyclopropylmethyl)-4-(6-cyclopropylpyridin-3-yl)-2-(4-(methoxy-d3)phenyl)-3-oxo-3,5-dihydro-2H-pyrrolo[3,2-c]pyridazine-7-carbonitrile;
Example 125: 5-(cyclopropylmethyl)-2-(2-methyl-2H-indazol-5-yl)-4-(5-(trifluoromethyl)pyridin-3-yl)-2,5-dihydro-3H-pyrrolo[3,2-c]pyridazin-3-one;
Example 126: 4-(5-chloropyridin-3-yl)-5-(cyclopropylmethyl)-2-(2-methyl-2H-indazol-5-yl)-2,5-dihydro-3H-pyrrolo[3,2-c]pyridazin-3-one;
Example 127: 4-(6-cyclopropylpyridin-3-yl)-2-(2-methyl-2H-indazol-5-yl)-3-oxo-5-propyl-3,5-dihydro-2H-pyrrolo[3,2-c]pyridazine-7-carbonitrile;
Example 128: 5-(cyclopropylmethyl)-4-(6-isopropylpyridin-3-yl)-2-(2-methyl-2H-indazol-5-yl)-3-oxo-3,5-dihydro-2H-pyrrolo[3,2-c]pyridazine-7-carbonitrile;
Example 129: 5-(cyclopropylmethyl)-4-(6-isopropylpyridin-3-yl)-2-(2-methyl-2H-indazol-5-yl)-2,5-dihydro-3H-pyrrolo[3,2-c]pyridazin-3-one;
Example 130: 4-(3-cyano-1H-indol-5-yl)-5-(cyclopropylmethyl)-2-(2-methyl-2H-indazol-5-yl)-3-oxo-3,5-dihydro-2H-pyrrolo[3,2-c]pyridazine-7-carbonitrile;
Example 131: 5-(cyclopropylmethyl)-2-(4-(methoxy-d3)phenyl)-4-(6-methylpyridin-3-yl)-3-oxo-3,5-dihydro-2H-pyrrolo[3,2-c]pyridazine-7-carbonitrile;
Example 132: 5-(cyclopropylmethyl)-4-(6-(difluoromethyl)pyridin-3-yl)-2-(4-(methoxy-d3)phenyl)-2,5-dihydro-3H-pyrrolo[3,2-c]pyridazin-3-one;
Example 133: 4-(6-cyclopropylpyridin-3-yl)-2-(2-methyl-2H-indazol-5-yl)-3-oxo-5-(2,2,2-trifluoroethyl)-3,5-dihydro-2H-pyrrolo[3,2-c]pyridazine-7-carbonitrile;
Example 134: 5-(cyclopropylmethyl)-2-(4-(methoxy-d3)phenyl)-4-(6-methylpyridin-3-yl)-2,5-dihydro-3H-pyrrolo[3,2-c]pyridazin-3-one;
Example 135: 5-(cyclopropylmethyl)-4-(6-ethylpyridin-3-yl)-2-(2-methyl-2H-indazol-5-yl)-2,5-dihydro-3H-pyrrolo[3,2-c]pyridazin-3-one;
Example 136: 5-(cyclopropylmethyl)-4-(6-ethylpyridin-3-yl)-2-(2-methyl-2H-indazol-5-yl)-3-oxo-3,5-dihydro-2H-pyrrolo[3,2-c]pyridazine-7-carbonitrile;
Example 137: 5-(cyclopropylmethyl)-4-(6-(difluoromethyl)pyridin-3-yl)-2-(4-(methoxy-d3)phenyl)-3-oxo-3,5-dihydro-2H-pyrrolo[3,2-c]pyridazine-7-carbonitrile;
Example 138: 5-(cyclopropylmethyl)-4-(4-cyclopropylphenyl)-2-(2-methyl-2H-indazol-5-yl)-3-oxo-3,5-dihydro-2H-pyrrolo[3,2-c]pyridazine-7-carbonitrile;
Example 139: 4-(4-chlorophenyl)-5-(cyclopropylmethyl)-2-(2-methyl-2H-indazol-5-yl)-3-oxo-3,5-dihydro-2H-pyrrolo[3,2-c]pyridazine-7-carbonitrile;
Example 140: 5-(cyclopropylmethyl)-4-(6-(difluoromethyl)pyridin-3-yl)-2-(1,2-dimethyl-1H-benzo[d]imidazol-6-yl)-2,5-dihydro-3H-pyrrolo[3,2-c]pyridazin-3-one;
Example 141: 5-(cyclopropylmethyl)-2-(1,2-dimethyl-1H-benzo[d]imidazol-6-yl)-4-(6-methylpyridin-3-yl)-2,5-dihydro-3H-pyrrolo[3,2-c]pyridazin-3-one;
Example 142: 5-(cyclopropylmethyl)-2-(1,2-dimethyl-1H-benzo[d]imidazol-6-yl)-4-(6-ethylpyridin-3-yl)-2,5-dihydro-3H-pyrrolo[3,2-c]pyridazin-3-one;
Example 143: 5-(cyclopropylmethyl)-4-(6-(difluoromethyl)pyridin-3-yl)-2-(2-methyl-2H-indazol-5-yl)-3-oxo-3,5-dihydro-2H-pyrrolo[3,2-c]pyridazine-7-carbonitrile;
Example 144: 5-(5-(cyclopropylmethyl)-2-(2-methyl-2H-indazol-5-yl)-3-oxo-3,5-dihydro-2H-pyrrolo[3,2-c]pyridazin-4-yl)-N-methylpicolinamide;
Example 145: 2-(2-methyl-2H-indazol-5-yl)-4-(6-methylpyridin-3-yl)-3-oxo-5-propyl-3,5-dihydro-2H-pyrrolo[3,2-c]pyridazine-7-carbonitrile;
Example 146: 4-(6-cyclopropylpyridin-3-yl)-5-isopropyl-2-(2-methyl-2H-indazol-5-yl)-3-oxo-3,5-dihydro-2H-pyrrolo[3,2-c]pyridazine-7-carbonitrile;
Example 147: 5-(cyclopropylmethyl)-4-(6-(difluoromethyl)pyridin-3-yl)-2-(1,2-dimethyl-1H-benzo[d]imidazol-6-yl)-3-oxo-3,5-dihydro-2H-pyrrolo[3,2-c]pyridazine-7-carbonitrile;
Example 148: 5-(cyclopropylmethyl)-4-(6-cyclopropylpyridin-3-yl)-2-(1,2-dimethyl-1H-benzo[d]imidazol-6-yl)-3-oxo-3,5-dihydro-2H-pyrrolo[3,2-c]pyridazine-7-carbonitrile;
Example 149: 4-(4-cyanophenyl)-5-(cyclopropylmethyl)-2-(2-methyl-2H-indazol-5-yl)-3-oxo-3,5-dihydro-2H-pyrrolo[3,2-c]pyridazine-7-carbonitrile;
Example 150: 5-(cyclopropylmethyl)-4-(4-cyclopropylphenyl)-2-(2-methyl-2H-indazol-5-yl)-2,5-dihydro-3H-pyrrolo[3,2-c]pyridazin-3-one;
Example 151: 5-(cyclopropylmethyl)-2-(1,2-dimethyl-1H-benzo[d]imidazol-6-yl)-4-(6-methylpyridin-3-yl)-3-oxo-3,5-dihydro-2H-pyrrolo[3,2-c]pyridazine-7-carbonitrile;
Example 152: 5-(cyclopropylmethyl)-2-(1,2-dimethyl-1H-benzo[d]imidazol-6-yl)-4-(6-ethylpyridin-3-yl)-3-oxo-3,5-dihydro-2H-pyrrolo[3,2-c]pyridazine-7-carbonitrile;
Example 153: 5-(5-(cyclopropylmethyl)-2-(2-methyl-2H-indazol-5-yl)-3-oxo-3,5-dihydro-2H-pyrrolo[3,2-c]pyridazin-4-yl)picolinonitrile;
Example 154: 4-(5-(cyclopropylmethyl)-2-(2-methyl-2H-indazol-5-yl)-3-oxo-3,5-dihydro-2H-pyrrolo[3,2-c]pyridazin-4-yl)benzonitrile;
Example 155: 5-(5-(cyclopropylmethyl)-2-(2-methyl-2H-indazol-5-yl)-3-oxo-3,5-dihydro-2H-pyrrolo[3,2-c]pyridazin-4-yl)picolinamide;
Example 156: 5-(cyclopropylmethyl)-4-(6-cyclopropylpyridin-3-yl)-2-(1,2-dimethyl-1H-benzo[d]imidazol-6-yl)-2,5-dihydro-3H-pyrrolo[3,2-c]pyridazin-3-one;
Example 157: 5-(cyclopropylmethyl)-4-(6-(hydroxymethyl)pyridin-3-yl)-2-(2-methyl-2H-indazol-5-yl)-2,5-dihydro-3H-pyrrolo[3,2-c]pyridazin-3-one;
Example 158: 5-(cyclopropylmethyl)-4-(6-(hydroxymethyl)pyridin-3-yl)-2-(2-methyl-2H-indazol-5-yl)-3-oxo-3,5-dihydro-2H-pyrrolo[3,2-c]pyridazine-7-carbonitrile;
Example 159: 5-(cyclobutylmethyl)-4-(6-cyclopropylpyridin-3-yl)-2-(2-methyl-2H-indazol-5-yl)-3-oxo-3,5-dihydro-2H-pyrrolo[3,2-c]pyridazine-7-carbonitrile;
Example 160: 5-(cyclopropylmethyl)-2-(2-(cyclopropylmethyl)-2H-indazol-5-yl)-4-(6-cyclopropylpyridin-3-yl)-3-oxo-3,5-dihydro-2H-pyrrolo[3,2-c]pyridazine-7-carbonitrile;
Example 161: 5-(cyclopropylmethyl)-4-(4-(methoxy-d3)phenyl)-2-(2-methyl-2H-indazol-5-yl)-3-oxo-3,5-dihydro-2H-pyrrolo[3,2-c]pyridazine-7-carbonitrile;
Example 162: 5-(cyclopropylmethyl)-4-(6-cyclopropylpyridin-3-yl)-N-methyl-2-(2-methyl-2H-indazol-5-yl)-3-oxo-2H,3H,5H-pyrrolopyridazine-7-carboxamide;
Example 163: 5-(cyclopropylmethyl)-4-(6-cyclopropylpyridin-3-yl)-2-(2-methyl-2H-indazol-5-yl)-3-oxo-3,5-dihydro-2H-pyrrolo[3,2-c]pyridazine-7-carboxylic acid;
Example 164: 5-(cyclopropylmethyl)-4-(6-cyclopropylpyridin-3-yl)-N,N-dimethyl-2-(2-methyl-2H-indazol-5-yl)-3-oxo-3,5-dihydro-2H-pyrrolo[3,2-c]pyridazine-7-carboxamide;
Example 165: 5-(cyclopropylmethyl)-4-(6-cyclopropylpyridin-3-yl)-2-(2-methyl-2H-indazol-5-yl)-3-oxo-3,5-dihydro-2H-pyrrolo[3,2-c]pyridazine-7-carboxamide;
Example 166: 5-(cyclopropylmethyl)-4-(4-cyclopropylphenyl)-2-(2-methyl-2H-indazol-5-yl)-3-oxo-3,5-dihydro-2H-pyrrolo[3,2-c]pyridazine-7-carboxamide;
Example 167: 5-(cyclopropylmethyl)-4-(4-cyclopropylphenyl)-N-methyl-2-(2-methyl-2H-indazol-5-yl)-3-oxo-3,5-dihydro-2H-pyrrolo[3,2-c]pyridazine-7-carboxamide;
Example 168: 5-(cyclopropylmethyl)-4-(4-(difluoromethoxy)phenyl)-2-(2-methyl-2H-indazol-5-yl)-3-oxo-3,5-dihydro-2H-pyrrolo[3,2-c]pyridazine-7-carboxamide;
Example 169: 5-(cyclopropylmethyl)-4-(4-(difluoromethoxy)phenyl)-N-methyl-2-(2-methyl-2H-indazol-5-yl)-3-oxo-3,5-dihydro-2H-pyrrolo[3,2-c]pyridazine-7-carboxamide;
Example 170: 4-(4-chlorophenyl)-5-(cyclopropylmethyl)-2-(2-methyl-2H-indazol-5-yl)-3-oxo-3,5-dihydro-2H-pyrrolo[3,2-c]pyridazine-7-carboxamide;
Example 171: 4-(4-chlorophenyl)-5-(cyclopropylmethyl)-N-methyl-2-(2-methyl-2H-indazol-5-yl)-3-oxo-3,5-dihydro-2H-pyrrolo[3,2-c]pyridazine-7-carboxamide;
Example 172: 5-(cyclopropylmethyl)-4-(6-cyclopropylpyridin-3-yl)-N-methyl-2-(2-(methyl-d3)-2H-indazol-5-yl)-3-oxo-3,5-dihydro-2H-pyrrolo[3,2-c]pyridazine-7-carboxamide;
Example 173: 5-(cyclopropylmethyl)-4-(4-(methoxy-d3)phenyl)-2-(2-methyl-2H-indazol-5-yl)-3-oxo-3,5-dihydro-2H-pyrrolo[3,2-c]pyridazine-7-carboxamide;
Example 174: 5-(cyclopropylmethyl)-4-(4-(methoxy-d3)phenyl)-N-methyl-2-(2-methyl-2H-indazol-5-yl)-3-oxo-3,5-dihydro-2H-pyrrolo[3,2-c]pyridazine-7-carboxamide;
Example 175: 5-(cyclopropylmethyl)-2-(2-methyl-2H-indazol-5-yl)-4-(6-methylpyridin-3-yl)-3-oxo-3,5-dihydro-2H-pyrrolo[3,2-c]pyridazine-7-carboxamide;
Example 176: 5-(cyclopropylmethyl)-4-(4-methoxyphenyl)-N-methyl-2-(2-methyl-2H-indazol-5-yl)-3-oxo-3,5-dihydro-2H-pyrrolo[3,2-c]pyridazine-7-carboxamide;
Example 177: 5-(cyclopropylmethyl)-N-methyl-2-(2-methyl-2H-indazol-5-yl)-4-(6-methylpyridin-3-yl)-3-oxo-3,5-dihydro-2H-pyrrolo[3,2-c]pyridazine-7-carboxamide;
Example 178: 5-(cyclopropylmethyl)-4-(6-cyclopropylpyridin-3-yl)-N,N-dimethyl-2-(2-methyl-2H-indazol-5-yl)-3-oxo-3,5-dihydro-2H-pyrrolo[3,2-c]pyridazine-7-sulfonamide;
Example 179: 5-(cyclopropylmethyl)-4-(4-cyclopropylphenyl)-N-methyl-2-(2-methyl-2H-indazol-5-yl)-3-oxo-3,5-dihydro-2H-pyrrolo[3,2-c]pyridazine-7-sulfonamide;
Example 180: 5-(cyclopropylmethyl)-4-(4-cyclopropylphenyl)-2-(2-methyl-2H-indazol-5-yl)-3-oxo-3,5-dihydro-2H-pyrrolo[3,2-c]pyridazine-7-sulfonamide;
Example 181: 5-(cyclopropylmethyl)-4-(6-cyclopropylpyridin-3-yl)-N-methyl-2-(2-methyl-2H-indazol-5-yl)-3-oxo-3,5-dihydro-2H-pyrrolo[3,2-c]pyridazine-7-sulfonamide;
Example 182: 5-(cyclopropylmethyl)-N-methyl-2-(2-methyl-2H-indazol-5-yl)-4-(6-methylpyridin-3-yl)-3-oxo-3,5-dihydro-2H-pyrrolo[3,2-c]pyridazine-7-sulfonamide;
Example 183: 5-(cyclopropylmethyl)-4-(4-(difluoromethoxy)phenyl)-N-methyl-2-(2-methyl-2H-indazol-5-yl)-3-oxo-3,5-dihydro-2H-pyrrolo[3,2-c]pyridazine-7-sulfonamide;
Example 184: 5-(cyclopropylmethyl)-4-(4-(difluoromethoxy)phenyl)-2-(2-methyl-2H-indazol-5-yl)-3-oxo-3,5-dihydro-2H-pyrrolo[3,2-c]pyridazine-7-sulfonamide;
Example 185: 5-(cyclopropylmethyl)-4-(6-cyclopropylpyridin-3-yl)-2-(2-methyl-2H-indazol-5-yl)-3-oxo-3,5-dihydro-2H-pyrrolo[3,2-c]pyridazine-7-sulfonamide;
Example 223: 5-(cyclopropylmethyl)-4-(4-(difluoromethoxy)phenyl)-2-(1-methyl-6-oxo-1,6-dihydropyridin-3-yl)-2,5-dihydro-3H-pyrrolo[3,2-c]pyridazin-3-one;
Example 224: 2-(benzo[d]thiazol-6-yl)-5-(cyclopropylmethyl)-4-(4-(difluoromethoxy)phenyl)-2,5-dihydro-3H-pyrrolo[3,2-c]pyridazin-3-one;
Example 225: 5-(cyclopropylmethyl)-4-(4-(difluoromethoxy)phenyl)-2-(quinolin-6-yl)-2,5-dihydro-3H-pyrrolo[3,2-c]pyridazin-3-one;
Example 226: 5-(cyclopropylmethyl)-4-(4-(difluoromethoxy)phenyl)-2-(pyridin-4-yl)-2,5-dihydro-3H-pyrrolo[3,2-c]pyridazin-3-one;
Example 227: 5-(cyclopropylmethyl)-4-(4-(difluoromethoxy)phenyl)-2-(4-methyl-3,4-dihydro-2H-benzo[b][1,4]oxazin-6-yl)-2,5-dihydro-3H-pyrrolo[3,2-c]pyridazin-3-one;
Example 228: 2-(4-aminophenyl)-5-(cyclopropylmethyl)-4-(4-(difluoromethoxy)phenyl)-2,5-dihydro-3H-pyrrolo[3,2-c]pyridazin-3-one;
Example 229: 5-(cyclopropylmethyl)-4-(4-(difluoromethoxy)phenyl)-2-(4-methoxyphenyl)-2,5-dihydro-3H-pyrrolo[3,2-c]pyridazin-3-one;
Example 230: 5-(cyclopropylmethyl)-4-(4-(difluoromethoxy)phenyl)-2-(4-methoxycyclohex-1-en-1-yl)-2,5-dihydro-3H-pyrrolo[3,2-c]pyridazin-3-one;
Example 231: 5-(cyclopropylmethyl)-4-(4-(difluoromethoxy)phenyl)-2-(1H-pyrazol-4-yl)-2,5-dihydro-3H-pyrrolo[3,2-c]pyridazin-3-one;
Example 232: 5-(cyclopropylmethyl)-4-(6-cyclopropylpyridin-3-yl)-2-(1-methyl-1,2,3,6-tetrahydropyridin-4-yl)-2,5-dihydro-3H-pyrrolo[3,2-c]pyridazin-3-one;
Example 233: 7-chloro-5-(cyclopropylmethyl)-4-(6-cyclopropylpyridin-3-yl)-2-(2-methyl-2H-indazol-5-yl)-2,5-dihydro-3H-pyrrolo[3,2-c]pyridazin-3-one;
Example 234: 5-(cyclopropylmethyl)-4-(6-cyclopropylpyridin-3-yl)-7-methyl-2-(2-methyl-2H-indazol-5-yl)-2,5-dihydro-3H-pyrrolo[3,2-c]pyridazin-3-one;
Example 235: 5-(cyclopropylmethyl)-4-(6-cyclopropylpyridin-3-yl)-7-fluoro-2-(2-methyl-2H-indazol-5-yl)-2,5-dihydro-3H-pyrrolo[3,2-c]pyridazin-3-one;
Example 236: 7-acetyl-5-(cyclopropylmethyl)-4-(6-cyclopropylpyridin-3-yl)-2-(2-methyl-2H-indazol-5-yl)-2,5-dihydro-3H-pyrrolo[3,2-c]pyridazin-3-one;
Example 237: 5-(cyclopropylmethyl)-7-fluoro-2-(2-methyl-2H-indazol-5-yl)-4-(6-methylpyridin-3-yl)-2,5-dihydro-3H-pyrrolo[3,2-c]pyridazin-3-one;
Example 238: 7-bromo-5-(cyclopropylmethyl)-4-(6-cyclopropylpyridin-3-yl)-2-(2-methyl-2H-indazol-5-yl)-2,5-dihydro-3H-pyrrolo[3,2-c]pyridazin-3-one;
Example 239: 5-(cyclopropylmethyl)-4-(6-cyclopropylpyridin-3-yl)-7-(hydroxymethyl)-2-(2-methyl-2H-indazol-5-yl)-2,5-dihydro-3H-pyrrolo[3,2-c]pyridazin-3-one;
Example 240: 5-(cyclopropylmethyl)-4-(6-cyclopropylpyridin-3-yl)-7-(difluoromethyl)-2-(2-methyl-2H-indazol-5-yl)-2,5-dihydro-3H-pyrrolo[3,2-c]pyridazin-3-one;
Example 241: 5-(cyclopropylmethyl)-4-(6-cyclopropylpyridin-3-yl)-2-(2-methyl-3-(trifluoromethyl)-2H-indazol-5-yl)-2,5-dihydro-3H-pyrrolo[3,2-c]pyridazin-3-one:
Example 242: 5-(cyclopropylmethyl)-4-(6-cyclopropylpyridin-3-yl)-2-(2-methyl-3-(trifluoromethyl)-2H-indazol-5-yl)-7-(trifluoromethyl)-2,5-dihydro-3H-pyrrolo[3,2-c]pyridazin-3-one;
Example 243: 5-(cyclopropylmethyl)-4-(6-cyclopropylpyridin-3-yl)-2-(2-methyl-2H-indazol-5-yl)-7-((trimethylsilyl)ethynyl)-2,5-dihydro-3H-pyrrolo[3,2-c]pyridazin-3-one;
Example 244: 5-(cyclopropylmethyl)-4-(6-cyclopropylpyridin-3-yl)-7-ethynyl-2-(2-methyl-2H-indazol-5-yl)-2,5-dihydro-3H-pyrrolo[3,2-c]pyridazin-3-one;
Example 245: 5-(cyclopropylmethyl)-4-(6-cyclopropylpyridin-3-yl)-2-(2-methyl-2H-indazol-5-yl)-7-(3,3,3-trifluoroprop-1-yn-1-yl)-2,5-dihydro-3H-pyrrolo[3,2-c]pyridazin-3-one;
Example 246: 5-(cyclopropylmethyl)-4-(6-cyclopropylpyridin-3-yl)-2-(2-methyl-2H-indazol-5-yl)-7-(trifluoromethyl)-2,5-dihydro-3H-pyrrolo[3,2-c]pyridazin-3-one;
Example 247: 5-(cyclopropylmethyl)-4-(6-cyclopropylpyridin-3-yl)-7-ethyl-2-(2-methyl-2H-indazol-5-yl)-2,5-dihydro-3H-pyrrolo[3,2-c]pyridazin-3-one;
Example 248: 5-(cyclopropylmethyl)-4-(6-cyclopropylpyridin-3-yl)-7-isopropyl-2-(2-methyl-2H-indazol-5-yl)-2,5-dihydro-3H-pyrrolo[3,2-c]pyridazin-3-one;
Example 249: (R)-5-(cyclopropylmethyl)-4-(6-cyclopropylpyridin-3-yl)-7-(1-hydroxyethyl)-2-(2-methyl-2H-indazol-5-yl)-2,5-dihydro-3H-pyrrolo[3,2-c]pyridazin-3-one;
Example 250: (S)-5-(cyclopropylmethyl)-4-(6-cyclopropylpyridin-3-yl)-7-(1-hydroxyethyl)-2-(2-methyl-2H-indazol-5-yl)-2,5-dihydro-3H-pyrrolo[3,2-c]pyridazin-3-one;
Example 251: 6-chloro-5-(cyclopropylmethyl)-4-(6-cyclopropylpyridin-3-yl)-2-(2-methyl-2H-indazol-5-yl)-3-oxo-3,5-dihydro-2H-pyrrolo[3,2-c]pyridazine-7-carbonitrile;
Example 252: (S)-5-(cyclopropylmethyl)-4-(6-(2,2-difluorocyclopropyl)pyridin-3-yl)-2-(2-methyl-2H-indazol-5-yl)-3-oxo-3,5-dihydro-2H-pyrrolo[3,2-c]pyridazine-7-carbonitrile;
Example 253: (R)-5-(cyclopropylmethyl)-4-(6-(2,2-difluorocyclopropyl)pyridin-3-yl)-2-(2-methyl-2H-indazol-5-yl)-3-oxo-3,5-dihydro-2H-pyrrolo[3,2-c]pyridazine-7-carbonitrile;
Example 254: 5-(cyclopropylmethyl)-2-(2-methyl-2H-indazol-5-yl)-4-[6-(1-methylcyclopropyl)pyridin-3-yl]-3-oxo-2H,3H,5H-pyrrolo[3,2-c]pyridazine-7-carbonitrile;
Example 255: 5-(cyclopropylmethyl)-7-(hydroxymethyl)-2-(2-methyl-2H-indazol-5-yl)-4-(6-methylpyridin-3-yl)-2,5-dihydro-3H-pyrrolo[3,2-c]pyridazin-3-one;
Example 256: 5-(cyclopropylmethyl)-4-(6-(1-hydroxycyclopropyl)pyridin-3-yl)-2-(2-methyl-2H-indazol-5-yl)-3-oxo-3,5-dihydro-2H-pyrrolo[3,2-c]pyridazine-7-carbonitrile;
Example 257: 5-(cyclopropylmethyl)-4-(6-(3-fluoroprop-1-en-2-yl)pyridin-3-yl)-2-(2-methyl-2H-indazol-5-yl)-3-oxo-3,5-dihydro-2H-pyrrolo[3,2-c]pyridazine-7-carbonitrile;
Example 258: 5-(cyclopropylmethyl)-2-(2-methyl-2H-indazol-5-yl)-4-(6-methylpyridin-3-yl)-7-(trifluoromethyl)-2,5-dihydro-3H-pyrrolo[3,2-c]pyridazin-3-one;
Example 259: 2-(5-(cyclopropylmethyl)-4-(6-cyclopropylpyridin-3-yl)-2-(2-methyl-2H-indazol-5-yl)-3-oxo-3,5-dihydro-2H-pyrrolo[3,2-c]pyridazin-7-yl)acetonitrile;
Example 261: 5-(cyclopropylmethyl)-4-(6-cyclopropylpyridin-3-yl)-2-(2-methyl-2H-indazol-5-yl)-7-(methylsulfonyl)-2,5-dihydro-3H-pyrrolo[3,2-c]pyridazin-3-one;
Example 262: 5-(cyclopropylmethyl)-4-(4-cyclopropylphenyl)-2-(2-methyl-2H-indazol-5-yl)-7-(methylsulfonyl)-2,5-dihydro-3H-pyrrolo[3,2-c]pyridazin-3-one;
Example 263: 5-(cyclopropylmethyl)-4-(6-cyclopropylpyridin-3-yl)-6-methyl-2-(2-methyl-2H-indazol-5-yl)-2,5-dihydro-3H-pyrrolo[3,2-c]pyridazin-3-one;
Example 264: 5-(cyclopropylmethyl)-4-(4-(difluoromethoxy)phenyl)-6-methyl-2-(2-methyl-2H-indazol-5-yl)-3-oxo-3,5-dihydro-2H-pyrrolo[3,2-c]pyridazine-7-carbonitrile;
Example 265: 5-(cyclopropylmethyl)-4-(6-cyclopropylpyridin-3-yl)-6-methyl-2-(2-methyl-2H-indazol-5-yl)-3-oxo-2H,3H,5H-pyrrolopyridazine-7-carbonitrile;
Example 266: 5-(cyclopropylmethyl)-6-methyl-2-(2-methyl-2H-indazol-5-yl)-4-(6-methylpyridin-3-yl)-3-oxo-2H,3H,5H-pyrrolopyridazine-7-carbonitrile;
Example 267: 5-(cyclopropylmethyl)-4-(6-cyclopropylpyridin-3-yl)-2-(2-methyl-2H-indazol-5-yl)-7-((methylamino)methyl)-2,5-dihydro-3H-pyrrolo[3,2-c]pyridazin-3-one;
Example 268: (S)-4-(6-cyclopropylpyridin-3-yl)-5-((2,2-difluorocyclopropyl)methyl)-2-(2-methyl-2H-indazol-5-yl)-3-oxo-3,5-dihydro-2H-pyrrolo[3,2-c]pyridazine-7-carbonitrile;
Example 269: (R)-4-(6-cyclopropylpyridin-3-yl)-5-((2,2-difluorocyclopropyl)methyl)-2-(2-methyl-2H-indazol-5-yl)-3-oxo-3,5-dihydro-2H-pyrrolo[3,2-c]pyridazine-7-carbonitrile;
Example 270: 5-(cyclopropylmethyl)-4-(6-cyclopropylpyridin-3-yl)-2-(2-(methyl-d3)-2H-indazol-5-yl)-3-oxo-3,5-dihydro-2H-pyrrolo[3,2-c]pyridazine-7-carbonitrile;
Example 186: 5-(cyclopropylmethyl)-4-[4-(difluoromethoxy)phenyl]-2-(2-methyl-2H-indazol-5-yl)-2H,3H,5H-imidazo[4,5-c]pyridazin-3-one;
Example 187: 5-(cyclopropylmethyl)-2-(2-methyl-2H-indazol-5-yl)-4-(6-methylpyridin-3-yl)-2,5-dihydro-3H-imidazo[4,5-c]pyridazin-3-one;
Example 188: 4-(4-chlorophenyl)-5-(cyclopropylmethyl)-2-(2-methyl-2H-indazol-5-yl)-2,5-dihydro-3H-imidazo[4,5-c]pyridazin-3-one;
Example 189: 5-(cyclopropylmethyl)-4-(6-(difluoromethoxy)pyridin-3-yl)-2-(2-methyl-2H-indazol-5-yl)-2,5-dihydro-3H-imidazo[4,5-c]pyridazin-3-one;
Example 190: 5-(cyclopropylmethyl)-4-(6-cyclopropylpyridin-3-yl)-2-(2-methyl-2H-indazol-5-yl)-2,5-dihydro-3H-imidazo[4,5-c]pyridazin-3-one;
Example 191: 5-(cyclopropylmethyl)-4-(4-(difluoromethoxy)phenyl)-6-methyl-2-(2-methyl-2H-indazol-5-yl)-2,5-dihydro-3H-imidazo[4,5-c]pyridazin-3-one;
Example 192: 5-(cyclopropylmethyl)-4-(4-cyclopropylphenyl)-2-(2-methyl-2H-indazol-5-yl)-2,5-dihydro-3H-imidazo[4,5-c]pyridazin-3-one;
Example 193: 5-(cyclopropylmethyl)-4-(4-methoxyphenyl)-2-(2-methyl-2H-indazol-5-yl)-2,5-dihydro-3H-imidazo[4,5-c]pyridazin-3-one;
Example 194: 5-(cyclopropylmethyl)-4-(4-(methoxy-d3)phenyl)-2-(2-methyl-2H-indazol-5-yl)-2,5-dihydro-3H-imidazo[4,5-c]pyridazin-3-one;
Example 195: 5-(cyclopropylmethyl)-2-(1,2-dimethyl-1H-benzo[d]imidazol-6-yl)-4-(4-methoxyphenyl)-2,5-dihydro-3H-imidazo[4,5-c]pyridazin-3-one;
Example 196: 5-(cyclopropylmethyl)-4-[4-(difluoromethoxy)phenyl]-7-methyl-2-(2-methyl-2H-indazol-5-yl)-2H,3H,5H,6H,7H-imidazolidino[4,5-c]pyridazine-3,6-dione;
Example 197: 5-(cyclopropylmethyl)-4-(4-(difluoromethoxy)phenyl)-2-(2-methyl-2H-indazol-5-yl)-2,7-dihydro-3H-imidazo[4,5-c]pyridazine-3,6(5H)-dione;
Example 198: 5-(cyclopropylmethyl)-4-(6-cyclopropylpyridin-3-yl)-7-methyl-2-(2-methyl-2H-indazol-5-yl)-2,7-dihydro-3H-imidazo[4,5-c]pyridazine-3,6(5H)-dione;
Example 199: 4-(4-chlorophenyl)-5-(cyclopropylmethyl)-7-methyl-2-(2-methyl-2H-indazol-5-yl)-2,7-dihydro-3H-imidazo[4,5-c]pyridazine-3,6(5H)-dione;
Example 200: 5-(cyclopropylmethyl)-4-(4-cyclopropylphenyl)-7-methyl-2-(2-methyl-2H-indazol-5-yl)-2,7-dihydro-3H-imidazo[4,5-c]pyridazine-3,6(5H)-dione;
Example 201: 5-(cyclopropylmethyl)-4-(4-(methoxy-d3)phenyl)-7-methyl-2-(2-methyl-2H-indazol-5-yl)-2,7-dihydro-3H-imidazo[4,5-c]pyridazine-3,6(5H)-dione;
Example 202: 5-(cyclopropylmethyl)-7-methyl-2-(2-methyl-2H-indazol-5-yl)-4-(6-methylpyridin-3-yl)-2,7-dihydro-3H-imidazo[4,5-c]pyridazine-3,6(5H)-dione;
Example 203: 5-(cyclopropylmethyl)-4-(6-cyclopropylpyridin-3-yl)-2-(2-methyl-2H-indazol-5-yl)-2,7-dihydro-3H-imidazo[4,5-c]pyridazine-3,6(5H)-dione;
Example 204: 5-(cyclopropylmethyl)-4-(4-cyclopropylphenyl)-2-(2-methyl-2H-indazol-5-yl)-2,7-dihydro-3H-imidazo[4,5-c]pyridazine-3,6(5H)-dione;
Example 205: 4-(4-chlorophenyl)-5-(cyclopropylmethyl)-2-(2-methyl-2H-indazol-5-yl)-2,7-dihydro-3H-imidazo[4,5-c]pyridazine-3,6(5H)-dione;
Example 206: 5-(cyclopropylmethyl)-4-(4-(methoxy-d3)phenyl)-2-(2-methyl-2H-indazol-5-yl)-2,7-dihydro-3H-imidazo[4,5-c]pyridazine-3,6(5H)-dione;
Example 207: 5-(cyclopropylmethyl)-2-(2-methyl-2H-indazol-5-yl)-4-(6-methylpyridin-3-yl)-2,7-dihydro-3H-imidazo[4,5-c]pyridazine-3,6(5H)-dione;
Example 208: 1-(cyclopropylmethyl)-7-(4-(difluoromethoxy)phenyl)-5-(2-methyl-2H-indazol-5-yl)-1,5-dihydro-6H-[1,2,3]triazolo[4,5-c]pyridazin-6-one;
Example 209: 1-(cyclopropylmethyl)-7-(4-cyclopropylphenyl)-5-(2-methyl-2H-indazol-5-yl)-1,5-dihydro-6H-[1,2,3]triazolo[4,5-c]pyridazin-6-one;
Example 210 1-(cyclopropylmethyl)-7-(6-cyclopropylpyridin-3-yl)-5-(2-methyl-2H-indazol-5-yl)-1,5-dihydro-6H-[1,2,3]triazolo[4,5-c]pyridazin-6-one;
Example 211: 1-(cyclopropylmethyl)-7-(6-cyclopropylpyridin-3-yl)-3-methyl-5-(2-methyl-2H-indazol-5-yl)-1H,5H,6H-pyrazolo[4,3-c]pyridazin-6-one;
Example 212: 1-(cyclopropylmethyl)-7-(4-(difluoromethoxy)phenyl)-3-methyl-5-(2-methyl-2H-indazol-5-yl)-1,5-dihydro-6H-pyrazolo[4,3-c]pyridazin-6-one;
Example 213: 1-(cyclopropylmethyl)-7-(4-cyclopropylphenyl)-3-methyl-5-(2-methyl-2H-indazol-5-yl)-1,5-dihydro-6H-pyrazolo[4,3-c]pyridazin-6-one;
Example 214: 1-(cyclopropylmethyl)-7-(4-methoxyphenyl)-3-methyl-5-(2-methyl-2H-indazol-5-yl)-1,5-dihydro-6H-pyrazolo[4,3-c]pyridazin-6-one;
Example 215: 7-(4-chlorophenyl)-1-(cyclopropylmethyl)-3-methyl-5-(2-methyl-2H-indazol-5-yl)-1,5-dihydro-6H-pyrazolo[4,3-c]pyridazin-6-one;
Example 216: 1-(cyclopropylmethyl)-7-(4-(methoxy-d3)phenyl)-3-methyl-5-(2-methyl-2H-indazol-5-yl)-1,5-dihydro-6H-pyrazolo[4,3-c]pyridazin-6-one;
Example 217: 1-(cyclopropylmethyl)-3-methyl-5-(2-methyl-2H-indazol-5-yl)-7-(6-methylpyridin-3-yl)-1,5-dihydro-6H-pyrazolo[4,3-c]pyridazin-6-one;
Example 218: 1-(cyclopropylmethyl)-7-(4-(difluoromethoxy)phenyl)-5-(2-methyl-2H-indazol-5-yl)-1,5-dihydro-6H-pyrazolo[4,3-c]pyridazin-6-one;
Example 219: 1-(cyclopropylmethyl)-7-(6-cyclopropylpyridin-3-yl)-5-(2-methyl-2H-indazol-5-yl)-1,5-dihydro-6H-pyrazolo[4,3-c]pyridazin-6-one;
Example 220: 1-(cyclopropylmethyl)-7-(4-(methoxy-d3)phenyl)-5-(2-methyl-2H-indazol-5-yl)-1,5-dihydro-6H-pyrazolo[4,3-c]pyridazin-6-one;
Example 221: 7-(4-chlorophenyl)-1-(cyclopropylmethyl)-5-(2-methyl-2H-indazol-5-yl)-1,5-dihydro-6H-pyrazolo[4,3-c]pyridazin-6-one;
Example 222: 1-(cyclopropylmethyl)-5-(2-methyl-2H-indazol-5-yl)-7-(6-methylpyridin-3-yl)-1,5-dihydro-6H-pyrazolo[4,3-c]pyridazin-6-one;
Example 260: 5-(cyclopropylmethyl)-4-(6-cyclopropylpyridin-3-yl)-7,7-dimethyl-2-(2-methyl-2H-indazol-5-yl)-2,5,6,7-tetrahydro-3H-pyrrolo[3,2-c]pyridazin-3-one;
Example 271: 5-(cyclopropylmethyl)-4-(4-(difluoromethoxy)phenyl)-2-(2-methyl-2H-indazol-5-yl)-2,7-dihydro-3H-pyrrolo[2,3-c]pyridazin-3-one; or
Example 272: 5-(cyclopropylmethyl)-4-(4-(difluoromethoxy)phenyl)-7-methyl-2-(2-methyl-2H-indazol-5-yl)-2,7-dihydro-3H-pyrrolo[2,3-c]pyridazin-3-one.

24. The compound of claim 1, or a pharmaceutically acceptable salt thereof, that is:

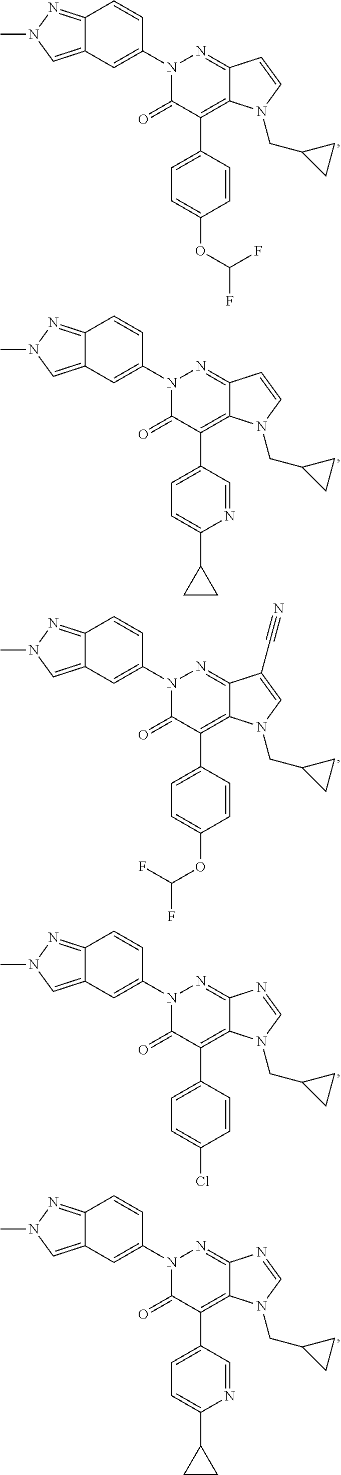

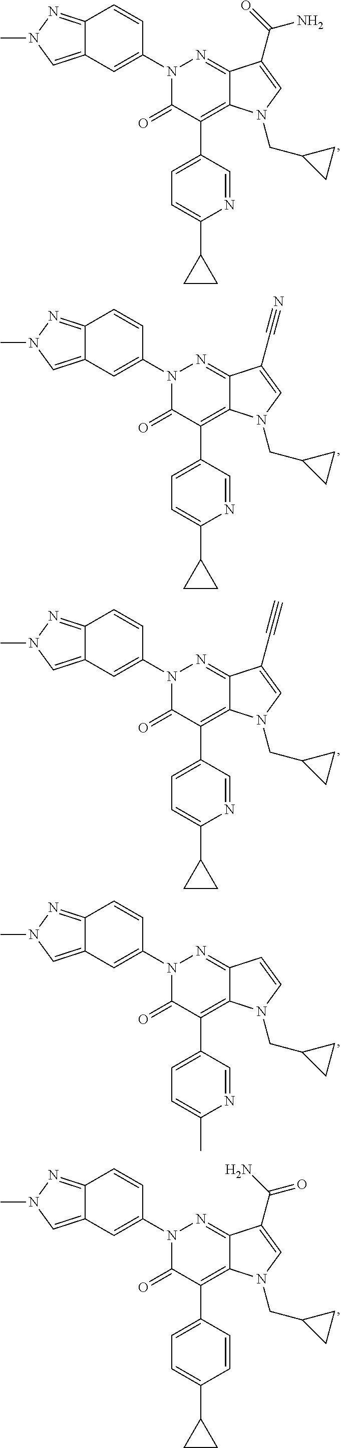 [SMILES: Cn1cc2cc(-n3nc4c(C(N)=O)cn(CC5CC5)c4c(-c4ccc(C5CC5)nc4)c3=O)ccc2n1 ; Cn1cc2cc(-n3nc4c(C#N)cn(CC5CC5)c4c(-c4ccc(C5CC5)nc4)c3=O)ccc2n1 ; C#Cc1cn(CC2CC2)c2c(-c3ccc(C4CC4)nc3)c(=O)n(-c3ccc4nn(C)cc4c3)nc12 ; Cc1ccc(-c2c(=O)n(-c3ccc4nn(C)cc4c3)nc3ccn(CC4CC4)c23)cn1 ; Cn1cc2cc(-n3nc4c(C(N)=O)cn(CC5CC5)c4c(-c4ccc(C5CC5)cc4)c3=O)ccc2n1]

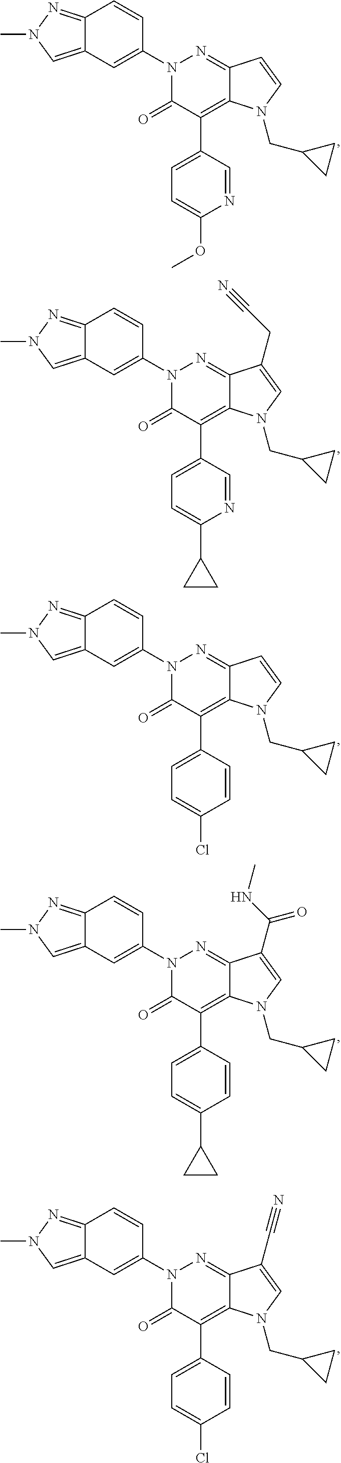

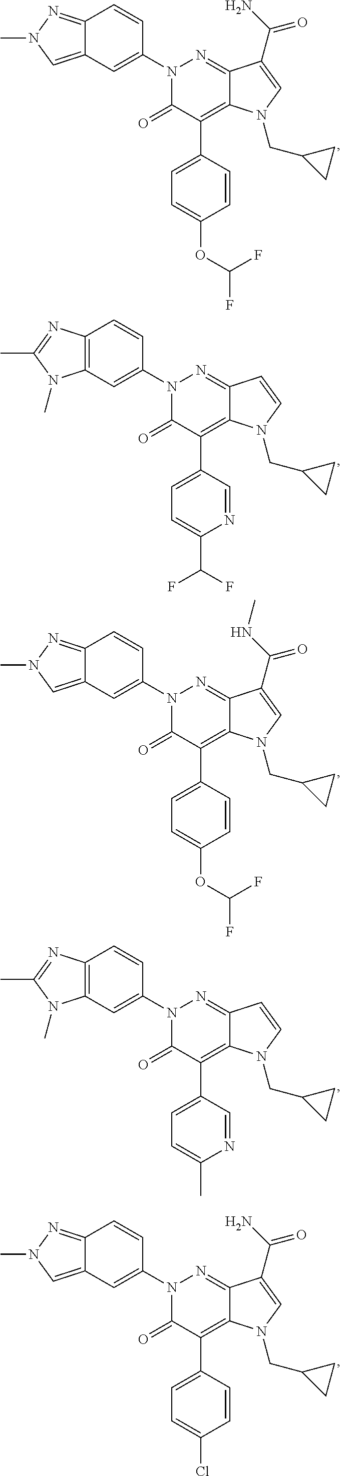

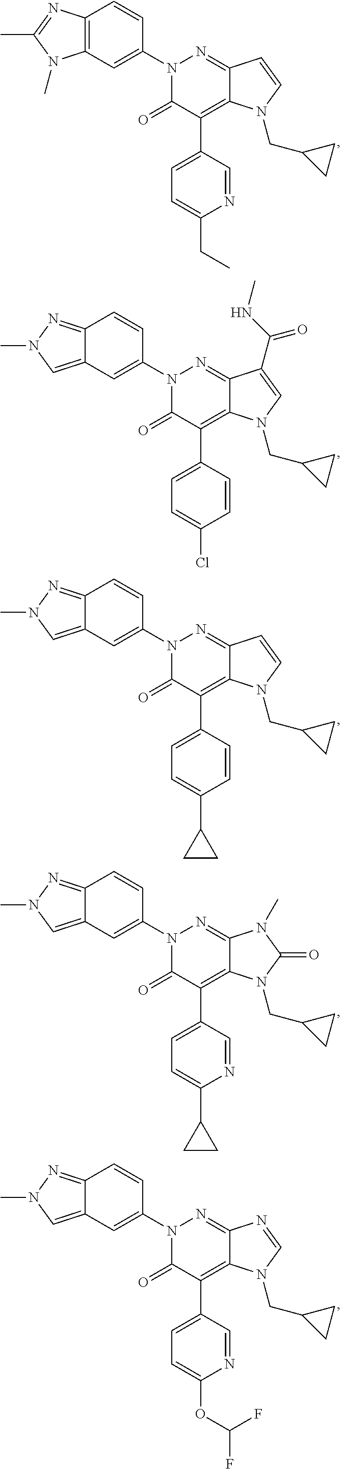

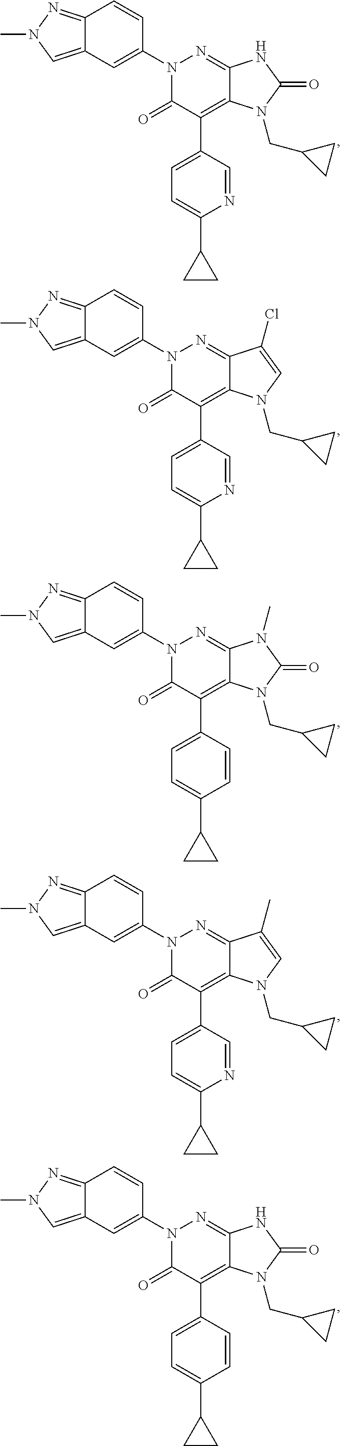 [SMILES: Cn1cc2cc(-n3nc4[nH]c(=O)n(CC5CC5)c4c(-c4ccc(C5CC5)nc4)c3=O)ccc2n1 ; Cn1cc2cc(-n3nc4c(Cl)cn(CC5CC5)c4c(-c4ccc(C5CC5)nc4)c3=O)ccc2n1 ; Cn1cc2cc(-n3nc4c(c(-c5ccc(C6CC6)cc5)c3=O)n(CC3CC3)c(=O)n4C)ccc2n1 ; Cc1cn(CC2CC2)c2c(-c3ccc(C4CC4)nc3)c(=O)n(-c3ccc4nn(C)cc4c3)nc12 ; Cn1cc2cc(-n3nc4[nH]c(=O)n(CC5CC5)c4c(-c4ccc(C5CC5)cc4)c3=O)ccc2n1]

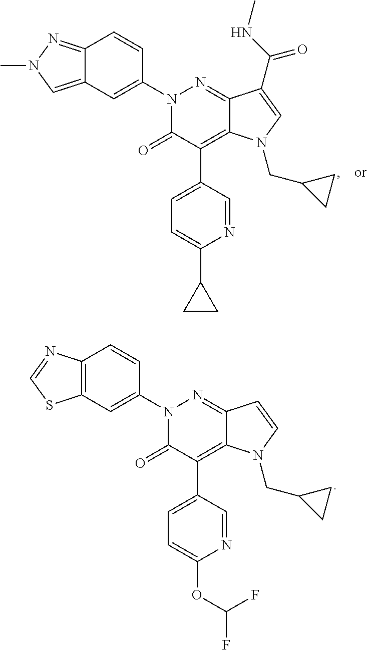 [SMILES: CNC(=O)c1cn(CC2CC2)c2c(-c3ccc(C4CC4)nc3)c(=O)n(-c3ccc4nn(C)cc4c3)nc12 ; O=c1c(-c2ccc(OC(F)F)nc2)c2c(ccn2CC2CC2)nn1-c1ccc2ncsc2c1]

25. A method for inhibiting the synthesis of S-adenosyl methionine (SAM) or treating a disease or condition that responds to the inhibition of MAT2A, or in a subject in need thereof, comprising administering to the subject a SAM-synthesis inhibiting amount of a compound, or a pharmaceutically acceptable salt thereof, of claim 1.

26. The method of claim 25, wherein said disease or condition is cancer.

27. The method of claim 26, wherein the cancer is selected from the group consisting of neuroblastoma, rectum carcinoma, colon carcinoma, familiary adenomatous polyposis carcinoma and hereditary non-polyposis colorectal cancer, esophageal carcinoma, labial carcinoma, larynx carcinoma, hypopharynx carcinoma, tong carcinoma, salivary gland carcinoma, gastric carcinoma, adenocarcinoma, medullary thyroidea carcinoma, papillary thyroidea carcinoma, renal carcinoma, kidney parenchym carcinoma, ovarian carcinoma, cervix carcinoma, uterine corpus carcinoma, endometrium carcinoma, chorion carcinoma, pancreatic carcinoma, prostate carcinoma, testis carcinoma, breast carcinoma, urinary carcinoma, melanoma, brain tumors such as glioblastoma, astrocytoma, meningioma, medulloblastoma and peripheral neuroectodermal tumors, Hodgkin lymphoma, non-Hodgkin lymphoma, Burkitt lymphoma, acute lymphatic leukemia (ALL), chronic lymphatic leukemia (CLL), acute myeloid leukemia (AML), chronic myeloid leukemia (CML), adult T-cell leukemia, lymphoma, hepatocellular carcinoma, gall bladder carcinoma, bronchial carcinoma, small cell lung carcinoma, non-small cell lung carcinoma, multiple myeloma, basalioma, teratoma, retinoblastoma, choroidea melanoma, seminoma, rhabdomyo sarcoma, craniopharyngioma, osteosarcoma, chondrosarcoma, myosarcoma, liposarcoma, fibrosarcoma, Ewing sarcoma, and plasmocytoma.

28. The method of claim 26, wherein the cancer is characterized by a reduction or absence of methylthioadenosine phosphorylase (MTAP) gene expression, the absence of the MTAP gene, or reduced function of MTAP protein.

29. The method of claim 26, wherein the cancer is MTAP null, has a KRAS mutation, or has a p53 mutation.

30. The method of claim 29, wherein the KRAS mutation is an amino acid substitution at residue 12 or 13 or the p53 mutation is Y126_splice, K132Q, M133K, R174fs, R175H, R196*, C238S, C242Y, G245S, R248W, R248Q, 1255T, D259V, S261_splice, R267P, R273C, R282W, A159V or R280K.

31. The method of claim 29, wherein said KRAS mutation is G12C, G12R, G12V or G13D.

32. A pharmaceutical composition comprising a compound, or a pharmaceutically acceptable salt thereof, of claim 1, and a pharmaceutically acceptable carrier.
